SEQUENCE LISTING (727 sequences)

SEQ ID NO: 1 - protein, 179 aa, synthetic construct
SRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGS

SEQ ID NO: 2 - DNA, 537 bp, synthetic construct
  source 1..537: mol_type = other DNA
tcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtct

SEQ ID NO: 3 - protein, 178 aa, synthetic construct
ERPFQCRICMRNFSHKQHRDAHIRTHTGEKPFACDICGRKFARSANLTRHTKIHTGSQKPFQCRICMRNFSRSDNLSEHIRTHTGEKPFACDICGRKFATSANLSRHTKIHTGSQKPFQCRICMRNFSIRSTLRDHIRTHTGEKPFACDICGRKFARTPVRMGHTKIHLRQKDAARGS

SEQ ID NO: 4 - DNA, 534 bp, synthetic construct
  source 1..534: mol_type = other DNA
gagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatct

SEQ ID NO: 5 - DNA, 19 bp, synthetic construct
  source 1..19: mol_type = other DNA
gcaggaggacgaggacggc

SEQ ID NO: 6 - DNA, 20 bp, synthetic construct
  source 1..20: mol_type = other DNA
gctgcctgatagggagcgtc

SEQ ID NO: 7 - RNA, 112 nt, synthetic construct
  source 1..112: mol_type = other RNA
  modified_base order(1..4,109..112): mod_base = OTHER; note = phosphorothioate linkage
gcaacaccgcctagaccgacgtttaagagctaagctggaaacagcatagcaagtttaaataaggctagtccgttatcaacttgaaaaagtggcaccgagtcggtgctttttt

SEQ ID NO: 8 - RNA, 112 nt, synthetic construct
  source 1..112: mol_type = other RNA
  modified_base order(1..4,109..112): mod_base = OTHER; note = phosphorothioate linkage
actgcctggctcactcctccgtttaagagctaagctggaaacagcatagcaagtttaaataaggctagtccgttatcaacttgaaaaagtggcaccgagtcggtgctttttt

SEQ ID NO: 9 - protein, 2275 aa, synthetic construct
MYPYDVPDYASPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESRTLVTFKDVFVDFTREEWKLLDTAQQIVYRNVMLENYKNLVSLGYQLTKPDVILRLEKGEEPWLV

SEQ ID NO: 10 - DNA, 6825 bp, synthetic construct
  source 1..6825: mol_type = other DNA
atgtacccatacgatgttccagattacgcttcgccgaagaaaaagcgcaaggtcaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtcccggaccctggtgacattcaaggacgtgttcgtggacttcacccgggaggagtggaagctgctggacacagcccagcagatcgtgtacaggaacgtgatgctggagaactataagaatctggtgtctctgggctaccagctgacaaagccagatgtgatcctgcggctggagaagggagaggagccctggctggtg

SEQ ID NO: 11 - protein, 1488 aa, synthetic construct
MPKKKRKVPKKKRKVYDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGSETPGTSESATPESTGRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 12 - DNA, 4464 bp, synthetic construct
  source 1..4464: mol_type = other DNA
atgccaaaaaagaagagaaaggtaccgaagaaaaaaagaaaggtatacgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcggaagtgagaccccaggtacatccgaatcagcaacgcctgaaagcaccggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcgctgattacaaagatgatgacgataaagccccaaaaaagaagagaaaggtaccgaagaaaaaaagaaaggtc

SEQ ID NO: 13 - protein, 1118 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSSGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 14 - DNA, 3354 bp, synthetic construct
  source 1..3354: mol_type = other DNA
atgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagcggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtg

SEQ ID NO: 15 - protein, 1117 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSEERPFQCRICMRNFSHKQHRDAHIRTHTGEKPFACDICGRKFARSANLTRHTKIHTGSQKPFQCRICMRNFSRSDNLSEHIRTHTGEKPFACDICGRKFATSANLSRHTKIHTGSQKPFQCRICMRNFSIRSTLRDHIRTHTGEKPFACDICGRKFARTPVRMGHTKIHLRQKDAARGSSGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 16 - DNA, 3355 bp, synthetic construct
  source 1..3355: mol_type = other DNA
atgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgaggagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctagcggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtgaa

SEQ ID NO: 17 - protein, 1324 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSGVDGSSGSETPGTSESATPESERPFQCRICMRNFSHKQHRDAHIRTHTGEKPFACDICGRKFARSANLTRHTKIHTGSQKPFQCRICMRNFSRSDNLSEHIRTHTGEKPFACDICGRKFATSANLSRHTKIHTGSQKPFQCRICMRNFSIRSTLRDHIRTHTGEKPFACDICGRKFARTPVRMGHTKIHLRQKDAARGSSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 18 - DNA, 3972 bp, synthetic construct
  source 1..3972: mol_type = other DNA
atgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctggtgttgatggctcctctggttctgaaactcctggtacttctgagtctgctactcccgaatctgagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtg

SEQ ID NO: 19 - protein, 1324 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSEERPFQCRICMRNFSHKQHRDAHIRTHTGEKPFACDICGRKFARSANLTRHTKIHTGSQKPFQCRICMRNFSRSDNLSEHIRTHTGEKPFACDICGRKFATSANLSRHTKIHTGSQKPFQCRICMRNFSIRSTLRDHIRTHTGEKPFACDICGRKFARTPVRMGHTKIHLRQKDAARGSGVDGSSGSETPGTSESATPESSRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 20 - DNA, 3975 bp, synthetic construct
  source 1..3975: mol_type = other DNA
atgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgaggagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctggtgttgatggctcctctggttctgaaactcctggtacttctgagtctgctactcccgaatcttcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtga

SEQ ID NO: 21 - protein, 1485 aa, synthetic construct
MPKKKRKVPKKKRKVYDSVAFEDVAVNFTLEEWALLDPSQKNLYRDVMRETFRNLASVGKQWEDQNIEDPFKIPRRNISHIPERLCESKEGGQGEESGVDGSSGSETPGTSESATPESERPFQCRICMRNFSHKQHRDAHIRTHTGEKPFACDICGRKFARSANLTRHTKIHTGSQKPFQCRICMRNFSRSDNLSEHIRTHTGEKPFACDICGRKFATSANLSRHTKIHTGSQKPFQCRICMRNFSIRSTLRDHIRTHTGEKPFACDICGRKFARTPVRMGHTKIHLRQKDAARGSGGPSSGAPPPSGGSPAGSPTSTEEGTSESATPESGPGTSTEPSEGSAPGSPAGSPTSTEEGTSTEPSEGSAPGTSTEPSENHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSEPKKKRKVSRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSSGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 22 - DNA, 4455 bp, synthetic construct
  source 1..4455: mol_type = other DNA
atgccaaaaaagaagagaaaggtaccgaagaaaaaaagaaaggtatacgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcggagtcgacggatccagcggctccgagaccccaggcacatctgagagcgccacccctgagtccgagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagcccaagaagaagaggaaggtgtcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagcggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtg

SEQ ID NO: 23 - protein, 2272 aa, synthetic construct
MPKKKRKVPKKKRKVDSVAFEDVAVNFTLEEWALLDPSQKNLYRDVMRETFRNLASVGKQWEDQNIEDPFKIPRRNISHIPERLCESKEGGQGEESASGSETPGTSESATPESTGPKKKRKVSRPGERPFQCRICMRNFSXXXXXXXHXXTHTGEKPFQCRICMRNFSXXXXXXXHXXTHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFQCRICMRNFSXXXXXXXHXXTHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFQCRICMRNFSXXXXXXXHXXTHLRGSGVDGSSGSETPGTSESATPESERPFQCRICMRNFSXXXXXXXHXXTHTGEKPFACDICGRKFAXXXXXXXHXXIHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFACDICGRKFAXXXXXXXHXXIHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFACDICGRKFAXXXXXXXHXXIHLRQKDAARGSSGVDGSGGPSSGAPPPSGGSPAGSPTSTEEGTSESATPESGPGTSTEPSEGSAPGSPAGSPTSTEEGTSTEPSEGSAPGTSTEPSEYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLPKKKRKVPKKKRKV

SEQ ID NO: 24 - DNA, 5287 bp, synthetic construct
  source 1..5287: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataagggggcgctcgagcaggttcagaaggagatcaaaaacccccaaggatcaaacatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctggtgttgatggctcctctggttctgaaactcctggtacttctgagtctgctactcccgaatctgagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtgaggatcctgagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaatt

SEQ ID NO: 25 - DNA, 5287 bp, synthetic construct
  source 1..5287: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataagggggcgctcgagcaggttcagaaggagatcaaaaacccccaaggatcaaacatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgaggagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctggtgttgatggctcctctggttctgaaactcctggtacttctgagtctgctactcccgaatcttcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtgaggatcctgagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaatt

SEQ ID NO: 26 - DNA, 648 bp, synthetic construct
  source 1..648: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataagg

SEQ ID NO: 27 - DNA, 50 bp, synthetic construct
  source 1..50: mol_type = other DNA
gggcgctcgagcaggttcagaaggagatcaaaaacccccaaggatcaaac

SEQ ID NO: 28 - protein, 302 aa, synthetic construct
YNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 29 - protein, 386 aa, synthetic construct
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 30 - protein, 21 aa, synthetic construct
GVDGSSGSETPGTSESATPES

SEQ ID NO: 31 - protein, 8 aa, synthetic construct
DYKDDDDK

SEQ ID NO: 32 - DNA, 21 bp, synthetic construct
  source 1..21: mol_type = other DNA
ccgaagaaaaaaagaaaggta

SEQ ID NO: 33 - protein, 100 aa, synthetic construct
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 34 - protein, 106 aa, synthetic construct
MAATLLMAGSQAPVTFEDMAMYLTREEWRPLDAAQRDLYRDVMQENYGNVVSLDFEIRSENEVNPKQEISEDVQFGTTSERPAENAEENPESEEGFESGDRSERQW

SEQ ID NO: 35 - protein, 95 aa, synthetic construct
MLENYRNLVFLGIAVSKPDLITCLEQGKEPCNMKRHEMVAKPPVMCSHIAEDLCPERDIKYFFQKVILRRYDKCEHENLQLRKGCKSVDECKVCK

SEQ ID NO: 36 - protein, 95 aa, synthetic construct
MGLLTFRDVAIEFSLEEWQCLDTAQRNLYKNVILENYRNLVFLGIAVSKQDLITCLEQEKEPLTVKRHEMVNEPPVMCSHFAQEFWPEQNIKDSF

SEQ ID NO: 37 - protein, 116 aa, synthetic construct
MLQMQNSEHHGQSIKTQTDSISLEDVAVNFTLEEWALLDPGQRNIYRDVMRATFKNLACIGEKWKDQDIEDEHKNQGRNLRSPMVEALCENKEDCPCGKSTSQIPDLNTNLETPTG

SEQ ID NO: 38 - protein, 100 aa, synthetic construct
MALSQGLLTFRDVAIEFSQEEWKCLDPAQRTLYRDVMLENYRNLVSLDISSKCMMNTLSSTGQGNTEVIHTGTLQRQASYHIGAFCSQEIEKDIHDFVFQ

SEQ ID NO: 39 - protein, 98 aa, synthetic construct
MAQGLVTFADVAIDFSQEEWACLNSAQRDLYWDVMLENYSNLVSLDLESAYENKSLPTKKNIHEIRASKRNSDRRSKSLGRNWICEGTLERPQRSRGR

SEQ ID NO: 40 - protein, 118 aa, synthetic construct
MLREEATKKSKEKEPGMALPQGRLTFRDVAIEFSLEEWKCLNPAQRALYRAVMLENYRNLEFVDSSLKSMMEFSSTRHSITGEVIHTGTLQRHKSHHIGDFCFPEMKKDIHHFEFQWQ

SEQ ID NO: 41 - protein, 104 aa, synthetic construct
MPGPPGSLEMGPLTFRDVAIEFSLEEWQCLDTAQRNLYRKVMFENYRNLVFLGIAVSKPHLITCLEQGKEPWNRKRQEMVAKPPVIYSHFTEDLWPEHSIKDSF

SEQ ID NO: 42 - protein, 113 aa, synthetic construct
MSPPWSPALAAEGRGSSCEASVSFEDVTVDFSKEEWQHLDPAQRRLYWDVTLENYSHLLSVGYQIPKSEAAFKLEQGEGPWMLEGEAPHQSCSGEAIGKMQQQGIPGGIFFHC

SEQ ID NO: 43 - protein, 109 aa, synthetic construct
MASPSPPPESKEEWDYLDPAQRSLYKDVMMENYGNLVSLDVLNRDKDEEPTVKQEIEEIEEEVEPQGVIVTRIKSEIDQDPMGRETFELVGRLDKQRGIFLWEIPRESL

SEQ ID NO: 44 - protein, 99 aa, synthetic construct
MALTQGPLKFMDVAIEFSQEEWKCLDPAQRTLYRDVMLENYRNLVSLGICLPDLSVTSMLEQKRDPWTLQSEEKIANDPDGRECIKGVNTERSSKLGSN

SEQ ID NO: 45 - protein, 100 aa, synthetic construct
MAASAQVSVTFEDVAVTFTQEEWGQLDAAQRTLYQEVMLETCGLLMSLGCPLFKPELIYQLDHRQELWMATKDLSQSSYPGDNTKPKTTEPTFSHLALPE

SEQ ID NO: 46 - protein, 144 aa, synthetic construct
MFSQEERMAAGYLPRWSQELVTFEDVSMDFSQEEWELLEPAQKNLYREVMLENYRNVVSLEALKNQCTDVGIKEGPLSPAQTSQVTSLSSWTGYLLFQPVASSHLEQREALWIEEKGTPQASCSDWMTVLRNQDSTYKKVALQE

SEQ ID NO: 47 - protein, 97 aa, synthetic construct
MSQGSVTFRDVAIDFSQEEWKWLQPAQRDLYRCVMLENYGHLVSLGLSISKPDVVSLLEQGKEPWLGKREVKRDLFSVSESSGEIKDFSPKNVIYDD

SEQ ID NO: 48 - protein, 112 aa, synthetic construct
MEEAQKRKAKESGMALPQGRLTFMDVAIEFSQEEWKSLDPGQRALYRDVMLENYRNLVFLGRSCVLGSNAENKPIKNQLGLTLESHLSELQLFQAGRKIYRSNQVEKFTNHR

SEQ ID NO: 49 - protein, 105 aa, synthetic construct
MAAAVLTDRAQVSVTFDDVAVTFTKEEWGQLDLAQRTLYQEVMLENCGLLVSLGCPVPKAELICHLEHGQEPWTRKEDLSQDTCPGDKGKPKTTEPTTCEPALSE

SEQ ID NO: 50 - protein, 99 aa, synthetic construct
MIQAQESITLEDVAVDFTWEEWQLLGAAQKDLYRDVMLENYSNLVAVGYQASKPDALFKLEQGEQLWTIEDGIHSGACSDIWKVDHVLERLQSESLVNR

SEQ ID NO: 51 - protein, 112 aa, synthetic construct
MEGVAGVMSVGPPAARLQEPVTFRDVAVDFTQEEWGQLDPTQRILYRDVMLETFGHLLSIGPELPKPEVISQLEQGTELWVAERGTTQGCHPAWEPRSESQASRKEEGLPEE

SEQ ID NO: 52 - protein, 97 aa, synthetic construct
MSLGSELFRDVAIVFSQEEWQWLAPAQRDLYRDVMLETYSNLVSLGLAVSKPDVISFLEQGKEPWMVERVVSGGLCPVLESRYDTKELFPKQHVYEV

SEQ ID NO: 53 - protein, 105 aa, synthetic construct
MAHKYVGLQYHGSVTFEDVAIAFSQQEWESLDSSQRGLYRDVMLENYRNLVSMAGHSRSKPHVIALLEQWKEPEVTVRKDGRRWCTDLQLEDDTIGCKEMPTSEN

SEQ ID NO: 54 - protein, 92 aa, synthetic construct
MAFEDVAVYFSQEEWGLLDTAQRALYRRVMLDNFALVASLGLSTSRPRVVIQLERGEEPWVPSGTDTTLSRTTYRRRNPGSWSLTEDRDVSG

SEQ ID NO: 55 - protein, 104 aa, synthetic construct
MLENYRNLVFVGIAASKPDLITCLEQGKEPWNVKRHEMVTEPPVVYSYFAQDLWPKQGKKNYFQKVILRTYKKCGRENLQLRKYCKSMDECKVHKECYNGLNQC

SEQ ID NO: 56 - protein, 127 aa, synthetic construct
MHFRRPDPCREPLASPIQDSVAFEDVAVNFTQEEWALLDSSQKNLYREVMQETCRNLASVGSQWKDQNIEDHFEKPGKDIRNHIVQRLCESKEDGQYGEVVSQIPNLDLNENISTGLKPCECSICGK

SEQ ID NO: 57 - protein, 110 aa, synthetic construct
MALSQGLFTFKDVAIEFSQEEWECLDPAQRALYRDVMLENYRNLLSLDEDNIPPEDDISVGFTSKGLSPKENNKEELYHLVILERKESHGINNFDLKEVWENMPKFDSLW

SEQ ID NO: 58 - protein, 98 aa, synthetic construct
MTFEDIIVDFTQEEWALLDTSQRKLFQDVMLENISHLVSIGKQLCKSVVLSQLEQVEKLSTQRISLLQGREVGIKHQEIPFIHHIYQKGTSTISTMRS

SEQ ID NO: 59 - protein, 103 aa, synthetic construct
MAVTFEDVTIIFTWEEWKFLDSSQKRLYREVMWENYTNVMSVENWNESYKSQEEKFRYLEYENFSYWQGWWNAGAQMYENQNYGETVQGTDSKDLTQQDRSQC

SEQ ID NO: 60 - protein, 99 aa, synthetic construct
MITSQGSVSFRDVTVGFTQEEWQHLDPAQRTLYRDVMLENYSHLVSVGYCIPKPEVILKLEKGEEPWILEEKFPSQSHLELINTSRNYSIMKFNEFNKG

SEQ ID NO: 61 - protein, 114 aa, synthetic construct
MFEDVAVYLSREEWGRLGPAQRGLYRDVMLETYGNLVSLGVGPAGPKPGVISQLERGDEPWVLDVQGTSGKEHLRVNSPALGTRTEYKELTSQETFGEEDPQGSEPVEACDHIS

SEQ ID NO: 62 - protein, 112 aa, synthetic construct
METYGNVVSLGLPGSKPDIISQLERGEDPWVLDRKGAKKSQGLWSDYSDNLKYDHTTACTQQDSLSCPWECETKGESQNTDLSPKPLISEQTVILGKTPLGRIDQENNETKQ

SEQ ID NO: 63 - protein, 102 aa, synthetic construct
MAEMNPAQGHVVFEDVAIYFSQEEWGHLDEAQRLLYRDVMLENLALLSSLGCCHGAEDEEAPLEPGVSVGVSQVMAPKPCLSTQNTQPCETCSSLLKDILRL

SEQ ID NO: 64 - protein, 62 aa, synthetic construct
MLDNYRNLVFLGIAVSKPDLITCLEQGKEPCNMKRHAMVAKPPVVCSHFAQDLWPKQGLKDS

SEQ ID NO: 65 - protein, 105 aa, synthetic construct
MAAGQREARPQVSLTFEDVAVLFTRDEWRKLAPSQRNLYRDVMLENYRNLVSLGLPFTKPKVISLLQQGEDPWEVEKDGSGVSSLGSKSSHKTTKSTQTQDSSFQ

SEQ ID NO: 66 - protein, 97 aa, synthetic construct
MALRSVMFSDVSIDFSPEEWEYLDLEQKDLYRDVMLENYSNLVSLGCFISKPDVISSLEQGKEPWKVVRKGRRQYPDLETKYETKKLSLENDIYEIN

SEQ ID NO: 67 - protein, 98 aa, synthetic construct
MTTFKEAMTFKDVAVVFTEEELGLLDLAQRKLYRDVMLENFRNLLSVGHQAFHRDTFHFLREEKIWMMKTAIQREGNSGDKIQTEMETVSEAGTHQEW

SEQ ID NO: 68 - protein, 103 aa, synthetic construct
MFQVEQKSQESVSFKDVTVGFTQEEWQHLDPSQRALYRDVMLENYSNLVSVGYCVHKPEVIFRLQQGEEPWKQEEEFPSQSFPEVWTADHLKERSQENQSKHL

SEQ ID NO: 69 - protein, 98 aa, synthetic construct
MTKSKEAVTFKDVAVVFSEEELQLLDLAQRKLYRDVMLENFRNVVSVGHQSTPDGLPQLEREEKLWMMKMATQRDNSSGAKNLKEMETLQEVGLRYLP

SEQ ID NO: 70 - protein, 112 aa, synthetic construct
MSQKPQVLGPEKQDGSCEASVSFEDVTVDFSREEWQQLDPAQRCLYRDVMLELYSHLFAVGYHIPNPEVIFRMLKEKEPRVEEAEVSHQRCQEREFGLEIPQKEISKKASFQ

SEQ ID NO: 71 - protein, 95 aa, synthetic construct
MELVTFRDVAIEFSPEEWKCLDPAQQNLYRDVMLENYRNLVSLGFVISNPDLVTCLEQIKEPCNLKIHETAAKPPAICSPFSQDLSPVQGIEDSF

SEQ ID NO: 72 - protein, 112 aa, synthetic construct
MSTLLQGGHNLLSSASFQESVTFKDVIVDFTQEEWKQLDPGQRDLFRDVTLENYTHLVSIGLQVSKPDVISQLEQGTEPWIMEPSIPVGTCADWETRLENSVSAPEPDISEE

SEQ ID NO: 73 - protein, 112 aa, synthetic construct
MDPAQVPVAADLLTDHEEGYVTFEDVAVYFSQEEWRLLDDAQRLLYRNVMLENFTLLASLGLASSKTHEITQLESWEEPFMPAWEVVTSAIPRGCWHGAEAEEAPEQIASVG

SEQ ID NO: 74 - protein, 112 aa, synthetic construct
MKKLEAVGTGIEPKAMSQGLVTFGDVAVDFSQEEWEWLNPIQRNLYRKVMLENYRNLASLGLCVSKPDVISSLEQGKEPWTVKRKMTRAWCPDLKAVWKIKELPLKKDFCEG

SEQ ID NO: 75 - protein, 108 aa, synthetic construct
MSLLGEHWDYDALFETQPGLVTIKNLAVDFRQQLHPAQKNFCKNGIWENNSDLGSAGHCVAKPDLVSLLEQEKEPWMVKRELTGSLFSGQRSVHETQELFPKQDSYAE

SEQ ID NO: 76 - protein, 113 aa, synthetic construct
MLALAASQPARLEERLIRDRDLGASLLPAAPQEQWRQLDSTQKEQYWDLILETYGKMVSGAGISHPKSDLTNSIEFGEELAGIYLHVNEKIPRPTCIGDRQENDKENLNLENH

SEQ ID NO: 77 - protein, 131 aa, synthetic construct
MEGRPGETTDTCFVSGVHGPVALGDIPFYFSREEWGTLDPAQRDLFWDIKRENSRNTTLGFGLKGQSEKSLLQEMVPVVPGQTGSDVTVSWSPEEAEAWESENRPRAALGPVVGARRGRPPTRRRQFRDLA

SEQ ID NO: 78 - protein, 116 aa, synthetic construct
MVAVVRALQRALDGTSSQGMVTFEDTAVSLTWEEWERLDPARRDFCRESAQKDSGSTVPPSLESRVENKELIPMQQILEEAEPQGQLQEAFQGKRPLFSKCGSTHEDRVEKQSGDP

SEQ ID NO: 79 - protein, 113 aa, synthetic construct
MNKSQGSVSFTDVTVDFTQEEWEQLDPSQRILYMDVMLENYSNLLSVEVWKADDQMERDHRNPDEQARQFLILKNQTPIEERGDLFGKALNLNTDFVSLRQVPYKYDLYEKTL

SEQ ID NO: 80 - protein, 108 aa, synthetic construct
MAHGSVTFRDVAIDFSQEEWEFLDPAQRDLYRDVMWENYSNFISLGPSISKPDVITLLDEERKEPGMVVREGTRRYCPDLESRYRTNTLSPEKDIYEIYSFQWDIMER

SEQ ID NO: 81 - protein, 141 aa, synthetic construct
MAAAVLRDSTSVPVTAEAKLMGFTQGCVTFEDVAIYFSQEEWGLLDEAQRLLYRDVMLENFALITALVCWHGMEDEETPEQSVSVEGVPQVRTPEASPSTQKIQSCDMCVPFLTDILHLTDLPGQELYLTGACAVFHQDQK

SEQ ID NO: 82 - protein, 133 aa, synthetic construct
MLPPTAASQREGHTEGGELVNELLKSWLKGLVTFEDVAVEFTQEEWALLDPAQRTLYRDVMLENCRNLASLGNQVDKPRLISQLEQEDKVMTEERGILSGTCPDVENPFKAKGLTPKLHVFRKEQSRNMKMER

SEQ ID NO: 83 - protein, 107 aa, synthetic construct
MAQESVMFSDVSVDFSQEEWECLNDDQRDLYRDVMLENYSNLVSMGHSISKPNVISYLEQGKEPWLADRELTRGQWPVLESRCETKKLFLKKEIYEIESTQWEIMEK

SEQ ID NO: 84 - protein, 114 aa, synthetic construct
MPGAPGSLEMGPLTFRDVTIEFSLEEWQCLDTVQQNLYRDVMLENYRNLVFLGMAVFKPDLITCLKQGKEPWNMKRHEMVTKPPVMRSHFTQDLWPDQSTKDSFQEVILRTYAR

SEQ ID NO: 85 - protein, 95 aa, synthetic construct
MAGSQFPDFKHLGTFLVFEELVTFEDVLVDFSPEELSSLSAAQRNLYREVMLENYRNLVSLGHQFSKPDIISRLEEEESYAMETDSRHTVICQGE

SEQ ID NO: 86 - protein, 78 aa, synthetic construct
MPGPPRSLEMGLLTFRDVAIEFSLEEWQHLDIAQQNLYRNVMLENYRNLAFLGIAVSKPDLITCLEQGKEPWNMKRHE

SEQ ID NO: 87 - protein, 93 aa, synthetic construct
MAPPLAPLPPRDPNGAGPEWREPGAVSFADVAVYFCREEWGCLRPAQRALYRDVMRETYGHLSALGIGGNKPALISWVEEEAELWGPAAQDPE

SEQ ID NO: 88 - protein, 100 aa, synthetic construct
MGPPLAPRPAHVPGEAGPRRTRESRPGAVSFADVAVYFSPEEWECLRPAQRALYRDVMRETFGHLGALGFSVPKPAFISWVEGEVEAWSPEAQDPDGESS

SEQ ID NO: 89 - protein, 573 aa, synthetic construct
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLWYLSLEEVWKCRDQLDKYQENPERHLRQVAFTQKKVLTQERVSESGKYGGNCLLPAQLVLREYFHKRDSHTKSLKHDLVLNGHQDSCASNSNECGQTFCQNIHLIQFARTHTGDKSYKCPDNDNSLTHGSSLGISKGIHREKPYECKECGKFFSWRSNLTRHQLIHTGEKPYECKECGKSFSRSSHLIGHQKTHTGEEPYECKECGKSFSWFSHLVTHQRTHTGDKLYTCNQCGKSFVHSSRLIRHQRTHTGEKPYECPECGKSFRQSTHLILHQRTHVRVRPYECNECGKSYSQRSHLVVHHRIHTGLKPFECKDCGKCFSRSSHLYSHQRTHTGEKPYECHDCGKSFSQSSALIVHQRIHTGEKPYECCQCGKAFIRKNDLIKHQRIHVGEETYKCNQCGIIFSQNSPFIVHQIAHTGEQFLTCNQCGTALVNTSNLIGYQTNHIRENAY

SEQ ID NO: 90 - protein, 191 aa, synthetic construct
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 91 - protein, 228 aa, synthetic construct
MTMGDKKSPTRPKRQAKPAADEGFWDCSVCTFRNSAEAFKCSICDVRKGTSTRKPRINSQLVAQQVAQQYATPPPPKKEKKEKVEKQDKEKPEKDKEISPSVTKKNTNKKTKPKSDILKDPPSEANSIQSANATTKTSETNHTSRPRLKNVDRSTAQQLAVTVGNVTVIITDFKEKTRSSSTSSSTVTSSAGSEQQNQSSSGSESTDKGSSRSSTPKGDMSAVNDESF

SEQ ID NO: 92 - protein, 180 aa, synthetic construct
MGDKKSPTRPKRQPKPSSDEGYWDCSVCTFRNSAEAFKCMMCDVRKGTSTRKPRPVSQLVAQQVTQQFVPPTQSKKEKKDKVEKEKSEKETTSKKNSHKKTRPRLKNVDRSSAQHLEVTVGDLTVIITDFKEKTKSPPASSAASADQHSQSGSSSDNTERGMSRSSSPRGEASSLNGESH

SEQ ID NO: 93 - protein, 3065 aa, synthetic construct
MEEKQQIILANQDGGTVAGAAPTFFVILKQPGNGKTDQGILVTNQDACALASSVSSPVKSKGKICLPADCTVGGITVTLDNNSMWNEFYHRSTEMILTKQGRRMFPYCRYWITGLDSNLKYILVMDISPVDNHRYKWNGRWWEPSGKAEPHVLGRVFIHPESPSTGHYWMHQPVSFYKLKLTNNTLDQEGHIILHSMHRYLPRLHLVPAEKAVEVIQLNGPGVHTFTFPQTEFFAVTAYQNIQITQLKIDYNPFAKGFRDDGLNNKPQRDGKQKNSSDQEGNNISSSSGHRVRLTEGQGSEIQPGDLDPLSRGHETSGKGLEKTSLNIKRDFLGFMDTDSALSEVPQLKQEISECLIASSFEDDSRVASPLDQNGSFNVVIKEEPLDDYDYELGECPEGVTVKQEETDEETDVYSNSDDDPILEKQLKRHNKVDNPEADHLSSKWLPSSPSGVAKAKMFKLDTGKMPVVYLEPCAVTRSTVKISELPDNMLSTSRKDKSSMLAELEYLPTYIENSNETAFCLGKESENGLRKHSPDLRVVQKYPLLKEPQWKYPDISDSISTERILDDSKDSVGDSLSGKEDLGRKRTTMLKIATAAKVVNANQNASPNVPGKRGRPRKLKLCKAGRPPKNTGKSLISTKNTPVSPGSTFPDVKPDLEDVDGVLFVSFESKEALDIHAVDGTTEESSSLQASTTNDSGYRARISQLEKELIEDLKTLRHKQVIHPGLQEVGLKLNSVDPTMSIDLKYLGVQLPLAPATSFPFWNLTGTNPASPDAGFPFVSRTGKTNDFTKIKGWRGKFHSASASRNEGGNSESSLKNRSAFCSDKLDEYLENEGKLMETSMGFSSNAPTSPVVYQLPTKSTSYVRTLDSVLKKQSTISPSTSYSLKPHSVPPVSRKAKSQNRQATFSGRTKSSYKSILPYPVSPKQKYSHVILGDKVTKNSSGIISENQANNFVVPTLDENIFPKQISLRQAQQQQQQQQGSRPPGLSKSQVKLMDLEDCALWEGKPRTYITEERADVSLTTLLTAQASLKTKPIHTIIRKRAPPCNNDFCRLGCVCSSLALEKRQPAHCRRPDCMFGCTCLKRKVVLVKGGSKTKHFQRKAAHRDPVFYDTLGEEAREEEEGIREEEEQLKEKKKRKKLEYTICETEPEQPVRHYPLWVKVEGEVDPEPVYIPTPSVIEPMKPLLLPQPEVLSPTVKGKLLTGIKSPRSYTPKPNPVIREEDKDPVYLYFESMMTCARVRVYERKKEDQRQPSSSSSPSPSFQQQTSCHSSPENHNNAKEPDSEQQPLKQLTCDLEDDSDKLQEKSWKSSCNEGESSSTSYMHQRSPGGPTKLIEIISDCNWEEDRNKILSILSQHINSNMPQSLKVGSFIIELASQRKSRGEKNPPVYSSRVKISMPSCQDQDDMAEKSGSETPDGPLSPGKMEDISPVQTDALDSVRERLHGGKGLPFYAGLSPAGKLVAYKRKPSSSTSGLIQVASNAKVAASRKPRTLLPSTSNSKMASSSGTATNRPGKNLKAFVPAKRPIAARPSPGGVFTQFVMSKVGALQQKIPGVSTPQTLAGTQKFSIRPSPVMVVTPVVSSEPVQVCSPVTAAVTTTTPQVFLENTTAVTPMTAISDVETKETTYSSGATTTGVVEVSETNTSTSVTSTQSTATVNLTKTTGITTPVASVAFPKSLVASPSTITLPVASTASTSLVVVTAAASSSMVTTPTSSLGSVPIILSGINGSPPVSQRPENAAQIPVATPQVSPNTVKRAGPRLLLIPVQQGSPTLRPVSNTQLQGHRMVLQPVRSPSGMNLFRHPNGQIVQLLPLHQLRGSNTQPNLQPVMFRNPGSVMGIRLPAPSKPSETPPSSTSSSAFSVMNPVIQAVGSSSAVNVITQAPSLLSSGASFVSQAGTLTLRISPPEPQSFASKTGSETKITYSSGGQPVGTASLIPLQSGSFALLQLPGQKPVPSSILQHVASLQMKRESQNPDQKDETNSIKREQETKKVLQSEGEAVDPEANVIKQNSGAATSEETLNDSLEDRGDHLDEECLPEEGCATVKPSEHSCITGSHTDQDYKDVNEEYGARNRKSSKEKVAVLEVRTISEKASNKTVQNLSKVQHQKLGDVKVEQQKGFDNPEENSSEFPVTFKEESKFELSGSKVMEQQSNLQPEAKEKECGDSLEKDRERWRKHLKGPLTRKCVGASQECKKEADEQLIKETKTCQENSDVFQQEQGISDLLGKSGITEDARVLKTECDSWSRISNPSAFSIVPRRAAKSSRGNGHFQGHLLLPGEQIQPKQEKKGGRSSADFTVLDLEEDDEDDNEKTDDSIDEIVDVVSDYQSEEVDDVEKNNCVEYIEDDEEHVDIETVEELSEEINVAHLKTTAAHTQSFKQPSCTHISADEKAAERSRKAPPIPLKLKPDYWSDKLQKEAEAFAYYRRTHTANERRRRGEMRDLFEKLKITLGLLHSSKVSKSLILTRAFSEIQGLTDQADKLIGQKNLLTRKRNILIRKVSSLSGKTEEVVLKKLEYIYAKQQALEAQKRKKKMGSDEFDISPRISKQQEGSSASSVDLGQMFINNRRGKPLILSRKKDQATENTSPLNTPHTSANLVMTPQGQLLTLKGPLFSGPVVAVSPDLLESDLKPQVAGSAVALPENDDLFMMPRIVNVTSLATEGGLVDMGGSKYPHEVPDSKPSDHLKDTVRNEDNSLEDKGRISSRGNRDGRVTLGPTQVFLANKDSGYPQIVDVSNMQKAQEFLPKKISGDMRGIQYKWKESESRGERVKSKDSSFHKLKMKDLKDSSIEMELRKVTSAIEEAALDSSELLTNMEDEDDTDETLTSLLNEIAFLNQQLNDDSVGLAELPSSMDTEFPGDARRAFISKVPPGSRATFQVEHLGTGLKELPDVQGESDSISPLLLHLEDDDFSENEKQLAEPASEPDVLKIVIDSEIKDSLLSNKKAIDGGKNTSGLPAEPESVSSPPTLHMKTGLENSNSTDTLWRPMPKLAPLGLKVANPSSDADGQSLKVMPCLAPIAAKVGSVGHKMNLTGNDQEGRESKVMPTLAPVVAKLGNSGASPSSAGK

SEQ ID NO: 94 - protein, 185 aa, synthetic construct
MGKKQNKKKVEEVLEEEEEEYVVEKVLDRRVVKGKVEYLLKWKGFSDEDNTWEPEENLDCPDLIAEFLQSQKTAHETDKSEGGKRKADSDSEDKGEESKPKKKKEESEKPRGFARGLEPERIIGATDSSGELMFLMKWKNSDEADLVPAKEANVKCPQVVISFYEERLTWHSYPSEDDDKKDDKN

SEQ ID NO: 95 - protein, 660 aa, synthetic construct
MLVCYSVLACEILWDLPCSIMGSPLGHFTWDKYLKETCSVPAPVHCFKQSYTPPSNEFKISMKLEAQDPRNTTSTCIATVVGLTGARLRLRLDGSDNKNDFWRLVDSAEIQPIGNCEKNGGMLQPPLGFRLNASSWPMFLLKTLNGAEMAPIRIFHKEPPSPSHNFFKMGMKLEAVDRKNPHFICPATIGEVRGSEVLVTFDGWRGAFDYWCRFDSRDIFPVGWCSLTGDNLQPPGTKVVIPKNPYPASDVNTEKPSIHSSTKTVLEHQPGQRGRKPGKKRGRTPKTLISHPISAPSKTAEPLKFPKKRGPKPGSKRKPRTLLNPPPASPTTSTPEPDTSTVPQDAATIPSSAMQAPTVCIYLNKNGSTGPHLDKKKVQQLPDHFGPARASVVLQQAVQACIDCAYHQKTVFSFLKQGHGGEVISAVFDREQHTLNLPAVNSITYVLRFLEKLCHNLRSDNLFGNQPFTQTHLSLTAIEYSHSHDRYLPGETFVLGNSLARSLEPHSDSMDSASNPTNLVSTSQRHRPLLSSCGLPPSTASAVRRLCSRGVLKGSNERRDMESFWKLNRSPGSDRYLESRDASRLSGRDPSSWTVEDVMQFVREADPQLGPHADLFRKHEIDGKALLLLRSDMMMKYMGLKLGPALKLSYHIDRLKQGKF

SEQ ID NO: 96 - protein, 860 aa, synthetic construct
MEQVAEGARVTAVPVSAADSTEELAEVEEGVGVVGEDNDAAARGAEAFGDSEEDGEDVFEVEKILDMKTEGGKVLYKVRWKGYTSDDDTWEPEIHLEDCKEVLLEFRKKIAENKAKAVRKDIQRLSLNNDIFEANSDSDQQSETKEDTSPKKKKKKLRQREEKSPDDLKKKKAKAGKLKDKSKPDLESSLESLVFDLRTKKRISEAKEELKESKKPKKDEVKETKELKKVKKGEIRDLKTKTREDPKENRKTKKEKFVESQVESESSVLNDSPFPEDDSEGLHSDSREEKQNTKSARERAGQDMGLEHGFEKPLDSAMSAEEDTDVRGRRKKKTPRKAEDTRENRKLENKNAFLEKKTVPKKQRNQDRSKSAAELEKLMPVSAQTPKGRRLSGEERGLWSTDSAEEDKETKRNESKEKYQKRHDSDKEEKGRKEPKGLKTLKEIRNAFDLFKLTPEEKNDVSENNRKREEIPLDFKTIDDHKTKENKQSLKERRNTRDETDTWAYIAAEGDQEVLDSVCQADENSDGRQQILSLGMDLQLEWMKLEDFQKHLDGKDENFAATDAIPSNVLRDAVKNGDYITVKVALNSNEEYNLDQEDSSGMTLVMLAAAGGQDDLLRLLITKGAKVNGRQKNGTTALIHAAEKNFLTTVAILLEAGAFVNVQQSNGETALMKACKRGNSDIVRLVIECGADCNILSKHQNSALHFAKQSNNVLVYDLLKNHLETLSRVAEETIKDYFEARLALLEPVFPIACHRLCEGPDFSTDFNYKPPQNIPEGSGILLFIFHANFLGKEVIARLCGPCSVQAVVLNDKFQLPVFLDSHFVYSFSPVAGPNKLFIRLTEAPSAKVKLLIGAYRVQLQ

SEQ ID NO: 97 - protein, 103 aa, synthetic construct
MSEEKPKEGVKTENDHINLKVAGQDGSVVQFKIKRHTPLSKLMKAYCERQGLSMRQIRFRFDGQPINETDTPAQLEMEDEDTIDVFQQQTGGVPESSLAGHSF

SEQ ID NO: 98 - protein, 4834 aa, synthetic construct
MPSESFCLAAQARLDSKWLKTDIQLAFTRDGLCGLWNEMVKDGEIVYTGTESTQNGELPPRKDDSVEPSGTKKEDLNDKEKKDEEETPAPIYRAKSILDSWVWGKQPDVNELKECLSVLVKEQQALAVQSATTTLSALRLKQRLVILERYFIALNRTVFQENVKVKWKSSGISLPPVDKKSSRPAGKGVEGLARVGSRAALSFAFAFLRRAWRSGEDADLCSELLQESLDALRALPEASLFDESTVSSVWLEVVERATRFLRSVVTGDVHGTPATKGPGSIPLQDQHLALAILLELAVQRGTLSQMLSAILLLLQLWDSGAQETDNERSAQGTSAPLLPLLQRFQSIICRKDAPHSEGDMHLLSGPLSPNESFLRYLTLPQDNELAIDLRQTAVVVMAHLDRLATPCMPPLCSSPTSHKGSLQEVIGWGLIGWKYYANVIGPIQCEGLANLGVTQIACAEKRFLILSRNGRVYTQAYNSDTLAPQLVQGLASRNIVKIAAHSDGHHYLALAATGEVYSWGCGDGGRLGHGDTVPLEEPKVISAFSGKQAGKHVVHIACGSTYSAAITAEGELYTWGRGNYGRLGHGSSEDEAIPMLVAGLKGLKVIDVACGSGDAQTLAVTENGQVWSWGDGDYGKLGRGGSDGCKTPKLIEKLQDLDVVKVRCGSQFSIALTKDGQVYSWGKGDNQRLGHGTEEHVRYPKLLEGLQGKKVIDVAAGSTHCLALTEDSEVHSWGSNDQCQHFDTLRVTKPEPAALPGLDTKHIVGIACGPAQSFAWSSCSEWSIGLRVPFVVDICSMTFEQLDLLLRQVSEGMDGSADWPPPQEKECVAVATLNLLRLQLHAAISHQVDPEFLGLGLGSILLNSLKQTVVTLASSAGVLSTVQSAAQAVLQSGWSVLLPTAEERARALSALLPCAVSGNEVNISPGRRFMIDLLVGSLMADGGLESALHAAITAEIQDIEAKKEAQKEKEIDEQEANASTFHRSRTPLDKDLINTGICESSGKQCLPLVQLIQQLLRNIASQTVARLKDVARRISSCLDFEQHSRERSASLDLLLRFQRLLISKLYPGESIGQTSDISSPELMGVGSLLKKYTALLCTHIGDILPVAASIASTSWRHFAEVAYIVEGDFTGVLLPELVVSIVLLLSKNAGLMQEAGAVPLLGGLLEHLDRFNHLAPGKERDDHEELAWPGIMESFFTGQNCRNNEEVTLIRKADLENHNKDGGFWTVIDGKVYDIKDFQTQSLTGNSILAQFAGEDPVVALEAALQFEDTRESMHAFCVGQYLEPDQEIVTIPDLGSLSSPLIDTERNLGLLLGLHASYLAMSTPLSPVEIECAKWLQSSIFSGGLQTSQIHYSYNEEKDEDHCSSPGGTPASKSRLCSHRRALGDHSQAFLQAIADNNIQDHNVKDFLCQIERYCRQCHLTTPIMFPPEHPVEEVGRLLLCCLLKHEDLGHVALSLVHAGALGIEQVKHRTLPKSVVDVCRVVYQAKCSLIKTHQEQGRSYKEVCAPVIERLRFLFNELRPAVCNDLSIMSKFKLLSSLPRWRRIAQKIIRERRKKRVPKKPESTDDEEKIGNEESDLEEACILPHSPINVDKRPIAIKSPKDKWQPLLSTVTGVHKYKWLKQNVQGLYPQSPLLSTIAEFALKEEPVDVEKMRKCLLKQLERAEVRLEGIDTILKLASKNFLLPSVQYAMFCGWQRLIPEGIDIGEPLTDCLKDVDLIPPFNRMLLEVTFGKLYAWAVQNIRNVLMDASAKFKELGIQPVPLQTITNENPSGPSLGTIPQARFLLVMLSMLTLQHGANNLDLLLNSGMLALTQTALRLIGPSCDNVEEDMNASAQGASATVLEETRKETAPVQLPVSGPELAAMMKIGTRVMRGVDWKWGDQDGPPPGLGRVIGELGEDGWIRVQWDTGSTNSYRMGKEGKYDLKLAELPAAAQPSAEDSDTEDDSEAEQTERNIHPTAMMFTSTINLLQTLCLSAGVHAEIMQSEATKTLCGLLRMLVESGTTDKTSSPNRLVYREQHRSWCTLGFVRSIALTPQVCGALSSPQWITLLMKVVEGHAPFTATSLQRQILAVHLLQAVLPSWDKTERARDMKCLVEKLFDFLGSLLTTCSSDVPLLRESTLRRRRVRPQASLTATHSSTLAEEVVALLRTLHSLTQWNGLINKYINSQLRSITHSFVGRPSEGAQLEDYFPDSENPEVGGLMAVLAVIGGIDGRLRLGGQVMHDEFGEGTVTRITPKGKITVQFSDMRTCRVCPLNQLKPLPAVAFNVNNLPFTEPMLSVWAQLVNLAGSKLEKHKIKKSTKQAFAGQVDLDLLRCQQLKLYILKAGRALLSHQDKLRQILSQPAVQETGTVHTDDGAVVSPDLGDMSPEGPQPPMILLQQLLASATQPSPVKAIFDKQELEAAALAVCQCLAVESTHPSSPGFEDCSSSEATTPVAVQHIRPARVKRRKQSPVPALPIVVQLMEMGFSRRNIEFALKSLTGASGNASSLPGVEALVGWLLDHSDIQVTELSDADTVSDEYSDEEVVEDVDDAAYSMSTGAVVTESQTYKKRADFLSNDDYAVYVRENIQVGMMVRCCRAYEEVCEGDVGKVIKLDRDGLHDLNVQCDWQQKGGTYWVRYIHVELIGYPPPSSSSHIKIGDKVRVKASVTTPKYKWGSVTHQSVGVVKAFSANGKDIIVDFPQQSHWTGLLSEMELVPSIHPGVTCDGCQMFPINGSRFKCRNCDDFDFCETCFKTKKHNTRHTFGRINEPGQSAVFCGRSGKQLKRCHSSQPGMLLDSWSRMVKSLNVSSSVNQASRLIDGSEPCWQSSGSQGKHWIRLEIFPDVLVHRLKMIVDPADSSYMPSLVVVSGGNSLNNLIELKTININPSDTTVPLLNDCTEYHRYIEIAIKQCRSSGIDCKIHGLILLGRIRAEEEDLAAVPFLASDNEEEEDEKGNSGSLIRKKAAGLESAATIRTKVFVWGLNDKDQLGGLKGSKIKVPSFSETLSALNVVQVAGGSKSLFAVTVEGKVYACGEATNGRLGLGISSGTVPIPRQITALSSYVVKKVAVHSGGRHATALTVDGKVFSWGEGDDGKLGHFSRMNCDKPRLIEALKTKRIRDIACGSSHSAALTSSGELYTWGLGEYGRLGHGDNTTQLKPKMVKVLLGHRVIQVACGSRDAQTLALTDEGLVFSWGDGDFGKLGRGGSEGCNIPQNIERLNGQGVCQIECGAQFSLALTKSGVVWTWGKGDYFRLGHGSDVHVRKPQVVEGLRGKKIVHVAVGALHCLAVTDSGQVYAWGDNDHGQQGNGTTTVNRKPTLVQGLEGQKITRVACGSSHSVAWTTVDVATPSVHEPVLFQTARDPLGASYLGVPSDADSSAASNKISGASNSKPNRPSLAKILLSLDGNLAKQQALSHILTALQIMYARDAVVGALMPAAMIAPVECPSFSSAAPSDASAMASPMNGEECMLAVDIEDRLSPNPWQEKREIVSSEDAVTPSAVTPSAPSASARPFIPVTDDLGAASIIAETMTKTKEDVESQNKAAGPEPQALDEFTSLLIADDTRVVVDLLKLSVCSRAGDRGRDVLSAVLSGMGTAYPQVADMLLELCVTELEDVATDSQSGRLSSQPVVVESSHPYTDDTSTSGTVKIPGAEGLRVEFDRQCSTERRHDPLTVMDGVNRIVSVRSGREWSDWSSELRIPGDELKWKFISDGSVNGWGWRFTVYPIMPAAGPKELLSDRCVLSCPSMDLVTCLLDFRLNLASNRSIVPRLAASLAACAQLSALAASHRMWALQRLRKLLTTEFGQSININRLLGENDGETRALSFTGSALAALVKGLPEALQRQFEYEDPIVRGGKQLLHSPFFKVLVALACDLELDTLPCCAETHKWAWFRRYCMASRVAVALDKRTPLPRLFLDEVAKKIRELMADSENMDVLHESHDIFKREQDEQLVQWMNRRPDDWTLSAGGSGTIYGWGHNHRGQLGGIEGAKVKVPTPCEALATLRPVQLIGGEQTLFAVTADGKLYATGYGAGGRLGIGGTESVSTPTLLESIQHVFIKKVAVNSGGKHCLALSSEGEVYSWGEAEDGKLGHGNRSPCDRPRVIESLRGIEVVDVAAGGAHSACVTAAGDLYTWGKGRYGRLGHSDSEDQLKPKLVEALQGHRVVDIACGSGDAQTLCLTDDDTVWSWGDGDYGKLGRGGSDGCKVPMKIDSLTGLGVVKVECGSQFSVALTKSGAVYTWGKGDYHRLGHGSDDHVRRPRQVQGLQGKKVIAIATGSLHCVCCTEDGEVYTWGDNDEGQLGDGTTNAIQRPRLVAALQGKKVNRVACGSAHTLAWSTSKPASAGKLPAQVPMEYNHLQEIPIIALRNRLLLLHHLSELFCPCIPMFDLEGSLDETGLGPSVGFDTLRGILISQGKEAAFRKVVQATMVRDRQHGPVVELNRIQVKRSRSKGGLAGPDGTKSVFGQMCAKMSSFGPDSLLLPHRVWKVKFVGESVDDCGGGYSESIAEICEELQNGLTPLLIVTPNGRDESGANRDCYLLSPAARAPVHSSMFRFLGVLLGIAIRTGSPLSLNLAEPVWKQLAGMSLTIADLSEVDKDFIPGLMYIRDNEATSEEFEAMSLPFTVPSASGQDIQLSSKHTHITLDNRAEYVRLAINYRLHEFDEQVAAVREGMARVVPVPLLSLFTGYELETMVCGSPDIPLHLLKSVATYKGIEPSASLIQWFWEVMESFSNTERSLFLRFVWGRTRLPRTIADFRGRDFVIQVLDKYNPPDHFLPESYTCFFLLKLPRYSCKQVLEEKLKYAIHFCKSIDTDDYARIALTGEPAADDSSDDSDNEDVDSFASDSTQDYLTGH

SEQ ID NO: 99 - protein, 593 aa, synthetic construct
MAEMGSKGVTAGKIASNVQKKLTRAQEKVLQKLGKADETKDEQFEQCVQNFNKQLTEGTRLQKDLRTYLASVKAMHEASKKLNECLQEVYEPDWPGRDEANKIAENNDLLWMDYHQKLVDQALLTMDTYLGQFPDIKSRIAKRGRKLVDYDSARHHYESLQTAKKKDEAKIAKPVSLLEKAAPQWCQGKLQAHLVAQTNLLRNQAEEELIKAQKVFEEMNVDLQEELPSLWNSRVGFYVNTFQSIAGLEENFHKEMSKLNQNLNDVLVGLEKQHGSNTFTVKAQPSDNAPAKGNKSPSPPDGSPAATPEIRVNHEPEPAGGATPGATLPKSPSQLRKGPPVPPPPKHTPSKEVKQEQILSLFEDTFVPEISVTTPSQFEAPGPFSEQASLLDLDFDPLPPVTSPVKAPTPSGQSIPWDLWEPTESPAGSLPSGEPSAAEGTFAVSWPSQTAEPGPAQPAEASEVAGGTQPAAGAQEPGETAASEAASSSLPAVVVETFPATVNGTVEGGSGAGRLDLPPGFMFKVQAQHDYTATDTDELQLKAGDVVLVIPFQNPEEQDEGWLMGVKESDWNQHKELEKCRGVFPENFTERVP

SEQ ID NO: 100 - protein, 344 aa, synthetic construct
MHRTTRIKITELNPHLMCALCGGYFIDATTIVECLHSFCKTCIVRYLETNKYCPMCDVQVHKTRPLLSIRSDKTLQDIVYKLVPGLFKDEMKRRRDFYAAYPLTEVPNGSNEDRGEVLEQEKGALSDDEIVSLSIEFYEGARDRDEKKGPLENGDGDKEKTGVRFLRCPAAMTVMHLAKFLRNKMDVPSKYKVEVLYEDEPLKEYYTLMDIAYIYPWRRNGPLPLKYRVQPACKRLTLATVPTPSEGTNTSGASECESVSDKAPSPATLPATSSSLPSPATPSHGSPSSHGPPATHPTSPTPPSTASGATTAANGGSLNCLQTPSSTSRGRKMTVNGAPVPPLT

SEQ ID NO: 101 - protein, 526 aa, synthetic construct
MDVRFYPPPAQPAAAPDAPCLGPSPCLDPYYCNKFDGENMYMSMTEPSQDYVPASQSYPGPSLESEDFNIPPITPPSLPDHSLVHLNEVESGYHSLCHPMNHNGLLPFHPQNMDLPEITVSNMLGQDGTLLSNSISVMPDIRNPEGTQYSSHPQMAAMRPRGQPADIRQQPGMMPHGQLTTINQSQLSAQLGLNMGGSNVPHNSPSPPGSKSATPSPSSSVHEDEGDDTSKINGGEKRPASDMGKKPKTPKKKKKKDPNEPQKPVSAYALFFRDTQAAIKGQNPNATFGEVSKIVASMWDGLGEEQKQVYKKKTEAAKKEYLKQLAAYRASLVSKSYSEPVDVKTSQPPQLINSKPSVFHGPSQAHSALYLSSHYHQQPGMNPHLTAMHPSLPRNIAPKPNNQMPVTVSIANMAVSPPPPLQISPPLHQHLNMQQHQPLTMQQPLGNQLPMQVQSALHSPTMQQGFTLQPDYQTIINPTSTAAQVVTQAMEYVRSGCRNPPPQPVDWNNDYCSSGGMQRDKALYLT

SEQ ID NO: 102 - protein, 472 aa, synthetic construct
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 103 - protein, 457 aa, synthetic construct
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 104 - protein, 584 aa, synthetic construct
MASVQASRRQWCYLCDLPKMPWAMVWDFSEAVCRGCVNFEGADRIELLIDAARQLKRSHVLPEGRSPGPPALKHPATKDLAAAAAQGPQLPPPQAQPQPSGTGGGVSGQDRYDRATSSGRLPLPSPALEYTLGSRLANGLGREEAVAEGARRALLGSMPGLMPPGLLAAAVSGLGSRGLTLAPGLSPARPLFGSDFEKEKQQRNADCLAELNEAMRGRAEEWHGRPKAVREQLLALSACAPFNVRFKKDHGLVGRVFAFDATARPPGYEFELKLFTEYPCGSGNVYAGVLAVARQMFHDALREPGKALASSGFKYLEYERRHGSGEWRQLGELLTDGVRSFREPAPAEALPQQYPEPAPAALCGPPPRAPSRNLAPTPRRRKASPEPEGEAAGKMTTEEQQQRHWVAPGGPYSAETPGVPSPIAALKNVAEALGHSPKDPGGGGGPVRAGGASPAASSTAQPPTQHRLVARNGEAEVSPTAGAEAVSGGGSGTGATPGAPLCCTLCRERLEDTHFVQCPSVPGHKFCFPCSREFIKAQGPAGEVYCPSGDKCPLVGSSVPWAFMQGEIATILAGDIKVKKERDP

SEQ ID NO: 105 - protein, 587 aa, synthetic construct
MAAAVAVAAASRRQSCYLCDLPRMPWAMIWDFTEPVCRGCVNYEGADRVEFVIETARQLKRAHGCFPEGRSPPGAAASAAAKPPPLSAKDILLQQQQQLGHGGPEAAPRAPQALERYPLAAAAERPPRLGSDFGSSRPAASLAQPPTPQPPPVNGILVPNGFSKLEEPPELNRQSPNPRRGHAVPPTLVPLMNGSATPLPTALGLGGRAAASLAAVSGTAAASLGSAQPTDLGAHKRPASVSSSAAVEHEQREAAAKEKQPPPPAHRGPADSLSTAAGAAELSAEGAGKSRGSGEQDWVNRPKTVRDTLLALHQHGHSGPFESKFKKEPALTAGRLLGFEANGANGSKAVARTARKRKPSPEPEGEVGPPKINGEAQPWLSTSTEGLKIPMTPTSSFVSPPPPTASPHSNRTTPPEAAQNGQSPMAALILVADNAGGSHASKDANQVHSTTRRNSNSPPSPSSMNQRRLGPREVGGQGAGNTGGLEPVHPASLPDSSLATSAPLCCTLCHERLEDTHFVQCPSVPSHKFCFPCSRQSIKQQGASGEVYCPSGEKCPLVGSNVPWAFMQGEIATILAGDVKVKKERDS

SEQ ID NO: 106 - protein, 796 aa, synthetic construct
MSAAQVSSSRRQSCYLCDLPRMPWAMIWDFSEPVCRGCVNYEGADRIEFVIETARQLKRAHGCFQDGRSPGPPPPVGVKTVALSAKEAAAAAAAAAAAAAAAQQQQQQQQQQQQQQQQQQQQQQQQQLNHVDGSSKPAVLAAPSGLERYGLSAAAAAAAAAAAAVEQRSRFEYPPPPVSLGSSSHTARLPNGLGGPNGFPKPTPEEGPPELNRQSPNSSSAAASVASRRGTHGGLVTGLPNPGGGGGPQLTVPPNLLPQTLLNGPASAAVLPPPPPHALGSRGPPTPAPPGAPGGPACLGGTPGVSATSSSASSSTSSSVAEVGVGAGGKRPGSVSSTDQERELKEKQRNAEALAELSESLRNRAEEWASKPKMVRDTLLTLAGCTPYEVRFKKDHSLLGRVFAFDAVSKPGMDYELKLFIEYPTGSGNVYSSASGVAKQMYQDCMKDFGRGLSSGFKYLEYEKKHGSGDWRLLGDLLPEAVRFFKEGVPGADMLPQPYLDASCPMLPTALVSLSRAPSAPPGTGALPPAAPSGRGAAASLRKRKASPEPPDSAEGALKLGEEQQRQQWMANQSEALKLTMSAGGFAAPGHAAGGPPPPPPPLGPHSNRTTPPESAPQNGPSPMAALMSVADTLGTAHSPKDGSSVHSTTASARRNSSSPVSPASVPGQRRLASRNGDLNLQVAPPPPSAHPGMDQVHPQNIPDSPMANSGPLCCTICHERLEDTHFVQCPSVPSHKFCFPCSRESIKAQGATGEVYCPSGEKCPLVGSNVPWAFMQGEIATILAGDVKVKKERDP

SEQ ID NO: 107 - protein, 388 aa, synthetic construct
MTASVLLHPRWIEPTVMFLYDNGGGLVADELNKNMEGAAAAAAAAAAAAAAGAGGGGFPHPAAAAAGGNFSVAAAAAAAAAAAANQCRNLMAHPAPLAPGAASAYSSAPGEAPPSAAAAAAAAAAAAAAAAAASSSGGPGPAGPAGAEAAKQCSPCSAAAQSSSGPAALPYGYFGSGYYPCARMGPHPNAIKSCAQPASAAAAAAFADKYMDTAGPAAEEFSSRAKEFAFYHQGYAAGPYHHHQPMPGYLDMPVVPGLGGPGESRHEPLGLPMESYQPWALPNGWNGQMYCPKEQAQPPHLWKSTLPDVVSHPSDASSYRRGRKKRVPYTKVQLKELEREYATNKFITKDKRRRISATTNLSERQVTIWFQNRRVKEKKVINKLKTTS

SEQ ID NO: 108 - protein, 284 aa, synthetic construct
MEPGNYATLDGAKDIEGLLGAGGGRNLVAHSPLTSHPAAPTLMPAVNYAPLDLPGSAEPPKQCHPCPGVPQGTSPAPVPYGYFGGGYYSCRVSRSSLKPCAQAATLAAYPAETPTAGEEYPSRPTEFAFYPGYPGTYQPMASYLDVSVVQTLGAPGEPRHDSLLPVDSYQSWALAGGWNSQMCCQGEQNPPGPFWKAAFADSSGQHPPDACAFRRGRKKRIPYSKGQLRELEREYAANKFITKDKRRKISAATSLSERQITIWFQNRRVKEKKVLAKVKNSATP

SEQ ID NO: 109 - protein, 330 aa, synthetic construct
MTTSLLLHPRWPESLMYVYEDSAAESGIGGGGGGGGGGTGGAGGGCSGASPGKAPSMDGLGSSCPASHCRDLLPHPVLGRPPAPLGAPQGAVYTDIPAPEAARQCAPPPAPPTSSSATLGYGYPFGGSYYGCRLSHNVNLQQKPCAYHPGDKYPEPSGALPGDDLSSRAKEFAFYPSFASSYQAMPGYLDVSVVPGISGHPEPRHDALIPVEGYQHWALSNGWDSQVYCSKEQSQSAHLWKSPFPDVVPLQPEVSSYRRGRKKRVPYTKVQLKELEKEYAASKFITKEKRRRISATTNLSERQVTIWFQNRRVKEKKVVSKSKAPHLHST

SEQ ID NO: 110 - protein, 313 aa, synthetic construct
MDFDERGPCSSNMYLPSCTYYVSGPDFSSLPSFLPQTPSSRPMTYSYSSNLPQVQPVREVTFREYAIEPATKWHPRGNLAHCYSAEELVHRDCLQAPSAAGVPGDVLAKSSANVYHHPTPAVSSNFYSTVGRNGVLPQAFDQFFETAYGTPENLASSDYPGDKSAEKGPPAATATSAAAAAAATGAPATSSSDSGGGGGCRETAAAAEEKERRRRPESSSSPESSSGHTEDKAGGSSGQRTRKKRCPYTKYQIRELEREFFFSVYINKEKRLQLSRMLNLTDRQVKIWFQNRRMKEKKINRDRLQYYSANPLL

SEQ ID NO: 111 - protein, 304 aa, synthetic construct
MFNSVNLGNFCSPSRKERGADFGERGSCASNLYLPSCTYYMPEFSTVSSFLPQAPSRQISYPYSAQVPPVREVSYGLEPSGKWHHRNSYSSCYAAADELMHRECLPPSTVTEILMKNEGSYGGHHHPSAPHATPAGFYSSVNKNSVLPQAFDRFFDNAYCGGGDPPAEPPCSGKGEAKGEPEAPPASGLASRAEAGAEAEAEEENTNPSSSGSAHSVAKEPAKGAAPNAPRTRKKRCPYSKFQIRELEREFFFNVYINKEKRLQLSRMLNLTDRQVKIWFQNRRMKEKKLSRDRLQYFSGNPLL

SEQ ID NO: 112 - protein, 342 aa, synthetic construct
MTCPRNVTPNSYAEPLAAPGGGERYSRSAGMYMQSGSDFNCGVMRGCGLAPSLSKRDEGSSPSLALNTYPSYLSQLDSWGDPKAAYRLEQPVGRPLSSCSYPPSVKEENVCCMYSAEKRAKSGPEAALYSHPLPESCLGEHEVPVPSYYRASPSYSALDKTPHCSGANDFEAPFEQRASLNPRAEHLESPQLGGKVSFPETPKSDSQTPSPNEIKTEQSLAGPKGSPSESEKERAKAADSSPDTSDNEAKEEIKAENTTGNWLTAKSGRKKRCPYTKHQTLELEKEFLFNMYLTRERRLEISKTINLTDRQVKIWFQNRRMKLKKMNRENRIRELTSNFNFT

SEQ ID NO: 113 - protein, 410 aa, synthetic construct
MSARKGYLLPSPNYPTTMSCSESPAANSFLVDSLISSGRGEAGGGGGGAGGGGGGGYYAHGGVYLPPAADLPYGLQSCGLFPTLGGKRNEAASPGSGGGGGGLGPGAHGYGPSPIDLWLDAPRSCRMEPPDGPPPPPQQQPPPPPQPPQPAPQATSCSFAQNIKEESSYCLYDSADKCPKVSATAAELAPFPRGPPPDGCALGTSSGVPVPGYFRLSQAYGTAKGYGSGGGGAQQLGAGPFPAQPPGRGFDLPPALASGSADAARKERALDSPPPPTLACGSGGGSQGDEEAHASSSAAEELSPAPSESSKASPEKDSLGNSKGENAANWLTAKSGRKKRCPYTKHQTLELEKEFLFNMYLTRERRLEISRSVHLTDRQVKIWFQNRRMKLKKMNRENRIRELTANFNFS

SEQ ID NO: 114 - protein, 250 aa, synthetic construct
MSISGTLSSYYVDSIISHESEDAPPAKFPSGQYASSRQPGHAEHLEFPSCSFQPKAPVFGASWAPLSPHASGSLPSVYHPYIQPQGVPPAESRYLRTWLEPAPRGEAAPGQGQAAVKAEPLLGAPGELLKQGTPEYSLETSAGREAVLSNQRPGYGDNKICEGSEDKERPDQTNPSANWLHARSSRKKRCPYTKYQTLELEKEFLFNMYLTRDRRHEVARLLNLSERQVKIWFQNRRMKMKKMNKEQGKE

SEQ ID NO: 115 - protein, 272 aa, synthetic construct
MATTGALGNYYVDSFLLGADAADELSVGRYAPGTLGQPPRQAATLAEHPDFSPCSFQSKATVFGASWNPVHAAGANAVPAAVYHHHHHHPYVHPQAPVAAAAPDGRYMRSWLEPTPGALSFAGLPSSRPYGIKPEPLSARRGDCPTLDTHTLSLTDYACGSPPVDREKQPSEGAFSENNAENESGGDKPPIDPNNPAANWLHARSTRKKRCPYTKHQTLELEKEFLFNMYLTRDRRYEVARLLNLTERQVKIWFQNRRMKMKKINKDRAKDE

SEQ ID NO: 116 - protein, 80 aa, Homo sapiens
NKKLEAVGTGIEPKAMSQGLVTFGDVAVDFSQEEWEWLNPIQRNLYRKVMLENYRNLASLGLCVSKPDVISSLEQGKEPW

SEQ ID NO: 117 - protein, 80 aa, Homo sapiens
KMKKFQIPVSFQDLTVNFTQEEWQQLDPAQRLLYRDVMLENYSNLVSVGYHVSKPDVIFKLEQGEEPWIVEEFSNQNYPD

SEQ ID NO: 118 - protein, 80 aa, Homo sapiens
CSQESALSEEEEDTTRPLETVTFKDVAVDLTQEEWEQMKPAQRNLYRDVMLENYSNLVTVGCQVTKPDVIFKLEQEEEPW

SEQ ID NO: 119 - protein, 80 aa, Homo sapiens
ITSQGSVSFRDVTVGFTQEEWQHLDPAQRTLYRDVMLENYSHLVSVGYCIPKPEVILKLEKGEEPWILEEKFPSQSHLEL

SEQ ID NO: 120 - protein, 80 aa, Homo sapiens
PQVTFNDVAIDFTHEEWGWLSSAQRDLYKDVMVQNYENLVSVAGLSVTKPYVITLLEDGKEPWMMEKKLSKGMIPDWESR

SEQ ID NO: 121 - protein, 80 aa, Homo sapiens
PLRFSTLFQEQQKMNISQASVSFKDVTIEFTQEEWQQMAPVQKNLYRDVMLENYSNLVSVGYCCFKPEVIFKLEQGEEPW

SEQ ID NO: 122 - protein, 80 aa, Homo sapiens
QDPSAEGLSEEVPVVFEELPVVFEDVAVYFTREEWGMLDKRQKELYRDVMRMNYELLASLGPAAAKPDLISKLERRAAPW

SEQ ID NO: 123 - protein, 80 aa, Homo sapiens
SQGSVTFRDVAIDFSQEEWKWLQPAQRDLYRCVMLENYGHLVSLGLSISKPDVVSLLEQGKEPWLGKREVKRDLFSVSES

SEQ ID NO: 124 - protein, 80 aa, Homo sapiens
GSLTFRDVAIEFSLEEWQCLDTAQQNLYRNVMLENYRNLVFLGIAAFKPDLIIFLEEGKESWNMKRHEMVEESPVICSHF

SEQ ID NO: 125 - protein, 80 aa, Homo sapiens
NKSQEQVSFKDVCVDFTQEEWYLLDPAQKILYRDVILENYSNLVSVGYCITKPEVIFKIEQGEEPWILEKGFPSQCHPER

SEQ ID NO: 126 - protein, 80 aa, Homo sapiens
PHLLVTFRDVAIDFSQEEWECLDPAQRDLYRDVMLENYSNLISLDLESSCVTKKLSPEKEIYEMESLQWENMGKRINHHL

SEQ ID NO: 127 - protein, 80 aa, Homo sapiens
EEERKTAELQKNRIQDSVVFEDVAVDFTQEEWALLDLAQRNLYRDVMLENFQNLASLGYPLHTPHLISQWEQEEDLQTVK

SEQ ID NO: 128 - protein, 80 aa, Homo sapiens
GLLTFRDVAIEFSLEEWQCLDTAQKNLYRNVMLENYRNLAFLGIAVSKPDLIICLEKEKEPWNMKRDEMVDEPPGICPHF

SEQ ID NO: 129 - protein, 80 aa, Homo sapiens
RAPTQVTVSPETHMDLTKGCVTFEDIAIYFSQDEWGLLDEAQRLLYLEVMLENFALVASLGCGHGTEDEETPSDQNVSVG

SEQ ID NO: 130 - protein, 80 aa, Homo sapiens
AAVSPTTRCQESVTFEDVAVVFTDEEWSRLVPIQRDLYKEVMLENYNSIVSLGLPVPQPDVIFQLKRGDKPWMVDLHGSE

SEQ ID NO: 131 - protein, 80 aa, Homo sapiens
HSLEKVTFEDVAIDFTQEEWDMMDTSKRKLYRDVMLENISHLVSLGYQISKSYIILQLEQGKELWREGRVFLQDQNPDRE

SEQ ID NO: 132 - protein, 80 aa, Homo sapiens
DSVSFEDVAVNFTLEEWALLDSSQKKLYEDVMQETFKNLVCLGKKWEDQDIEDDHRNQGKNRRCHMVERLCESRRGSKCG

SEQ ID NO: 133 - protein, 80 aa, Homo sapiens
NVEVVKVMPQDLVTFKDVAIDFSQEEWQWMNPAQKRLYRSMMLENYQSLVSLGLCISKPYVISLLEQGREPWEMTSEMTR

SEQ ID NO: 134 - protein, 80 aa, Homo sapiens
TQPDEDLHLQAEETQLVKESVTFKDVAIDFTLEEWRLMDPTQRNLHKDVMLENYRNLVSLGLAVSKPDMISHLENGKGPW

SEQ ID NO: 135 - protein, 80 aa, Homo sapiens
TMLQESFSFDDLSVDFTQKEWQLLDPSQKNLYKDVMLENYSSLVSLGYEVMKPDVIFKLEQGEEPWVGDGEIPSSDSPEV

SEQ ID NO: 136 - protein, 80 aa, Homo sapiens
EVVTFGDVAVHFSREEWQCLDPGQRALYREVMLENHSSVAGLAGFLVFKPELISRLEQGEEPWVLDLQGAEGTEAPRTSK

SEQ ID NO: 137 - protein, 80 aa, Homo sapiens
RNAEEERMIAVFLTTWLQEPMTFKDVAVEFTQEEWMMLDSAQRSLYRDVMLENYRNLTSVEYQLYRLTVISPLDQEEIRN

SEQ ID NO: 138 - protein, 80 aa, Homo sapiens
GPQGARRQAFLAFGDVTVDFTQKEWRLLSPAQRALYREVTLENYSHLVSLGILHSKPELIRRLEQGEVPWGEERRRRPGP

SEQ ID NO: 139 - protein, 80 aa, Homo sapiens
HSLKKLTFEDVAIDFTQEEWAMMDTSKRKLYRDVMLENISHLVSLGYQISKSYIILQLEQGKELWREGRVFLQDQNPNRE

SEQ ID NO: 140 - protein, 80 aa, Homo sapiens
MPEVEFPDQFFTVLTMDHELVTLRDVVINFSQEEWEYLDSAQRNLYWDVMMENYSNLLSLDLESRNETKHLSVGKDIIQN

SEQ ID NO: 141 - protein, 80 aa, Homo sapiens
PGAPGSLEMGPLTFRDVTIEFSLEEWQCLDTVQQNLYRDVMLENYRNLVFLGMAVFKPDLITCLKQGKEPWNMKRHEMVT

SEQ ID NO: 142 - protein, 80 aa, Homo sapiens
RDPAQVPVAADLLTDHEEGYVTFEDVAVYFSQEEWRLLDDAQRLLYRNVMLENFTLLASLGLASSKTHEITQLESWEEPF

SEQ ID NO: 143 - protein, 80 aa, Homo sapiens
HSLKKVTFEDVAIDFTQEEWAMMDTSKRKLYRDVMLENISHLVSLGYQISKSYIILQLEQGKELWREGREFLQDQNPDRE

SEQ ID NO: 144 - protein, 80 aa, Homo sapiens
TKSKEAVTFKDVAVVFSEEELQLLDLAQRKLYRDVMLENFRNVVSVGHQSTPDGLPQLEREEKLWMMKMATQRDNSSGAK

SEQ ID NO: 145 - protein, 80 aa, Homo sapiens
SQVTFSDVAIDFSHEEWACLDSAQRDLYKDVMVQNYENLVSVGLSVTKPYVIMLLEDGKEPWMMEKKLSKAYPFPLSHSV

SEQ ID NO: 146 - protein, 80 aa, Homo sapiens
PGPLRSLEMESLQFRDVAVEFSLEEWHCLDTAQQNLYRDVMLENYRHLVFLGIIVSKPDLITCLEQGIKPLTMKRHEMIA

SEQ ID NO: 147 - protein, 80 aa, Homo sapiens
LQTTWPQESVTFEDVAVYFTQNQWASLDPAQRALYGEVMLENYANVASLVAFPFPKPALISHLERGEAPWGPDPWDTEIL

SEQ ID NO: 148 - protein, 80 aa, Homo sapiens
APLLAPRPGETRPGCRKPGTVSFADVAVYFSPEEWGCLRPAQRALYRDVMQETYGHLGALGFPGPKPALISWMEQESEAW

SEQ ID NO: 149 - protein, 80 aa, Homo sapiens
NKVEQKSQESVSFKDVTVGFTQEEWQHLDPSQRALYRDVMLENYSNLVSVGYCVHKPEVIFRLQQGEEPWKQEEEFPSQS

SEQ ID NO: 150 - protein, 80 aa, Homo sapiens
CFSQEERMAAGYLPRWSQELVTFEDVSMDFSQEEWELLEPAQKNLYREVMLENYRNVVSLEALKNQCTDVGIKEGPLSPA

SEQ ID NO: 151 - protein, 80 aa, Homo sapiens
DSVAFEDVAVNFTQEEWALLDPSQKNLYREVMQETLRNLTSIGKKWNNQYIEDEHQNPRRNLRRLIGERLSESKESHQHG

SEQ ID NO: 152 - protein, 80 aa, Homo sapiens
MGPAQVPMNSEVIVDPIQGQVNFEDVFVYFSQEEWVLLDEAQRLLYRDVMLENFALMASLGHTSFMSHIVASLVMGSEPW

SEQ ID NO: 153 - protein, 80 aa, Homo sapiens
TTFKEAMTFKDVAVVFTEEELGLLDLAQRKLYRDVMLENFRNLLSVGHQAFHRDTFHFLREEKIWMMKTAIQREGNSGDK

SEQ ID NO: 154 - protein, 80 aa, Homo sapiens
DSTLLQGGHNLLSSASFQEAVTFKDVIVDFTQEEWKQLDPGQRDLFRDVTLENYTHLVSIGLQVSKPDVISQLEQGTEPW

SEQ ID NO: 155 - protein, 80 aa, Homo sapiens
EARSMLVPPQASVCFEDVAMAFTQEEWEQLDLAQRTLYREVTLETWEHIVSLGLFLSKSDVISQLEQEEDLCRAEQEAPR

SEQ ID NO: 156 - protein, 80 aa, Homo sapiens
DSVVFEDVAVDFTLEEWALLDSAQRDLYRDVMLETFRNLASVDDGTQFKANGSVSLQDMYGQEKSKEQTIPNFTGNNSCA

SEQ ID NO: 157 - protein, 80 aa, Homo sapiens
EESHGALISSCNSRTMTDGLVTFRDVAIDFSQEEWECLDPAQRDLYVDVMLENYSNLVSLDLESKTYETKKIFSENDIFE

SEQ ID NO: 158 - protein, 80 aa, Homo sapiens
VITPQIPMVTEEFVKPSQGHVTFEDIAVYFSQEEWGLLDEAQRCLYHDVMLENFSLMASVGCLHGIEAEEAPSEQTLSAQ

SEQ ID NO: 159 - protein, 80 aa, Homo sapiens
VQLRPQTRMATALRDPASGSVTFEDVAVYFSWEEWDLLDEAQKHLYFDVMLENFALTSSLGCWCGVEHEETPSEQRISGE

SEQ ID NO: 160 - protein, 80 aa, Homo sapiens
MQAQESLTLEDVAVDFTWEEWQFLSPAQKDLYRDVMLENYSNLVAVGYQASKPDALSKLERGEETCTTEDEIYSRICSEI

SEQ ID NO: 161 - protein, 80 aa, Homo sapiens
GPLQFRDVAIEFSLEEWHCLDTAQRNLYRDVMLENYRNLVFLGIVVSKPDLVTCLEQGKKPLTMERHEMIAKPPVMSSHF

SEQ ID NO: 162 - protein, 80 aa, Homo sapiens
NMFKEAVTFKDVAVAFTEEELGLLGPAQRKLYRDVMVENFRNLLSVGHPPFKQDVSPIERNEQLWIMTTATRRQGNLGEK

SEQ ID NO: 163 - protein, 80 aa, Homo sapiens
GALTFRDVAIEFSLEEWQCLDTEQQNLYRNVMLDNYRNLVFLGIAVSKPDLITCLEQEKEPWNLKTHDMVAKPPVICSHI

SEQ ID NO: 164 - protein, 80 aa, Homo sapiens
SPQKSSALAPEDHGSSYEGSVSFRDVAIDFSREEWRHLDPSQRNLYRDVMLETYSHLLSVGYQVPEAEVVMLEQGKEPWA

SEQ ID NO: 165 - protein, 80 aa, Homo sapiens
ELLTFRDVAIEFSPEEWKCLDPAQQNLYRDVMLENYRNLISLGVAISNPDLVIYLEQRKEPYKVKIHETVAKHPAVCSHF

SEQ ID NO: 166 - protein, 80 aa, Homo sapiens
EPLKFRDVAIEFSLEEWQCLDTIQQNLYRNVMLENYRNLVFLGIVVSKPDLITCLEQEKEPWTRKRHRMVAEPPVICSHF

SEQ ID NO: 167 - protein, 80 aa, Homo sapiens
PSARGKSKSKAPITFGDLAIYFSQEEWEWLSPIQKDLYEDVMLENYRNLVSLGLSFRRPNVITLLEKGKAPWMVEPVRRR

SEQ ID NO: 168 - protein, 80 aa, Homo sapiens
TKAQESLTLEDVAVDFTWEEWQFLSPAQKDLYRDVMLENYSNLVSVGYQAGKPDALTKLEQGEPLWTLEDEIHSPAHPEI

SEQ ID NO: 169 - protein, 80 aa, Homo sapiens
SQEEVEVAGIKLCKAMSLGSVTFTDVAIDFSQDEWEWLNLAQRSLYKKVMLENYRNLVSVGLCISKPDVISLLEQEKDPW

SEQ ID NO: 170 - protein, 80 aa, Homo sapiens
TKSLESVSFKDVTVDFSRDEWQQLDLAQKSLYREVMLENYFNLISVGCQVPKPEVIFSLEQEEPCMLDGEIPSQSRPDGD

SEQ ID NO: 171 - protein, 80 aa, Homo sapiens
SGCPGAERNLLVYSYFEKETLTFRDVAIEFSLEEWECLNPAQQNLYMNVMLENYKNLVFLGVAVSKQDPVTCLEQEKEPW

SEQ ID NO: 172 - protein, 80 aa, Homo sapiens
PLQGSVSFKDVTVDFTQEEWQQLDPAQKALYRDVMLENYCHFVSVGFHMAKPDMIRKLEQGEELWTQRIFPSYSYLEEDG

SEQ ID NO: 173 - protein, 80 aa, Homo sapiens
PGPPRSLEMGLLTFRDVAIEFSLEEWQHLDIAQQNLYRNVMLENYRNLAFLGIAVSKPDLITCLEQGKEPWNMKRHEMVD

SEQ ID NO: 174 - protein, 80 aa, Homo sapiens
SGHPGSWEMNSVAFEDVAVNFTQEEWALLDPSQKNLYRDVMQETFRNLASIGNKGEDQSIEDQYKNSSRNLRHIISHSGN

SEQ ID NO: 175 - protein, 80 aa, Homo sapiens
SYGSITFGDVAIDFSHQEWEYLSLVQKTLYQEVMMENYDNLVSLAGHSVSKPDLITLLEQGKEPWMIVREETRGECTDLD

SEQ ID NO: 176 - protein, 80 aa, Homo sapiens
DLFVCSGLEPHTPSVGSQESVTFQDVAVDFTEKEWPLLDSSQRKLYKDVMLENYSNLTSLGYQVGKPSLISHLEQEEEPR

SEQ ID NO: 177 - protein, 80 aa, Homo sapiens
FQTAWRQEPVTFEDVAVYFTQNEWASLDSVQRALYREVMLENYANVASLAFPFTTPVLVSQLEQGELPWGLDPWEPMGRE

SEQ ID NO: 178 - protein, 80 aa, Homo sapiens
ELLTFRDVAIEFSPEEWKCLDPDQQNLYRDVMLENYRNLVSLGVAISNPDLVTCLEQRKEPYNVKIHKIVARPPAMCSHF

SEQ ID NO: 179 - protein, 80 aa, Homo sapiens
AGEAEAQLDPSLQGLVMFEDVTVYFSREEWGLLNVTQKGLYRDVMLENFALVSSLGLAPSRSPVFTQLEDDEQSWVPSWV

SEQ ID NO: 180 - protein, 80 aa, Homo sapiens
AHALVTFRDVAIDFSQKEWECLDTTQRKLYRDVMLENYNNLVSLGYSGSKPDVITLLEQGKEPCVVARDVTGRQCPGLLS

SEQ ID NO: 181 - protein, 80 aa, Homo sapiens
KRIKHWKMASKLILPESLSLLTFEDVAVYFSEEEWQLLNPLEKTLYNDVMQDIYETVISLGLKLKNDTGNDHPISVSTSE

SEQ ID NO: 182 - protein, 80 aa, Homo sapiens
DSVVFEDVAVDFTLEEWALLDSAQRDLYRDVMLETFQNLASVDDETQFKASGSVSQQDIYGEKIPKESKIATFTRNVSWA

SEQ ID NO: 183 - protein, 80 aa, Homo sapiens
NMSQASVSFQDVTVEFTREEWQHLGPVERTLYRDVMLENYSHLISVGYCITKPKVISKLEKGEEPWSLEDEFLNQRYPGY

SEQ ID NO: 184 - protein, 80 aa, Homo sapiens
ISFQESVTFQDVAVDFTAEEWQLLDCAERTLYWDVMLENYRNLISVGCPITKTKVILKVEQGQEPWMVEGANPHESSPES

SEQ ID NO: 185 - protein, 80 aa, Homo sapiens
NTLQGPVSFKDVAVDFTQEEWQQLDPDEKITYRDVMLENYSHLVSVGYDTTKPNVIIKLEQGEEPWIMGGEFPCQHSPEA

SEQ ID NO: 186 - protein, 80 aa, Homo sapiens
HPEEEERMHDELLQAVSKGPVMFRDVSIDFSQEEWECLDADQMNLYKEVMLENFSNLVSVGLSNSKPAVISLLEQGKEPW

SEQ ID NO: 187 - protein, 80 aa, Homo sapiens
SLGSELFRDVAIVFSQEEWQWLAPAQRDLYRDVMLETYSNLVSLGLAVSKPDVISFLEQGKEPWMVERVVSGGLCPVLES

SEQ ID NO: 188 - protein, 80 aa, Homo sapiens
TKFQEMVTFKDVAVVFTEEELGLLDSVQRKLYRDVMLENFRNLLLVAHQPFKPDLISQLEREEKLLMVETETPRDGCSGR

SEQ ID NO: 189 - protein, 80 aa, Homo sapiens
AKRPGPPGSREMGLLTFRDIAIEFSLAEWQCLDHAQQNLYRDVMLENYRNLVSLGIAVSKPDLITCLEQNKEPQNIKRNE

SEQ ID NO: 190 - protein, 80 aa, Homo sapiens
TCMVHRQTMSCSGAGGITAFVAFRDVAVYFTQEEWRLLSPAQRTLHREVMLETYNHLVSLEIPSSKPKLIAQLERGEAPW

SEQ ID NO: 191 - protein, 80 aa, Homo sapiens
IQAQESITLEDVAVDFTWEEWQLLGAAQKDLYRDVMLENYSNLVAVGYQASKPDALFKLEQGEQLWTIEDGIHSGACSDI

SEQ ID NO: 192 - protein, 80 aa, Homo sapiens
AQKRRKRKAKESGMALPQGHLTFRDVAIEFSQAEWKCLDPAQRALYKDVMLENYRNLVSLGISLPDLNINSMLEQRREPW

SEQ ID NO: 193 - protein, 80 aa, Homo sapiens
DSTSVPVTAEAKLMGFTQGCVTFEDVAIYFSQEEWGLLDEAQRLLYRDVMLENFALITALVCWHGMEDEETPEQSVSVEG

SEQ ID NO: 194 - protein, 80 aa, Homo sapiens
GPLTFRDVKIEFSLEEWQCLDTAQRNLYRDVMLENYRNLVFLGIAVSKPDLITWLEQGKEPWNLKRHEMVDKTPVMCSHF

SEQ ID NO: 195 - protein, 80 aa, Homo sapiens
SGCPGAERSLLVQSYFEKGPLTFRDVAIEFSLEEWQCLDSAQQGLYRKVMLENYRNLVFLAGIALTKPDLITCLEQGKEP

SEQ ID NO: 196 - protein, 80 aa, Homo sapiens
GVLTFRDVAVEFSPEEWECLDSAQQRLYRDVMLENYGNLVSLGLAIFKPDLMTCLEQRKEPWKVKRQEAVAKHPAGSFHF

SEQ ID NO: 197 - protein, 80 aa, Homo sapiens
KENLEDISGWGLPEARSKESVSFKDVAVDFTQEEWGQLDSPQRALYRDVMLENYQNLLALGPPLHKPDVISHLERGEEPW

SEQ ID NO: 198 - protein, 80 aa, Homo sapiens
EESEKPRGFARGLEPERIIGATDSSGELMFLMKWKNSDEADLVPAKEANVKCPQVVISFYEERLTWHSYPSEDDDKKDDK

SEQ ID NO: 199 - protein, 80 aa, Homo sapiens
ASVALEDVAVNFTREEWALLGPCQKNLYKDVMQETIRNLDCVVMKWKDQNIEDQYRYPRKNLRCRMLERFVESKDGTQCG

SEQ ID NO: 200 - protein, 80 aa, Homo sapiens
TLLTFRDVAIEFSLEEWKCLDLAQQNLYRDVMLENYRNLFSVGLTVCKPGLITCLEQRKEPWNVKRQEAADGHPEMGFHH

SEQ ID NO: 201 - protein, 80 aa, Homo sapiens
AAALRAPTQQVFVAFEDVAIYFSQEEWELLDEMQRLLYRDVMLENFAVMASLGCWCGAVDEGTPSAESVSVEELSQGRTP

SEQ ID NO: 202 - protein, 80 aa, Homo sapiens
NTFQASVSFQDVTVEFSQEEWQHMGPVERTLYRDVMLENYSHLVSVGYCFTKPELIFTLEQGEDPWLLEKEKGFLSRNSP

SEQ ID NO: 203 - protein, 80 aa, Homo sapiens
DSVAFEDVSVSFSQEEWALLAPSQKKLYRDVMQETFKNLASIGEKWEDPNVEDQHKNQGRNLRSHTGERLCEGKEGSQCA

SEQ ID NO: 204 - protein, 80 aa, Homo sapiens
AQGLVTFADVAIDFSQEEWACLNSAQRDLYWDVMLENYSNLVSLDLESAYENKSLPTEKNIHEIRASKRNSDRRSKSLGR

SEQ ID NO: 205 - protein, 80 aa, Homo sapiens
AVDLLSAQEPVTFRDVAVFFSQDEWLHLDSAQRALYREVMLENYSSLVSLGIPFSMPKLIHQLQQGEDPCMVEREVPSDT

SEQ ID NO: 206 - protein, 80 aa, Homo sapiens
SPQRFPALIPGEPGRSFEGSVSFEDVAVDFTRQEWHRLDPAQRTMHKDVMLETYSNLASVGLCVAKPEMIFKLERGEELW

SEQ ID NO: 207 - protein, 80 aa, Homo sapiens
RVLTFRDVAVEFSPEEWECLDSAQQRLYRDVMLENYGNLFSLGLAIFKPDLITYLEQRKEPWNARRQKTVAKHPAGSLHF

SEQ ID NO: 208 - protein, 80 aa, Homo sapiens
AETKDAAQMLVTFKDVAVTFTREEWRQLDLAQRTLYREVMLETCGLLVSLGHRVPKPELVHLLEHGQELWIVKRGLSHAT

SEQ ID NO: 209 - protein, 80 aa, Homo sapiens
IEYQIPVSFKDVVVGFTQEEWHRLSPAQRALYRDVMLETYSNLVSVGYEGTKPDVILRLEQEEAPWIGEAACPGCHCWED

SEQ ID NO: 210 - protein, 80 aa, Homo sapiens
TKFQEAVTFKDVAVAFTEEELGLLDSAQRKLYRDVMLENFRNLVSVGHQSFKPDMISQLEREEKLWMKELQTQRGKHSGD

SEQ ID NO: 211 - protein, 80 aa, Homo sapiens
DEGVAGVMSVGPPAARLQEPVTFRDVAVDFTQEEWGQLDPTQRILYRDVMLETFGHLLSIGPELPKPEVISQLEQGTELW

SEQ ID NO: 212 - protein, 80 aa, Homo sapiens
GPLTFMDVAIEFSVEEWQCLDTAQQNLYRNVMLENYRNLVFLGIAVSKPDLITCLEQGKEPWNMERHEMVAKPPGMCCYF

SEQ ID NO: 213 - protein, 80 aa, Homo sapiens
HQVGLIRSYNSKTMTCFQELVTFRDVAIDFSRQEWEYLDPNQRDLYRDVMLENYRNLVSLGGHSISKPVVVDLLERGKEP

SEQ ID NO: 214 - protein, 80 aa, Homo sapiens
NATIVMSVRREQGSSSGEGSLSFEDVAVGFTREEWQFLDQSQKVLYKEVMLENYINLVSIGYRGTKPDSLFKLEQGEPPG

SEQ ID NO: 215 - protein, 80 aa, Homo sapiens
FPPARGKELLSFEDVAMYFTREEWGHLNWGQKDLYRDVMLENYRNMVLLGFQFPKPEMICQLENWDEQWILDLPRTGNRK

SEQ ID NO: 216 - protein, 80 aa, Homo sapiens
ELLTFKDVAIEFSPEEWKCLDTSQQNLYRDVMLENYRNLVSLGVSISNPDLVTSLEQRKEPYNLKIHETAARPPAVCSHF

SEQ ID NO: 217 - protein, 80 aa, Homo sapiens
MKSQGLVSFKDVAVDFTQEEWQQLDPSQRTLYRDVMLENYSHLVSMGYPVSKPDVISKLEQGEEPWIIKGDISNWIYPDE

SEQ ID NO: 218 - protein, 80 aa, Homo sapiens
AEGSVMFSDVSIDFSQEEWDCLDPVQRDLYRDVMLENYGNLVSMGLYTPKPQVISLLEQGKEPWMVGRELTRGLCSDLES

SEQ ID NO: 219 - protein, 80 aa, Homo sapiens
GPLTFTDVAIEFSLEEWQCLDTAQQNLYRNVMLENYRNLVFLGIAVSKPDLITCLEKEKEPCKMKRHEMVDEPPVVCSHF

SEQ ID NO: 220 - protein, 80 aa, Homo sapiens
ALSQGLFTFKDVAIEFSQEEWECLDPAQRALYRDVMLENYRNLLSLDEDNIPPEDDISVGFTSKGLSPKENNKEELYHLV

SEQ ID NO: 221 - protein, 80 aa, Homo sapiens
NMEGLVMFQDLSIDFSQEEWECLDAAQKDLYRDVMMENYSSLVSLGLSIPKPDVISLLEQGKEPWMVSRDVLGGWCRDSE

SEQ ID NO: 222 - protein, 80 aa, Homo sapiens
AVSHLPTMVQESVTFKDVAILFTQEEWGQLSPAQRALYRDVMLENYSNLVSLGLLGPKPDTFSQLEKREVWMPEDTPGGF

SEQ ID NO: 223 - protein, 80 aa, Homo sapiens
GPLTIRDVTVEFSLEEWHCLDTAQQNLYRDVMLENYRNLVFLGIAVSKPDLITCLEQGKEPCNMKRHEMVAKPPVMCSHI

SEQ ID NO: 224 - protein, 80 aa, Homo sapiens
AAAVLTDRAQVSVTFDDVAVTFTKEEWGQLDLAQRTLYQEVMLENCGLLVSLGCPVPKAELICHLEHGQEPWTRKEDLSQ

SEQ ID NO: 225 - protein, 80 aa, Homo sapiens
ALRSVMFSDVSIDFSPEEWEYLDLEQKDLYRDVMLENYSNLVSLGCFISKPDVISSLEQGKEPWKVVRKGRRQYPDLETK

SEQ ID NO: 226 - protein, 80 aa, Homo sapiens
AHGSVTFRDVAIDFSQEEWEFLDPAQRDLYRDVMWENYSNFISLGPSISKPDVITLLDEERKEPGMVVREGTRRYCPDLE

SEQ ID NO: 227 - protein, 80 aa, Homo sapiens
APRAQIQGPLTFGDVAVAFTRIEWRHLDAAQRALYRDVMLENYGNLVSVGLLSSKPKLITQLEQGAEPWTEVREAPSGTH

SEQ ID NO: 228 - protein, 80 aa, Homo sapiens
GPLQFRDVAIEFSLEEWHCLDTAQRNLYRNVMLENYRNLVFLGIVVSKPDLITCLEQGKKPLTMKKHEMVANPSVTCSHF

SEQ ID NO: 229 - protein, 80 aa, Homo sapiens
TLPRGQPEVLEWGLPKDQDSVAFEDVAVNFTHEEWALLGPSQKNLYRDVMRETIRNLNCIGMKWENQNIDDQHQNLRRNP

SEQ ID NO: 230 - protein, 80 aa, Homo sapiens
PSPDSMTFEDIIVDFTQEEWALLDTSQRKLFQDVMLENISHLVSIGKQLCKSVVLSQLEQVEKLSTQRISLLQGREVGIK

SEQ ID NO: 231 - protein, 80 aa, Homo sapiens
EESREIRAGQIVLKAMAQGLVTFRDVAIEFSLEEWKCLEPAQRDLYREVTLENFGHLASLGLSISKPDVVSLLEQGKEPW

SEQ ID NO: 232 - protein, 80 aa, Homo sapiens
AASAQVSVTFEDVAVTFTQEEWGQLDAAQRTLYQEVMLETCGLLMSLGCPLFKPELIYQLDHRQELWMATKDLSQSSYPG

SEQ ID NO: 233 - protein, 80 aa, Homo sapiens
RESLEDEVTPGLPTAESQELLTFKDISIDFTQEEWGQLAPAHQNLYREVMLENYSNLVSVGYQLSKPSVISQLEKGEEPW

SEQ ID NO: 234 - protein, 80 aa, Homo sapiens
EGEYIKLKVIGQDSSEIHFKVKMTTHLKKLKESYCQRQGVPMNSLRFLFEGQRIADNHTPKELGMEEEDVIEVYQEQTGG

SEQ ID NO: 235 - protein, 80 aa, Homo sapiens
NKSLGPVSFKDVAVDFTQEEWQQLDPEQKITYRDVMLENYSNLVSVGYHIIKPDVISKLEQGEEPWIVEGEFLLQSYPDE

SEQ ID NO: 236 - protein, 80 aa, Homo sapiens
SPGLLTTRKEALMAFRDVAVAFTQKEWKLLSSAQRTLYREVMLENYSHLVSLGIAFSKPKLIEQLEQGDEPWREENEHLL

SEQ ID NO: 237 - protein, 80 aa, Homo sapiens
MFQDSVAFEDVAVTFTQEEWALLDPSQKNLCRDVMQETFRNLASIGKKWKPQNIYVEYENLRRNLRIVGERLFESKEGHQ

SEQ ID NO: 238 - protein, 80 aa, Homo sapiens
ENDHINLKVAGQDGSVVQFKIKRHTPLSKLMKAYCERQGLSMRQIRFRFDGQPINETDTPAQLEMEDEDTIDVFQQQTGG

SEQ ID NO: 239 - protein, 80 aa, Homo sapiens
PGPLGSLEMGVLTFRDVALEFSLEEWQCLDTAQQNLYRNVMLENYRNLVFVGIAASKPDLITCLEQGKEPWNVKRHEMVT

SEQ ID NO: 240 - protein, 80 aa, Homo sapiens
LSQKPQVLGPEKQDGSCEASVSFEDVTVDFSREEWQQLDPAQRCLYRDVMLELYSHLFAVGYHIPNPEVIFRMLKEKEPR

SEQ ID NO: 241 - protein, 80 aa, Homo sapiens
ALTQGQVTFRDVAIEFSQEEWTCLDPAQRTLYRDVMLENYRNLASLGISCFDLSIISMLEQGKEPFTLESQVQIAGNPDG

SEQ ID NO: 242 - protein, 80 aa, Homo sapiens
NKFQGPVTLKDVIVEFTKEEWKLLTPAQRTLYKDVMLENYSHLVSVGYHVNKPNAVFKLKQGKEPWILEVEFPHRGFPED

SEQ ID NO: 243 - protein, 80 aa, Homo sapiens
ELLTFRDVAIEFSPEEWKCLDPAQQNLYRDVMLENYRNLVSLAVYSYYNQGILPEQGIQDSFKKATLGRYGSCGLENICL

SEQ ID NO: 244 - protein, 80 aa, Homo sapiens
SPQKSSALAPEDHGSSYEGSVSFRDVAIDFSREEWRHLDLSQRNLYRDVMLETYSHLLSVGYQVPKPEVVMLEQGKEPWA

SEQ ID NO: 245 - protein, 80 aa, Homo sapiens
NNSQGRVTFEDVTVNFTQGEWQRLNPEQRNLYRDVMLENYSNLVSVGQGETTKPDVILRLEQGKEPWLEEEEVLGSGRAE

SEQ ID NO: 246 - protein, 80 aa, Homo sapiens
AMALPMPGPQEAVVFEDVAVYFTRIEWSCLAPDQQALYRDVMLENYGNLASLGFLVAKPALISLLEQGEEPGALILQVAE

SEQ ID NO: 247 - protein, 80 aa, Homo sapiens
DSSQHLVTFEDVAVDFTQEEWTLLDQAQRDLYRDVMLENYKNLIILAGSELFKRSLMSGLEQMEELRTGVTGVLQELDLQ

SEQ ID NO: 248 - protein, 80 aa, Homo sapiens
TTFKEAVTFKDVAVFFTEEELGLLDPAQRKLYQDVMLENFTNLLSVGHQPFHPFHFLREEKFWMMETATQREGNSGGKTI

SEQ ID NO: 249 - protein, 80 aa, Homo sapiens
GPLQFRDVAIEFSLEEWHCLDMAQRNLYRDVMLENYRNLVFLGIVVSKPDLITHLEQGKKPSTMQRHEMVANPSVLCSHF

SEQ ID NO: 250 - protein, 80 aa, Homo sapiens
NKSQGSVSFTDVTVDFTQEEWEQLDPSQRILYMDVMLENYSNLLSVEVWKADDQMERDHRNPDEQARQFLILKNQTPIEE

SEQ ID NO: 251 - protein, 80 aa, Homo sapiens
EEEEMNDGSQMVRSQESLTFQDVAVDFTREEWDQLYPAQKNLYRDVMLENYRNLVALGYQLCKPEVIAQLELEEEWVIER

SEQ ID NO: 252 - protein, 80 aa, Homo sapiens
EEATKKSKEKEPGMALPQGRLTFRDVAIEFSLEEWKCLNPAQRALYRAVMLENYRNLEFVDSSLKSMMEFSSTRHSITGE

SEQ ID NO: 253 - protein, 80 aa, Homo sapiens
EKTPAGRIVADCLTDCYQDSVTFDDVAVDFTQEEWTLLDSTQRSLYSDVMLENYKNLATVGGQIIKPSLISWLEQEESRT

SEQ ID NO: 254 - protein, 80 aa, Homo sapiens
AMSQESLTFKDVFVDFTLEEWQQLDSAQKNLYRDVMLENYSHLVSVGHLVGKPDVIFRLGPGDESWMADGGTPVRTCAGE

SEQ ID NO: 255 - protein, 80 aa, Homo sapiens
DSVAFEDVAVNFTLEEWALLDPSQKNLYRDVMRETFRNLASVGKQWEDQNIEDPFKIPRRNISHIPERLCESKEGGQGEE

SEQ ID NO: 256 - protein, 80 aa, Homo sapiens
MFQDSVAFEDVAVSFTQEEWALLDPSQKNLYRDVMQETFKNLTSVGKTWKVQNIEDEYKNPRRNLSLMREKLCESKESHH

SEQ ID NO: 257 - protein, 80 aa, Homo sapiens
AVVATLRLSAQGTVTFEDVAVKFTQEEWNLLSEAQRCLYRDVTLENLALMSSLGCWCGVEDEAAPSKQSIYIQRETQVRT

SEQ ID NO: 258 - protein, 80 aa, Homo sapiens
EEEEEDEDEDDLLTAGCQELVTFEDVAVYFSLEEWERLEADQRGLYQEVMQENYGILVSLGYPIPKPDLIFRLEQGEEPW

SEQ ID NO: 259 - protein, 80 aa, Homo sapiens
TKFQEMVTFKDVAVVFTREELGLLDLAQRKLYQDVMLENFRNLLSVGYQPFKLDVILQLGKEDKLRMMETEIQGDGCSGH

SEQ ID NO: 260 - protein, 80 aa, Homo sapiens
EEAAQKRKGKEPGMALPQGRLTFRDVAIEFSLAEWKCLNPSQRALYREVMLENYRNLEAVDISSKCMMKEVLSTGQGNTE

SEQ ID NO: 261 - protein, 80 aa, Homo sapiens
DTVVFEDVVVDFTLEEWALLNPAQRKLYRDVMLETFKHLASVDNEAQLKASGSISQQDTSGEKLSLKQKIEKFTRKNIWA

SEQ ID NO: 262 - protein, 80 aa, Homo sapiens
TTFKEGLTFKDVAVVFTEEELGLLDPVQRNLYQDVMLENFRNLLSVGHHPFKHDVFLLEKEKKLDIMKTATQRKGKSADK

SEQ ID NO: 263 - protein, 80 aa, Homo sapiens
SALQQEFWKIQTSNGIQMDLVTFDSVAVEFTQEEWTLLDPAQRNLYSDVMLENYKNLSSVGYQLFKPSLISWLEEEEELS

SEQ ID NO: 264 - protein, 80 aa, Homo sapiens
DCVIFEEVAVNFTPEEWALLDHAQRSLYRDVMLETCRNLASLDCYIYVRTSGSSSQRDVFGNGISNDEEIVKFTGSDSWS

SEQ ID NO: 265 - protein, 80 aa, Homo sapiens
ELLTFRDVTIEFSLEEWEFLNPAQQSLYRKVMLENYRNLVSLGLTVSKPELISRLEQRQEPWNVKRHETIAKPPAMSSHY

SEQ ID NO: 266 - protein, 80 aa, Homo sapiens
IKTQESLTLEDVAVEFSWEEWQLLDTAQKNLYRDVMVENYNHLVSLGYQTSKPDVLSKLAHGQEPWTTDAKIQNKNCPGI

SEQ ID NO: 267 - protein, 80 aa, Homo sapiens
PAHVPGEAGPRRTRESRPGAVSFADVAVYFSPEEWECLRPAQRALYRDVMRETFGHLGALGFSVPKPAFISWVEGEVEAW

SEQ ID NO: 268 - protein, 80 aa, Homo sapiens
GCPGDQVTPTRSLTAQLQETMTFKDVEVTFSQDEWGWLDSAQRNLYRDVMLENYRNMASLVGPFTKPALISWLEAREPWG

SEQ ID NO: 269 - protein, 80 aa, Homo sapiens
ARDLVMFRDVAVDFSQEEWECLNSYQRNLYRDVILENYSNLVSLAGCSISKPDVITLLEQGKEPWMVVRDEKRRWTLDLE

SEQ ID NO: 270 - protein, 80 aa, Homo sapiens
TTLKEAVTFKDVAVVFTEEELRLLDLAQRKLYREVMLENFRNLLSVGHQSLHRDTFHFLKEEKFWMMETATQREGNLGGK

SEQ ID NO: 271 - protein, 80 aa, Homo sapiens
SPPSPRSSMAAVALRDSAQGMTFEDVAIYFSQEEWELLDESQRFLYCDVMLENFAHVTSLGYCHGMENEAIASEQSVSIQ

SEQ ID NO: 272 - protein, 80 aa, Homo sapiens
DEEAQKRKAKESGMALPQGRLTFMDVAIEFSQEEWKSLDPGQRALYRDVMLENYRNLVFLGICLPDLSIISMLKQRREPL

SEQ ID NO: 273 - protein, 80 aa, Homo sapiens
ALTQGPLKFMDVAIEFSQEEWKCLDPAQRTLYRDVMLENYRNLVSLGICLPDLSVTSMLEQKRDPWTLQSEEKIANDPDG

SEQ ID NO: 274 - protein, 80 aa, Homo sapiens
TMFKEAVTFKDVAVVFTEEELGLLDVSQRKLYRDVMLENFRNLLSVGHQLSHRDTFHFQREEKFWIMETATQREGNSGGK

SEQ ID NO: 275 - protein, 80 aa, Homo sapiens
QGTVAFEDVAVNFSQEEWSLLSEVQRCLYHDVMLENWVLISSLGCWCGSEDEEAPSKKSISIQRVSQVSTPGAGVSPKKA

SEQ ID NO: 276 - protein, 80 aa, Homo sapiens
AEAFGDSEEDGEDVFEVEKILDMKTEGGKVLYKVRWKGYTSDDDTWEPEIHLEDCKEVLLEFRKKIAENKAKAVRKDIQR

SEQ ID NO: 277 - protein, 80 aa, Homo sapiens
VLQMQNSEHHGQSIKTQTDSISLEDVAVNFTLEEWALLDPGQRNIYRDVMRATFKNLACIGEKWKDQDIEDEHKNQGRNL

SEQ ID NO: 278 - protein, 80 aa, Homo sapiens
AMALTDPAQVSVTFDDVAVTFTQEEWGQLDLAQRTLYQEVMLENCGLLVSLGCPVPRPELIYHLEHGQEPWTRKEDLSQG

SEQ ID NO: 279 - protein, 80 aa, Homo sapiens
VHGSVTFRDVAIDFSQEEWECLQPDQRTLYRDVMLENYSHLISLGSSISKPDVITLLEQEKEPWIVVSKETSRWYPDLES

SEQ ID NO: 280 - protein, 80 aa, Homo sapiens
DPVACEDVAVNFTQEEWALLDISQRKLYREVMLETFRNLTSIGKKWKDQNIEYEYQNPRRNFRSLIEGNVNEIKEDSHCG

SEQ ID NO: 281 - protein, 80 aa, Homo sapiens
IKFQERVTFKDVAVVFTKEELALLDKAQINLYQDVMLENFRNLMLVRDGIKNNILNLQAKGLSYLSQEVLHCWQIWKQRI

SEQ ID NO: 282 - protein, 80 aa, Homo sapiens
GPLTFRDVAIEFSLKEWQCLDTAQRNLYRNVMLENYRNLVFLGITVSKPDLITCLEQGKEAWSMKRHEIMVAKPTVMCSH

SEQ ID NO: 283 - protein, 80 aa, Homo sapiens
TMSKEAVTFKDVAVVFTEEELGLLDLAQRKLYRDVMLENFRNLLSVGHQPFHRDTFHFLREEKFWMMDIATQREGNSGGK

SEQ ID NO: 284 - protein, 80 aa, Homo sapiens
GPLEFRDVAIEFSLEEWHCLDTAQQNLYRDVMLENYRHLVFLGIVVTKPDLITCLEQGKKPFTVKRHEMIAKSPVMCFHF

SEQ ID NO: 285 - protein, 80 aa, Homo sapiens
GHTEGGELVNELLKSWLKGLVTFEDVAVEFTQEEWALLDPAQRTLYRDVMLENCRNLASLGNQVDKPRLISQLEQEDKVM

SEQ ID NO: 286 - protein, 80 aa, Homo sapiens
AEPASVTVTFDDVALYFSEQEWEILEKWQKQMYKQEMKTNYETLDSLGYAFSKPDLITWMEQGRMLLISEQGCLDKTRRT

SEQ ID NO: 287 - protein, 80 aa, Homo sapiens
HSQASAISQDREEKIMSQEPLSFKDVAVVFTEEELELLDSTQRQLYQDVMQENFRNLLSVGERNPLGDKNGKDTEYIQDE

SEQ ID NO: 288 - protein, 80 aa, Homo sapiens
GSLEEGRRATGLPAAQVQEPVTFKDVAVDFTQEEWGQLDLVQRTLYRDVMLETYGHLLSVGNQIAKPEVISLLEQGEEPW

SEQ ID NO: 289 - protein, 80 aa, Homo sapiens
TTFKEAVTFKDVAVVFTEEELGLLDPAQRKLYRDVMLENFRNLLSVGHQPFHQDTCHFLREEKFWMMGTATQREGNSGGK

SEQ ID NO: 290 - protein, 80 aa, Homo sapiens
AKLYEAVTFKDVAVIFTEEELGLLDPAQRKLYRDVMLENFRNLLSVGGKIQTEMETVPEAGTHEEFSCKQIWEQIASDLT

SEQ ID NO: 291 - protein, 80 aa, Homo sapiens
RSDLFLPDSQTNEERKQYDSVAFEDVAVNFTQEEWALLGPSQKSLYRDVMWETIRNLDCIGMKWEDTNIEDQHRNPRRSL

SEQ ID NO: 292 - protein, 80 aa, Homo sapiens
PGTPGSLEMGLLTFRDVAIEFSPEEWQCLDTAQQNLYRNVMLENYRNLAFLGIALSKPDLITYLEQGKEPWNMKQHEMVD

SEQ ID NO: 293 - protein, 80 aa, Homo sapiens
TPGVRVSTDPEQVTFEDVVVGFSQEEWGQLKPAQRTLYRDVMLDTFRLLVSVGHWLPKPNVISLLEQEAELWAVESRLPQ

SEQ ID NO: 294 - protein, 80 aa, Homo sapiens
EQTQAAGMVAGWLINCYQDAVTFDDVAVDFTQEEWTLLDPSQRDLYRDVMLENYENLASVEWRLKTKGPALRQDRSWFRA

SEQ ID NO: 295 - protein, 80 aa, Homo sapiens
PSEANSIQSANATTKTSETNHTSRPRLKNVDRSTAQQLAVTVGNVTVIITDFKEKTRSSSTSSSTVTSSAGSEQQNQSSS

SEQ ID NO: 296 - protein, 80 aa, Homo sapiens
ALTQGQLSFSDVAIEFSQEEWKCLDPGQKALYRDVMLENYRNLVSLGEDNVRPEACICSGICLPDLSVTSMLEQKRDPWT

SEQ ID NO: 297 - protein, 80 aa, Homo sapiens
AAAAALRAPAQSSVTFEDVAVNFSLEEWSLLNEAQRCLYRDVMLETLTLISSLGCWHGGEDEAAPSKQSTCIHIYKDQGG

SEQ ID NO: 298 - protein, 80 aa, Homo sapiens
AQGSVSFNDVTVDFTQEEWQHLDHAQKTLYMDVMLENYCHLISVGCHMTKPDVILKLERGEEPWTSFAGHTCLEENWKAE

SEQ ID NO: 299 - protein, 80 aa, Homo sapiens
DPVAFKDVAVNFTQEEWALLDISQRKLYREVMLETFRNLTSLGKRWKDQNIEYEHQNPRRNFRSLIEEKVNEIKDDSHCG

SEQ ID NO: 300 - protein, 80 aa, Homo sapiens
SKDLVTFGDVAVNFSQEEWEWLNPAQRNLYRKVMLENYRSLVSLGVSVSKPDVISLLEQGKEPWMVKKEGTRGPCPDWEY

SEQ ID NO: 301 - protein, 80 aa, Homo sapiens
DSVAFEDVAINFTCEEWALLGPSQKSLYRDVMQETIRNLDCIGMIWQNHDIEEDQYKDLRRNLRCHMVERACEIKDNSQC

SEQ ID NO: 302 - protein, 80 aa, Homo sapiens
GPLTFMDVAIEFCLEEWQCLDIAQQNLYRNVMLENYRNLVFLGIAVSKPDLITCLEQEKEPWEPMRRHEMVAKPPVMCSH

SEQ ID NO: 303 - protein, 80 aa, Homo sapiens
QSNDIARGFERGLEPEKIIGATDSCGDLMFLMKWKDTDEADLVLAKEANVKCPQIVIAFYEERLTWHAYPEDAENKEKET

SEQ ID NO: 304 - protein, 80 aa, Homo sapiens
ALPAKDSAWPWEEKPRYLGPVTFEDVAVLFTEAEWKRLSLEQRNLYKEVMLENLRNLVSLAESKPEVHTCPSCPLAFGSQ

SEQ ID NO: 305 - protein, 80 aa, Homo sapiens
DAKSLTAWSRTLVTFKDVFVDFTREEWKLLDTAQQIVYRNVMLENYKNLVSLGYQLTKPDVILRLEKGEEPWLVEREIHQ

SEQ ID NO: 306 - protein, 80 aa, Homo sapiens
DAVAFEDVAVNFTQEEWALLGPSQKNLYRYVMQETIRNLDCIRMIWEEQNTEDQYKNPRRNLRCHMVERFSESKDSSQCG

SEQ ID NO: 307 - protein, 80 aa, Homo sapiens
EKTKVERMVEDYLASGYQDSVTFDDVAVDFTPEEWALLDTTEKYLYRDVMLENYMNLASVEWEIQPRTKRSSLQQGFLKN

SEQ ID NO: 308 - protein, 80 aa, Homo sapiens
DSVAFEDVDVNFTQEEWALLDPSQKNLYRDVMWETMRNLASIGKKWKDQNIKDHYKHRGRNLRSHMLERLYQTKDGSQRG

SEQ ID NO: 309 - protein, 80 aa, Homo sapiens
IESQEPVTFEDVAVDFTQEEWQQLNPAQKTLHRDVMLETYNHLVSVGCSGIKPDVIFKLEHGKDPWIIESELSRWIYPDR

SEQ ID NO: 310 - protein, 80 aa, Homo sapiens
AVGLCKAMSQGLVTFRDVALDFSQEEWEWLKPSQKDLYRDVMLENYRNLVWLGLSISKPNMISLLEQGKEPWMVERKMSQ

SEQ ID NO: 311 - protein, 80 aa, Homo sapiens
DKVEEEAMAPGLPTACSQEPVTFADVAVVFTPEEWVFLDSTQRSLYRDVMLENYRNLASVADQLCKPNALSYLEERGEQW

SEQ ID NO: 312 - protein, 80 aa, Homo sapiens
TFEDVAVYFSQEEWGLLDTAQRALYRHVMLENFTLVTSLGLSTSRPRVVIQLERGEEPWVPSGKDMTLARNTYGRLNSGS

SEQ ID NO: 313 - protein, 80 aa, Homo sapiens
AEPPRLPLTFEDVAIYFSEQEWQDLEAWQKELYKHVMRSNYETLVSLDDGLPKPELISWIEHGGEPFRKWRESQKSGNII

SEQ ID NO: 314 - protein, 80 aa, Homo sapiens
DSVVFEDVAVNFTQEEWALLGPSQKKLYRDVMQETFVNLASIGENWEEKNIEDHKNQGRKLRSHMVERLCERKEGSQFGE

SEQ ID NO: 315 - protein, 80 aa, Homo sapiens
AAAALRDPAQGCVTFEDVTIYFSQEEWVLLDEAQRLLYCDVMLENFALIASLGLISFRSHIVSQLEMGKEPWVPDSVDMT

SEQ ID NO: 316 - protein, 80 aa, Homo sapiens
AAPALALVSFEDVVVTFTGEEWGHLDLAQRTLYQEVMLETCRLLVSLGHPVPKPELIYLLEHGQELWTVKRGLSQSTCAG

SEQ ID NO: 317 - protein, 80 aa, Homo sapiens
IKSQESLTLEDVAVEFTWEEWQLLGPAQKDLYRDVMLENYSNLVSVGYQASKPDALFKLEQGEPWTVENEIHSQICPEIK

SEQ ID NO: 318 - protein, 80 aa, Homo sapiens
GESLESRVTLGSLTAESQELLTFKDVSVDFTQEEWGQLAPAHRNLYREVMLENYGNLVSVGCQLSKPGVISQLEKGEEPW

SEQ ID NO: 319 - protein, 80 aa, Homo sapiens
ASVALEDVAVNFTREEWALLGPCQKNLYKDVMQETIRNLDCVGMKWKDQNIEDQYRYPRKNLRCRMLERFVESKDGTQCG

SEQ ID NO: 320 - protein, 80 aa, Homo sapiens
TESQGTVTFKDVAIDFTQEEWKRLDPAQRKLYRNVMLENYNNLITVGYPFTKPDVIFKLEQEEEPWVMEEEVLRRHWQGE

SEQ ID NO: 321 - protein, 80 aa, Homo sapiens
DSVASEDVAVNFTLEEWALLDPSQKKLYRDVMRETFRNLACVGKKWEDQSIEDWYKNQGRILRNHMEEGLSESKEYDQCG

SEQ ID NO: 322 - protein, 80 aa, Homo sapiens
EETQGELTSSCGSKTMANVSLAFRDVSIDLSQEEWECLDAVQRDLYKDVMLENYSNLVSLGYTIPKPDVITLLEQEKEPW

SEQ ID NO: 323 - protein, 80 aa, Homo sapiens
AAILLTTRPKVPVSFEDVSVYFTKTEWKLLDLRQKVLYKRVMLENYSHLVSLGFSFSKPHLISQLERGEGPWVADIPRTW

SEQ ID NO: 324 - protein, 80 aa, Homo sapiens
KDKVEKEKSEKETTSKKNSHKKTRPRLKNVDRSSAQHLEVTVGDLTVIITDFKEKTKSPPASSAASADQHSQSGSSSDNT

SEQ ID NO: 325 - protein, 80 aa, Homo sapiens
GPLTFTDVAIKFSLEEWQFLDTAQQNLYRDVMLENYRNLVFLGVGVSKPDLITCLEQGKEPWNMKRHKMVAKPPVVCSHF

SEQ ID NO: 326 - protein, 80 aa, Homo sapiens
RKPNPQAMAALFLSAPPQAEVTFEDVAVYLSREEWGRLGPAQRGLYRDVMLETYGNLVSLGVGPAGPKPGVISQLERGDE

SEQ ID NO: 327 - protein, 80 aa, Homo sapiens
LSLSPILLYTCEMFQDPVAFKDVAVNFTQEEWALLDISQKNLYREVMLETFWNLTSIGKKWKDQNIEYEYQNPRRNFRSV

SEQ ID NO: 328 - protein, 80 aa, Homo sapiens
AAGEPRSLLFFQKPVTFEDVAVNFTQEEWDCLDASQRVLYQDVMSETFKNLTSVARIFLHKPELITKLEQEEEQWRETRV

SEQ ID NO: 329 - protein, 80 aa, Homo sapiens
AAMPLKAQYQEMVTFEDVAVHFTKTEWTGLSPAQRALYRSVMLENFGNLTALGYPVPKPALISLLERGDMAWGLEAQDDP

SEQ ID NO: 330 - protein, 80 aa, Homo sapiens
ARVPLTFSDVAIDFSQEEWEYLNSDQRDLYRDVMLENYTNLVSLDFNFTTESNKLSSEKRNYEVNAYHQETWKRNKTFNL

SEQ ID NO: 331 - protein, 80 aa, Homo sapiens
ASRLPTAWSCEPVTFEDVTLGFTPEEWGLLDLKQKSLYREVMLENYRNLVSVEHQLSKPDVVSQLEEAEDFWPVERGIPQ

SEQ ID NO: 332 - protein, 80 aa, Homo sapiens
SKKKRDAADKPRGFARGLDPERIIGATDSSGELMFLMKWKDSDEADLVLAKEANMKCPQIVIAFYEERLTWHSCPEDEAQ

SEQ ID NO: 333 - protein, 80 aa, Homo sapiens
AHKYVGLQYHGSVTFEDVAIAFSQQEWESLDSSQRGLYRDVMLENYRNLVSMGHSRSKPHVIALLEQWKEPEVTVRKDGR

SEQ ID NO: 334 - protein, 80 aa, Homo sapiens
AAARLLPVPAGPQPLSFQAKLTFEDVAVLLSQDEWDRLCPAQRGLYRNVMMETYGNVVSLGLPGSKPDIISQLERGEDPW

SEQ ID NO: 335 - protein, 80 aa, Homo sapiens
AVGLLKAMYQELVTFRDVAVDFSQEEWDCLDSSQRHLYSNVMLENYRILVSLGLCFSKPSVILLLEQGKAPWMVKRELTK

SEQ ID NO: 336 - protein, 80 aa, Homo sapiens
GLLTFRDVAIEFSLEEWQCLDTAQRNLYKNVILENYRNLVFLGIAVSKQDLITCLEQEKEPLTVKRHEMVNEPPVMCSHF

SEQ ID NO: 337 - protein, 80 aa, Homo sapiens
GLLAFRDVALEFSPEEWECLDPAQRSLYRDVMLENYRNLISLGEDSFNMQFLFHSLAMSKPELIICLEARKEPWNVNTEK

SEQ ID NO: 338 - protein, 80 aa, Homo sapiens
NLTEGRVVFEDVAIYFSQEEWGHLDEAQRLLYRDVMLENLALLSSLGSWHGAEDEEAPSQQGFSVGVSEVTASKPCLSSQ

SEQ ID NO: 339 - protein, 80 aa, Homo sapiens
GPAQHTSWPCGSAVPTLKSMVTFEDVAVYFSQEEWELLDAAQRHLYHSVMLENLELVTSLGSWHGVEGEGAHPKQNVSVE

SEQ ID NO: 340 - protein, 80 aa, Homo sapiens
SGYPGAERNLLEYSYFEKGPLTFRDVVIEFSQEEWQCLDTAQQDLYRKVMLENFRNLVFLGIDVSKPDLITCLEQGKDPW

SEQ ID NO: 341 - protein, 80 aa, Homo sapiens
ARKLVMFRDVAIDFSQEEWECLDSAQRDLYRDVMLENYSNLVSLDLPSRCASKDLSPEKNTYETELSQWEMSDRLENCDL

SEQ ID NO: 342 - protein, 80 aa, Homo sapiens
GPLQFRDVAIEFSLEEWHCLDTAQRNLYRNVMLENYSNLVFLGITVSKPDLITCLEQGRKPLTMKRNEMIAKPSVMCSHF

SEQ ID NO: 343 - protein, 80 aa, Homo sapiens
VAGWLTNYSQDSVTFEDVAVDFTQEEWTLLDQTQRNLYRDVMLENYKNLVAVDWESHINTKWSAPQQNFLQGKTSSVVEM

SEQ ID NO: 344 - protein, 80 aa, Homo sapiens
AAAWMAPAQESVTFEDVAVTFTQEEWGQLDVTQRALYVEVMLETCGLLVALGDSTKPETVEPIPSHLALPEEVSLQEQLA

SEQ ID NO: 345 - protein, 80 aa, Homo sapiens
VLEWLFISQEQPKITKSWGPLSFMDVFVDFTWEEWQLLDPAQKCLYRSVMLENYSNLVSLGYQHTKPDIIFKLEQGEELC

SEQ ID NO: 346 - protein, 80 aa, Homo sapiens
AAAVLMDRVQSCVTFEDVFVYFSREEWELLEEAQRFLYRDVMLENFALVATLGFWCEAEHEAPSEQSVSVEGVSQVRTAE

SEQ ID NO: 347 - protein, 80 aa, Homo sapiens
AGSQFPDFKHLGTFLVFEELVTFEDVLVDFSPEELSSLSAAQRNLYREVMLENYRNLVSLGHQFSKPDIISRLEEEESYA

SEQ ID NO: 348 - protein, 80 aa, Homo sapiens
IQSQISFEDVAVDFTLEEWQLLNPTQKNLYRDVMLENYSNLVFLEVWLDNPKMWLRDNQDNLKSMERGHKYDVFGKIFNS

SEQ ID NO: 349 - protein, 80 aa, Homo sapiens
DLVAFEDVAVNFTQEEWSLLDPSQKNLYREVMQETLRNLASIGEKWKDQNIEDQYKNPRNNLRSLLGERVDENTEENHCG

SEQ ID NO: 350 - protein, 80 aa, Homo sapiens
KDEDIKLRVIGQDSSEIHFKVKMTTPLKKLKKSYCQRQGVPVNSLRFLFEGQRIADNHTPEELGMEEEDVIEVYQEQIGG

SEQ ID NO: 351 - protein, 80 aa, Homo sapiens
DSVAFEDVAVNFTQEEWALLSPSQKNLYRDVTLETFRNLASVGIQWKDQDIENLYQNLGIKLRSLVERLCGRKEGNEHRE

SEQ ID NO: 352 - protein, 80 aa, Homo sapiens
RNFWILRLPPGSKGEAPKVPVTFDDVAVYFSELEWGKLEDWQKELYKHVMRGNYETLVSLDYAISKPDILTRIERGEEPC

SEQ ID NO: 353 - protein, 80 aa, Homo sapiens
AAAAPRRPTQQGTVTFEDVAVNFSQEEWCLLSEAQRCLYRDVMLENLALISSLGCWCGSKDEEAPCKQRISVQRESQSRT

SEQ ID NO: 354 - protein, 80 aa, Homo sapiens
SGEDSGSFYSWQKAKREQGLVTFEDVAVDFTQEEWQYLNPPQRTLYRDVMLETYSNLVFVGQQVTKPNLILKLEVEECPA

SEQ ID NO: 355 - protein, 80 aa, Homo sapiens
DSVAFEDVAVNFTQEEWALLGPSQKSLYRNVMQETIRNLDCIEMKWEDQNIGDQCQNAKRNLRSHTCEIKDDSQCGETFG

SEQ ID NO: 356 - protein, 80 aa, Homo sapiens
AAGWLTTWSQNSVTFQEVAVDFSQEEWALLDPAQKNLYKDVMLENFRNLASVGYQLCRHSLISKVDQEQLKTDERGILQG

SEQ ID NO: 357 - protein, 80 aa, Homo sapiens
ENPRNQLMALMLLTAQPQELVMFEEVSVCFTSEEWACLGPIQRALYWDVMLENYGNVTSLEWETMTENEEVTSKPSSSQR

SEQ ID NO: 358 - protein, 80 aa, Homo sapiens
LETYNSLVSLQELVSFEEVAVHFTWEEWQDLDDAQRTLYRDVMLETYSSLVSLGHCITKPEMIFKLEQGAEPWIVEETPN

SEQ ID NO: 359 - protein, 80 aa, Homo sapiens
RHFRRPEPCREPLASPIQDSVAFEDVAVNFTQEEWALLDSSQKNLYREVMQETCRNLASVGSQWKDQNIEDHFEKPGKDI

SEQ ID NO: 360 - protein, 80 aa, Homo sapiens
AAAELTAPAQGIVTFEDVAVYFSWKEWGLLDEAQKCLYHDVMLENLTLTTSLGGSGAGDEEAPYQQSTSPQRVSQVRIPK

SEQ ID NO: 361 - protein, 80 aa, Homo sapiens
AATFQLPGHQEMPLTFQDVAVYFSQAEGRQLGPQQRALYRDVMLENYGNVASLGFPVPKPELISQLEQGKELWVLNLLGA

SEQ ID NO: 362 - protein, 80 aa, Homo sapiens
RSEAGEPPSSLQVKPETPASAAVAVAAAAAPTTTAEKPPAEAQDEPAESLSEFKPFFGNIIITDVTANCLTVTFKEYVTV

SEQ ID NO: 363 - protein, 80 aa, Homo sapiens
HRTTRIKITELNPHLMCALCGGYFIDATTIVECLHSFCKTCIVRYLETNKYCPMCDVQVHKTRPLLSIRSDKTLQDIVYK

SEQ ID NO: 364 - protein, 80 aa, Homo sapiens
ASQEFEVEAIVDKRQDKNGNTQYLVRWKGYDKQDDTWEPEQHLMNCEKCVHDFNRRQTEKQKKLTWTTTSRIFSNNARRR

SEQ ID NO: 365 - protein, 80 aa, Homo sapiens
ASGDLYEVERIVDKRKNKKGKWEYLIRWKGYGSTEDTWEPEHHLLHCEEFIDEFNGLHMSKDKRIKSGKQSSTSKLLRDS

SEQ ID NO: 366 - protein, 80 aa, Homo sapiens
TLIRKADLENHNKDGGFWTVIDGKVYDIKDFQTQSLTGNSILAQFAGEDPVVALEAALQFEDTRESMHAFCVGQYLEPDQ

SEQ ID NO: 367 - protein, 80 aa, Homo sapiens
EKTKIGTMVEDHRSNSYQDSVTFDDVAVEFTPEEWALLDTTQKYLYRDVMLENYMNLASVDFFFCLTSEWEIQPRTKRSS

SEQ ID NO: 368 - protein, 80 aa, Homo sapiens
AHELVMFRDVAIDVSQEEWECLNPAQRNLYKEVMLENYSNLVSLGLSVSKPAVISSLEQGKEPWMVVREETGRWCPGTWK

SEQ ID NO: 369 - protein, 80 aa, Homo sapiens
AFEDVAVYFSQEEWGLLDTAQRALYRRVMLDNFALVASLGLSTSRPRVVIQLERGEEPWVPSGTDTTLSRTTYRRRNPGS

SEQ ID NO: 370 - protein, 80 aa, Homo sapiens
AQLRRGHLTFRDVAIEFSQEEWKCLDPVQKALYRDVMLENYRNLVSLGICLPDLSIISMMKQRTEPWTVENEMKVAKNPD

SEQ ID NO: 371 - protein, 80 aa, Homo sapiens
SDHSLGISRSKTPVDDPMSLLYNMNDCYSKLKELVPSIPQNKKVSKMEILQHVIDYILDLQIALDSHPTIVSLHHQRPGQ

SEQ ID NO: 372 - protein, 80 aa, Homo sapiens
KDPNEPQKPVSAYALFFRDTQAAIKGQNPNATFGEVSKIVASMWDGLGEEQKQVYKKKTEAAKKEYLKQLAAYRASLVSK

SEQ ID NO: 373 - protein, 80 aa, Homo sapiens
QEEKQEDAAICPVTVLPEEPVTFQDVAVDFSREEWGLLGPTQRTEYRDVMLETFGHLVSVGWETTLENKELAPNSDIPEE

SEQ ID NO: 374 - protein, 80 aa, Homo sapiens
DASRLSGRDPSSWTVEDVMQFVREADPQLGPHADLFRKHEIDGKALLLLRSDMMMKYMGLKLGPALKLSYHIDRLKQGKF

SEQ ID NO: 375 - protein, 80 aa, Homo sapiens
AVTFEDVTIIFTWEEWKFLDSSQKRLYREVMWENYTNVMSVENWNESYKSQEEKFRYLEYENFSYWQGWWNAGAQMYENQ

SEQ ID NO: 376 - protein, 80 aa, Homo sapiens
ELSAIGEQVFAVESIRKKRVRKGKVEYLVKWKGWPPKYSTWEPEEHILDPRLVMAYEEKEERDRASGYRKRGPKPKRLLL

SEQ ID NO: 377 - protein, 80 aa, Homo sapiens
GGAGARLPALLDEQQVNVLLYDMNGCYSRLKELVPTLPQNRKVSKVEILQHVIDYIRDLQLELNSESEVGTPGGRGLPVR

SEQ ID NO: 378 - protein, 80 aa, Homo sapiens
VVMAASPGSLHSPQQLAEEATRKRELRLMKNREAAKECRRRKKEYVKCLESRVAVLEVQNKKLIEELETLKDICSPKTDY

SEQ ID NO: 379 - protein, 80 aa, Homo sapiens
GGGPGGRPGREPRQRHTANARERDRTNSVNTAFTALRTLIPTEPADRKLSKIETLRLASSYISHLGNVLLAGEACGDGQP

SEQ ID NO: 380 - protein, 80 aa, Homo sapiens
SGFGYSLPQQQPAAVARRNERERNRVKLVNLGFATLREHVPNGAANKKMSKVETLRSAVEYIRALQQLLDEHDAVSAAFQ

SEQ ID NO: 381 - protein, 80 aa, Homo sapiens
APLPPRDPNGAGPEWREPGAVSFADVAVYFCREEWGCLRPAQRALYRDVMRETYGHLSALGIGGNKPALISWVEEEAELW

SEQ ID NO: 382 - protein, 80 aa, Homo sapiens
KQGFSKDPSTWSVDEVIQFMKHTDPQISGPLADLFRQHEIDGKALFLLKSDVMMKYMGLKLGPALKLCYYIEKLKEGKYS

SEQ ID NO: 383 - protein, 80 aa, Homo sapiens
SGGGSPQSYEELQTQRVMANVRERQRTQSLNEAFAALRKIIPTLPSDKLSKIQTLKLAARYIDFLYQVLQSDELDSKMAS

SEQ ID NO: 384 - protein, 80 aa, Homo sapiens
IAPGVVMASSPALPTQPAEEAARKREVRLMKNREAARECRRKKKEYVKCLENRVAVLENQNKTLIEELKALKDLYCHKSD

SEQ ID NO: 385 - protein, 80 aa, Homo sapiens
SRIPVSKSQPVTPEKHRARKRQAWLWEEDKNLRSGVRKYGEGNWSKILLHYKFNNRTSVMLKDRWRTMKKLKLISSDSED

SEQ ID NO: 386 - protein, 80 aa, Homo sapiens
SLAIARGRGKGPAAEEPLSLLDDMNHCYSRLRELVPGVPRGTQLSQVEILQRVIDYILDLQVVLAEPAPGPPDGPHLPIQ

SEQ ID NO: 387 - protein, 80 aa, Homo sapiens
GSGPPSSGGGLYRDMGAQGGRPSLIARIPVARILGDPEEESWSPSLTNLEKVVVTDVTSNFLTVTIKESNTDQGFFKEKR

SEQ ID NO: 388 - protein, 80 aa, Homo sapiens
ELPAVGEHVFAVESIEKKRIRKGRVEYLVKWRGWSPKYNTWEPEENILDPRLLIAFQNRERQEQLMGYRKRGPKPKPLVV

SEQ ID NO: 389 - protein, 80 aa, Homo sapiens
VDSSSNQLPSSKRMRTAFTSTQLLELEREFASNMYLSRLRRIEIATYLNLSEKQVKIWFQNRRVKHKKEGKGSNHRGGGG

SEQ ID NO: 390 - protein, 80 aa, Homo sapiens
TPGGGGDAGKKRKRRVLFSKAQTYELERRFRQQRYLSAPEREHLASLIRLTPTQVKIWFQNHRYKMKRARAEKGMEVTPL

SEQ ID NO: 391 - protein, 80 aa, Homo sapiens
QTVVMTSPVTLTSQTTKTDDPQLKREIRLMKNREAARECRRKKKEYVKCLENRVAVLENQNKTLIEELKTLKDLYSNKSV

SEQ ID NO: 392 - protein, 80 aa, Homo sapiens
KGSPSAQSFEELQSQRILANVRERQRTQSLNEAFAALRKIIPTLPSDKLSKIQTLKLAARYIDFLYQVLQSDEMDNKMTS

SEQ ID NO: 393 - protein, 80 aa, Homo sapiens
NLTEDYMVFEDVAIHFSQEEWGILNDVQRHLHSDVMLENFALLSSVGCWHGAKDEEAPSKQCVSVGVSQVTTLKPALSTQ

SEQ ID NO: 394 - protein, 80 aa, Homo sapiens
KDPNEPQKPVSAYALFFRDTQAAIKGQNPNATFGEVSKIVASMWDSLGEEQKQVYKRKTEAAKKEYLKALAAYRASLVSK

SEQ ID NO: 395 - protein, 80 aa, Homo sapiens
KDPNEPQKPVSAYALFFRDTQAAIKGQNPNATFGEVSKIVASMWDSLGEEQKQVYKRKTEAAKKEYLKALAAYKDNQECQ

SEQ ID NO: 396 - protein, 80 aa, Homo sapiens
LDGSTWDFCSEDCKSKYLLWYCKAARCHACKRQGKLLETIHWRGQIRHFCNQQCLLRFYSQQNQPNLDTQSGPESLLNSQ

SEQ ID NO: 397 - protein, 80 aa, Homo sapiens
ASVQASRRQWCYLCDLPKMPWAMVWDFSEAVCRGCVNFEGADRIELLIDAARQLKRSHVLPEGRSPGPPALKHPATKDLA

SEQ ID NO: 398 - protein, 80 aa, Homo sapiens
MEGPQPENMQPRTRRTKFTLLQVEELESVFRHTQYPDVPTRRELAENLGVTEDKVRVWFKNKRARCRRHQRELMLANELR

SEQ ID NO: 399 - protein, 80 aa, Homo sapiens
PKIMPKKPAEEGNDSEEVPEASGPQNDGKELCPPGKPTTSEKIHERSGPKRGEHAWTHRLRERKQLVIYEEISDPEEDDE

SEQ ID NO: 400 - protein, 80 aa, Homo sapiens
SAAQVSSSRRQSCYLCDLPRMPWAMIWDFSEPVCRGCVNYEGADRIEFVIETARQLKRAHGCFQDGRSPGPPPPVGVKTV

SEQ ID NO: 401 - protein, 80 aa, Homo sapiens
PGPPGSLEMGPLTFRDVAIEFSLEEWQCLDTAQRNLYRKVMFENYRNLVFLGIAVSKPHLITCLEQGKEPWNRKRQEMVA

SEQ ID NO: 402 - protein, 80 aa, Homo sapiens
NKKKVEEVLEEEEEEYVVEKVLDRRVVKGKVEYLLKWKGFSDEDNTWEPEENLDCPDLIAEFLQSQKTAHETDKSEGGKR

SEQ ID NO: 403 - protein, 80 aa, Homo sapiens
LANVVEKVRLLRLHPGMGLKGDLCERHGEKLKMFCKEDVLIMCEACSQSPEHEAHSVVPMEDVAWEYKWELHEALEHLKK

SEQ ID NO: 404 - protein, 80 aa, Homo sapiens [sequence omitted]

SEQ ID NO: 405 - protein, 80 aa, Homo sapiens
ENSDLLPVAQTEPSIWTVDDVWAFIHSLPGCQDIADEFRAQEIDGQALLLLKEDHLMSAMNIKLGPALKICARINSLKES

SEQ ID NO: 406 - protein, 80 aa, Homo sapiens
AGPGGGSRSGSGRPAAANAARERSRVQTLRHAFLELQRTLPSVPPDTKLSKLDVLLLATTYIAHLTRSLQDDAEAPADAG

SEQ ID NO: 407 - protein, 80 aa, Homo sapiens
QNGKSKKVEEAEPEEFVVEKVLDRRVVNGKVEYFLKWKGFTDADNTWEPEENLDCPELIEAFLNSQKAGKEKDGTKRKSL

SEQ ID NO: 408 - protein, 80 aa, Homo sapiens
QHPPDACAFRRGRKKRIPYSKGQLRELEREYAANKFITKDKRRKISAATSLSERQITIWFQNRRVKEKKVLAKVKNSATP

SEQ ID NO: 409 - protein, 80 aa, Homo sapiens
SMSPTTSSQILARKRRRGIIEKRRRDRINNSLSELRRLVPSAFEKQGSAKLEKAEILQMTVDHLKMLHTAGGKGYFDAHA

SEQ ID NO: 410 - protein, 80 aa, Homo sapiens
LVGMGHHFLPSEPTKWNVEDVYEFIRSLPGCQEIAEEFRAQEIDGQALLLLKEDHLMSAMNIKLGPALKIYARISMLKDS

SEQ ID NO: 411 - protein, 80 aa, Homo sapiens
PANEDAPQPGEHGSACEVSVSFEDVTVDFSREEWQQLDSTQRRLYQDVMLENYSHLLSVGFEVPKPEVIFKLEQGEGPWT

SEQ ID NO: 412 - protein, 80 aa, Homo sapiens
GYSSTENLQLVLERRRVANAKERERIKNLNRGFARLKALVPFLPQSRKPSKVDILKGATEYIQVLSDLLEGAKDSKKQDP

SEQ ID NO: 413 - protein, 80 aa, Homo sapiens
EEAPAPEDVTKWTVDDVCSFVGGLSGCGEYTRVFREQGIDGETLPLLTEEHLLTNMGLKLGPALKIRAQVARRLGRVFYV

SEQ ID NO: 414 - protein, 80 aa, Homo sapiens
GGTLAHTPRRSLPSHHGKKMRMARCGHCRGCLRVQDCGSCVNCLDKPKFGGPNTKKQCCVYRKCDKIEARKMERLAKKGR

SEQ ID NO: 415 - protein, 80 aa, Homo sapiens
LNSPTTTSQIMARKKRRGIIEKRRRDRINNSLSELRRLVPTAFEKQGSAKLEKAEILQMTVDHLKMLQATGGKGYFDAHA

SEQ ID NO: 416 - protein, 80 aa, Homo sapiens
QPVKSELDEEEERRKRRREKNKVAAARCRNKKKERTEFLQRESERLELMNAELKTQIEELKQERQQLILMLNRHRPTCIV

SEQ ID NO: 417 - protein, 80 aa, Homo sapiens
LQPEVSSYRRGRKKRVPYTKVQLKELEKEYAASKFITKEKRRRISATTNLSERQVTIWFQNRRVKEKKVVSKSKAPHLHS

SEQ ID NO: 418 - protein, 80 aa, Homo sapiens
LPVPLDSAFEPAFLRKRNERERQRVRCVNEGYARLRDHLPRELADKRLSKVETLRAAIDYIKHLQELLERQAWGLEGAAG

SEQ ID NO: 419 - protein, 80 aa, Homo sapiens
SPFLQRPLHKRKGGQVRFSNDQTIELEKKFETQKYLSPPERKRLAKMLQLSERQVKTWFQNRRAKWRRLKQENPQSNKKE

SEQ ID NO: 420 - protein, 80 aa, Homo sapiens
IAIATGSLHCVCCTEDGEVYTWGDNDEGQLGDGTTNAIQRPRLVAALQGKKVNRVACGSAHTLAWSTSKPASAGKLPAQV

SEQ ID NO: 421 - protein, 80 aa, Homo sapiens
GGSDASQVPNGKRMRTAFTSTQLLELEREFSSNMYLSRLRRIEIATYLNLSEKQVKIWFQNRRVKHKKEGKGTQRNSHAG

SEQ ID NO: 422 - protein, 80 aa, Homo sapiens
RLDLPPGFMFKVQAQHDYTATDTDELQLKAGDVVLVIPFQNPEEQDEGWLMGVKESDWNQHKELEKCRGVFPENFTERVP

SEQ ID NO: 423 - protein, 80 aa, Homo sapiens
GICKLPGRLRIQPALWSREDVLHWLRWAEQEYSLPCTAEHGFEMNGRALCILTKDDFRHRAPSSGDVLYELLQYIKTQRR

SEQ ID NO: 424 - protein, 80 aa, Homo sapiens
PNYRGCEYSYGPAFTRKRNERERQRVKCVNEGYAQLRHHLPEEYLEKRLSKVETLRAAIKYINYLQSLLYPDKAETKNNP

SEQ ID NO: 425 - protein, 80 aa, Homo sapiens
LHGINPVFLSSNPSRWSVEEVYEFIASLQGCQEIAEEFRSQEIDGQALLLLKEEHLMSAMNIKLGPALKICAKINVLKET

SEQ ID NO: 426 - protein, 80 aa, Homo sapiens
QAGQQQGQQKQKRHRTRFTPAQLNELERSFAKTHYPDIFMREELALRIGLTESRVQVWFQNRRAKWKKRKKTTNVFRAPG

SEQ ID NO: 427 - protein, 80 aa, Homo sapiens
AAAVAVAAASRRQSCYLCDLPRMPWAMIWDFTEPVCRGCVNYEGADRVEFVIETARQLKRAHGCFPEGRSPPGAAASAAA

SEQ ID NO: 428 - protein, 80 aa, Homo sapiens
FSSQTPASIKEEEGSPEKERPPEAEYINSRCVLFTYFQGDISSVVDEHFSRALSQPSSYSPSCTSSKAPRSSGPWRDCSF

SEQ ID NO: 429 - protein, 80 aa, Homo sapiens
DKAGGSSGQRTRKKRCPYTKYQIRELEREFFFSVYINKEKRLQLSRMLNLTDRQVKIWFQNRRMKEKKINRDRLQYYSAN

SEQ ID NO: 430 - protein, 80 aa, Homo sapiens
GAPLSGLQGLPECTRCGHGIVGTIVKARDKLYHPECFMCSDCGLNLKQRGYFFLDERLYCESHAKARVKPPEGYDVVAVY

SEQ ID NO: 431 - protein, 80 aa, Homo sapiens
RRPATAETGGGAAAVARRNERERNRVKLVNLGFQALRQHVPHGGASKKLSKVETLRSAVEYIRALQRLLAEHDAVRNALA

SEQ ID NO: 432 - protein, 80 aa, Homo sapiens
TVQVTGKTRTKEKYRVVYTDHQRLELEKEFHCNRYITIQRKSELAVNLGLSERQVKIWFQNRRAKERKMIKKKISQFENS

SEQ ID NO: 433 - protein, 80 aa, Homo sapiens
EEAAQKRKEKEPGMALPQGHLTFRDVAIEFSLEEWKCLDPTQRALYRAMMLENYRNLHSVDISSKCMMKKFSSTAQGNTE

SEQ ID NO: 434 - protein, 80 aa, Homo sapiens
DIRASQVARWTVDEVAEFVQSLLGCEEHAKCFKKEQIDGKAFLLLTQTDIVKVMKIKLGPALKIYNSILMFRHSQELPEE

SEQ ID NO: 435 - protein, 80 aa, Homo sapiens
LSPLEGTKMTVNNLHPRVTEEDIVELFCVCGALKRARLVHPGVAEVVFVKKDDAITAYKKYNNRCLDGQPMKCNLHMNGN

SEQ ID NO: 436 - protein, 80 aa, Homo sapiens
DNAERPRARRRRKPRVLFSQAQVYELERRFKQQRYLSAPERDQLASVLKLTSTQVKIWFQNRRYKCKRQRQDQTLELVGL

SEQ ID NO: 437 - protein, 80 aa, Homo sapiens
SQVKSKAKKTVDKHSDEYKIRRERNNIAVRKSRDKAKMRNLETQHKVLELTAENERLQKKVEQLSRELSTLRNLFKQLPE

SEQ ID NO: 438 - protein, 80 aa, Homo sapiens
KRDYIRLYGIKCAKCSIGFSKNDFVMRARSKVYHIECFRCVACSRQLIPGDEFALREDGLFCRADHDVVERASLGAGDPL

SEQ ID NO: 439 - protein, 80 aa, Homo sapiens
SLGSQVKTRTKDKYRVVYTDHQRLELEKEFHYSRYITIRRKAELAATLGLSERQVKIWFQNRRAKERKINKKKLQQQQQQ

SEQ ID NO: 440 - protein, 80 aa, Homo sapiens
QGGQRGRPHSRRRHRTTFSPVQLEQLESAFGRNQYPDIWARESLARDTGLSEARIQVWFQNRRAKQRKQERSLLQPLAHL

SEQ ID NO: 441 - protein, 80 aa, Homo sapiens
DALTYLDQVKIRFGSDPATYNGFLEIMKEFKSQSIDTPGVIRRVSQLFHEHPDLIVGFNAFLPLGYRIDIPKNGKLNIQS

SEQ ID NO: 442 - protein, 80 aa, Homo sapiens
RLHLDSNPLKWSVADVVRFIRSTDCAPLARIFLDQEIDGQALLLLTLPTVQECMDLKLGPAIKLCHHIERIKFAFYEQFA

SEQ ID NO: 443 - protein, 80 aa, Homo sapiens
AKGAAPNAPRTRKKRCPYSKFQIRELEREFFFNVYINKEKRLQLSRMLNLTDRQVKIWFQNRRMKEKKLSRDRLQYFSGN

SEQ ID NO: 444 - protein, 80 aa, Homo sapiens
TTGNWLTAKSGRKKRCPYTKHQTLELEKEFLFNMYLTRERRLEISKTINLTDRQVKIWFQNRRMKLKKMNRENRIRELTS

SEQ ID NO: 445 - protein, 80 aa, Homo sapiens
YLVSNVIELLDVDPNDQEEDGANIDLDSQRKGKCAVIKTSTRQTYFLPVIGLVDAEKLKPGDLVGVNKDSYLILETLPTE

SEQ ID NO: 446 - protein, 80 aa, Homo sapiens
KASPTLGKRKKRRHRTVFTAHQLEELEKAFSEAHYPDVYAREMLAVKTELPEDRIQVWFQNRRAKWRKREKRWGGSSVMA

SEQ ID NO: 447 - protein, 80 aa, Homo sapiens
EESERPKPRSRRKPRVLFSQAQVFELERRFKQQRYLSAPEREHLASSLKLTSTQVKIWFQNRRYKCKRQRQDKSLELGAH

SEQ ID NO: 448 - protein, 80 aa, Homo sapiens
VVPGSRQEEVIDHKLTEREWAEEWKHLNNLLNCIMDMVEKTRRSLTVLRRCQEADREELNHWARRYSDAEDTKKGPAPAA

SEQ ID NO: 449 - protein, 80 aa, Homo sapiens
ESPEKKPACRKKKTRTVFSRSQVFQLESTFDMKRYLSSSERAGLAASLHLTETQVKIWFQNRRNKWKRQLAAELEAANLS

SEQ ID NO: 450 - protein, 80 aa, Homo sapiens
RGGVGVGGGRKKKTRTVFSRSQVFQLESTFDLKRYLSSAERAGLAASLQLTETQVKIWFQNRRNKWKRQLAAELEAASLS

SEQ ID NO: 451 - protein, 80 aa, Homo sapiens
ELLAHGRQKILDLLNEGSARDLRSLQRIGPKKAQLIVGWRELHGPFSQVEDLERVEGITGKQMESFLKANILGLAAGQRC

SEQ ID NO: 452 - protein, 80 aa, Homo sapiens
ESPYGETISPEDAPESISKAVASLPPEQMFELMKQMKLCVQNSPQEARNMLLQNPQLAYALLQAQVVMRIVDPEIALKIL

SEQ ID NO: 453 - protein, 80 aa, Homo sapiens
AGPLHKGKKAVNKDSLEYRLRRERNNIAVRKSRDKAKRRILETQQKVLEYMAENERLRSRVEQLTQELDTLRNLFRQIPE

SEQ ID NO: 454 - protein, 80 aa, Homo sapiens
IRIVNGKPKKVRKPRTIYSSFQLAALQRRFQKTQYLALPERAELAASLGLTQTQVKIWFQNRRSKFKKMWKSGEIPSEQH

SEQ ID NO: 455 - protein, 80 aa, Homo sapiens
TVYQFCSPSCWTKFQRTSPEGGIHLSCHYCHSLFSGKPEVLDWQDQVFQFCCRDCCEDFKRLRGVVSQCEHCRQEKLLHE

SEQ ID NO: 456 - protein, 80 aa, Homo sapiens
TMVDTEMPFWPTNFGISSVDLSVMEDHSHSFDIKPFTTVDFSSISTPHYEDIPFTRTDPVVADYKYDLKLQEYQSAIKVE

SEQ ID NO: 457 - protein, 80 aa, Homo sapiens
GRHHAELLKPRCSACDEIIFADECTEAEGRHWHMKHFCCLECETVLGGQRYIMKDGRPFCCGCFESLYAEYCETCGEHIG

SEQ ID NO: 458 - protein, 80 aa, Homo sapiens
DPDKESPGCKRRRTRTNFTGWQLEELEKAFNESHYPDVFMREALALRLDLVESRVQVWFQNRRAKWRKKENTKKGPGRPA

SEQ ID NO: 459 - protein, 80 aa, Homo sapiens
TEQPTPRQKKPRRSRTIFTELQLMGLEKKFQKQKYLSTPDRLDLAQSLGLTQLQVKTWYQNRRMKWKKMVLKGGQEAPTK

SEQ ID NO: 460 - protein, 80 aa, Homo sapiens
SMELAKNKSKKRRNRTTFSTFQLEELEKVFQKTHYPDVYAREQLALRTDLTEARVQVWFQNRRAKWRKRERYGKIQEGRN

SEQ ID NO: 461 - protein, 80 aa, Homo sapiens
GGGGGAGPVVVVRQRQAANARERDRTQSVNTAFTALRTLIPTEPVDRKLSKIETVRLASSYIAHLANVLLLGDSADDGQP

SEQ ID NO: 462 - protein, 80 aa, Homo sapiens
IDDTVEGITGNLFEVYLKPYFLEAYRPIRKGDIFLVRGGMRAVEFKVVETDPSPYCIVAPDTVIHCEGEPIKREDEEESL

SEQ ID NO: 463 - protein, 80 aa, Homo sapiens
SALNQTKKRKKRRHRTIFTSYQLEELEKAFNEAHYPDVYAREMLAMKTELPEDRIQVWFQNRRAKWRKREKCWGRSSVMA

SEQ ID NO: 464 - protein, 80 aa, Homo sapiens
DGLPWGAAPGRARKKRKPYTKQQIAELENEFLVNEFINRQKRKELSNRLNLSDQQVKIWFQNRRMKKKRVVLREQALALY

SEQ ID NO: 465 - protein, 80 aa, Homo sapiens
GGGGSGKTRTKDKYRVVYTDHQRLELEKEFHYSRYITIRRKSELAANLGLTERQVKIWFQNRRAKERKVNKKKQQQQQPP

SEQ ID NO: 466 - protein, 80 aa, Homo sapiens
TRAGGLALGRSEASPENAARERSRVRTLRQAFLALQAALPAVPPDTKLSKLDVLVLAASYIAHLTRTLGHELPGPAWPPF

SEQ ID NO: 467 - protein, 80 aa, Homo sapiens
KCDSNVSSSKKRRHRTTFTSLQLEELEKVFQKTHYPDVYVREQLALRTELTEARVQVWFQNRRAKWRKRERYGQIQQAKS

SEQ ID NO: 468 - protein, 80 aa, Homo sapiens
NAANWLTAKSGRKKRCPYTKHQTLELEKEFLFNMYLTRERRLEISRSVHLTDRQVKIWFQNRRMKLKKMNRENRIRELTA

SEQ ID NO: 469 - protein, 80 aa, Homo sapiens
LSEEEQPKKKHRRNRTTFTTYQLHELERAFEKSHYPDVYSREELAGKVNLPEVRVQVWFQNRRAKWRRQEKLEVSSMKLQ

SEQ ID NO: 470 - protein, 80 aa, Homo sapiens
HMAGLAEYPMQGELASAISSGKKKRKRCGMCAPCRRRINCEQCSSCRNRKTGHQICKFRKCEELKKKPSAALEKVMLPTG

SEQ ID NO: 471 - protein, 80 aa, Homo sapiens
SITKHPSTWSVEAVVLFLKQTDPLALCPLVDLFRSHEIDGKALLLLTSDVLLKHLGVKLGTAVKLCYYIDRLKQGKCFEN

SEQ ID NO: 472 - protein, 80 aa, Homo sapiens
ACRRKREFIPDEKKDAMYWEKRRKNNEAAKRSREKRRLNDLVLENKLIALGEENATLKAELLSLKLKFGLISSTAYAQEI

SEQ ID NO: 473 - protein, 80 aa, Homo sapiens
EIRFNGKGKKIRKPRTIYSSLQLQALNHRFQQTQYLALPERAELAASLGLTQTQVKIWFQNKRSKFKKLLKQGSNPHESD

SEQ ID NO: 474 - protein, 80 aa, Homo sapiens
GLHGTRQEEMIDHRLTDREWAEEWKHLDHLLNCIMDMVEKTRRSLTVLRRCQEADREELNYWIRRYSDAEDLKKGGGSSS

SEQ ID NO: 475 - protein, 80 aa, Homo sapiens
ELSAVGERVFAAEALLKRRIRKGRMEYLVKWKGWSQKYSTWEPEENILDARLLAAFEEREREMELYGPKKRGPKPKTFLL

SEQ ID NO: 476 - protein, 80 aa, Homo sapiens
AREKSAGKRGPDRGSPEYRQRRERNNIAVRKSRDKAKRRNQEMQQKLVELSAENEKLHQRVEQLTRDLAGLRQFFKQLPS

SEQ ID NO: 477 - protein, 80 aa, Homo sapiens
SGGCDNLIKLWKEEEDGQWKEEQKLEAHSDWVRDVAWAPSIGLPTSTIASCSQDGRVFIWTCDDASSNTWSPKLLHKFND

SEQ ID NO: 478 - protein, 80 aa, Homo sapiens
VKGVDLDAPGSINGVPLLEVDLDSFEDKPWRKPGADLSDYFNYGFNEDTWKAYCEKQKRIRMGLEVIPVTSTTNKITAED

SEQ ID NO: 479 - protein, 80 aa, Homo sapiens
KADSESNKGKKRRNRTTFTSYQLEELEKVFQKTHYPDVYAREQLAMRTDLTEARVQVWFQNRRAKWRKRERFGQMQQVRT

SEQ ID NO: 480 - protein, 80 aa, Homo sapiens
TAKQREAEATAKRPRTTITAKQLETLKSAYNTSPKPARHVREQLSSETGLDMRVVQVWFQNRRAKEKRLKKDAGRQRWGQ

SEQ ID NO: 481 - protein, 80 aa, Homo sapiens
GRHHAECLKPRCAACDEIIFADECTEAEGRHWHMKHFCCFECETVLGGQRYIMKEGRPYCCHCFESLYAEYCDTCAQHIG

SEQ ID NO: 482 - protein, 80 aa, Homo sapiens
IIGGDRPDEFLQVKNVLKDGPAAQDGKIAPGDVIVDINGNCVLGHTHADVVQMFQLVPVNQYVNLTLCRGYPLPDDSEDP

SEQ ID NO: 483 - protein, 80 aa, Homo sapiens
CCPECDTRVTSQCLDQNGHKLYRSGDNWTHSCQQCRCLEGEVDCWPLTCPNLSCEYTAILEGECCPRCVSDPCLADNITY

SEQ ID NO: 484 - protein, 80 aa, Homo sapiens
LNSEEKKKRKQRRNRTTFNSSQLQALERVFERTHYPDAFVREDLARRVNLTEARVQVWFQNRRAKFRRNERAMLANKNAS

SEQ ID NO: 485 - protein, 80 aa, Homo sapiens
GLNGGYQDELVDHRLTEREWADEWKHLDHALNCIMEMVEKTRRSMAVLRRCQESDREELNYWKRRYNENTELRKTGTELV

SEQ ID NO: 486 - protein, 80 aa, Homo sapiens
GPGEEAPKKKHRRNRTTFTTYQLHQLERAFEASHYPDVYSREELAAKVHLPEVRVQVWFQNRRAKWRRQERLESGSGAVA

SEQ ID NO: 487 - protein, 80 aa, Homo sapiens
VRMVNGKPKKVRKPRTIYSSYQLAALQRRFQKAQYLALPERAELAAQLGLTQTQVKIWFQNRRSKFKKLYKNGEVPLEHS

SEQ ID NO: 488 - protein, 80 aa, Homo sapiens
EVRFNGKGKKIRKPRTIYSSLQLQALNRRFQQTQYLALPERAELAASLGLTQTQVKIWFQNKRSKFKKLMKQGGAALEGS

SEQ ID NO: 489 - protein, 80 aa, Homo sapiens
GRSEQPKARQRRKPRVLFSQAQVLALERRFKQQRYLSAPEREHLASALQLTSTQVKIWFQNRRYKCKRQRQDKSLELAGH

SEQ ID NO: 490 - protein, 80 aa, Homo sapiens
LPRTLGELQLYRILQKANLLSYFDAFIQQGGDDVQQLCEAGEEEFLEIMALVGMASKPLHVRRLQKALRDWVTNPGLFNQ

SEQ ID NO: 491 - protein, 80 aa, Homo sapiens
NLSLDEDIQKWTVDDVHSFIRSLPGCSDYAQVFKDHAIDGETLPLLTEEHLRGTMGLKLGPALKIQSQVSQHVGSMFYKK

SEQ ID NO: 492 - protein, 80 aa, Homo sapiens
SPEDGSLKKKQRRQRTHFTSQQLQELEATFQRNRYPDMSTREEIAVWTNLTEARVRVWFKNRRAKWRKRERSQQAELCKG

SEQ ID NO: 493 - protein, 80 aa, Homo sapiens
SNLLVSASDDKTLKIWDVSSGKCLKTLKGHSNYVFCCNFNPQSNLIVSGSFDESVRIWDVKTGKCLKTLPAHSDPVSAVH

SEQ ID NO: 494 - protein, 80 aa, Homo sapiens
GNYKSEVNSKPRKERTAFTKEQIRELEAEFAHHNYLTRLRRYEIAVNLDLTERQVKVWFQNRRMKWKRVKGGQQGAAARE

SEQ ID NO: 495 - protein, 80 aa, Homo sapiens
LPRTLGELQLYRVLQRANLLSYYETFIQQGGDDVQQLCEAGEEEFLEIMALVGMATKPLHVRRLQKALREWATNPGLFSQ

SEQ ID NO: 496 - protein, 80 aa, Homo sapiens
PEEPTIGDIYNGKVTSIMQFGCFVQLEGLRKRWEGLVHISELRREGRVANVADVVSKGQRVKVKVLSFTGTKTSLSMKDV

SEQ ID NO: 497 - protein, 80 aa, Homo sapiens
YAFNHPFSINNLMSSEQQHHHSHHHHQPHKMDLKAYEQVMHYPGYGSPMPGSLAMGPVTNKTGLDASPLAADTSYYQGVY

SEQ ID NO: 498 - protein, 80 aa, Homo sapiens
TAAAGPAPPTAPEPAGASSEPEAGDWRPEMSPCSNVVVTDVTSNLLTVTIKEFCNPEDFEKVAAGVAGAAGGGGSIGASK

SEQ ID NO: 499 - protein, 80 aa, Homo sapiens
FLLHNALARKPKRIRTAFSPSQLLRLEHAFEKNHYVVGAERKQLAHSLSLTETQVKVWFQNRRTKFKRQKLEEEGSDSQQ

SEQ ID NO: 500 - protein, 80 aa, Homo sapiens
KRIALYIGNLTWWTTDEDLTEAVHSLGVNDILEIKFFENRANGQSKGFALVGVGSEASSKKLMDLLPKRELHGQNPVVTP

SEQ ID NO: 501 - protein, 80 aa, Homo sapiens
SGAPWYPINSRSRKKRKPYSKLQLAELEGEFLVNEFITRQRRRELSDRLNLSDQQVKIWFQNRRMKKKRLLLREQALSFF

SEQ ID NO: 502 - protein, 80 aa, Homo sapiens
SDNLYPESITSRDCVQLGPPSEGELVELRWTDGNLYKAKFISSVTSHIYQVEFEDGSQLTVKRGDIFTLEEELPKRVRSR

SEQ ID NO: 503 - protein, 80 aa, Homo sapiens
GIPASKVSKWSTDEVSEFIQSLPGCEEHGKVFKDEQIDGEAFLLMTQTDIVKIMSIKLGPALKIFNSILMFKAAEKNSHN

SEQ ID NO: 504 - protein, 80 aa, Homo sapiens
EPSGLHEKRKQRRIRTTFTSAQLKELERVFAETHYPDIYTREELALKIDLTEARVQVWFQNRRAKFRKQERAASAKGAAG

SEQ ID NO: 505 - protein, 80 aa, Homo sapiens
LLLHGPFARKPKRIRTAFSPSQLLRLERAFEKNHYVVGAERKQLAGSLSLSETQVKVWFQNRRTKYKRQKLEEEGPESEQ

SEQ ID NO: 506 - protein, 80 aa, Homo sapiens
SSGNDDDLTIPRAAINKMIKETLPNVRVANDARELVVNCCTEFIHLISSEANEICNKSEKKTISPEHVIQALESLGFGSY

SEQ ID NO: 507 - protein, 80 aa, Homo sapiens
ERRPQAPAKKLRKPRTIYSSLQLQHLNQRFQHTQYLALPERAQLAAQLGLTQTQVKIWFQNKRSKYKKLLKQNSGGQEGD

SEQ ID NO: 508 - protein, 80 aa, Homo sapiens
NVQDRVKRPMNAFIVWSRDQRRKMALENPRMRNSEISKQLGYQWKMLTEAEKWPFFQEAQKLQAMHREKYPNYKYRPRRK

SEQ ID NO: 509 - protein, 80 aa, Homo sapiens
EITRLAVWAAVQAVERKLEAQAMRLLTLEGRTGTNEKKIADCEKTAVEFANHLESKWVVLGTLLQEYGLLQRRLENMENL

SEQ ID NO: 510 - protein, 80 aa, Homo sapiens
CEKDIDECSEGIIECHNHSRCVNLPGWYHCECRSGFHDDGTYSLSGESCIDIDECALRTHTCWNDSACINLAGGFDCLCP

SEQ ID NO: 511 - protein, 80 aa, Homo sapiens
AAISLWTVVAAVQAIERKVEIHSRRLLHLEGRTGTAEKKLASCEKTVTELGNQLEGKWAVLGTLLQEYGLLQRRLENLEN

SEQ ID NO: 512 - protein, 80 aa, Homo sapiens
GQNRPLIKPKRRLSAARRAGTSCANCQTTTTTLWRRNANGDPVCNACGLYYKLHNINRPLTMKKEGIQTRNRKMSSKSKK

SEQ ID NO: 513 - protein, 80 aa, Homo sapiens
HAELPGKHCRRRKARTVFSDSQLSGLEKRFEIQRYLSTPERVELATALSLSETQVKTWFQNRRMKHKKQLRKSQDEPKAP

SEQ ID NO: 514 - protein, 80 aa, Homo sapiens
QDATVYVGGLDEKVSEPLLWELFLQAGPVVNTHMPKDRVTGQHQGYGFVEFLSEEDADYAIKIMNMIKLYGKPIRVNKAS

SEQ ID NO: 515 - protein, 80 aa, Homo sapiens
PIDNDAEGVWSPDIEQSFQEALAIYPPCGRRKIILSDEGKMYGRNELIARYIKLRTGKTRTRKQVSSHIQVLARRKSRDF

SEQ ID NO: 516 - protein, 80 aa, Homo sapiens
GLDNDAEGVWSPDIEQSFQEALAIYPPCGRRKIILSDEGKMYGRNELIARYIKLRTGKTRTRKQVSSHIQVLARKKVREY

SEQ ID NO: 517 - protein, 80 aa, Homo sapiens
DSVGTPQSNGGMRLHDFVSKTVIKPESCVPCGKRIKFGKLSLKCRDCRVVSHPECRDRCPLPCIPTLIGTPVKIGEGMLA

SEQ ID NO: 518 - protein, 80 aa, Homo sapiens
SAPHSMTASSSSVSSPSPGLPRRSAPPSPLCRSLSPGTGGGVRGGVGYLSRGDPVRVLARRVRPDGSVQYLVEWGGGGIF

SEQ ID NO: 519 - protein, 80 aa, Homo sapiens
GDPHYSFNHPFSINNLMSSSEQQHKLDFKAYEQALQYSPYGSTLPASLPLGSASVTTRSPIEPSALEPAYYQGVYSRPVL

SEQ ID NO: 520 - protein, 80 aa, Homo sapiens
GQNRPLIKPKRRLSAARRAGTCCANCQTTTTTLWRRNANGDPVCNACGLYYKLHNVNRPLTMKKEGIQTRNRKMSNKSKK

SEQ ID NO: 521 - protein, 80 aa, Homo sapiens
DSLSGSSLYSTSANLPVMGHEKFPSDLDLDMFNGSLECDMESIIRSELMDADGLDFNFDSLISTQNVVGLNVGNFTGAKQ

SEQ ID NO: 522 - protein, 80 aa, Homo sapiens
TEISLWTVVAAIQAVEKKMESQAARLQSLEGRTGTAEKKLADCEKMAVEFGNQLEGKWAVLGTLLQEYGLLQRRLENVEN

SEQ ID NO: 523 - protein, 80 aa, Homo sapiens
MDSGTRRKNATRETTSTLKAWLQEHRKNPYPTKGEKIMLAIITKMTLTQVSTWFANARRRLKKENKMTWPPRNKCADEKR

SEQ ID NO: 524 - protein, 80 aa, Homo sapiens
NIEKQIYLPSTRAKTSIVWHFFHVDPQYTWRAICNLCEKSVSRGKPGSHLGTSTLQRHLQARHSPHWTRANKFGVASGEE

SEQ ID NO: 525 - protein, 80 aa, Homo sapiens
AKQNDDSEAGAKRPRTTITAKQLETLKNAYKNSPKPARHVREQLSSETGLDMRVVQVWFQNRRAKEKRLKKDAGRHRWGQ

SEQ ID NO: 526 - protein, 80 aa, Homo sapiens
DALSYLDQVKLQFGSQPQVYNDFLDIMKEFKSQSIDTPGVISRVSQLFKGHPDLIMGFNTFLPPGYKIEVQTNDMVNVTT

SEQ ID NO: 527 - protein, 80 aa, Homo sapiens
DDHTVCLWDINAGPKEGKIVDAKAIFTGHSAVVEDVAWHLLHESLFGSVADDQKLMIWDTRSNTTSKPSHLVDAHTAEVN

SEQ ID NO: 528 - protein, 80 aa, Homo sapiens
GSILLDKDGKRKHTRPTFSGQQIFALEKTFEQTKYLAGPERARLAYSLGMTESQVKVWFQNRRTKWRKKHAAEMATAKKK

SEQ ID NO: 529 - protein, 80 aa, Homo sapiens
DPTALVEAIVEEVACPICMTFLREPMSIDCGHSFCHSCLSGLWEIPGESQNWGYTCPLCRAPVQPRNLRPNWQLANVVEK

SEQ ID NO: 530 - protein, 80 aa, Homo sapiens
QSLPKKVSLSSDTTRKPLEIRSPSAESKKPKWVPPAASGGSRSSSSPLVVVSVKSPNQSLRLGLSRLARVKPLHPNATST

SEQ ID NO: 531 - protein, 80 aa, Homo sapiens
AKSSQRKQRDCVNQCKSKPGLSTSIPLRMSSYTFKRPVTRITPHPGNEVRYHQWEESLEKPQQVCWQRRLQGLQAYSSAG

SEQ ID NO: 532 - protein, 80 aa, Homo sapiens
VRMVNGKPKKVRKPRTIYSSFQLAALQRRFQKTQYLALPERAELAASLGLTQTQVKIWFQNKRSKIKKIMKNGEMPPEHS

SEQ ID NO: 533 - protein, 80 aa, Homo sapiens
LQCNNHACGWDGGDCSLNFNDPWKNCTQSLQCWKYFSDGHCDSQCNSAGCLFDGFDCQRAEGQCNPLYDQYCKDHFSDGH

SEQ ID NO: 534 - protein, 80 aa, Homo sapiens
ETWVEEDELFQVQAAPDEDSTTNITKKQKWTVEESEWVKAGVQKYGEGNWAAISKNYPFVNRTAVMIKDRWRTMKRLGMN

SEQ ID NO: 535 - protein, 80 aa, Homo sapiens
AEISLWTVVAAIQAVERKVDAQASQLLNLEGRTGTAEKKLADCEKTAVEFGNHMESKWAVLGTLLQEYGLLQRRLENLEN

SEQ ID NO: 536 - protein, 80 aa, Homo sapiens
LEKRTLFRLDLVPINRSVGLKAKPTKPVTEVLRPVVARYGLDLSGLLVRLSGEKEPLDLGAPISSLDGQRVVLEEKDPSR

SEQ ID NO: 537 - protein, 80 aa, Homo sapiens
PNCLSSSMQLPHGGGRHQELVRFRDVAVVFSPEEWDHLTPEQRNLYKDVMLDNCKYLASLGNWTYKAHVMSSLKQGKEPW

SEQ ID NO: 538 - protein, 80 aa, Homo sapiens
DDYKEGDLRIMPESSESPPTEREPGGVVDGLIGKHVEYTKEDGSKRIGMVIHQVEAKPSVYFIKFDDDFHIYVYDLVKKS

SEQ ID NO: 539 - protein, 80 aa, Homo sapiens
SEPDLPLKRKQRRSRTTFTAEQLEELEKAFERTHYPDIYTREELAQRTKLTEARVQVWFSNRRARWRKQAGANQLAAFNH

SEQ ID NO: 540 - protein, 80 aa, Homo sapiens
AGGVLDKDGKKKHSRPTFSGQQIFALEKTFEQTKYLAGPERARLAYSLGMTESQVKVWFQNRRTKWRKRHAVEMASAKKK

SEQ ID NO: 541 - protein, 80 aa, Homo sapiens
DVMSFSVTVTTIPASQAMNPSSHGQTIPVQAFSEENSIEGTPSKCYCRLKAMIMCKGCGAFCHDDCIGPSKLCVSCLVVR

SEQ ID NO: 542 - protein, 80 aa, Homo sapiens
GGYSSVSSCNGYGRMGLLHQEKLPSDLDGMFIERLDCDMESIIRNDLMDGDTLDFNFDNVLPNQSFPHSVKTTTHSWVSG

SEQ ID NO: 543 - protein, 80 aa, Homo sapiens
GGSPTGFGCKSRPKARSSTGRECVNCGATSTPLWRRDGTGHYLCNACGLYHKMNGQNRPLIKPKRRLSAARRAGTSCANC

SEQ ID NO: 544 - protein, 80 aa, Homo sapiens
GQNRPLIRPKKRLIVSKRAGTQCTNCQTTTTTLWRRNASGDPVCNACGLYYKLHQVNRPLTMRKDGIQTRNRKASGKGKK

SEQ ID NO: 545 - protein, 80 aa, Homo sapiens
PVALLRKQNFQPTAQQQLTKPAKITCANCKKPLQKGQTAYQRKGSAHLFCSTTCLSSFSHKRTQNTRSIICKKDASTKKA

SEQ ID NO: 546 - protein, 80 aa, Homo sapiens
TEITLWTVVAAIQALEKKVDSCLTRLLTLEGRTGTAEKKLADCEKTAVEFGNQLEGKWAVLGTLLQEYGLLQRRLENVEN

SEQ ID NO: 547 - protein, 80 aa, Homo sapiens
KKQRGRPSSQPRRNIVGCRISHGWKEGDEPITQWKGTVLDQVPINPSLYLVKYDGIDCVYGLELHRDERVLSLKILSDRV

SEQ ID NO: 548 - protein, 80 aa, Homo sapiens
WTCDLDDDCGDRSDESASCAYPTCFPLTQFTCNNGRCININWRCDNDNDCGDNSDEAGCSHSCSSTQFKCNSGRCIPEHW

SEQ ID NO: 549 - protein, 80 aa, Homo sapiens
PKGAAAPSASQRRKRTSFSAEQLQLLELVFRRTRYPDIHLRERLAALTLLPESRIQVWFQNRRAKSRRQSGKSFQPLARP

SEQ ID NO: 550 - protein, 80 aa, Homo sapiens
KIKYDWYQTESQVVITLMIKNVQKNDVNVEFSEKELSALVKLPSGEDYNLKLELLHPIIPEQSTFKVLSTKIEIKLKKPE

SEQ ID NO: 551 - protein, 80 aa, Homo sapiens
DPSKEVEYVCELCKGAAPPDSPVVYSDRAGYNKQWHPTCFVCAKCSEPLVDLIYFWKDGAPWCGRHYCESLRPRCSGCDE

SEQ ID NO: 552 - protein, 80 aa, Homo sapiens
GSGAGKAKKSVDKNSNEYRVRRERNNIAVRKSRDKAKQRNVETQQKVLELTSDNDRLRKRVEQLSRELDTLRGIFRQLPE

SEQ ID NO: 553 - protein, 80 aa, Homo sapiens
GPASSFTPKQRSKARSCSEGRECVNCGATATPLWRRDGTGHYLCNACGLYHKMNGQNRPLIKPKRRLSAARRAGTCCANC

SEQ ID NO: 554 - protein, 80 aa, Homo sapiens
KPSDHIKRPMNAFMVWSRGQRRKMAQENPKMHNSEISKRLGAEWKLLSEAEKRPYIDEAKRLRAQHMKEHPDYKYRPRRK

SEQ ID NO: 555 - protein, 80 aa, Homo sapiens
LYSGFQQTADKCSVCGHLIMEMILQALGKSYHPGCFRCSVCNECLDGVPFTVDVENNIYCVRDYHTVFAPKCASCARPIL

SEQ ID NO: 556 - protein, 80 aa, Homo sapiens
HPSQDLVFSASPDATIRIWSVPNASCVQVVRAHESAVTGLSLHATGDYLLSSSDDQYWAFSDIQTGRVLTKVTDETSGCS

SEQ ID NO: 557 - protein, 80 aa, Homo sapiens
ELSAVGERVFAAESIIKRRIRKGRIEYLVKWKGWAIKYSTWEPEENILDSRLIAAFEQKERERELYGPKKRGPKPKTFLL

SEQ ID NO: 558 - protein, 80 aa, Homo sapiens
RTGSDSKSGKPRRARTAFTYEQLVALENKFKATRYLSVCERLNLALSLSLTETQVKIWFQNRRTKWKKQNPGADTSAPTG

SEQ ID NO: 559 - protein, 80 aa, Homo sapiens
VWDLSKIGEEQSPEDAEDGPPELLFIHGGHTAKISDFSWNPNEPWVICSVSEDNIMQVWQMAENIYNDEDPEGSVDPEGQ

SEQ ID NO: 560 - protein, 80 aa, Homo sapiens
ERCTPAGGGAEPRKLSRTPKCARCRNHGVVSCLKGHKRFCRWRDCQCANCLLVVERQRVMAAQVALRRQQATEDKKGLSG

SEQ ID NO: 561 - protein, 80 aa, Homo sapiens
SQPGALIPGDPQAMSQPNRGPSPFSPVQLHQLRAQILAYKMLARGQPLPETLQLAVQGKRTLPGLQQQQQQQQQQQQQQQ

SEQ ID NO: 562 - protein, 573 aa, Homo sapiens; identical to SEQ ID NO: 89

SEQ ID NO: 563 - protein, 441 aa, Homo sapiens
MSEREVSTAPAGTDMPAAKKQKLSSDENSNPDLSGDENDDAVSIESGTNTERPDTPTNTPNAPGRKSWGKGKWKSKKCKYSFKCVNSLKEDHNQPLFGVQFNWHSKEGDPLVFATVGSNRVTLYECHSQGEIRLLQSYVDADADENFYTCAWTYDSNTSHPLLAVAGSRGIIRIINPITMQCIKHYVGHGNAINELKFHPRDPNLLLSVSKDHALRLWNIQTDTLVAIFGGVEGHRDEVLSADYDLLGEKIMSCGMDHSLKLWRINSKRMMNAIKESYDYNPNKTNRPFISQKIHFPDFSTRDIHRNYVDCVRWLGDLILSKSCENAIVCWKPGKMEDDIDKIKPSESNVTILGRFDYSQCDIWYMRFSMDFWQKMLALGNQVGKLYVWDLEVEDPHKAKCTTLTHHKCGAAIRQTSFSRDSSILIAVCDDASIWRWDRLR

SEQ ID NO: 564 - protein, 485 aa, Homo sapiens
MPAMVEKGPEVSGKRRGRNNAAASASAAAASAAASAACASPAATAASGAAASSASAAAASAAAAPNNGQNKSLAAAAPNGNSSSNSWEEGSSGSSSDEEHGGGGMRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARKQKREREESEDELEEANGNNPIDIEVDQNKESKKEVPPTETVPQVKKEKHSTQAKNRAKRKPPKGMFLSQEDVEAVSANATAATTVLRQLDMELVSVKRQIQNIKQTNSALKEKLDGGIEPYRLPEVIQKCNARWTTEEQLLAVQAIRKYGRDFQAISDVIGNKSVVQVKNFFVNYRRRFNIDEVLQEWEAEHGKEETNGPSNQKPVKSPDNSIKMPEEEDEAPVLDVRYASAS

SEQ ID NO: 565 - protein, 77 aa, Homo sapiens
TGRTLVTFKDVFVDFTREEWKLLDTAQQIVYRNVMLENYKNLVSLGYQLTKPDVILRLEKGEEPLEINLWITKFVKD

SEQ ID NO: 566 - protein, 120 aa, Homo sapiens
MYPYDVPDYASPKKKRKVGGGASMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 567 - protein, 117 aa, Homo sapiens
ALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 568 - protein, 62 aa, Homo sapiens
RTLVTFKDVFVDFTREEWKLLDTAQQIVYRNVMLENYKNLVSLGYQLTKPDVILRLEKGEEP

SEQ ID NO: 569 - protein, 98 aa, Homo sapiens
RTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQS

SEQ ID NO: 570 - protein, 99 aa, synthetic construct
RTLVTFKDVAVDFTQEEWQQLDPAQKIVYRDVMLENYSNLVSVGYQLTKPDVILRLEQKGEEPWLVEEEIHQETHPDSETAFEIKSSVSSRSIFKDKQS

SEQ ID NO: 571 - protein, 95 aa, synthetic construct
RTLVTFKDVFVDFTREEWKLLDTAQQIVYRNVMLENYKNLVSLGYQLTKPDVILRLEKGEEPWLEEEEVLGSGRAEKNGDIGGQIWKPKDVKESL

SEQ ID NO: 572 - protein, 103 aa, synthetic construct
MNNSQGRVTFEDVTVNFTQGEWQRLNPEQRNLYRDVMLENYSNLVSVGQGETTKPDVILRLEQGKEPWLVEREIHQETHPDSETAFEIKSSVSSRSIFKDKQS

SEQ ID NO: 573 - protein, 1615 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKPEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 574 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 575 - protein, 301 aa, Homo sapiens
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 576 - protein, 853 aa, Homo sapiens
MKGDTRHLNGEEDAGGREDSILVNGACSDQSSDSPPILEAIRTPEIRGRRSSSRLSKREVSSLLSYTQDLTGDGDGEDGDGSDTPVMPKLFRETRTRSESPAVRTRNNNSVSSRERHRPSPRSTRGRQGRNHVDESPVEFPATRSLRRRATASAGTPWPSPPSSYLTIDLTDDTEDTHGTPQSSSTPYARLAQDSQQGGMESPQVEADSGDGDSSEYQDGKEFGIGDLVWGKIKGFSWWPAMVVSWKATSKRQAMSGMRWVQWFGDGKFSEVSADKLVALGLFSQHFNLATFNKLVSYRKAMYHALEKARVRAGKTFPSSPGDSLEDQLKPMLEWAHGGFKPTGIEGLKPNNTQPVVNKSKVRRAGSRKLESRKYENKTRRRTADDSATSDYCPAPKRLKTNCYNNGKDRGDEDQSREQMASDVANNKSSLEDGCLSCGRKNPVSFHPLFEGGLCQTCRDRFLELFYMYDDDGYQSYCTVCCEGRELLLCSNTSCCRCFCVECLEVLVGTGTAAEAKLQEPWSCYMCLPQRCHGVLRRRKDWNVRLQAFFTSDTGLEYEAPKLYPAIPAARRRPIRVLSLFDGIATGYLVLKELGIKVGKYVASEVCEESIAVGTVKHEGNIKYVNDVRNITKKNIEEWGPFDLVIGGSPCNDLSNVNPARKGLYEGTGRLFFEFYHLLNYSRPKEGDDRPFFWMFENVVAMKVGDKRDISRFLECNPVMIDAIKVSAAHRARYFWGNLPGMNRPVIASKNDKLELQDCLEYNRIAKLKKVQTITTKSNSIKQGKNQLFPVVMNGKEDVLWCTELERIFGFPVHYTDVSNMGRGARQKLLGRSWSVPVIRHLFAPLKDYFACE

SEQ ID NO: 577 - protein, 740 aa, Homo sapiens
MRGGSRHLSNEEDVSGCEDCIIISGTCSDQSSDPKTVPLTQVLEAVCTVENRGCRTSSQPSKRKASSLISYVQDLTGDGDEDRDGEVGGSSGSGTPVMPQLFCETRIPSKTPAPLSWQANTSASTPWLSPASPYPIIDLTDEDVIPQSISTPSVDWSQDSHQEGMDTTQVDAESRDGGNIEYQVSADKLLLSQSCILAAFYKLVPYRESIYRTLEKARVRAGKACPSSPGESLEDQLKPMLEWAHGGFKPTGIEGLKPNKKQPENKSRRRTTNDPAASESSPPKRLKTNSYGGKDRGEDEESREQMASDVTNNKGNLEDHCLSCGRKDPVSFHPLFEGGLCQSCRDRFLELFYMYDEDGYQSYCTVCCEGRELLLCSNTSCCRCFCVECLEVLVGAGTAEDVKLQEPWSCYMCLPQRCHGVLRRRKDWNMRLQDFFTTDPDLEEFEPPKLYPAIPAAKRRPIRVLSLFDGIATGYLVLKELGIKVEKYIASEVCAESIAVGTVKHEGQIKYVDDIRNITKEHIDEWGPFDLVIGGSPCNDLSCVNPVRKGLFEGTGRLFFEFYRLLNYSCPEEEDDRPFFWMFENVVAMEVGDKRDISRFLECNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPEHYTDVSNMGRGARQKLLGRSWSVPVIRHLFAPLKDHFACE

SEQ ID NO: 578 - protein, 387 aa, Homo sapiens
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSSRHWALVSEEELSLLAQNKQSSKLAAKWPTKLVKNCFLPLREYFKYFSTELTSSL

SEQ ID NO: 579 - protein, 213 aa, Homo sapiens
NPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 580 - protein, 421 aa, Mus musculus
MGSRETPSSCSKTLETLDLETSDSSSPDADSPLEEQWLKSSPALKEDSVDVVLEDCKEPLSPSSPPTGREMIRYEVKVNRRSIEDICLCCGTLQVYTRHPLFEGGLCAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 581 - protein, 214 aa, Mus musculus
GPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 582 - protein, 403 aa, Ailuropoda melanoleuca
MALSPTGTLSVETLDRSDPDPLDEGPWQATCEILLEPDAEHSTDVILVGSSELSAPASPGPRRDLLAYEVKVNQRDIEDVCICCGSLRVHTQHPLFEGGMCAPCKDKFLDCLFLYDDDGYQSYCSICCAGETLLICENPDCTRPSLMMKLRLFRECACLIFPSEGMLLQTVWFWKMTVVWQPGLRHLPQENPLETYKTVPVWKREPVRVLSLFGDIRRELMSLGFLESGSAPGRLKHLDDVTDVVRKDVEGWGPFDLVYGSTPPIGHACDHPPVWYLLQFHRILQYARPRPGSQQPFFWMFVDNLVLSQDDQTAATRFLEADPVTIQDVCGRAVRNTVHVWSNIPAVRSRHSALALCEELSLLAQDRQRTKPPAQGPAQLVKNCFLPLREYFKYFSTELTSSL

SEQ ID NO: 583 - protein, 213 aa, Ailuropoda melanoleuca
NPLETYKTVPVWKREPVRVLSLFGDIRRELMSLGFLESGSAPGRLKHLDDVTDVVRKDVEGWGPFDLVYGSTPPIGHACDHPPVWYLLQFHRILQYARPRPGSQQPFFWMFVDNLVLSQDDQTAATRFLEADPVTIQDVCGRAVRNTVHVWSNIPAVRSRHSALALCEELSLLAQDRQRTKPPAQGPAQLVKNCFLPLREYFKYFSTELTSSL

SEQ ID NO: 584 - protein, 426 aa, Carlito syrichta
MALSCRRTLPLESLHSSNSDLASQLDKEQWRPPCETHGIPVAAAPVLDLEAECSLDVILVGSSELSTSSSPRLGRDHIAYEVKVNQRNIEDICLCCGSFLVHTQHPLFEGGMCAPCKDKFLDTLFLYDEDGYQSYCSICCSGETLLICENPDCTRCYCFECLDTLVSPGTSEKVHAMSNWVCFLCLPFTRSGLLQRRRKWRGQLKAFYDRESESSLEMYKTVPVWKREPVRVLSLFGDIKKELMSLGFVETGSDPGRLRHLDDTTNIVRRNVEEWGPFHLLYGATPPLGHTCDRPPGWYLFQFHRLLQYARPQPGSPQPFFWMFVDNVMLTREDRAIASRFLETEPVTIPDIHGRALQNAVCVWSNIPAVRSKHSALVSEEELSLLAQDRQRAKLPTQGPTKLVKNCFLPLREYFKYFSTELTSFL

SEQ ID NO: 585 - protein, 213 aa, Carlito syrichta
SSLEMYKTVPVWKREPVRVLSLFGDIKKELMSLGFVETGSDPGRLRHLDDTTNIVRRNVEEWGPFHLLYGATPPLGHTCDRPPGWYLFQFHRLLQYARPQPGSPQPFFWMFVDNVMLTREDRAIASRFLETEPVTIPDIHGRALQNAVCVWSNIPAVRSKHSALVSEEELSLLAQDRQRAKLPTQGPTKLVKNCFLPLREYFKYFSTELTSFL

SEQ ID NO: 586 - protein, 424 aa, Meriones unguiculatus
MGSQETPSTRAKTPGTWNLESTDSSSPESLGHLEEQWANSSPDLKDEHSKDVEPEDSKELISSASPPSGREIIRYEISVNQRNIEDICLCCGTLQVYKQHPLFEGGICAPCKDKFLETFFLYDEDGHQSYCSICCSGGTLFICESPDCTRCYCFECVDILVGPGTSERINAMPCWVCFLCLPFTRSGLLQRRRKWRHQLKAFFDEGGASPLEMYKTVSAWKRKPMRVLSLFKNIDKELKNLGFLESGSGSEEERLKYLEDVTNVVRRDVEKWGPFDLVYGSTRPRGSSCDHCPAWYMFQFHRILQYARPPSGSEQPFFWVFVDNLLMTEDDQITADRFLQMKAVTLQDVRGRVLQNAVRVWSNIPGVKSKHMALTEKEEQSLEAQAGTRTKLSAQKVDPLVKNCLLPLREYFKFFSQNSLPLDK

SEQ ID NO: 587 - protein, 216 aa, Meriones unguiculatus
SPLEMYKTVSAWKRKPMRVLSLFKNIDKELKNLGFLESGSGSEEERLKYLEDVTNVVRRDVEKWGPFDLVYGSTRPRGSSCDHCPAWYMFQFHRILQYARPPSGSEQPFFWVFVDNLLMTEDDQITADRFLQMKAVTLQDVRGRVLQNAVRVWSNIPGVKSKHMALTEKEEQSLEAQAGTRTKLSAQKVDPLVKNCLLPLREYFKFFSQNSLPLDK

SEQ ID NO: 588 - protein, 409 aa, Ochotona princeps
MALPSPETLDSLDRVPASHPDEQHWTVCDNSDPILEVEAEGSMDVILVDDSPAPSGRDRIELEVKVNQRSIEDLCLCCGSSQVHRQHPLFQGGLCAPCKDKFLEALFLYDEDGYQSYCSICGLGDTLLVCESPDCTRGYCFACVDGLVGAGSSGHMHTVSPWVCFLCVPGSRHGLLQRRRRWRTQLKVFHEQEAAQPLEIYETVPACRRKPLRVLSLFEHIEKELASLGFLETGSSPGRIRHLDDVTDVVRRDVEQWGPFDLVYGSTPPLGHASPRSPGWYLFQFHRMLQYTQPTASTQRPFFWMFVDNLLLTRDDLVTATRFLEVEPATLQDVRGRVLQGAMRVWSNIPAVNSRHTELAPEAETALLAQSCRRAKASGEGLARLLKSCFLPLREYFKYFPQSPLPLRK

SEQ ID NO: 589 - protein, 214 aa, Ochotona princeps
QPLEIYETVPACRRKPLRVLSLFEHIEKELASLGFLETGSSPGRIRHLDDVTDVVRRDVEQWGPFDLVYGSTPPLGHASPRSPGWYLFQFHRMLQYTQPTASTQRPFFWMFVDNLLLTRDDLVTATRFLEVEPATLQDVRGRVLQGAMRVWSNIPAVNSRHTELAPEAETALLAQSCRRAKASGEGLARLLKSCFLPLREYFKYFPQSPLPLRK

SEQ ID NO: 590 - protein, 224 aa, Neosciurus carolinensis
MGGPRPAAVEESPHEIYKTVPAWKREPMRVLSLFGDIGKELTSLGFLETGSEAGRLKHLEDVTDTVRRDVEEWGPFDLVYGSTPALGHSCDRSPGWYLFQFHRLLQYARPRLGSPKPFFWMFVDNLLLTKDDQAIASRFLEMEPVTLQDVHGRVLQNAVRVWTNVPAVKSRHSALASEEELLLVQDGQRGRLPAQGPAALVKHCFLPLREYFKYFSQNTLPLYK

SEQ ID NO: 591 - protein, 213 aa, Neosciurus carolinensis
SPHEIYKTVPAWKREPMRVLSLFGDIGKELTSLGFLETGSEAGRLKHLEDVTDTVRRDVEEWGPFDLVYGSTPALGHSCDRSPGWYLFQFHRLLQYARPRLGSPKPFFWMFVDNLLLTKDDQAIASRFLEMEPVTLQDVHGRVLQNAVRVWTNVPAVKSRHSALASEEELLLVQDGQRGRLPAQGPAALVKHCFLPLREYFKYFSQNTLPLYK

SEQ ID NO: 592 - protein, 417 aa, Bison bison
MARSSPGTLNLEIMDGSDPDPALPPDREQWPPPCEILLDPEPEHSLDIILVGSSELSSPPSPGPRRDFIAYEVKVNQRDIEDVCICCGSLQLHTQHPLFEGGMCAPCKDKFLECLFLYDDDGYQSYCSICCAGETLLICENPDCTRCYCFECVDTLVGPGTSGKVHAMSNWVCFLCLPFPRSGLLQRRRKWRTWLKAFYDREAESPLVMYKTVPVWKREPIRVLSLFGDIKKELTSLGFLEDGSKPGRLKHLDDVTNIVRRDIDEWGPFDLTYGSTPTLGHTCDHPPGWYVYQFHRILQYARPLPGSPQPFFWMFVDNLVLTEEDLDVATRFLETDPVTIQDVRGRTVQNAVHVWSNIPAVKSRHSALVSQEELSLLAQDRQRVKSPVQGPATLVKNCFLPLREYFKYFSTELTSSL

SEQ ID NO: 593 - protein, 213 aa, Bison bison
SPLVMYKTVPVWKREPIRVLSLFGDIKKELTSLGFLEDGSKPGRLKHLDDVTNIVRRDIDEWGPFDLTYGSTPTLGHTCDHPPGWYVYQFHRILQYARPLPGSPQPFFWMFVDNLVLTEEDLDVATRFLETDPVTIQDVRGRTVQNAVHVWSNIPAVKSRHSALVSQEELSLLAQDRQRVKSPVQGPATLVKNCFLPLREYFKYFSTELTSSL

SEQ ID NO: 594 - protein, 443 aa, Equus przewalskii
MALSSPGTLSLETLDSWDPDVAGQLDEERWQPSSEIVGRPMAAAPVLDLEEEPSMDIILVDSSELSSPPSPGPSRDMCICCGSFQVHTQHPLFEGGMCAACKDKFLSCLFLYDDDGNQSYCSICCSGETLLICENPDCTRCYCFECVDTLVSPRTSEKVQAMSNWVCFLCLPFPRSGLLQRRRKWRGWLKAFYDQEAVRSRSAWGRRMRSGPHLVGFLWLLVAKCPSALESPLEMYKTVPVWKREPVRVLSLFGDIKKELTTLGFLENGSDPGRLKHLDDVTNTVRRDVEEWGPFDLVYGSTPPLGHACDHPPGWYLFQFHRVLQYARPRPGSPQAFFWMFVDNLVLTEDDRAVATRFLETDPVTIQDVCGRAVRNAVHVWSNIPAVKSRHSALFSQEESFLRAQDRQRAKPPARGPAKLVKNCFLPLREYFKYFSTEFTSSL

SEQ ID NO: 595 - protein, 213 aa, Equus przewalskii
SPLEMYKTVPVWKREPVRVLSLFGDIKKELTTLGFLENGSDPGRLKHLDDVTNTVRRDVEEWGPFDLVYGSTPPLGHACDHPPGWYLFQFHRVLQYARPRPGSPQAFFWMFVDNLVLTEDDRAVATRFLETDPVTIQDVCGRAVRNAVHVWSNIPAVKSRHSALFSQEESFLRAQDRQRAKPPARGPAKLVKNCFLPLREYFKYFSTEFTSSL

SEQ ID NO: 596 - protein, 415 aa, Mus caroli
MGSRETPSSFSKTLETLDLETSDSSSPDADSPLEEQWLKSSPALKEDNVDMVLEDCKEPLSPSSPPTGREMIRYEVKVNRRSIEDICLCCGTLQVYTQHPLFEGGICAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSTWKRQPVRVLSLFGNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLMTEDDQETTARFLQTEAVTLQDVRGRDYQNVMRVWSNIPGLKSKHVPLTPKEEEYLQAQVRTRSKLDAQKVDLLVKNCLLPLREYFKYFS

SEQ ID NO: 597 - protein, 208 aa, Mus caroli
GPMEIYKTVSTWKRQPVRVLSLFGNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLMTEDDQETTARFLQTEAVTLQDVRGRDYQNVMRVWSNIPGLKSKHVPLTPKEEEYLQAQVRTRSKLDAQKVDLLVKNCLLPLREYFKYFS

SEQ ID NO: 598 - protein, 387 aa, Pan troglodytes
MAAIPALDPEAEPSMDVILVGSSELSSSISPRTGRDLIAYEVKANQRNIEDICICCGSLQVHTQHPLFEGGICAPCKDKSLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSSRHWALVSEEELSLLAQNKQSSKLAAKWPTKLVKNCFLPLREYFKYFSTELTSSL

SEQ ID NO: 599 - protein, 214 aa, Pan troglodytes
NPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSSRHWALVSEEELSLLAQNKQSSKLAAKWPTKLVKNCFLPLREYFKYFSTELTSSL

SEQ ID NO: 600 - protein, 391 aa, Homo sapiens
MEPLRVLELYSGVGGMHHALRESCIPAQVVAAIDVNTVANEVYKYNFPHTQLLAKTIEGITLEEFDRLSFDMILMSPPCQPFTRIGRQGDMTDSRTNSFLHILDILPRLQKLPKYILLENVKGFEVSSTRDLLIQTIENCGFQYQEFLLSPTSLGIPNSRLRYFLIAKLQSEPLPFQAPGQVLMEFPKIESVHPQKYAMDVENKIQEKNVEPNISFDGSIQCSGKDAILFKLETAEEIHRKNQQDSDLSVKMLKDFLEDDTDVNQYLLPPKSLLRYALLLDIVQPTCRRSVCFTKGYGSYIEGTGSVLQTAEDVQVENIYKSLTNLSQEEQITKLLILKLRYFTPKEIANLLGFPPEFGFPEKITVKQRYRLLGNSLNVHVVAKLIKILYE

SEQ ID NO: 601 - protein, 353 aa, unidentified
  source 1..353: note = Mycoplasmatales bacterium
MAEWYIPAIVSYQAIHNGFTLNKINHKIELQTMIDYLESKTLSMNSKEPVKRGFWYKKHLDEIRIVYTAVKMSEQEGNIFDVRTLFERGLSDIDLLTYSFPCQDLSQQGKQKGMGRDSQTRSGLLWEIEKALDTSKKEDLPKYLLMENVVALTHKVNAEELDEWMMKLESLGYKNDLRILNAGDFGSSQARRRTFMISTLNEKVELPVGNKKPKSMNKILNDEPTRKDFLPALDKFDLTEYKWTKSNINKAKLINYSTFNSEAYVYDSNFTGPTLTASGANSRIKFEYNGKIRKIGAEEAYAYMGFKKSDYIKVNKLNYLNETKMIYTCGNSISVEVLRSIMTNINNNFKENK

SEQ ID NO: 602 - protein, 398 aa, Mycoplasma marinum
MLFLIGTFKYVLIYITKVIRIFEAFAGIGAQRKALRNIKSNYEVSGMAEWYIPAIVSYQAIHNGFTLSRVDKKTKLTEMIKYLESKTLSMDSKEPVRTGYWFKKHKDMVRIVYSAVKLSEAEGNIFDVRTLHERKLEDIDLLTYSFPCQDLSQQGKQRGMKKDSGTRSGLLWEIEKALEATPKDKLPKYLLMENVVALTHKTNKKDLDNWKRKLRSLGYYNDINVLNAGDFGSSQARRRAFMISTLDSKVTLPLGDKKPQAISKILNKETRSQDFMPALDEYEKTDFKRTLSNIKKCKLIDYTSFNSEAYVYDPKYTGPTLTASGANSRIKFTHQGKMRKINAEEAYRYMGFSTNDYKKVNNLNFLSETKMIYTCGNSISVEVLEEIMLKIIREDNNG

SEQ ID NO: 603 - protein, 381 aa, Spiroplasma chinense
MKKIRLFEAFAGIGSQRRALKSVVGNNFEIAGLAEWYVPAIVMYQIINNDFSKKNVLDNVPRDEVIDYLNSKCLSWDSKKPVSKNFWNRKSQDILNVIYSAVKKSEEEGNIFDVRTLHERTLESIDILTYSFPCQDLSQQGIQKGMKKNSGTRSGLLWEIEKAIDNTPKNNLPKILLMENVPALLNKTNELELKEWLIKLENMGYKNSIGILNAADFGSPQARRRVFMISSRNKKIELPVGKSKPGKLNDILEKNVEDKFIMTNLEKYDFSEFSLTKSNIKKCSLINYTKFNSEAYVYDPDFTGPTLTASGANSRIKIYDKGFIRRMSPLESFRYMGFDDEDYKKIDEFEFLTDTQKIFVCGNSISIEVLKAIFERIDSNE

SEQ ID NO: 604 - protein, 395 aa, Mycoplasma penetrans
MNSNKDKIKVIKVFEAFAGIGSQFKALKNIARSKNWEIQHSGMVEWFVDAIVSYVAIHSKNFNPKIEQLDKDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRSLKNYTTFNSENYVYNINGIGPTLTASGANSRIKIETQQGVRYLTPLECFKYMQFDVNDFKKVQSTNLISENKMIYIAGNSIPVKILEAIFNTLEFVNNEE

SEQ ID NO: 605 - protein, 386 aa, Spiroplasma monobiae
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 606 - protein, 358 aa, Haemophilus parainfluenzae
MKDVLDDNLLEEPAAQYSLFEPESNPNLREKFTFIDLFAGIGGFRIAMQNLGGKCIFSSEWDEQAQKTYEANFGDLPYGDITLEETKAFIPEKFDILCAGFPCQAFSIAGKRGGFEDTRGTLFFDVAEIIRRHQPKAFFLENVKGLKNHDKGRTLKTILNVLREDLGYFVPEPAIVNAKNFGVPQNRERIYIVGFHKSTGVNSFSYPEPLDKIVTFADIREEKTVPTKYYLSTQYIDTLRKHKERHESKGNGFGYEIIPDDGIANAIVVGGMGRERNLVIDHRITDFTPTTNIKGEVNREGIRKMTPREWARLQGFPDSYVIPVSDASAYKQFGNSVAVPAIQATGKKILEKLGNLYD

SEQ ID NO: 607 - protein, 521 aa, Arthrobacter luteus
MSKANAKYSFVDLFAGIGGFHAALAATGGVCEYAVEIDREAAAVYERNWNKPALGDITDDANDEGVTLRGYDGPIDVLTGGFPCQPFSKSGAQHGMAETRGTLFWNIARIIEEREPTVLILENVRNLVGPRHRHEWLTIIETLRFFGYEVSGAPAIFSPHLLPAWMGGTPQVRERVFITATLVPERMRDERIPRTETGEIDAEAIGPKPVATMNDRFPIKKGGTELFHPGDRKSGWNLLTSGIIREGDPEPSNVDLRLTETETLWIDAWDDLESTIRRATGRPLEGFPYWADSWTDFRELSRLVVIRGFQAPEREVVGDRKRYVARTDMPEGFVPASVTRPAIDETLPAWKQSHLRRNYDFFERHFAEVVAWAYRWGVYTDLFPASRRKLEWQAQDAPRLWDTVMHFRPSGIRAKRPTYLPALVAITQTSIVGPLERRLSPRETARLQGLPEWFDFGEQRAAATYKQMGNGVNVGVVRHILREHVRRDRALLKLTPAGQRIINAVLADEPDATVGALGAAE

SEQ ID NO: 608 - protein, 330 aa, Haemophilus aegyptius
MNLISLFSGAGGLDLGFQKAGFRIICANEYDKSIWKTYESNHSAKLIKGDISKISSDEFPKCDGIIGGPPCQSWSEGGSLRGIDDPRGKLFYEYIRILKQKKPIFFLAENVKGMMAQRHNKAVQEFIQEFDNAGYDVHIILLNANDYGVAQDRKRVFYIGFRKELNINYLPPIPHLIKPTFKDVIWDLKDNPIPALDKNKTNGNKCIYPNHEYFIGSYSTIFMSRNRVRQWNEPAFTVQASGRQCQLHPQAPVMLKVSKNLNKFVEGKEHLYRRLTVRECARVQGFPDDFIFHYESLNDGYKMIGNAVPVNLAYEIAKTIKSALEICKGN

SEQ ID NO: 609 - protein, 327 aa, Haemophilus haemolyticus
MIEIKDKQLTGLRFIDLFAGLGGFRLALESCGAECVYSNEWDKYAQEVYEMNFGEKPEGDITQVNEKTIPDHDILCAGFPCQAFSISGKQKGFEDSRGTLFFDIARIVREKKPKVVFMENVKNFASHDNGNTLEVVKNTMNELDYSFHAKVLNALDYGIPQKRERIYMICFRNDLNIQNFQFPKPFELNTFVKDLLLPDSEVEHLVIDRKDLVMTNQEIEQTTPKTVRLGIVGKGGQGERIYSTRGIAITLSAYGGGIFAKTGGYLVNGKTRKLHPRECARVMGYPDSYKVHPSTSQAYKQFGNSVVINVLQYIAYNIGSSLNFKPY

SEQ ID NO: 610 - protein, 418 aa, Moraxella sp.
MKPEILKLIRSKLDLTQKQASEIIEVSDKTWQQWESGKTEMHPAYYSFLQEKLKDKINFEELSAQKTLQKKIFDKYNQNQITKNAEELAEITHIEERKDAYSSDFKFIDLFSGIGGIRQSFEVNGGKCVFSSEIDPFAKFTYYTNFGVVPFGDITKVEATTIPQHDILCAGFPCQPFSHIGKREGFEHPTQGTMFHEIVRIIETKKTPVLFLENVPGLINHDDGNTLKVIIETLEDMGYKVHHTVLDASHFGIPQKRKRFYLVAFLNQNIHFEFPKPPMISKDIGEVLESDVTGYSISEHLQKSYLFKKDDGKPSLIDKNTTGAVKTLVSTYHKIQRLTGTFVKDGETGIRLLTTNECKAIMGFPKDFVIPVSRTQMYRQMGNSVVVPVVTKIAEQISLALKTVNQQSPQENFELELV

SEQ ID NO: 611 - protein, 537 aa, Ascobolus sp.
MSERRYEAGMTVALHEGSFLKIQRVYIRQYHADNRREHMLVGPLFRRTKYLKALSKKVNEVAIVHESIHVPVQDVIGVRELIITNRPFPECRKGDEHTGRLVCRWVYNLDERAKGREYKKQRYIRRITEAEADPEYRVEDRVLRRRWFQEGYIGDEISYKEHGNGDIVDIRSESPLQVLDGWGGDLVDLENGEETSIPGPCRSASSYGRLMKPPLAQAADSNTSRKYTFGDTFCGGGGVSLGARQAGLEVKWAFDMNPNAGANYRRNFPNTDFFLAEAEQFIQLSVGISQHVDILHLSPPCQTFSRAHTIAGKNDENNEASFFAVVNLIKAVRPRLFTVEETDGIMDRQSRQFIDTALMGITELGYSFRICVLNAIEYGVCQNRKRLIIIGAAPGEELPPFPLPTHQDFFSKDPRRDLLPAVTLDDALSTITPESTDHHLNHVWQPAEWKTPYDAHRPFKNAIRAGGGEYDIYPDGRRKFTVRELACIQGFPDEYEFVGTLTDKRRIIGNAVPPPLSAAIMSTLRQWMTEKDFERME

SEQ ID NO: 612 - protein, 1534 aa, Arabidopsis sp.
MVENGAKAAKRKKRPLPEIQEVEDVPRTRRPRRAAACTSFKEKSIRVCEKSATIEVKKQQIVEEEFLALRLTALETDVEDRPTRRLNDFVLFDSDGVPQPLEMLEIHDIFVSGAILPSDVCTDKEKEKGVRCTSFGRVEHWSISGYEDGSPVIWISTELADYDCRKPAASYRKVYDYFYEKARASVAVYKKLSKSSGGDPDIGLEELLAAVVRSMSSGSKYFSSGAAIIDFVISQGDFIYNQLAGLDETAKKHESSYVEIPVLVALREKSSKIDKPLQRERNPSNGVRIKEVSQVAESEALTSDQLVDGTDDDRRYAILLQDEENRKSMQQPRKNSSSGSASNMFYIKINEDEIANDYPLPSYYKTSEEETDELILYDASYEVQSEHLPHRMLHNWALYNSDLRFISLELLPMKQCDDIDVNIFGSGVVTDDNGSWISLNDPDSGSQSHDPDGMCIFLSQIKEWMIEFGSDDIISISIRTDVAWYRLGKPSKLYAPWWKPVLKTARVGISILTFLRVESRVARLSFADVTKRLSGLQANDKAYISSDPLAVERYLVVHGQIILQLFAVYPDDNVKRCPFVVGLASKLEDRHHTKWIIKKKKISLKELNLNPRAGMAPVASKRKAMQATTTRLVNRIWGEFYSNYSPEDPLQATAAENGEDEVEEEGGNGEEEVEEEGENGLTEDTVPEPVEVQKPHTPKKIRGSSGKREIKWDGESLGKTSAGEPLYQQALVGGEMVAVGGAVTLEVDDPDEMPAIYFVEYMFESTDHCKMLHGRFLQRGSMTVLGNAANERELFLTNECMTTQLKDIKGVASFEIRSRPWGHQYRKKNITADKLDWARALERKVKDLPTEYYCKSLYSPERGGFFSLPLSDIGRSSGFCTSCKIREDEEKRSTIKLNVSKTGFFINGIEYSVEDFVYVNPDSIGGLKEGSKTSFKSGRNIGLRAYVVCQLLEIVPKESRKADLGSFDVKVRRFYRPEDVSAEKAYASDIQELYFSQDTVVLPPGALEGKCEVRKKSDMPLSREYPISDHIFFCDLFFDTSKGSLKQLPANMKPKFSTIKDDTLLRKKKGKGVESEIESEIVKPVEPPKEIRLATLDIFAGCGGLSHGLKKAGVSDAKWAIEYEEPAGQAFKQNHPESTVFVDNCNVILRAIMEKGGDQDDCVSTTEANELAAKLTEEQKSTLPLPGQVDFINGGPPCQGFSGMNRFNQSSWSKVQCEMILAFLSFADYFRPRYFLLENVRTFVSFNKGQTFQLTLASLLEMGYQVRFGILEAGAYGVSQSRKRAFIWAAAPEEVLPEWPEPMHVFGVPKLKISLSQGLHYAAVRSTALGAPFRPITVRDTIGDLPSVENGDSRTNKEYKEVAVSWFQKEIRGNTIALTDHICKAMNELNLIRCKLIPTRPGADWHDLPKRKVTLSDGRVEEMIPFCLPNTAERHNGWKGLYGRLDWQGNFPTSVTDPQPMGKVGMCFHPEQHRILTVRECARSQGFPDSYEFAGNINHKHRQIGNAVPPPLAFALGRKLKEALHLKKSPQHQP

SEQ ID NO: 613 - protein, 1356 aa, Ascobolus sp.
MELTPELSGVSTDLGGGGSIFAHWRMKEESPAPTEILDDLNVLEWEKTTRDYSKEDLRIADQLFSIEDEHQSLPFETADAEDGTPTEEEEEKELPMRTLDNFVLYDASDLELAALDLIGTELNIHAVGTVGPIYTEGEEDEQEDEDEDVSPPVRTGTQATSASVTQMTVELYIRNIVQYEFCFNDDGTVETWIQTTNAHYKLLQPAKCYTSLYRPVNDCLNVITAIITLAPESTTMSLKDLLKVMDDKAQAVSYEEVERMSEFIVQHLDQWMETAPKKKSKLIEKSKVYIDLNNLAGIDMVSGVRPPPVRRVTGRSSAPKKRIVRNMNDAVLLHQNETTVTNWIHQLSAGMFGRALNVLGAETADVENLTCDPASAKFVVPQRRLHKRLKWETRGHIPVSEEEYKHIYQGKKYAKFFEAVRAVDESKLTIKLGDLVYVLDQDPKVTQTQFATAGREGRKKGAEKEKIQVRFGRVLSIRQPDSNSKDAQNVFIHVQWLVLGCDTILQEMASRRELFLTDSCDTVFADVIYGVAKLTPLGAKDIPTVEFHESMATMMGENEFFVRFKYNYQDGSFTDLKDVDAEQIGTLQPRVNTHRNPGYCSNCRIKYDNERTGDKWIYENDTEGEPRLFRSSKGWCIYAQEFVYLQPVEKQPGTTFRVGYISEINKSSVIVELLARVDDDDKSGHISYSDPRHLYFTGTDIKVTFDKIIRKCFVFHDSGDQKAKAPLMYGTLQRDLYYYRYEKRKGKAELVPVREIRSIHEQTLNDWESRTQIERHGAVSGKKLKGLDIFAGCGGLTLGLDLSGAVDTKWDIEFAPSAANTLALNFPDAQVFNQCANVLLSRAIQSEDEGSLDIEYDLQGRVLPDLPKKGEVDFIYGGPPCQGFSGVNRYKKGNDIKNSLVATFLSYVDHYKPRFVLLENVKGLITTKLGNSKNAEGKWEGGISNGVVKFIYRTLISMNYQCRIGLVQSGEYGVPQSRPRVIFLAARMGERLPDLPEPMHAFEVLDSQYALPHIKRYHTTQNGVAPLPRITIGEAVSDLPKFQYANPGVWPRHDPYSSAKAQPSDKTIEKFSVSKATSFVGYLLQPYHSRPQSEFQRRLRTKLVPSDEPAEKTSLLTTKLVTAHVTRLFNKETTQRIVCVPMWPGADHRSLPKEMRPWCLVDPNSQAEKHRFWPGLFGRLGMEDFFSTALTDVQPCGKQGKVLHPTQRRVYTVRELARAQGFPDWFAFTDGDADSGLGGVKKWHRNIGNAVPVPLGEQIGRCIGYSVWWKDDMIAQLREDGADEDEEMIDGNDQWVEELNTQMAADMPGLPLLVTHLLNLCVYRRLYGPNAKEFLPARVYDKKLEGGRRRLVWAML

SEQ ID NO: 614 - protein, 1454 aa, unidentified
  source 1..1454: note = Neurospora
MDSPDRSHGGMFIDVPAETMGFQEDYLDMFASVLSQGLAKEGDYAHHQPLPAGKEECLEPIAVATTITPSPDDPQLQLQLELEQQFQTESGLNGVDPAPAPESEDEADLPDGFSDESPDDDFVVQRSKHITVDLPVSTLINPRSTFQRIDENDNLVPPPQSTPERVAVEDLLKAAKAAGKNKEDYIEFELHDFNFYVNYAYHPQEMRPIQLVATKVLHDKYYFDGVLKYGNTKHYVTGMQVLELPVGNYGASLHSVKGQIWVRSKHNAKKEIYYLLKKPAFEYQRYYQPFLWIADLGKHVVDYCTRMVERKREVTLGCFKSDFIQWASKAHGKSKAFQNWRAQHPSDDFRTSVAANIGYIWKEINGVAGAKRAAGDQLFRELMIVKPGQYFRQEVPPGPVVTEGDRTVAATIVTPYIKECFGHMILGKVLRLAGEDAEKEKEVKLAKRLKIENKNATKADTKDDMKNDTATESLPTPLRSLPVQVLEATPIESDIVSIVSSDLPPSENNPPPLTNGSVKPKAKANPKPKPSTQPLHAAHVKYLSQELVNKIKVGDVISTPRDDSSNTDTKWKPTDTDDHRWFGLVQRVHTAKTKSSGRGLNSKSFDVIWFYRPEDTPCCAMKYKWRNELFLSNHCTCQEGHHARVKGNEVLAVHPVDWFGTPESNKGEFFVRQLYESEQRRWITLQKDHLTCYHNQPPKPPTAPYKPGDTVLATLSPSDKFSDPYEVVEYFTQGEKETAFVRLRKLLRRRKVDRQDAPANELVYTEDLVDVRAERIVGKCIMRCFRPDERVPSPYDRGGTGNMFFITHRQDHGRCVPLDTLPPTLRQGFNPLGNLGKPKLRGMDLYCGGGNFGRGLEEGGVVEMRWANDIWDKAIHTYMANTPDPNKTNPFLGSVDDLLRLALEGKFSDNVPRPGEVDFIAAGSPCPGFSLLTQDKKVLNQVKNQSLVASFASFVDFYRPKYGVLENVSGIVQTFVNRKQDVLSQLFCALVGMGYQAQLILGDAWAHGAPQSRERVFLYFAAPGLPLPDPPLPSHSHYRVKNRNIGFLCNGESYVQRSFIPTAFKFVSAGEGTADLPKIGDGKPDACVRFPDHRLASGITPYIRAQYACIPTHPYGMNFIKAWNNGNGVMSKSDRDLFPSEGKTRTSDASVGWKRLNPKTLFPTVTTTSNPSDARMGPGLHWDEDRPYTVQEMRRAQGYLDEEVLVGRTTDQWKLVGNSVSRHMALAIGLKFREAWLGTLYDESAVVATATATATTAAAVGVTVPVMEEPGIGTTESSRPSRSPVHTAVDLDDSKSERSRSTTPATVLSTSSAAGDGSANAAGLEDDDNDDMEMMEVTRKRSSPAVDEEGMRPSKVQKVEVTVASPASRRSSRQASRNPTASPSSKASKATTHEAPAPEELESDAESYSETYDKEGFDGDYHSGHEDQYSEEDEEEEYAEPETMTVNGMTIVKL

SEQ ID NO: 615 - protein, 345 aa, Drosophila sp.
MVFRVLELFSGIGGMHYAFNYAQLDGQIVAALDVNTVANAVYAHNYGSNLVKTRNIQSLSVKEVTKLQANMLLMSPPCQPHTRQGLQRDTEDKRSDALTHLCGLIPECQELEYILMENVKGFESSQARNQFIESLERSGFHWREFILTPTQFNVPNTRYRYYCIARKGADFPFAGGKIWEEMPGAIAQNQGLSQIAEIVEENVSPDFLVPDDVLTKRVLVMDIIHPAQSRSMCFTKGYTHYTEGTGSAYTPLSEDESHRIFELVKEIDTSNQDASKSEKILQQRLDLLHQVRLRYFTPREVARLMSFPENFEFPPETTNRQKYRLLGNSINVKVVGELIKLLTIK

SEQ ID NO: 616 - protein, 330 aa, Schizosaccharomyces pombe
MLSTKRLRVLELYSGIGGMHYALNLANIPADIVCAIDINPQANEIYNLNHGKLAKHMDISTLTAKDFDAFDCKLWTMSPSCQPFTRIGNRKDILDPRSQAFLNILNVLPHVNNLPEYILIENVQGFEESKAAEECRKVLRNCGYNLIEGILSPNQFNIPNSRSRWYGLARLNFKGEWSIDDVFQFSEVAQKEGEVKRIRDYLEIERDWSSYMVLESVLNKWGHQFDIVKPDSSSCCCFTRGYTHLVQGAGSILQMSDHENTHEQFERNRMALQLRYFTAREVARLMGFPESLEWSKSNVTEKCMYRLLGNSINVKVVSYLISLLLEPLNF

SEQ ID NO: 617 - protein, 624 aa, Arabidopsis sp.
MVMSHIFLISQIQEVEHGDSDDVNWNTDDDELAIDNFQFSPSPVHISATSPNSIQNRISDETVASFVEMGFSTQMIARAIEETAGANMEPMMILETLFNYSASTEASSSKSKVINHFIAMGFPEEHVIKAMQEHGDEDVGEITNALLTYAEVDKLRESEDMNININDDDDDNLYSLSSDDEEDELNNSSNEDRILQALIKMGYLREDAAIAIERCGEDASMEEVVDFICAAQMARQFDEIYAEPDKKELMNNNKKRRTYTETPRKPNTDQLISLPKEMIGFGVPNHPGLMMHRPVPIPDIARGPPFFYYENVAMTPKGVWAKISSHLYDIVPEFVDSKHFCAAARKRGYIHNLPIQNRFQIQPPQHNTIQEAFPLTKRWWPSWDGRTKLNCLLTCIASSRLTEKIREALERYDGETPLDVQKWVMYECKKWNLVWVGKNKLAPLDADEMEKLLGFPRDHTRGGGISTTDRYKSLGNSFQVDTVAYHLSVLKPLFPNGINVLSLFTGIGGGEVALHRLQIKMNVVVSVEISDANRNILRSFWEQTNQKGILREFKDVQKLDDNTIERLMDEYGGFDLVIGGSPCNNLAGGNRHHRVGLGGEHSSLFFDYCRILEAVRRKARHMRR

SEQ ID NO: 618 - protein, 626 aa, Arabidopsis sp.
MVIWNNDDDDFLEIDNFQSSPRSSPIHAMQCRVENLAGVAVTTSSLSSPTETTDLVQMGFSDEVFATLFDMGFPVEMISRAIKETGPNVETSVIIDTISKYSSDCEAGSSKSKAIDHFLAMGFDEEKVVKAIQEHGEDNMEAIANALLSCPEAKKLPAAVEEEDGIDWSSSDDDTNYTDMLNSDDEKDPNSNENGSKIRSLVKMGFSELEASLAVERCGENVDIAELTDFLCAAQMAREFSEFYTEHEEQKPRHNIKKRRFESKGEPRSSVDDEPIRLPNPMIGFGVPNEPGLITHRSLPELARGPPFFYYENVALTPKGVWETISRHLFEIPPEFVDSKYFCVAARKRGYIHNLPINNRFQIQPPPKYTIHDAFPLSKRWWPEWDKRTKLNCILTCTGSAQLTNRIRVALEPYNEEPEPPKHVQRYVIDQCKKWNLVWVGKNKAAPLEPDEMESILGFPKNHTRGGGMSRTERFKSLGNSFQVDTVAYHLSVLKPIFPHGINVLSLFTGIGGGEVALHRLQIKMKLVVSVEISKVNRNILKDFWEQTNQTGELIEFSDIQHLTNDTIEGLMEKYGGFDLVIGGSPCNNLAGGNRVSRVGLEGDQSSLFFEYCRILEVVRARMRGS

SEQ ID NO: 619 - protein, 791 aa, Arabidopsis sp.
MAARNKQKKRAEPESDLCFAGKPMSVVESTIRWPHRYQSKKTKLQAPTKKPANKGGKKEDEEIIKQAKCHFDKALVDGVLINLNDDVYVTGLPGKLKFIAKVIELFEADDGVPYCRFRWYYRPEDTLIERFSHLVQPKRVFLSNDENDNPLTCIWSKVNIAKVPLPKITSRIEQRVIPPCDYYYDMKYEVPYLNFTSADDGSDASSSLSSDSALNCFENLHKDEKFLLDLYSGCGAMSTGFCMGASISGVKLITKWSVDINKFACDSLKLNHPETEVRNEAAEDFLALLKEWKRLCEKFSLVSSTEPVESISELEDEEVEENDDIDEASTGAELEPGEFEVEKFLGIMFGDPQGTGEKTLQLMVRWKGYNSSYDTWEPYSGLGNCKEKLKEYVIDGFKSHLLPLPGTVYTVCGGPPCQGISGYNRYRNNEAPLEDQKNQQLLVFLDIIDFLKPNYVLMENVVDLLRFSKGFLARHAVASFVAMNYQTRLGMMAAGSYGLPQLRNRVFLWAAQPSEKLPPYPLPTHEVAKKFNTPKEFKDLQVGRIQMEFLKLDNALTLADAISDLPPVTNYVANDVMDYNDAAPKTEFENFISLKRSETLLPAFGGDPTRRLFDHQPLVLGDDDLERVSYIPKQKGANYRDMPGVLVHNNKAEINPRFRAKLKSGKNVVPAYAISFIKGKSKKPFGRLWGDEIVNTVVTRAEPHNQCVIHPMQNRVLSVRENARLQGFPDCYKLCGTIKEKYIQVGNAVAVPVGVALGYAFGMASQGLTDDEPVIKLPFKYPECMQAKDQI

SEQ ID NO: 620 - protein, 1295 aa, Arabidopsis sp.
MLSPAKCESEEAQAPLDLHSSSRSEPECLSLVLWCPNPEEAAPSSTRELIKLPDNGEMSLRRSTTLNCNSPEENGGEGRVSQRKSSRGKSQPLLMLTNGCQLRRSPRFRALHANFDNVCSVPVTKGGVSQRKFSRGKSQPLLTLTNGCQLRRSPRFRAVDGNFDSVCSVPVTGKFGSRKRKSNSALDKKESSDSEGLTFKDIAVIAKSLEMEIISECQYKNNVAEGRSRLQDPAKRKVDSDTLLYSSINSSKQSLGSNKRMRRSQRFMKGTENEGEENLGKSKGKGMSLASCSFRRSTRLSGTVETGNTETLNRRKDCGPALCGAEQVRGTERLVQISKKDHCCEAMKKCEGDGLVSSKQELLVFPSGCIKKTVNGCRDRTLGKPRSSGLNTDDIHTSSLKISKNDTSNGLTMTTALVEQDAMESLLQGKTSACGAADKGKTREMHVNSTVIYLSDSDEPSSIEYLNGDNLTQVESGSALSSGGNEGIVSLDLNNPTKSTKRKGKRVTRTAVQEQNKRSICFFIGEPLSCEEAQERWRWRYELKERKSKSRGQQSEDDEDKIVANVECHYSQAKVDGHTFSLGDFAYIKGEEEETHVGQIVEFFKTTDGESYFRVQWFYRATDTIMERQATNHDKRRLFYSTVMNDNPVDCLISKVTVLQVSPRVGLKPNSIKSDYYFDMEYCVEYSTFQTLRNPKTSENKLECCADVVPTESTESILKKKSFSGELPVLDLYSGCGGMSTGLSLGAKISGVDVVTKWAVDQNTAACKSLKLNHPNTQVRNDAAGDFLQLLKEWDKLCKRYVFNNDQRTDTLRSVNSTKETSGSSSSSDDDSDSEEYEVEKLVDICFGDHDKTGKNGLKFKVHWKGYRSDEDTWELAEELSNCQDAIREFVTSGFKSKILPLPGRVGVICGGPPCQGISGYNRHRNVDSPLNDERNQQIIVFMDIVEYLKPSYVLMENVVDILRMDKGSLGRYALSRLVNMRYQARLGIMTAGCYGLSQFRSRVFMWGAVPNKNLPPFPLPTHDVIVRYGLPLEFERNVVAYAEGQPRKLEKALVLKDAISDLPHVSNDEDREKLPYESLPKTDFQRYIRSTKRDLTGSAIDNCNKRTMLLHDHRPFHINEDDYARVCQIPKRKGANFRDLPGLIVRNNTVCRDPSMEPVILPSGKPLVPGYVFTFQQGKSKRPFARLWWDETVPTVLTVPTCHSQALLHPEQDRVLTIRESARLQGFPDYFQFCGTIKERYCQIGNAVAVSVSRALGYSLGMAFRGLARDEHLIKLPQNFSHSTYPQLQETIPH

SEQ ID NO: 621 - protein, 839 aa, Arabidopsis sp.
MAPKRKRPATKDDTTKSIPKPKKRAPKRAKTVKEEPVTVVEEGEKHVARFLDEPIPESEAKSTWPDRYKPIEVQPPKASSRKKTKDDEKVEIIRARCHYRRAIVDERQIYELNDDAYVQSGEGKDPFICKIIEMFEGANGKLYFTARWFYRPSDTVMKEFEILIKKKRVFFSEIQDTNELGLLEKKLNILMIPLNENTKETIPATENCDFFCDMNYFLPYDTFEAIQQETMMAISESSTISSDTDIREGAAAISEIGECSQETEGHKKATLLDLYSGCGAMSTGLCMGAQLSGLNLVTKWAVDMNAHACKSLQHNHPETNVRNMTAEDFLFLLKEWEKLCIHFSLRNSPNSEEYANLHGLNNVEDNEDVSEESENEDDGEVFTVDKIVGISFGVPKKLLKRGLYLKVRWLNYDDSHDTWEPIEGLSNCRGKIEEFVKLGYKSGILPLPGGVDVVCGGPPCQGISGHNRFRNLLDPLEDQKNKQLLVYMNIVEYLKPKFVLMENVVDMLKMAKGYLARFAVGRLLQMNYQVRNGMMAAGAYGLAQFRLRFFLWGALPSEIIPQFPLPTHDLVHRGNIVKEFQGNIVAYDEGHTVKLADKLLLKDVISDLPAVANSEKRDEITYDKDPTTPFQKFIRLRKDEASGSQSKSKSKKHVLYDHHPLNLNINDYERVCQVPKRKGANFRDFPGVIVGPGNVVKLEEGKERVKLESGKTLVPDYALTYVDGKSCKPFGRLWWDEIVPTVVTRAEPHNQVIIHPEQNRVLSIRENARLQGFPDDYKLFGPPKQKYIQVGNAVAVPVAKALGYALGTAFQGLAVGKDPLLTLPEGFAFMKPTLPSELA

SEQ ID NO: 622 - protein, 142 aa, unidentified
  source 1..142: note = Neurospora
MAEQNPFVIDDEDDVIQIHDEEEVEEEVAEVIDITEDDIEPSELDRAFGSRPKEETLPSLLLRDQGFIVRPGMTVELKAPIGRFAISFVRVNSIVKVRQAHVNNVTIRGHGFTRAKEMNGMLPKQLNECCLVASIDTRDPRP

SEQ ID NO: 623 - protein, 529 aa, Escherichia coli
MNNNDLVAKLWKLCDNLRDGGVSYQNYVNELASLLFLKMCKETGQEAEYLPEGYRWDDLKSRIGQEQLQFYRKMLVHLGEDDKKLVQAVFHNVSTTITEPKQITALVSNMDSLDWYNGAHGKSRDDFGDMYEGLLQKNANETKSGAGQYFTPRPLIKTIIHLLKPQPREVVQDPAAGTAGFLIEADRYVKSQTNDLDDLDGDTQDFQIHRAFIGLELVPGTRRLALMNCLLHDIEGNLDHGGAIRLGNTLGSDGENLPKAHIVATNPPFGSAAGTNITRTFVHPTSNKQLCFMQHIIETLHPGGRAAVVVPDNVLFEGGKGTDIRRDLMDKCHLHTILRLPTGIFYAQGVKTNVLFFTKGTVANPNQDKNCTDDVWVYDLRTNMPSFGKRTPFTDEHLQPFERVYGEDPHGLSPRTEGEWSFNAEETEVADSEENKNTDQHLATSRWRKFSREWIRTAKSDSLDISWLKDKDSIDADSLPEPDVLAAEAMGELVQALSELDALMRELGASDEADLQRQLLEEAFGGVKE

SEQ ID NO: 624 - protein, 464 aa, Escherichia coli
MSAGKLPEGWVIAPVSTVTTLIRGVTYKKEQAINYLKDDYLPLIRANNIQNGKFDTTDLVFVPKNLVKESQKISPEDIVIAMSSGSKSVVGKSAHQHLPFECSFGAFCGVLRPEKLIFSGFIAHFTKSSLYRNKISSLSAGANINNIKPASFDLINIPIPPLAEQKIIAEKLDTLLAQVDSTKARFEQIPQILKRFRQAVLGGAVNGKLTEKWRNFEPQHSVFKKLNFESILTELRNGLSSKPNESGVGHPILRISSVRAGHVDQNDIRFLECSESELNRHKLQDGDLLFTRYNGSLEFVGVCGLLKKLQHQNLLYPDKLIRARLTKDALPEYIEIFFSSPSARNAMMNCVKTTSGQKGISGKDIKSQVVLLPPVKEQAEIVRRVEQLFAYADTIEKQVNNALARVNNLTQSILAKAFRGELTAQWRAENPDLISGENSAAALLEKIKAERAASGGKKASRKKS

SEQ ID NO: 625 - protein, 421 aa, Thermus aquaticus
MGLPPLLSLPSNSAPRSLGRVETPPEVVDFMVSLAEAPRGGRVLEPACAHGPFLRAFREAHGTAYRFVGVEIDPKALDLPPWAEGILADFLLWEPGEAFDLILGNPPYGIVGEASKYPIHVFKAVKDLYKKAFSTWKGKYNLYGAFLEKAVRLLKPGGVLVFVVPATWLVLEDFALLREFLAREGKTSVYYLGEVFPQKKVSAVVIRFQKSGKGLSLWDTQESESGFTPILWAEYPHWEGEIIRFETEETRKLEISGMPLGDLFHIRFAARSPEFKKHPAVRKEPGPGLVPVLTGRNLKPGWVDYEKNHSGLWMPKERAKELRDFYATPHLVVAHTKGTRVVAAWDERAYPWREEFHLLPKEGVRLDPSSLVQWLNSEAMQKHVRTLYRDFVPHLTLRMLERLPVRREYGFHTSPESARNF

SEQ ID NO: 626 - protein, 278 aa, Escherichia coli
MKKNRAFLKWAGGKYPLLDDIKRHLPKGECLVEPFVGAGSVFLNTDFSRYILADINSDLISLYNIVKMRTDEYVQAARELFVPETNCAEVYYQFREEFNKSQDPFRRAVLFLYLNRYGYNGLCRYNLRGEFNVPFGRYKKPYFPEAELYHFAEKAQNAFFYCESYADSMARADDASVVYCDPPYAPLSATANFTAYHTNSFTLEQQAHLAEIAEGLVERHIPVLISNHDTMLTREWYQRAKLHVVKVRRSISSNGGTRKKVDELLALYKPGVVSPAKK

SEQ ID NO: 627 - protein, 358 aa, Caulobacter crescentus
MKFGPETIIHGDCIEQMNALPEKSVDLIFADPPYNLQLGGDLLRPDNSKVDAVDDHWDQFESFAAYDKFTREWLKAARRVLKDDGAIWVIGSYHNIFRVGVAVQDLGFWILNDIVWRKSNPMPNFKGTRFANAHETLIWASKSQNAKRYTFNYDALKMANDEVQMRSDWTIPLCTGEERIKGADGQKAHPTQKPEALLYRVILSTTKPGDVILDPFFGVGTTGAAAKRLGRKFIGIEREAEYLEHAKARIAKVVPIAPEDLDVMGSKRAEPRVPFGTIVEAGLLSPGDTLYCSKGTHVAKVRPDGSITVGDLSGSIHKIGALVQSAPACNGWTYWHFKTDAGLAPIDVLRAQVRAGMN

SEQ ID NO: 628 - protein, 577 aa, Clostridioides difficile
MDDISQDNFLLSKEYENSLDVDTKKASGIYYTPKIIVDYIVKKTLKNHDIIKNPYPRILDISCGCGNFLLEVYDILYDLFEENIYELKKKYDENYWTVDNIHRHILNYCIYGADIDEKAISILKDSLTNKKVVNDLDESDIKINLFCCDSLKKKWRYKFDYIVGNPPYIGHKKLEKKYKKFLLEKYSEVYKDKADLYFCFYKKIIDILKQGGIGSVITPRYFLESLSGKDLREYIKSNVNVQEIVDFLGANIFKNIGVSSCILTFDKKKTKETYIDVFKIKNEDICINKFETLEELLKSSKFEHFNINQRLLSDEWILVNKDDETFYNKIQEKCKYSLEDIAISFQGIITGCDKAFILSKDDVKLNLVDDKFLKCWIKSKNINKYIVDKSEYRLIYSNDIDNENTNKRILDEIIGLYKTKLENRRECKSGIRKWYELQWGREKLFFERKKIMYPYKSNENRFAIDYDNNFSSADVYSFFIKEEYLDKFSYEYLVGILNSSVYDKYFKITAKKMSKNIYDYYPNKVMKIRIFRDNNYEEIENLSKQIISILLNKSIDKGKVEKLQIKMDNLIMDSLGI

SEQ ID NO: 629 - protein, 835 aa, Homo sapiens
MAASAAAASAAAASAASGSPGPGEGSAGGEKRSTAPSAAASASASAAASSPAGGGAEALELLEHCGVCRERLRPEREPRLLPCLHSACSACLGPAAPAAANSSGDGGAAGDGTVVDCPVCKQQCFSKDIVENYFMRDSGSKAATDAQDANQCCTSCEDNAPATSYCVECSEPLCETCVEAHQRVKYTKDHTVRSTGPAKSRDGERTVYCNVHKHEPLVLFCESCDTLTCRDCQLNAHKDHQYQFLEDAVRNQRKLLASLVKRLGDKHATLQKSTKEVRSSIRQVSDVQKRVQVDVKMAILQIMKELNKRGRVLVNDAQKVTEGQQERLERQHWTMTKIQKHQEHILRFASWALESDNNTALLLSKKLIYFQLHRALKMIVDPVEPHGEMKFQWDLNAWTKSAEAFGKIVAERPGTNSTGPAPMAPPRAPGPLSKQGSGSSQPMEVQEGYGFGSGDDPYSSAEPHVSGVKRSRSGEGEVSGLMRKVPRVSLERLDLDLTADSQPPVFKVFPGSTTEDYNLIVIERGAAAAATGQPGTAPAGTPGAPPLAGMAIVKEEETEAAIGAPPTATEGPETKPVLMALAEGPGAEGPRLASPSGSTSSGLEVVAPEGTSAPGGGPGTLDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNEAFGDTKFSAVLVEPPPMSLPGAGLSSQELSGGPGDGP

SEQ ID NO: 630 - protein, 486 aa, Homo sapiens
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 631 - protein, 4 aa, synthetic construct
SGGS

SEQ ID NO: 632 - protein, 24 aa, synthetic construct
SGGSSGSETPGTSESATPESSGGS

SEQ ID NO: 633 - protein, 32 aa, synthetic construct
SGGSSGGSSGSETPGTSESATPESSGGSSGGS

SEQ ID NO: 634 - protein, 104 aa, synthetic construct
GGSGGSPGSPAGSPTSTEEGTSESATPESGPGTSTEPSEGSAPGSPAGSPTSTEEGTSTEPSEGSAPGTSTEPSEGSAPGTSESATPESGPGSEPATSGGSGGS

SEQ ID NO: 635 - protein, 5 aa, synthetic construct
GSGGG

SEQ ID NO: 636 - protein, 20 aa, synthetic construct
GGGGSGGGGSGGGGSGGGGS

SEQ ID NO: 637 - protein, 27 aa, synthetic construct
SSGNSNANSRGPSFSSGLVPLSLRGSH

SEQ ID NO: 638 - protein, 16 aa, synthetic construct
SGSETPGTSESATPES

SEQ ID NO: 639 - protein, 24 aa, synthetic construct
SGGSSGGSSGSETPGTSESATPES

SEQ ID NO: 640 - protein, 40 aa, synthetic construct
SGGSSGGSSGSETPGTSESATPESSGGSSGGSSGGSSGGS

SEQ ID NO: 641 - protein, 64 aa, synthetic construct
SGGSSGGSSGSETPGTSESATPESSGGSSGGSSGGSSGGSSGSETPGTSESATPESSGGSSGGS

SEQ ID NO: 642 - protein, 92 aa, synthetic construct
PGSPAGSPTSTEEGTSESATPESGPGTSTEPSEGSAPGSPAGSPTSTEEGTSTEPSEGSAPGTSTEPSEGSAPGTSESATPESGPGSEPATS

SEQ ID NO: 643 - protein, 80 aa, synthetic construct
GGPSSGAPPPSGGSPAGSPTSTEEGTSESATPESGPGTSTEPSEGSAPGSPAGSPTSTEEGTSTEPSEGSAPGTSTEPSE

SEQ ID NO: 644 - protein, 7 aa, synthetic construct
PKKKRKV

SEQ ID NO: 645 - protein, 20 aa, synthetic construct
AVKRPAATKKAGQAKKKKLD

SEQ ID NO: 646 - protein, 25 aa, synthetic construct
MSRRRKANPTKLSENAKKLAKEVEN

SEQ ID NO: 647 - protein, 9 aa, synthetic construct
PAAKRVKLD

SEQ ID NO: 648 - protein, 9 aa, synthetic construct
KLKIKRPVK

SEQ ID NO: 649 - protein, 30 aa, synthetic construct
MDSLLMNRRKFLYQFKNVRWAKGRRETYLC

SEQ ID NO: 650 - protein, 665 aa, synthetic construct
  VARIANT 27..28: note = May be either TR, LR or LK
  VARIANT 55..56: note = May be either TR, LR or LK
  VARIANT 328..329: note = May be either TR, LR or LK
  VARIANT 356..357: note = May be either TR, LR or LK
  VARIANT 630..631: note = May be either TR, LR or LK
  VARIANT 658..659: note = May be either TR, LR or LK
  VARIANT 64..308: note = May be absent
  VARIANT 365..609: note = May be absent
SRPGERPFQCRICMRNFSXXXXXXXHXXTHTGEKPFQCRICMRNFSXXXXXXXHXXTHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXFQCRICMRNFSXXXXXXXHXXTHTGEKPFQCRICMRNFSXXXXXXXHXXTHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFQCRICMRNFSXXXXXXXHXXTHLRGS

SEQ ID NO: 651 - protein, 6 aa, synthetic construct
TGSQKP

SEQ ID NO: 652 - protein, 9 aa, synthetic construct
TGGGGSQKP

SEQ ID NO: 653 - DNA, 906 bp, synthetic construct
  source 1..906: mol_type = other DNA
tacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtg

SEQ ID NO: 654 - DNA, 81 bp, synthetic construct
  source 1..81: mol_type = other DNA
agcagcggcaactccaatgccaacagccggggcccctctttcagctccggattggtgcctctgagcctgaggggctcccac

SEQ ID NO: 655 - DNA, 1158 bp, synthetic construct
  source 1..1158: mol_type = other DNA
atggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctg

SEQ ID NO: 656 - DNA, 63 bp, synthetic construct
  source 1..63: mol_type = other DNA
ggtgttgatggctcctctggttctgaaactcctggtacttctgagtctgctactcccgaatct

SEQ ID NO: 657 - protein, 25 aa, synthetic construct
CXXXXCXXXXXXXXXXXXHXXXXXH

SEQ ID NO: 658 - protein, 2272 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSXXXXXXXHXXTHTGEKPFQCRICMRNFSXXXXXXXHXXTHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFQCRICMRNFSXXXXXXXHXXTHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFQCRICMRNFSXXXXXXXHXXTHLRGSGVDGSSGSETPGTSESATPESERPFQCRICMRNFSXXXXXXXHXXTHTGEKPFACDICGRKFAXXXXXXXHXXIHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFACDICGRKFAXXXXXXXHXXIHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFACDICGRKFAXXXXXXXHXXIHLRQKDAARGSSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESAPKKKRKVPKKKRKV

SEQ ID NO: 659 - protein, 2434 aa, synthetic construct
MPKKKRKVPKKKRKVDSVAFEDVAVNFTLEEWALLDPSQKNLYRDVMRETFRNLASVGKQWEDQNIEDPFKIPRRNISHIPERLCESKEGGQGEESASGSETPGTSESATPESTGERPFQCRICMRNFSXXXXXXXHXXTHTGEKPFACDICGRKFAXXXXXXXHXXIHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFACDICGRKFAXXXXXXXHXXIHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFACDICGRKFAXXXXXXXHXXIHLRQKDAARGSSPKKKRKVGVDGSGGPSSGAPPPSGGSPAGSPTSTEEGTSESATPESGPGTSTEPSEGSAPGSPAGSPTSTEEGTSTEPSEGSAPGTSTEPSEYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSXXXXXXXHXXTHTGEKPFQCRICMRNFSXXXXXXXHXXTHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFQCRICMRNFSXXXXXXXHXXTHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPFQCRICMRNFSXXXXXXXHXXTHTGEKPFQCRICMRNFSXXXXXXXHXXTHLRGSGVDGSSGSETPGTSESATPESDSVAFEDVAVNFTLEEWALLDPSQKNLYRDVMRETFRNLASVGKQWEDQNIEDPFKIPRRNISHIPERLCESKEGGQGEESAPKKKRKVPKKKRKV

SEQ ID NO: 660 - DNA, 63 bp, synthetic construct; identical to SEQ ID NO: 656
  source 1..63: mol_type = other DNA

SEQ ID NO: 661 - DNA, 63 bp, synthetic construct
  source 1..63: mol_type = other DNA
ggagtcgacggatccagcggctccgagaccccaggcacatctgagagcgccacccctgagtcc

SEQ ID NO: 662 - DNA, 240 bp, synthetic construct
  source 1..240: mol_type = other DNA
gactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaa

SEQ ID NO: 663 - DNA, 24 bp, synthetic construct
  source 1..24: mol_type = other DNA
gattacaaagatgatgacgataaa

SEQ ID NO: 664 - protein, 150 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 665 - protein, 150 aa, synthetic construct
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 666 - DNA, 599 bp, synthetic construct
  source 1..599: mol_type = other DNA
aaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaatt

SEQ ID NO: 667 - protein, 8 aa, synthetic construct
MPKKKRKV

SEQ ID NO: 668 - DNA, 5770 bp, synthetic construct
  source 1..5770: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataagggggcgctcgagcaggttcagaaggagatcaaaaacccccaaggatcaaacatgccaaaaaagaagagaaaggtaccgaagaaaaaaagaaaggtatacgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcggagtcgacggatccagcggctccgagaccccaggcacatctgagagcgccacccctgagtccgagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagcccaagaagaagaggaaggtgtcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagcggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtgaggatcctgagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaatt

SEQ ID NO: 669 - protein, 7 aa, synthetic construct
TPSKLDR

SEQ ID NO: 670 - protein, 7 aa, synthetic construct
HKQHRDA

SEQ ID NO: 671 - protein, 7 aa, synthetic construct
LAENLRR

SEQ ID NO: 672 - protein, 7 aa, synthetic construct
RSANLTR

SEQ ID NO: 673 - protein, 7 aa, synthetic construct
RQDNLGR

SEQ ID NO: 674 - protein, 7 aa, synthetic construct
RSDNLSE

SEQ ID NO: 675 - protein, 7 aa, synthetic construct
DGGNLGR

SEQ ID NO: 676 - protein, 7 aa, synthetic construct
TSANLSR

SEQ ID NO: 677 - protein, 7 aa, synthetic construct
QSPHLKR

SEQ ID NO: 678 - protein, 7 aa, synthetic construct
IRSTLRD

SEQ ID NO: 679 - protein, 7 aa, synthetic construct
QSTSLQR

SEQ ID NO: 680 - protein, 7 aa, synthetic construct
RTPVRMG

SEQ ID NO: 681 - DNA, 3354 bp, synthetic construct
  source 1..3354: mol_type = other DNA
atgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgaggagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctagcggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtga

SEQ ID NO: 682 - DNA, 4021 bp, synthetic construct
  source 1..4021: mol_type = other DNA
gggcgctcgagcaggttcagaaggagatcaaaaacccccaaggatcaaacatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagcggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtgaggatcctgagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaatt

SEQ ID NO: 683 - DNA, 4018 bp, synthetic construct
  source 1..4018: mol_type = other DNA
gggcgctcgagcaggttcagaaggagatcaaaaacccccaaggatcaaacatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgaggagagaccttttcagtgccggatttgtatgcgcaatttctcccacaagcagcaccgcgacgcccatattagaacacatactggagaaaagcccttcgcttgcgacatctgtggacggaagttcgcccgctccgccaacctgacccgccacactaaaatccataccggcagccaaaagccattccaatgtcgcatctgtatgcggaactttagccgctccgacaacctgtccgagcatattcggactcacacaggggagaaaccatttgcatgtgatatctgcggcagaaaattcgctacctccgccaacctgtcccgccataccaagatacacacgggatctcagaagcccttccagtgtcgaatctgcatgcgtaacttcagtatccgctccaccctgcgcgaccacatccgcacccacaccggcgagaagccttttgcctgtgacatttgtgggaggaaatttgcccgcaccccggtgcgcatgggccataccaagatacacctgcgccaaaaagatgcggcccggggatctagcggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtgaggatcctgagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaatt

SEQ ID NO: 684 - protein, 1369 aa, synthetic construct
LEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 685 - protein, 65 aa, synthetic construct
RTLVTFKDVFVDFTREEWKLLDTAQQIVYRNVMLENYKNLVSLGYQLTKPDVILRLEKGEEPWLV

SEQ ID NO: 686 - DNA, 21 bp, synthetic construct
  source 1..21: mol_type = other DNA
ccgaagaaaaagcgcaaggtc

SEQ ID NO: 687 - DNA, 903 bp, synthetic construct
  source 1..903: mol_type = other DNA
aatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtg

SEQ ID NO: 688 - DNA, 4107 bp, synthetic construct
  source 1..4107: mol_type = other DNA
ctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 689 - DNA, 195 bp, synthetic construct
  source 1..195: mol_type = other DNA
cggaccctggtgacattcaaggacgtgttcgtggacttcacccgggaggagtggaagctgctggacacagcccagcagatcgtgtacaggaacgtgatgctggagaactataagaatctggtgtctctgggctaccagctgacaaagccagatgtgatcctgcggctggagaagggagaggagccctggctggtg

SEQ ID NO: 690 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 691 - DNA, 4101 bp, synthetic construct
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 692 - DNA, 48 bp, synthetic construct
  source 1..48: mol_type = other DNA
agcggaagtgagaccccaggtacatccgaatcagcaacgcctgaaagc

SEQ ID NO: 693 - DNA, 186 bp, synthetic construct
  source 1..186: mol_type = other DNA
cggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 694 - DNA, 8103 bp, synthetic construct
  source 1..8103: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataaggagacccaagctaccggtgccaccatgtacccatacgatgttccagattacgcttcgccgaagaaaaagcgcaaggtcaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtcccggaccctggtgacattcaaggacgtgttcgtggacttcacccgggaggagtggaagctgctggacacagcccagcagatcgtgtacaggaacgtgatgctggagaactataagaatctggtgtctctgggctaccagctgacaaagccagatgtgatcctgcggctggagaagggagaggagccctggctggtgtagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaatt

SEQ ID NO: 695 - DNA, 26 bp, synthetic construct
  source 1..26: mol_type = other DNA
agacccaagctaccggtgccaccatg

SEQ ID NO: 696 - DNA, 5763 bp, synthetic construct
  source 1..5763: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataaggagacccaagctggctagccgccaccatgggtaccatgccaaaaaagaagagaaaggtaccgaagaaaaaaagaaaggtatacgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcggaagtgagaccccaggtacatccgaatcagcaacgcctgaaagcaccggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcgctgattacaaagatgatgacgataaagccccaaaaaagaagagaaaggtaccgaagaaaaaaagaaaggtctgaggatcctgagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaatt

SEQ ID NO: 697 - DNA, 34 bp, synthetic construct
  source 1..34: mol_type = other DNA
agacccaagctggctagccgccaccatgggtacc

SEQ ID NO: 698 - RNA, 20 nt, synthetic construct
  source 1..20: mol_type = other RNA
gcaacaccgcctagaccgac

SEQ ID NO: 699 - RNA, 92 nt, synthetic construct
  source 1..92: mol_type = other RNA
gtttaagagctaagctggaaacagcatagcaagtttaaataaggctagtccgttatcaacttgaaaaagtggcaccgagtcggtgctttttt

SEQ ID NO: 700 - DNA, 20 bp, synthetic construct
  source 1..20: mol_type = other DNA
gcaacaccgcctagaccgac

SEQ ID NO: 701 - RNA, 20 nt, synthetic construct
  source 1..20: mol_type = other RNA
actgcctggctcactcctcc

SEQ ID NO: 702 - DNA, 20 bp, synthetic construct
  source 1..20: mol_type = other DNA
actgcctggctcactcctcc

SEQ ID NO: 703 - protein, 30 aa, synthetic construct
GGGGGGGGGGGGGGGGGGGGGGGGGGGGGG

SEQ ID NO: 704 - protein, 120 aa, synthetic construct
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 705 - protein, 60 aa, synthetic construct
XPXPXPXPXPXPXPXPXPXPXPXPXPXPXPXPXPXPXPXPXPXPXPXPXPXPXPXPXPXP

SEQ ID NO: 706 - protein, 21 aa, synthetic construct
GGSGGSGGSGGSGGSGGSGGS

SEQ ID NO: 707 - protein, 176 aa, synthetic construct
SRPGERPFQCRICMRNFSRTDTLARHLRTHTGEKPFQCRICMRNFSRKTALNRHLKTHTGSQKPFQCRICMRNFSRNESLKVHLRTHTGEKPFQCRICMRNFSVKNTLTRHLKTHTGSQKPFQCRICMRNFSRREHLVRHLRTHTGEKPFQCRICMRNFSISHNLARHLKTHLRGS

SEQ ID NO: 708 - DNA, 528 bp, synthetic construct
  source 1..528: mol_type = other DNA
tcccggccaggggaacggcccttccagtgtcggatctgcatgagaaacttttcaagaacagacactctggcccgacacctgaggacccacactggagagaaaccctttcagtgcaggatatgtatgcggaatttttccagaaagaccgcacttaatcgacatctcaagacacataccgggagtcagaagcctttccaatgccggatttgcatgaggaacttctcccggaacgaatctctgaaggtgcacctgcgcacacatacaggcgagaagccattccagtgtaggatctgcatgcgcaattttagcgtgaagaatacactgacgagacatctgaagacgcatacaggttcccagaagccctttcagtgcaggatctgcatgaggaattttagtagaagggagcatctcgttagacacctccggacgcatactggagagaagccctttcagtgtaggatttgtatgcggaacttcagcataagccacaatctggcaaggcaccttaaaactcatttgcgcgggtct

SEQ ID NO: 709 - DNA, 18 bp, synthetic construct
  source 1..18: mol_type = other DNA
gatggggctctggtggcg

SEQ ID NO: 710 - protein, 176 aa, synthetic construct
SRPGERPFQCRICMRNFSRRQHLTLHTRTHTGEKPFQCRICMRNFSDNSHLQRHLRTHTGSQKPFQCRICMRNFSLPHHLQRHLRTHTGEKPFQCRICMRNFSQSNNLTRHLKTHTGSQKPFQCRICMRNFSRNFILQRHTRTHTGEKPFQCRICMRNFSRKDDLKRHLRTHLRGS

SEQ ID NO: 711 - DNA, 528 bp, synthetic construct
  source 1..528: mol_type = other DNA
tcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtct

SEQ ID NO: 712 - DNA, 18 bp, synthetic construct
  source 1..18: mol_type = other DNA
gcggtggaaggtggctgt

SEQ ID NO: 713 - protein, 1319 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSRTDTLARHLRTHTGEKPFQCRICMRNFSRKTALNRHLKTHTGSQKPFQCRICMRNFSRNESLKVHLRTHTGEKPFQCRICMRNFSVKNTLTRHLKTHTGSQKPFQCRICMRNFSRREHLVRHLRTHTGEKPFQCRICMRNFSISHNLARHLKTHLRGSGVDGSSGSETPGTSESATPESSRPGERPFQCRICMRNFSRRQHLTLHTRTHTGEKPFQCRICMRNFSDNSHLQRHLRTHTGSQKPFQCRICMRNFSLPHHLQRHLRTHTGEKPFQCRICMRNFSQSNNLTRHLKTHTGSQKPFQCRICMRNFSRNFILQRHTRTHTGEKPFQCRICMRNFSRKDDLKRHLRTHLRGSSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 714 - DNA, 1844 bp, synthetic construct
  source 1..1844: mol_type = other DNA
agctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcaagaacagacactctggcccgacacctgaggacccacactggagagaaaccctttcagtgcaggatatgtatgcggaatttttccagaaagaccgcacttaatcgacatctcaagacacataccgggagtcagaagcctttccaatgccggatttgcatgaggaacttctcccggaacgaatctctgaaggtgcacctgcgcacacatacaggcgagaagccattccagtgtaggatctgcatgcgcaattttagcgtgaagaatacactgacgagacatctgaagacgcatacaggttcccagaagccctttcagtgcaggatctgcatgaggaattttagtagaagggagcatctcgttagacacctccggacgcatactggagagaagccctttcagtgtaggatttgtatgcggaacttcagcataagccacaatctggcaaggcaccttaaaactcatttgcgcgggtctggtgttgatggctcctctggttctgaaactcctggtacttctgagtctgctactcccgaatcttcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtga

SEQ ID NO: 715 - DNA, 1430 bp, synthetic construct
  source 1..1430: mol_type = other DNA
tctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcaagaacagacactctggcccgacacctgaggacccacactggagagaaaccctttcagtgcaggatatgtatgcggaatttttccagaaagaccgcacttaatcgacatctcaagacacataccgggagtcagaagcctttccaatgccggatttgcatgaggaacttctcccggaacgaatctctgaaggtgcacctgcgcacacatacaggcgagaagccattccagtgtaggatctgcatgcgcaattttagcgtgaagaatacactgacgagacatctgaagacgcatacaggttcccagaagccctttcagtgcaggatctgcatgaggaattttagtagaagggagcatctcgttagacacctccggacgcatactggagagaagccctttcagtgtaggatttgtatgcggaacttcagcataagccacaatctggcaaggcaccttaaaactcatttgcgcgggtctggtgttgatggctcctctggttctgaaactcctggtacttctgagtctgctactcccgaatcttcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtctagccccaagaagaagagaaaggtgggagtcgacggatccagcggctccgagaccccaggcac

SEQ ID NO: 716 - protein, 1319 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSRRQHLTLHTRTHTGEKPFQCRICMRNFSDNSHLQRHLRTHTGSQKPFQCRICMRNFSLPHHLQRHLRTHTGEKPFQCRICMRNFSQSNNLTRHLKTHTGSQKPFQCRICMRNFSRNFILQRHTRTHTGEKPFQCRICMRNFSRKDDLKRHLRTHLRGSGVDGSSGSETPGTSESATPESSRPGERPFQCRICMRNFSRTDTLARHLRTHTGEKPFQCRICMRNFSRKTALNRHLKTHTGSQKPFQCRICMRNFSRNESLKVHLRTHTGEKPFQCRICMRNFSVKNTLTRHLKTHTGSQKPFQCRICMRNFSRREHLVRHLRTHTGEKPFQCRICMRNFSISHNLARHLKTHLRGSSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 717 - DNA, 1758 bp, synthetic construct
  source 1..1758: mol_type = other DNA
tctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtctggtgttgatggctcctctggttctgaaactcctggtacttctgagtctgctactcccgaatcttcccggccaggggaacggcccttccagtgtcggatctgcatgagaaacttttcaagaacagacactctggcccgacacctgaggacccacactggagagaaaccctttcagtgcaggatatgtatgcggaatttttccagaaagaccgcacttaatcgacatctcaagacacataccgggagtcagaagcctttccaatgccggatttgcatgaggaacttctcccggaacgaatctctgaaggtgcacctgcgcacacatacaggcgagaagccattccagtgtaggatctgcatgcgcaattttagcgtgaagaatacactgacgagacatctgaagacgcatacaggttcccagaagccctttcagtgcaggatctgcatgaggaattttagtagaagggagcatctcgttagacacctccggacgcatactggagagaagccctttcagtgtaggatttgtatgcggaacttcagcataagccacaatctggcaaggcaccttaaaactcatttgcgcgggtctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtga

SEQ ID NO: 718 - DNA, 1364 bp, synthetic construct
  source 1..1364: mol_type = other DNA
accagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtctggtgttgatggctcctctggttctgaaactcctggtacttctgagtctgctactcccgaatcttcccggccaggggaacggcccttccagtgtcggatctgcatgagaaacttttcaagaacagacactctggcccgacacctgaggacccacactggagagaaaccctttcagtgcaggatatgtatgcggaatttttccagaaagaccgcacttaatcgacatctcaagacacataccgggagtcagaagcctttccaatgccggatttgcatgaggaacttctcccggaacgaatctctgaaggtgcacctgcgcacacatacaggcgagaagccattccagtgtaggatctgcatgcgcaattttagcgtgaagaatacactgacgagacatctgaagacgcatacaggttcccagaagccctttcagtgcaggatctgcatgaggaattttagtagaagggagcatctcgttagacacctccggacgcatactggagagaagccctttcagtgtaggatttgtatgcggaacttcagcataagccacaatctggcaaggcaccttaaaactcatttgcgcgggtctagccccaagaagaagagaaaggtgggagtcgacggatccagcggctccgagaccccaggcac

SEQ ID NO: 719 - protein, 1322 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSGVDGSSGSETPGTSESATPESSRPGERPFQCRICMRNFSRRQHLTLHTRTHTGEKPFQCRICMRNFSDNSHLQRHLRTHTGSQKPFQCRICMRNFSLPHHLQRHLRTHTGEKPFQCRICMRNFSQSNNLTRHLKTHTGSQKPFQCRICMRNFSRNFILQRHTRTHTGEKPFQCRICMRNFSRKDDLKRHLRTHLRGSSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 720 - DNA, 1683 bp, synthetic construct
  source 1..1683: mol_type = other DNA
gagtctggacctggcaccagcacagagccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctggtgttgatggctcctctggttctgaaactcctggtacttctgagtctgctactcccgaatcttcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtga

SEQ ID NO: 721 - DNA, 1308 bp, synthetic construct
  source 1..1308: mol_type = other DNA
gccatccgagggctctgccccaggctctcctgcaggcagccctacctccaccgaagagggcaccagcacagagccttctgagggcagcgccccaggcacctctacagagccaagcgagtcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctggtgttgatggctcctctggttctgaaactcctggtacttctgagtctgctactcccgaatcttcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtctagccccaagaagaagagaaaggtgggagtcgacggatccagcggctccgagaccccaggcac

SEQ ID NO: 722 - protein, 1322 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSRRQHLTLHTRTHTGEKPFQCRICMRNFSDNSHLQRHLRTHTGSQKPFQCRICMRNFSLPHHLQRHLRTHTGEKPFQCRICMRNFSQSNNLTRHLKTHTGSQKPFQCRICMRNFSRNFILQRHTRTHTGEKPFQCRICMRNFSRKDDLKRHLRTHLRGSGVDGSSGSETPGTSESATPESSRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 723 - DNA, 1599 bp, synthetic construct
  source 1..1599: mol_type = other DNA
ggcaccagcacagagccttctgagggcagcgccccaggcacctctacagagccaagcgagtcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtctggtgttgatggctcctctggttctgaaactcctggtacttctgagtctgctactcccgaatcttcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtga

SEQ ID NO: 724 - DNA, 1243 bp, synthetic construct
  source 1..1243: mol_type = other DNA
gcacagagccttctgagggcagcgccccaggcacctctacagagccaagcgagtcccggccaggggaacggcccttccagtgtcggatctgcatgagaaacttttcacgcagacagcacctgacactccacactcgaacccacactggagagaaaccctttcagtgcaggatatgtatgcggaatttttccgacaacagccacctgcaaaggcatctccgcacacataccgggagtcagaagcctttccaatgccggatttgcatgaggaacttctcccttccccaccatctgcagcgccacctgcggacacatacaggcgagaagccattccagtgtaggatctgcatgcgcaattttagccagtctaataaccttactcgccacctcaagacgcatacaggttcccagaagccctttcagtgcaggatctgcatgaggaattttagtaggaacttcatactgcaacggcacacaagaacgcatactggagagaagccctttcagtgtaggatttgtatgcggaacttcagcaggaaggacgatctgaaaagacatctgagaactcatttgcgcgggtctggtgttgatggctcctctggttctgaaactcctggtacttctgagtctgctactcccgaatcttcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagccccaagaagaagagaaaggtgggagtcgacggatccagcggctccgagaccccaggcac

SEQ ID NO: 725 - protein, 1325 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSGVDGSSGSETPGTSESATPESSRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 726 - DNA, 1439 bp, synthetic construct
  source 1..1439: mol_type = other DNA
agtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctggtgttgatggctcctctggttctgaaactcctggtacttctgagtctgctactcccgaatcttcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtga

SEQ ID NO: 727 - DNA, 1120 bp, synthetic construct
  source 1..1120: mol_type = other DNA
cacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctggtgttgatggctcctctggttctgaaactcctggtacttctgagtctgctactcccgaatcttcccggccaggggaacggcccttccagtgtcggatctgcatgagaaacttttcaacaccctcaaagctggataggcatacacgcacccacactggagagaaaccctttcagtgcaggatatgtatgcggaatttttccctggctgagaacctccgaaggcacctgagaacacataccgggagtcagaagcctttccaatgccggatttgcatgaggaacttctcccgccaagataacctgggccgccacctgcgcacacatacaggcgagaagccattccagtgtaggatctgcatgcgcaattttagcgacggcggtaacttgggacgccacctcaaaacgcatacaggtggcggtggctcccagaagccctttcagtgcaggatctgcatgaggaattttagtcagtccccacatcttaaaagacacttgcggacgcatactggagagaagccctttcagtgtaggatttgtatgcggaacttcagccagagcacaagcctgcaacgccatctcaaaactcatttgcgcgggtctagccccaagaagaagagaaaggtgggagtcgacggatccagcggctccgagaccccaggcac

(19) United States
(12) Patent Application Publication
(10) Pub. No.: US 2026/0286319 A1
(43) Pub. Date: Sep. 24, 2026

(54) FUSION PROTEINS FOR EPIGENETIC REGULATION

(71) Applicant: nChroma Bio, Inc., Boston, MA (US)

(72) Inventors: Shane Howard Wright, Boston, MA (US); Vic Myer, Arlington, MA (US); Morgan Maeder, Waban, MA (US)

(73) Assignee: nChroma Bio, Inc., Boston, MA (US)

(21) Appl. No.: 19/476,668

(22) PCT Filed: Apr. 19, 2024
(86) PCT No.: PCT/US2024/025483

§ 371 Date: Oct. 17, 2025

Related U.S. Application Data
(60) Provisional application No. 63/460,829, filed on Apr. 20, 2023.

(51) Int. Cl. C12N 9/10; A61K 38/00; C07K 14/47

(52) U.S. Cl. CPC C12N 9/1007; C07K 14/4703; C12Y 201/01037; A61K 38/00; C07K 2319/02

(57) ABSTRACT

This disclosure provides compositions and methods comprising epigenetic editors comprising two or more DNA-binding zinc finger domains for epigenetic modification of target genes, as well as nucleic acids and vectors encoding the same. Also disclosed are cells epigenetically modified by the epigenetic editors.

### CROSS-REFERENCE TO RELATED APPLICATIONS

This application is a national stage filing under 35 U.S.C. § 371 of international PCT application PCT/US2024/025483, filed Apr. 19, 2024, entitled “FUSION PROTEINS FOR EPIGENETIC REGULATION”, which claims the benefit under 35 U.S.C. § 119(e) of U.S. Provisional Application No. 63/460,829, filed Apr. 20, 2023, entitled “FUSION PROTEINS FOR EPIGENETIC REGULATION,” the contents of each of which are hereby incorporated by reference in their entirety.

### REFERENCE TO AN ELECTRONIC SEQUENCE LISTING

The contents of the electronic sequence listing (C169870001US01-SUBSEQ-AXW.xml; Size: 852,262 bytes; and Date of Creation: Nov. 3, 2025) are herein incorporated by reference in their entirety.

### BACKGROUND

Genome editing has been considered a promising therapeutic approach for the treatment of genetic disease for over a decade. However, manipulation on the DNA level using traditional genetic editors remains risky given the potential for undesired double-strand DNA breaks, heterogenous repair (including large and small insertions and deletions at the intended site), chromosomal translocations, and toxicity. By contrast, targeted epigenetic modification offers the potential to alter gene expression without inducing genomic mutations and the associated risks.

### SUMMARY

The present disclosure provides systems and compositions for epigenetic modification (“epigenetic editors” or “epigenetic editing systems” herein), and methods of using the same to generate epigenetic modification of target genes, including in host cells and organisms.

Some aspects of this disclosure provide engineered, i.e., non-naturally occurring, fusion proteins comprising a DNA-binding domain, which comprises a plurality of DNA-binding zinc finger domains, and one or more effector domains. In some embodiments, the DNA-binding domains comprises two DNA-binding zinc finger domains. In some embodiments, the DNA-binding domains comprises three DNA-binding zinc finger domains. In some embodiments, the DNA-binding domains comprises four or more DNA-binding zinc finger domains. In some embodiments, each DNA-binding zinc finger domain comprised in the fusion protein comprises four, five, six, or seven individual zinc finger sequences. In some embodiments, each DNA-binding zinc finger domain comprised in the fusion protein specifically binds a target DNA sequence in a genome, e.g., in a human genome. In some embodiments, the target DNA sequence bound by the different DNA-binding zinc finger domains is comprised in the same gene of the genome. In some embodiments, the target DNA sequence bound by the different DNA-binding zinc finger domains is comprised in different genes of the genome. In some embodiments, the effector domain is a DNA-modifying enzyme domain (e.g., a DNA methyltransferase domain; a methylcytosine dioxygenase domain, a deaminase domain, such as, for example, a cytosine or adenosine deaminase domain, an endonuclease domain, a nickase domain, a transposase domain, or a recombinase domain); a histone-modifying enzyme domain (e.g., a histone methyltransferase domain, a histone methylase domain, a histone deacetylase domain, a histone acetyltransferase domain); a chromatin remodeling domain; a transcriptional activator domain, or a transcriptional repressor domain.

In some aspects, the present disclosure provides a fusion protein comprising one or more DNA methyltransferase (DNMT) domains and/or a recruiter domain that recruits a DNMT, two or more DNA-binding zinc finger domains, and one or more transcriptional repressor domain. In some embodiments, the fusion protein comprises, from N-terminus to C-terminus, a DNMT domain and/or a recruiter domain that recruits a DNMT, a first DNA-binding zinc finger domain, a second DNA-binding zinc finger domain, and a transcriptional repressor domain. In some embodiments, the fusion protein comprises, from N-terminus to C-terminus, a transcriptional repressor domain, a first DNA-binding zinc finger domain, a second zinc finger domain, and a DNMT domain and/or a recruiter domain that recruits a DNMT. In some embodiments, the fusion protein comprises, from N-terminus to C-terminus, a DNMT domain and/or a recruiter domain that recruits a DNMT, a first DNA-binding zinc finger domain, a transcriptional repressor domain, and a second DNA-binding zinc finger domain. In some embodiments, the fusion protein comprises, from N-terminus to C-terminus, a DNA-binding zinc finger domain, a transcriptional repressor domain, a second DNA-binding zinc-finger domain, and a DNMT domain and/or a recruiter domain that recruits a DNMT. In some embodiments, the fusion protein comprises, from N-terminus to C-terminus, a first transcriptional repressor domain, a first DNA-binding zinc finger domain, a DNMT domain and/or a recruiter domain that recruits a DNMT, a second DNA-binding zinc finger domain, and a second transcriptional repressor domain.

In some embodiments, at least one of the transcriptional repressor domains comprises a KRAB domain. In some embodiments, at least one of the transcriptional repressor domains comprises a KRAB domain derived from KOX1. In some embodiments, at least one of the transcriptional repressor domains comprises a KRAB domain derived from ZIM3. In some embodiments, at least one of the transcriptional repressor domains comprises a KRAB domain derived from ZFP28. In some embodiments, at least one of the transcriptional repressor domains comprises a KRAB domain derived from ZN627. In some embodiments, the KRAB domain comprises a sequence with at least at least 75%, 80%, 85%, 90%, 91%, 92%, 93%, 94%, 95%, 96%, 97%, 98%, or 99% identity to SEQ ID NO: 89. In some embodiments, the KRAB domain comprises a sequence with at least 75%, 80%, 85%, 90%, 91%, 92%, 93%, 94%, 95%, 96%, 97%, 98%, or 99% identity to SEQ ID NO: 116. In some embodiments, the KRAB domain comprises a sequence with at least 75%, 80%, 85%, 90%, 91%, 92%, 93%, 94%, 95%, 96%, 97%, 98%, or 99% identity to SEQ ID NO: 245. In some embodiments, the KRAB domain comprises a sequence with at least 75%, 80%, 85%, 90%, 91%, 92%, 93%, 94%, 95%, 96%, 97%, 98%, or 99% identity to SEQ ID NO: 255. In some embodiments, at least one of the transcriptional repressor domains comprises a fusion of the N- and C-terminal regions of ZIM3 and KOX1 KRAB, and optionally comprises the amino acid sequence of SEQ ID NO: 571 or 572. In some embodiments, at least one of the transcriptional repressor domains is derived from KAP1, MECP2, HP1a/CBX5, HP1b, CBX8, CDYL2, TOX, TOX3, TOX4, EED, EZH2, RBBP4, RCOR1, or SCML2.

In some embodiments, the DNMT domain and/or the recruiter domain that recruits a DNMT comprises a DNMT3A domain and/or a DNMT3L domain, optionally wherein the recruited DNMT is a DNMT3A.

In some embodiments, the fusion comprises SEQ ID NO: 658 or a sequence at least 75%, 80%, 85%, 90%, 91%, 92%, 93%, 94%, 95%, 96%, 97%, 98%, or 99% identical thereto. In some embodiments, the fusion comprises SEQ ID NO: 23 or a at least 75%, 80%, 85%, 90%, 91%, 92%, 93%, 94%, 95%, 96%, 97%, 98%, or 99% identical thereto. In some embodiments, the fusion comprises SEQ ID NO: 659 or a sequence at least at least 75%, 80%, 85%, 90%, 91%, 92%, 93%, 94%, 95%, 96%, 97%, 98%, or 99% identical thereto.

In some embodiments, the first DNA-binding zinc finger domain and the second DNA-binding zinc finger domain are the same. In some embodiments, the first DNA-binding zinc finger domain and the second DNA-binding zinc finger domain bind the same target nucleic acid sequence. In some embodiments, the first DNA-binding zinc finger domain and the second DNA-binding zinc finger domain are different. In some embodiments, the first DNA-binding zinc finger domain and the second DNA-binding zinc finger domain bind different target nucleic acid sequences.

In some embodiments, the fusion protein further comprises a third DNA-binding zinc finger domain. In some embodiments, the fusion protein further comprises at least one additional DNA-binding zinc finger domain.

In some embodiments, the first DNA-binding zinc finger domain, the second DNA-binding zinc finger domain, and the third DNA-binding zinc finger domain are the same. In some embodiments, the first DNA-binding zinc finger domain and the second DNA-binding zinc finger domain are the same. In some embodiments, the first DNA-binding zinc finger domain and the third DNA-binding zinc finger domain are the same. In some embodiments, the second DNA-binding zinc finger domain and the third DNA-binding zinc finger domain are the same. In some embodiments, each of the DNA-binding zinc finger domains is a different DNA-binding zinc finger domain.

In some embodiments, the fusion protein targets the same genetic element. In some embodiments, the fusion protein targets multiple sites in the same genetic element. In some embodiments, the fusion protein targets two or more genetic elements.

In some embodiments, the fusion protein increases DNA accessibility or availability for editing.

In some embodiments, the fusion protein further comprises one or more linkers See e.g., Schellenberger et al., Nat. Biotechnol., 27: 1186-1190 (2009). In some embodiments, the one or more linkers comprise an unstructured amino acid sequence, e.g., an amino acid sequence that does not form a specific secondary structure. In some embodiments, the one or more linkers comprise a flexible amino acid sequence, e.g., an amino acid sequence that does not form a specific secondary structure, such as, for example, an alpha helix or a beta sheet. In some embodiments, the one or more linkers comprise an XTEN sequence. In some embodiments, the one or more linkers comprise an XTEN80 and/or an XTEN16 linker sequence.

In some embodiments, the fusion protein further comprises one or more nuclear localization signals (NLS). In some embodiments, at least one of the NLSs is an SV40 NLS.

In some embodiments, the fusion protein comprises from N-terminus to C-terminus, a DNMT domain and/or a recruiter domain that recruits a DNMT, a first DNA-binding zinc finger domain, a second DNA-binding zinc finger domain, and a transcriptional repressor domain.

In some embodiments, the fusion protein comprises from N-terminus to C-terminus, a transcriptional repressor domain, a first DNA-binding zinc finger domain, a second DNA-binding zinc finger domain, and a DNMT domain and/or a recruiter domain that recruits a DNMT.

In some embodiments, the fusion protein comprises a linker sequence connecting the DNMT domain and/or the recruiter domain to the adjacent DNA-binding zinc finger domain. In some embodiments, the fusion protein comprises a linker sequence connecting the transcriptional repressor domain to the adjacent DNA-binding zinc finger domain. In some embodiments, at least one of the linkers connecting the DNMT domain and/or the recruiter domain to the adjacent DNA-binding zinc finger domain or connecting the transcriptional repressor domain to the adjacent DNA-binding zinc finger domain comprises an XTEN linker, e.g., an XTEN80 linker or an XTEN16 linker.

In some embodiments, the fusion protein comprises from N-terminus to C-terminus, two nuclear localization signals (NLSs), a DNMT domain and/or a recruiter domain that recruits a DNMT, a first linker, a first DNA-binding zinc finger domain, a second linker, a second DNA-binding zinc finger domain, a third NLS, a third linker, a transcriptional repressor domain, and two NLSs.

In some embodiments, the fusion protein comprises, from N-terminus to C-terminus, two nuclear localization signals (NLSs), a transcriptional repressor domain, a first linker, a third NLS, a first DNA-binding zinc finger domain, a second linker, a second DNA-binding zinc finger domain, a third linker, a DNMT domain and/or a recruiter domain that recruits a DNMT, and two NLSs.

In some embodiments, the fusion protein comprises, from N-terminus to C-terminus, two nuclear localization signals, a transcriptional repressor domain, a first linker, a first DNA-binding zinc finger domain, a second linker, a DNMT domain and/or a recruiter domain that recruits a DNMT, a third linker, a third NLS, a second DNA-binding zinc finger domain, a fourth linker, a second transcriptional repressor domain, and two additional NLSs.

In some embodiments, the fusion protein comprises, from N-terminus to C-terminus, two nuclear localization signals (NLSs), a transcriptional repressor domain, a first linker, a first DNA-binding zinc finger domain, a third NLS, a second linker, a DNMT domain and/or a recruiter domain that recruits a DNMT, a third linker, a second DNA-binding zinc finger domain, a fourth linker, a second transcriptional repressor domain, and two NLSs.

The present disclosure provides systems and compositions for epigenetic modification (“epigenetic editors” or “epigenetic editing systems” herein), and methods of using the same to generate epigenetic modification of a target gene or a plurality of target genes, including in host cells, e.g., in vitro or ex vivo, and organisms, e.g., in vivo.

In some embodiments, at least one of the linkers comprises an XTEN linker, optionally an XTEN80 linker or an XTEN16 linker.

Some aspects of this disclosure provide a nucleic acid sequence encoding a fusion protein provided herein. In some embodiments, such a nucleic acid sequence comprises one or more regulatory elements, e.g., a 5′ untranslated region (UTR) and/or a 3′UTR. In some embodiments, such a nucleic acid sequence comprises a post-transcriptional regulatory element, for example, a woodchuck hepatitis virus post-transcriptional regulatory element (WPRE).

In some aspects, the present disclosure provides a composition comprising a fusion protein provided herein, or a nucleic acid encoding such a fusion protein, e.g., an mRNA, formulated for administration to a subject in need thereof, e.g., a human subject in need thereof. For example, in some aspects, the present disclosure provides a composition comprising a fusion protein provided herein, or a nucleic acid encoding such a fusion protein, e.g., an mRNA, wherein the fusion protein or the encoding nucleic acid is formulated into a lipid nanoparticle (LNP). For example, in some aspects, the present disclosure provides a composition comprising a nucleic acid encoding such a fusion protein, e.g., an encoding DNA or RNA, wherein the encoding nucleic acid is comprised in a viral vector, e.g., an AAV vector.

In some aspects, the present disclosure provides a cell comprising a fusion protein of the present disclosure or a nucleic acid of the present disclosure, or progeny of the cell. In some embodiments, the cell is a human cell.

In some aspects, the present disclosure provides a composition comprising a fusion protein of the present disclosure, a nucleic acid of the present disclosure, or a cell of the present disclosure. In some embodiments, the composition further comprises a pharmaceutically acceptable excipient.

In some aspects, the present disclosure provides a method comprising administering to a subject a fusion protein of the present disclosure, a nucleic acid of the present disclosure, a cell of the present disclosure, or a pharmaceutical composition of the present disclosure. In some embodiments, the subject is human.

In some aspects, the present disclosure provides a system for modulating transcription of one or more genes, e.g., one or more human genes, in a cell, e.g., in a human cell, comprising
i) a fusion protein of the present disclosure, or
ii) a nucleic acid of the present disclosure.

In some embodiments, the system modulates transcription of a plurality of genes, e.g., a plurality of human genes, in a cell, e.g., in a human cell, or in a subject in need thereof, e.g., in a human subject in need thereof. In some embodiment, the modulating is silencing (reducing the expression level) of one or more genes in a cell or subject, e.g., by at least 50%, at least 60%, at least 70%, at least 75%, at least 80%, at least 90%, at least 95%, at least 99%, at least 99.9%, as compared to a control expression level, e.g., an expression level measured or expected in an untreated cell or subject. In some embodiments, the fusion protein increases DNA accessibility or availability for editing. In some embodiments, the system causes durable silencing of one or more human genes, e.g., over a time period of at least 7 days, at least 14 days, at least 21 days, at least 28 days, at least 60 days, at least 90 days, at least 180 days, at least 240 days, at least 360 days, or longer. In some embodiments, the system increases transcription of one or more human genes.

### BRIEF DESCRIPTION OF DRAWINGS

FIG. 1 shows a diagram illustrating the requirement for a plurality of polynucleotide constructs (either encoding separate zinc finger-based epigenetic editors (top left) or a single CRISPR-Off type epigenetic editor with multiple guide RNAs) for multiplex targeting and the implication on exemplary restrictions on total amount of polynucleotide that can be administered at once.

FIG. 2 shows exemplary conformations of epigenetic editors of the present disclosure, comprising two DNA-binding zinc finger domains. Illustrated in this figure are two exemplary conformations for multiplex targeting using two different, adjacent DNA-binding zinc finger domains (Top: Dnmt3A-Dnmt3L-DNA-binding zinc finger domain 1-DNA-binding zinc finger domain 2-KRAB; Middle: Dnmt3A-Dnmt3L-DNA-binding zinc finger domain 2-DNA-binding zinc finger domain 1-KRAB). Further illustrated is a palindromic fusion protein of the structure KRAB-DNA-binding zinc finger domain 1-Dnmt3A-Dnmt3L-DNA-binding zinc finger domain 2-KRAB.

FIGS. 3A-3E show graphs demonstrating gene silencing by exemplary epigenetic editors of the present disclosure. FIGS. 3A and 3B show day 7 silencing of PCSK9 as measured by FACS in a HeLa cell line harboring a TdTomato sequence insertion at the endogenous PCSK9 genomic locus. FIGS. 3C and 3D show day 7 silencing of CLTA as measured by FACS in a GripTite®-293 cell line harboring a GFP sequence insertion at the endogenous CLTA genomic locus. FIG. 3E shows a summary of silencing at both loci in both PCSK9:TdTomato HeLa cells and CLTA:GFP Griptite-293 cells. For each condition, two bars are shown. The left bar shows silencing on PCSK9 HeLa cells; the right bar shows silencing on CLTA Griptites. All data is displayed as the percentage of cells expressing the given fluorophore. CRISPRi PCSK9: CRISPRi construct with PCSK9 gRNA; CRISPR-OFF PCSK9: CRISPR-OFF construct with PCSK9 gRNA; single ZF-OFF PCSK9: ZF-OFF construct with ZF152 PCSK9 DNA-binding zinc finger domain; single ZF-OFF CLTA:ZF-OFF construct with ZF2244 CLTA DNA-binding zinc finger domain; Split Dose: ZF-OFF PCSK9 and ZF-OFF CLTA each at half the dose of the single ZF-OFFs; PCSK9-CLTA: dual targeting PCSK9/CLTA ZF-OFF construct-Conformation 1; CLTA-PCSK9: dual targeting CLTA/PCSK9 ZF-OFF construct-Conformation 2; R-CLTA-3A/3L-PCSK9-R: dual-targeting, palindromic CLTA/PCSK9 ZF-off construct-Conformation 3.

FIGS. 4A-4C show silencing of target genes over time. FIG. 4A shows silencing of PCSK9 over 17 days by exemplary constructs described herein. FIG. 4B shows silencing of CLTA over 17 days by exemplary constructs described herein. FIG. 4C shows silencing of PCSK9 and CLTA for more than 15 days by a single exemplary construct described herein (PCSK9-CLTA).

FIGS. 5A-5E shows plots of gene expression as measured by RNAseq in PCSK9:TdTomato HeLa cells at 14 days post treatment. Expression of genes in PCSK9:TdTomato HeLa cells treated with an epigenetic editor described herein was measured by RNAseq and compared to expression in an untreated cells. The number of differentially expressed (DE) genes indicates the extent of possible off-target or downstream effects of silencing by each construct. FIG. 5A shows differential expression by a control CRISPR-OFF construct FIG. 5B shows differential expression by the single PCSK9 ZF-OFF (ZF-OFF construct with ZF152 PCSK9 DNA-binding zinc finger domain) at a 50 ng dose. FIG. 5C shows differential expression by the single CLTA ZF-OFF construct (ZF-OFF construct with ZF2244 CLTA DNA-binding zinc finger domain) at a 50 ng dose. FIG. 5B-C illustrate the off-target profile of each zinc finger contained within the exemplary construct described herein (PCSK9-CLTA: dual targeting PCSK9/CLTA ZF-OFF construct-Conformation 1) FIG. 5D shows differential expression by cells treated with a single 50 ng dose containing two single zinc finger constructs, wherein one construct targets CLTA (ZF-OFF construct with ZF2244 CLTA DNA-binding zinc finger domain) and the other construct targets PCSK9 (ZF-OFF construct with ZF152 PCSK9 DNA-binding zinc finger domain) in a 1:1 ratio. The number and identity of the differentially expressed genes is similar to the combination of the RNAseq profiles for each construct (FIG. 5B-C). FIG. 5E shows differential expression of PCSK9:TdTomato cells treated with a 50 ng dose of a single epigenetic editor comprising two DNA-binding zinc finger domains (PLA4095dual targeting PCSK9/CLTA ZF-OFF construct-Conformation 1 ZFP152-XTEN16-ZFP2244). The RNAseq profile looks comparable to that of cells treated with two distinct zinc finger constructs (FIG. 5D) indicating that that combining two or more DNA-binding zinc finger domains in a single epigenetic editor does not result in significant variations in specificity as compared to the specificity profiles of a combination of a plurality of epigenetic editors each targeting a single target.

FIG. 6 shows a schematic of the construct design of traditional “zinc finger” silencer and “zinc hands” silencers.

FIG. 7 shows a schematic of a dual reporter system.

FIG. 8 shows plots of flow cytometry of silencer targets for cells with dual reporter system after transfection with dual-targeting silencers or a control consisting of two individual single zinc finger epigenetic silencers (“Split Dose”)-one targeting PCSK9 and the other targeting CLTA. Cells shown were transfected with 50 ng total mRNA.

FIG. 9 is a line graph of dose response for dual-targeting epigenetic silencers in a dual fluorescent reporter HeLa cell line at day 14. % double negative cells is graphed as a function of dose. n=2.

FIG. 10 shows the name, relative silencing efficacy as compared to ZF #2, and target position (“locus”) within PCSK9. The composition of “adjacent” and “distal” zinc hand constructs is indicated.

FIG. 11 is a schematic of the spatial orientation of an ordered zinc finger/DNA complex and a disordered zinc finger/DNA complex.

FIGS. 12A-12C show line graphs of dose response for distal (FIG. 12A), adjacent (FIG. 12B), and redundant (FIG. 12C) zinc hands targeting PCSK9. PCSK9 expression is graphed as a function of dose. n=1. “CRISPR-OFF” serves as a positive control for silencing and comprises a Cas9 protein with intact endonuclease activity. “LO” indicates “lipid only” and serves as a negative control for silencing.

### DETAILED DESCRIPTION

In some aspects, the present disclosure provides fusion proteins comprising a plurality of DNA-binding zinc finger domains and at least one effector domain. DNA-binding zinc finger domains are known to provide a flexible option for binding to many diverse DNA sequences. While these domains have been used for targeting effector domains to a particular genetic element, fusion proteins for genetic editing comprising two or more DNA-binding zinc finger domains have not been previously identified. The fusion proteins provided herein are useful for efficient targeting of effector domains to specific sequences within a genome, e.g., wherein each DNA-binding zinc finger domain of a given fusion protein binds a different target sequence within a genome, e.g., a genome of a cell or a subject, thus allowing multiplex targeting with a single protein and decreasing the manufacturing burden for scaling up production of the therapy. The fusion proteins provided herein are further useful for multiplex genetic or epigenetic editing, e.g., for silencing or activating one or more target genes within a genome.

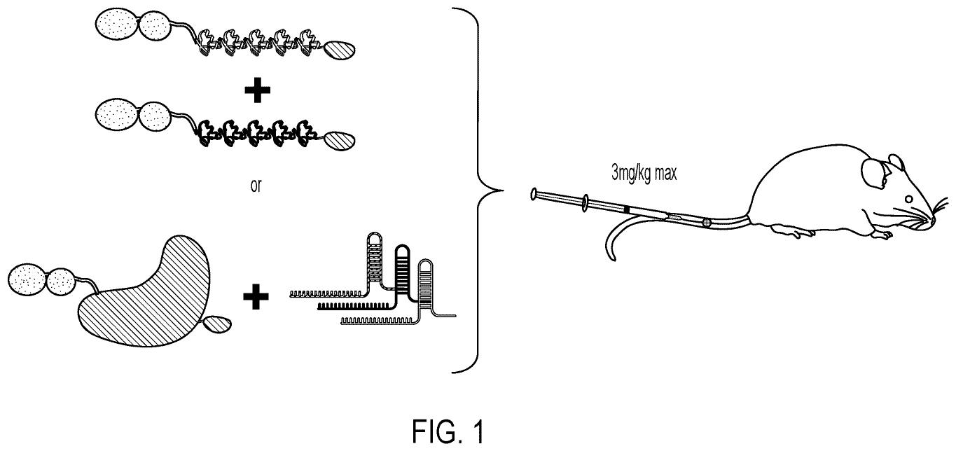

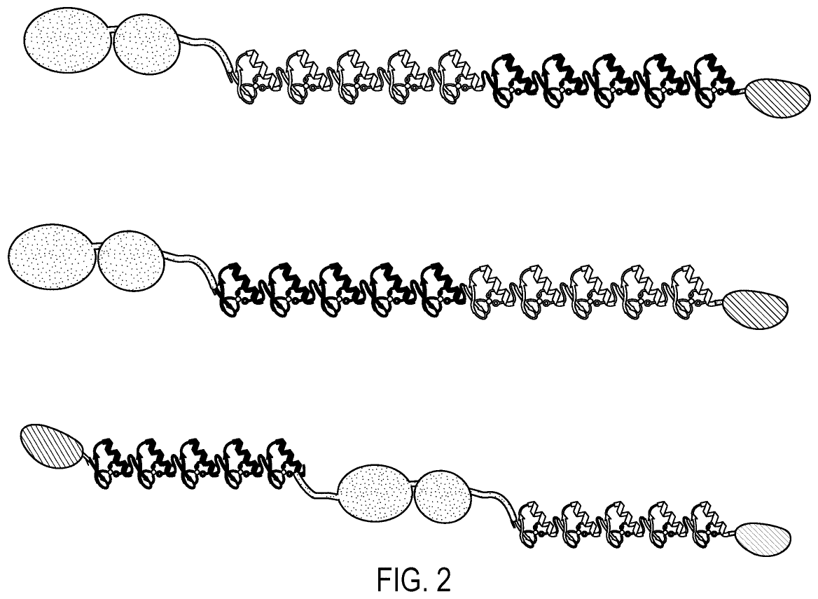

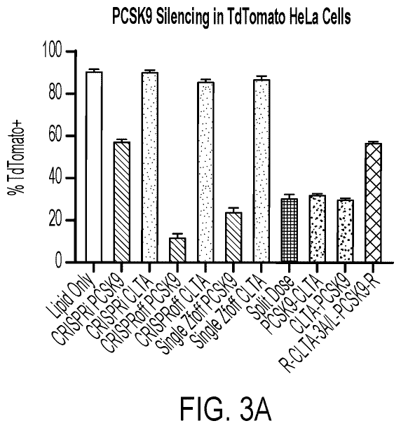

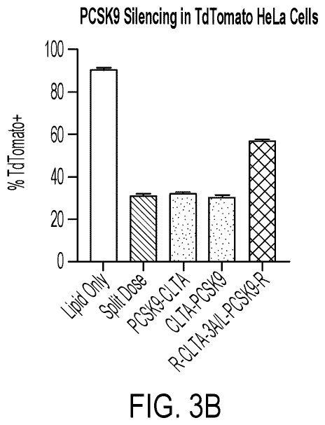

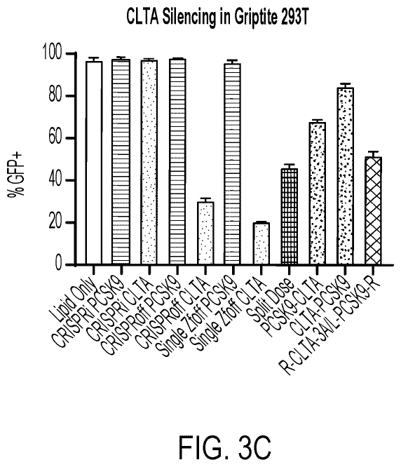

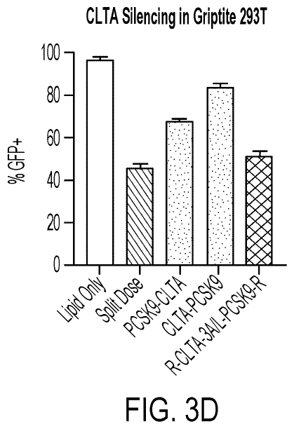

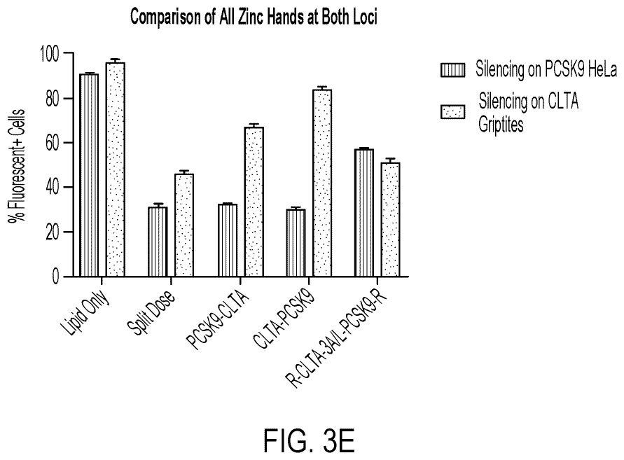

The fusion proteins comprising two or more DNA-binding zinc finger domains provided herein present several benefits. For example, fusion proteins comprising a plurality of DNA-binding zinc finger domains, wherein each zinc finger domain binds a different target sequence within a genome, allow multiplex targeting with a single, relatively small protein (see FIG. 2) instead of using a plurality of proteins binding only a single target sequence (see FIG. 1), thus addressing restrictions of payload size and/or delivered dose of some administration methods, reducing the number of administrations or deliveries to a target cell or subject, and simplifying manufacturing and formulation processes.

### I. DNA-Binding Domains

In some embodiments, a fusion protein, e.g., an epigenetic editor, described herein comprises two or more DNA-binding domains that direct the effector domain(s) of the fusion protein to target sequences within or close to a target gene locus. Fusion proteins of the present disclosure comprise two or more DNA-binding domain comprising DNA-binding zinc finger domains. Fusion proteins disclosed herein may comprise one or more additional DNA-binding domains, e.g., a polynucleotide guided DNA-binding domain, a third zinc finger protein (ZFP) DNA-binding domain, a transcription activator like effector (TALE) domain, a meganuclease DNA-binding domain, and the like. Examples of DNA-binding domains can be found in U.S. Pat. No. 11,162,114, which is incorporated by refence herein in its entirety.

In some embodiments, a DNA-binding domain described herein is encoded by its native coding sequence. In other embodiments, the DNA-binding domain is encoded by a nucleotide sequence that has been codon-optimized for optimal expression in human cells.

### A. Zinc Finger Protein Domains

In some aspects, the present disclosure provides fusion proteins e.g., epigenetic editors, comprising two or more DNA-binding domains comprising zinc finger protein (ZFP) domains (“ZFP domain” as used herein). ZFPs are proteins having at least one zinc finger and bind to DNA in a sequence-specific manner. A “zinc finger” (ZF) or “zinc finger motif” (ZF motif) refers to a polypeptide domain comprising a beta-beta-alpha (00a)-protein fold stabilized by a zinc ion. A ZF binds from two to four base pairs of nucleotides, typically three or four base pairs (contiguous or noncontiguous). Each ZF typically comprises approximately 30 amino acids. ZFP domains may contain multiple ZFs that make tandem contacts with their target nucleic acid sequence. A tandem array of ZFs may be engineered to generate artificial ZFPs that bind desired nucleic acid targets. ZFPs may be rationally designed by using databases comprising triplet (or quadruplet) nucleotide sequences and individual ZF amino acid sequences, in which each triplet or quadruplet nucleotide sequence is associated with one or more amino acid sequences of ZFs that bind the particular triplet or quadruplet sequence. See, e.g., U.S. Pat. Nos. 6,453,242, 6,534,261, and 8,772,453.

ZFPs are widespread in eukaryotic cells, and may belong to, e.g., C2H2 class, CCHC class, PHD class, or RING class. An exemplary motif characterizing one class of these proteins (C2H2 class) is -Cys-(X)₂₋₄-Cys-(X)₁₂-His-(X)₃₋₅-His- (SEQ ID NO: 657), where X is any independently chosen amino acid. In some embodiments, a ZFP domain herein may comprise a ZF array comprising sequential C2H2-ZFs each contacting three or more sequential nucleotides.

An individual ZFP domain of a fusion protein, e.g., of an epigenetic editor, described herein may include 2, 3, 4, 5, 6, 7, 8, 9, 10, 11, 12, or more ZFs. An individual ZFP domain may include an array of two-finger or three-finger units, e.g., 3, 4, 5, 6, 7, 8, 9 or 10 or more units, wherein each unit binds a subsite in the target sequence. In some embodiments, an individual ZFP domain comprising at least three ZFs recognizes a target DNA sequence of 9 or 10 nucleotides. In some embodiments, an individual ZFP domain comprising at least four ZFs recognizes a target DNA sequence of 12 to 14 nucleotides. In some embodiments, an individual ZFP domain comprising at least six ZFs recognizes a target DNA sequence of 18 to 21 nucleotides. In some embodiment, a fusion protein provided herein, comprises two or more (e.g., 2, 3, 4, or 5) individual ZFP domains.

In some embodiments, ZFs in a ZFP domain described herein are connected via peptide linkers. The peptide linkers may be, e.g., 2, 3, 4, 5, 6, 7, 8, 9, 10, 11, 12, 13, 14, 15, 16, 17, 18, 19, 20, or more amino acids in length. In some embodiments, a linker comprises 5 or more amino acids. In some embodiments, a linker comprises 7-17 amino acids. The linker may be flexible or rigid.

The design of individual, polydactyl DNA-binding zinc finger domains is well known in the art. Suitable peptide linkers, design criteria, and design strategies for generating polydactyl DNA-binding zinc finger domains specifically binding a target nucleic acid sequence as embraced by the present disclosure include the linkers, design criteria, and design strategies disclosed in Gersbach et al., Synthetic zinc finger proteins: the advent of targeted gene regulation and genome modification technologies. Acc Chem Res. (2014) 47(8):2309-18; Tan et al., Zinc-finger protein-targeted gene regulation: genomewide single-gene specificity. Proc Natl Acad Sci USA. (2003) 100(21):11997-2002; Miller et al., An improved zinc-finger nuclease architecture for highly specific genome editing. Nat Biotechnol. (2007) 25(7):778-85; Urnov et al., Highly efficient endogenous human gene correction using designed zinc-finger nucleases. Nature. (2005) 435(7042):646-51; Urnov et al., Genome editing with engineered zinc finger nucleases. Nat Rev Genet. (2010) 11(9):636-46; Negi et al., The past, present, and future of artificial zinc finger proteins: design strategies and chemical and biological applications. J Biol Inorg Chem (2023) 28:249-261; the entire contents of each of which are incorporated herein by reference.

In some exemplary embodiments a zinc finger array (an individual DNA-binding zinc finger domain) comprising six zinc fingers may have the sequence:

(SEQ ID NO: 650)
SRPGERPFQCRICMRNFSXXXXXXXHXXTHTGEKPFQCRICMRNF
SXXXXXXXHXXTH[linker]FQCRICMRNFSXXXXXXXHXXTHT
GEKPFQCRICMRNFSXXXXXXXHXXTH[linker]PFQCRICMRN
FSXXXXXXXHXXTHTGEKPFQCRICMRNFSXXXXXXXHXXTHLRG
S,

or a sequence at least 70%, 75%, 80%, 85%, 90%, 95%, 96%, 97%, 98%, or 99% identical thereto, where “XXXXXXX” represents the amino acids of the ZF recognition helix, which confers DNA-binding specificity upon the zinc finger; each X may be independently chosen. In the above sequence, “XX” in italics may be TR, LR or LK, and “[linker]” represents a linker sequence. In some embodiments, the linker sequence is TGSQKP (SEQ ID NO: 651); this linker may be used when sub-sites targeted by the ZFs are adjacent. In some embodiments, the linker sequence is TGGGGSQKP (SEQ ID NO: 652); this linker may be used when there is a base between the sub-sites targeted by the zinc fingers. The two indicated linkers may be the same or different.

Fusion proteins disclosed herein comprising two or more DNA-binding ZFP domains may contain arrays of two or more adjacent ZFs that are directly adjacent to one another (e.g., separated by a short (canonical) linker sequence), or are separated by longer, flexible or structured polypeptide sequences. In some embodiments, directly adjacent zinc fingers bind to contiguous nucleic acid sequences, i.e., to adjacent trinucleotides/triplets. In some embodiments, adjacent fingers cross-bind between each other's respective target triplets, which may help to strengthen or enhance the recognition of the target sequence, and lead to the binding of overlapping sequences. In some embodiments, distant ZFs within the ZFP domain may recognize (or bind to) non-contiguous nucleotide sequences.

In some embodiments, the first and second DNA-binding zinc finger domains of a fusion protein disclosed herein are the same. In some embodiments, the first and second DNA-binding zinc finger domains of a fusion protein disclosed herein are different. In some embodiments, the first and second DNA-binding zinc finger domains target multiple sites in the same genetic element, e.g., in the same promoter of a gene, in the same regulatory region of a gene, or in the same coding sequence of a gene. In some embodiments, the first and second DNA-binding zinc finger domains target different genetic elements, e.g., different genetic elements of the same gene (for example, different regions of a single gene, e.g., one domain targeting a promoter region and the other targeting a cis-regulatory region of the same gene), or different genes (e.g., one domain targeting a genetic element in one gene, and the other domain targeting a genetic element in a different gene).

In some embodiments, the fusion protein comprises a third DNA-binding zinc finger domain. In some embodiments, the first DNA-binding zinc finger domain, the second DNA-binding zinc finger domain, and the third DNA-binding zinc finger domain are the same. In some embodiments, the first DNA-binding zinc finger domain and the second DNA-binding zinc finger domain are the same. In some embodiments, the first DNA-binding zinc finger domain and the third DNA-binding zinc finger domain are the same. In some embodiments, the second DNA-binding zinc finger domain and the third DNA-binding zinc finger domain are the same. In some embodiments, each of the DNA-binding zinc finger domains is a different DNA-binding zinc finger domain. In some embodiments, the first, second, and third DNA-binding zinc finger domains target multiple sites in the same genetic element. In some embodiments, the first, second, and third DNA-binding zinc finger domains collectively target two or more genetic elements.

### II. Effector Domains

Fusion proteins described herein include one or more effector protein domains (also referred to herein as “effector domains”) that effect modification or modulation of a target gene. In some embodiments, the effector domain alters the genomic sequence, e.g., directly, for example via deamination of a nucleobase, or via cutting or nicking of the DNA and subsequent DNA repair resulting in a modification of the original DNA sequence. In some embodiments, the effector domain does not alter the DNA of a target gene or genetic sequence, but effects an epigenetic modification, such as a change in DNA methylation, histone methylation or acetylation, or chromatin state. Effector domains that do not alter the sequence of a substrate DNA target, but effect epigenetic changes, are referred to herein as “epigenetic effector domains,” and fusion proteins provided herein that comprise one or more epigenetic effector domains are referred to herein as “epigenetic editors.”

An epigenetic editor with one or more effector domains may modulate expression of a target gene without altering its nucleobase sequence. In some embodiments, an effector domain described herein may provide repression or silencing of expression of a target gene, e.g., by repressing transcription or by modifying or remodeling chromatin. Such effector domains are also referred to herein as “repression domains,” “repressor domains,” or “epigenetic repressor domains.” Non-limiting examples of chemical modifications that may be mediated by effector domains include methylation, demethylation, acetylation, deacetylation, phosphorylation, SUMOylation and/or ubiquitination of DNA or histone residues.

In some embodiments, an effector domain of an epigenetic editor described herein may make histone tail modifications, e.g., by adding or removing active marks on histone tails.

In some embodiments, an effector domain of an epigenetic editor described herein may comprise or recruit a transcription-related protein, e.g., a transcription repressor. The transcription-related protein may be endogenous or exogenous.

In some embodiments, an effector domain of an epigenetic editor described herein may, for example, comprise a protein that directly or indirectly blocks access of a transcription factor to the gene of interest harboring the target sequence.

An effector domain may be a full-length protein or a fragment thereof that retains the epigenetic effector function (a “functional domain”). Functional domains that are capable of modulating (e.g., repressing) gene expression can be derived from a larger protein. For example, functional domains that can reduce target gene expression may be identified based on sequences of repressor proteins. Amino acid sequences of gene expression-modulating proteins may be obtained from available genome browsers, such as the UCSD genome browser or Ensembl genome browser. Protein annotation databases such as UniProt or Pfam can be used to identify functional domains within the full protein sequence. As a starting point, the largest sequence, encompassing all regions identified by different databases, may be tested for gene expression modulation activity. Various truncations then may be tested to identify the minimal functional unit.

Variants of effector domains described herein are also contemplated by the present disclosure. A variant may, for example, refer to a polypeptide with at least about 85%, 90%, 91%, 92%, 93%, 94%, 95%, 96%, 97%, 98%, 99%, or 100% sequence identity and/or sequence similarity to a wildtype effector domain described herein. In particular embodiments, the variant retains at least about 85%, 90%, 91%, 92%, 93%, 94%, 95%, 96%, 97%, 98%, 99%, or 100% of the epigenetic effector function of the wildtype effector domain.

In some embodiments, an effector domain described herein may comprise a fusion of two or more effector domains (e.g., KOX1 KRAB and ZIM3). The effector domain may, for example, comprise a fusion of 2, 3, 4, 5, 6, 7, 8, 9, or 10 effector domains, such as effector domains described herein. In certain embodiments, an effector domain comprises a fusion of a truncated form of an effector domain and a second effector domain. In certain embodiments, an effector domain comprises a fusion of the truncated forms of two effector domains (e.g., fusions of the N- and C-terminal portions of the two effector domains).

In some embodiments, an epigenetic editor described herein may comprise 1 effector domain, 2 effector domains, 3 effector domains, 4 effector domains, 5 effector domains, 6 effector domains, 7 effector domains, 8 effector domains, 9 effector domains, 10 effector domains, or more. In certain embodiments, the epigenetic editor comprises one or more fusion proteins (e.g., one, two, or three fusion proteins), each with one or more effector domains (e.g., one, two, or three effector domains) linked to a DNA-binding domain. In some embodiments, the effector domains may induce a combination of epigenetic modifications, e.g., transcription repression and DNA methylation, DNA methylation and histone deacetylation, DNA methylation and histone demethylation, DNA methylation and histone methylation, DNA methylation and histone phosphorylation, DNA methylation and histone ubiquitylation, DNA methylation, and histone SUMOylation.

In certain embodiments, an effector domain described herein (e.g., DNMT3A and/or DNMT3L) is encoded by a nucleotide sequence as found in the native genome (e.g., human or murine) for that effector domain. In other embodiments, an effector domain described herein is encoded by a nucleotide sequence that has been codon-optimized for optimal expression in human cells.

Effector domains described herein may include, for example, transcriptional repressors, DNA methyltransferases, and/or histone modifiers, as further detailed below.

### A. Transcriptional Repressors

In some embodiments, an epigenetic effector domain described herein mediates repression of a target gene's expression (e.g., transcription). The effector domain may comprise, e.g., a Kruppel-associated box (KRAB) repressor domain, a Repressor Element Silencing Transcription Factor (REST) repressor domain, a KRAB-associated protein 1 (KAP1) domain, a MAD domain, a FKHR (forkhead in rhabdosarcoma gene) repressor domain, an EGR-1 (early growth response gene product-1) repressor domain, an ets2 repressor factor repressor domain (ERD), a MAD smSIN3 interaction domain (SID), a WRPW motif of the hairy-related basic helix-loop-helix (bHLH) repressor proteins, an HP1 alpha chromo-shadow repressor domain, an HP1 beta repressor domain, or any combination thereof. The effector domain may recruit one or more protein domains that repress expression of the target gene, e.g., through a scaffold protein. In some embodiments, the effector domain may recruit or interact with a scaffold protein domain that recruits a PRMT protein, a HDAC protein, a SETDB1 protein, or a NuRD protein domain.

In some embodiments, the effector domain comprises a functional domain derived from a zinc finger repressor protein, such as a KRAB domain. KRAB domains are found in approximately 400 human ZFP-based transcription factors. Descriptions of KRAB domains may be found, for example, in Ecco et al., Development (2017) 144(15):2719-29 and Lambert et al., Cell (2018) 172:650-65.

In certain embodiments, the effector domain comprises a repressor domain (e.g., KRAB) derived from KOX1/ZNF10, KOX8/ZNF708, ZNF43, ZNF184, ZNF91, HPF4, HTF10, or HTF34. In some embodiments, the effector domain comprises a repressor domain (e.g., KRAB) derived from ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37, ZNF34, ZNF250, ZNF547, ZNF273, ZNF354, ZFP82, ZNF224, ZNF33, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, or any combination thereof. For example, the repressor domain may be a KRAB domain derived from KOX1, ZIM3, ZFP28, or ZN627. In particular embodiments, the repressor domain is a ZIM3 KRAB domain. In further embodiments, the effector domain is derived from a human protein, e.g., a human ZIM3, a human KOX1, a human ZFP28, or a human ZN627.

Sequences of exemplary effector domains that may reduce or silence target gene expression, or protein sequences that contain them, are provided in Table 1 below (SEQ: SEQ ID NO). Further examples of repressors and transcriptional repressor domains can be found, e.g., in PCT Patent Publication WO 2021/226077 and Tycko et al., Cell (2020) 183(7):2020-35, each of which is incorporated herein by reference in its entirety.

TABLE 1
Exemplary Effector Domains That May
Reduce or Silence Gene Expression

| Protein | SEQ |
|---|---|
| ZIM3 | 33 |
| ZNF436 | 34 |
| ZNF257 | 35 |
| ZNF675 | 36 |
| ZNF490 | 37 |
| ZNF320 | 38 |
| ZNF331 | 39 |
| ZNF816 | 40 |
| ZNF680 | 41 |
| ZNF41 | 42 |
| ZNF189 | 43 |
| ZNF528 | 44 |
| ZNF543 | 45 |
| ZNF554 | 46 |
| ZNF140 | 47 |
| ZNF610 | 48 |
| ZNF264 | 49 |
| ZNF350 | 50 |
| ZNF8 | 51 |
| ZNF582 | 52 |
| ZNF30 | 53 |
| ZNF324 | 54 |
| ZNF98 | 55 |
| ZNF669 | 56 |
| ZNF677 | 57 |
| ZNF596 | 58 |
| ZNF214 | 59 |
| ZNF37A | 60 |
| ZNF34 | 61 |
| ZNF250 | 62 |
| ZNF547 | 63 |
| ZNF273 | 64 |
| ZNF354A | 65 |
| ZFP82 | 66 |
| ZNF224 | 67 |
| ZNF33A | 68 |
| ZNF45 | 69 |
| ZNF175 | 70 |
| ZNF595 | 71 |
| ZNF184 | 72 |
| ZNF419 | 73 |
| ZFP28-1 | 74 |
| ZFP28-2 | 75 |
| ZNF18 | 76 |
| ZNF213 | 77 |
| ZNF394 | 78 |
| ZFP1 | 79 |
| ZFP14 | 80 |
| ZNF416 | 81 |
| ZNF557 | 82 |
| ZNF566 | 83 |
| ZNF729 | 84 |
| ZIM2 | 85 |
| ZNF254 | 86 |
| ZNF764 | 87 |
| ZNF785 | 88 |
| ZNF10 (KOX1) | 89 |
| CBX5 (chromoshadow domain) | 90 |
| RYBP (YAF2_RYBP | 91 |
| component of PRC1) | |
| YAF2 (YAF2_RYBP | 92 |
| component of PRC1) | |
| MGA (component of PRC1.6) | 93 |
| CBX1 (chromoshadow) | 94 |
| SCMH1 (SAM_1/SPM) | 95 |
| MPP8 (Chromodomain) | 96 |
| SUMO3 (Rad60-SLD) | 97 |
| HERC2 (Cyt-b5) | 98 |
| BIN1 (SH3_9) | 99 |
| PCGF2 (RING finger protein | 100 |
| domain) | |
| TOX (HMG box) | 101 |
| FOXA1 (HNF3A C-terminal | 102 |
| domain) | |
| FOXA2 (HNF3B C-terminal | 103 |
| domain) | |
| IRF2BP1 (IRF-2BP1_2 N- | 104 |
| terminal domain) | |
| IRF2BP2 (IRF-2BP1_2 N- | 105 |
| terminal domain) | |
| IRF2BPL IRF-2BP1_2 N- | 106 |
| terminal domain | |
| HOXA13 (homeodomain) | 107 |
| HOXB13 (homeodomain) | 108 |
| HOXC13 (homeodomain) | 109 |
| HOXA11 (homeodomain) | 110 |
| HOXC11 (homeodomain) | 111 |
| HOXC10 (homeodomain) | 112 |
| HOXA10 (homeodomain) | 113 |
| HOXB9 (homeodomain) | 114 |
| HOXA9 (homeodomain) | 115 |
| ZFP28_HUMAN | 116 |
| ZN334_HUMAN | 117 |
| ZN568_HUMAN | 118 |
| ZN37A_HUMAN | 119 |
| ZN181_HUMAN | 120 |
| ZN510_HUMAN | 121 |
| ZN862_HUMAN | 122 |
| ZN140_HUMAN | 123 |
| ZN208_HUMAN | 124 |
| ZN248_HUMAN | 125 |
| ZN571_HUMAN | 126 |
| ZN699_HUMAN | 127 |
| ZN726_HUMAN | 128 |
| ZIK1_HUMAN | 129 |
| ZNF2_HUMAN | 130 |
| Z705F_HUMAN | 131 |
| ZNF14_HUMAN | 132 |
| ZN471_HUMAN | 133 |
| ZN624_HUMAN | 134 |
| ZNF84_HUMAN | 135 |
| ZNF7_HUMAN | 136 |
| ZN891_HUMAN | 137 |
| ZN337_HUMAN | 138 |
| Z705G_HUMAN | 139 |
| ZN529_HUMAN | 140 |
| ZN729_HUMAN | 141 |
| ZN419_HUMAN | 142 |
| Z705A_HUMAN | 143 |
| ZNF45_HUMAN | 144 |
| ZN302_HUMAN | 145 |
| ZN486_HUMAN | 146 |
| ZN621_HUMAN | 147 |
| ZN688_HUMAN | 148 |
| ZN33A_HUMAN | 149 |
| ZN554_HUMAN | 150 |
| ZN878_HUMAN | 151 |
| ZN772_HUMAN | 152 |
| ZN224_HUMAN | 153 |
| ZN184_HUMAN | 154 |
| ZN544_HUMAN | 155 |
| ZNF57_HUMAN | 156 |
| ZN283_HUMAN | 157 |
| ZN549_HUMAN | 158 |
| ZN211_HUMAN | 159 |
| ZN615_HUMAN | 160 |
| ZN253_HUMAN | 161 |
| ZN226_HUMAN | 162 |
| ZN730_HUMAN | 163 |
| Z585A_HUMAN | 164 |
| ZN732_HUMAN | 165 |
| ZN681_HUMAN | 166 |
| ZN667_HUMAN | 167 |
| ZN649_HUMAN | 168 |
| ZN470_HUMAN | 169 |
| ZN484_HUMAN | 170 |
| ZN431_HUMAN | 171 |
| ZN382_HUMAN | 172 |
| ZN254_HUMAN | 173 |
| ZN124_HUMAN | 174 |
| ZN607_HUMAN | 175 |
| ZN317_HUMAN | 176 |
| ZN620_HUMAN | 177 |
| ZN141_HUMAN | 178 |
| ZN584_HUMAN | 179 |
| ZN540_HUMAN | 180 |
| ZN75D_HUMAN | 181 |
| ZN555_HUMAN | 182 |
| ZN658_HUMAN | 183 |
| ZN684_HUMAN | 184 |
| RBAK_HUMAN | 185 |
| ZN829_HUMAN | 186 |
| ZN582_HUMAN | 187 |
| ZN112_HUMAN | 188 |
| ZN716_HUMAN | 189 |
| HKR1_HUMAN | 190 |
| ZN350_HUMAN | 191 |
| ZN480_HUMAN | 192 |
| ZN416_HUMAN | 193 |
| ZNF92_HUMAN | 194 |
| ZN100_HUMAN | 195 |
| ZN736_HUMAN | 196 |
| ZNF74_HUMAN | 197 |
| CBX1_HUMAN | 198 |
| ZN443_HUMAN | 199 |
| ZN195_HUMAN | 200 |
| ZN530_HUMAN | 201 |
| ZN782_HUMAN | 202 |
| ZN791_HUMAN | 203 |
| ZN331_HUMAN | 204 |
| Z354C_HUMAN | 205 |
| ZN157_HUMAN | 206 |
| ZN727_HUMAN | 207 |
| ZN550_HUMAN | 208 |
| ZN793_HUMAN | 209 |
| ZN235_HUMAN | 210 |
| ZNF8_HUMAN | 211 |
| ZN724_HUMAN | 212 |
| ZN573_HUMAN | 213 |
| ZN577_HUMAN | 214 |
| ZN789_HUMAN | 215 |
| ZN718_HUMAN | 216 |
| ZN300_HUMAN | 217 |
| ZN383_HUMAN | 218 |
| ZN429_HUMAN | 219 |
| ZN677_HUMAN | 220 |
| ZN850_HUMAN | 221 |
| ZN454_HUMAN | 222 |
| ZN257_HUMAN | 223 |
| ZN264_HUMAN | 224 |
| ZFP82_HUMAN | 225 |
| ZFP14_HUMAN | 226 |
| ZN485_HUMAN | 227 |
| ZN737_HUMAN | 228 |
| ZNF44_HUMAN | 229 |
| ZN596_HUMAN | 230 |
| ZN565_HUMAN | 231 |
| ZN543_HUMAN | 232 |
| ZFP69_HUMAN | 233 |
| SUMO1_HUMAN | 234 |
| ZNF12_HUMAN | 235 |
| ZN169_HUMAN | 236 |
| ZN433_HUMAN | 237 |
| SUMO3_HUMAN | 238 |
| ZNF98_HUMAN | 239 |
| ZN175_HUMAN | 240 |
| ZN347_HUMAN | 241 |
| ZNF25_HUMAN | 242 |
| ZN519_HUMAN | 243 |
| Z585B_HUMAN | 244 |
| ZIM3_HUMAN | 245 |
| ZN517_HUMAN | 246 |
| ZN846_HUMAN | 247 |
| ZN230_HUMAN | 248 |
| ZNF66_HUMAN | 249 |
| ZFP1_HUMAN | 250 |
| ZN713_HUMAN | 251 |
| ZN816_HUMAN | 252 |
| ZN426_HUMAN | 253 |
| ZN674_HUMAN | 254 |
| ZN627_HUMAN | 255 |
| ZNF20_HUMAN | 256 |
| Z587B_HUMAN | 257 |
| ZN316_HUMAN | 258 |
| ZN233_HUMAN | 259 |
| ZN611_HUMAN | 260 |
| ZN556_HUMAN | 261 |
| ZN234_HUMAN | 262 |
| ZN560_HUMAN | 263 |
| ZNF77_HUMAN | 264 |
| ZN682_HUMAN | 265 |
| ZN614_HUMAN | 266 |
| ZN785_HUMAN | 267 |
| ZN445_HUMAN | 268 |
| ZFP30_HUMAN | 269 |
| ZN225_HUMAN | 270 |
| ZN551_HUMAN | 271 |
| ZN610_HUMAN | 272 |
| ZN528_HUMAN | 273 |
| ZN284_HUMAN | 274 |
| ZN418_HUMAN | 275 |
| MPP8_HUMAN | 276 |
| ZN490_HUMAN | 277 |
| ZN805_HUMAN | 278 |
| Z780B_HUMAN | 279 |
| ZN763_HUMAN | 280 |
| ZN285_HUMAN | 281 |
| ZNF85_HUMAN | 282 |
| ZN223_HUMAN | 283 |
| ZNF90_HUMAN | 284 |
| ZN557_HUMAN | 285 |
| ZN425_HUMAN | 286 |
| ZN229_HUMAN | 287 |
| ZN606_HUMAN | 288 |
| ZN155_HUMAN | 289 |
| ZN222_HUMAN | 290 |
| ZN442_HUMAN | 291 |
| ZNF91_HUMAN | 292 |
| ZN135_HUMAN | 293 |
| ZN778_HUMAN | 294 |
| RYBP_HUMAN | 295 |
| ZN534_HUMAN | 296 |
| ZN586_HUMAN | 297 |
| ZN567_HUMAN | 298 |
| ZN440_HUMAN | 299 |
| ZN583_HUMAN | 300 |
| ZN441_HUMAN | 301 |
| ZNF43_HUMAN | 302 |
| CBX5_HUMAN | 303 |
| ZN589_HUMAN | 304 |
| ZNF10_HUMAN | 305 |
| ZN563_HUMAN | 306 |
| ZN561_HUMAN | 307 |
| ZN136_HUMAN | 308 |
| ZN630_HUMAN | 309 |
| ZN527_HUMAN | 310 |
| ZN333_HUMAN | 311 |
| Z324B_HUMAN | 312 |
| ZN786_HUMAN | 313 |
| ZN709_HUMAN | 314 |
| ZN792_HUMAN | 315 |
| ZN599_HUMAN | 316 |
| ZN613_HUMAN | 317 |
| ZF69B_HUMAN | 318 |
| ZN799_HUMAN | 319 |
| ZN569_HUMAN | 320 |
| ZN564_HUMAN | 321 |
| ZN546_HUMAN | 322 |
| ZFP92_HUMAN | 323 |
| YAF2_HUMAN | 324 |
| ZN723_HUMAN | 325 |
| ZNF34_HUMAN | 326 |
| ZN439_HUMAN | 327 |
| ZFP57_HUMAN | 328 |
| ZNF19_HUMAN | 329 |
| ZN404_HUMAN | 330 |
| ZN274_HUMAN | 331 |
| CBX3_HUMAN | 332 |
| ZNF30_HUMAN | 333 |
| ZN250_HUMAN | 334 |
| ZN570_HUMAN | 335 |
| ZN675_HUMAN | 336 |
| ZN695_HUMAN | 337 |
| ZN548_HUMAN | 338 |
| ZN132_HUMAN | 339 |
| ZN738_HUMAN | 340 |
| ZN420_HUMAN | 341 |
| ZN626_HUMAN | 342 |
| ZN559_HUMAN | 343 |
| ZN460_HUMAN | 344 |
| ZN268_HUMAN | 345 |
| ZN304_HUMAN | 346 |
| ZIM2_HUMAN | 347 |
| ZN605_HUMAN | 348 |
| ZN844_HUMAN | 349 |
| SUMO5_HUMAN | 350 |
| ZN101_HUMAN | 351 |
| ZN783_HUMAN | 352 |
| ZN417_HUMAN | 353 |
| ZN182_HUMAN | 354 |
| ZN823_HUMAN | 355 |
| ZN177_HUMAN | 356 |
| ZN197_HUMAN | 357 |
| ZN717_HUMAN | 358 |
| ZN669_HUMAN | 359 |
| ZN256_HUMAN | 360 |
| ZN251_HUMAN | 361 |
| CBX4_HUMAN | 362 |
| PCGF2_HUMAN | 363 |
| CDY2_HUMAN | 364 |
| CDYL2_HUMAN | 365 |
| HERC2_HUMAN | 366 |
| ZN562_HUMAN | 367 |
| ZN461_HUMAN | 368 |
| Z324A_HUMAN | 369 |
| ZN766_HUMAN | 370 |
| ID2_HUMAN | 371 |
| TOX_HUMAN | 372 |
| ZN274_HUMAN | 373 |
| SCMH1_HUMAN | 374 |
| ZN214_HUMAN | 375 |
| CBX7_HUMAN | 376 |
| ID1_HUMAN | 377 |
| CREM_HUMAN | 378 |
| SCX_HUMAN | 379 |
| ASCL1_HUMAN | 380 |
| ZN764_HUMAN | 381 |
| SCML2_HUMAN | 382 |
| TWST1_HUMAN | 383 |
| CREB1_HUMAN | 384 |
| TERF1_HUMAN | 385 |
| ID3_HUMAN | 386 |
| CBX8_HUMAN | 387 |
| CBX4_HUMAN | 388 |
| GSX1_HUMAN | 389 |
| NKX22_HUMAN | 390 |
| ATF1_HUMAN | 391 |
| TWST2_HUMAN | 392 |
| ZNF17_HUMAN | 393 |
| TOX3_HUMAN | 394 |
| TOX4_HUMAN | 395 |
| ZMYM3_HUMAN | 396 |
| I2BP1_HUMAN | 397 |
| RHXF1_HUMAN | 398 |
| SSX2_HUMAN | 399 |
| I2BPL_HUMAN | 400 |
| ZN680_HUMAN | 401 |
| CBX1_HUMAN | 402 |
| TRI68_HUMAN | 403 |
| HXA13_HUMAN | 404 |
| PHC3_HUMAN | 405 |
| TCF24_HUMAN | 406 |
| CBX3_HUMAN | 407 |
| HXB13_HUMAN | 408 |
| HEY1_HUMAN | 409 |
| PHC2_HUMAN | 410 |
| ZNF81_HUMAN | 411 |
| FIGLA_HUMAN | 412 |
| SAM11_HUMAN | 413 |
| KMT2B_HUMAN | 414 |
| HEY2_HUMAN | 415 |
| JDP2_HUMAN | 416 |
| HXC13_HUMAN | 417 |
| ASCL4_HUMAN | 418 |
| HHEX_HUMAN | 419 |
| HERC2_HUMAN | 420 |
| GSX2_HUMAN | 421 |
| BIN1_HUMAN | 422 |
| ETV7_HUMAN | 423 |
| ASCL3_HUMAN | 424 |
| PHC1_HUMAN | 425 |
| OTP_HUMAN | 426 |
| I2BP2_HUMAN | 427 |
| VGLL2_HUMAN | 428 |
| HXA11_HUMAN | 429 |
| PDLI4_HUMAN | 430 |
| ASCL2_HUMAN | 431 |
| CDX4_HUMAN | 432 |
| ZN860_HUMAN | 433 |
| LMBL4_HUMAN | 434 |
| PDIP3_HUMAN | 435 |
| NKX25_HUMAN | 436 |
| CEBPB_HUMAN | 437 |
| ISL1_HUMAN | 438 |
| CDX2_HUMAN | 439 |
| PROP1_HUMAN | 440 |
| SIN3B_HUMAN | 441 |
| SMBT1_HUMAN | 442 |
| HXC11_HUMAN | 443 |
| HXC10_HUMAN | 444 |
| PRS6A_HUMAN | 445 |
| VSX1_HUMAN | 446 |
| NKX23_HUMAN | 447 |
| MTG16_HUMAN | 448 |
| HMX3_HUMAN | 449 |
| HMX1_HUMAN | 450 |
| KIF22_HUMAN | 451 |
| CSTF2_HUMAN | 452 |
| CEBPE_HUMAN | 453 |
| DLX2_HUMAN | 454 |
| ZMYM3_HUMAN | 455 |
| PPARG_HUMAN | 456 |
| PRIC1_HUMAN | 457 |
| UNC4_HUMAN | 458 |
| BARX2_HUMAN | 459 |
| ALX3_HUMAN | 460 |
| TCF15_HUMAN | 461 |
| TERA_HUMAN | 462 |
| VSX2_HUMAN | 463 |
| HXD12_HUMAN | 464 |
| CDX1_HUMAN | 465 |
| TCF23_HUMAN | 466 |
| ALX1_HUMAN | 467 |
| HXA10_HUMAN | 468 |
| RX_HUMAN | 469 |
| CXXC5_HUMAN | 470 |
| SCML1_HUMAN | 471 |
| NFIL3_HUMAN | 472 |
| DLX6_HUMAN | 473 |
| MTG8_HUMAN | 474 |
| CBX8_HUMAN | 475 |
| CEBPD_HUMAN | 476 |
| SEC13_HUMAN | 477 |
| FIP1_HUMAN | 478 |
| ALX4_HUMAN | 479 |
| LHX3_HUMAN | 480 |
| PRIC2_HUMAN | 481 |
| MAGI3_HUMAN | 482 |
| NELL1_HUMAN | 483 |
| PRRX1_HUMAN | 484 |
| MTG8R_HUMAN | 485 |
| RAX2_HUMAN | 486 |
| DLX3_HUMAN | 487 |
| DLX1_HUMAN | 488 |
| NKX26_HUMAN | 489 |
| NAB1_HUMAN | 490 |
| SAMD7_HUMAN | 491 |
| PITX3_HUMAN | 492 |
| WDR5_HUMAN | 493 |
| MEOX2_HUMAN | 494 |
| NAB2_HUMAN | 495 |
| DHX8_HUMAN | 496 |
| FOXA2_HUMAN | 497 |
| CBX6_HUMAN | 498 |
| EMX2_HUMAN | 499 |
| CPSF6_HUMAN | 500 |
| HXC12_HUMAN | 501 |
| KDM4B_HUMAN | 502 |
| LMBL3_HUMAN | 503 |
| PHX2A_HUMAN | 504 |
| EMX1_HUMAN | 505 |
| NC2B_HUMAN | 506 |
| DLX4_HUMAN | 507 |
| SRY_HUMAN | 508 |
| ZN777_HUMAN | 509 |
| NELL1_HUMAN | 510 |
| ZN398_HUMAN | 511 |
| GATA3_HUMAN | 512 |
| BSH_HUMAN | 513 |
| SF3B4_HUMAN | 514 |
| TEAD1_HUMAN | 515 |
| TEAD3_HUMAN | 516 |
| RGAP1_HUMAN | 517 |
| PHF1_HUMAN | 518 |
| FOXA1_HUMAN | 519 |
| GATA2_HUMAN | 520 |
| FOXO3_HUMAN | 521 |
| ZN212_HUMAN | 522 |
| IRX4_HUMAN | 523 |
| ZBED6_HUMAN | 524 |
| LHX4_HUMAN | 525 |
| SIN3A_HUMAN | 526 |
| RBBP7_HUMAN | 527 |
| NKX61_HUMAN | 528 |
| TRI68_HUMAN | 529 |
| R51A1_HUMAN | 530 |
| MB3L1_HUMAN | 531 |
| DLX5_HUMAN | 532 |
| NOTC1_HUMAN | 533 |
| TERF2_HUMAN | 534 |
| ZN282_HUMAN | 535 |
| RGS12_HUMAN | 536 |
| ZN840_HUMAN | 537 |
| SPI2B_HUMAN_1 | 538 |
| PAX7_HUMAN | 539 |
| NKX62_HUMAN | 540 |
| ASXL2_HUMAN | 541 |
| FOXO1_HUMAN | 542 |
| GATA3_HUMAN | 543 |
| GATA1_HUMAN | 544 |
| ZMYM5_HUMAN | 545 |
| ZN783_HUMAN | 546 |
| SPI2B_HUMAN_2 | 547 |
| LRP1_HUMAN | 548 |
| MIXL1_HUMAN | 549 |
| SGT1_HUMAN | 550 |
| LMCD1_HUMAN | 551 |
| CEBPA_HUMAN | 552 |
| GATA2_HUMAN | 553 |
| SOX14_HUMAN | 554 |
| WTIP_HUMAN | 555 |
| PRP19_HUMAN | 556 |
| CBX6_HUMAN | 557 |
| NKX11_HUMAN | 558 |
| RBBP4_HUMAN | 559 |
| DMRT2_HUMAN | 560 |
| SMCA2_HUMAN | 561 |
| ZNF10_HUMAN | 562 |
| EED_HUMAN | 563 |
| RCOR1_HUMAN | 564 |

A functional analog of any one of the above-listed proteins, i.e., a molecule having the same or substantially the same biological function (e.g., retaining 70% or more, 80% or more, 90% or more, 95% or more, or 98% or more) of the protein's transcription factor function) is encompassed by the present disclosure. For example, the functional analog may be an isoform or a variant of the above-listed protein, e.g., containing a portion of the above protein with or without additional amino acid residues and/or containing mutations relative to the above protein. In some embodiments, the functional analog has a sequence identity that is at least 75, 80, 85, 90, 95, 98, or 99% to one of the sequences listed in Table 1. Homologs, orthologs, and mutants of the above-listed proteins are also contemplated.

In certain embodiments, an epigenetic editor described herein comprises a KRAB domain derived from KOX1, ZIM3, ZFP28, or ZN627, and/or an effector domain derived from KAP1, MECP2, HP1a, HP1b, CBX8, CDYL2, TOX, TOX3, TOX4, EED, EZH2, RBBP4, RCOR1, or SCML2, optionally wherein the parental protein is a human protein. In particular embodiments, an epigenetic editor described herein comprises a domain derived from KOX1, ZIM3, ZFP28, and/or ZN627, optionally wherein the parental protein is a human protein. In certain embodiments, the epigenetic editor may comprise a KRAB domain derived from KOX1 (ZNF10), e.g., a human KOX1. In certain embodiments, the epigenetic editor may comprise a KRAB domain derived from ZIM3 (ZNF657 or ZNF264), e.g., a human ZIM3. In certain embodiments, the epigenetic editor may comprise a KRAB domain derived from ZFP28, e.g., a human ZFP28. In certain embodiments, the epigenetic editor may comprise a KRAB domain derived from ZN627, e.g., a human ZN627. In certain embodiments, an epigenetic editor described herein may comprise a CDYL2, e.g., a human CDYL2, and/or a TOX domain (e.g., a human TOX domain) in combination with a KOX1 KRAB domain (e.g., a human KOX1 KRAB domain).

In certain embodiments, an epigenetic effector described herein comprises a repressor domain derived from KOX1/ZNF10 (SEQ ID NO: 89). For example, the repressor domain may comprise the sequence of SEQ ID NO: 89, or a sequence at least 75%, 80%, 85%, 90%, 91%, 92%, 93%, 94%, 95%, 96%, 97%, 98%, or 99% identical to SEQ ID NO: 89.

In certain embodiments, an epigenetic effector described herein comprises a repressor domain derived from KOX1/ZNF10, as shown in Table 2 below:

TABLE 2
Exemplary Effector Domains Derived from KOX1/ZNF10

| Protein | Protein Sequence |
|---|---|
| KOX1/ZNF10 KRAB 1 | SEQ ID NO: 565 |
| KOX1/ZNF10 KRAB 2 | SEQ ID NO: 566 |
| KOX1/ZNF10 KRAB 3 | SEQ ID NO: 567 |
| KOX1/ZNF10 (aa 11-72) | SEQ ID NO: 568 |
| KOX1/ZNF10 (aa 11-108) | SEQ ID NO: 569 |
| KOX1/ZNF10 variant | SEQ ID NO: 570 |
| KOX1 KRAB-ZIM3 chimera | SEQ ID NO: 571 |
| ZIM3-KOX1 KRAB chimera | SEQ ID NO: 572 |

In particular embodiments, the repressor domain may comprise the amino acid sequence of SEQ ID NO: 565, or a sequence at least 75%, 80%, 85%, 90%, 91%, 92%, 93%, 94%, 95%, 96%, 97%, 98%, or 99% identical to SEQ ID NO: 565.

In particular embodiments, the repressor domain may comprise the amino acid sequence of SEQ ID NO: 566, or a sequence at least 75%, 80%, 85%, 90%, 91%, 92%, 93%, 94%, 95%, 96%, 97%, 98%, or 99% identical to SEQ ID NO: 566.

In particular embodiments, the repressor domain may comprise the amino acid sequence of SEQ ID NO: 567, or a sequence at least 75%, 80%, 85%, 90%, 91%, 92%, 93%, 94%, 95%, 96%, 97%, 98%, or 99% identical to SEQ ID NO: 567.

In particular embodiments, the repressor domain may comprise the amino acid sequence of SEQ ID NO: 568, or a sequence at least 75%, 80%, 85%, 90%, 91%, 92%, 93%, 94%, 95%, 96%, 97%, 98%, or 99% identical to SEQ ID NO: 568.

In particular embodiments, the repressor domain may comprise the amino acid sequence of SEQ ID NO: 569, or a sequence at least 75%, 80%, 85%, 90%, 91%, 92%, 93%, 94%, 95%, 96%, 97%, 98%, or 99% identical to SEQ ID NO: 569.

In particular embodiments, the repressor domain may comprise the amino acid sequence of SEQ ID NO: 570, or a sequence at least 75%, 80%, 85%, 90%, 91%, 92%, 93%, 94%, 95%, 96%, 97%, 98%, or 99% identical to SEQ ID NO: 570.

In particular embodiments, the repressor domain may comprise the amino acid sequence of SEQ ID NO: 571, or a sequence at least 75%, 80%, 85%, 90%, 91%, 92%, 93%, 94%, 95%, 96%, 97%, 98%, or 99% identical to SEQ ID NO: 571.

In particular embodiments, the repressor domain may comprise the amino acid sequence of SEQ ID NO: 572, or a sequence at least 75%, 80%, 85%, 90%, 91%, 92%, 93%, 94%, 95%, 96%, 97%, 98%, or 99% identical to SEQ ID NO: 572.

### B. DNA Methyltransferases

In some embodiments, an effector domain of an epigenetic editor described herein alters target gene expression through DNA modification, such as methylation. Highly methylated areas of DNA tend to be less transcriptionally active than less methylated areas. DNA methylation occurs primarily at CpG sites (shorthand for “C-phosphate-G-” or “cytosine-phosphate-guanine” sites). Many mammalian genes have promoter regions near or including CpG islands (nucleic acid regions with a high frequency of CpG dinucleotides).

An effector domain described herein may be, e.g., a DNA methyltransferase (DNMT) or a catalytic domain thereof, or may be capable of recruiting a DNA methyltransferase. DNMTs encompass enzymes that catalyze the transfer of a methyl group to a DNA nucleotide, such as canonical cytosine-5 DNMTs that catalyze the addition of methyl groups to genomic DNA (e.g., DNMT1, DNMT3A, DNMT3B, and DNMT3C). This term also encompasses non-canonical family members that do not catalyze methylation themselves but that recruit (including activate) catalytically active DNMTs; a non-limiting examples of such a DNMT is DNMT3L. See, e.g., Lyko, Nat Review (2018) 19:81-92. Unless otherwise indicated, a DNMT domain may refer to a polypeptide domain derived from a catalytically active DNMT (e.g., DNMT1, DNMT3A, and DNMT3B) or from a catalytically inactive DNMT (e.g., DNMT3L). A DNMT may repress expression of the target gene through the recruitment of repressive regulatory proteins. In some embodiments, the methylation is at a CG (or CpG) dinucleotide sequence. In some embodiments, the methylation is at a CHG or CHH sequence, where H is any one of A, T, or C.

In some embodiments, a DNMT described herein can be an animal DNMT (e.g., a mammalian DNMT), a plant DNMT, a fungal DNMT, or a bacterial DNMT. A bacterial DNMT can be obtained from a bacterial species (e.g., a coccus bacterium, bacillus bacterium, spiral bacterium, or an intracellular, gram-positive, or gram-negative bacterium. In certain embodiments, the bacterial species is Mycoplasmatales bacterium, Mycoplasma marinum, or Spiroplasma chinense. In certain embodiments, the bacterial species is not M. penetrans, S. monbiae, H. parainfluenzae, A. luteus, H. aegyptius, H. haemolyticus, Moraxella, E. coli, T. aquaticus, C. crescentus, or C. difficile. In certain embodiments, an epigenetic editor described herein comprises a DNMT domain comprising SEQ ID NO: 601, or a sequence at least 75%, 80%, 85%, 90%, 91%, 92%, 93%, 94%, 95%, 96%, 97%, 98%, or 99% identical to SEQ ID NO: 601. In certain embodiments, an epigenetic editor described herein comprises a DNMT domain comprising SEQ ID NO: 602, or a sequence at least 75%, 80%, 85%, 90%, 91%, 92%, 93%, 94%, 95%, 96%, 97%, 98%, or 99% identical to SEQ ID NO: 602. In certain embodiments, an epigenetic editor described herein comprises a DNMT domain comprising SEQ ID NO: 603, or a sequence at least 75%, 80%, 85%, 90%, 91%, 92%, 93%, 94%, 95%, 96%, 97%, 98%, or 99% identical to SEQ ID NO: 603.

In certain embodiments, DNMTs in the epigenetic editors described herein may include, e.g., DNMT1, DNMT3A, DNMT3B, and/or DNMT3C. In some embodiments, the DNMT is a mammalian (e.g., human or murine) DNMT. In particular embodiments, the DNMT is DNMT3A (e.g., human DNMT3A). In certain embodiments, an epigenetic editor described herein comprises a DNMT3A domain comprising SEQ ID NO: 574, or a sequence at least 75%, 80%, 85%, 90%, 91%, 92%, 93%, 94%, 95%, 96%, 97%, 98%, or 99% identical to SEQ ID NO: 574. In certain embodiments, an epigenetic editor described herein comprises a DNMT3A domain comprising SEQ ID NO: 575, or a sequence at least 75%, 80%, 85%, 90%, 91%, 92%, 93%, 94%, 95%, 96%, 97%, 98%, or 99% identical to SEQ ID NO: 575. In some embodiments, the DNMT3A domain may have, e.g., a mutation at position H739 (such as H739A or H739E), R771 (such as R771L) and/or R836 (such as R836A or R836Q), or any combination thereof (numbering according to SEQ ID NO: 574).

In some embodiments, an effector domain described herein may be a DNMT-like domain. As used herein a “DNMT-like domain” is a regulatory factor of DNMT that may activate or recruit other DNMT domains, but does not itself possess methylation activity. In some embodiments, the DNMT-like domain is a mammalian (e.g., human or mouse) DNMT-like domain. In certain embodiments, the DNMT-like domain is DNMT3L, which may be, for example, human DNMT3L or mouse DNMT3L. In certain embodiments, an epigenetic editor described herein comprises a DNMT3L domain comprising SEQ ID NO: 578, or a sequence at least 75%, 80%, 85%, 90%, 91%, 92%, 93%, 94%, 95%, 96%, 97%, 98%, or 99% identical to SEQ ID NO: 578. In certain embodiments, an epigenetic editor herein comprises a DNMT3L domain comprising SEQ ID NO: 579, or a sequence at least 75%, 80%, 85%, 90%, 91%, 92%, 93%, 94%, 95%, 96%, 97%, 98%, or 99% identical to SEQ ID NO: 579. In certain embodiments, an epigenetic editor described herein comprises a DNMT3L domain comprising SEQ ID NO: 580, or a sequence at least 75%, 80%, 85%, 90%, 91%, 92%, 93%, 94%, 95%, 96%, 97%, 98%, or 99% identical to SEQ ID NO: 580. In certain embodiments, an epigenetic editor described herein comprises a DNMT3L domain comprising SEQ ID NO: 581, or a sequence at least 75%, 80%, 85%, 90%, 91%, 92%, 93%, 94%, 95%, 96%, 97%, 98%, or 99% identical to SEQ ID NO: 581. In some embodiments, the DNMT3L domain may have, e.g., a mutation corresponding to that at position D226 (such as D226V), Q268 (such as Q268K), or both (numbering according to SEQ ID NO: 578).

In certain embodiments, an epigenetic editor herein may comprise comprising both DNMT and DNMT-like effector domains. For example, the epigenetic editor may comprise a DNMT3A-3L domain, wherein DNMT3A and DNMT3L may be covalently linked. In other embodiments, an epigenetic editor described herein may comprise an effector domain that comprises only a DNMT3A domain (e.g., human DNMT3A), or only a DNMT-like domain (e.g., DNMT3L, which may be human or mouse DNMT3L).

Table 3 below provides exemplary DNMTs that may be part of an epigenetic effector domain described herein, or from which an effector domain of an epigenetic editor described herein may be derived.

TABLE 3
Exemplary DNMT Sequences

| Protein Name | Species | Target | Protein Sequence |
|---|---|---|---|
| DNMT1 | Human | 5mC | SEQ ID NO: 573 |
| DNMT3A (h3A) | Human | 5mC | SEQ ID NO: 574 |
| DNMT3A | Human | 5mC | SEQ ID NO: 575 |
| (catalytic domain) | | | |
| (h3As) | | | |
| DNMT3B | Human | 5mC | SEQ ID NO: 576 |
| DNMT3C | Mouse | 5mC | SEQ ID NO: 577 |
| DNMT3L (h3L) | Human | 5mC | SEQ ID NO: 578 |
| DNMT3L | Human | 5mC | SEQ ID NO: 579 |
| (catalytic domain) | | | |
| (h3Ls) | | | |
| DNMT3L (m3L) | Mouse | 5mC | SEQ ID NO: 580 |
| DNMT3L | Mouse | 5mC | SEQ ID NO: 581 |
| (catalytic domain) | | | |
| (m3Ls) | | | |
| DNMT3L | Ailuropoda melanoleuca | 5mC | SEQ ID NO: 582 |
| DNMT3L | Ailuropoda melanoleuca | 5mC | SEQ ID NO: 583 |
| (catalytic domain) | | | |
| DNMT3L | Carlito syrichta | 5mC | SEQ ID NO: 584 |
| DNMT3L | Carlito syrichta | 5mC | SEQ ID NO: 585 |
| (catalytic domain) | | | |
| DNMT3L | Meriones unguiculatus | 5mC | SEQ ID NO: 586 |
| DNMT3L | Meriones unguiculatus | 5mC | SEQ ID NO: 587 |
| (catalytic domain) | | | |
| DNMT3L | Ochotona princeps | 5mC | SEQ ID NO: 588 |
| DNMT3L | Ochotona princeps | 5mC | SEQ ID NO: 589 |
| (catalytic domain) | | | |
| DNMT3L | Neosciurus carolinensis | 5mC | SEQ ID NO: 590 |
| DNMT3L | Neosciurus carolinensis | 5mC | SEQ ID NO: 591 |
| (catalytic domain) | | | |
| DNMT3L | Bison bison | 5mC | SEQ ID NO: 592 |
| DNMT3L | Bison bison | 5mC | SEQ ID NO: 593 |
| (catalytic domain) | | | |
| DNMT3L | Equus przewalskii | 5mC | SEQ ID NO: 594 |
| DNMT3L | Equus przewalskii | 5mC | SEQ ID NO: 595 |
| (catalytic domain) | | | |
| DNMT3L | Mus caroli | 5mC | SEQ ID NO: 596 |
| DNMT3L | Mus caroli | 5mC | SEQ ID NO: 597 |
| (catalytic domain) | | | |
| DNMT3L | Pan troglodytes | 5mC | SEQ ID NO: 598 |
| DNMT3L | Pan troglodytes | 5mC | SEQ ID NO: 599 |
| (catalytic domain) | | | |
| TRDMT1 | Human | tRNA 5mC | SEQ ID NO: 600 |
| (DNMT2) | | | |
| DNA cytosine | Mycoplasmatales | 5mC | SEQ ID NO: 601 |
| methyltransferase | bacterium | | |
| DNA cytosine | Mycoplasma marinum | 5mC | SEQ ID NO: 602 |
| methyltransferase | | | |
| DNA (cytosine-5-)- | Spiroplasma chinense | 5mC | SEQ ID NO: 603 |
| methyltransferase | | | |
| M.MpeI | Mycoplasma penetrans | 5mC | SEQ ID NO: 604 |
| M.SssI | Spiroplasma monobiae | 5mC | SEQ ID NO: 605 |
| M.HpaII | Haemophilus | 5mC (CCGG) | SEQ ID NO: 606 |
| | parainfluenzae | | |
| M.AluI | Arthrobacter luteus | 5mC (AGCT) | SEQ ID NO: 607 |
| M.HaeIII | Haemophilus aegyptius | 5mC (GGCC) | SEQ ID NO: 608 |
| M.HhaI | Haemophilus | 5mC (GCGC) | SEQ ID NO: 609 |
| | haemolyticus | | |
| M.MspI | Moraxella | 5mC (CCGG) | SEQ ID NO: 610 |
| Masc1 | Ascobolus | 5mC | SEQ ID NO: 611 |
| MET1 | Arabidopsis | 5mC | SEQ ID NO: 612 |
| Masc2 | Ascobolus | 5mC | SEQ ID NO: 613 |
| Dim-2 | Neurospora | 5mC | SEQ ID NO: 614 |
| dDnmt2 | Drosophila | 5mC | SEQ ID NO: 615 |
| Pmt1 | S. pombe | 5mC | SEQ ID NO: 616 |
| DRM1 | Arabidopsis | 5mC | SEQ ID NO: 617 |
| DRM2 | Arabidopsis | 5mC | SEQ ID NO: 618 |
| CMT1 | Arabidopsis | 5mC | SEQ ID NO: 619 |
| CMT2 | Arabidopsis | 5mC | SEQ ID NO: 620 |
| CMT3 | Arabidopsis | 5mC | SEQ ID NO: 621 |
| Rid | Neurospora | 5mC | SEQ ID NO: 622 |
| hsdM gene | bacteria (E. coli, strain 12) | m6A | SEQ ID NO: 623 |
| hsdS gene | bacteria (E. coli, strain 12) | m6A | SEQ ID NO: 624 |
| M.TaqI | Bacteria (Thermus | m6A | SEQ ID NO: 625 |
| | aquaticus) | | |
| M.EcoDam | E. coli | m6A | SEQ ID NO: 626 |
| M.CcrMI | Caulobacter crescentus | m6A | SEQ ID NO: 627 |
| CamA | Clostridioides difficile | m6A | SEQ ID NO: 628 |

A functional analog of any one of the above-listed proteins, i.e., a molecule having the same or substantially the same biological function (e.g., retaining 70% or more, 80% or more, 90% or more, 95% or more, or 98% or more) of the protein's DNA methylation function or recruiting function) is encompassed by the present disclosure. For example, the functional analog may be an isoform or a variant of the above-listed protein, e.g., containing a portion of the above protein with or without additional amino acid residues and/or containing mutations relative to the above protein. In some embodiments, the functional analog has a sequence identity that is at least 75, 80, 85, 90, 95, 98, or 99% to one of the sequences listed in Table 3. In some embodiments, the effector domain herein comprises only the functional domain (or functional analog thereof), e.g., the catalytic domain or recruiting domain, of an above-listed protein. In some embodiments, the effector domain herein comprises one or more epigenetic effector domains selected from Table 3, or functional homologs, orthologs, or variants thereof.

As used herein, a DNMT domain (e.g., a DNMT3A domain or a DNMT3L domain) refers to a protein domain that is identical to the parental protein (e.g., a human or murine DNMT3A or DNMT3L) or a functional analog thereof (e.g., having a functional fragment, such as a catalytic fragment or recruiting fragment, of the parental protein; and/or having mutations that improve the activity of the DNMT protein).

An epigenetic editor herein may effect methylation at, e.g., 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 or more CpG dinucleotide sequences in the target gene or chromosome. The CpG dinucleotide sequences may be located within or near the target gene in CpG islands, or may be located in a region that is not a CpG island. A CpG island generally refers to a nucleic acid sequence or chromosome region that comprises a high frequency of CpG dinucleotides. For example, a CpG island may comprise at least 50% GC content. The CpG island may have a high observed-to-expected CpG ratio, for example, an observed-to-expected CpG ratio of at least 60%. As used herein, an observed-to-expected CpG ratio is determined by Number of CpG*(sequence length)/(Number of C*Number of G). In some embodiments, the CpG island has an observed-to-expected CpG ratio of at least 60%, 70%, 80%, 90% or more. A CpG island may be a sequence or region of, e.g., at least 200, 250, 300, 350, 400, 450, 500, 550, 600, 650, 700, 750, or 800 nucleotides. In some embodiments, only 1, or less than 2, 3, 4, 5, 6, 7, 8, 9, 10, 11, 12, 13, 14, 15, 16, 17, 18, 19, 20, 30, 40, or 50 CpG dinucleotides are methylated by the epigenetic editor.

In some embodiments, an epigenetic editor herein effects methylation at a hypomethylated nucleic acid sequence, i.e., a sequence that may lack methyl groups on the 5-methyl cytosine nucleotides (e.g., in CpG) as compared to a standard control. Hypomethylation may occur, for example, in aging cells or in cancer (e.g., early stages of neoplasia) relative to a younger cell or non-cancer cell, respectively.

In some embodiments, an epigenetic editor described herein induces methylation at a hypermethylated nucleic acid sequence.

In some embodiments, methylation may be introduced by the epigenetic editor at a site other than a CpG dinucleotide. For example, the target gene sequence may be methylated at the C nucleotide of CpA, CpT, or CpC sequences. In some embodiments, an epigenetic editor comprises a DNMT3A domain and effects methylation at CpG, CpA, CpT, CpC sequences, or any combination thereof. In some embodiments, an epigenetic editor comprises a DNMT3A domain that lacks a regulatory subdomain and only maintains a catalytic domain. In some embodiments, the epigenetic editor comprising a DNMT3A catalytic domain effects methylation exclusively at CpG sequences. In some embodiments, an epigenetic editor comprising a DNMT3A domain that comprises a mutation, e.g. a R836A or R836Q mutation (numbering according to SEQ ID NO: 574), has higher methylation activity at CpA, CpC, and/or CpT sequences as compared to an epigenetic editor comprising a wildtype DNMT3A domain.

### C. Histone Modifiers

In some embodiments, an effector domain of an epigenetic editor herein mediates histone modification. Histone modifications play a structural and biochemical role in gene transcription, such as by formation or disruption of the nucleosome structure that binds to the histone and prevents gene transcription. Histone modifications may include, for example, acetylation, deacetylation, methylation, phosphorylation, ubiquitination, SUMOylation and the like, e.g., at their N-terminal ends (“histone tails”). These modifications maintain or specifically convert chromatin structure, thereby controlling responses such as gene expression, DNA replication, DNA repair, and the like, which occur on chromosomal DNA. Post-translational modification of histones is an epigenetic regulatory mechanism and is considered essential for the genetic regulation of eukaryotic cells. Recent studies have revealed that chromatin remodeling factors such as SWI/SNF, RSC, NURF, NRD, and the like, which facilitate transcription factor access to DNA by modifying the nucleosome structure; histone acetyltransferases (HATs) that regulate the acetylation state of histones; and histone deacetylases (HDACs), act as important regulators.

In particular, the unstructured N-termini of histones may be modified by acetylation, deacetylation, methylation, ubiquitylation, phosphorylation, SUMOylation, ribosylation, citrullination O-GlcNAcylation, crotonylation, or any combination thereof. For example, histone acetyltransferases (HATs) utilize acetyl-CoA as a cofactor and catalyze the transfer of an acetyl group to the epsilon amino group of the lysine side chains. This neutralizes the lysine's positive charge and weakens the interactions between histones and DNA, thus opening the chromosomes for transcription factors to bind and initiate transcription. Acetylation of K14 and K9 lysines of histone H3 by histone acetyltransferase enzymes may be linked to transcriptional competence in humans. Lysine acetylation may directly or indirectly create binding sites for chromatin-modifying enzymes that regulate transcriptional activation. On the other hand, histone methylation of lysine 9 of histone H3 may be associated with heterochromatin, or transcriptionally silent chromatin.

In certain embodiments, an effector domain of an epigenetic editor described herein comprises a histone methyltransferase domain. The effector domain may comprise, for example, a DOT1L domain, a SET domain, a SUV39H1 domain, a G9a/EHMT2 protein domain, an EZH1 domain, an EZH2 domain, a SETDB1 domain, or any combination thereof.

In particular embodiments, the effector domain comprises a histone-lysine-N-methyltransferase SETDB1 domain.

In some embodiments, the effector domain comprises a histone deacetylase protein domain. In certain embodiments, the effector domain comprises a HDAC family protein domain, for example, a HDAC1, HDAC3, HDAC5, HDAC7, or HDAC9 protein domain. In particular embodiments, the effector domain comprises a nucleosome remodeling and deacetylase complex (NURD), which removes acetyl groups from histones.

### D. Other Effector Domains

In some embodiments, the effector domain comprises a tripartite motif containing protein (TRIM28, TIF1-beta, or KAP1). In certain embodiments, the effector domain comprises one or more KAP1 proteins. A KAP1 protein in an epigenetic editor herein may form a complex with one or more other effector domains of the epigenetic editor or one or more proteins involved in modulation of gene expression in a cellular environment. For example, KAP1 may be recruited by a KRAB domain of a transcriptional repressor. A KAP1 protein domain may interact with or recruit one or more protein complexes that reduces or silences gene expression. In some embodiments, KAP1 interacts with or recruits a histone deacetylase protein, a histone-lysine methyltransferase protein, a chromatin remodeling protein, and/or a heterochromatin protein. For example, a KAP1 protein domain may interact with or recruit a heterochromatin protein 1 (HP1) protein, a SETDB1 protein, an HDAC protein, and/or a NuRD protein complex component. In some embodiments, a KAP1 protein domain interacts with or recruits a ZFP90 protein (e.g., isoform 2 of ZFP90), and/or a FOXP3 protein. An exemplary KAP1 amino acid sequence is shown in SEQ ID NO: 629.

In some embodiments, the effector domain comprises a protein domain that interacts with or is recruited by one or more DNA epigenetic marks. For example, the effector domain may comprise a methyl CpG binding protein 2 (MECP2) protein that interacts with methylated DNA nucleotides in the target gene (which may or may not be at a CpG island of the target gene). An MECP2 protein domain in an epigenetic editor described herein may induce condensed chromatin structure, thereby reducing or silencing expression of the target gene. In some embodiments, an MECP2 protein domain in an epigenetic editor described herein may interact with a histone deacetylase (e.g. HDAC), thereby repressing or silencing expression of the target gene. In some embodiments, an MECP2 protein domain in an epigenetic editor described herein may block access of a transcription factor or transcriptional activator to the target sequence, thereby repressing or silencing expression of the target gene. An exemplary MECP2 amino acid sequence is shown in SEQ ID NO: 630.

Also contemplated as effector domains for the epigenetic editors described herein are, e.g., a chromoshadow domain, a ubiquitin-2 like Rad60 SUMO-like (Rad60-SLD/SUMO) domain, a chromatin organization modifier domain (Chromo) domain, a Yaf2/RYBP C-terminal binding motif domain (YAF2_RYBP), a CBX family C-terminal motif domain (CBX7_C), a zinc finger C3HC4 type (RING finger) domain (ZF-C3HC4_2), a cytochrome b5 domain (Cyt-b5), a helix-loop-helix domain (HLH), a helix-hairpin-helix motif domain (e.g., HHH_3), a high mobility group box domain (HMG-box), a basic leucine zipper domain (e.g., bZIP_1 or bZIP_2), a Myb_DNA-binding domain, a homeodomain, a MYM-type zinc finger with FCS sequence domain (ZF-FCS), an interferon regulatory factor 2-binding protein zinc finger domain (IRF-2BP1_2), an SSX repressor domain (SSXRD), a B-box-type zinc finger domain (ZF-B_box), a CXXC zinc finger domain (ZF-CXXC), a regulator of chromosome condensation 1 domain (RCC1), an SRC homology 3 domain (SH39), a sterile alpha motif domain (SAM_1), a sterile alpha motif domain (SAM_2), a sterile alpha motif/Pointed domain (SAM_PNT), a Vestigial/Tondu family domain (Vg_Tdu), a LIM domain, an RNA recognition motif domain (RRM_1), a paired amphipathic helix domain (PAH), a proteasomal ATPase OB C-terminal domain (Prot_ATP_ID_OB), a nervy homology 2 domain (NHR2), a hinge domain of cleavage stimulation factor subunit 2 (CSTF2_hinge), a PPAR gamma N-terminal region domain (PPARgamma_N), a CDC48 N-terminal domain (CDC48_2), a WD40 repeat domain (WD40), a FipI motif domain (Fip 1), a PDZ domain (PDZ_6), a Von Willebrand factor type C domain (VWC), a NAB conserved region 1 domain (NCD1), an S1 RNA-binding domain (S1), an HNF3 C-terminal domain (HNF_C), a Tudor domain (Tudor_2), a histone-like transcription factor (CBF/NF-Y) and archaeal histone domain (CBFD_NFYB_HMF), a zinc finger protein domain (DUF3669), an EGF-like domain (cEGF), a GATA zinc finger domain (GATA), a TEA/ATTS domain (TEA), a phorbol esters/diacylglycerol binding domain (C1-1), polycomb-like MTF2 factor 2 domain (Mtf2_C), a transactivation domain of FOXO protein family (FOXO-TAD), a homeobox KN domain (Homeobox_KN), a BED zinc finger domain (ZF-BED), a zinc finger of C3HC4-type RING domain (ZF-C3HC4_4), a RAD51 interacting motif domain (RAD51_interact), a p55-binding region of a methyl-CpG-binding domain protein MBD (MBDa), a Notch domain, a Raf-like Ras-binding domain (RBD), a Spin/Ssty family domain (Spin-Ssty), a PHD finger domain (PHD_3), a Low-density lipoprotein receptor domain class A (Ldl_recept_a), a CS domain, a DM DNA-binding domain, a TET1 domain, a TET2 domain, a TET3 domain, a VP64 domain, an RTA domain, a P65 domain, and a QLQ domain.

In some embodiments, the effector domain is a protein domain comprising a YAF2_RYBP domain or homeodomain or any combination thereof. In certain embodiments, the homeodomain of the YAF2_RYBP domain is a PRD domain, an NKL domain, a HOXL domain, or a LIM domain. In particular embodiments, the YAF2_RYBP domain may comprise a 32 amino acid Yaf2/RYBP C-terminal binding motif domain (32 aa RYBP).

In some embodiments, the effector domain comprises a protein domain selected from a group consisting of SUMO3 domain, Chromo domain from M phase phosphoprotein 8 (MPP8), chromoshadow domain from Chromobox 1 (CBX1), and SAM_1/SPM domain from Scm Polycomb Group Protein Homolog 1 (SCMH1).

In some embodiments, the effector domain comprises an HNF3 C-terminal domain (HNF_C). The HNF_C domain may be from FOXA1 or FOXA2. In certain embodiments, the HNF_C domain comprises an EH1 (engrailed homology 1) motif.

In some embodiments, the effector domain may comprise an interferon regulatory factor 2-binding protein zinc finger domain (IRF-2BP1_2), a Cyt-b5 domain from DNA repair factor HERC2 E3 ligase, a variant SH3 domain (SH39) from Bridging Integrator 1 (BIN1), an HMG-box domain from transcription factor TOX or ZF-C3HC4_2 RING finger domain from the polycomb component PCGF2, a Chromodomain-helicase-DNA binding protein 3 (CHD3) domain, or a ZNF783 domain.

### IV. Epigenetic Editors

Provided herein are epigenetic editors (i.e., epigenetic editing systems) that direct epigenetic modification(s) to a target sequence in a gene of interest, e.g., using two or more DNA-binding zinc finger domains as described herein and one or more effector domains (e.g., epigenetic repressor domains) as described herein, in any combination. The DNA-binding zinc finger domains directs the effector domain to modify the target sequence, resulting in gene repression or silencing, or in gene activation, depending on the effector domain, and the repression or silencing, or activation may be durable and inheritable across cell generations. In some aspects, the epigenetic editors described herein can repress or silence, or activate, genes reversibly or irreversibly in cells. In some embodiments, an epigenetic editor provided herein increases DNA accessibility or availability for editing.

In some embodiments, the system causes durable silencing of one or more human genes. In some embodiments, durable silencing lasts at least 7 days, at least 14 days, at least 21 days, at least 28 days, at least 60 days, at least 90 days, at least 180 days, at least 240 days, at least 360 days, or longer. In some embodiments, durable silencing lasts 1 month to 1 year, 1 month to 18 months, 1 month to two years, or two years to four years. In some embodiments, durable silencing comprises maintaining at least 50%, at least 60%, at least 70%, at least 80%, at least 90%, at least 95% or at least 99% of the original level of gene silencing over a period of time. In some embodiments, durable silencing comprises maintaining at least 50%, at least 60%, at least 70%, at least 80%, at least 90%, at least 95% or at least 99% of the original level of gene silencing over at least 7 days, at least 14 days, at least 21 days, at least 28 days, at least 60 days, at least 90 days, at least 180 days, at least 240 days, at least 360 days, or longer.

In some embodiments, the system causes inheritable silencing of one or more human genes. In some embodiments, the system causes silencing that persists over 1, 2, 3, 4 or more cell generations.

In particular embodiments, an epigenetic editor described herein comprises one or more fusion proteins, each comprising (1) two or more DNA-binding zinc finger domain(s) and (2) one or more effector domain(s). The effector domains may be on one or more fusion proteins comprised by the epigenetic editor. For example, a single fusion protein may comprise all of the effector domains with the two or more DNA-binding zinc finger domains. A fusion protein described herein may further comprise one or more linkers (e.g., peptide linkers), detectable tags, nuclear localization signals (NLSs), or any combination thereof. As used herein, a “fusion protein” refers to a chimeric protein in which two or more coding sequences (e.g., for DNA-binding domain(s) and/or effector domain(s)) are covalently or non-covalently joined, directly or indirectly.

In some embodiments, an epigenetic editor described herein comprises 2, 3, 4, 5, 6, 7, 8, 9, 10, or more effector (e.g., repression/repressor) domains, which may be identical or different. In certain embodiments, two or more of said effector domains function synergistically. Combinations of effector domains may comprise DNA methylation domains, histone deacetylation domains, histone methylation domains, and/or scaffold domains that recruit any of the above. For example, an epigenetic editor described herein may comprise one or more transcriptional repressor domains (e.g., a KRAB domain such as KOX1, ZIM3, ZFP28, or ZN627 KRAB) in combination with one or more DNA methylation domains (e.g., a DNMT domain) and/or recruiter domain (e.g., a DNMT3L domain). Such an epigenetic editor may comprise, for instance, a KRAB domain, a DNMT3A domain, and a DNMT3L domain. In some embodiments, the epigenetic editor further comprises an additional effector domain (e.g., a KAP1, MECP2, HP1b, CBX8, CDYL2, TOX, TOX3, TOX4, EED, RBBP4, RCOR1, or SCML2 domain). In some embodiments, the additional effector domain is a CDYL2, TOX, TOX3, TOX4, or HP1a domain. For example, an epigenetic editor described herein may comprise a CDYL2 and/or a TOX domain in combination with a KRAB domain (e.g., a KOX1 KRAB domain).

### A. Linkers

A fusion protein as described herein may comprise one or more linkers that connect components of the epigenetic editor. A linker may be a peptide or non-peptide linker.

In some embodiments, one or more linkers utilized in an epigenetic editor provided herein is a peptide linker, i.e., a linker comprising a peptide moiety. A peptide linker can be any length applicable to the epigenetic editor fusion proteins described herein. In some embodiments, the linker can comprise a peptide between 1 and 200 (e.g., between 1 and 80) amino acids. In some embodiments, the linker comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. Longer or shorter linkers are also contemplated. In some embodiments, the peptide linker is 3, 4, 5, 6, 7, 8, 9, 10, 11, 12, 13, 14, 15, 16, 17, 18, 19, 20, 25, 30, 25, 40, 45, 50, 55, 60, 65, 70, 75, 80, 85, 90, 95, or 100 amino acids in length. For example, the peptide linker may be 4, 5, 16, 20, 24, 27, 32, 40, 64, 92, or 104 amino acids in length. The peptide linker may be a flexible or rigid linker. In particular embodiments, the peptide linker comprises the amino acid sequence of any one of SEQ ID NOs: 631-637 and 664-665 or a sequence at least 75%, 80%, 85%, 90%, 91%, 92%, 93%, 94%, 95%, 96%, 97%, 98%, or 99% identical thereto.

In certain embodiments, the peptide linker is an XTEN linker. Such a linker may comprise part of the XTEN sequence (Schellenberger et al., Nat Biotechnol (2009) 27(1):1186-90, incorporated herein by reference), an unstructured hydrophilic polypeptide consisting only of residues G, S, P, T, E, and A. The term “XTEN” as used herein refers to a recombinant peptide or polypeptide lacking hydrophobic amino acid residues. XTEN linkers typically are unstructured and comprise a limited set of natural amino acids. Fusion of XTEN to proteins alters its hydrodynamic properties and reduces the rate of clearance and degradation of the fusion protein. These XTEN fusion proteins are produced using recombinant technology, without the need for chemical modifications, and degraded by natural pathways. The XTEN linker may be, for example, 5, 10, 16, 20, 26, or 80 amino acids in length. In some embodiments, the XTEN linker is 16 amino acids in length. In some embodiments, the XTEN linker is 80 amino acids in length. In certain embodiments, the XTEN linker may be XTEN10, XTEN16, XTEN20, or XTEN80. In certain embodiments, the XTEN linker may comprise the amino acid sequence of any one of SEQ ID NOs: 638-643 or a sequence at least 75%, 80%, 85%, 90%, 91%, 92%, 93%, 94%, 95%, 96%, 97%, 98%, or 99% identical thereto. In particular embodiments, the XTEN linker comprises the amino acid sequence of SEQ ID NO: 638. In particular embodiments, the XTEN linker comprises the amino acid sequence of SEQ ID NO: 643.

In some embodiments, one or more linkers utilized in an epigenetic editor provided herein is a non-peptide linker. For example, the linker may be a carbon bond, a disulfide bond, or carbon-heteroatom bond. In certain embodiments, the linker is a carbon-nitrogen bond of an amide linkage. In certain embodiments, the linker is a cyclic or acyclic, substituted or unsubstituted, or branched or unbranched aliphatic or heteroaliphatic linker.

In some embodiments, one or more linkers utilized in an epigenetic editor provided herein is polymeric (e.g., polyethylene, polyethylene glycol, polyamide, polyester, etc.). The linker may comprise, for example, a monomer, dimer, or polymer of aminoalkanoic acid; an aminoalkanoic acid (e.g., glycine, ethanoic acid, alanine, beta-alanine, 3-aminopropanoic acid, 4-aminobutanoic acid, 5-pentanoic acid, etc.); a monomer, dimer, or polymer of aminohexanoic acid (Ahx); or a polyethylene glycol moiety (PEG); or an aryl or heteroaryl moiety. In certain embodiments, the linker may be based on a carbocyclic moiety (e.g., cyclopentane or cyclohexane) or a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, alkyl halides, aryl halides, acyl halides, and isothiocyanates.

Various linker lengths and flexibilities can be employed between any two components of an epigenetic editor (e.g., between an effector domain (e.g., a repressor domain) and a DNA-binding domain (e.g., a Cas9 domain), between a first effector domain and a second effector domain, etc.). The linkers may range from very flexible linkers, such as glycine/serine-rich linkers, to more rigid linkers, in order to achieve the optimal length for effector domain activity for the specific application. In some embodiments, the more flexible linkers are glycine/serine-rich linkers (GS-rich linkers), where more than 45% (e.g., more than 48, 50, 55, 60, 70, 80, or 90%) of the residues are glycine or serine residues. Non-limiting examples of the GS-rich linkers are (GGGGS)n (SEQ ID NO: 664), (G)n (SEQ ID NO: 703), and W linker (SEQ ID NO: 637). In some embodiments, the more rigid linkers are in the form of the form (EAAAK)n (SEQ ID NO: 665), (SGGS)n (SEQ ID NO: 704), and (XP)n (SEQ ID NO: 705). In the aforementioned formulae of flexible and rigid linkers, n may be any integer between 1 and 30. In some embodiments, n is 1, 2, 3, 4, 5, 6, 7, 8, 9, 10, 11, 12, 13, 14, or 15. In some embodiments, the linker comprises a (GGS)n (SEQ ID NO: 706) motif, wherein n is 1, 3, or 7. In some embodiments, the linker comprises a (GGGGS)n motif, wherein n is 4 (SEQ ID NO: 636).

In some embodiments, a linker in an epigenetic editor described herein comprises a nuclear localization signal, for example, with the amino acid sequence of any one of SEQ ID NOs: 644-649. In some embodiments, a linker in an epigenetic editor described herein comprises an expression tag, e.g., a detectable tag such as a green fluorescent protein.

### B. Nuclear Localization Signals

A fusion protein described herein may comprise one or more nuclear localization signals, and in certain embodiments, may comprise two or more nuclear localization signals. For example, the fusion protein may comprise 1, 2, 3, 4, 5, 6, 7, 8, 9, 10, or more nuclear localization signals. As used herein, a “nuclear localization signal” (NLS) is an amino acid sequence that directs proteins to the nucleus. In certain embodiments, the NLS may be an SV40 NLS (e.g., with the amino acid sequence of SEQ ID NO: 644). The fusion protein may comprise an NLS at its N-terminus, C-terminus, or both, and/or an NLS may be embedded in the middle of the fusion protein (e.g., at the N- or C-terminus of a DNA-binding domain or an effector domain).

In some embodiments, the fusion protein may comprise two NLSs. The fusion protein may comprise two NLSs at its N-terminus or C-terminus. The fusion protein may comprise one NLS located at its N-terminus and one NLS embedded in the middle of the fusion protein, or one NLS located at its C-terminus and one NLS embedded in the middle of the fusion protein. The fusion protein may comprise two NLSs embedded in the middle of the fusion protein.

In some embodiments, the fusion protein may comprise four NLSs. The fusion protein may comprise at least two (e.g., two, three, or four) NLSs at its N-terminus or C-terminus. The fusion protein may comprise at least one (e.g., one, two, three, or four) NLSs embedded in the middle of the fusion protein. In particular embodiments, the fusion protein may comprise two NLSs at its N-terminus and two NLSs at its C-terminus.

An NLS described herein may be an endogenous NLS sequence. In certain embodiments, an NLS described herein comprises the amino acid sequence of any one of SEQ ID NOs: 644-649, or a sequence at least 75%, 80%, 85%, 90%, 91%, 92%, 93%, 94%, 95%, 96%, 97%, 98%, or 99% identical to the selected sequence. In particular embodiments, the NLS comprises the amino acid sequence of SEQ ID NO: 644. Additional NLSs are known in the art.

In some embodiments, an epigenetic editor comprising a fusion protein that comprises at least one NLS at the N-terminus and at least one NLS at the C-terminus may increase the efficiency of the epigenetic editor by at least 5%, at least 10%, at least 15%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 5,000%, at least 10,000%, at least 50,000%, at least 100,000%, or more as compared to an epigenetic editor with a corresponding fusion protein that does not have at least one NLS at the N-terminus and at least one NLS at the C-terminus.

In some embodiments, an epigenetic editor comprising a fusion protein that comprises two NLSs at the N-terminus and two NLSs at the C-terminus may increase the efficiency of the epigenetic editor by at least 5%, at least 10%, at least 15%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 5,000%, at least 10,000%, at least 50,000%, at least 100,000%, or more as compared to an epigenetic editor with a corresponding fusion protein that does not have two NLSs at the N-terminus and two NLSs at the C-terminus.

### C. Tags

Epigenetic editors provided herein may comprise one or more additional sequences (“tags”) for tracking, detection, and localization of the editors. In some embodiments, the epigenetic editor comprises 1, 2, 3, 4, 5, 6, 7, 8, 9, 10 or more detectable tags. Each of the detectable tags may be the same or different.

For example, an epigenetic editor fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, poly-histidine tags (also referred to as histidine tags or His-tags), maltose binding protein (MBP)-tags, nus-tags, glutathione-S-transferase (GST)-tags, green fluorescent protein (GFP)-tags, thioredoxin-tags, S-tags, Softags (e.g., Softag 1 or Softag 3), strep-tags, biotin ligase tags, FlAsH tags, V5 tags, and SBP-tags. Additional suitable sequences will be apparent to those of skill in the art.

It will be appreciated that, where protein sequences are provided, protein sequences comprising a suitable tag are also embraced by the scope of this disclosure. Similarly, where protein sequences are provided that include a tag, tag-less protein sequences are also embraced.

### D. Fusion Protein Configurations

A fusion protein of an epigenetic editor described herein may have its components structured in different configurations. For example, a DNA-binding domain (comprising a single DNA-binding zinc finger domain) may be at the C-terminus, the N-terminus, or in between two or more epigenetic effector domains or additional domains. In some embodiments, the DNA-binding domain is at the C-terminus of the epigenetic editor. In some embodiments, the DNA-binding domain is at the N-terminus of the epigenetic editor. In some embodiments, the DNA-binding domain is linked to one or more nuclear localization signals. In some embodiments, the DNA-binding domain is flanked by an epigenetic effector domain and/or an additional domain on both sides. In some embodiments, where “DBD” indicates a single DNA-binding zinc finger domain and “ED” indicates effector domain, the epigenetic editor comprises the configuration of:

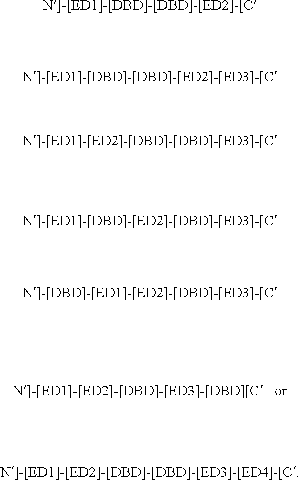

In some embodiments, an epigenetic editor comprises a DNA-binding domain (DBD, a single DNA-binding zinc finger domain), a DNA methyltransferase (DNMT) domain, and a transcriptional repressor (“repressor”) domain that represses or silences expression of a target gene. The DBD, DNMT, and transcriptional repressor domains may be any as described herein, in any combination. The DBD, DNMT domain, and repressor domain may be in any configuration, e.g., with any of said domains at the N-terminus, at the C-terminus, or in the middle of the fusion protein. In some embodiments, the epigenetic editor comprises a fusion protein with the configuration of:

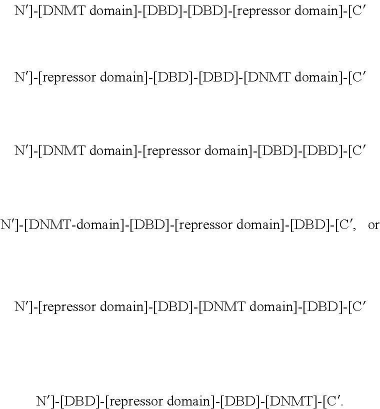

In some embodiments, a connecting structure “]-[” in any one of the epigenetic editor structures is a linker, e.g., a peptide linker; a detectable tag; a peptide bond; a nuclear localization signal; and/or a promoter or regulatory sequence. In an epigenetic editor structure, the multiple connecting structures “]-[” may be the same or may each be a different linker, tag, NLS, or peptide bond. In some embodiments, the DNMT domain may comprise any one of the domains in Table 3, or any combinations or homologs thereof. In particular embodiments, the DNMT domain comprises DNMT3A or a truncated version thereof, DNMT3L or a truncated version thereof, or both. As disclosed herein, the DBD is a ZFP domain. In certain embodiments, the repressor domain comprises any one of the domains shown in Table 1 or 2, or any combinations or homologs thereof. For example, the repressor domain may be a KRAB domain. In certain embodiments, the repressor domain is a ZFP28, ZN627, ZIM3, KOX1, KAP1, MeCP2, HP1b, CBX8, CDYL2, TOX, TOX3, TOX4, EED, RBBP4, RCOR1, or SCML2 domain, or a fusion of two of said domains (e.g., a fusion of the N- and C-terminal regions of ZIM3 and KOX1 KRAB). In particular embodiments, the repressor domain is a KRAB domain from ZFP28, ZN627, ZIM3, or KOX1.

In some embodiments, the epigenetic editor comprises a configuration selected from

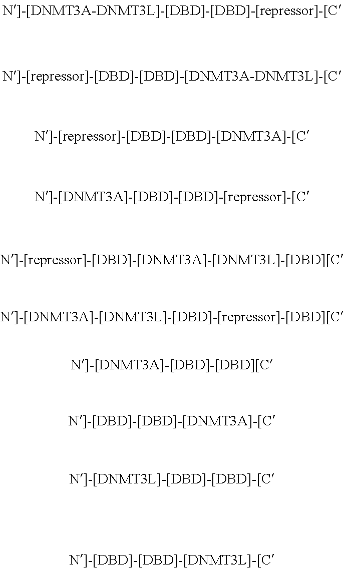

wherein [DNMT3A-DNMT3L] indicates that the DNMT3A and DNMT3L domains are directly fused via a peptide bond, and wherein the connecting structure]-[is any one of the linkers as described herein, a detectable tag, an affinity domain, a peptide bond, a nuclear localization signal, a promoter, and/or a regulatory sequence. The DBD, repressor, DNMT3A, and DNMT3L domains may be any as described herein, in any combination. For example, the DNMT3A and DNMT3L domains may be selected from those in Table 3. As disclosed herein, the DBD is a ZFP domain; the repressor domain is a KRAB domain derived from KOX1, ZIM3, ZFP28, or ZN627; the DNMT3A domain is a human DNMT3A domain; and the DNMT3L domain is a human or mouse DNMT3L domain; any combination of these components is also contemplated by the present disclosure.

In some embodiments, the epigenetic editor comprises a configuration selected from

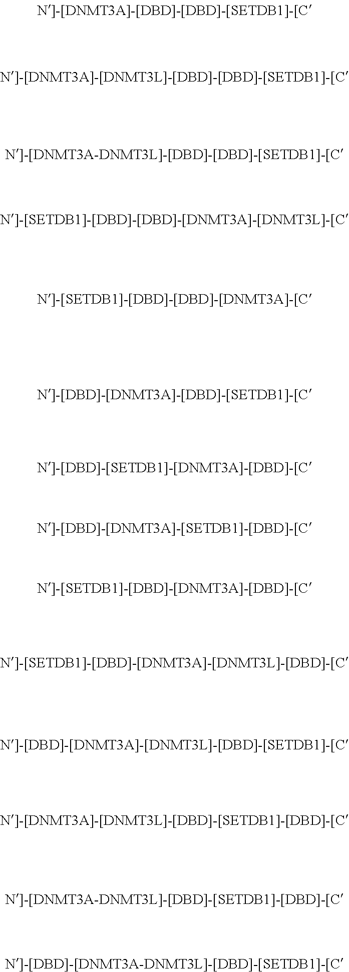

wherein [DNMT3A-DNMT3L] indicates that the DNMT3A and DNMT3L domains are directly fused via a peptide bond, and wherein the connecting structure]-[is any one of the linkers as described herein, a detectable tag, an affinity domain, a peptide bond, a nuclear localization signal, a promoter, and/or a regulatory sequence. The DBD, SETDB1, DNMT3A, and DNMT3L domains may be any as described herein, in any combination. As disclosed herein, the DBD is a ZFP domain; the SETDB1 domain is derived from human SETDB1, ZIM3, ZFP28, or ZN627; the DNMT3A domain is a human DNMT3A domain; and the DNMT3L domain is a human or mouse DNMT3L domain; any combination of these components is also contemplated by the present disclosure.

Particular constructs contemplated herein include: NLS-NLS-DNMT3A-DNMT3L-XTEN80-[DNA-binding ZFP domain 1]-XTEN16-[DNA-binding ZFP domain 2]-NLS-XTEN16-ZN627 KRAB-NLS-NLS (Configuration 1), NLS-NLS-DNMT3A-DNMT3L-XTEN80-[DNA-binding ZFP domain 1]-XTEN16-[DNA-binding ZFP domain 2]-NLS-XTEN16-ZN627 KRAB-NLS-NLS- (Configuration 2), NLS-NLS-ZN627 KRAB-XTEN16-[DNA-binding ZFP domain 1]-XTEN80-DNMT3A-DNMT3L-XTEN80-NLS-[DNA-binding ZFP domain 2]-XTEN16-ZN627 KRAB-NLS-NLS (Configuration 3),

The DNMT3L and DNMT3A may be derived from human parental proteins, mouse parental proteins, or any combination thereof. In certain embodiments, the DNMT3L and DNMT3A are derived from mouse and human parental proteins, respectively (mDNMT3L and hDNMT3A). In certain embodiments, the DNMT3L and DNMT3A are both derived from human parental proteins (hDNMT3L and hDNMT3A). In some embodiments, the KOX1 is human KOX1. Also contemplated is any of Configurations 1-6 wherein the KOX1 KRAB domain is replaced by a ZFP28, ZN627, or ZIM3 KRAB domain. In some embodiments, the ZFP28, ZN627, and ZIM3 are human ZFP28, ZN627, and ZIM3, respectively.

In particular embodiments, a fusion construct described herein may have Configuration 1 (2×NLS-hDNMT3A-W linker-hDNMT3L-XTEN80-DNA-binding zinc finger domain 1-XTEN16-DNA-binding zinc finger domain 1-NLS-XTEN16-ZN627KRAB-2×NLS) and comprise SEQ ID NO: 658, or a sequence at least 75%, 80%, 85%, 90%, 91%, 92%, 93%, 94%, 95%, 96%, 97%, 98%, or 99% identical thereto. In SEQ ID NO: 658 below, the XTEN linkers are underlined, the W linker is bolded, underlined, and italicized, the NLS sequences are bolded and underlined, the DNMT3A sequence is italicized, the DNMT3L sequence is underlined and italicized, the DNA-binding zinc finger domains are bolded, and the KRAB domain is underlined and bolded. Variable amino acids represented by Xs are the amino acids of the DNA-recognition helix of the zinc finger and XX in italics may be either TR, IR, TK, LR or LK:

(SEQ ID NO: 658)
MPKKKRKVPKKKRKVYNHDQEFDPPKVYPPVPAEKRKPIRVLSLF
DGIATGLLVLKDLGIQVDRYIASEVCEDSITVGMVRHQGKIMYVG
DVRSVTQKHIQEWGPFDLVIGGSPCNDLSIVNPARKGLYEGTGRL
FFEFYRLLHDARPKEGDDRPFFWLFENVVAMGVSDKRDISRFLES
NPVMIDAKEVSAAHRARYFWGNLPGMNRPLASTVNDKLELQECLE
HGRIAKFSKVRTITTRSNSIKQGKDQHFPVFMNEKEDILWCTEME
RVFGFPVHYTDVSNMSRLARQRLLGRSWSVPVIRHLFAPLKEYFA
CVSSGNSNANSRGPSFSSGLVPLSLRGSHMAAIPALDPEAEPSMD
VILVGSSELSSSVSPGTGRDLIAYEVKANQRNIEDICICCGSLQV
HTQHPLFEGGICAPCKDKFLDALFLYDDDGYQSYCSICCSGETLL
ICGNPDCTRCYCFECVDSLVGPGTSGKVHAMSNWVCYLCLPSSRS
GLLQRRRKWRSQLKAFYDRESENPLEMFETVPVWRRQPVRVLSLF
EDIKKELTSLGFLESGSDPGQLKHVVDVTDTVRKDVEEWGPFDLV
YGATPPLGHTCDRPPSWYLFQFHRLLQYARPKPGSPRPFFWMFVD
NLVLNKEDLDVASRFLEMEPVTIPDVHGGSLQNAVRVWSNIPAIR
SRHWALVSEEELSLLAQNKQSSKLAAKWPTKLVKNCFLPLREYFK
YFSTELTSSLGGPSSGAPPPSGGSPAGSPTSTEEGTSESATPESG
PGTSTEPSEGSAPGSPAGSPTSTEEGTSTEPSEGSAPGTSTEPSE
SRPGERPFQCRICMRNFSXXXXXXXHXXTHTGEKPFQCRICMRNF
SXXXXXXXHXXTH[linker]PFQCRICMRNFSXXXXXXXHXXTH
TGEKPFQCRICMRNFSXXXXXXXXXXTH[linker]PFQCRICMR
NFSXXXXXXXHXXTHTGEKPFQCRICMRNFSXXXXXXXHXXTHLR
GSGVDGSSGSETPGTSESATPESERPFQCRICMRNFSXXXXXXXH
XXTHTGEKPFACDICGRKFAXXXXXXXHXXIH[linker]PFQCR
ICMRNFSXXXXXXXHXXTHTGEKPFACDICGRKFAXXXXXXXHXX
IH[linker]PFQCRICMRNFSXXXXXXXHXXTHTGEKPFACDIC
GRKFAXXXXXXXHXXIHLRQKDAARGSSPKKKRKVGVDGSSGSET
PGTSESATPESTGDSVAFEDVAVNFTLEEWALLDPSQKNLYRDVM
RETFRNLASVGKQWEDQNIEDPFKIPRRNISHIPERLCESKEGGQ
GEESAPKKKRKVPKKKRKV

In particular embodiments, a fusion construct described herein may have Configuration 2 (2×NLS-ZN627KRAB-XTEN16-DNA-binding zinc finger domain 1-XTEN80-hDNMT3A-W linker-hDNMT3L-XTEN80-NLS-DNA-binding zinc finger domain 2-XTEN16-ZN627KRAB-2×NLS) and comprise SEQ ID NO: 659, or a sequence at least 75%, 80%, 85%, 90%, 91%, 92%, 93%, 94%, 95%, 96%, 97%, 98%, or 99% identical thereto. In SEQ ID NO: 659 below, the XTEN linkers are underlined, the W linker is bolded, underlined, and italicized, the NLS sequences are bolded and underlined, the DNMT3A sequence is italicized, the DNMT3L sequence is underlined and italicized, the DNA-binding zinc finger domains are bolded, and the KRAB domain is underlined and bolded. Variable amino acids represented by Xs are the amino acids of the DNA-recognition helix of the zinc finger and XX in italics may be either TR, IR, TK, LR or LK.

(SEQ ID NO: 659)
MPKKKRKVPKKKRKVDSVAFEDVAVNFTLEEWALLDPSQKNLYRD
VMRETFRNLASVGKQWEDQNIEDPFKIPRRNISHIPERLCESKEG
GQGEESASGSETPGTSESATPESTGERPFQCRICMRNFSXXXXXX
XHXXTHTGEKPFACDICGRKFAXXXXXXXHXXIH[linker]PFQ
CRICMRNFSXXXXXXXHXXTHTGEKPFACDICGRKFAXXXXXXXH
XXIH[linker]PFQCRICMRNFSXXXXXXXHXXTHTGEKPFACD
ICGRKFAXXXXXXXHXXIHLRQKDAARGSSPKKKRKVGVDGSGGP
SSGAPPPSGGSPAGSPTSTEEGTSESATPESGPGTSTEPSEGSAP
GSPAGSPTSTEEGTSTEPSEGSAPGTSTEPSEYNHDQEFDPPKVY
PPVPAEKRKPIRVLSLFDGIATGLLVLKDLGIQVDRYIASEVCED
SITVGMVRHQGKIMYVGDVRSVTQKHIQEWGPFDLVIGGSPCNDL
SIVNPARKGLYEGTGRLFFEFYRLLHDARPKEGDDRPFFWLFENV
VAMGVSDKRDISRFLESNPVMIDAKEVSAAHRARYFWGNLPGMNR
PLASTVNDKLELQECLEHGRIAKFSKVRTITTRSNSIKQGKDQHF
PVFMNEKEDILWCTEMERVFGFPVHYTDVSNMSRLARQRLLGRSW
SVPVIRHLFAPLKEYFACVSSGNSNANSRGPSFSSGLVPLSLRGS
HMAAIPALDPEAEPSMDVILVGSSELSSSVSPGTGRDLIAYEVKA
NQRNIEDICICCGSLQVHTQHPLFEGGICAPCKDKFLDALFLYDD
DGYQSYCSICCSGETLLICGNPDCTRCYCFECVDSLVGPGTSGKV
HAMSNWVCYLCLPSSRSGLLQRRRKWRSQLKAFYDRESENPLEMF
ETVPVWRRQPVRVLSLFEDIKKELTSLGFLESGSDPGQLKHVVDV
TDTVRKDVEEWGPFDLVYGATPPLGHTCDRPPSWYLFQFHRLLQY
ARPKPGSPRPFFWMFVDNLVLNKEDLDVASRFLEMEPVTIPDVHG
GSLQNAVRVWSNIPAIRSRHWALVSEEELSLLAQNKQSSKLAAKW
PTKLVKNCFLPLREYFKYFSTELTSSLGGPSSGAPPPSGGSPAGS
PTSTEEGTSESATPESGPGTSTEPSEGSAPGSPAGSPTSTEEGTS
TEPSEGSAPGTSTEPSESRPGERPFQCRICMRNFSXXXXXXXHXX
THTGEKPFQCRICMRNFSXXXXXXXHXXTH[linker]PFQCRIC
MRNFSXXXXXXXHXXTHTGEKPFQCRICMRNFSXXXXXXXHXXTH
[linker]PFQCRICMRNFSXXXXXXXHXXTHTGEKPFQCRICMR
NFSXXXXXXXHXXTHLRGSGVDGSSGSETPGTSESATPESDSVAF
EDVAVNFTLEEWALLDPSQKNLYRDVMRETERNLASVGKQWEDQN
IEDPFKIPRRNISHIPERLCESKEGGQGEESAPKKKRKVPKKKRK
V

In particular embodiments, a fusion construct described herein may have Configuration 3 (2×NLS-ZN627KRAB-XTEN16-NLS-DNA-binding zinc finger domain 1-XTEN16-DNA-binding zinc finger domain 2-XTEN80-hDNMT3A-Wlink-hDNMT3L-NLS-FLAG-2×NLS and comprise SEQ ID NO: 23 or a sequence at least 75%, 80%, 85%, 90%, 91%, 92%, 93%, 94%, 95%, 96%, 97%, 98%, or 99% identical thereto. In SEQ ID NO: 23 below, the XTEN linkers are underlined, the W linker is bolded, underlined, and italicized, the NLS sequences are bolded and underlined, the DNMT3A sequence is italicized, the DNMT3L sequence is underlined and italicized, the DNA-binding zinc finger domains are bolded, and the KRAB domain is underlined and bolded. Variable amino acids represented by Xs are the amino acids of the DNA-recognition helix of the zinc finger and XX in italics may be either TR, IR, TK, LR or LK.

(SEQ ID NO: 23)
MPKKKRKVPKKKRKVDSVAFEDVAVNFTLEEWALLDPSQKNLYRD
VMRETFRNLASVGKQWEDQNIEDPFKIPRRNISHIPERLCESKEG
GQGEESASGSETPGTSESATPESTGPKKKRKVSRPGERPFQCRIC
MRNFSXXXXXXXHXXTHTGEKPFQCRICMRNFSXXXXXXXHXXTH
[linker]PFQCRICMRNFSXXXXXXXHXXTHTGEKPFQCRICMR
NFSXXXXXXXHXXTH[linker]PFQCRICMRNFSXXXXXXXHXX
THTGEKPFQCRICMRNFSXXXXXXXHXXTHLRGSGVDGSSGSETP
GTSESATPESERPFQCRICMRNFSXXXXXXXHXXTHTGEKPFACD
ICGRKFAXXXXXXXHXXIH[linker]PFQCRICMRNFSXXXXXX
XXXXTHTGEKPFACDICGRKFAXXXXXXXHXXIH[linker]PFQ
CRICMRNFSXXXXXXXHXXTHTGEKPFACDICGRKFAXXXXXXXH
XXIHLRQKDAARGSSGVDGSGGPSSGAPPPSGGSPAGSPTSTEEG
TSESATPESGPGTSTEPSEGSAPGSPAGSPTSTEEGTSTEPSEGS
APGTSTEPSEYNHDQEFDPPKVYPPVPAEKRKPIRVLSLFDGIAT
GLLVLKDLGIQVDRYIASEVCEDSITVGMVRHQGKIMYVGDVRSV
TQKHIQEWGPFDLVIGGSPCNDLSIVNPARKGLYEGTGRLFFEFY
RLLHDARPKEGDDRPFFWLFENVVAMGVSDKRDISRFLESNPVMI
DAKEVSAAHRARYFWGNLPGMNRPLASTVNDKLELQECLEHGRIA
KFSKVRTITTRSNSIKQGKDQHFPVFMNEKEDILWCTEMERVFGF
PVHYTDVSNMSRLARQRLLGRSWSVPVIRHLFAPLKEYFACVSSG
NSNANSRGPSFSSGLVPLSLRGSHMAAIPALDPEAEPSMDVILVG
SSELSSSVSPGTGRDLIAYEVKANQRNIEDICICCGSLQVHTQHP
LFEGGICAPCKDKFLDALFLYDDDGYQSYCSICCSGETLLICGNP
DCTRCYCFECVDSLVGPGTSGKVHAMSNWVCYLCLPSSRSGLLQR
RRKWRSQLKAFYDRESENPLEMFETVPVWRRQPVRVLSLFEDIKK
ELTSLGFLESGSDPGQLKHVVDVTDTVRKDVEEWGPFDLVYGATP
PLGHTCDRPPSWYLFQFHRLLQYARPKPGSPRPFFWMFVDNLVLN
KEDLDVASRFLEMEPVTIPDVHGGSLQNAVRVWSNIPAIRSRHWA
LVSEEELSLLAQNKQSSKLAAKWPTKLVKNCFLPLREYFKYFSTE
LTSSLPKKKRKVPKKKRKV

In some embodiments, one or both linker sequences may be TGSQKP (SEQ ID NO: 651). In some embodiments, one or both linker sequences may be TGGGGSQKP (SEQ ID NO: 652). In some embodiments, one linker sequence may have the amino acid sequence of SEQ ID NO: 651 and the other linker sequence may have the amino acid sequence of SEQ ID NO: 652.

In some embodiments, a fusion construct described herein (e.g., the fusion construct of any one of Configurations 1-3) is within an expression construct that comprises a WPRE sequence, a polyadenylation site, or both. In certain embodiments, the WPRE sequence is in a 3′ noncoding region. In certain embodiments, the WPRE sequence is upstream from a poly-adenylation site. In particular embodiments, the expression construct comprises the fusion construct (e.g., of any one of Configurations 1-3 and a WPRE sequence in a 3′ noncoding region upstream from a polyadenylation site.

Multiple fusion proteins may be used to effect activation or repression of a target gene or multiple target genes. For example, an epigenetic editor fusion protein comprising a DNA-binding domain (e.g., a dCas9 domain) and an effector domain may be co-delivered with two or more guide polynucleotides (e.g., gRNAs), each targeting a different target DNA sequence. The target sites for two of the DNA-binding domains may be the same or in the vicinity of each other, or separated by, for example, about 100 base pairs, about 200 base pairs, about 300 base pairs, about 400 base pairs, about 500 base pairs, or about 600 or more base pairs. In addition, when targeting double-strand DNA, such as an endogenous gene locus, the guide polynucleotides may target the same or different strands (one or more to the positive strand and/or one or more to the negative strand).

### V. Target Sequences

An epigenetic editor herein may be directed to a target sequence in a target gene to effect epigenetic modification of the target gene. As used herein, a “target sequence,” a “target site,” or a “target region” is a nucleic acid sequence present in a gene of interest; in some instances, the target sequence may be outside but in the vicinity of the gene of interest wherein methylation or binding by a repressor of the target sequence represses expression of the gene. In some embodiments, the target sequence may be a hypomethylated or hypermethylated nucleic acid sequence.

The target sequence may be in any part of a target gene. In some embodiments, the target sequence is part of or near a noncoding sequence of the gene. In some embodiments, the target sequence is part of an exon of the gene. In some embodiments, the target sequence is part of or near a transcriptional regulatory sequence of the gene, such as a promoter or an enhancer. In some embodiments, the target sequence is adjacent to, overlaps with, or encompasses a CpG island. In certain embodiments, the target sequence is within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 base pairs (bp) flanking a target gene TSS. In certain embodiments, the target sequence is within 500 bp flanking the target gene TSS.

In some embodiments, where the DNA-binding domain of an epigenetic editor described herein is a zinc finger array, the target sequence may be recognized by said zinc finger array.

A target sequence described herein may be specific to one copy of a target gene, or may be specific to one allele of a target gene. Accordingly, the epigenetic modification and modulation of expression thereof may be specific to one copy or one allele of the target gene.

For example, an epigenetic editor may repress expression of a specific copy harboring a target sequence recognized by the DNA-binding domain (e.g., a copy associated with a disease or condition, or that harbors a mutation associated with a disease or condition).

### VI. Epigenetic Modifications

An epigenetic editor described herein may perform sequence-specific epigenetic modification(s) (e.g., alteration of chemical modification(s)) of a target gene that harbors the target sequence. Such epigenetic modulation may be safer and more easily reversible than modulation due to gene editing, e.g., with generation of DNA double-strand breaks. In some embodiments, the epigenetic modulation may reduce or silence the target gene. In some embodiments, the modification is at a specific site of the target sequence. In some embodiments, the modification is at a specific allele of the target gene. Accordingly, the epigenetic modification may result in modulated (e.g., reduced) expression of one copy of a target gene harboring a specific allele, and not the other copy of the target gene. In some embodiments, the specific allele is associated with a disease, condition, or disorder.

In some embodiments, the epigenetic modification reduces or abolishes transcription of the target gene harboring the target sequence. In some embodiments, the epigenetic modification reduces or abolishes transcription of a copy of the target gene harboring a specific allele recognized by the epigenetic editor. In some embodiments, the epigenetic editor reduces the level of or eliminates expression of a protein encoded by the target gene. In some embodiments, the epigenetic editor reduces the level of or eliminates expression of a protein encoded by a copy of the target gene harboring a specific allele recognized by the epigenetic editor. The target gene may be epigenetically modified in vitro, ex vivo, or in vivo.

The effector domain of an epigenetic editor described herein may alter (e.g., deposit or remove) a chemical modification at a nucleotide of the target gene or at a histone associated with the target gene. The chemical modification may be altered at a single nucleotide or a single histone, or may be altered at 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides.

In some embodiments, an effector domain of an epigenetic editor described herein may alter a CpG dinucleotide within the target gene. In some embodiments, all CpG dinucleotides within 2000, 1500, 1000, 500, or 200 bps flanking a target sequence (e.g., in an alteration site as described herein) are altered according to a modification type described herein, as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more of the CpG dinucleotides are altered as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the CpG dinucleotides are altered as compared to the original state of the gene or the gene in a comparable cell not contacted with the epigenetic editor. In some embodiments, one single CpG dinucleotide is altered, as compared to the original state of the gene or the gene in a comparable cell not contacted with the epigenetic editor.

An effector domain of an epigenetic editor described herein may alter a histone modification state of a histone associated with or bound to the target gene. For example, an effector domain may deposit a modification on one or more lysine residues of histone tails of histones associated with the target gene. In some embodiments, the effector domain may result in deacetylation of one or more histone tails of histones associated with the target gene, thereby reducing or silencing expression of the target gene. In some embodiments, the histone modification state is a methylation state. For example, the effector domain may result in a H3K9, H3K27 or H4K20 methylation (e.g. one or more of a H3K9me2, H3K9me3, H3K27me2, H3K27me3, and H4K20me3 methylation) at one or more histone tails associated with the target gene, thereby reducing or silencing expression of the target gene.

In some embodiments, all histone tails of histones bound to DNA nucleotides within 2000, 1500, 1000, 500, or 200 bps flanking the target sequence are altered according to a modification type as described herein, as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of the bound histones are altered as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of the bound histones are altered as compared to the original state of the chromosome or the chromosome in a comparable cell not contacted with the epigenetic editor. For example, one single histone tail of the bound histones may be altered as compared to the original state of the chromosome or the chromosome in a comparable cell not contacted with the epigenetic editor. As another example, one single bound histone octamer may be altered as compared to the original state of the chromosome or the chromosome in a comparable cell not contacted with the epigenetic editor.

The chemical modification deposited at target gene DNA nucleotides or histone residues may be at or in close proximity to a target sequence in the target gene. In some embodiments, an effector domain of an epigenetic editor described herein alters a chemical modification state of a nucleotide or histone tail bound to a nucleotide 100-200, 200-300, 300-400, 400-55, 500-600, 600-700, or 700-800 nucleotides 5′ or 3′ to the target sequence in the target gene. In some embodiments, an effector domain alters a chemical modification state of a nucleotide or histone tail bound to a nucleotide within 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 nucleotides flanking the target sequence. As used herein, “flanking” refers to nucleotide positions 5′ to the 5′ end of and 3′ to the 3′ end of a particular sequence, e.g. a target sequence.

In some embodiments, an effector domain mediates or induces a chemical modification change of a nucleotide or a histone tail bound to a nucleotide distant from a target sequence. Such modification may be initiated near the target sequence, and may subsequently spread to one or more nucleotides in the target gene distant from the target sequence. For example, an effector domain may initiate alteration of a chemical modification state of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration may spread to one or more nucleotides at least 100, 200, 300, 400, 500, 600, 700, 800, 900, 1000, 1100, 1200, 1300, 1400, 1500, 1600, 1700, 1800, 1900, 2000, 2500, 3000, or more nucleotides from the target sequence in the target gene, either upstream or downstream of the target sequence. In certain embodiments, the chemical modification may be initiated at less than 2, 3, 5, 10, 20, 30, 40, 50, or 100 nucleotides in the target gene and spread to at least 100, 200, 300, 400, 500, 600, 700, 800, 900, 1000, 2000, or more nucleotides in the target gene. In some embodiments, the chemical modification spreads to nucleotides in the entire target gene. Additional proteins or transcription factors, for example, transcription repressors, methyltransferases, or transcription regulation scaffold proteins, may be involved in the spreading of the chemical modification. Alternatively, the epigenetic editor alone may be involved.

In some embodiments, an epigenetic editor described herein reduces expression of a target gene by at least about 20%, at least about 30%, at least about 40%, at least about 50%, at least about 60%, at least about 70%, at least about 80%, at least about 90%, at least about 95%, at least about 99%, or more, as measured by transcription of the target gene in a cell, a tissue, or a subject as compared to a control cell, control tissue, or a control subject (e.g., in the absence of the epigenetic editor). In some embodiments, the epigenetic editors described herein reduces expression of a copy of target gene by at least about 20%, at least about 30%, at least about 40%, at least about 50%, at least about 60%, at least about 70%, at least about 80%, at least about 90%, at least about 95%, at least about 99%, or more, as measured by transcription of the copy of the target gene in a cell, a tissue, or a subject as compared to a control cell, control tissue, or a control subject. In certain embodiments, the copy of the target gene harbors a specific sequence or allele recognized by the epigenetic editor. In particular embodiments, the epigenetically modified copy encodes a functional protein, and accordingly an epigenetic editor disclosed herein may reduce or abolish expression and/or function of the protein. For example, an epigenetic editor described herein may reduce expression and/or function of a protein encoded by the target gene by at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, or at least 100 fold in a cell, a tissue, or a subject as compared to a control cell, control tissue, or a control subject.

Modulation of target gene expression can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP; changes in signal transduction; changes in phosphorylation and dephosphorylation; changes in receptor-ligand interactions; changes in concentrations of second messengers such as, for example, cGMP, cAMP, IP3, and Ca²⁺; changes in cell growth; changes in neovascularization; and/or changes in any functional effect of gene expression. Measurements can be made in vitro, in vivo, and/or ex vivo, and can be made by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays, changes in intracellular second messengers such as cGMP and inositol triphosphate (IP3), changes in intracellular calcium levels; cytokine release, and the like.

Methods for determining the expression level of a gene, for example the target of an epigenetic editor, may include, e.g., determining the transcript level of a gene by reverse transcription PCR, quantitative RT-PCR, droplet digital PCR (ddPCR), Northern blot, RNA sequencing, DNA sequencing (e.g., sequencing of complementary deoxyribonucleic acid (cDNA) obtained from RNA); next generation (Next-Gen) sequencing, nanopore sequencing, pyrosequencing, or Nanostring sequencing. Levels of protein expressed from a gene may be determined, e.g., by Western blotting, enzyme linked immuno-absorbance assays, mass-spectrometry, immunohistochemistry, or flow cytometry analysis. Gene expression product levels may be normalized to an internal standard such as total messenger ribonucleic acid (mRNA) or the expression level of a particular gene, e.g., a housekeeping gene.

In some embodiments, the effect of an epigenetic editor in modulating target gene expression may be examined using a reporter system. For example, an epigenetic editor may be designed to target a reporter gene encoding a reporter protein, such as a fluorescent protein. Expression of the reporter gene in such a model system may be monitored by, e.g., flow cytometry, fluorescence-activated cell sorting (FACS), or fluorescence microscopy. In some embodiments, a population of cells may be transfected with a vector that harbors a reporter gene. The vector may be constructed such that the reporter gene is expressed when the vector transfects a cell. Suitable reporter genes include genes encoding fluorescent proteins, for example green, yellow, cherry, cyan or orange fluorescent proteins. The population of cells carrying the reporter system may be transfected with DNA, mRNA, or vectors encoding the epigenetic editor targeting the reporter gene.

Suitable methods and assays for assessing protein activity in vitro and in vivo are well known to those of skill in the art, and exemplary suitable assays include, without limitation, assays measuring protein levels, e.g., via direct detection of a protein, e.g., via immunoassay (ELIZA assay, antibody-staining, or other antibody-based assay), via detection of protein activity, or via measuring the expression of a protein or by quantifying transcript levels of the respective protein-encoding gene, e.g., via RT-PCR assay, RNA-seq assay, microarray, northern blot, or other quantitative transcript assay. Some suitable assays are disclosed herein and other suitable assays will be apparent to the skilled artisan based on the present disclosure and the knowledge in the art.

### VII. Pharmaceutical Compositions

In one aspect, the present disclosure provides a pharmaceutical composition comprising as an active ingredient (or as the sole active ingredient) one or more epigenetic editors described herein or component(s) (e.g., fusion proteins and/or guide polynucleotides) thereof, or nucleic acid molecule(s) encoding said epigenetic editors or component(s) thereof.

For example, a pharmaceutical composition may comprise nucleic acid molecule(s) encoding the fusion protein(s) (and guide polynucleotides, where applicable) of an epigenetic editor described herein. In some embodiments, separate pharmaceutical compositions comprise the fusion protein(s) and the guide polynucleotide(s). A pharmaceutical composition may also comprise cells that have undergone epigenetic modification(s) mediated or induced by an epigenetic editor provided herein.

Generally, the epigenetic editors described herein or component(s) thereof, or nucleic acid molecule(s) encoding said epigenetic editors or component(s) thereof, of the present disclosure are suitable to be administered as a formulation in association with one or more pharmaceutically acceptable excipient(s), e.g., as described below.

The term “excipient” is used herein to describe any ingredient other than the compound(s) of the present disclosure. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives, or buffers, which enhance the shelf life or effectiveness of the antibody.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration.

### VIII. Delivery Methods

In some embodiments, the epigenetic editor or its component(s) are introduced to target cells in the form of nucleic acid molecule(s) encoding the epigenetic editor or its component(s); accordingly, the pharmaceutical compositions herein comprise the nucleic acid molecule(s). Such nucleic acid molecule(s) may be, for example, DNA, RNA or mRNA, and/or modified nucleic acid sequence(s) (e.g., with chemical modifications, a 5′ cap, or one or more 3′ modifications). In some embodiments, the nucleic acid molecule(s) may be delivered as naked DNA or RNA, for instance by means of transfection or electroporation, or can be conjugated to molecules (e.g., N-acetylgalactosamine) promoting uptake by target cells. In some embodiments, the nucleic acid molecule(s) may be in nucleic acid expression vector(s), which may include expression control sequences such as promoters, enhancers, transcription signal sequences, transcription termination sequences, introns, polyadenylation signals, Kozak consensus sequences, internal ribosome entry sites (IRES), etc. Such expression control sequences are well known in the art. A vector may also comprise a sequence encoding a signal peptide (e.g., for nuclear localization, nucleolar localization, or mitochondrial localization), associated with (e.g., inserted into or fused to) a sequence coding for a protein.

Examples of vectors include, but are not limited to, plasmid vectors; viral vectors based on vaccinia virus, poliovirus, adenovirus, adeno-associated virus, SV40, herpes simplex virus, human immunodeficiency virus, retrovirus (e.g., Murine Leukemia Virus, or spleen necrosis virus, vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and other recombinant vectors. In certain embodiments, the vector is a plasmid or a viral vector. Viral particles or virus-like particles (VLPs) may also be used to deliver nucleic acid molecule(s) encoding epigenetic editors or component(s) thereof as described herein. For example, “empty” viral particles can be assembled to contain any suitable cargo. Viral vectors and viral particles may also be engineered to incorporate targeting ligands to alter target tissue specificity.

In certain embodiments, an epigenetic editor as described herein or component(s) thereof are encoded by nucleic acid sequence(s) present in one or more viral vectors, or a suitable capsid protein of any viral vector. Examples of viral vectors include adeno-associated viral vectors (e.g., derived from AAV3, AAV3b, AAV4, AAV5, AAV6, AAV7, AAV8, AAV9, AAVrh8, AAV10, and/or variants thereof); retroviral vectors (e.g., Maloney murine leukemia virus, MML-V), adenoviral vectors (e.g., AD100), lentiviral vectors (e.g., HIV and FIV-based vectors), and herpesvirus vectors (e.g., HSV-2).

In some embodiments, delivery involves an adeno-associated virus (AAV) vector. AAV vector delivery may be particularly useful where the DNA-binding domain of an epigenetic editor fusion protein is a zinc finger array. Without wishing to be bound by any theory, the smaller size of zinc finger arrays compared to larger DNA-binding domains such as Cas protein domains may allow such a fusion protein to be conveniently packed in viral vectors such as an AAV vector.

Any AAV serotype, e.g., human AAV serotype, can be used for an AAV vector as described herein, including, but not limited to, AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), and AAV serotype 11 (AAV11), as well as variants thereof. In some embodiments, an AAV variant has at least 90%, 91%, 92%, 93%, 94%, 95%, 96%, 97%, 98%, or 99% amino acid sequence identity to a wildtype AAV. In certain embodiments, the AAV variant may be engineered such that its capsid proteins have reduced immunogenicity or enhanced transduction ability in humans. In some instances, one or more regions of at least two different AAV serotype viruses are shuffled and reassembled to generate a chimeric variant. For example, a chimeric AAV may comprise inverted terminal repeats (ITRs) that are of a heterologous serotype compared to the serotype of the capsid. The resulting chimeric AAV can have a different antigenic reactivity or recognition compared to its parental serotypes. In some embodiments, a chimeric variant of an AAV includes amino acid sequences from 2, 3, 4, 5, or more different AAV serotypes.

Non-viral systems are also contemplated for delivery as described herein. Non-viral systems include, but are not limited to, nucleic acid transfection methods including electroporation, sonoporation, calcium phosphate transfection, microinjection, DNA biolistics, lipid-mediated transfection, transfection through heat shock, compacted DNA-mediated transfection, lipofection, cationic agent-mediated transfection, and transfection with liposomes, immunoliposomes, exosomes, or cationic facial amphiphiles (CFAs). In certain embodiments, one or more mRNAs encoding epigenetic editor fusion proteins as described herein may be co-electroporated with one or more guide polynucleotides (e.g., gRNAs) as described herein. One important category of non-viral nucleic acid vectors is nanoparticles, which can be organic (e.g., lipid) or inorganic (e.g., gold). For instance, organic (e.g. lipid and/or polymer) nanoparticles can be suitable for use as delivery vehicles in certain embodiments of this disclosure.

In some embodiments, delivery is accomplished using a lipid nanoparticle (LNP).

LNP compositions are typically sized on the order of micrometers or smaller and may include a lipid bilayer. In some embodiments, a LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 25-100 nm, 35-75 nm, or 25-60 nm. Nanoparticle compositions encompass lipid nanoparticles (LNPs), liposomes (e.g., lipid vesicles), and lipoplexes.

An LNP as described herein may be made from cationic, anionic, or neutral lipids. In some embodiments, an LNP may comprise neutral lipids, such as the fusogenic phospholipid 1,2-dioleoyl-sn-glycero-3-phosphoethanolamine (DOPE) or the membrane component cholesterol, as helper lipids to enhance transfection activity and nanoparticle stability. In some embodiments, an LNP may comprise hydrophobic lipids, hydrophilic lipids, or both hydrophobic and hydrophilic lipids. Any lipid or combination of lipids that are known in the art can be used to produce an LNP. The lipids may be combined in any molar ratios to produce the LNP. In some embodiments, the LNP is a liver-targeting (e.g., preferentially or specifically targeting the liver) LNP. Exemplary suitable LNP formulations are disclosed herein and additional suitable LNP formulations will be apparent to the skilled artisan based on the present disclosure and the knowledge in the art, including, without limitation, LNP formulations disclosed in Hou et al., Lipid nanoparticles for mRNA delivery. Nat Rev Mater 6:1078-1094 (2021), the entire contents of which are incorporated herein by reference.

Any type of cell may be targeted for delivery of an epigenetic editor or component(s) thereof as described herein. For example, the cells may be eukaryotic or prokaryotic. In some embodiments, the cells are mammalian (e.g., human) cells. Human cells may include, for example, hepatocytes, biliary epithelial cells (cholangiocytes), stellate cells, Kupffer cells, and liver sinusoidal endothelial cells.

In some embodiments, an epigenetic editor described herein, or component(s) thereof, are delivered to a host cell for transient expression, e.g., via a transient expression vector. Transient expression of the epigenetic editor or its component(s) may result in prolonged or permanent epigenetic modification of the target gene. For example, the epigenetic modification may be stable for at least 1, 2, 3, 4, 5, 6, 7, 8, 9, 10. 11, or 12 weeks or more; or 3, 4, 5, 6, 7, 8, 9, 10, 11, or 12 months or more, after introduction of the epigenetic editor into the host cell. The epigenetic modification may be maintained after one or more mitotic and/or meiotic events of the host cell. In particular embodiments, the epigenetic modification is maintained across generations in offspring generated or derived from the host cell.

### IX. Therapeutic Uses of Epigenetic Editors

The present disclosure also provides methods for treating or preventing a condition in a subject, comprising administering to the subject an epigenetic editor or pharmaceutical composition as described herein. The epigenetic editor may effect an epigenetic modification of a target polynucleotide sequence in a target gene associated with a disease, condition, or disorder in the subject, thereby modulating expression of the target gene to treat or prevent the disease, condition, or disorder. In some embodiments, the epigenetic editor reduces the expression of the target gene to an extent sufficient to achieve a desired effect, e.g., a therapeutically relevant effect such as the prevention or treatment of the disease, condition, or disorder.

In some embodiments, a subject is administered a system for modulating (e.g., repressing) expression of a target gene, wherein the system comprises (1) the fusion protein(s) and, where relevant, guide polynucleotide(s) of an epigenetic editor as described herein, or (2) nucleic acid molecules encoding said fusion protein(s) and, where relevant, guide polynucleotide(s).

“Treat,” “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. In some embodiments, as compared with an equivalent untreated control, alleviating a symptom may involve reduction of the symptom by at least 3%, 5%, 10%, 20%, 40%, 50%, 60%, 80%, 90%, 95%, 98%, 99%, 99.5%, 99.9%, or 100% as measured by any standard technique.

In some embodiments, the subject may be a mammal, e.g., a human. In some embodiments, the subject is selected from a non-human primate such as chimpanzee, cynomolgus monkey, or macaque, and other ape and monkey species.

In some embodiments, a patient to be treated with an epigenetic editor of the present disclosure has received prior treatment for the condition to be treated.

An epigenetic editor of the present disclosure may be administered in a therapeutically effective amount to a patient with a condition described herein. “Therapeutically effective amount,” as used herein, refers to an amount of the therapeutic agent being administered that will relieve to some extent one or more of the symptoms of the disorder being treated, and/or result in clinical endpoint(s) desired by healthcare professionals. An effective amount for therapy may be measured by its ability to stabilize disease progression and/or ameliorate symptoms in a patient, and preferably to reverse disease progression. The ability of an epigenetic editor of the present disclosure to reduce or silence target gene expression may be evaluated by in vitro assays, e.g., as described herein, as well as in suitable animal models that are predictive of the efficacy in humans. Suitable dosage regimens will be selected in order to provide an optimum therapeutic response in each particular situation, for example, administered as a single bolus or as a continuous infusion, and with possible adjustment of the dosage as indicated by the exigencies of each case.

An epigenetic editor of the present disclosure may be administered without additional therapeutic treatments, i.e., as a stand-alone therapy (monotherapy). In some embodiments, the additional therapeutic is an antagonist, e.g., an inhibitor. In some embodiments, the additional therapeutic is an epigenetic editor targeting a target gene.

The epigenetic editors or components thereof (or nucleic acid molecules encoding the epigenetic editors or components thereof) of the present disclosure may be administered by any method accepted in the art, e.g., subcutaneously, intradermally, intratumorally, intranodally, intramuscularly, intravenously, intralymphatically, or intraperitoneally. In particular embodiments, a pharmaceutical composition of the present disclosure is administered intravenously to the subject.

### X. Definitions

The term “nucleic acid” as used herein refers to any oligonucleotide or polynucleotide containing nucleotides (e.g., deoxyribonucleotides or ribonucleotides) in either single- or double-strand form, and includes DNA and RNA. “Nucleotides” contain a sugar deoxyribose (DNA) or ribose (RNA), a base, and a phosphate group, and are linked together through the phosphate groups. “Bases” include purines and pyrimidines, which include natural compounds such as adenine, thymine, guanine, cytosine, uracil, inosine, and natural analogs; as well as synthetic derivatives of purines and pyrimidines, which include, but are not limited to, modified versions which place new reactive groups such as amines, alcohols, thiols, carboxylates, alkylhalides, etc. Nucleic acids may contain known nucleotide analogs and/or modified backbone residues or linkages, which may be synthetic, naturally occurring, and non-naturally occurring. Such nucleotide analogs, modified residues, and modified linkages are well known in the art, and may provide a nucleic acid molecule with enhanced cellular uptake, reduced immunogenicity, and/or increased stability in the presence of nucleases.

As used herein, an “isolated” or “purified” nucleic acid molecule is a nucleic acid molecule that exists apart from its native environment. For example, an “isolated” or “purified” nucleic acid molecule (1) has been separated away from the nucleic acids of the genomic DNA or cellular RNA of its source of origin; and/or (2) does not occur in nature. In some embodiments, an “isolated” or “purified” nucleic acid molecule is a recombinant nucleic acid molecule.

It will be understood that in addition to the specific proteins and nucleic acid molecules mentioned herein, the present disclosure also contemplates the use of variants, derivatives, homologs, and fragments thereof. A variant of any given sequence may have the specific sequence of residues (whether amino acid or nucleic acid residues) modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring sequence (in some embodiments, no more than 1, 2, 3, 4, 5, 6, 7, 8, 9, 10, 15, or 20 residues). For specific proteins described herein (e.g., KRAB, dCas9, DNMT3A, and DNMT3L proteins described herein), the present disclosure also contemplates any of the protein's naturally occurring forms, or variants or homologs that retain at least one of its endogenous functions (e.g., at least 50%, 60%, 70%, 80%, 90%, 85%, 96%, 97%, 98%, or 99% of its function as compared to the specific protein described).

As used herein, a homologue of any polypeptide or nucleic acid sequence contemplated herein includes sequences having a certain homology with the wildtype amino acid and nucleic sequence. A homologous sequence may include a sequence, e.g. an amino acid sequence which may be at least 50%, 55%, 65%, 75%, 85%, 90%, 91%, 92%<93%, 94%, 95%, 96%, 97%, 98%, or 99% identical to the subject sequence. The term “percent identical” in the context of amino acid or nucleotide sequences refers to the percent of residues in two sequences that are the same when aligned for maximum correspondence. In some embodiments, the length of a reference sequence aligned for comparison purposes is at least 30%, (e.g., at least 40, 50, 60, 70, 80, or 90%, or 100%) of the reference sequence. Sequence identity may be measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e-3 and e-100 indicating a closely related sequence.

The percent identity of two nucleotide or polypeptide sequences is determined by, e.g., BLAST® using default parameters (available at the U.S. National Library of Medicine's National Center for Biotechnology Information website). In some embodiments, the length of a reference sequence aligned for comparison purposes is at least 30%, (e.g., at least 40, 50, 60, 70, 80, or 90%) of the reference sequence.

It will be understood that the numbering of the specific positions or residues in polypeptide sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

The term “modulate” or “alter” refers to a change in the quantity, degree, or extent of a function. For example, an epigenetic editor as described herein may modulate the activity of a promoter sequence by binding to a motif within the promoter, thereby inducing, enhancing, or suppressing transcription of a gene operatively linked to the promoter sequence. As other examples, an epigenetic editor as described herein may block RNA polymerase from transcribing a gene, or may inhibit translation of an mRNA transcript. The terms “inhibit,” “repress,” “suppress,” “silence” and the like, when used in reference to an epigenetic editor or a component thereof as described herein, refers to decreasing or preventing the activity (e.g., transcription) of a nucleic acid sequence (e.g., a target gene) or protein relative to the activity of the nucleic acid sequence or protein in the absence of the epigenetic editor or component thereof. The term may include partially or totally blocking activity, or preventing or delaying activity. The inhibited activity may be, e.g., 10%, 20%, 30%, 40%, 50%, 60%, 70%, 75%, 80%, 85%, 90%, 91%, 92%, 93%, 94%, 95%, 96%, 97%, 98%, or 99% less than that of a control, or may be, e.g., at least 1.5-fold, 2-fold, 3-fold, 4-fold, 5-fold, or 10-fold less than that of a control.

The term “about” or “approximately” means within an acceptable error range for the particular value as determined by one of ordinary skill in the art, which will depend in part on how the value is measured or determined, e.g., the limitations of the measurement system. For example, “about” can mean within one or more than one standard deviation, per the practice in the given value. Where particular values are described in the application and claims, unless otherwise stated, the term “about” should be assumed to mean an acceptable error range for the particular value.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. Unless otherwise indicated, the recitation of a listing of elements herein includes any of the elements singly or in any combination. The recitation of an embodiment herein includes that embodiment as a single embodiment, or in combination with any other embodiment(s) herein. All publications, patents, patent applications, and other references mentioned herein are incorporated by reference in their entirety. To the extent that references incorporated by reference contradict the disclosure contained in the specification, the specification is intended to supersede and/or take precedence over any such contradictory material. Although a number of documents are cited herein, this citation does not constitute an admission that any of these documents forms part of the common general knowledge in the art.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that the present disclosure may be better understood, the following examples are set forth. These examples are for purposes of illustration only and are not to be construed as limiting the scope of the present disclosure in any manner.

### EXAMPLES

### Example 1. Design and Evaluation of Exemplary Epigenetic Editor Constructs

Several fusion protein constructs comprising two different DNA-binding zinc finger domains binding PCSK9 (ZFP152) and CLTA (ZFP2244) were developed to have epigenetic editing (here: silencing) activity. Epigenetic editors included a Dnmt3A domain and a Dnmt3L domain, linked by a W linker sequence and a KRAB domain from ZN627. Constructs further comprised nuclear localization signals, various linkers (e.g., XTEN 80, XTEN16), and were designed with FLAG tags.

Three conformations of multi-targeting epigenetic editors were created:
Conformation 1: PCSK9/CLTA ZF-OFF, an epigenetic editor comprising, from N- to C-terminus, a DNMT3A domain, a DNMT3L domain, a ZFP152 DNA-binding zinc finger domain, a ZFP2244 DNA-binding zinc finger domain, and a ZN627 KRAB domain. See FIG. 2, upper schematic.
Conformation 2: CLTA/PCSK9 ZF-OFF, an epigenetic editor comprising, from N- to C-terminus, a DNMT3A domain, a DNMT3L domain, a ZFP2244 DNA-binding zinc finger domain, a ZFP152 DNA-binding zinc finger domain, and a ZN627 KRAB domain. See FIG. 2, middle schematic.
Conformation 3: Palindromic CLTA/PCSK9 ZF-OFF, an epigenetic editor, comprising, from N-terminus to C-terminus, a ZN627 KRAB domain, a ZFP2244 DNA-binding zinc finger domain, a DNMT3A domain, a DNMT3L domain, a ZFP152 DNA-binding zinc finger domain, and a ZN627 KRAB domain. See FIG. 2, lower schematic.

Amino acid and encoding nucleotide sequences are provided below.

Silencing of the epigenetic editor constructs was evaluated in reporter cells, harboring, stably integrated into the respective endogenous genomic locus, either a TdTomato reporter sequence under the control of the endogenous PCSK9 promoter, or a GFP reporter sequence under the control of the endogenous CLTA promoter. Constructs (Conformations 1, 2, or 3) and controls were delivered to the respective reporter cells (HeLa PCSK9-TdTomato or GripTite® 293 CLTA-GFP cells, respectively) using standard procedures. Reporter expression was determined by standard FACS analysis and expressed as percentage of cells expressing TdTomato or GFP, respectively.

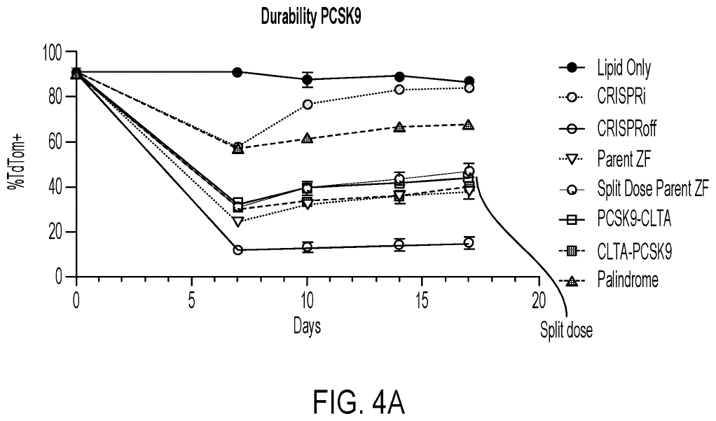

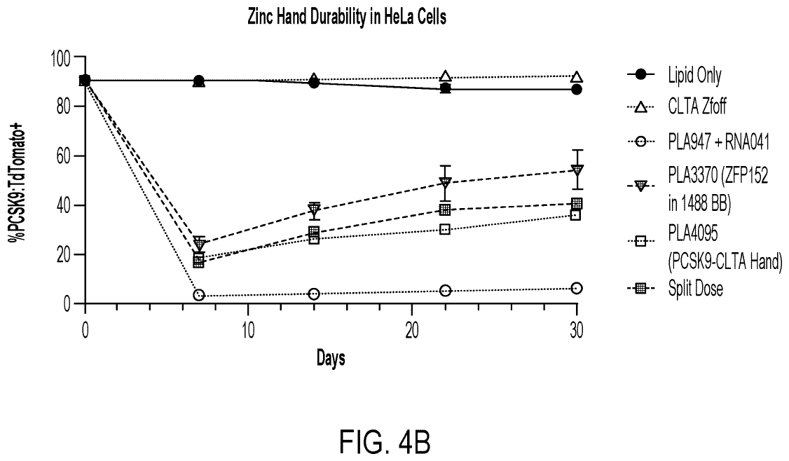

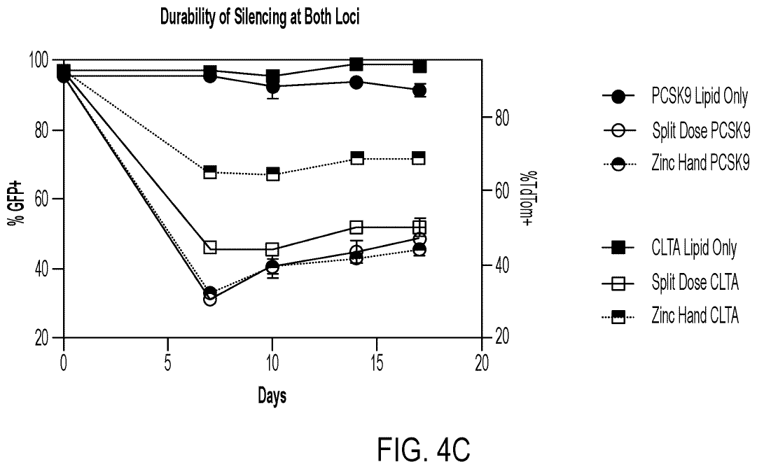

Longer term durability and time course of the observed silencing effect was evaluated by measuring reporter expression via FACS analysis over a time period of 17 days (FIGS. 4A-4C). Silencing levels observed at day 7 were stably maintained throughout the observation period for all dual-targeting epigenetic editors.

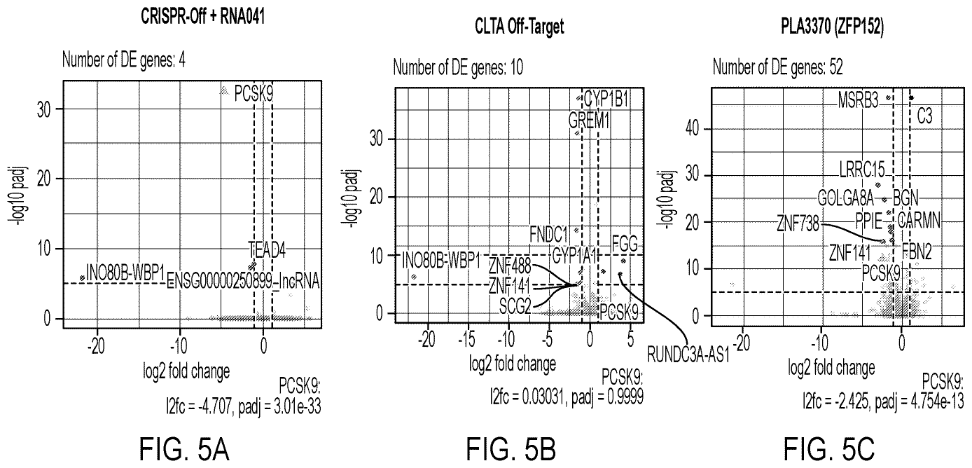

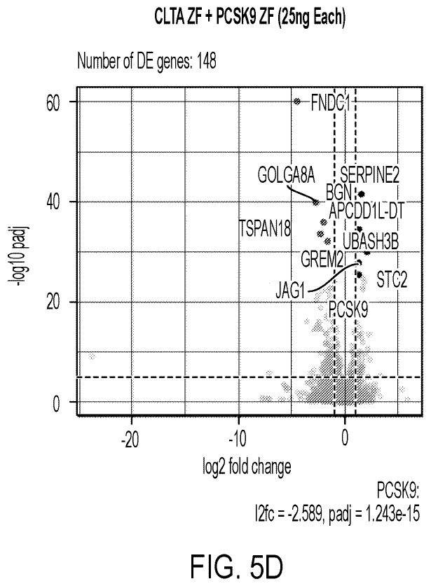

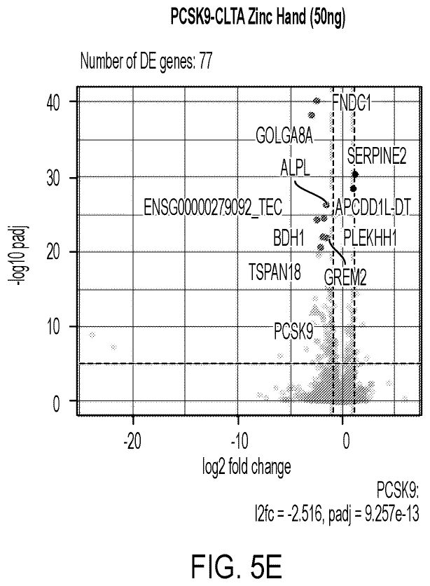

In order to evaluate specificity of the dual-targeting epigenetic editors, gene expression of treated cells was assessed by RNAseq at 14 days post treatment with the respective epigenetic editor and compared to expression levels observed in untreated control cells (FIGS. 5A-5E). Specificity was also assessed for a control CRISPR-OFF construct using a PCSK9-specific gRNA (FIG. 5A). FIG. 5B shows differential expression by the single PCSK9 ZF-OFF construct (comprising a ZFP152 PCSK9 DNA-binding zinc finger domain) at a 50 ng dose. FIG. 5C shows differential expression by the single CLTA ZF-OFF construct (comprising a ZFP2244 DNA-binding zinc finger domain) at a 50 ng dose. FIG. 5D shows differential expression by cells treated with a single 50 ng dose containing two single zinc finger constructs, wherein one construct targets CLTA (comprising a ZFP2244 CLTA DNA-binding zinc finger domain) and the other construct targets PCSK9 (comprising a ZFP152 PCSK9 DNA-binding zinc finger domain). The two constructs were administered in a 1:1 ratio, i.e., at 25 ng each. The number and identity of the differentially expressed genes observed was similar to the combination of the RNAseq profiles obtained from each construct alone (FIGS. 5B-C). FIG. 5E shows differential expression of PCSK9:TdTomato cells treated with a 50 ng dose of a single epigenetic editor comprising two DNA-binding zinc finger domains (Conformation 1). The RNAseq profile looks comparable to that of cells treated with two distinct zinc finger constructs (FIG. 5D) indicating that combining two or more DNA-binding zinc finger domains in a single epigenetic editor does not result in significant variations in specificity as compared to the specificity profiles of a combination of a plurality of epigenetic editors each targeting a single target.

The fusion protein constructs of the epigenetic editors used in this example are provided below:

TABLE 4
Fusion Protein Constructs

| | |
|---|---|
| Fusion protein 1 | MPKKKRKVPKKKRKVYNHDQEFDPPKVYPPVPAEKRKPIRVLSLFDGIATGLLVLKDLGIQV |
| Amino Acid Sequence | DRYIASEVCEDSITVGMVRHQGKIMYVGDVRSVTQKHIQEWGPFDLVIGGSPCNDLSIVNPA |
| PCSK9/CLTA ZF-OFF | RKGLYEGTGRLFFEFYRLLHDARPKEGDDRPFFWLFENVVAMGVSDKRDISRFLESNPVMID |
| 2xNLS-hDNMT3A-W | AKEVSAAHRARYFWGNLPGMNRPLASTVNDKLELQECLEHGRIAKFSKVRTITTRSNSIKQG |
| linker-hDNMT3L- | KDQHFPVFMNEKEDILWCTEMERVFGFPVHYTDVSNMSRLARQRLLGRSWSVPVIRHLFAPL |
| XTEN80-ZFP152- | KEYFACVSSGNSNANSRGPSFSSGLVPLSLRGSHMAAIPALDPEAEPSMDVILVGSSELSSS |
| XTEN16-ZFP2244-NLS- | VSPGTGRDLIAYEVKANQRNIEDICICCGSLQVHTQHPLFEGGICAPCKDKFLDALFLYDDD |
| XTEN16-ZN627KRAB- | GYQSYCSICCSGETLLICGNPDCTRCYCFECVDSLVGPGTSGKVHAMSNWVCYLCLPSSRSG |
| FLAG-2xNLS | LLQRRRKWRSQLKAFYDRESENPLEMFETVPVWRRQPVRVLSLFEDIKKELTSLGFLESGSD |
| SEQ ID NO: 17 | PGQLKHVVDVTDTVRKDVEEWGPFDLVYGATPPLGHTCDRPPSWYLFQFHRLLQYARPKPGS |
| | PRPFFWMFVDNLVLNKEDLDVASRFLEMEPVTIPDVHGGSLQNAVRVWSNIPAIRSRHWALV |
| | SEEELSLLAQNKQSSKLAAKWPTKLVKNCFLPLREYFKYFSTELTSSLGGPSSGAPPPSGGS |
| | PAGSPTSTEEGTSESATPESGPGTSTEPSEGSAPGSPAGSPTSTEEGTSTEPSEGSAPGTST |
| | EPSESRPGERPFQCRICMRNFSTPSKLDRHTRTHTGEKPFQCRICMRNFSLAENLRRHLRTH |
| | TGSQKPFQCRICMRNFSRQDNLGRHLRTHTGEKPFQCRICMRNFSDGGNLGRHLKTHTGGGG |
| | SQKPFQCRICMRNFSQSPHLKRHLRTHTGEKPFQCRICMRNFSQSTSLQRHLKTHLRGSGVD |
| | GSSGSETPGTSESATPESERPFQCRICMRNFSHKQHRDAHIRTHTGEKPFACDICGRKFARS |
| | ANLTRHTKIHTGSQKPFQCRICMRNFSRSDNLSEHIRTHTGEKPFACDICGRKFATSANLSR |
| | HTKIHTGSQKPFQCRICMRNFSIRSTLRDHIRTHTGEKPFACDICGRKFARTPVRMGHTKIH |
| | LRQKDAARGSSPKKKRKVGVDGSSGSETPGTSESATPESTGDSVAFEDVAVNFTLEEWALLD |
| | PSQKNLYRDVMRETFRNLASVGKQWEDQNIEDPFKIPRRNISHIPERLCESKEGGQGEESAD |
| | YKDDDDKAPKKKRKVPKKKRKV |
| Fusion protein 1 | ATGCCAAAAAAGAAGAGAAAGGTACCGAAGAAAAAAAGAAAGGTATACAATCACGATCAGGA |
| DNA sequence | GTTCGACCCCCCTAAGGTGTACCCACCAGTGCCTGCAGAGAAGAGGAAGCCAATCCGGGTGC |
| SEQ ID NO. 18 | TGAGCCTGTTTGATGGCATCGCCACCGGCCTGCTGGTGCTGAAGGATCTGGGCATCCAGGTG |
| | GACCGGTACATCGCCTCCGAGGTGTGCGAGGATTCTATCACCGTGGGCATGGTGCGCCACCA |
| | GGGCAAGATCATGTATGTGGGCGACGTGCGGTCCGTGACACAGAAGCACATCCAGGAGTGGG |
| | GCCCATTCGATCTGGTGATCGGCGGCAGCCCCTGTAATGACCTGTCCATCGTGAACCCTGCA |
| | AGGAAGGGACTGTACGAGGGAACCGGCCGGCTGTTCTTTGAGTTTTATAGACTGCTGCACGA |
| | CGCCAGGCCTAAGGAGGGCGACGATAGACCATTCTTTTGGCTGTTCGAGAATGTGGTGGCTA |
| | TGGGCGTGAGCGATAAGAGGGACATCTCCAGGTTTCTGGAGTCTAACCCCGTGATGATCGAT |
| | GCAAAGGAGGTGTCCGCCGCACACAGAGCCAGGTATTTCTGGGGCAATCTGCCAGGAATGAA |
| | CAGGCCACTGGCAAGCACCGTGAATGACAAGCTGGAGCTGCAGGAGTGCCTGGAGCACGGAA |
| | GGATCGCCAAGTTTTCCAAGGTGCGCACAATCACCACACGGAGCAATTCCATCAAGCAGGGC |
| | AAGGATCAGCACTTCCCCGTGTTCATGAACGAGAAGGAGGACATCCTGTGGTGTACCGAGAT |
| | GGAGAGAGTGTTCGGCTTTCCAGTGCACTACACAGACGTGTCTAACATGAGCAGGCTGGCAA |
| | GGCAGCGGCTGCTGGGCAGATCTTGGAGCGTGCCCGTGATCAGGCACCTGTTCGCCCCTCTG |
| | AAGGAGTATTTTGCCTGCGTGAGCAGCGGCAACTCCAATGCCAACAGCCGGGGCCCCTCTTT |
| | CAGCTCCGGATTGGTGCCTCTGAGCCTGAGGGGCTCCCACATGGCAGCAATCCCCGCCCTGG |
| | ACCCCGAGGCCGAGCCTAGCATGGACGTGATCCTGGTGGGCTCTAGCGAGCTGTCCTCTAGC |
| | GTGTCTCCAGGAACCGGAAGGGATCTGATCGCATACGAGGTGAAGGCCAATCAGCGGAACAT |
| | CGAGGACATCTGTATCTGCTGTGGCAGCCTGCAGGTGCACACACAGCACCCACTGTTCGAGG |
| | GAGGAATCTGCGCACCCTGTAAGGATAAGTTCCTGGACGCCCTGTTTCTGTACGACGATGAC |
| | GGCTACCAGTCCTATTGCTCTATCTGCTGTTCCGGCGAGACCCTGCTGATCTGCGGCAATCC |
| | AGATTGTACAAGGTGCTATTGTTTTGAGTGCGTGGACTCTCTGGTGGGACCAGGCACCAGCG |
| | GAAAGGTGCACGCCATGTCCAACTGGGTGTGCTACCTGTGCCTGCCATCCTCTCGCAGCGGA |
| | CTGCTGCAGCGGAGAAGGAAGTGGAGATCCCAGCTGAAGGCCTTCTATGATAGGGAGTCTGA |
| | GAACCCCCTGGAGATGTTTGAGACCGTGCCAGTGTGGCGCCGGCAGCCCGTGAGGGTGCTGA |
| | GCCTGTTCGAGGATATCAAGAAGGAGCTGACATCCCTGGGCTTTCTGGAGTCCGGCTCTGAC |
| | CCCGGACAGCTGAAGCACGTGGTGGATGTGACCGACACAGTGCGGAAGGATGTGGAGGAGTG |
| | GGGCCCTTTCGACCTGGTGTACGGAGCAACCCCTCCACTGGGACACACATGCGACAGACCCC |
| | CTTCTTGGTACCTGTTCCAGTTTCACCGCCTGCTGCAGTATGCAAGGCCAAAGCCAGGCAGC |
| | CCTAGACCATTCTTTTGGATGTTCGTGGATAATCTGGTGCTGAACAAGGAGGATCTGGACGT |
| | GGCCAGCAGGTTTCTGGAGATGGAGCCAGTGACCATCCCAGACGTGCACGGCGGCTCCCTGC |
| | AGAATGCCGTGCGCGTGTGGTCTAACATCCCTGCCATCAGAAGCAGGCACTGGGCACTGGTG |
| | AGCGAGGAGGAGCTGTCCCTGCTGGCCCAGAATAAGCAGAGCAGCAAGCTGGCCGCCAAGTG |
| | GCCTACAAAGCTGGTGAAGAACTGCTTCCTGCCACTGCGGGAGTACTTCAAGTATTTTTCCA |
| | CCGAGCTGACATCTAGCCTGGGAGGACCCTCCTCTGGCGCCCCACCACCTAGCGGCGGCTCC |
| | CCTGCCGGCTCTCCAACCAGCACAGAGGAGGGCACCAGCGAGTCCGCCACACCAGAGTCTGG |
| | ACCTGGCACCAGCACAGAGCCATCCGAGGGCTCTGCCCCAGGCTCTCCTGCAGGCAGCCCTA |
| | CCTCCACCGAAGAGGGCACCAGCACAGAGCCTTCTGAGGGCAGCGCCCCAGGCACCTCTACA |
| | GAGCCAAGCGAGTCCCGGCCAGGGGAACGGCCCTTCCAGTGTCGGATCTGCATGAGAAACTT |
| | TTCAACACCCTCAAAGCTGGATAGGCATACACGCACCCACACTGGAGAGAAACCCTTTCAGT |
| | GCAGGATATGTATGCGGAATTTTTCCCTGGCTGAGAACCTCCGAAGGCACCTGAGAACACAT |
| | ACCGGGAGTCAGAAGCCTTTCCAATGCCGGATTTGCATGAGGAACTTCTCCCGCCAAGATAA |
| | CCTGGGCCGCCACCTGCGCACACATACAGGCGAGAAGCCATTCCAGTGTAGGATCTGCATGC |
| | GCAATTTTAGCGACGGCGGTAACTTGGGACGCCACCTCAAAACGCATACAGGTGGCGGTGGC |
| | TCCCAGAAGCCCTTTCAGTGCAGGATCTGCATGAGGAATTTTAGTCAGTCCCCACATCTTAA |
| | AAGACACTTGCGGACGCATACTGGAGAGAAGCCCTTTCAGTGTAGGATTTGTATGCGGAACT |
| | TCAGCCAGAGCACAAGCCTGCAACGCCATCTCAAAACTCATTTGCGCGGGTCTGGTGTTGAT |
| | GGCTCCTCTGGTTCTGAAACTCCTGGTACTTCTGAGTCTGCTACTCCCGAATCTGAGAGACC |
| | TTTTCAGTGCCGGATTTGTATGCGCAATTTCTCCCACAAGCAGCACCGCGACGCCCATATTA |
| | GAACACATACTGGAGAAAAGCCCTTCGCTTGCGACATCTGTGGACGGAAGTTCGCCCGCTCC |
| | GCCAACCTGACCCGCCACACTAAAATCCATACCGGCAGCCAAAAGCCATTCCAATGTCGCAT |
| | CTGTATGCGGAACTTTAGCCGCTCCGACAACCTGTCCGAGCATATTCGGACTCACACAGGGG |
| | AGAAACCATTTGCATGTGATATCTGCGGCAGAAAATTCGCTACCTCCGCCAACCTGTCCCGC |
| | CATACCAAGATACACACGGGATCTCAGAAGCCCTTCCAGTGTCGAATCTGCATGCGTAACTT |
| | CAGTATCCGCTCCACCCTGCGCGACCACATCCGCACCCACACCGGCGAGAAGCCTTTTGCCT |
| | GTGACATTTGTGGGAGGAAATTTGCCCGCACCCCGGTGCGCATGGGCCATACCAAGATACAC |
| | CTGCGCCAAAAAGATGCGGCCCGGGGATCTAGCCCCAAGAAGAAGAGAAAGGTGGGAGTCGA |
| | CGGATCCAGCGGCTCCGAGACCCCAGGCACATCTGAGAGCGCCACCCCTGAGTCCACCGGTG |
| | ACTCCGTTGCTTTCGAGGACGTGGCCGTGAACTTCACACTTGAGGAATGGGCCTTGCTCGAC |
| | CCAAGTCAGAAGAATCTGTACAGAGACGTGATGCGGGAGACATTCAGGAATCTCGCCAGTGT |
| | CGGAAAGCAGTGGGAAGACCAGAACATCGAAGATCCTTTCAAGATACCACGGCGCAATATCT |
| | CCCACATTCCTGAGAGGCTGTGTGAATCTAAGGAAGGCGGACAAGGTGAGGAAAGCGCTGAT |
| | TACAAAGATGATGACGATAAAGCCCCCAAGAAGAAAAGGAAGGTCCCAAAGAAAAAAAGAAA |
| | GGTG |
| Fusion protein 2 | MPKKKRKVPKKKRKVYNHDQEFDPPKVYPPVPAEKRKPIRVLSLFDGIATGLLVLKDLGIQV |
| Amino Acid Sequence | DRYIASEVCEDSITVGMVRHQGKIMYVGDVRSVTQKHIQEWGPFDLVIGGSPCNDLSIVNPA |
| CLTA-PCSK9-ZF-Off | RKGLYEGTGRLFFEFYRLLHDARPKEGDDRPFFWLFENVVAMGVSDKRDISRFLESNPVMID |
| 2xNLS-hDNMT3A-W | AKEVSAAHRARYFWGNLPGMNRPLASTVNDKLELQECLEHGRIAKFSKVRTITTRSNSIKQG |
| linker-hDNMT3L- | KDQHFPVFMNEKEDILWCTEMERVFGFPVHYTDVSNMSRLARQRLLGRSWSVPVIRHLFAPL |
| XTEN80-ZFP2244- | KEYFACVSSGNSNANSRGPSFSSGLVPLSLRGSHMAAIPALDPEAEPSMDVILVGSSELSSS |
| XTEN16-ZFP152-NLS- | VSPGTGRDLIAYEVKANQRNIEDICICCGSLQVHTQHPLFEGGICAPCKDKFLDALFLYDDD |
| XTEN16-ZN627KRAB- | GYQSYCSICCSGETLLICGNPDCTRCYCFECVDSLVGPGTSGKVHAMSNWVCYLCLPSSRSG |
| FLAG-2xNLS | LLQRRRKWRSQLKAFYDRESENPLEMFETVPVWRRQPVRVLSLFEDIKKELTSLGFLESGSD |
| SEQ ID NO. 19 | PGQLKHVVDVTDTVRKDVEEWGPFDLVYGATPPLGHTCDRPPSWYLFQFHRLLQYARPKPGS |
| | PRPFFWMFVDNLVLNKEDLDVASRFLEMEPVTIPDVHGGSLQNAVRVWSNIPAIRSRHWALV |
| | SEEELSLLAQNKQSSKLAAKWPTKLVKNCFLPLREYFKYFSTELTSSLGGPSSGAPPPSGGS |
| | PAGSPTSTEEGTSESATPESGPGTSTEPSEGSAPGSPAGSPTSTEEGTSTEPSEGSAPGTST |
| | EPSEERPFQCRICMRNFSHKQHRDAHIRTHTGEKPFACDICGRKFARSANLTRHTKIHTGSQ |
| | KPFQCRICMRNFSRSDNLSEHIRTHTGEKPFACDICGRKFATSANLSRHTKIHTGSQKPFQC |
| | RICMRNFSIRSTLRDHIRTHTGEKPFACDICGRKFARTPVRMGHTKIHLRQKDAARGSGVDG |
| | SSGSETPGTSESATPESSRPGERPFQCRICMRNFSTPSKLDRHTRTHTGEKPFQCRICMRNF |
| | SLAENLRRHLRTHTGSQKPFQCRICMRNFSRQDNLGRHLRTHTGEKPFQCRICMRNFSDGGN |
| | LGRHLKTHTGGGGSQKPFQCRICMRNFSQSPHLKRHLRTHTGEKPFQCRICMRNFSQSTSLQ |
| | RHLKTHLRGSSPKKKRKVGVDGSSGSETPGTSESATPESTGDSVAFEDVAVNFTLEEWALLD |
| | PSQKNLYRDVMRETFRNLASVGKQWEDQNIEDPFKIPRRNISHIPERLCESKEGGQGEESAD |
| | YKDDDDKAPKKKRKVPKKKRKV |
| Fusion protein 2 | ATGCCAAAAAAGAAGAGAAAGGTACCGAAGAAAAAAAGAAAGGTATACAATCACGATCAGGA |
| DNA Sequence | GTTCGACCCCCCTAAGGTGTACCCACCAGTGCCTGCAGAGAAGAGGAAGCCAATCCGGGTGC |
| SEQ ID NO. 20 | TGAGCCTGTTTGATGGCATCGCCACCGGCCTGCTGGTGCTGAAGGATCTGGGCATCCAGGTG |
| | GACCGGTACATCGCCTCCGAGGTGTGCGAGGATTCTATCACCGTGGGCATGGTGCGCCACCA |
| | GGGCAAGATCATGTATGTGGGCGACGTGCGGTCCGTGACACAGAAGCACATCCAGGAGTGGG |
| | GCCCATTCGATCTGGTGATCGGCGGCAGCCCCTGTAATGACCTGTCCATCGTGAACCCTGCA |
| | AGGAAGGGACTGTACGAGGGAACCGGCCGGCTGTTCTTTGAGTTTTATAGACTGCTGCACGA |
| | CGCCAGGCCTAAGGAGGGCGACGATAGACCATTCTTTTGGCTGTTCGAGAATGTGGTGGCTA |
| | TGGGCGTGAGCGATAAGAGGGACATCTCCAGGTTTCTGGAGTCTAACCCCGTGATGATCGAT |
| | GCAAAGGAGGTGTCCGCCGCACACAGAGCCAGGTATTTCTGGGGCAATCTGCCAGGAATGAA |
| | CAGGCCACTGGCAAGCACCGTGAATGACAAGCTGGAGCTGCAGGAGTGCCTGGAGCACGGAA |
| | GGATCGCCAAGTTTTCCAAGGTGCGCACAATCACCACACGGAGCAATTCCATCAAGCAGGGC |
| | AAGGATCAGCACTTCCCCGTGTTCATGAACGAGAAGGAGGACATCCTGTGGTGTACCGAGAT |
| | GGAGAGAGTGTTCGGCTTTCCAGTGCACTACACAGACGTGTCTAACATGAGCAGGCTGGCAA |
| | GGCAGCGGCTGCTGGGCAGATCTTGGAGCGTGCCCGTGATCAGGCACCTGTTCGCCCCTCTG |
| | AAGGAGTATTTTGCCTGCGTGAGCAGCGGCAACTCCAATGCCAACAGCCGGGGCCCCTCTTT |
| | CAGCTCCGGATTGGTGCCTCTGAGCCTGAGGGGCTCCCACATGGCAGCAATCCCCGCCCTGG |
| | ACCCCGAGGCCGAGCCTAGCATGGACGTGATCCTGGTGGGCTCTAGCGAGCTGTCCTCTAGC |
| | GTGTCTCCAGGAACCGGAAGGGATCTGATCGCATACGAGGTGAAGGCCAATCAGCGGAACAT |
| | CGAGGACATCTGTATCTGCTGTGGCAGCCTGCAGGTGCACACACAGCACCCACTGTTCGAGG |
| | GAGGAATCTGCGCACCCTGTAAGGATAAGTTCCTGGACGCCCTGTTTCTGTACGACGATGAC |
| | GGCTACCAGTCCTATTGCTCTATCTGCTGTTCCGGCGAGACCCTGCTGATCTGCGGCAATCC |
| | AGATTGTACAAGGTGCTATTGTTTTGAGTGCGTGGACTCTCTGGTGGGACCAGGCACCAGCG |
| | GAAAGGTGCACGCCATGTCCAACTGGGTGTGCTACCTGTGCCTGCCATCCTCTCGCAGCGGA |
| | CTGCTGCAGCGGAGAAGGAAGTGGAGATCCCAGCTGAAGGCCTTCTATGATAGGGAGTCTGA |
| | GAACCCCCTGGAGATGTTTGAGACCGTGCCAGTGTGGCGCCGGCAGCCCGTGAGGGTGCTGA |
| | GCCTGTTCGAGGATATCAAGAAGGAGCTGACATCCCTGGGCTTTCTGGAGTCCGGCTCTGAC |
| | CCCGGACAGCTGAAGCACGTGGTGGATGTGACCGACACAGTGCGGAAGGATGTGGAGGAGTG |
| | GGGCCCTTTCGACCTGGTGTACGGAGCAACCCCTCCACTGGGACACACATGCGACAGACCCC |
| | CTTCTTGGTACCTGTTCCAGTTTCACCGCCTGCTGCAGTATGCAAGGCCAAAGCCAGGCAGC |
| | CCTAGACCATTCTTTTGGATGTTCGTGGATAATCTGGTGCTGAACAAGGAGGATCTGGACGT |
| | GGCCAGCAGGTTTCTGGAGATGGAGCCAGTGACCATCCCAGACGTGCACGGCGGCTCCCTGC |
| | AGAATGCCGTGCGCGTGTGGTCTAACATCCCTGCCATCAGAAGCAGGCACTGGGCACTGGTG |
| | AGCGAGGAGGAGCTGTCCCTGCTGGCCCAGAATAAGCAGAGCAGCAAGCTGGCCGCCAAGTG |
| | GCCTACAAAGCTGGTGAAGAACTGCTTCCTGCCACTGCGGGAGTACTTCAAGTATTTTTCCA |
| | CCGAGCTGACATCTAGCCTGGGAGGACCCTCCTCTGGCGCCCCACCACCTAGCGGCGGCTCC |
| | CCTGCCGGCTCTCCAACCAGCACAGAGGAGGGCACCAGCGAGTCCGCCACACCAGAGTCTGG |
| | ACCTGGCACCAGCACAGAGCCATCCGAGGGCTCTGCCCCAGGCTCTCCTGCAGGCAGCCCTA |
| | CCTCCACCGAAGAGGGCACCAGCACAGAGCCTTCTGAGGGCAGCGCCCCAGGCACCTCTACA |
| | GAGCCAAGCGAGGAGAGACCTTTTCAGTGCCGGATTTGTATGCGCAATTTCTCCCACAAGCA |
| | GCACCGCGACGCCCATATTAGAACACATACTGGAGAAAAGCCCTTCGCTTGCGACATCTGTG |
| | GACGGAAGTTCGCCCGCTCCGCCAACCTGACCCGCCACACTAAAATCCATACCGGCAGCCAA |
| | AAGCCATTCCAATGTCGCATCTGTATGCGGAACTTTAGCCGCTCCGACAACCTGTCCGAGCA |
| | TATTCGGACTCACACAGGGGAGAAACCATTTGCATGTGATATCTGCGGCAGAAAATTCGCTA |
| | CCTCCGCCAACCTGTCCCGCCATACCAAGATACACACGGGATCTCAGAAGCCCTTCCAGTGT |
| | CGAATCTGCATGCGTAACTTCAGTATCCGCTCCACCCTGCGCGACCACATCCGCACCCACAC |
| | CGGCGAGAAGCCTTTTGCCTGTGACATTTGTGGGAGGAAATTTGCCCGCACCCCGGTGCGCA |
| | TGGGCCATACCAAGATACACCTGCGCCAAAAAGATGCGGCCCGGGGATCTGGTGTTGATGGC |
| | TCCTCTGGTTCTGAAACTCCTGGTACTTCTGAGTCTGCTACTCCCGAATCTTCCCGGCCAGG |
| | GGAACGGCCCTTCCAGTGTCGGATCTGCATGAGAAACTTTTCAACACCCTCAAAGCTGGATA |
| | GGCATACACGCACCCACACTGGAGAGAAACCCTTTCAGTGCAGGATATGTATGCGGAATTTT |
| | TCCCTGGCTGAGAACCTCCGAAGGCACCTGAGAACACATACCGGGAGTCAGAAGCCTTTCCA |
| | ATGCCGGATTTGCATGAGGAACTTCTCCCGCCAAGATAACCTGGGCCGCCACCTGCGCACAC |
| | ATACAGGCGAGAAGCCATTCCAGTGTAGGATCTGCATGCGCAATTTTAGCGACGGCGGTAAC |
| | TTGGGACGCCACCTCAAAACGCATACAGGTGGCGGTGGCTCCCAGAAGCCCTTTCAGTGCAG |
| | GATCTGCATGAGGAATTTTAGTCAGTCCCCACATCTTAAAAGACACTTGCGGACGCATACTG |
| | GAGAGAAGCCCTTTCAGTGTAGGATTTGTATGCGGAACTTCAGCCAGAGCACAAGCCTGCAA |
| | CGCCATCTCAAAACTCATTTGCGCGGGTCTAGCCCCAAGAAGAAGAGAAAGGTGGGAGTCGA |
| | CGGATCCAGCGGCTCCGAGACCCCAGGCACATCTGAGAGCGCCACCCCTGAGTCCACCGGTG |
| | ACTCCGTTGCTTTCGAGGACGTGGCCGTGAACTTCACACTTGAGGAATGGGCCTTGCTCGAC |
| | CCAAGTCAGAAGAATCTGTACAGAGACGTGATGCGGGAGACATTCAGGAATCTCGCCAGTGT |
| | CGGAAAGCAGTGGGAAGACCAGAACATCGAAGATCCTTTCAAGATACCACGGCGCAATATCT |
| | CCCACATTCCTGAGAGGCTGTGTGAATCTAAGGAAGGCGGACAAGGTGAGGAAAGCGCTGAT |
| | TACAAAGATGATGACGATAAAGCCCCCAAGAAGAAAAGGAAGGTCCCAAAGAAAAAAAGAAA |
| | GGTGTGA |
| Fusion protein 3 | MPKKKRKVPKKKRKVYDSVAFEDVAVNFTLEEWALLDPSQKNLYRDVMRETFRNLASVGKQW |
| Amino Acid Sequence | EDQNIEDPFKIPRRNISHIPERLCESKEGGQGEESGVDGSSGSETPGTSESATPESERPFQC |
| Palindromic | RICMRNFSHKQHRDAHIRTHTGEKPFACDICGRKFARSANLTRHTKIHTGSQKPFQCRICMR |
| CLTA/PCSK9 ZF-OFF | NFSRSDNLSEHIRTHTGEKPFACDICGRKFATSANLSRHTKIHTGSQKPFQCRICMRNFSIR |
| 2xNLS-ZN627KRAB- | STLRDHIRTHTGEKPFACDICGRKFARTPVRMGHTKIHLRQKDAARGSGGPSSGAPPPSGGS |
| XTEN16-ZFP2244- | PAGSPTSTEEGTSESATPESGPGTSTEPSEGSAPGSPAGSPTSTEEGTSTEPSEGSAPGTST |
| XTEN80-hDNMT3A-W | EPSENHDQEFDPPKVYPPVPAEKRKPIRVLSLFDGIATGLLVLKDLGIQVDRYIASEVCEDS |
| linker-hDNMT3L- | ITVGMVRHQGKIMYVGDVRSVTQKHIQEWGPFDLVIGGSPCNDLSIVNPARKGLYEGTGRLF |
| XTEN80-NLS-ZFP152- | FEFYRLLHDARPKEGDDRPFFWLFENVVAMGVSDKRDISRFLESNPVMIDAKEVSAAHRARY |
| XTEN16-ZN627KRAB- | FWGNLPGMNRPLASTVNDKLELQECLEHGRIAKFSKVRTITTRSNSIKQGKDQHFPVFMNEK |
| FLAG-2xNLS | EDILWCTEMERVFGFPVHYTDVSNMSRLARQRLLGRSWSVPVIRHLFAPLKEYFACVSSGNS |
| SEQ ID NO. 21 | NANSRGPSFSSGLVPLSLRGSHMAAIPALDPEAEPSMDVILVGSSELSSSVSPGTGRDLIAY |
| | EVKANQRNIEDICICCGSLQVHTQHPLFEGGICAPCKDKFLDALFLYDDDGYQSYCSICCSG |
| | ETLLICGNPDCTRCYCFECVDSLVGPGTSGKVHAMSNWVCYLCLPSSRSGLLQRRRKWRSQL |
| | KAFYDRESENPLEMFETVPVWRRQPVRVLSLFEDIKKELTSLGFLESGSDPGQLKHVVDVTD |
| | TVRKDVEEWGPFDLVYGATPPLGHTCDRPPSWYLFQFHRLLQYARPKPGSPRPFFWMFVDNL |
| | VLNKEDLDVASRFLEMEPVTIPDVHGGSLQNAVRVWSNIPAIRSRHWALVSEEELSLLAQNK |
| | QSSKLAAKWPTKLVKNCFLPLREYFKYFSTELTSSLGGPSSGAPPPSGGSPAGSPTSTEEGT |
| | SESATPESGPGTSTEPSEGSAPGSPAGSPTSTEEGTSTEPSEGSAPGTSTEPSEPKKKRKVS |
| | RPGERPFQCRICMRNFSTPSKLDRHTRTHTGEKPFQCRICMRNFSLAENLRRHLRTHTGSQK |
| | PFQCRICMRNFSRQDNLGRHLRTHTGEKPFQCRICMRNFSDGGNLGRHLKTHTGGGGSQKPF |
| | QCRICMRNFSQSPHLKRHLRTHTGEKPFQCRICMRNFSQSTSLQRHLKTHLRGSSGVDGSSG |
| | SETPGTSESATPESTGDSVAFEDVAVNFTLEEWALLDPSQKNLYRDVMRETFRNLASVGKQW |
| | EDQNIEDPFKIPRRNISHIPERLCESKEGGQGEESADYKDDDDKAPKKKRKVPKKKRKV |
| Fusion protein 3 | ATGCCAAAAAAGAAGAGAAAGGTACCGAAGAAAAAAAGAAAGGTATACGACTCCGTTGCTTT |
| DNA Sequence | CGAGGACGTGGCCGTGAACTTCACACTTGAGGAATGGGCCTTGCTCGACCCAAGTCAGAAGA |
| SEQ ID NO. 22 | ATCTGTACAGAGACGTGATGCGGGAGACATTCAGGAATCTCGCCAGTGTCGGAAAGCAGTGG |
| | GAAGACCAGAACATCGAAGATCCTTTCAAGATACCACGGCGCAATATCTCCCACATTCCTGA |
| | GAGGCTGTGTGAATCTAAGGAAGGCGGACAAGGTGAGGAAAGCGGAGTCGACGGATCCAGCG |
| | GCTCCGAGACCCCAGGCACATCTGAGAGCGCCACCCCTGAGTCCGAGAGACCTTTTCAGTGC |
| | CGGATTTGTATGCGCAATTTCTCCCACAAGCAGCACCGCGACGCCCATATTAGAACACATAC |
| | TGGAGAAAAGCCCTTCGCTTGCGACATCTGTGGACGGAAGTTCGCCCGCTCCGCCAACCTGA |
| | CCCGCCACACTAAAATCCATACCGGCAGCCAAAAGCCATTCCAATGTCGCATCTGTATGCGG |
| | AACTTTAGCCGCTCCGACAACCTGTCCGAGCATATTCGGACTCACACAGGGGAGAAACCATT |
| | TGCATGTGATATCTGCGGCAGAAAATTCGCTACCTCCGCCAACCTGTCCCGCCATACCAAGA |
| | TACACACGGGATCTCAGAAGCCCTTCCAGTGTCGAATCTGCATGCGTAACTTCAGTATCCGC |
| | TCCACCCTGCGCGACCACATCCGCACCCACACCGGCGAGAAGCCTTTTGCCTGTGACATTTG |
| | TGGGAGGAAATTTGCCCGCACCCCGGTGCGCATGGGCCATACCAAGATACACCTGCGCCAAA |
| | AAGATGCGGCCCGGGGATCTGGAGGACCCTCCTCTGGCGCCCCACCACCTAGCGGCGGCTCC |
| | CCTGCCGGCTCTCCAACCAGCACAGAGGAGGGCACCAGCGAGTCCGCCACACCAGAGTCTGG |
| | ACCTGGCACCAGCACAGAGCCATCCGAGGGCTCTGCCCCAGGCTCTCCTGCAGGCAGCCCTA |
| | CCTCCACCGAAGAGGGCACCAGCACAGAGCCTTCTGAGGGCAGCGCCCCAGGCACCTCTACA |
| | GAGCCAAGCGAGAATCACGATCAGGAGTTCGACCCCCCTAAGGTGTACCCACCAGTGCCTGC |
| | AGAGAAGAGGAAGCCAATCCGGGTGCTGAGCCTGTTTGATGGCATCGCCACCGGCCTGCTGG |
| | TGCTGAAGGATCTGGGCATCCAGGTGGACCGGTACATCGCCTCCGAGGTGTGCGAGGATTCT |
| | ATCACCGTGGGCATGGTGCGCCACCAGGGCAAGATCATGTATGTGGGCGACGTGCGGTCCGT |
| | GACACAGAAGCACATCCAGGAGTGGGGCCCATTCGATCTGGTGATCGGCGGCAGCCCCTGTA |
| | ATGACCTGTCCATCGTGAACCCTGCAAGGAAGGGACTGTACGAGGGAACCGGCCGGCTGTTC |
| | TTTGAGTTTTATAGACTGCTGCACGACGCCAGGCCTAAGGAGGGCGACGATAGACCATTCTT |
| | TTGGCTGTTCGAGAATGTGGTGGCTATGGGCGTGAGCGATAAGAGGGACATCTCCAGGTTTC |
| | TGGAGTCTAACCCCGTGATGATCGATGCAAAGGAGGTGTCCGCCGCACACAGAGCCAGGTAT |
| | TTCTGGGGCAATCTGCCAGGAATGAACAGGCCACTGGCAAGCACCGTGAATGACAAGCTGGA |
| | GCTGCAGGAGTGCCTGGAGCACGGAAGGATCGCCAAGTTTTCCAAGGTGCGCACAATCACCA |
| | CACGGAGCAATTCCATCAAGCAGGGCAAGGATCAGCACTTCCCCGTGTTCATGAACGAGAAG |
| | GAGGACATCCTGTGGTGTACCGAGATGGAGAGAGTGTTCGGCTTTCCAGTGCACTACACAGA |
| | CGTGTCTAACATGAGCAGGCTGGCAAGGCAGCGGCTGCTGGGCAGATCTTGGAGCGTGCCCG |
| | TGATCAGGCACCTGTTCGCCCCTCTGAAGGAGTATTTTGCCTGCGTGAGCAGCGGCAACTCC |
| | AATGCCAACAGCCGGGGCCCCTCTTTCAGCTCCGGATTGGTGCCTCTGAGCCTGAGGGGCTC |
| | CCACATGGCAGCAATCCCCGCCCTGGACCCCGAGGCCGAGCCTAGCATGGACGTGATCCTGG |
| | TGGGCTCTAGCGAGCTGTCCTCTAGCGTGTCTCCAGGAACCGGAAGGGATCTGATCGCATAC |
| | GAGGTGAAGGCCAATCAGCGGAACATCGAGGACATCTGTATCTGCTGTGGCAGCCTGCAGGT |
| | GCACACACAGCACCCACTGTTCGAGGGAGGAATCTGCGCACCCTGTAAGGATAAGTTCCTGG |
| | ACGCCCTGTTTCTGTACGACGATGACGGCTACCAGTCCTATTGCTCTATCTGCTGTTCCGGC |
| | GAGACCCTGCTGATCTGCGGCAATCCAGATTGTACAAGGTGCTATTGTTTTGAGTGCGTGGA |
| | CTCTCTGGTGGGACCAGGCACCAGCGGAAAGGTGCACGCCATGTCCAACTGGGTGTGCTACC |
| | TGTGCCTGCCATCCTCTCGCAGCGGACTGCTGCAGCGGAGAAGGAAGTGGAGATCCCAGCTG |
| | AAGGCCTTCTATGATAGGGAGTCTGAGAACCCCCTGGAGATGTTTGAGACCGTGCCAGTGTG |
| | GCGCCGGCAGCCCGTGAGGGTGCTGAGCCTGTTCGAGGATATCAAGAAGGAGCTGACATCCC |
| | TGGGCTTTCTGGAGTCCGGCTCTGACCCCGGACAGCTGAAGCACGTGGTGGATGTGACCGAC |
| | ACAGTGCGGAAGGATGTGGAGGAGTGGGGCCCTTTCGACCTGGTGTACGGAGCAACCCCTCC |
| | ACTGGGACACACATGCGACAGACCCCCTTCTTGGTACCTGTTCCAGTTTCACCGCCTGCTGC |
| | AGTATGCAAGGCCAAAGCCAGGCAGCCCTAGACCATTCTTTTGGATGTTCGTGGATAATCTG |
| | GTGCTGAACAAGGAGGATCTGGACGTGGCCAGCAGGTTTCTGGAGATGGAGCCAGTGACCAT |
| | CCCAGACGTGCACGGCGGCTCCCTGCAGAATGCCGTGCGCGTGTGGTCTAACATCCCTGCCA |
| | TCAGAAGCAGGCACTGGGCACTGGTGAGCGAGGAGGAGCTGTCCCTGCTGGCCCAGAATAAG |
| | CAGAGCAGCAAGCTGGCCGCCAAGTGGCCTACAAAGCTGGTGAAGAACTGCTTCCTGCCACT |
| | GCGGGAGTACTTCAAGTATTTTTCCACCGAGCTGACATCTAGCCTGGGAGGACCCTCCTCTG |
| | GCGCCCCACCACCTAGCGGCGGCTCCCCTGCCGGCTCTCCAACCAGCACAGAGGAGGGCACC |
| | AGCGAGTCCGCCACACCAGAGTCTGGACCTGGCACCAGCACAGAGCCATCCGAGGGCTCTGC |
| | CCCAGGCTCTCCTGCAGGCAGCCCTACCTCCACCGAAGAGGGCACCAGCACAGAGCCTTCTG |
| | AGGGCAGCGCCCCAGGCACCTCTACAGAGCCAAGCGAGCCCAAGAAGAAGAGGAAGGTGTCC |
| | CGGCCAGGGGAACGGCCCTTCCAGTGTCGGATCTGCATGAGAAACTTTTCAACACCCTCAAA |
| | GCTGGATAGGCATACACGCACCCACACTGGAGAGAAACCCTTTCAGTGCAGGATATGTATGC |
| | GGAATTTTTCCCTGGCTGAGAACCTCCGAAGGCACCTGAGAACACATACCGGGAGTCAGAAG |
| | CCTTTCCAATGCCGGATTTGCATGAGGAACTTCTCCCGCCAAGATAACCTGGGCCGCCACCT |
| | GCGCACACATACAGGCGAGAAGCCATTCCAGTGTAGGATCTGCATGCGCAATTTTAGCGACG |
| | GCGGTAACTTGGGACGCCACCTCAAAACGCATACAGGTGGCGGTGGCTCCCAGAAGCCCTTT |
| | CAGTGCAGGATCTGCATGAGGAATTTTAGTCAGTCCCCACATCTTAAAAGACACTTGCGGAC |
| | GCATACTGGAGAGAAGCCCTTTCAGTGTAGGATTTGTATGCGGAACTTCAGCCAGAGCACAA |
| | GCCTGCAACGCCATCTCAAAACTCATTTGCGCGGGTCTAGCGGAGTCGACGGATCCAGCGGC |
| | TCCGAGACCCCAGGCACATCTGAGAGCGCCACCCCTGAGTCCACCGGTGACTCCGTTGCTTT |
| | CGAGGACGTGGCCGTGAACTTCACACTTGAGGAATGGGCCTTGCTCGACCCAAGTCAGAAGA |
| | ATCTGTACAGAGACGTGATGCGGGAGACATTCAGGAATCTCGCCAGTGTCGGAAAGCAGTGG |
| | GAAGACCAGAACATCGAAGATCCTTTCAAGATACCACGGCGCAATATCTCCCACATTCCTGA |
| | GAGGCTGTGTGAATCTAAGGAAGGCGGACAAGGTGAGGAAAGCGCTGATTACAAAGATGATG |
| | ACGATAAAGCCCCCAAGAAGAAAAGGAAGGTCCCAAAGAAAAAAAGAAAGGTG |

### Example 2: Testing Dual Targeting Zinc Hands Fusion Proteins

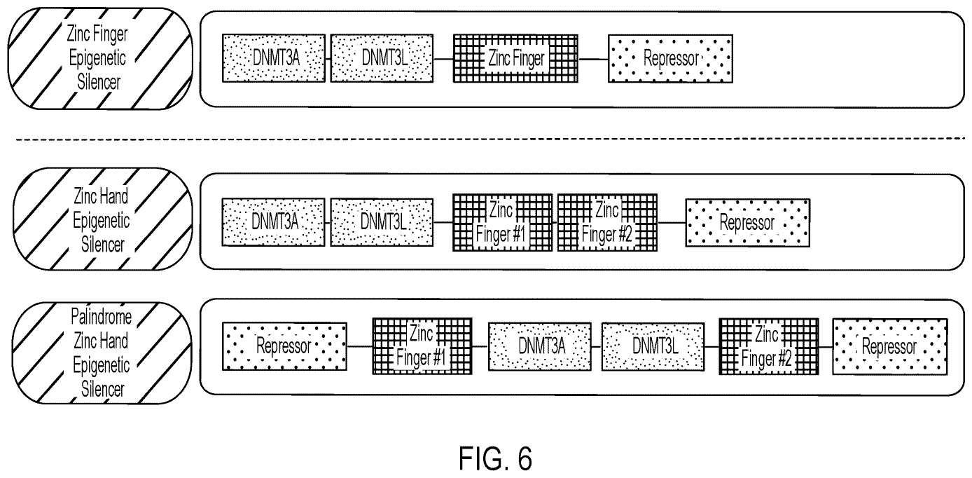

To validate the approach of incorporating multiple zinc finger DNA-binding domains to a single epigenetic editor, a dual targeting system was tested. Zinc Hand and Palindrome epigenetic silencers (see FIG. 6 for schematics) were tested versus a zinc finger silencer.

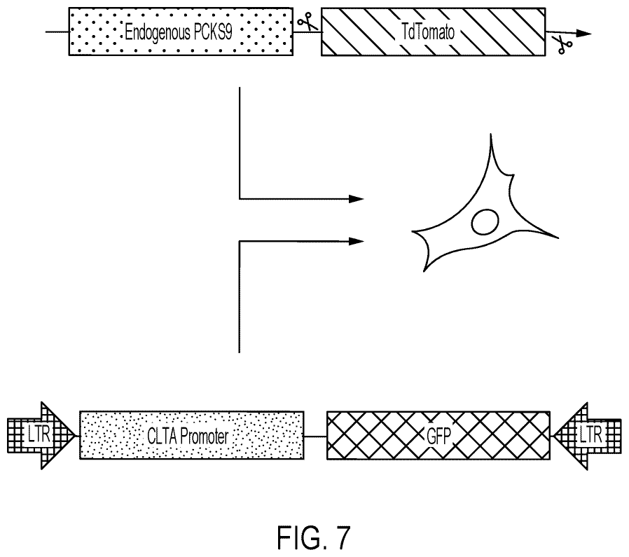

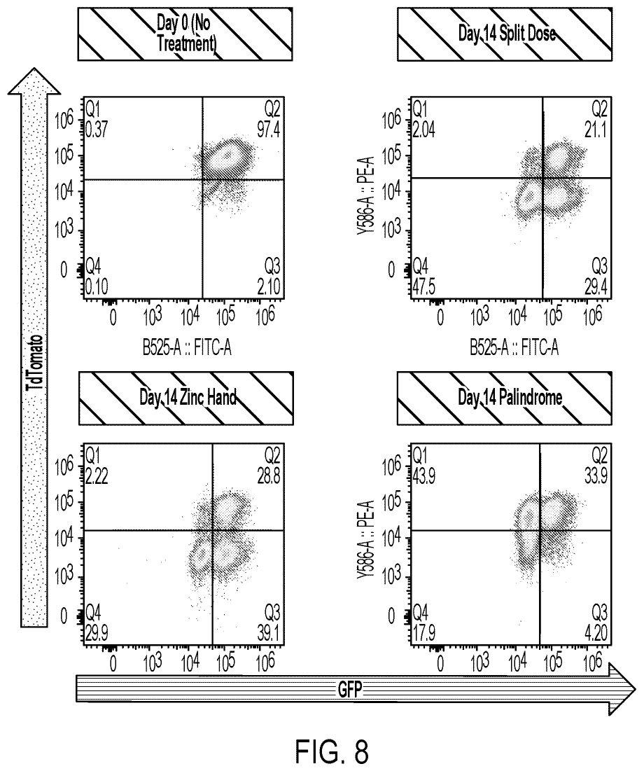

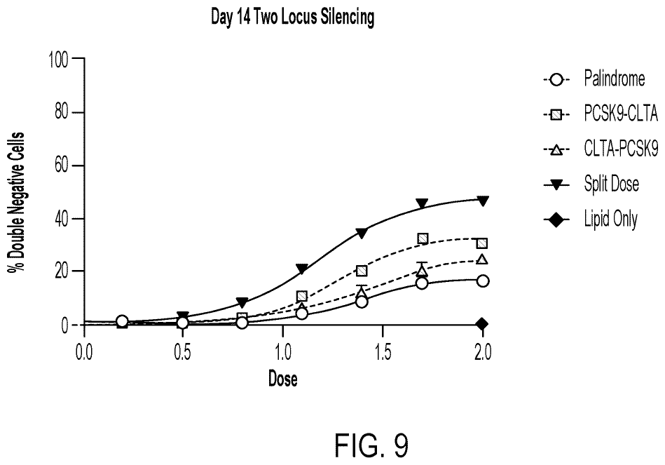

HeLa cells with two fluorescent reporters were made by TdTomato HDR knock-in at the 3′ end of the endogenous PCSK9 locus and lentiviral insertion of a CLTA promoter driven GFP (see FIG. 7 for schematic). Zinc Hand and Palindrome epigenetic silencers targeting the PCSK9 and CLTA genes were transfected into the cell line. As a control, some cells were transfected with two individual single zinc finger epigenetic silencers (Split Dose)-one targeting PCSK9 (amino acid SEQ ID NO: 13, nucleic acid SEQ ID NO: 14) and the other targeting CLTA (amino acid SEQ ID NO: 15, nucleic acid SEQ ID NO: 16). Cells were analyzed via flow cytometry at Day 14. Results for 50 ng total mRNA are shown in FIG. 8. Dose response was also tested using the same split dose and Palindrome constructs and the same and an additional construct for Zinc Hand (an additional orientation, amino acid SEQ ID NO: 19, nucleic acid SEQ ID NO: 20). The percent population of double negative cells was determined at day 14 via flow cytometry. Both zinc hand constructs and the palindrome produce double-silenced cell populations (FIG. 9).

### Example 3: Testing Dose Response of Zinc Hands Fusion Proteins Engineered Against a Single Gene

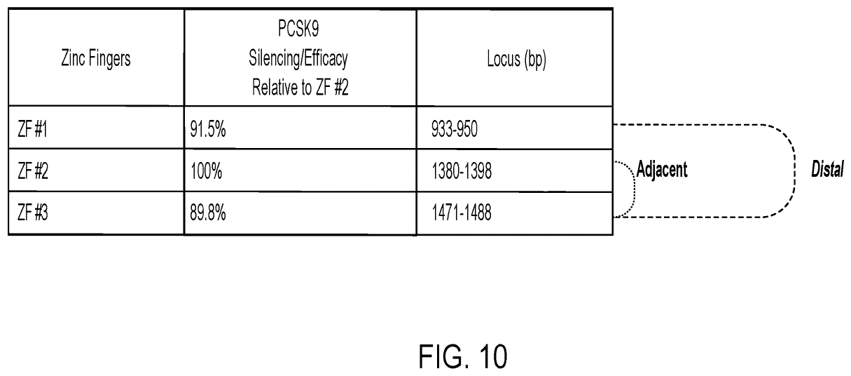

The zinc hands were designed using the three zinc fingers shown in FIG. 10. Sequences are in Table 5.

TABLE 5
Zinc fingers

| | |
|---|---|
| ZF #1 | SRPGERPFQCRICMRNFSRTDTLARHLRTHTGEKP |
| amino | FQCRICMRNFSRKTALNRHLKTHTGSQKPFQCRI |
| acid | CMRNFSRNESLKVHLRTHTGEKPFQCRICMRNFS |
| | VKNTLTRHLKTHTGSQKPFQCRICMRNFSRREHL |
| | VRHLRTHTGEKPFQCRICMRNFSISHNLARHLKT |
| | HLRGS (SEQ ID NO: 707) |
| ZF #1 | TCCCGGCCAGGGGAACGGCCCTTCCAGTGTCG |
| nucleic | GATCTGCATGAGAAACTTTTCAAGAACAGACA |
| acid | CTCTGGCCCGACACCTGAGGACCCACACTGGA |
| | GAGAAACCCTTTCAGTGCAGGATATGTATGCG |
| | GAATTTTTCCAGAAAGACCGCACTTAATCGACA |
| | TCTCAAGACACATACCGGGAGTCAGAAGCCTT |
| | TCCAATGCCGGATTTGCATGAGGAACTTCTCCC |
| | GGAACGAATCTCTGAAGGTGCACCTGCGCACA |
| | CATACAGGCGAGAAGCCATTCCAGTGTAGGAT |
| | CTGCATGCGCAATTTTAGCGTGAAGAATACACT |
| | GACGAGACATCTGAAGACGCATACAGGTTCCC |
| | AGAAGCCCTTTCAGTGCAGGATCTGCATGAGG |
| | AATTTTAGTAGAAGGGAGCATCTCGTTAGACA |
| | CCTCCGGACGCATACTGGAGAGAAGCCCTTTC |
| | AGTGTAGGATTTGTATGCGGAACTTCAGCATAA |
| | GCCACAATCTGGCAAGGCACCTTAAAACTCATT |
| | TGCGCGGGTCT (SEQ ID NO: 708) |
| ZF #1 | GATGGGGCTCTGGTGGCG (SEQ ID NO: 709) |
| target | |
| sequence | |
| ZF #2 | SRPGERPFQCRICMRNFSTPSKLDRHTRTHTGEKP |
| amino | FQCRICMRNFSLAENLRRHLRTHTGSQKPFQCRIC |
| acid | MRNFSRQDNLGRHLRTHTGEKPFQCRICMRNFSD |
| | GGNLGRHLKTHTGGGGSQKPFQCRICMRNFSQSP |
| | HLKRHLRTHTGEKPFQCRICMRNFSQSTSLQRHL |
| | KTHLRGS (SEQ ID NO: 1) |
| ZF #2 | TCCCGGCCAGGGGAACGGCCCTTCCAGTGTCG |
| nucleic | GATCTGCATGAGAAACTTTTCAACACCCTCAAA |
| acid | GCTGGATAGGCATACACGCACCCACACTGGAG |
| | AGAAACCCTTTCAGTGCAGGATATGTATGCGG |
| | AATTTTTCCCTGGCTGAGAACCTCCGAAGGCAC |
| | CTGAGAACACATACCGGGAGTCAGAAGCCTTT |
| | CCAATGCCGGATTTGCATGAGGAACTTCTCCCG |
| | CCAAGATAACCTGGGCCGCCACCTGCGCACAC |
| | ATACAGGCGAGAAGCCATTCCAGTGTAGGATC |
| | TGCATGCGCAATTTTAGCGACGGCGGTAACTTG |
| | GGACGCCACCTCAAAACGCATACAGGTGGCGG |
| | TGGCTCCCAGAAGCCCTTTCAGTGCAGGATCTG |
| | CATGAGGAATTTTAGTCAGTCCCCACATCTTAA |
| | AAGACACTTGCGGACGCATACTGGAGAGAAGC |
| | CCTTTCAGTGTAGGATTTGTATGCGGAACTTCA |
| | GCCAGAGCACAAGCCTGCAACGCCATCTCAAA |
| | ACTCATTTGCGCGGGTCT (SEQ ID NO: 2) |
| ZF #2 | GCAGGAGGACGAGGACGGC (SEQ ID NO: 5) |
| target | |
| sequence | |
| ZF #3 | SRPGERPFQCRICMRNFSRRQHLTLHTRTHTGEKP |
| amino | FQCRICMRNFSDNSHLQRHLRTHTGSQKPFQCRI |
| acid | CMRNFSLPHHLQRHLRTHTGEKPFQCRICMRNFS |
| | QSNNLTRHLKTHTGSQKPFQCRICMRNFSRNFILQ |
| | RHTRTHTGEKPFQCRICMRNFSRKDDLKRHLRTH |
| | LRGS (SEQ ID NO: 710) |
| ZF #3 | TCCCGGCCAGGGGAACGGCCCTTCCAGTGTCG |
| nucleic | GATCTGCATGAGAAACTTTTCACGCAGACAGC |
| acid | ACCTGACACTCCACACTCGAACCCACACTGGA |
| | GAGAAACCCTTTCAGTGCAGGATATGTATGCG |
| | GAATTTTTCCGACAACAGCCACCTGCAAAGGC |
| | ATCTCCGCACACATACCGGGAGTCAGAAGCCT |
| | TTCCAATGCCGGATTTGCATGAGGAACTTCTCC |
| | CTTCCCCACCATCTGCAGCGCCACCTGCGGACA |
| | CATACAGGCGAGAAGCCATTCCAGTGTAGGAT |
| | CTGCATGCGCAATTTTAGCCAGTCTAATAACCT |
| | TACTCGCCACCTCAAGACGCATACAGGTTCCCA |
| | GAAGCCCTTTCAGTGCAGGATCTGCATGAGGA |
| | ATTTTAGTAGGAACTTCATACTGCAACGGCACA |
| | CAAGAACGCATACTGGAGAGAAGCCCTTTCAG |
| | TGTAGGATTTGTATGCGGAACTTCAGCAGGAA |
| | GGACGATCTGAAAAGACATCTGAGAACTCATT |
| | TGCGCGGGTCT (SEQ ID NO: 711) |
| ZF #3 | GCGGTGGAAGGTGGCTGT (SEQ ID NO: 712) |
| target | |
| sequence | |

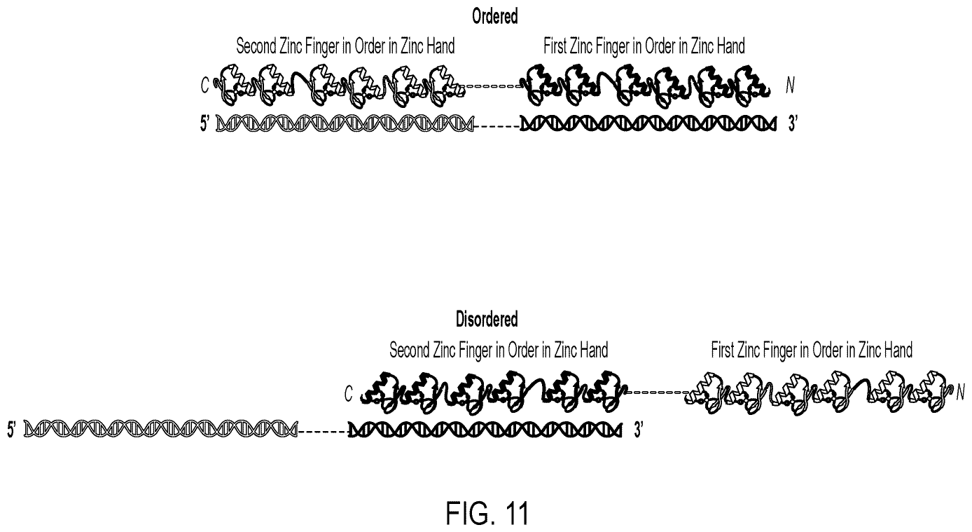

Epigenetic silencers designed with these zinc fingers are known to perform with similar efficacy (data not shown). Each of these zinc fingers target different sites within PCSK9 as shown. The constructs are classified based on distance between the target site of each constitutive zinc finger (distal, adjacent, or redundant). Zinc Hands were designed incorporating ZF #2+3 (adjacent), ZF #1+3 (distal), and or ZF #2+2 (redundant). The constructs are further characterized by their spatial orientation: zinc hands binding such that the finger orientation N to C aligns with DNA sequence orientation 3′ to 5′ are labeled as “ordered” and those that have a disrupted orientation are labeled as “disordered”. Ordered zinc hands are arranged with a spatial advantage expected to yield better silencing ability over disordered zinc hands. Schematics of ordered and disordered orientations are shown in FIG. 11. Sequences of the constructs are in Table 6. The target sequences for each component are the same as listed in Table 5.

TABLE 6
Fusion protein constructs for dose response

| | |
|---|---|
| ZF #1 + ZF #3 | MPKKKRKVPKKKRKVYNHDQEFDPPKVYPPVPA |
| (Distal, Disordered) | EKRKPIRVLSLFDGIATGLLVLKDLGIQVDRYIAS |
| amino acid | EVCEDSITVGMVRHQGKIMYVGDVRSVTQKHIQ |
| | EWGPFDLVIGGSPCNDLSIVNPARKGLYEGTGRL |
| | FFEFYRLLHDARPKEGDDRPFFWLFENVVAMGV |
| | SDKRDISRFLESNPVMIDAKEVSAAHRARYFWGN |
| | LPGMNRPLASTVNDKLELQECLEHGRIAKFSKVR |
| | TITTRSNSIKQGKDQHFPVFMNEKEDILWCTEME |
| | RVFGFPVHYTDVSNMSRLARQRLLGRSWSVPVIR |
| | HLFAPLKEYFACVSSGNSNANSRGPSFSSGLVPLS |
| | LRGSHMAAIPALDPEAEPSMDVILVGSSELSSSVS |
| | PGTGRDLIAYEVKANQRNIEDICICCGSLQVHTQH |
| | PLFEGGICAPCKDKFLDALFLYDDDGYQSYCSICC |
| | SGETLLICGNPDCTRCYCFECVDSLVGPGTSGKV |
| | HAMSNWVCYLCLPSSRSGLLQRRRKWRSQLKAF |
| | YDRESENPLEMFETVPVWRRQPVRVLSLFEDIKK |
| | ELTSLGFLESGSDPGQLKHVVDVTDTVRKDVEE |
| | WGPFDLVYGATPPLGHTCDRPPSWYLFQFHRLL |
| | QYARPKPGSPRPFFWMFVDNLVLNKEDLDVASR |
| | FLEMEPVTIPDVHGGSLQNAVRVWSNIPAIRSRH |
| | WALVSEEELSLLAQNKQSSKLAAKWPTKLVKNC |
| | FLPLREYFKYFSTELTSSLGGPSSGAPPPSGGSPAG |
| | SPTSTEEGTSESATPESGPGTSTEPSEGSAPGSPAG |
| | SPTSTEEGTSTEPSEGSAPGTSTEPSESRPGERPFQ |
| | CRICMRNFSRTDTLARHLRTHTGEKPFQCRICMR |
| | NFSRKTALNRHLKTHTGSQKPFQCRICMRNFSRN |
| | ESLKVHLRTHTGEKPFQCRICMRNFSVKNTLTRH |
| | LKTHTGSQKPFQCRICMRNFSRREHLVRHLRTHT |
| | GEKPFQCRICMRNFSISHNLARHLKTHLRGSGVD |
| | GSSGSETPGTSESATPESSRPGERPFQCRICMRNFS |
| | RRQHLTLHTRTHTGEKPFQCRICMRNFSDNSHLQ |
| | RHLRTHTGSQKPFQCRICMRNFSLPHHLQRHLRT |
| | HTGEKPFQCRICMRNFSQSNNLTRHLKTHTGSQK |
| | PFQCRICMRNFSRNFILQRHTRTHTGEKPFQCRIC |
| | MRNFSRKDDLKRHLRTHLRGSSPKKKRKVGVDG |
| | SSGSETPGTSESATPESTGDSVAFEDVAVNFTLEE |
| | WALLDPSQKNLYRDVMRETFRNLASVGKQWED |
| | QNIEDPFKIPRRNISHIPERLCESKEGGQGEESADY |
| | KDDDDKAPKKKRKVPKKKRKV (SEQ ID NO: |
| | 713) |
| ZF #1 + ZF #3 | AGCTGGTGAAGAACTGCTTCCTGCCACTGCGG |
| (Distal, Disordered) | GAGTACTTCAAGTATTTTTCCACCGAGCTGACA |
| nucleic acid | TCTAGCCTGGGAGGACCCTCCTCTGGCGCCCCA |
| | CCACCTAGCGGCGGCTCCCCTGCCGGCTCTCCA |
| | ACCAGCACAGAGGAGGGCACCAGCGAGTCCGC |
| | CACACCAGAGTCTGGACCTGGCACCAGCACAG |
| | AGCCATCCGAGGGCTCTGCCCCAGGCTCTCCTG |
| | CAGGCAGCCCTACCTCCACCGAAGAGGGCACC |
| | AGCACAGAGCCTTCTGAGGGCAGCGCCCCAGG |
| | CACCTCTACAGAGCCAAGCGAGTCCCGGCCAG |
| | GGGAACGGCCCTTCCAGTGTCGGATCTGCATG |
| | AGAAACTTTTCAAGAACAGACACTCTGGCCCG |
| | ACACCTGAGGACCCACACTGGAGAGAAACCCT |
| | TTCAGTGCAGGATATGTATGCGGAATTTTTCCA |
| | GAAAGACCGCACTTAATCGACATCTCAAGACA |
| | CATACCGGGAGTCAGAAGCCTTTCCAATGCCG |
| | GATTTGCATGAGGAACTTCTCCCGGAACGAATC |
| | TCTGAAGGTGCACCTGCGCACACATACAGGCG |
| | AGAAGCCATTCCAGTGTAGGATCTGCATGCGC |
| | AATTTTAGCGTGAAGAATACACTGACGAGACA |
| | TCTGAAGACGCATACAGGTTCCCAGAAGCCCTT |
| | TCAGTGCAGGATCTGCATGAGGAATTTTAGTAG |
| | AAGGGAGCATCTCGTTAGACACCTCCGGACGC |
| | ATACTGGAGAGAAGCCCTTTCAGTGTAGGATTT |
| | GTATGCGGAACTTCAGCATAAGCCACAATCTG |
| | GCAAGGCACCTTAAAACTCATTTGCGCGGGTCT |
| | GGTGTTGATGGCTCCTCTGGTTCTGAAACTCCT |
| | GGTACTTCTGAGTCTGCTACTCCCGAATCTTCC |
| | CGGCCAGGGGAACGGCCCTTCCAGTGTCGGAT |
| | CTGCATGAGAAACTTTTCACGCAGACAGCACCT |
| | GACACTCCACACTCGAACCCACACTGGAGAGA |
| | AACCCTTTCAGTGCAGGATATGTATGCGGAATT |
| | TTTCCGACAACAGCCACCTGCAAAGGCATCTCC |
| | GCACACATACCGGGAGTCAGAAGCCTTTCCAA |
| | TGCCGGATTTGCATGAGGAACTTCTCCCTTCCC |
| | CACCATCTGCAGCGCCACCTGCGGACACATAC |
| | AGGCGAGAAGCCATTCCAGTGTAGGATCTGCA |
| | TGCGCAATTTTAGCCAGTCTAATAACCTTACTC |
| | GCCACCTCAAGACGCATACAGGTTCCCAGAAG |
| | CCCTTTCAGTGCAGGATCTGCATGAGGAATTTT |
| | AGTAGGAACTTCATACTGCAACGGCACACAAG |
| | AACGCATACTGGAGAGAAGCCCTTTCAGTGTA |
| | GGATTTGTATGCGGAACTTCAGCAGGAAGGAC |
| | GATCTGAAAAGACATCTGAGAACTCATTTGCG |
| | CGGGTCTAGCCCCAAGAAGAAGAGAAAGGTGG |
| | GAGTCGACGGATCCAGCGGCTCCGAGACCCCA |
| | GGCACATCTGAGAGCGCCACCCCTGAGTCCAC |
| | CGGTGACTCCGTTGCTTTCGAGGACGTGGCCGT |
| | GAACTTCACACTTGAGGAATGGGCCTTGCTCGA |
| | CCCAAGTCAGAAGAATCTGTACAGAGACGTGA |
| | TGCGGGAGACATTCAGGAATCTCGCCAGTGTC |
| | GGAAAGCAGTGGGAAGACCAGAACATCGAAG |
| | ATCCTTTCAAGATACCACGGCGCAATATCTCCC |
| | ACATTCCTGAGAGGCTGTGTGAATCTAAGGAA |
| | GGCGGACAAGGTGAGGAAAGCGCTGATTACAA |
| | AGATGATGACGATAAAGCCCCCAAGAAGAAAA |
| | GGAAGGTCCCAAAGAAAAAAAGAAAGGTGTG |
| | A (SEQ ID NO: 714) |
| ZF #1 + ZF #3 | TCTAGCCTGGGAGGACCCTCCTCTGGCGCCCCA |
| (Distal, Disordered) | CCACCTAGCGGCGGCTCCCCTGCCGGCTCTCCA |
| full plasmid | ACCAGCACAGAGGAGGGCACCAGCGAGTCCGC |
| | CACACCAGAGTCTGGACCTGGCACCAGCACAG |
| | AGCCATCCGAGGGCTCTGCCCCAGGCTCTCCTG |
| | CAGGCAGCCCTACCTCCACCGAAGAGGGCACC |
| | AGCACAGAGCCTTCTGAGGGCAGCGCCCCAGG |
| | CACCTCTACAGAGCCAAGCGAGTCCCGGCCAG |
| | GGGAACGGCCCTTCCAGTGTCGGATCTGCATG |
| | AGAAACTTTTCAAGAACAGACACTCTGGCCCG |
| | ACACCTGAGGACCCACACTGGAGAGAAACCCT |
| | TTCAGTGCAGGATATGTATGCGGAATTTTTCCA |
| | GAAAGACCGCACTTAATCGACATCTCAAGACA |
| | CATACCGGGAGTCAGAAGCCTTTCCAATGCCG |
| | GATTTGCATGAGGAACTTCTCCCGGAACGAATC |
| | TCTGAAGGTGCACCTGCGCACACATACAGGCG |
| | AGAAGCCATTCCAGTGTAGGATCTGCATGCGC |
| | AATTTTAGCGTGAAGAATACACTGACGAGACA |
| | TCTGAAGACGCATACAGGTTCCCAGAAGCCCTT |
| | TCAGTGCAGGATCTGCATGAGGAATTTTAGTAG |
| | AAGGGAGCATCTCGTTAGACACCTCCGGACGC |
| | ATACTGGAGAGAAGCCCTTTCAGTGTAGGATTT |
| | GTATGCGGAACTTCAGCATAAGCCACAATCTG |
| | GCAAGGCACCTTAAAACTCATTTGCGCGGGTCT |
| | GGTGTTGATGGCTCCTCTGGTTCTGAAACTCCT |
| | GGTACTTCTGAGTCTGCTACTCCCGAATCTTCC |
| | CGGCCAGGGGAACGGCCCTTCCAGTGTCGGAT |
| | CTGCATGAGAAACTTTTCACGCAGACAGCACCT |
| | GACACTCCACACTCGAACCCACACTGGAGAGA |
| | AACCCTTTCAGTGCAGGATATGTATGCGGAATT |
| | TTTCCGACAACAGCCACCTGCAAAGGCATCTCC |
| | GCACACATACCGGGAGTCAGAAGCCTTTCCAA |
| | TGCCGGATTTGCATGAGGAACTTCTCCCTTCCC |
| | CACCATCTGCAGCGCCACCTGCGGACACATAC |
| | AGGCGAGAAGCCATTCCAGTGTAGGATCTGCA |
| | TGCGCAATTTTAGCCAGTCTAATAACCTTACTC |
| | GCCACCTCAAGACGCATACAGGTTCCCAGAAG |
| | CCCTTTCAGTGCAGGATCTGCATGAGGAATTTT |
| | AGTAGGAACTTCATACTGCAACGGCACACAAG |
| | AACGCATACTGGAGAGAAGCCCTTTCAGTGTA |
| | GGATTTGTATGCGGAACTTCAGCAGGAAGGAC |
| | GATCTGAAAAGACATCTGAGAACTCATTTGCG |
| | CGGGTCTAGCCCCAAGAAGAAGAGAAAGGTGG |
| | GAGTCGACGGATCCAGCGGCTCCGAGACCCCA |
| | GGCAC (SEQ ID NO: 715) |
| ZF #3 + ZF #1 | MPKKKRKVPKKKRKVYNHDQEFDPPKVYPPVPA |
| (Distal, Ordered) | EKRKPIRVLSLFDGIATGLLVLKDLGIQVDRYIAS |
| amino acid | EVCEDSITVGMVRHQGKIMYVGDVRSVTQKHIQ |
| | EWGPFDLVIGGSPCNDLSIVNPARKGLYEGTGRL |
| | FFEFYRLLHDARPKEGDDRPFFWLFENVVAMGV |
| | SDKRDISRFLESNPVMIDAKEVSAAHRARYFWGN |
| | LPGMNRPLASTVNDKLELQECLEHGRIAKFSKVR |
| | TITTRSNSIKQGKDQHFPVFMNEKEDILWCTEME |
| | RVFGFPVHYTDVSNMSRLARQRLLGRSWSVPVIR |
| | HLFAPLKEYFACVSSGNSNANSRGPSFSSGLVPLS |
| | LRGSHMAAIPALDPEAEPSMDVILVGSSELSSSVS |
| | PGTGRDLIAYEVKANQRNIEDICICCGSLQVHTQH |
| | PLFEGGICAPCKDKFLDALFLYDDDGYQSYCSICC |
| | SGETLLICGNPDCTRCYCFECVDSLVGPGTSGKV |
| | HAMSNWVCYLCLPSSRSGLLQRRRKWRSQLKAF |
| | YDRESENPLEMFETVPVWRRQPVRVLSLFEDIKK |
| | ELTSLGFLESGSDPGQLKHVVDVTDTVRKDVEE |
| | WGPFDLVYGATPPLGHTCDRPPSWYLFQFHRLL |
| | QYARPKPGSPRPFFWMFVDNLVLNKEDLDVASR |
| | FLEMEPVTIPDVHGGSLQNAVRVWSNIPAIRSRH |
| | WALVSEEELSLLAQNKQSSKLAAKWPTKLVKNC |
| | FLPLREYFKYFSTELTSSLGGPSSGAPPPSGGSPAG |
| | SPTSTEEGTSESATPESGPGTSTEPSEGSAPGSPAG |
| | SPTSTEEGTSTEPSEGSAPGTSTEPSESRPGERPFQ |
| | CRICMRNFSRRQHLTLHTRTHTGEKPFQCRICMR |
| | NFSDNSHLQRHLRTHTGSQKPFQCRICMRNFSLP |
| | HHLQRHLRTHTGEKPFQCRICMRNFSQSNNLTRH |
| | LKTHTGSQKPFQCRICMRNFSRNFILQRHTRTHTG |
| | EKPFQCRICMRNFSRKDDLKRHLRTHLRGSGVDG |
| | SSGSETPGTSESATPESSRPGERPFQCRICMRNFSR |
| | TDTLARHLRTHTGEKPFQCRICMRNFSRKTALNR |
| | HLKTHTGSQKPFQCRICMRNFSRNESLKVHLRTH |
| | TGEKPFQCRICMRNFSVKNTLTRHLKTHTGSQKP |
| | FQCRICMRNFSRREHLVRHLRTHTGEKPFQCRIC |
| | MRNFSISHNLARHLKTHLRGSSPKKKRKVGVDGS |
| | SGSETPGTSESATPESTGDSVAFEDVAVNFTLEEW |
| | ALLDPSQKNLYRDVMRETFRNLASVGKQWEDQ |
| | NIEDPFKIPRRNISHIPERLCESKEGGQGEESADYK |
| | DDDDKAPKKKRKVPKKKRKV (SEQ ID NO: 716) |
| ZF #3 + ZF #1 | TCTGGCGCCCCACCACCTAGCGGCGGCTCCCCT |
| (Distal, Ordered) | GCCGGCTCTCCAACCAGCACAGAGGAGGGCAC |
| nucleic acid | CAGCGAGTCCGCCACACCAGAGTCTGGACCTG |
| | GCACCAGCACAGAGCCATCCGAGGGCTCTGCC |
| | CCAGGCTCTCCTGCAGGCAGCCCTACCTCCACC |
| | GAAGAGGGCACCAGCACAGAGCCTTCTGAGGG |
| | CAGCGCCCCAGGCACCTCTACAGAGCCAAGCG |
| | AGTCCCGGCCAGGGGAACGGCCCTTCCAGTGT |
| | CGGATCTGCATGAGAAACTTTTCACGCAGACA |
| | GCACCTGACACTCCACACTCGAACCCACACTG |
| | GAGAGAAACCCTTTCAGTGCAGGATATGTATG |
| | CGGAATTTTTCCGACAACAGCCACCTGCAAAG |
| | GCATCTCCGCACACATACCGGGAGTCAGAAGC |
| | CTTTCCAATGCCGGATTTGCATGAGGAACTTCT |
| | CCCTTCCCCACCATCTGCAGCGCCACCTGCGGA |
| | CACATACAGGCGAGAAGCCATTCCAGTGTAGG |
| | ATCTGCATGCGCAATTTTAGCCAGTCTAATAAC |
| | CTTACTCGCCACCTCAAGACGCATACAGGTTCC |
| | CAGAAGCCCTTTCAGTGCAGGATCTGCATGAG |
| | GAATTTTAGTAGGAACTTCATACTGCAACGGCA |
| | CACAAGAACGCATACTGGAGAGAAGCCCTTTC |
| | AGTGTAGGATTTGTATGCGGAACTTCAGCAGG |
| | AAGGACGATCTGAAAAGACATCTGAGAACTCA |
| | TTTGCGCGGGTCTGGTGTTGATGGCTCCTCTGG |
| | TTCTGAAACTCCTGGTACTTCTGAGTCTGCTAC |
| | TCCCGAATCTTCCCGGCCAGGGGAACGGCCCTT |
| | CCAGTGTCGGATCTGCATGAGAAACTTTTCAAG |
| | AACAGACACTCTGGCCCGACACCTGAGGACCC |
| | ACACTGGAGAGAAACCCTTTCAGTGCAGGATA |
| | TGTATGCGGAATTTTTCCAGAAAGACCGCACTT |
| | AATCGACATCTCAAGACACATACCGGGAGTCA |
| | GAAGCCTTTCCAATGCCGGATTTGCATGAGGA |
| | ACTTCTCCCGGAACGAATCTCTGAAGGTGCACC |
| | TGCGCACACATACAGGCGAGAAGCCATTCCAG |
| | TGTAGGATCTGCATGCGCAATTTTAGCGTGAAG |
| | AATACACTGACGAGACATCTGAAGACGCATAC |
| | AGGTTCCCAGAAGCCCTTTCAGTGCAGGATCTG |
| | CATGAGGAATTTTAGTAGAAGGGAGCATCTCG |
| | TTAGACACCTCCGGACGCATACTGGAGAGAAG |
| | CCCTTTCAGTGTAGGATTTGTATGCGGAACTTC |
| | AGCATAAGCCACAATCTGGCAAGGCACCTTAA |
| | AACTCATTTGCGCGGGTCTAGCCCCAAGAAGA |
| | AGAGAAAGGTGGGAGTCGACGGATCCAGCGGC |
| | TCCGAGACCCCAGGCACATCTGAGAGCGCCAC |
| | CCCTGAGTCCACCGGTGACTCCGTTGCTTTCGA |
| | GGACGTGGCCGTGAACTTCACACTTGAGGAAT |
| | GGGCCTTGCTCGACCCAAGTCAGAAGAATCTG |
| | TACAGAGACGTGATGCGGGAGACATTCAGGAA |
| | TCTCGCCAGTGTCGGAAAGCAGTGGGAAGACC |
| | AGAACATCGAAGATCCTTTCAAGATACCACGG |
| | CGCAATATCTCCCACATTCCTGAGAGGCTGTGT |
| | GAATCTAAGGAAGGCGGACAAGGTGAGGAAA |
| | GCGCTGATTACAAAGATGATGACGATAAAGCC |
| | CCCAAGAAGAAAAGGAAGGTCCCAAAGAAAA |
| | AAAGAAAGGTGTGA (SEQ ID NO: 716) |
| ZF #3 + ZF #1 | ACCAGCACAGAGGAGGGCACCAGCGAGTCCGC |
| (Distal, Ordered) | CACACCAGAGTCTGGACCTGGCACCAGCACAG |
| full plasmid | AGCCATCCGAGGGCTCTGCCCCAGGCTCTCCTG |
| | CAGGCAGCCCTACCTCCACCGAAGAGGGCACC |
| | AGCACAGAGCCTTCTGAGGGCAGCGCCCCAGG |
| | CACCTCTACAGAGCCAAGCGAGTCCCGGCCAG |
| | GGGAACGGCCCTTCCAGTGTCGGATCTGCATG |
| | AGAAACTTTTCACGCAGACAGCACCTGACACT |
| | CCACACTCGAACCCACACTGGAGAGAAACCCT |
| | TTCAGTGCAGGATATGTATGCGGAATTTTTCCG |
| | ACAACAGCCACCTGCAAAGGCATCTCCGCACA |
| | CATACCGGGAGTCAGAAGCCTTTCCAATGCCG |
| | GATTTGCATGAGGAACTTCTCCCTTCCCCACCA |
| | TCTGCAGCGCCACCTGCGGACACATACAGGCG |
| | AGAAGCCATTCCAGTGTAGGATCTGCATGCGC |
| | AATTTTAGCCAGTCTAATAACCTTACTCGCCAC |
| | CTCAAGACGCATACAGGTTCCCAGAAGCCCTTT |
| | CAGTGCAGGATCTGCATGAGGAATTTTAGTAG |
| | GAACTTCATACTGCAACGGCACACAAGAACGC |
| | ATACTGGAGAGAAGCCCTTTCAGTGTAGGATTT |
| | GTATGCGGAACTTCAGCAGGAAGGACGATCTG |
| | AAAAGACATCTGAGAACTCATTTGCGCGGGTC |
| | TGGTGTTGATGGCTCCTCTGGTTCTGAAACTCC |
| | TGGTACTTCTGAGTCTGCTACTCCCGAATCTTC |
| | CCGGCCAGGGGAACGGCCCTTCCAGTGTCGGA |
| | TCTGCATGAGAAACTTTTCAAGAACAGACACTC |
| | TGGCCCGACACCTGAGGACCCACACTGGAGAG |
| | AAACCCTTTCAGTGCAGGATATGTATGCGGAAT |
| | TTTTCCAGAAAGACCGCACTTAATCGACATCTC |
| | AAGACACATACCGGGAGTCAGAAGCCTTTCCA |
| | ATGCCGGATTTGCATGAGGAACTTCTCCCGGAA |
| | CGAATCTCTGAAGGTGCACCTGCGCACACATA |
| | CAGGCGAGAAGCCATTCCAGTGTAGGATCTGC |
| | ATGCGCAATTTTAGCGTGAAGAATACACTGAC |
| | GAGACATCTGAAGACGCATACAGGTTCCCAGA |
| | AGCCCTTTCAGTGCAGGATCTGCATGAGGAATT |
| | TTAGTAGAAGGGAGCATCTCGTTAGACACCTCC |
| | GGACGCATACTGGAGAGAAGCCCTTTCAGTGT |
| | AGGATTTGTATGCGGAACTTCAGCATAAGCCA |
| | CAATCTGGCAAGGCACCTTAAAACTCATTTGCG |
| | CGGGTCTAGCCCCAAGAAGAAGAGAAAGGTGG |
| | GAGTCGACGGATCCAGCGGCTCCGAGACCCCA |
| | GGCAC (SEQ ID NO: 717) |
| ZF #2 + ZF #3 | MPKKKRKVPKKKRKVYNHDQEFDPPKVYPPVPA |
| (Adjacent, | EKRKPIRVLSLFDGIATGLLVLKDLGIQVDRYIAS |
| Disordered) | EVCEDSITVGMVRHQGKIMYVGDVRSVTQKHIQ |
| amino acid | EWGPFDLVIGGSPCNDLSIVNPARKGLYEGTGRL |
| | FFEFYRLLHDARPKEGDDRPFFWLFENVVAMGV |
| | SDKRDISRFLESNPVMIDAKEVSAAHRARYFWGN |
| | LPGMNRPLASTVNDKLELQECLEHGRIAKFSKVR |
| | TITTRSNSIKQGKDQHFPVFMNEKEDILWCTEME |
| | RVFGFPVHYTDVSNMSRLARQRLLGRSWSVPVIR |
| | HLFAPLKEYFACVSSGNSNANSRGPSFSSGLVPLS |
| | LRGSHMAAIPALDPEAEPSMDVILVGSSELSSSVS |
| | PGTGRDLIAYEVKANQRNIEDICICCGSLQVHTQH |
| | PLFEGGICAPCKDKFLDALFLYDDDGYQSYCSICC |
| | SGETLLICGNPDCTRCYCFECVDSLVGPGTSGKV |
| | HAMSNWVCYLCLPSSRSGLLQRRRKWRSQLKAF |
| | YDRESENPLEMFETVPVWRRQPVRVLSLFEDIKK |
| | ELTSLGFLESGSDPGQLKHVVDVTDTVRKDVEE |
| | WGPFDLVYGATPPLGHTCDRPPSWYLFQFHRLL |
| | QYARPKPGSPRPFFWMFVDNLVLNKEDLDVASR |
| | FLEMEPVTIPDVHGGSLQNAVRVWSNIPAIRSRH |
| | WALVSEEELSLLAQNKQSSKLAAKWPTKLVKNC |
| | FLPLREYFKYFSTELTSSLGGPSSGAPPPSGGSPAG |
| | SPTSTEEGTSESATPESGPGTSTEPSEGSAPGSPAG |
| | SPTSTEEGTSTEPSEGSAPGTSTEPSESRPGERPFQ |
| | CRICMRNFSTPSKLDRHTRTHTGEKPFQCRICMR |
| | NFSLAENLRRHLRTHTGSQKPFQCRICMRNFSRQ |
| | DNLGRHLRTHTGEKPFQCRICMRNFSDGGNLGR |
| | HLKTHTGGGGSQKPFQCRICMRNFSQSPHLKRHL |
| | RTHTGEKPFQCRICMRNFSQSTSLQRHLKTHLRG |
| | SGVDGSSGSETPGTSESATPESSRPGERPFQCRIC |
| | MRNFSRRQHLTLHTRTHTGEKPFQCRICMRNFSD |
| | NSHLQRHLRTHTGSQKPFQCRICMRNFSLPHHLQ |
| | RHLRTHTGEKPFQCRICMRNFSQSNNLTRHLKTH |
| | TGSQKPFQCRICMRNFSRNFILQRHTRTHTGEKPF |
| | QCRICMRNFSRKDDLKRHLRTHLRGSSPKKKRK |
| | VGVDGSSGSETPGTSESATPESTGDSVAFEDVAV |
| | NFTLEEWALLDPSQKNLYRDVMRETFRNLASVG |
| | KQWEDQNIEDPFKIPRRNISHIPERLCESKEGGQG |
| | EESADYKDDDDKAPKKKRKVPKKKRKV (SEQ ID |
| | NO: 718) |
| ZF #2 + ZF #3 | GAGTCTGGACCTGGCACCAGCACAGAGCCATC |
| (Adjacent, | CGAGGGCTCTGCCCCAGGCTCTCCTGCAGGCA |
| Disordered) | GCCCTACCTCCACCGAAGAGGGCACCAGCACA |
| nucleic acid | GAGCCTTCTGAGGGCAGCGCCCCAGGCACCTC |
| | TACAGAGCCAAGCGAGTCCCGGCCAGGGGAAC |
| | GGCCCTTCCAGTGTCGGATCTGCATGAGAAACT |
| | TTTCAACACCCTCAAAGCTGGATAGGCATACAC |
| | GCACCCACACTGGAGAGAAACCCTTTCAGTGC |
| | AGGATATGTATGCGGAATTTTTCCCTGGCTGAG |
| | AACCTCCGAAGGCACCTGAGAACACATACCGG |
| | GAGTCAGAAGCCTTTCCAATGCCGGATTTGCAT |
| | GAGGAACTTCTCCCGCCAAGATAACCTGGGCC |
| | GCCACCTGCGCACACATACAGGCGAGAAGCCA |
| | TTCCAGTGTAGGATCTGCATGCGCAATTTTAGC |
| | GACGGCGGTAACTTGGGACGCCACCTCAAAAC |
| | GCATACAGGTGGCGGTGGCTCCCAGAAGCCCT |
| | TTCAGTGCAGGATCTGCATGAGGAATTTTAGTC |
| | AGTCCCCACATCTTAAAAGACACTTGCGGACG |
| | CATACTGGAGAGAAGCCCTTTCAGTGTAGGATT |
| | TGTATGCGGAACTTCAGCCAGAGCACAAGCCT |
| | GCAACGCCATCTCAAAACTCATTTGCGCGGGTC |
| | TGGTGTTGATGGCTCCTCTGGTTCTGAAACTCC |
| | TGGTACTTCTGAGTCTGCTACTCCCGAATCTTC |
| | CCGGCCAGGGGAACGGCCCTTCCAGTGTCGGA |
| | TCTGCATGAGAAACTTTTCACGCAGACAGCACC |
| | TGACACTCCACACTCGAACCCACACTGGAGAG |
| | AAACCCTTTCAGTGCAGGATATGTATGCGGAAT |
| | TTTTCCGACAACAGCCACCTGCAAAGGCATCTC |
| | CGCACACATACCGGGAGTCAGAAGCCTTTCCA |
| | ATGCCGGATTTGCATGAGGAACTTCTCCCTTCC |
| | CCACCATCTGCAGCGCCACCTGCGGACACATA |
| | CAGGCGAGAAGCCATTCCAGTGTAGGATCTGC |
| | ATGCGCAATTTTAGCCAGTCTAATAACCTTACT |
| | CGCCACCTCAAGACGCATACAGGTTCCCAGAA |
| | GCCCTTTCAGTGCAGGATCTGCATGAGGAATTT |
| | TAGTAGGAACTTCATACTGCAACGGCACACAA |
| | GAACGCATACTGGAGAGAAGCCCTTTCAGTGT |
| | AGGATTTGTATGCGGAACTTCAGCAGGAAGGA |
| | CGATCTGAAAAGACATCTGAGAACTCATTTGC |
| | GCGGGTCTAGCCCCAAGAAGAAGAGAAAGGTG |
| | GGAGTCGACGGATCCAGCGGCTCCGAGACCCC |
| | AGGCACATCTGAGAGCGCCACCCCTGAGTCCA |
| | CCGGTGACTCCGTTGCTTTCGAGGACGTGGCCG |
| | TGAACTTCACACTTGAGGAATGGGCCTTGCTCG |
| | ACCCAAGTCAGAAGAATCTGTACAGAGACGTG |
| | ATGCGGGAGACATTCAGGAATCTCGCCAGTGT |
| | CGGAAAGCAGTGGGAAGACCAGAACATCGAA |
| | GATCCTTTCAAGATACCACGGCGCAATATCTCC |
| | CACATTCCTGAGAGGCTGTGTGAATCTAAGGA |
| | AGGCGGACAAGGTGAGGAAAGCGCTGATTACA |
| | AAGATGATGACGATAAAGCCCCCAAGAAGAAA |
| | AGGAAGGTCCCAAAGAAAAAAAGAAAGGTGT |
| | GA (SEQ ID NO: 719) |
| ZF #2 + ZF #3 | GCCATCCGAGGGCTCTGCCCCAGGCTCTCCTGC |
| (Adjacent, | AGGCAGCCCTACCTCCACCGAAGAGGGCACCA |
| Disordered) | GCACAGAGCCTTCTGAGGGCAGCGCCCCAGGC |
| full plasmid | ACCTCTACAGAGCCAAGCGAGTCCCGGCCAGG |
| | GGAACGGCCCTTCCAGTGTCGGATCTGCATGA |
| | GAAACTTTTCAACACCCTCAAAGCTGGATAGG |
| | CATACACGCACCCACACTGGAGAGAAACCCTT |
| | TCAGTGCAGGATATGTATGCGGAATTTTTCCCT |
| | GGCTGAGAACCTCCGAAGGCACCTGAGAACAC |
| | ATACCGGGAGTCAGAAGCCTTTCCAATGCCGG |
| | ATTTGCATGAGGAACTTCTCCCGCCAAGATAAC |
| | CTGGGCCGCCACCTGCGCACACATACAGGCGA |
| | GAAGCCATTCCAGTGTAGGATCTGCATGCGCA |
| | ATTTTAGCGACGGCGGTAACTTGGGACGCCAC |
| | CTCAAAACGCATACAGGTGGCGGTGGCTCCCA |
| | GAAGCCCTTTCAGTGCAGGATCTGCATGAGGA |
| | ATTTTAGTCAGTCCCCACATCTTAAAAGACACT |
| | TGCGGACGCATACTGGAGAGAAGCCCTTTCAG |
| | TGTAGGATTTGTATGCGGAACTTCAGCCAGAGC |
| | ACAAGCCTGCAACGCCATCTCAAAACTCATTTG |
| | CGCGGGTCTGGTGTTGATGGCTCCTCTGGTTCT |
| | GAAACTCCTGGTACTTCTGAGTCTGCTACTCCC |
| | GAATCTTCCCGGCCAGGGGAACGGCCCTTCCA |
| | GTGTCGGATCTGCATGAGAAACTTTTCACGCAG |
| | ACAGCACCTGACACTCCACACTCGAACCCACA |
| | CTGGAGAGAAACCCTTTCAGTGCAGGATATGT |
| | ATGCGGAATTTTTCCGACAACAGCCACCTGCAA |
| | AGGCATCTCCGCACACATACCGGGAGTCAGAA |
| | GCCTTTCCAATGCCGGATTTGCATGAGGAACTT |
| | CTCCCTTCCCCACCATCTGCAGCGCCACCTGCG |
| | GACACATACAGGCGAGAAGCCATTCCAGTGTA |
| | GGATCTGCATGCGCAATTTTAGCCAGTCTAATA |
| | ACCTTACTCGCCACCTCAAGACGCATACAGGTT |
| | CCCAGAAGCCCTTTCAGTGCAGGATCTGCATGA |
| | GGAATTTTAGTAGGAACTTCATACTGCAACGGC |
| | ACACAAGAACGCATACTGGAGAGAAGCCCTTT |
| | CAGTGTAGGATTTGTATGCGGAACTTCAGCAG |
| | GAAGGACGATCTGAAAAGACATCTGAGAACTC |
| | ATTTGCGCGGGTCTAGCCCCAAGAAGAAGAGA |
| | AAGGTGGGAGTCGACGGATCCAGCGGCTCCGA |
| | GACCCCAGGCAC (SEQ ID NO: 720) |
| ZF #3 + ZF #2 | MPKKKRKVPKKKRKVYNHDQEFDPPKVYPPVPA |
| (Adjacent, Ordered) | EKRKPIRVLSLFDGIATGLLVLKDLGIQVDRYIAS |
| amino acid | EVCEDSITVGMVRHQGKIMYVGDVRSVTQKHIQ |
| | EWGPFDLVIGGSPCNDLSIVNPARKGLYEGTGRL |
| | FFEFYRLLHDARPKEGDDRPFFWLFENVVAMGV |
| | SDKRDISRFLESNPVMIDAKEVSAAHRARYFWGN |
| | LPGMNRPLASTVNDKLELQECLEHGRIAKFSKVR |
| | TITTRSNSIKQGKDQHFPVFMNEKEDILWCTEME |
| | RVFGFPVHYTDVSNMSRLARQRLLGRSWSVPVIR |
| | HLFAPLKEYFACVSSGNSNANSRGPSFSSGLVPLS |
| | LRGSHMAAIPALDPEAEPSMDVILVGSSELSSSVS |
| | PGTGRDLIAYEVKANQRNIEDICICCGSLQVHTQH |
| | PLFEGGICAPCKDKFLDALFLYDDDGYQSYCSICC |
| | SGETLLICGNPDCTRCYCFECVDSLVGPGTSGKV |
| | HAMSNWVCYLCLPSSRSGLLQRRRKWRSQLKAF |
| | YDRESENPLEMFETVPVWRRQPVRVLSLFEDIKK |
| | ELTSLGFLESGSDPGQLKHVVDVTDTVRKDVEE |
| | WGPFDLVYGATPPLGHTCDRPPSWYLFQFHRLL |
| | QYARPKPGSPRPFFWMFVDNLVLNKEDLDVASR |
| | FLEMEPVTIPDVHGGSLQNAVRVWSNIPAIRSRH |
| | WALVSEEELSLLAQNKQSSKLAAKWPTKLVKNC |
| | FLPLREYFKYFSTELTSSLGGPSSGAPPPSGGSPAG |
| | SPTSTEEGTSESATPESGPGTSTEPSEGSAPGSPAG |
| | SPTSTEEGTSTEPSEGSAPGTSTEPSESRPGERPFQ |
| | CRICMRNFSRRQHLTLHTRTHTGEKPFQCRICMR |
| | NFSDNSHLQRHLRTHTGSQKPFQCRICMRNFSLP |
| | HHLQRHLRTHTGEKPFQCRICMRNFSQSNNLTRH |
| | LKTHTGSQKPFQCRICMRNFSRNFILQRHTRTHTG |
| | EKPFQCRICMRNFSRKDDLKRHLRTHLRGSGVDG |
| | SSGSETPGTSESATPESSRPGERPFQCRICMRNFST |
| | PSKLDRHTRTHTGEKPFQCRICMRNFSLAENLRR |
| | HLRTHTGSQKPFQCRICMRNFSRQDNLGRHLRTH |
| | TGEKPFQCRICMRNFSDGGNLGRHLKTHTGGGG |
| | SQKPFQCRICMRNFSQSPHLKRHLRTHTGEKPFQ |
| | CRICMRNFSQSTSLQRHLKTHLRGSSPKKKRKVG |
| | VDGSSGSETPGTSESATPESTGDSVAFEDVAVNFT |
| | LEEWALLDPSQKNLYRDVMRETFRNLASVGKQ |
| | WEDQNIEDPFKIPRRNISHIPERLCESKEGGQGEES |
| | ADYKDDDDKAPKKKRKVPKKKRKV (SEQ ID |
| | NO: 721) |
| ZF #3 + ZF #2 | GGCACCAGCACAGAGCCTTCTGAGGGCAGCGC |
| (Adjacent, Ordered) | CCCAGGCACCTCTACAGAGCCAAGCGAGTCCC |
| nucleic acid | GGCCAGGGGAACGGCCCTTCCAGTGTCGGATC |
| | TGCATGAGAAACTTTTCACGCAGACAGCACCT |
| | GACACTCCACACTCGAACCCACACTGGAGAGA |
| | AACCCTTTCAGTGCAGGATATGTATGCGGAATT |
| | TTTCCGACAACAGCCACCTGCAAAGGCATCTCC |
| | GCACACATACCGGGAGTCAGAAGCCTTTCCAA |
| | TGCCGGATTTGCATGAGGAACTTCTCCCTTCCC |
| | CACCATCTGCAGCGCCACCTGCGGACACATAC |
| | AGGCGAGAAGCCATTCCAGTGTAGGATCTGCA |
| | TGCGCAATTTTAGCCAGTCTAATAACCTTACTC |
| | GCCACCTCAAGACGCATACAGGTTCCCAGAAG |
| | CCCTTTCAGTGCAGGATCTGCATGAGGAATTTT |
| | AGTAGGAACTTCATACTGCAACGGCACACAAG |
| | AACGCATACTGGAGAGAAGCCCTTTCAGTGTA |
| | GGATTTGTATGCGGAACTTCAGCAGGAAGGAC |
| | GATCTGAAAAGACATCTGAGAACTCATTTGCG |
| | CGGGTCTGGTGTTGATGGCTCCTCTGGTTCTGA |
| | AACTCCTGGTACTTCTGAGTCTGCTACTCCCGA |
| | ATCTTCCCGGCCAGGGGAACGGCCCTTCCAGTG |
| | TCGGATCTGCATGAGAAACTTTTCAACACCCTC |
| | AAAGCTGGATAGGCATACACGCACCCACACTG |
| | GAGAGAAACCCTTTCAGTGCAGGATATGTATG |
| | CGGAATTTTTCCCTGGCTGAGAACCTCCGAAGG |
| | CACCTGAGAACACATACCGGGAGTCAGAAGCC |
| | TTTCCAATGCCGGATTTGCATGAGGAACTTCTC |
| | CCGCCAAGATAACCTGGGCCGCCACCTGCGCA |
| | CACATACAGGCGAGAAGCCATTCCAGTGTAGG |
| | ATCTGCATGCGCAATTTTAGCGACGGCGGTAAC |
| | TTGGGACGCCACCTCAAAACGCATACAGGTGG |
| | CGGTGGCTCCCAGAAGCCCTTTCAGTGCAGGAT |
| | CTGCATGAGGAATTTTAGTCAGTCCCCACATCT |
| | TAAAAGACACTTGCGGACGCATACTGGAGAGA |
| | AGCCCTTTCAGTGTAGGATTTGTATGCGGAACT |
| | TCAGCCAGAGCACAAGCCTGCAACGCCATCTC |
| | AAAACTCATTTGCGCGGGTCTAGCCCCAAGAA |
| | GAAGAGAAAGGTGGGAGTCGACGGATCCAGCG |
| | GCTCCGAGACCCCAGGCACATCTGAGAGCGCC |
| | ACCCCTGAGTCCACCGGTGACTCCGTTGCTTTC |
| | GAGGACGTGGCCGTGAACTTCACACTTGAGGA |
| | ATGGGCCTTGCTCGACCCAAGTCAGAAGAATC |
| | TGTACAGAGACGTGATGCGGGAGACATTCAGG |
| | AATCTCGCCAGTGTCGGAAAGCAGTGGGAAGA |
| | CCAGAACATCGAAGATCCTTTCAAGATACCAC |
| | GGCGCAATATCTCCCACATTCCTGAGAGGCTGT |
| | GTGAATCTAAGGAAGGCGGACAAGGTGAGGAA |
| | AGCGCTGATTACAAAGATGATGACGATAAAGC |
| | CCCCAAGAAGAAAAGGAAGGTCCCAAAGAAA |
| | AAAAGAAAGGTGTGA (SEQ ID NO: 722) |
| ZF #3 + ZF #2 | GCACAGAGCCTTCTGAGGGCAGCGCCCCAGGC |
| (Adjacent, Ordered) | ACCTCTACAGAGCCAAGCGAGTCCCGGCCAGG |
| full plasmid | GGAACGGCCCTTCCAGTGTCGGATCTGCATGA |
| | GAAACTTTTCACGCAGACAGCACCTGACACTCC |
| | ACACTCGAACCCACACTGGAGAGAAACCCTTT |
| | CAGTGCAGGATATGTATGCGGAATTTTTCCGAC |
| | AACAGCCACCTGCAAAGGCATCTCCGCACACA |
| | TACCGGGAGTCAGAAGCCTTTCCAATGCCGGA |
| | TTTGCATGAGGAACTTCTCCCTTCCCCACCATC |
| | TGCAGCGCCACCTGCGGACACATACAGGCGAG |
| | AAGCCATTCCAGTGTAGGATCTGCATGCGCAAT |
| | TTTAGCCAGTCTAATAACCTTACTCGCCACCTC |
| | AAGACGCATACAGGTTCCCAGAAGCCCTTTCA |
| | GTGCAGGATCTGCATGAGGAATTTTAGTAGGA |
| | ACTTCATACTGCAACGGCACACAAGAACGCAT |
| | ACTGGAGAGAAGCCCTTTCAGTGTAGGATTTGT |
| | ATGCGGAACTTCAGCAGGAAGGACGATCTGAA |
| | AAGACATCTGAGAACTCATTTGCGCGGGTCTG |
| | GTGTTGATGGCTCCTCTGGTTCTGAAACTCCTG |
| | GTACTTCTGAGTCTGCTACTCCCGAATCTTCCC |
| | GGCCAGGGGAACGGCCCTTCCAGTGTCGGATC |
| | TGCATGAGAAACTTTTCAACACCCTCAAAGCTG |
| | GATAGGCATACACGCACCCACACTGGAGAGAA |
| | ACCCTTTCAGTGCAGGATATGTATGCGGAATTT |
| | TTCCCTGGCTGAGAACCTCCGAAGGCACCTGA |
| | GAACACATACCGGGAGTCAGAAGCCTTTCCAA |
| | TGCCGGATTTGCATGAGGAACTTCTCCCGCCAA |
| | GATAACCTGGGCCGCCACCTGCGCACACATAC |
| | AGGCGAGAAGCCATTCCAGTGTAGGATCTGCA |
| | TGCGCAATTTTAGCGACGGCGGTAACTTGGGA |
| | CGCCACCTCAAAACGCATACAGGTGGCGGTGG |
| | CTCCCAGAAGCCCTTTCAGTGCAGGATCTGCAT |
| | GAGGAATTTTAGTCAGTCCCCACATCTTAAAAG |
| | ACACTTGCGGACGCATACTGGAGAGAAGCCCT |
| | TTCAGTGTAGGATTTGTATGCGGAACTTCAGCC |
| | AGAGCACAAGCCTGCAACGCCATCTCAAAACT |
| | CATTTGCGCGGGTCTAGCCCCAAGAAGAAGAG |
| | AAAGGTGGGAGTCGACGGATCCAGCGGCTCCG |
| | AGACCCCAGGCAC (SEQ ID NO: 723) |
| ZF #2 + ZF #2 | MPKKKRKVPKKKRKVYNHDQEFDPPKVYPPVPA |
| (Redundant) | EKRKPIRVLSLFDGIATGLLVLKDLGIQVDRYIAS |
| amino acid | EVCEDSITVGMVRHQGKIMYVGDVRSVTQKHIQ |
| | EWGPFDLVIGGSPCNDLSIVNPARKGLYEGTGRL |
| | FFEFYRLLHDARPKEGDDRPFFWLFENVVAMGV |
| | SDKRDISRFLESNPVMIDAKEVSAAHRARYFWGN |
| | LPGMNRPLASTVNDKLELQECLEHGRIAKFSKVR |
| | TITTRSNSIKQGKDQHFPVFMNEKEDILWCTEME |
| | RVFGFPVHYTDVSNMSRLARQRLLGRSWSVPVIR |
| | HLFAPLKEYFACVSSGNSNANSRGPSFSSGLVPLS |
| | LRGSHMAAIPALDPEAEPSMDVILVGSSELSSSVS |
| | PGTGRDLIAYEVKANQRNIEDICICCGSLQVHTQH |
| | PLFEGGICAPCKDKFLDALFLYDDDGYQSYCSICC |
| | SGETLLICGNPDCTRCYCFECVDSLVGPGTSGKV |
| | HAMSNWVCYLCLPSSRSGLLQRRRKWRSQLKAF |
| | YDRESENPLEMFETVPVWRRQPVRVLSLFEDIKK |
| | ELTSLGFLESGSDPGQLKHVVDVTDTVRKDVEE |
| | WGPFDLVYGATPPLGHTCDRPPSWYLFQFHRLL |
| | QYARPKPGSPRPFFWMFVDNLVLNKEDLDVASR |
| | FLEMEPVTIPDVHGGSLQNAVRVWSNIPAIRSRH |
| | WALVSEEELSLLAQNKQSSKLAAKWPTKLVKNC |
| | FLPLREYFKYFSTELTSSLGGPSSGAPPPSGGSPAG |
| | SPTSTEEGTSESATPESGPGTSTEPSEGSAPGSPAG |
| | SPTSTEEGTSTEPSEGSAPGTSTEPSESRPGERPFQ |
| | CRICMRNFSTPSKLDRHTRTHTGEKPFQCRICMR |
| | NFSLAENLRRHLRTHTGSQKPFQCRICMRNFSRQ |
| | DNLGRHLRTHTGEKPFQCRICMRNFSDGGNLGR |
| | HLKTHTGGGGSQKPFQCRICMRNFSQSPHLKRHL |
| | RTHTGEKPFQCRICMRNFSQSTSLQRHLKTHLRG |
| | SGVDGSSGSETPGTSESATPESSRPGERPFQCRIC |
| | MRNFSTPSKLDRHTRTHTGEKPFQCRICMRNFSL |
| | AENLRRHLRTHTGSQKPFQCRICMRNFSRQDNLG |
| | RHLRTHTGEKPFQCRICMRNFSDGGNLGRHLKTH |
| | TGGGGSQKPFQCRICMRNFSQSPHLKRHLRTHTG |
| | EKPFQCRICMRNFSQSTSLQRHLKTHLRGSSPKK |
| | KRKVGVDGSSGSETPGTSESATPESTGDSVAFED |
| | VAVNFTLEEWALLDPSQKNLYRDVMRETFRNLA |
| | SVGKQWEDQNIEDPFKIPRRNISHIPERLCESKEG |
| | GQGEESADYKDDDDKAPKKKRKVPKKKRKV |
| | (SEQ ID NO: 724) |
| ZF #2 + ZF #2 | AGTGCAGGATATGTATGCGGAATTTTTCCCTGG |
| (Redundant) | CTGAGAACCTCCGAAGGCACCTGAGAACACAT |
| nucleic acid | ACCGGGAGTCAGAAGCCTTTCCAATGCCGGAT |
| | TTGCATGAGGAACTTCTCCCGCCAAGATAACCT |
| | GGGCCGCCACCTGCGCACACATACAGGCGAGA |
| | AGCCATTCCAGTGTAGGATCTGCATGCGCAATT |
| | TTAGCGACGGCGGTAACTTGGGACGCCACCTC |
| | AAAACGCATACAGGTGGCGGTGGCTCCCAGAA |
| | GCCCTTTCAGTGCAGGATCTGCATGAGGAATTT |
| | TAGTCAGTCCCCACATCTTAAAAGACACTTGCG |
| | GACGCATACTGGAGAGAAGCCCTTTCAGTGTA |
| | GGATTTGTATGCGGAACTTCAGCCAGAGCACA |
| | AGCCTGCAACGCCATCTCAAAACTCATTTGCGC |
| | GGGTCTGGTGTTGATGGCTCCTCTGGTTCTGAA |
| | ACTCCTGGTACTTCTGAGTCTGCTACTCCCGAA |
| | TCTTCCCGGCCAGGGGAACGGCCCTTCCAGTGT |
| | CGGATCTGCATGAGAAACTTTTCAACACCCTCA |
| | AAGCTGGATAGGCATACACGCACCCACACTGG |
| | AGAGAAACCCTTTCAGTGCAGGATATGTATGC |
| | GGAATTTTTCCCTGGCTGAGAACCTCCGAAGGC |
| | ACCTGAGAACACATACCGGGAGTCAGAAGCCT |
| | TTCCAATGCCGGATTTGCATGAGGAACTTCTCC |
| | CGCCAAGATAACCTGGGCCGCCACCTGCGCAC |
| | ACATACAGGCGAGAAGCCATTCCAGTGTAGGA |
| | TCTGCATGCGCAATTTTAGCGACGGCGGTAACT |
| | TGGGACGCCACCTCAAAACGCATACAGGTGGC |
| | GGTGGCTCCCAGAAGCCCTTTCAGTGCAGGATC |
| | TGCATGAGGAATTTTAGTCAGTCCCCACATCTT |
| | AAAAGACACTTGCGGACGCATACTGGAGAGAA |
| | GCCCTTTCAGTGTAGGATTTGTATGCGGAACTT |
| | CAGCCAGAGCACAAGCCTGCAACGCCATCTCA |
| | AAACTCATTTGCGCGGGTCTAGCCCCAAGAAG |
| | AAGAGAAAGGTGGGAGTCGACGGATCCAGCGG |
| | CTCCGAGACCCCAGGCACATCTGAGAGCGCCA |
| | CCCCTGAGTCCACCGGTGACTCCGTTGCTTTCG |
| | AGGACGTGGCCGTGAACTTCACACTTGAGGAA |
| | TGGGCCTTGCTCGACCCAAGTCAGAAGAATCT |
| | GTACAGAGACGTGATGCGGGAGACATTCAGGA |
| | ATCTCGCCAGTGTCGGAAAGCAGTGGGAAGAC |
| | CAGAACATCGAAGATCCTTTCAAGATACCACG |
| | GCGCAATATCTCCCACATTCCTGAGAGGCTGTG |
| | TGAATCTAAGGAAGGCGGACAAGGTGAGGAAA |
| | GCGCTGATTACAAAGATGATGACGATAAAGCC |
| | CCCAAGAAGAAAAGGAAGGTCCCAAAGAAAA |
| | AAAGAAAGGTGTGA (SEQ ID NO: 726) |
| ZF #2 + ZF #2 | CACGCACCCACACTGGAGAGAAACCCTTTCAG |
| (Redundant) | TGCAGGATATGTATGCGGAATTTTTCCCTGGCT |
| full plasmid | GAGAACCTCCGAAGGCACCTGAGAACACATAC |
| | CGGGAGTCAGAAGCCTTTCCAATGCCGGATTTG |
| | CATGAGGAACTTCTCCCGCCAAGATAACCTGG |
| | GCCGCCACCTGCGCACACATACAGGCGAGAAG |
| | CCATTCCAGTGTAGGATCTGCATGCGCAATTTT |
| | AGCGACGGCGGTAACTTGGGACGCCACCTCAA |
| | AACGCATACAGGTGGCGGTGGCTCCCAGAAGC |
| | CCTTTCAGTGCAGGATCTGCATGAGGAATTTTA |
| | GTCAGTCCCCACATCTTAAAAGACACTTGCGGA |
| | CGCATACTGGAGAGAAGCCCTTTCAGTGTAGG |
| | ATTTGTATGCGGAACTTCAGCCAGAGCACAAG |
| | CCTGCAACGCCATCTCAAAACTCATTTGCGCGG |
| | GTCTGGTGTTGATGGCTCCTCTGGTTCTGAAAC |
| | TCCTGGTACTTCTGAGTCTGCTACTCCCGAATC |
| | TTCCCGGCCAGGGGAACGGCCCTTCCAGTGTCG |
| | GATCTGCATGAGAAACTTTTCAACACCCTCAAA |
| | GCTGGATAGGCATACACGCACCCACACTGGAG |
| | AGAAACCCTTTCAGTGCAGGATATGTATGCGG |
| | AATTTTTCCCTGGCTGAGAACCTCCGAAGGCAC |
| | CTGAGAACACATACCGGGAGTCAGAAGCCTTT |
| | CCAATGCCGGATTTGCATGAGGAACTTCTCCCG |
| | CCAAGATAACCTGGGCCGCCACCTGCGCACAC |
| | ATACAGGCGAGAAGCCATTCCAGTGTAGGATC |
| | TGCATGCGCAATTTTAGCGACGGCGGTAACTTG |
| | GGACGCCACCTCAAAACGCATACAGGTGGCGG |
| | TGGCTCCCAGAAGCCCTTTCAGTGCAGGATCTG |
| | CATGAGGAATTTTAGTCAGTCCCCACATCTTAA |
| | AAGACACTTGCGGACGCATACTGGAGAGAAGC |
| | CCTTTCAGTGTAGGATTTGTATGCGGAACTTCA |
| | GCCAGAGCACAAGCCTGCAACGCCATCTCAAA |
| | ACTCATTTGCGCGGGTCTAGCCCCAAGAAGAA |
| | GAGAAAGGTGGGAGTCGACGGATCCAGCGGCT |
| | CCGAGACCCCAGGCAC (SEQ ID NO: 727) |

Additionally, a control of ZF #2, targeting SEQ ID NO: 5, with amino acid SEQ ID NO: 13 and nucleic acid SEQ ID NO: 14, was used in the dose response experiment.

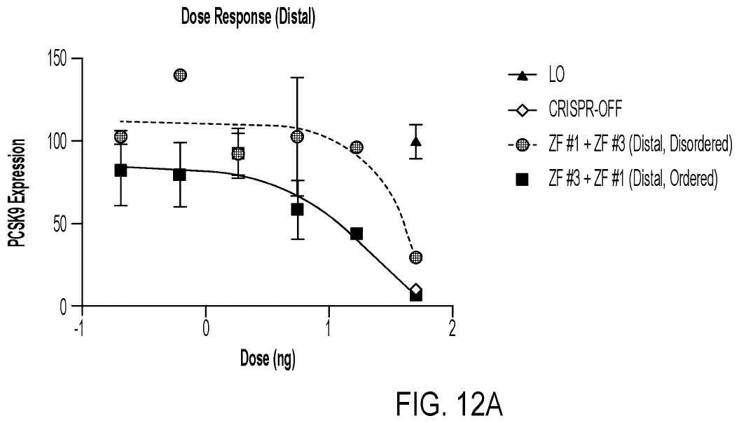

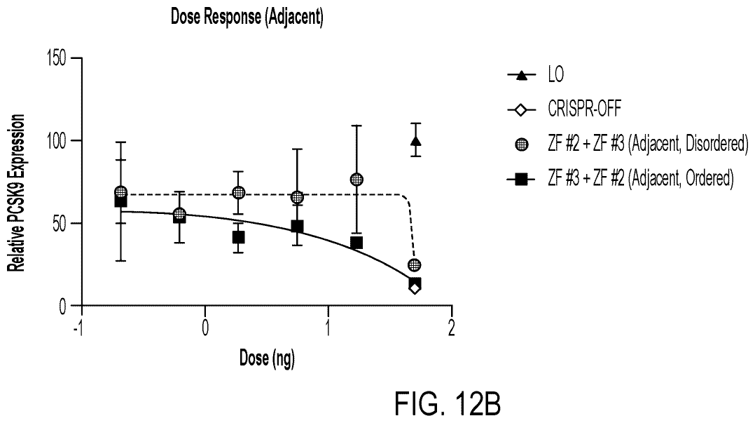

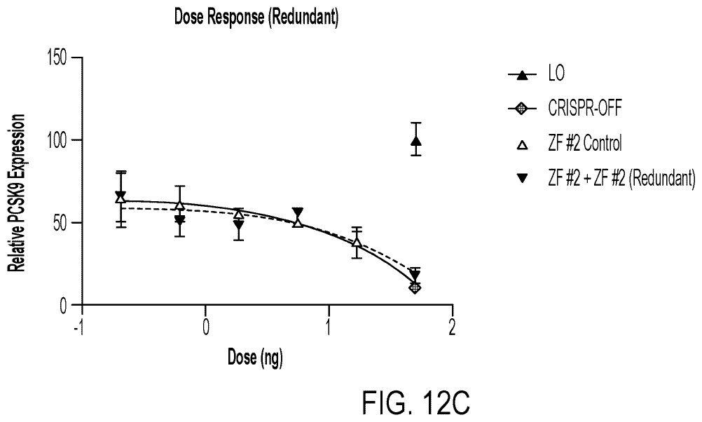

Dose response of the described fusion proteins was tested in Hep3B cells. PCSK9 expression was measured at Day 7. Results are shown in FIG. 12. The data indicate better silencing ability for ordered orientation and demonstrate that the redundant zinc hand construct performs equivalently to the single zinc finger control.

### Sequences

The SEQ ID NOs (SEQ) of nucleotide (nt) and amino acid (aa) sequences described in the present disclosure are listed below. Sequences 1-32 include sequences referred to in the Examples section. Sequences 33-630 disclose exemplary suitable effector domains. Sequences 631-665 disclose exemplary suitable linker sequences.

| SEQ | Description | Sequence |
|---|---|---|
| 1 | ZFP152 (PCSK9 | SRPGERPFQCRICMRNFSTPSKLDRHTRTHTGEKPFQCRICMRNF |
| | DNA-binding zinc | SLAENLRRHLRTHTGSQKPFQCRICMRNFSRQDNLGRHLRTHTGE |
| | finger domain) | KPFQCRICMRNFSDGGNLGRHLKTHTGGGGSQKPFQCRICMRNFS |
| | amino acid sequence | QSPHLKRHLRTHTGEKPFQCRICMRNFSQSTSLQRHLKTHLRGS |
| 2 | ZFP152 nucleotide | TCCCGGCCAGGGGAACGGCCCTTCCAGTGTCGGATCTGCATGAGA |
| | sequence | AACTTTTCAACACCCTCAAAGCTGGATAGGCATACACGCACCCAC |
| | | ACTGGAGAGAAACCCTTTCAGTGCAGGATATGTATGCGGAATTTT |
| | | TCCCTGGCTGAGAACCTCCGAAGGCACCTGAGAACACATACCGGG |
| | | AGTCAGAAGCCTTTCCAATGCCGGATTTGCATGAGGAACTTCTCC |
| | | CGCCAAGATAACCTGGGCCGCCACCTGCGCACACATACAGGCGAG |
| | | AAGCCATTCCAGTGTAGGATCTGCATGCGCAATTTTAGCGACGGC |
| | | GGTAACTTGGGACGCCACCTCAAAACGCATACAGGTGGCGGTGGC |
| | | TCCCAGAAGCCCTTTCAGTGCAGGATCTGCATGAGGAATTTTAGT |
| | | CAGTCCCCACATCTTAAAAGACACTTGCGGACGCATACTGGAGAG |
| | | AAGCCCTTTCAGTGTAGGATTTGTATGCGGAACTTCAGCCAGAGC |
| | | ACAAGCCTGCAACGCCATCTCAAAACTCATTTGCGCGGGTCT |
| 3 | ZFP2244 (CLTA | ERPFQCRICMRNFSHKQHRDAHIRTHTGEKPFACDICGRKFARSA |
| | DNA-binding zinc | NLTRHTKIHTGSQKPFQCRICMRNFSRSDNLSEHIRTHTGEKPFA |
| | finger domain) | CDICGRKFATSANLSRHTKIHTGSQKPFQCRICMRNFSIRSTLRD |
| | amino acid sequence | HIRTHTGEKPFACDICGRKFARTPVRMGHTKIHLRQKDAARGS |
| 4 | ZFP2244 | GAGAGACCTTTTCAGTGCCGGATTTGTATGCGCAATTTCTC |
| | nucleotide coding | CCACAAGCAGCACCGCGACGCCCATATTAGAACACATACTG |
| | sequence | GAGAAAAGCCCTTCGCTTGCGACATCTGTGGACGGAAGTTC |
| | | GCCCGCTCCGCCAACCTGACCCGCCACACTAAAATCCATAC |
| | | CGGCAGCCAAAAGCCATTCCAATGTCGCATCTGTATGCGGA |
| | | ACTTTAGCCGCTCCGACAACCTGTCCGAGCATATTCGGACT |
| | | CACACAGGGGAGAAACCATTTGCATGTGATATCTGCGGCAG |
| | | AAAATTCGCTACCTCCGCCAACCTGTCCCGCCATACCAAGA |
| | | TACACACGGGATCTCAGAAGCCCTTCCAGTGTCGAATCTGC |
| | | ATGCGTAACTTCAGTATCCGCTCCACCCTGCGCGACCACAT |
| | | CCGCACCCACACCGGCGAGAAGCCTTTTGCCTGTGACATTT |
| | | GTGGGAGGAAATTTGCCCGCACCCCGGTGCGCATGGGCCAT |
| | | ACCAAGATACACCTGCGCCAAAAAGATGCGGCCCGGGGATC |
| | | T |
| 5 | ZFP152 target DNA | GCAGGAGGACGAGGACGGC |
| | sequence | |
| 6 | ZFP2244 target | GCTGCCTGATAGGGAGCGTC |
| | DNA sequence | |
| 7 | CLTA gRNA | mG*mC*mA*rArCrArCrCrGrCrCrUrArGrArCrCrGrArCrG |
| | | rUrUrUrArArGrArGrCrUrArArGrCrUrGrGrArArArCrAr |
| | | GrCrArUrArGrCrArArGrUrUrUrArArArUrArArGrGrCrU |
| | | rArGrUrCrCrGrUrUrArUrCrArArCrUrUrGrArArArArAr |
| | | GrUrGrGrCrArCrCrGrArGrUrCrGrGrUrGrCrUrUrUmU* |
| | | mU*mU |
| 8 | PCSK9 gRNA | mA*mC*mU*rGrCrCrUrGrGrCrUrCrArCrUrCrCrUrCrCrG |
| | | rUrUrUrArArGrArGrCrUrArArGrCrUrGrGrArArArCrAr |
| | | GrCrArUrArGrCrArArGrUrUrUrArArArUrArArGrGrCrU |
| | | rArGrUrCrCrGrUrUrArUrCrArArCrUrUrGrArArArArAr |
| | | GrUrGrGrCrArCrC |
| | | rGrArGrUrCrGrGrUrGrCrUrUrU*mU*mU*mU |
| 9 | CRISPRoff Effector | MYPYDVPDYASPKKKRKVNHDQEFDPPKVYPPVPAEKRKPIRVLS |
| | amino acid sequence | LFDGIATGLLVLKDLGIQVDRYIASEVCEDSITVGMVRHQGKIMY |
| | (NLS-hDNMT3A- | VGDVRSVTQKHIQEWGPFDLVIGGSPCNDLSIVNPARKGLYEGTG |
| | W linker- | RLFFEFYRLLHDARPKEGDDRPFFWLFENVVAMGVSDKRDISRFL |
| | hDNMT3L-dCas9- | ESNPVMIDAKEVSAAHRARYFWGNLPGMNRPLASTVNDKLELQEC |
| | NLS-KOX1KRAB) | LEHGRIAKFSKVRTITTRSNSIKQGKDQHFPVFMNEKEDILWCTE |
| | | MERVFGFPVHYTDVSNMSRLARQRLLGRSWSVPVIRHLFAPLKEY |
| | | FACVSSGNSNANSRGPSFSSGLVPLSLRGSHMAAIPALDPEAEPS |
| | | MDVILVGSSELSSSVSPGTGRDLIAYEVKANQRNIEDICICCGSL |
| | | QVHTQHPLFEGGICAPCKDKFLDALFLYDDDGYQSYCSICCSGET |
| | | LLICGNPDCTRCYCFECVDSLVGPGTSGKVHAMSNWVCYLCLPSS |
| | | RSGLLQRRRKWRSQLKAFYDRESENPLEMFETVPVWRRQPVRVLS |
| | | LFEDIKKELTSLGFLESGSDPGQLKHVVDVTDTVRKDVEEWGPFD |
| | | LVYGATPPLGHTCDRPPSWYLFQFHRLLQYARPKPGSPRPFFWMF |
| | | VDNLVLNKEDLDVASRFLEMEPVTIPDVHGGSLQNAVRVWSNIPA |
| | | IRSRHWALVSEEELSLLAQNKQSSKLAAKWPTKLVKNCFLPLREY |
| | | FKYFSTELTSSLGGPSSGAPPPSGGSPAGSPTSTEEGTSESATPE |
| | | SGPGTSTEPSEGSAPGSPAGSPTSTEEGTSTEPSEGSAPGTSTEP |
| | | SELEDKKYSIGLAIGTNSVGWAVITDEYKVPSKKFKVLGNTDRHS |
| | | IKKNLIGALLFDSGETAEATRLKRTARRRYTRRKNRICYLQEIFS |
| | | NEMAKVDDSFFHRLEESFLVEEDKKHERHPIFGNIVDEVAYHEKY |
| | | PTIYHLRKKLVDSTDKADLRLIYLALAHMIKFRGHFLIEGDLNPD |
| | | NSDVDKLFIQLVQTYNQLFEENPINASGVDAKAILSARLSKSRRL |
| | | ENLIAQLPGEKKNGLFGNLIALSLGLTPNFKSNFDLAEDAKLQLS |
| | | KDTYDDDLDNLLAQIGDQYADLFLAAKNLSDAILLSDILRVNTEI |
| | | TKAPLSASMIKRYDEHHQDLILLKALVRQQLPEKYKEIFFDQSKN |
| | | GYAGYIDGGASQEEFYKFIKPILEKMDGTEELLVKLNREDLLRKQ |
| | | RTFDNGSIPHQIHLGELHAILRRQEDFYPFLKDNREKIEKILTFR |
| | | IPYYVGPLARGNSRFAWMTRKSEETITPWNFEEVVDKGASAQSFI |
| | | ERMTNFDKNLPNEKVLPKHSLLYEYFTVYNELTKVKYVTEGMRKP |
| | | AFLSGEQKKAIVDLLFKTNRKVTVKQLKEDYFKKIECFDSVEISG |
| | | VEDRFNASLGTYHDLLKIIKDKDFLDNEENEDILEDIVLTLTLFE |
| | | DREMIEERLKTYAHLFDDKVMKQLKRRRYTGWGRLSRKLINGIRD |
| | | KQSGKTILDFLKSDGFANRNFMQLIHDDSLTFKEDIQKAQVSGQG |
| | | DSLHEHIANLAGSPAIKKGILQTVKVVDELVKVMGRHKPENIVIE |
| | | MARENQTTQKGQKNSRERMKRIEEGIKELGSQILKEHPVENTQLQ |
| | | NEKLYLYYLQNGRDMYVDQELDINRLSDYDVDAIVPQSFLKDDSI |
| | | DNKVLTRSDKNRGKSDNVPSEEVVKKMKNYWRQLLNAKLITQRKF |
| | | DNLTKAERGGLSELDKAGFIKRQLVETRQITKHVAQILDSRMNTK |
| | | YDENDKLIREVKVITLKSKLVSDFRKDFQFYKVREINNYHHAHDA |
| | | YLNAVVGTALIKKYPKLESEFVYGDYKVYDVRKMIAKSEQEIGKA |
| | | TAKYFFYSNIMNFFKTEITLANGEIRKRPLIETNGETGEIVWDKG |
| | | RDFATVRKVLSMPQVNIVKKTEVQTGGFSKESILPKRNSDKLIAR |
| | | KKDWDPKKYGGFDSPTVAYSVLVVAKVEKGKSKKLKSVKELLGIT |
| | | IMERSSFEKNPIDFLEAKGYKEVKKDLIIKLPKYSLFELENGRKR |
| | | MLASAGELQKGNELALPSKYVNFLYLASHYEKLKGSPEDNEQKQL |
| | | FVEQHKHYLDEIIEQISEFSKRVILADANLDKVLSAYNKHRDKPI |
| | | REQAENIIHLFTLINLGAPAAFKYFDTTIDRKRYTSTKEVLDATL |
| | | IHQSITGLYETRIDLSQLGGDSPKKKRKVGVDGSSGSETPGTSES |
| | | ATPESRTLVTFKDVFVDFTREEWKLLDTAQQIVYRNVMLENYKNL |
| | | VSLGYQLTKPDVILRLEKGEEPWLV |
| 10 | CRISPRoff Effector | ATGTACCCATACGATGTTCCAGATTACGCTTCGCCGAAGAAAAAG |
| | nucleotide coding | CGCAAGGTCAATCACGATCAGGAGTTCGACCCCCCTAAGGTGTAC |
| | sequence | CCACCAGTGCCTGCAGAGAAGAGGAAGCCAATCCGGGTGCTGAGC |
| | | CTGTTTGATGGCATCGCCACCGGCCTGCTGGTGCTGAAGGATCTG |
| | | GGCATCCAGGTGGACCGGTACATCGCCTCCGAGGTGTGCGAGGAT |
| | | TCTATCACCGTGGGCATGGTGCGCCACCAGGGCAAGATCATGTAT |
| | | GTGGGCGACGTGCGGTCCGTGACACAGAAGCACATCCAGGAGTGG |
| | | GGCCCATTCGATCTGGTGATCGGCGGCAGCCCCTGTAATGACCTG |
| | | TCCATCGTGAACCCTGCAAGGAAGGGACTGTACGAGGGAACCGGC |
| | | CGGCTGTTCTTTGAGTTTTATAGACTGCTGCACGACGCCAGGCCT |
| | | AAGGAGGGCGACGATAGACCATTCTTTTGGCTGTTCGAGAATGTG |
| | | GTGGCTATGGGCGTGAGCGATAAGAGGGACATCTCCAGGTTTCTG |
| | | GAGTCTAACCCCGTGATGATCGATGCAAAGGAGGTGTCCGCCGCA |
| | | CACAGAGCCAGGTATTTCTGGGGCAATCTGCCAGGAATGAACAGG |
| | | CCACTGGCAAGCACCGTGAATGACAAGCTGGAGCTGCAGGAGTGC |
| | | CTGGAGCACGGAAGGATCGCCAAGTTTTCCAAGGTGCGCACAATC |
| | | ACCACACGGAGCAATTCCATCAAGCAGGGCAAGGATCAGCACTTC |
| | | CCCGTGTTCATGAACGAGAAGGAGGACATCCTGTGGTGTACCGAG |
| | | ATGGAGAGAGTGTTCGGCTTTCCAGTGCACTACACAGACGTGTCT |
| | | AACATGAGCAGGCTGGCAAGGCAGCGGCTGCTGGGCAGATCTTGG |
| | | AGCGTGCCCGTGATCAGGCACCTGTTCGCCCCTCTGAAGGAGTAT |
| | | TTTGCCTGCGTGAGCAGCGGCAACTCCAATGCCAACAGCCGGGGC |
| | | CCCTCTTTCAGCTCCGGATTGGTGCCTCTGAGCCTGAGGGGCTCC |
| | | CACATGGCAGCAATCCCCGCCCTGGACCCCGAGGCCGAGCCTAGC |
| | | ATGGACGTGATCCTGGTGGGCTCTAGCGAGCTGTCCTCTAGCGTG |
| | | TCTCCAGGAACCGGAAGGGATCTGATCGCATACGAGGTGAAGGCC |
| | | AATCAGCGGAACATCGAGGACATCTGTATCTGCTGTGGCAGCCTG |
| | | CAGGTGCACACACAGCACCCACTGTTCGAGGGAGGAATCTGCGCA |
| | | CCCTGTAAGGATAAGTTCCTGGACGCCCTGTTTCTGTACGACGAT |
| | | GACGGCTACCAGTCCTATTGCTCTATCTGCTGTTCCGGCGAGACC |
| | | CTGCTGATCTGCGGCAATCCAGATTGTACAAGGTGCTATTGTTTT |
| | | GAGTGCGTGGACTCTCTGGTGGGACCAGGCACCAGCGGAAAGGTG |
| | | CACGCCATGTCCAACTGGGTGTGCTACCTGTGCCTGCCATCCTCT |
| | | CGCAGCGGACTGCTGCAGCGGAGAAGGAAGTGGAGATCCCAGCTG |
| | | AAGGCCTTCTATGATAGGGAGTCTGAGAACCCCCTGGAGATGTTT |
| | | GAGACCGTGCCAGTGTGGCGCCGGCAGCCCGTGAGGGTGCTGAGC |
| | | CTGTTCGAGGATATCAAGAAGGAGCTGACATCCCTGGGCTTTCTG |
| | | GAGTCCGGCTCTGACCCCGGACAGCTGAAGCACGTGGTGGATGTG |
| | | ACCGACACAGTGCGGAAGGATGTGGAGGAGTGGGGCCCTTTCGAC |
| | | CTGGTGTACGGAGCAACCCCTCCACTGGGACACACATGCGACAGA |
| | | CCCCCTTCTTGGTACCTGTTCCAGTTTCACCGCCTGCTGCAGTAT |
| | | GCAAGGCCAAAGCCAGGCAGCCCTAGACCATTCTTTTGGATGTTC |
| | | GTGGATAATCTGGTGCTGAACAAGGAGGATCTGGACGTGGCCAGC |
| | | AGGTTTCTGGAGATGGAGCCAGTGACCATCCCAGACGTGCACGGC |
| | | GGCTCCCTGCAGAATGCCGTGCGCGTGTGGTCTAACATCCCTGCC |
| | | ATCAGAAGCAGGCACTGGGCACTGGTGAGCGAGGAGGAGCTGTCC |
| | | CTGCTGGCCCAGAATAAGCAGAGCAGCAAGCTGGCCGCCAAGTGG |
| | | CCTACAAAGCTGGTGAAGAACTGCTTCCTGCCACTGCGGGAGTAC |
| | | TTCAAGTATTTTTCCACCGAGCTGACATCTAGCCTGGGAGGACCC |
| | | TCCTCTGGCGCCCCACCACCTAGCGGCGGCTCCCCTGCCGGCTCT |
| | | CCAACCAGCACAGAGGAGGGCACCAGCGAGTCCGCCACACCAGAG |
| | | TCTGGACCTGGCACCAGCACAGAGCCATCCGAGGGCTCTGCCCCA |
| | | GGCTCTCCTGCAGGCAGCCCTACCTCCACCGAAGAGGGCACCAGC |
| | | ACAGAGCCTTCTGAGGGCAGCGCCCCAGGCACCTCTACAGAGCCA |
| | | AGCGAGCTCGAGGACAAGAAGTACAGCATCGGCCTGGCCATCGGC |
| | | ACCAACTCTGTGGGCTGGGCCGTGATCACCGACGAGTACAAGGTG |
| | | CCCAGCAAGAAATTCAAGGTGCTGGGCAACACCGACCGGCACAGC |
| | | ATCAAGAAGAACCTGATCGGAGCCCTGCTGTTCGACAGCGGCGAA |
| | | ACAGCCGAGGCCACCCGGCTGAAGAGAACCGCCAGAAGAAGATAC |
| | | ACCAGACGGAAGAACCGGATCTGCTATCTGCAAGAGATCTTCAGC |
| | | AACGAGATGGCCAAGGTGGACGACAGCTTCTTCCACAGACTGGAA |
| | | GAGTCCTTCCTGGTGGAAGAGGATAAGAAGCACGAGCGGCACCCC |
| | | ATCTTCGGCAACATCGTGGACGAGGTGGCCTACCACGAGAAGTAC |
| | | CCCACCATCTACCACCTGAGAAAGAAACTGGTGGACAGCACCGAC |
| | | AAGGCCGACCTGCGGCTGATCTATCTGGCCCTGGCCCACATGATC |
| | | AAGTTCCGGGGCCACTTCCTGATCGAGGGCGACCTGAACCCCGAC |
| | | AACAGCGACGTGGACAAGCTGTTCATCCAGCTGGTGCAGACCTAC |
| | | AACCAGCTGTTCGAGGAAAACCCCATCAACGCCAGCGGCGTGGAC |
| | | GCCAAGGCCATCCTGTCTGCCAGACTGAGCAAGAGCAGACGGCTG |
| | | GAAAATCTGATCGCCCAGCTGCCCGGCGAGAAGAAGAATGGCCTG |
| | | TTCGGCAACCTGATTGCCCTGAGCCTGGGCCTGACCCCCAACTTC |
| | | AAGAGCAACTTCGACCTGGCCGAGGATGCCAAACTGCAGCTGAGC |
| | | AAGGACACCTACGACGACGACCTGGACAACCTGCTGGCCCAGATC |
| | | GGCGACCAGTACGCCGACCTGTTTCTGGCCGCCAAGAACCTGTCC |
| | | GACGCCATCCTGCTGAGCGACATCCTGAGAGTGAACACCGAGATC |
| | | ACCAAGGCCCCCCTGAGCGCCTCTATGATCAAGAGATACGACGAG |
| | | CACCACCAGGACCTGACCCTGCTGAAAGCTCTCGTGCGGCAGCAG |
| | | CTGCCTGAGAAGTACAAAGAGATTTTCTTCGACCAGAGCAAGAAC |
| | | GGCTACGCCGGCTACATTGACGGCGGAGCCAGCCAGGAAGAGTTC |
| | | TACAAGTTCATCAAGCCCATCCTGGAAAAGATGGACGGCACCGAG |
| | | GAACTGCTCGTGAAGCTGAACAGAGAGGACCTGCTGCGGAAGCAG |
| | | CGGACCTTCGACAACGGCAGCATCCCCCACCAGATCCACCTGGGA |
| | | GAGCTGCACGCCATTCTGCGGCGGCAGGAAGATTTTTACCCATTC |
| | | CTGAAGGACAACCGGGAAAAGATCGAGAAGATCCTGACCTTCCGC |
| | | ATCCCCTACTACGTGGGCCCTCTGGCCAGGGGAAACAGCAGATTC |
| | | GCCTGGATGACCAGAAAGAGCGAGGAAACCATCACCCCCTGGAAC |
| | | TTCGAGGAAGTGGTGGACAAGGGCGCTTCCGCCCAGAGCTTCATC |
| | | GAGCGGATGACCAACTTCGATAAGAACCTGCCCAACGAGAAGGTG |
| | | CTGCCCAAGCACAGCCTGCTGTACGAGTACTTCACCGTGTATAAC |
| | | GAGCTGACCAAAGTGAAATACGTGACCGAGGGAATGAGAAAGCCC |
| | | GCCTTCCTGAGCGGCGAGCAGAAAAAGGCCATCGTGGACCTGCTG |
| | | TTCAAGACCAACCGGAAAGTGACCGTGAAGCAGCTGAAAGAGGAC |
| | | TACTTCAAGAAAATCGAGTGCTTCGACTCCGTGGAAATCTCCGGC |
| | | GTGGAAGATCGGTTCAACGCCTCCCTGGGCACATACCACGATCTG |
| | | CTGAAAATTATCAAGGACAAGGACTTCCTGGACAATGAGGAAAAC |
| | | GAGGACATTCTGGAAGATATCGTGCTGACCCTGACACTGTTTGAG |
| | | GACAGAGAGATGATCGAGGAACGGCTGAAAACCTATGCCCACCTG |
| | | TTCGACGACAAAGTGATGAAGCAGCTGAAGCGGCGGAGATACACC |
| | | GGCTGGGGCAGGCTGAGCCGGAAGCTGATCAACGGCATCCGGGAC |
| | | AAGCAGTCCGGCAAGACAATCCTGGATTTCCTGAAGTCCGACGGC |
| | | TTCGCCAACAGAAACTTCATGCAGCTGATCCACGACGACAGCCTG |
| | | ACCTTTAAAGAGGACATCCAGAAAGCCCAGGTGTCCGGCCAGGGC |
| | | GATAGCCTGCACGAGCACATTGCCAATCTGGCCGGCAGCCCCGCC |
| | | ATTAAGAAGGGCATCCTGCAGACAGTGAAGGTGGTGGACGAGCTC |
| | | GTGAAAGTGATGGGCCGGCACAAGCCCGAGAACATCGTGATCGAA |
| | | ATGGCCAGAGAGAACCAGACCACCCAGAAGGGACAGAAGAACAGC |
| | | CGCGAGAGAATGAAGCGGATCGAAGAGGGCATCAAAGAGCTGGGC |
| | | AGCCAGATCCTGAAAGAACACCCCGTGGAAAACACCCAGCTGCAG |
| | | AACGAGAAGCTGTACCTGTACTACCTGCAGAATGGGCGGGATATG |
| | | TACGTGGACCAGGAACTGGACATCAACCGGCTGTCCGACTACGAT |
| | | GTGGACGCCATCGTGCCTCAGAGCTTTCTGAAGGACGACTCCATC |
| | | GACAACAAGGTGCTGACCAGAAGCGACAAGAACCGGGGCAAGAGC |
| | | GACAACGTGCCCTCCGAAGAGGTCGTGAAGAAGATGAAGAACTAC |
| | | TGGCGGCAGCTGCTGAACGCCAAGCTGATTACCCAGAGAAAGTTC |
| | | GACAATCTGACCAAGGCCGAGAGAGGCGGCCTGAGCGAACTGGAT |
| | | AAGGCCGGCTTCATCAAGAGACAGCTGGTGGAAACCCGGCAGATC |
| | | ACAAAGCACGTGGCACAGATCCTGGACTCCCGGATGAACACTAAG |
| | | TACGACGAGAATGACAAGCTGATCCGGGAAGTGAAAGTGATCACC |
| | | CTGAAGTCCAAGCTGGTGTCCGATTTCCGGAAGGATTTCCAGTTT |
| | | TACAAAGTGCGCGAGATCAACAACTACCACCACGCCCACGACGCC |
| | | TACCTGAACGCCGTCGTGGGAACCGCCCTGATCAAAAAGTACCCT |
| | | AAGCTGGAAAGCGAGTTCGTGTACGGCGACTACAAGGTGTACGAC |
| | | GTGCGGAAGATGATCGCCAAGAGCGAGCAGGAAATCGGCAAGGCT |
| | | ACCGCCAAGTACTTCTTCTACAGCAACATCATGAACTTTTTCAAG |
| | | ACCGAGATTACCCTGGCCAACGGCGAGATCCGGAAGCGGCCTCTG |
| | | ATCGAGACAAACGGCGAAACCGGGGAGATCGTGTGGGATAAGGGC |
| | | CGGGATTTTGCCACCGTGCGGAAAGTGCTGAGCATGCCCCAAGTG |
| | | AATATCGTGAAAAAGACCGAGGTGCAGACAGGCGGCTTCAGCAAA |
| | | GAGTCTATCCTGCCCAAGAGGAACAGCGATAAGCTGATCGCCAGA |
| | | AAGAAGGACTGGGACCCTAAGAAGTACGGCGGCTTCGACAGCCCC |
| | | ACCGTGGCCTATTCTGTGCTGGTGGTGGCCAAAGTGGAAAAGGGC |
| | | AAGTCCAAGAAACTGAAGAGTGTGAAAGAGCTGCTGGGGATCACC |
| | | ATCATGGAAAGAAGCAGCTTCGAGAAGAATCCCATCGACTTTCTG |
| | | GAAGCCAAGGGCTACAAAGAAGTGAAAAAGGACCTGATCATCAAG |
| | | CTGCCTAAGTACTCCCTGTTCGAGCTGGAAAACGGCCGGAAGAGA |
| | | ATGCTGGCCTCTGCCGGCGAACTGCAGAAGGGAAACGAACTGGCC |
| | | CTGCCCTCCAAATATGTGAACTTCCTGTACCTGGCCAGCCACTAT |
| | | GAGAAGCTGAAGGGCTCCCCCGAGGATAATGAGCAGAAACAGCTG |
| | | TTTGTGGAACAGCACAAGCACTACCTGGACGAGATCATCGAGCAG |
| | | ATCAGCGAGTTCTCCAAGAGAGTGATCCTGGCCGACGCTAATCTG |
| | | GACAAAGTGCTGTCCGCCTACAACAAGCACCGGGATAAGCCCATC |
| | | AGAGAGCAGGCCGAGAATATCATCCACCTGTTTACCCTGACCAAT |
| | | CTGGGAGCCCCTGCCGCCTTCAAGTACTTTGACACCACCATCGAC |
| | | CGGAAGAGGTACACCAGCACCAAAGAGGTGCTGGACGCCACCCTG |
| | | ATCCACCAGAGCATCACCGGCCTGTACGAGACACGGATCGACCTG |
| | | TCTCAGCTGGGAGGCGACAGCCCCAAGAAGAAGAGAAAGGTGGGA |
| | | GTCGACGGATCCAGCGGCTCCGAGACCCCAGGCACATCTGAGAGC |
| | | GCCACCCCTGAGTCCCGGACCCTGGTGACATTCAAGGACGTGTTC |
| | | GTGGACTTCACCCGGGAGGAGTGGAAGCTGCTGGACACAGCCCAG |
| | | CAGATCGTGTACAGGAACGTGATGCTGGAGAACTATAAGAATCTG |
| | | GTGTCTCTGGGCTACCAGCTGACAAAGCCAGATGTGATCCTGCGG |
| | | CTGGAGAAGGGAGAGGAGCCCTGGCTGGTG |
| 11 | CRISPRi Effector | MPKKKRKVPKKKRKVYDKKYSIGLAIGTNSVGWAVITDEYKVPSK |
| | amino acid sequence | KFKVLGNTDRHSIKKNLIGALLFDSGETAEATRLKRTARRRYTRR |
| | (2xNLS-dCas9- | KNRICYLQEIFSNEMAKVDDSFFHRLEESFLVEEDKKHERHPIFG |
| | XTEN16- | NIVDEVAYHEKYPTIYHLRKKLVDSTDKADLRLIYLALAHMIKFR |
| | KOX1KRAB- | GHFLIEGDLNPDNSDVDKLFIQLVQTYNQLFEENPINASGVDAKA |
| | 2xNLS) | ILSARLSKSRRLENLIAQLPGEKKNGLFGNLIALSLGLIPNFKSN |
| | | FDLAEDAKLQLSKDTYDDDLDNLLAQIGDQYADLFLAAKNLSDAI |
| | | LLSDILRVNTEITKAPLSASMIKRYDEHHQDLTLLKALVRQQLPE |
| | | KYKEIFFDQSKNGYAGYIDGGASQEEFYKFIKPILEKMDGTEELL |
| | | VKLNREDLLRKQRTFDNGSIPHQIHLGELHAILRRQEDFYPFLKD |
| | | NREKIEKILTFRIPYYVGPLARGNSRFAWMTRKSEETITPWNFEE |
| | | VVDKGASAQSFIERMTNFDKNLPNEKVLPKHSLLYEYFTVYNELT |
| | | KVKYVTEGMRKPAFLSGEQKKAIVDLLFKTNRKVTVKQLKEDYFK |
| | | KIECFDSVEISGVEDRFNASLGTYHDLLKIIKDKDFLDNEENEDI |
| | | LEDIVLTLTLFEDREMIEERLKTYAHLFDDKVMKQLKRRRYTGWG |
| | | RLSRKLINGIRDKQSGKTILDFLKSDGFANRNFMQLIHDDSLTFK |
| | | EDIQKAQVSGQGDSLHEHIANLAGSPAIKKGILQTVKVVDELVKV |
| | | MGRHKPENIVIEMARENQTTQKGQKNSRERMKRIEEGIKELGSQI |
| | | LKEHPVENTQLQNEKLYLYYLQNGRDMYVDQELDINRLSDYDVDA |
| | | IVPQSFLKDDSIDNKVLTRSDKNRGKSDNVPSEEVVKKMKNYWRQ |
| | | LLNAKLITQRKFDNLTKAERGGLSELDKAGFIKRQLVETRQITKH |
| | | VAQILDSRMNTKYDENDKLIREVKVITLKSKLVSDFRKDFQFYKV |
| | | REINNYHHAHDAYLNAVVGTALIKKYPKLESEFVYGDYKVYDVRK |
| | | MIAKSEQEIGKATAKYFFYSNIMNFFKTEITLANGEIRKRPLIET |
| | | NGETGEIVWDKGRDFATVRKVLSMPQVNIVKKTEVQTGGFSKESI |
| | | LPKRNSDKLIARKKDWDPKKYGGFDSPTVAYSVLVVAKVEKGKSK |
| | | KLKSVKELLGITIMERSSFEKNPIDFLEAKGYKEVKKDLIIKLPK |
| | | YSLFELENGRKRMLASAGELQKGNELALPSKYVNFLYLASHYEKL |
| | | KGSPEDNEQKQLFVEQHKHYLDEIIEQISEFSKRVILADANLDKV |
| | | LSAYNKHRDKPIREQAENIIHLFTLTNLGAPAAFKYFDTTIDRKR |
| | | YTSTKEVLDATLIHQSITGLYETRIDLSQLGGDSGSETPGTSESA |
| | | TPESTGRTLVTFKDVFVDFTREEWKLLDTAQQIVYRNVMLENYKN |
| | | LVSLGYQLTKPDVILRLEKGEEPSADYKDDDDKAPKKKRKVPKKK |
| | | RKV |
| 12 | CRISPRi Effector | ATGCCAAAAAAGAAGAGAAAGGTACCGAAGAAAAAAAGAAAGGTA |
| | nucleotide coding | TACGACAAGAAGTACAGCATCGGCCTGGCCATCGGCACCAACTCT |
| | sequence | GTGGGCTGGGCCGTGATCACCGACGAGTACAAGGTGCCCAGCAAG |
| | | AAATTCAAGGTGCTGGGCAACACCGACCGGCACAGCATCAAGAAG |
| | | AACCTGATCGGCGCCCTGCTGTTCGACAGCGGAGAAACAGCCGAG |
| | | GCCACCCGGCTGAAGAGAACCGCCAGAAGAAGATACACCAGACGG |
| | | AAGAACCGGATCTGCTATCTGCAAGAGATCTTCAGCAACGAGATG |
| | | GCCAAGGTGGACGACAGCTTCTTCCACAGACTGGAAGAGTCCTTC |
| | | CTGGTGGAAGAGGATAAGAAGCACGAGCGGCACCCCATCTTCGGC |
| | | AACATCGTGGACGAGGTGGCCTACCACGAGAAGTACCCCACCATC |
| | | TACCACCTGAGAAAGAAACTGGTGGACAGCACCGACAAGGCCGAC |
| | | CTGCGGCTGATCTATCTGGCCCTGGCCCACATGATCAAGTTCCGG |
| | | GGCCACTTCCTGATCGAGGGCGACCTGAACCCCGACAACAGCGAC |
| | | GTGGACAAGCTGTTCATCCAGCTGGTGCAGACCTACAACCAGCTG |
| | | TTCGAGGAAAACCCCATCAACGCCAGCGGCGTGGACGCCAAGGCC |
| | | ATCCTGTCTGCCAGACTGAGCAAGAGCAGACGGCTGGAAAATCTG |
| | | ATCGCCCAGCTGCCCGGCGAGAAGAAGAATGGCCTGTTCGGCAAC |
| | | CTGATTGCCCTGAGCCTGGGCCTGACCCCCAACTTCAAGAGCAAC |
| | | TTCGACCTGGCCGAGGATGCCAAACTGCAGCTGAGCAAGGACACC |
| | | TACGACGACGACCTGGACAACCTGCTGGCCCAGATCGGCGACCAG |
| | | TACGCCGACCTGTTTCTGGCCGCCAAGAACCTGTCCGACGCCATC |
| | | CTGCTGAGCGACATCCTGAGAGTGAACACCGAGATCACCAAGGCC |
| | | CCCCTGAGCGCCTCTATGATCAAGAGATACGACGAGCACCACCAG |
| | | GACCTGACCCTGCTGAAAGCTCTCGTGCGGCAGCAGCTGCCTGAG |
| | | AAGTACAAAGAGATTTTCTTCGACCAGAGCAAGAACGGCTACGCC |
| | | GGCTACATCGATGGCGGAGCCAGCCAGGAAGAGTTCTACAAGTTC |
| | | ATCAAGCCCATCCTGGAAAAGATGGACGGCACCGAGGAACTGCTC |
| | | GTGAAGCTGAACAGAGAGGACCTGCTGCGGAAGCAGCGGACCTTC |
| | | GACAACGGCAGCATCCCCCACCAGATCCACCTGGGAGAGCTGCAC |
| | | GCCATTCTGCGGCGGCAGGAAGATTTTTACCCATTCCTGAAGGAC |
| | | AACCGGGAAAAGATCGAGAAGATCCTGACCTTCCGCATCCCCTAC |
| | | TACGTGGGCCCTCTGGCCAGGGGAAACAGCAGATTCGCCTGGATG |
| | | ACCAGAAAGAGCGAGGAAACCATCACCCCCTGGAACTTCGAGGAA |
| | | GTGGTGGACAAGGGCGCCAGCGCCCAGAGCTTCATCGAGCGGATG |
| | | ACCAACTTCGATAAGAACCTGCCCAACGAGAAGGTGCTGCCCAAG |
| | | CACAGCCTGCTGTACGAGTACTTCACCGTGTACAACGAGCTGACC |
| | | AAAGTGAAATACGTGACCGAGGGAATGAGAAAGCCCGCCTTCCTG |
| | | AGCGGCGAGCAGAAAAAAGCCATCGTGGACCTGCTGTTCAAGACC |
| | | AACCGGAAAGTGACCGTGAAGCAGCTGAAAGAGGACTACTTCAAG |
| | | AAAATCGAGTGCTTCGACTCCGTGGAAATCTCCGGCGTGGAAGAT |
| | | CGGTTCAACGCCTCCCTGGGCACATACCACGATCTGCTGAAAATT |
| | | ATCAAGGACAAGGACTTCCTGGACAATGAGGAAAACGAGGACATT |
| | | CTGGAAGATATCGTGCTGACCCTGACACTGTTTGAGGACAGAGAG |
| | | ATGATCGAGGAACGGCTGAAAACCTATGCCCACCTGTTCGACGAC |
| | | AAAGTGATGAAGCAGCTGAAGCGGCGGAGATACACCGGCTGGGGC |
| | | AGGCTGAGCCGGAAGCTGATCAACGGCATCCGGGACAAGCAGTCC |
| | | GGCAAGACAATCCTGGATTTCCTGAAGTCCGACGGCTTCGCCAAC |
| | | AGAAACTTCATGCAGCTGATCCACGACGACAGCCTGACCTTTAAA |
| | | GAGGACATCCAGAAAGCCCAGGTGTCCGGCCAGGGCGATAGCCTG |
| | | CACGAGCACATTGCCAATCTGGCCGGCAGCCCCGCCATTAAGAAG |
| | | GGCATCCTGCAGACAGTGAAGGTGGTGGACGAGCTCGTGAAAGTG |
| | | ATGGGCCGGCACAAGCCCGAGAACATCGTGATCGAAATGGCCAGA |
| | | GAGAACCAGACCACCCAGAAGGGACAGAAGAACAGCCGCGAGAGA |
| | | ATGAAGCGGATCGAAGAGGGCATCAAAGAGCTGGGCAGCCAGATC |
| | | CTGAAAGAACACCCCGTGGAAAACACCCAGCTGCAGAACGAGAAG |
| | | CTGTACCTGTACTACCTGCAGAATGGGCGGGATATGTACGTGGAC |
| | | CAGGAACTGGACATCAACCGGCTGTCCGACTACGATGTGGACGCT |
| | | ATCGTGCCTCAGAGCTTTCTGAAGGACGACTCCATCGATAACAAA |
| | | GTGCTGACTCGGAGCGACAAGAACCGGGGCAAGAGCGACAACGTG |
| | | CCCTCCGAAGAGGTCGTGAAGAAGATGAAGAACTACTGGCGCCAG |
| | | CTGCTGAATGCCAAGCTGATTACCCAGAGGAAGTTCGACAATCTG |
| | | ACCAAGGCCGAGAGAGGCGGCCTGAGCGAACTGGATAAGGCCGGC |
| | | TTCATCAAGAGACAGCTGGTGGAAACCCGGCAGATCACAAAGCAC |
| | | GTGGCACAGATCCTGGACTCCCGGATGAACACTAAGTACGACGAG |
| | | AACGACAAACTGATCCGGGAAGTGAAAGTGATCACCCTGAAGTCC |
| | | AAGCTGGTGTCCGATTTCCGGAAGGATTTCCAGTTTTACAAAGTG |
| | | CGCGAGATCAACAACTACCACCACGCCCACGACGCCTACCTGAAC |
| | | GCCGTCGTGGGAACCGCCCTGATCAAAAAGTACCCTAAGCTGGAA |
| | | AGCGAGTTCGTGTACGGCGACTACAAGGTGTACGACGTGCGGAAG |
| | | ATGATCGCCAAGAGCGAGCAGGAAATCGGCAAGGCTACCGCCAAG |
| | | TACTTCTTCTACAGCAACATCATGAACTTTTTCAAGACCGAGATT |
| | | ACCCTGGCCAACGGCGAGATCCGGAAGCGGCCTCTGATCGAGACA |
| | | AACGGCGAAACAGGCGAGATCGTGTGGGATAAGGGCCGGGACTTT |
| | | GCCACCGTGCGGAAAGTGCTGTCTATGCCCCAAGTGAATATCGTG |
| | | AAAAAGACCGAGGTGCAGACAGGCGGCTTCAGCAAAGAGTCTATC |
| | | CTGCCCAAGAGGAACAGCGACAAGCTGATCGCCAGAAAGAAGGAC |
| | | TGGGACCCTAAGAAGTACGGCGGCTTCGACAGCCCCACCGTGGCC |
| | | TATTCTGTGCTGGTGGTGGCCAAAGTGGAAAAGGGCAAGTCCAAG |
| | | AAACTGAAGAGTGTGAAAGAGCTGCTGGGGATCACCATCATGGAA |
| | | AGAAGCAGCTTCGAGAAGAATCCCATCGACTTTCTGGAAGCCAAG |
| | | GGCTACAAAGAAGTGAAAAAGGACCTGATCATCAAGCTGCCTAAG |
| | | TACTCCCTGTTCGAGCTGGAAAACGGCCGGAAGAGAATGCTGGCC |
| | | TCTGCCGGCGAACTGCAGAAGGGAAACGAACTGGCCCTGCCCTCC |
| | | AAATATGTGAACTTCCTGTACCTGGCCAGCCACTATGAGAAGCTG |
| | | AAGGGCTCCCCCGAGGATAATGAGCAGAAACAGCTGTTTGTGGAA |
| | | CAGCACAAACACTACCTGGACGAGATCATCGAGCAGATCAGCGAG |
| | | TTCTCCAAGAGAGTGATCCTGGCCGACGCTAATCTGGACAAGGTG |
| | | CTGAGCGCCTACAACAAGCACAGAGACAAGCCTATCAGAGAGCAG |
| | | GCCGAGAATATCATCCACCTGTTTACCCTGACCAATCTGGGAGCC |
| | | CCTGCCGCCTTCAAGTACTTTGACACCACCATCGACCGGAAGAGG |
| | | TACACCAGCACCAAAGAGGTGCTGGACGCCACCCTGATCCACCAG |
| | | AGCATCACCGGCCTGTACGAGACACGGATCGACCTGTCTCAGCTG |
| | | GGAGGCGACAGCGGAAGTGAGACCCCAGGTACATCCGAATCAGCA |
| | | ACGCCTGAAAGCACCGGTCGGACACTGGTGACCTTCAAGGATGTA |
| | | TTTGTGGACTTCACCAGGGAGGAGTGGAAGCTGCTGGACACTGCT |
| | | CAGCAGATCGTGTACAGAAATGTGATGCTGGAGAACTATAAGAAC |
| | | CTGGTTTCCTTGGGTTATCAGCTTACTAAGCCAGATGTGATCCTC |
| | | CGGTTGGAGAAGGGAGAAGAGCCCAGCGCTGATTACAAAGATGAT |
| | | GACGATAAAGCCCCAAAAAAGAAGAGAAAGGTACCGAAGAAAAAA |
| | | AGAAAGGTC |
| 13 | ZFOff 1 (PCSK9- | MPKKKRKVPKKKRKVYNHDQEFDPPKVYPPVPAEKRKPIRVLSLF |
| | ZF-Off) amino acid | DGIATGLLVLKDLGIQVDRYIASEVCEDSITVGMVRHQGKIMYVG |
| | | DVRSVTQKHIQEWGPFDLVIGGSPCNDLSIVNPARKGLYEGTGRL |
| | | FFEFYRLLHDARPKEGDDRPFFWLFENVVAMGVSDKRDISRFLES |
| | | NPVMIDAKEVSAAHRARYFWGNLPGMNRPLASTVNDKLELQECLE |
| | | HGRIAKFSKVRTITTRSNSIKQGKDQHFPVFMNEKEDILWCTEME |
| | | RVFGFPVHYTDVSNMSRLARQRLLGRSWSVPVIRHLFAPLKEYFA |
| | | CVSSGNSNANSRGPSFSSGLVPLSLRGSHMAAIPALDPEAEPSMD |
| | | VILVGSSELSSSVSPGTGRDLIAYEVKANQRNIEDICICCGSLQV |
| | | HTQHPLFEGGICAPCKDKFLDALFLYDDDGYQSYCSICCSGETLL |
| | | ICGNPDCTRCYCFECVDSLVGPGTSGKVHAMSNWVCYLCLPSSRS |
| | | GLLQRRRKWRSQLKAFYDRESENPLEMFETVPVWRRQPVRVLSLF |
| | | EDIKKELTSLGFLESGSDPGQLKHVVDVTDTVRKDVEEWGPFDLV |
| | | YGATPPLGHTCDRPPSWYLFQFHRLLQYARPKPGSPRPFFWMFVD |
| | | NLVLNKEDLDVASRFLEMEPVTIPDVHGGSLQNAVRVWSNIPAIR |
| | | SRHWALVSEEELSLLAQNKQSSKLAAKWPTKLVKNCFLPLREYFK |
| | | YFSTELTSSLGGPSSGAPPPSGGSPAGSPTSTEEGTSESATPESG |
| | | PGTSTEPSEGSAPGSPAGSPISTEEGTSTEPSEGSAPGTSTEPSE |
| | | SRPGERPFQCRICMRNFSTPSKLDRHIRTHTGEKPFQCRICMRNF |
| | | SLAENLRRHLRTHTGSQKPFQCRICMRNFSRQDNLGRHLRTHTGE |
| | | KPFQCRICMRNFSDGGNLGRHLKTHTGGGGSQKPFQCRICMRNFS |
| | | QSPHLKRHLRTHTGEKPFQCRICMRNFSQSTSLQRHLKTHLRGSS |
| | | GVDGSSGSETPGTSESATPESTGDSVAFEDVAVNFTLEEWALLDP |
| | | SQKNLYRDVMRETFRNLASVGKQWEDQNIEDPFKIPRRNISHIPE |
| | | RLCESKEGGQGEESADYKDDDDKAPKKKRKVPKKKRKV |
| 14 | ZFoff nucleotide | ATGCCAAAAAAGAAGAGAAAGGTACCGAAGAAAAAAAGAAAGGTA |
| | coding sequence | TACAATCACGATCAGGAGTTCGACCCCCCTAAGGTGTACCCACCA |
| | | GTGCCTGCAGAGAAGAGGAAGCCAATCCGGGTGCTGAGCCTGTTT |
| | | GATGGCATCGCCACCGGCCTGCTGGTGCTGAAGGATCTGGGCATC |
| | | CAGGTGGACCGGTACATCGCCTCCGAGGTGTGCGAGGATTCTATC |
| | | ACCGTGGGCATGGTGCGCCACCAGGGCAAGATCATGTATGTGGGC |
| | | GACGTGCGGTCCGTGACACAGAAGCACATCCAGGAGTGGGGCCCA |
| | | TTCGATCTGGTGATCGGCGGCAGCCCCTGTAATGACCTGTCCATC |
| | | GTGAACCCTGCAAGGAAGGGACTGTACGAGGGAACCGGCCGGCTG |
| | | TTCTTTGAGTTTTATAGACTGCTGCACGACGCCAGGCCTAAGGAG |
| | | GGCGACGATAGACCATTCTTTTGGCTGTTCGAGAATGTGGTGGCT |
| | | ATGGGCGTGAGCGATAAGAGGGACATCTCCAGGTTTCTGGAGTCT |
| | | AACCCCGTGATGATCGATGCAAAGGAGGTGTCCGCCGCACACAGA |
| | | GCCAGGTATTTCTGGGGCAATCTGCCAGGAATGAACAGGCCACTG |
| | | GCAAGCACCGTGAATGACAAGCTGGAGCTGCAGGAGTGCCTGGAG |
| | | CACGGAAGGATCGCCAAGTTTTCCAAGGTGCGCACAATCACCACA |
| | | CGGAGCAATTCCATCAAGCAGGGCAAGGATCAGCACTTCCCCGTG |
| | | TTCATGAACGAGAAGGAGGACATCCTGTGGTGTACCGAGATGGAG |
| | | AGAGTGTTCGGCTTTCCAGTGCACTACACAGACGTGTCTAACATG |
| | | AGCAGGCTGGCAAGGCAGCGGCTGCTGGGCAGATCTTGGAGCGTG |
| | | CCCGTGATCAGGCACCTGTTCGCCCCTCTGAAGGAGTATTTTGCC |
| | | TGCGTGAGCAGCGGCAACTCCAATGCCAACAGCCGGGGCCCCTCT |
| | | TTCAGCTCCGGATTGGTGCCTCTGAGCCTGAGGGGCTCCCACATG |
| | | GCAGCAATCCCCGCCCTGGACCCCGAGGCCGAGCCTAGCATGGAC |
| | | GTGATCCTGGTGGGCTCTAGCGAGCTGTCCTCTAGCGTGTCTCCA |
| | | GGAACCGGAAGGGATCTGATCGCATACGAGGTGAAGGCCAATCAG |
| | | CGGAACATCGAGGACATCTGTATCTGCTGTGGCAGCCTGCAGGTG |
| | | CACACACAGCACCCACTGTTCGAGGGAGGAATCTGCGCACCCTGT |
| | | AAGGATAAGTTCCTGGACGCCCTGTTTCTGTACGACGATGACGGC |
| | | TACCAGTCCTATTGCTCTATCTGCTGTTCCGGCGAGACCCTGCTG |
| | | ATCTGCGGCAATCCAGATTGTACAAGGTGCTATTGTTTTGAGTGC |
| | | GTGGACTCTCTGGTGGGACCAGGCACCAGCGGAAAGGTGCACGCC |
| | | ATGTCCAACTGGGTGTGCTACCTGTGCCTGCCATCCTCTCGCAGC |
| | | GGACTGCTGCAGCGGAGAAGGAAGTGGAGATCCCAGCTGAAGGCC |
| | | TTCTATGATAGGGAGTCTGAGAACCCCCTGGAGATGTTTGAGACC |
| | | GTGCCAGTGTGGCGCCGGCAGCCCGTGAGGGTGCTGAGCCTGTTC |
| | | GAGGATATCAAGAAGGAGCTGACATCCCTGGGCTTTCTGGAGTCC |
| | | GGCTCTGACCCCGGACAGCTGAAGCACGTGGTGGATGTGACCGAC |
| | | ACAGTGCGGAAGGATGTGGAGGAGTGGGGCCCTTTCGACCTGGTG |
| | | TACGGAGCAACCCCTCCACTGGGACACACATGCGACAGACCCCCT |
| | | TCTTGGTACCTGTTCCAGTTTCACCGCCTGCTGCAGTATGCAAGG |
| | | CCAAAGCCAGGCAGCCCTAGACCATTCTTTTGGATGTTCGTGGAT |
| | | AATCTGGTGCTGAACAAGGAGGATCTGGACGTGGCCAGCAGGTTT |
| | | CTGGAGATGGAGCCAGTGACCATCCCAGACGTGCACGGCGGCTCC |
| | | CTGCAGAATGCCGTGCGCGTGTGGTCTAACATCCCTGCCATCAGA |
| | | AGCAGGCACTGGGCACTGGTGAGCGAGGAGGAGCTGTCCCTGCTG |
| | | GCCCAGAATAAGCAGAGCAGCAAGCTGGCCGCCAAGTGGCCTACA |
| | | AAGCTGGTGAAGAACTGCTTCCTGCCACTGCGGGAGTACTTCAAG |
| | | TATTTTTCCACCGAGCTGACATCTAGCCTGGGAGGACCCTCCTCT |
| | | GGCGCCCCACCACCTAGCGGCGGCTCCCCTGCCGGCTCTCCAACC |
| | | AGCACAGAGGAGGGCACCAGCGAGTCCGCCACACCAGAGTCTGGA |
| | | CCTGGCACCAGCACAGAGCCATCCGAGGGCTCTGCCCCAGGCTCT |
| | | CCTGCAGGCAGCCCTACCTCCACCGAAGAGGGCACCAGCACAGAG |
| | | CCTTCTGAGGGCAGCGCCCCAGGCACCTCTACAGAGCCAAGCGAG |
| | | TCCCGGCCAGGGGAACGGCCCTTCCAGTGTCGGATCTGCATGAGA |
| | | AACTTTTCAACACCCTCAAAGCTGGATAGGCATACACGCACCCAC |
| | | ACTGGAGAGAAACCCTTTCAGTGCAGGATATGTATGCGGAATTTT |
| | | TCCCTGGCTGAGAACCTCCGAAGGCACCTGAGAACACATACCGGG |
| | | AGTCAGAAGCCTTTCCAATGCCGGATTTGCATGAGGAACTTCTCC |
| | | CGCCAAGATAACCTGGGCCGCCACCTGCGCACACATACAGGCGAG |
| | | AAGCCATTCCAGTGTAGGATCTGCATGCGCAATTTTAGCGACGGC |
| | | GGTAACTTGGGACGCCACCTCAAAACGCATACAGGTGGCGGTGGC |
| | | TCCCAGAAGCCCTTTCAGTGCAGGATCTGCATGAGGAATTTTAGT |
| | | CAGTCCCCACATCTTAAAAGACACTTGCGGACGCATACTGGAGAG |
| | | AAGCCCTTTCAGTGTAGGATTTGTATGCGGAACTTCAGCCAGAGC |
| | | ACAAGCCTGCAACGCCATCTCAAAACTCATTTGCGCGGGTCTAGC |
| | | GGAGTCGACGGATCCAGCGGCTCCGAGACCCCAGGCACATCTGAG |
| | | AGCGCCACCCCTGAGTCCACCGGTGACTCCGTTGCTTTCGAGGAC |
| | | GTGGCCGTGAACTTCACACTTGAGGAATGGGCCTTGCTCGACCCA |
| | | AGTCAGAAGAATCTGTACAGAGACGTGATGCGGGAGACATTCAGG |
| | | AATCTCGCCAGTGTCGGAAAGCAGTGGGAAGACCAGAACATCGAA |
| | | GATCCTTTCAAGATACCACGGCGCAATATCTCCCACATTCCTGAG |
| | | AGGCTGTGTGAATCTAAGGAAGGCGGACAAGGTGAGGAAAGCGCT |
| | | GATTACAAAGATGATGACGATAAAGCCCCCAAGAAGAAAAGGAAG |
| | | GTCCCAAAGAAAAAAAGAAAGGTG |
| 15 | ZFOff 2 (CLTA-ZF- | MPKKKRKVPKKKRKVYNHDQEFDPPKVYPPVPAEKRKPIRVLSLF |
| | Off) amino acid | DGIATGLLVLKDLGIQVDRYIASEVCEDSITVGMVRHQGKIMYVG |
| | | DVRSVTQKHIQEWGPFDLVIGGSPCNDLSIVNPARKGLYEGTGRL |
| | | FFEFYRLLHDARPKEGDDRPFFWLFENVVAMGVSDKRDISRFLES |
| | | NPVMIDAKEVSAAHRARYFWGNLPGMNRPLASTVNDKLELQECLE |
| | | HGRIAKFSKVRTITTRSNSIKQGKDQHFPVFMNEKEDILWCTEME |
| | | RVFGFPVHYTDVSNMSRLARQRLLGRSWSVPVIRHLFAPLKEYFA |
| | | CVSSGNSNANSRGPSFSSGLVPLSLRGSHMAAIPALDPEAEPSMD |
| | | VILVGSSELSSSVSPGTGRDLIAYEVKANQRNIEDICICCGSLQV |
| | | HTQHPLFEGGICAPCKDKFLDALFLYDDDGYQSYCSICCSGETLL |
| | | ICGNPDCTRCYCFECVDSLVGPGTSGKVHAMSNWVCYLCLPSSRS |
| | | GLLQRRRKWRSQLKAFYDRESENPLEMFETVPVWRRQPVRVLSLF |
| | | EDIKKELTSLGFLESGSDPGQLKHVVDVTDTVRKDVEEWGPFDLV |
| | | YGATPPLGHTCDRPPSWYLFQFHRLLQYARPKPGSPRPFFWMFVD |
| | | NLVLNKEDLDVASRFLEMEPVTIPDVHGGSLQNAVRVWSNIPAIR |
| | | SRHWALVSEEELSLLAQNKQSSKLAAKWPTKLVKNCFLPLREYFK |
| | | YFSTELTSSLGGPSSGAPPPSGGSPAGSPTSTEEGTSESATPESG |
| | | PGTSTEPSEGSAPGSPAGSPTSTEEGTSTEPSEGSAPGTSTEPSE |
| | | ERPFQCRICMRNFSHKQHRDAHIRTHTGEKPFACDICGRKFARSA |
| | | NLTRHTKIHTGSQKPFQCRICMRNFSRSDNLSEHIRTHTGEKPFA |
| | | CDICGRKFATSANLSRHTKIHTGSQKPFQCRICMRNFSIRSTLRD |
| | | HIRTHTGEKPFACDICGRKFARTPVRMGHTKIHLRQKDAARGSSG |
| | | VDGSSGSETPGTSESATPESTGDSVAFEDVAVNFTLEEWALLDPS |
| | | QKNLYRDVMRETFRNLASVGKQWEDQNIEDPFKIPRRNISHIPER |
| | | LCESKEGGQGEESADYKDDDDKAPKKKRKVPKKKRKV |
| 16 | ZFOff 2 nucleotide | ATGCCAAAAAAGAAGAGAAAGGTACCGAAGAAAAAAAGAAAGGTA |
| | coding sequence | TACAATCACGATCAGGAGTTCGACCCCCCTAAGGTGTACCCACCA |
| | | GTGCCTGCAGAGAAGAGGAAGCCAATCCGGGTGCTGAGCCTGTTT |
| | | GATGGCATCGCCACCGGCCTGCTGGTGCTGAAGGATCTGGGCATC |
| | | CAGGTGGACCGGTACATCGCCTCCGAGGTGTGCGAGGATTCTATC |
| | | ACCGTGGGCATGGTGCGCCACCAGGGCAAGATCATGTATGTGGGC |
| | | GACGTGCGGTCCGTGACACAGAAGCACATCCAGGAGTGGGGCCCA |
| | | TTCGATCTGGTGATCGGCGGCAGCCCCTGTAATGACCTGTCCATC |
| | | GTGAACCCTGCAAGGAAGGGACTGTACGAGGGAACCGGCCGGCTG |
| | | TTCTTTGAGTTTTATAGACTGCTGCACGACGCCAGGCCTAAGGAG |
| | | GGCGACGATAGACCATTCTTTTGGCTGTTCGAGAATGTGGTGGCT |
| | | ATGGGCGTGAGCGATAAGAGGGACATCTCCAGGTTTCTGGAGTCT |
| | | AACCCCGTGATGATCGATGCAAAGGAGGTGTCCGCCGCACACAGA |
| | | GCCAGGTATTTCTGGGGCAATCTGCCAGGAATGAACAGGCCACTG |
| | | GCAAGCACCGTGAATGACAAGCTGGAGCTGCAGGAGTGCCTGGAG |
| | | CACGGAAGGATCGCCAAGTTTTCCAAGGTGCGCACAATCACCACA |
| | | CGGAGCAATTCCATCAAGCAGGGCAAGGATCAGCACTTCCCCGTG |
| | | TTCATGAACGAGAAGGAGGACATCCTGTGGTGTACCGAGATGGAG |
| | | AGAGTGTTCGGCTTTCCAGTGCACTACACAGACGTGTCTAACATG |
| | | AGCAGGCTGGCAAGGCAGCGGCTGCTGGGCAGATCTTGGAGCGTG |
| | | CCCGTGATCAGGCACCTGTTCGCCCCTCTGAAGGAGTATTTTGCC |
| | | TGCGTGAGCAGCGGCAACTCCAATGCCAACAGCCGGGGCCCCTCT |
| | | TTCAGCTCCGGATTGGTGCCTCTGAGCCTGAGGGGCTCCCACATG |
| | | GCAGCAATCCCCGCCCTGGACCCCGAGGCCGAGCCTAGCATGGAC |
| | | GTGATCCTGGTGGGCTCTAGCGAGCTGTCCTCTAGCGTGTCTCCA |
| | | GGAACCGGAAGGGATCTGATCGCATACGAGGTGAAGGCCAATCAG |
| | | CGGAACATCGAGGACATCTGTATCTGCTGTGGCAGCCTGCAGGTG |
| | | CACACACAGCACCCACTGTTCGAGGGAGGAATCTGCGCACCCTGT |
| | | AAGGATAAGTTCCTGGACGCCCTGTTTCTGTACGACGATGACGGC |
| | | TACCAGTCCTATTGCTCTATCTGCTGTTCCGGCGAGACCCTGCTG |
| | | ATCTGCGGCAATCCAGATTGTACAAGGTGCTATTGTTTTGAGTGC |
| | | GTGGACTCTCTGGTGGGACCAGGCACCAGCGGAAAGGTGCACGCC |
| | | ATGTCCAACTGGGTGTGCTACCTGTGCCTGCCATCCTCTCGCAGC |
| | | GGACTGCTGCAGCGGAGAAGGAAGTGGAGATCCCAGCTGAAGGCC |
| | | TTCTATGATAGGGAGTCTGAGAACCCCCTGGAGATGTTTGAGACC |
| | | GTGCCAGTGTGGCGCCGGCAGCCCGTGAGGGTGCTGAGCCTGTTC |
| | | GAGGATATCAAGAAGGAGCTGACATCCCTGGGCTTTCTGGAGTCC |
| | | GGCTCTGACCCCGGACAGCTGAAGCACGTGGTGGATGTGACCGAC |
| | | ACAGTGCGGAAGGATGTGGAGGAGTGGGGCCCTTTCGACCTGGTG |
| | | TACGGAGCAACCCCTCCACTGGGACACACATGCGACAGACCCCCT |
| | | TCTTGGTACCTGTTCCAGTTTCACCGCCTGCTGCAGTATGCAAGG |
| | | CCAAAGCCAGGCAGCCCTAGACCATTCTTTTGGATGTTCGTGGAT |
| | | AATCTGGTGCTGAACAAGGAGGATCTGGACGTGGCCAGCAGGTTT |
| | | CTGGAGATGGAGCCAGTGACCATCCCAGACGTGCACGGCGGCTCC |
| | | CTGCAGAATGCCGTGCGCGTGTGGTCTAACATCCCTGCCATCAGA |
| | | AGCAGGCACTGGGCACTGGTGAGCGAGGAGGAGCTGTCCCTGCTG |
| | | GCCCAGAATAAGCAGAGCAGCAAGCTGGCCGCCAAGTGGCCTACA |
| | | AAGCTGGTGAAGAACTGCTTCCTGCCACTGCGGGAGTACTTCAAG |
| | | TATTTTTCCACCGAGCTGACATCTAGCCTGGGAGGACCCTCCTCT |
| | | GGCGCCCCACCACCTAGCGGCGGCTCCCCTGCCGGCTCTCCAACC |
| | | AGCACAGAGGAGGGCACCAGCGAGTCCGCCACACCAGAGTCTGGA |
| | | CCTGGCACCAGCACAGAGCCATCCGAGGGCTCTGCCCCAGGCTCT |
| | | CCTGCAGGCAGCCCTACCTCCACCGAAGAGGGCACCAGCACAGAG |
| | | CCTTCTGAGGGCAGCGCCCCAGGCACCTCTACAGAGCCAAGCGAG |
| | | GAGAGACCTTTTCAGTGCCGGATTTGTATGCGCAATTTCTCCCAC |
| | | AAGCAGCACCGCGACGCCCATATTAGAACACATACTGGAGAAAAG |
| | | CCCTTCGCTTGCGACATCTGTGGACGGAAGTTCGCCCGCTCCGCC |
| | | AACCTGACCCGCCACACTAAAATCCATACCGGCAGCCAAAAGCCA |
| | | TTCCAATGTCGCATCTGTATGCGGAACTTTAGCCGCTCCGACAAC |
| | | CTGTCCGAGCATATTCGGACTCACACAGGGGAGAAACCATTTGCA |
| | | TGTGATATCTGCGGCAGAAAATTCGCTACCTCCGCCAACCTGTCC |
| | | CGCCATACCAAGATACACACGGGATCTCAGAAGCCCTTCCAGTGT |
| | | CGAATCTGCATGCGTAACTTCAGTATCCGCTCCACCCTGCGCGAC |
| | | CACATCCGCACCCACACCGGCGAGAAGCCTTTTGCCTGTGACATT |
| | | TGTGGGAGGAAATTTGCCCGCACCCCGGTGCGCATGGGCCATACC |
| | | AAGATACACCTGCGCCAAAAAGATGCGGCCCGGGGATCTAGCGGA |
| | | GTCGACGGATCCAGCGGCTCCGAGACCCCAGGCACATCTGAGAGC |
| | | GCCACCCCTGAGTCCACCGGTGACTCCGTTGCTTTCGAGGACGTG |
| | | GCCGTGAACTTCACACTTGAGGAATGGGCCTTGCTCGACCCAAGT |
| | | CAGAAGAATCTGTACAGAGACGTGATGCGGGAGACATTCAGGAAT |
| | | CTCGCCAGTGTCGGAAAGCAGTGGGAAGACCAGAACATCGAAGAT |
| | | CCTTTCAAGATACCACGGCGCAATATCTCCCACATTCCTGAGAGG |
| | | CTGTGTGAATCTAAGGAAGGCGGACAAGGTGAGGAAAGCGCTGAT |
| | | TACAAAGATGATGACGATAAAGCCCCCAAGAAGAAAAGGAAGGTC |
| | | CCAAAGAAAAAAAGAAAGGTGTGAa |
| 24 | Full Nucleic Acid | GACATTGATTATTGACTAGTTATTAATAGTAATCAATTACGGGGT |
| | Sequence of | CATTAGTTCATAGCCCATATATGGAGTTCCGCGTTACATAACTTA |
| | Adjacent Zinc Hand | CGGTAAATGGCCCGCCTGGCTGACCGCCCAACGACCCCCGCCCAT |
| | PCSK9-CLTA | TGACGTCAATAATGACGTATGTTCCCATAGTAACGCCAATAGGGA |
| | | CTTTCCATTGACGTCAATGGGTGGAGTATTTACGGTAAACTGCCC |
| | | ACTTGGCAGTACATCAAGTGTATCATATGCCAAGTACGCCCCCTA |
| | | TTGACGTCAATGACGGTAAATGGCCCGCCTGGCATTATGCCCAGT |
| | | ACATGACCTTATGGGACTTTCCTACTTGGCAGTACATCTACGTAT |
| | | TAGTCATCGCTATTACCATGGTGATGCGGTTTTGGCAGTACATCA |
| | | ATGGGCGTGGATAGCGGTTTGACTCACGGGGATTTCCAAGTCTCC |
| | | ACCCCATTGACGTCAATGGGAGTTTGTTTTGGCACCAAAATCAAC |
| | | GGGACTTTCCAAAATGTCGTAACAACTCCGCCCCATTGACGCAAA |
| | | TGGGCGGTAGGCGTGTACGGTGGGAGGTCTATATAAGCAGAGCTC |
| | | TCTGGCTAACTAGAGAACCCACTGCTTACTGGCTTATCGAAATTA |
| | | ATACGACTCACTATAAGGGGGCGCTCGAGCAGGTTCAGAAGGAGA |
| | | TCAAAAACCCCCAAGGATCAAACATGCCAAAAAAGAAGAGAAAGG |
| | | TACCGAAGAAAAAAAGAAAGGTATACAATCACGATCAGGAGTTCG |
| | | ACCCCCCTAAGGTGTACCCACCAGTGCCTGCAGAGAAGAGGAAGC |
| | | CAATCCGGGTGCTGAGCCTGTTTGATGGCATCGCCACCGGCCTGC |
| | | TGGTGCTGAAGGATCTGGGCATCCAGGTGGACCGGTACATCGCCT |
| | | CCGAGGTGTGCGAGGATTCTATCACCGTGGGCATGGTGCGCCACC |
| | | AGGGCAAGATCATGTATGTGGGCGACGTGCGGTCCGTGACACAGA |
| | | AGCACATCCAGGAGTGGGGCCCATTCGATCTGGTGATCGGCGGCA |
| | | GCCCCTGTAATGACCTGTCCATCGTGAACCCTGCAAGGAAGGGAC |
| | | TGTACGAGGGAACCGGCCGGCTGTTCTTTGAGTTTTATAGACTGC |
| | | TGCACGACGCCAGGCCTAAGGAGGGCGACGATAGACCATTCTTTT |
| | | GGCTGTTCGAGAATGTGGTGGCTATGGGCGTGAGCGATAAGAGGG |
| | | ACATCTCCAGGTTTCTGGAGTCTAACCCCGTGATGATCGATGCAA |
| | | AGGAGGTGTCCGCCGCACACAGAGCCAGGTATTTCTGGGGCAATC |
| | | TGCCAGGAATGAACAGGCCACTGGCAAGCACCGTGAATGACAAGC |
| | | TGGAGCTGCAGGAGTGCCTGGAGCACGGAAGGATCGCCAAGTTTT |
| | | CCAAGGTGCGCACAATCACCACACGGAGCAATTCCATCAAGCAGG |
| | | GCAAGGATCAGCACTTCCCCGTGTTCATGAACGAGAAGGAGGACA |
| | | TCCTGTGGTGTACCGAGATGGAGAGAGTGTTCGGCTTTCCAGTGC |
| | | ACTACACAGACGTGTCTAACATGAGCAGGCTGGCAAGGCAGCGGC |
| | | TGCTGGGCAGATCTTGGAGCGTGCCCGTGATCAGGCACCTGTTCG |
| | | CCCCTCTGAAGGAGTATTTTGCCTGCGTGAGCAGCGGCAACTCCA |
| | | ATGCCAACAGCCGGGGCCCCTCTTTCAGCTCCGGATTGGTGCCTC |
| | | TGAGCCTGAGGGGCTCCCACATGGCAGCAATCCCCGCCCTGGACC |
| | | CCGAGGCCGAGCCTAGCATGGACGTGATCCTGGTGGGCTCTAGCG |
| | | AGCTGTCCTCTAGCGTGTCTCCAGGAACCGGAAGGGATCTGATCG |
| | | CATACGAGGTGAAGGCCAATCAGCGGAACATCGAGGACATCTGTA |
| | | TCTGCTGTGGCAGCCTGCAGGTGCACACACAGCACCCACTGTTCG |
| | | AGGGAGGAATCTGCGCACCCTGTAAGGATAAGTTCCTGGACGCCC |
| | | TGTTTCTGTACGACGATGACGGCTACCAGTCCTATTGCTCTATCT |
| | | GCTGTTCCGGCGAGACCCTGCTGATCTGCGGCAATCCAGATTGTA |
| | | CAAGGTGCTATTGTTTTGAGTGCGTGGACTCTCTGGTGGGACCAG |
| | | GCACCAGCGGAAAGGTGCACGCCATGTCCAACTGGGTGTGCTACC |
| | | TGTGCCTGCCATCCTCTCGCAGCGGACTGCTGCAGCGGAGAAGGA |
| | | AGTGGAGATCCCAGCTGAAGGCCTTCTATGATAGGGAGTCTGAGA |
| | | ACCCCCTGGAGATGTTTGAGACCGTGCCAGTGTGGCGCCGGCAGC |
| | | CCGTGAGGGTGCTGAGCCTGTTCGAGGATATCAAGAAGGAGCTGA |
| | | CATCCCTGGGCTTTCTGGAGTCCGGCTCTGACCCCGGACAGCTGA |
| | | AGCACGTGGTGGATGTGACCGACACAGTGCGGAAGGATGTGGAGG |
| | | AGTGGGGCCCTTTCGACCTGGTGTACGGAGCAACCCCTCCACTGG |
| | | GACACACATGCGACAGACCCCCTTCTTGGTACCTGTTCCAGTTTC |
| | | ACCGCCTGCTGCAGTATGCAAGGCCAAAGCCAGGCAGCCCTAGAC |
| | | CATTCTTTTGGATGTTCGTGGATAATCTGGTGCTGAACAAGGAGG |
| | | ATCTGGACGTGGCCAGCAGGTTTCTGGAGATGGAGCCAGTGACCA |
| | | TCCCAGACGTGCACGGCGGCTCCCTGCAGAATGCCGTGCGCGTGT |
| | | GGTCTAACATCCCTGCCATCAGAAGCAGGCACTGGGCACTGGTGA |
| | | GCGAGGAGGAGCTGTCCCTGCTGGCCCAGAATAAGCAGAGCAGCA |
| | | AGCTGGCCGCCAAGTGGCCTACAAAGCTGGTGAAGAACTGCTTCC |
| | | TGCCACTGCGGGAGTACTTCAAGTATTTTTCCACCGAGCTGACAT |
| | | CTAGCCTGGGAGGACCCTCCTCTGGCGCCCCACCACCTAGCGGCG |
| | | GCTCCCCTGCCGGCTCTCCAACCAGCACAGAGGAGGGCACCAGCG |
| | | AGTCCGCCACACCAGAGTCTGGACCTGGCACCAGCACAGAGCCAT |
| | | CCGAGGGCTCTGCCCCAGGCTCTCCTGCAGGCAGCCCTACCTCCA |
| | | CCGAAGAGGGCACCAGCACAGAGCCTTCTGAGGGCAGCGCCCCAG |
| | | GCACCTCTACAGAGCCAAGCGAGTCCCGGCCAGGGGAACGGCCCT |
| | | TCCAGTGTCGGATCTGCATGAGAAACTTTTCAACACCCTCAAAGC |
| | | TGGATAGGCATACACGCACCCACACTGGAGAGAAACCCTTTCAGT |
| | | GCAGGATATGTATGCGGAATTTTTCCCTGGCTGAGAACCTCCGAA |
| | | GGCACCTGAGAACACATACCGGGAGTCAGAAGCCTTTCCAATGCC |
| | | GGATTTGCATGAGGAACTTCTCCCGCCAAGATAACCTGGGCCGCC |
| | | ACCTGCGCACACATACAGGCGAGAAGCCATTCCAGTGTAGGATCT |
| | | GCATGCGCAATTTTAGCGACGGCGGTAACTTGGGACGCCACCTCA |
| | | AAACGCATACAGGTGGCGGTGGCTCCCAGAAGCCCTTTCAGTGCA |
| | | GGATCTGCATGAGGAATTTTAGTCAGTCCCCACATCTTAAAAGAC |
| | | ACTTGCGGACGCATACTGGAGAGAAGCCCTTTCAGTGTAGGATTT |
| | | GTATGCGGAACTTCAGCCAGAGCACAAGCCTGCAACGCCATCTCA |
| | | AAACTCATTTGCGCGGGTCTGGTGTTGATGGCTCCTCTGGTTCTG |
| | | AAACTCCTGGTACTTCTGAGTCTGCTACTCCCGAATCTGAGAGAC |
| | | CTTTTCAGTGCCGGATTTGTATGCGCAATTTCTCCCACAAGCAGC |
| | | ACCGCGACGCCCATATTAGAACACATACTGGAGAAAAGCCCTTCG |
| | | CTTGCGACATCTGTGGACGGAAGTTCGCCCGCTCCGCCAACCTGA |
| | | CCCGCCACACTAAAATCCATACCGGCAGCCAAAAGCCATTCCAAT |
| | | GTCGCATCTGTATGCGGAACTTTAGCCGCTCCGACAACCTGTCCG |
| | | AGCATATTCGGACTCACACAGGGGAGAAACCATTTGCATGTGATA |
| | | TCTGCGGCAGAAAATTCGCTACCTCCGCCAACCTGTCCCGCCATA |
| | | CCAAGATACACACGGGATCTCAGAAGCCCTTCCAGTGTCGAATCT |
| | | GCATGCGTAACTTCAGTATCCGCTCCACCCTGCGCGACCACATCC |
| | | GCACCCACACCGGCGAGAAGCCTTTTGCCTGTGACATTTGTGGGA |
| | | GGAAATTTGCCCGCACCCCGGTGCGCATGGGCCATACCAAGATAC |
| | | ACCTGCGCCAAAAAGATGCGGCCCGGGGATCTAGCCCCAAGAAGA |
| | | AGAGAAAGGTGGGAGTCGACGGATCCAGCGGCTCCGAGACCCCAG |
| | | GCACATCTGAGAGCGCCACCCCTGAGTCCACCGGTGACTCCGTTG |
| | | CTTTCGAGGACGTGGCCGTGAACTTCACACTTGAGGAATGGGCCT |
| | | TGCTCGACCCAAGTCAGAAGAATCTGTACAGAGACGTGATGCGGG |
| | | AGACATTCAGGAATCTCGCCAGTGTCGGAAAGCAGTGGGAAGACC |
| | | AGAACATCGAAGATCCTTTCAAGATACCACGGCGCAATATCTCCC |
| | | ACATTCCTGAGAGGCTGTGTGAATCTAAGGAAGGCGGACAAGGTG |
| | | AGGAAAGCGCTGATTACAAAGATGATGACGATAAAGCCCCCAAGA |
| | | AGAAAAGGAAGGTCCCAAAGAAAAAAAGAAAGGTGTGAGGATCCT |
| | | GAGTCTAGAAATCAACCTCTGGATTACAAAATTTGTGAAAGATTG |
| | | ACTGGTATTCTTAACTATGTTGCTCCTTTTACGCTATGTGGATAC |
| | | GCTGCTTTAATGCCTTTGTATCATGCTATTGCTTCCCGTATGGCT |
| | | TTCATTTTCTCCTCCTTGTATAAATCCTGGTTGCTGTCTCTTTAT |
| | | GAGGAGTTGTGGCCCGTTGTCAGGCAACGTGGCGTGGTGTGCACT |
| | | GTGTTTGCTGACGCAACCCCCACTGGTTGGGGCATTGCCACCACC |
| | | TGTCAGCTCCTTTCCGGGACTTTCGCTTTCCCCCTCCCTATTGCC |
| | | ACGGCGGAACTCATCGCCGCCTGCCTTGCCCGCTGCTGGACAGGG |
| | | GCTCGGCTGTTGGGCACTGACAATTCCGTGGTGTTGTCGGGGAAA |
| | | TCATCGTCCTTTCCTTGGCTGCTCGCCTGTGTTGCCACCTGGATT |
| | | CTGCGCGGGACGTCCTTCTGCTACGTCCCTTCGGCCCTCAATCCA |
| | | GCGGACCTTCCTTCCCGCGGCCTGCTGCCGGCTCTGCGGCCTCTT |
| | | CCGCGTCTTCGCCTTCGCCCTCAGACGAGTCGGATCTCCCTTTGG |
| | | GCCGCCTCCCCGCCTGTTAATT |
| 25 | Full Nucleic Acid | GACATTGATTATTGACTAGTTATTAATAGTAATCAATTACGGGGT |
| | Sequence of | CATTAGTTCATAGCCCATATATGGAGTTCCGCGTTACATAACTTA |
| | Adjacent Zinc Hand | CGGTAAATGGCCCGCCTGGCTGACCGCCCAACGACCCCCGCCCAT |
| | CLTA-PCSK9 | TGACGTCAATAATGACGTATGTTCCCATAGTAACGCCAATAGGGA |
| | | CTTTCCATTGACGTCAATGGGTGGAGTATTTACGGTAAACTGCCC |
| | | ACTTGGCAGTACATCAAGTGTATCATATGCCAAGTACGCCCCCTA |
| | | TTGACGTCAATGACGGTAAATGGCCCGCCTGGCATTATGCCCAGT |
| | | ACATGACCTTATGGGACTTTCCTACTTGGCAGTACATCTACGTAT |
| | | TAGTCATCGCTATTACCATGGTGATGCGGTTTTGGCAGTACATCA |
| | | ATGGGCGTGGATAGCGGTTTGACTCACGGGGATTTCCAAGTCTCC |
| | | ACCCCATTGACGTCAATGGGAGTTTGTTTTGGCACCAAAATCAAC |
| | | GGGACTTTCCAAAATGTCGTAACAACTCCGCCCCATTGACGCAAA |
| | | TGGGCGGTAGGCGTGTACGGTGGGAGGTCTATATAAGCAGAGCTC |
| | | TCTGGCTAACTAGAGAACCCACTGCTTACTGGCTTATCGAAATTA |
| | | ATACGACTCACTATAAGGGGGCGCTCGAGCAGGTTCAGAAGGAGA |
| | | TCAAAAACCCCCAAGGATCAAACATGCCAAAAAAGAAGAGAAAGG |
| | | TACCGAAGAAAAAAAGAAAGGTATACAATCACGATCAGGAGTTCG |
| | | ACCCCCCTAAGGTGTACCCACCAGTGCCTGCAGAGAAGAGGAAGC |
| | | CAATCCGGGTGCTGAGCCTGTTTGATGGCATCGCCACCGGCCTGC |
| | | TGGTGCTGAAGGATCTGGGCATCCAGGTGGACCGGTACATCGCCT |
| | | CCGAGGTGTGCGAGGATTCTATCACCGTGGGCATGGTGCGCCACC |
| | | AGGGCAAGATCATGTATGTGGGCGACGTGCGGTCCGTGACACAGA |
| | | AGCACATCCAGGAGTGGGGCCCATTCGATCTGGTGATCGGCGGCA |
| | | GCCCCTGTAATGACCTGTCCATCGTGAACCCTGCAAGGAAGGGAC |
| | | TGTACGAGGGAACCGGCCGGCTGTTCTTTGAGTTTTATAGACTGC |
| | | TGCACGACGCCAGGCCTAAGGAGGGCGACGATAGACCATTCTTTT |
| | | GGCTGTTCGAGAATGTGGTGGCTATGGGCGTGAGCGATAAGAGGG |
| | | ACATCTCCAGGTTTCTGGAGTCTAACCCCGTGATGATCGATGCAA |
| | | AGGAGGTGTCCGCCGCACACAGAGCCAGGTATTTCTGGGGCAATC |
| | | TGCCAGGAATGAACAGGCCACTGGCAAGCACCGTGAATGACAAGC |
| | | TGGAGCTGCAGGAGTGCCTGGAGCACGGAAGGATCGCCAAGTTTT |
| | | CCAAGGTGCGCACAATCACCACACGGAGCAATTCCATCAAGCAGG |
| | | GCAAGGATCAGCACTTCCCCGTGTTCATGAACGAGAAGGAGGACA |
| | | TCCTGTGGTGTACCGAGATGGAGAGAGTGTTCGGCTTTCCAGTGC |
| | | ACTACACAGACGTGTCTAACATGAGCAGGCTGGCAAGGCAGCGGC |
| | | TGCTGGGCAGATCTTGGAGCGTGCCCGTGATCAGGCACCTGTTCG |
| | | CCCCTCTGAAGGAGTATTTTGCCTGCGTGAGCAGCGGCAACTCCA |
| | | ATGCCAACAGCCGGGGCCCCTCTTTCAGCTCCGGATTGGTGCCTC |
| | | TGAGCCTGAGGGGCTCCCACATGGCAGCAATCCCCGCCCTGGACC |
| | | CCGAGGCCGAGCCTAGCATGGACGTGATCCTGGTGGGCTCTAGCG |
| | | AGCTGTCCTCTAGCGTGTCTCCAGGAACCGGAAGGGATCTGATCG |
| | | CATACGAGGTGAAGGCCAATCAGCGGAACATCGAGGACATCTGTA |
| | | TCTGCTGTGGCAGCCTGCAGGTGCACACACAGCACCCACTGTTCG |
| | | AGGGAGGAATCTGCGCACCCTGTAAGGATAAGTTCCTGGACGCCC |
| | | TGTTTCTGTACGACGATGACGGCTACCAGTCCTATTGCTCTATCT |
| | | GCTGTTCCGGCGAGACCCTGCTGATCTGCGGCAATCCAGATTGTA |
| | | CAAGGTGCTATTGTTTTGAGTGCGTGGACTCTCTGGTGGGACCAG |
| | | GCACCAGCGGAAAGGTGCACGCCATGTCCAACTGGGTGTGCTACC |
| | | TGTGCCTGCCATCCTCTCGCAGCGGACTGCTGCAGCGGAGAAGGA |
| | | AGTGGAGATCCCAGCTGAAGGCCTTCTATGATAGGGAGTCTGAGA |
| | | ACCCCCTGGAGATGTTTGAGACCGTGCCAGTGTGGCGCCGGCAGC |
| | | CCGTGAGGGTGCTGAGCCTGTTCGAGGATATCAAGAAGGAGCTGA |
| | | CATCCCTGGGCTTTCTGGAGTCCGGCTCTGACCCCGGACAGCTGA |
| | | AGCACGTGGTGGATGTGACCGACACAGTGCGGAAGGATGTGGAGG |
| | | AGTGGGGCCCTTTCGACCTGGTGTACGGAGCAACCCCTCCACTGG |
| | | GACACACATGCGACAGACCCCCTTCTTGGTACCTGTTCCAGTTTC |
| | | ACCGCCTGCTGCAGTATGCAAGGCCAAAGCCAGGCAGCCCTAGAC |
| | | CATTCTTTTGGATGTTCGTGGATAATCTGGTGCTGAACAAGGAGG |
| | | ATCTGGACGTGGCCAGCAGGTTTCTGGAGATGGAGCCAGTGACCA |
| | | TCCCAGACGTGCACGGCGGCTCCCTGCAGAATGCCGTGCGCGTGT |
| | | GGTCTAACATCCCTGCCATCAGAAGCAGGCACTGGGCACTGGTGA |
| | | GCGAGGAGGAGCTGTCCCTGCTGGCCCAGAATAAGCAGAGCAGCA |
| | | AGCTGGCCGCCAAGTGGCCTACAAAGCTGGTGAAGAACTGCTTCC |
| | | TGCCACTGCGGGAGTACTTCAAGTATTTTTCCACCGAGCTGACAT |
| | | CTAGCCTGGGAGGACCCTCCTCTGGCGCCCCACCACCTAGCGGCG |
| | | GCTCCCCTGCCGGCTCTCCAACCAGCACAGAGGAGGGCACCAGCG |
| | | AGTCCGCCACACCAGAGTCTGGACCTGGCACCAGCACAGAGCCAT |
| | | CCGAGGGCTCTGCCCCAGGCTCTCCTGCAGGCAGCCCTACCTCCA |
| | | CCGAAGAGGGCACCAGCACAGAGCCTTCTGAGGGCAGCGCCCCAG |
| | | GCACCTCTACAGAGCCAAGCGAGGAGAGACCTTTTCAGTGCCGGA |
| | | TTTGTATGCGCAATTTCTCCCACAAGCAGCACCGCGACGCCCATA |
| | | TTAGAACACATACTGGAGAAAAGCCCTTCGCTTGCGACATCTGTG |
| | | GACGGAAGTTCGCCCGCTCCGCCAACCTGACCCGCCACACTAAAA |
| | | TCCATACCGGCAGCCAAAAGCCATTCCAATGTCGCATCTGTATGC |
| | | GGAACTTTAGCCGCTCCGACAACCTGTCCGAGCATATTCGGACTC |
| | | ACACAGGGGAGAAACCATTTGCATGTGATATCTGCGGCAGAAAAT |
| | | TCGCTACCTCCGCCAACCTGTCCCGCCATACCAAGATACACACGG |
| | | GATCTCAGAAGCCCTTCCAGTGTCGAATCTGCATGCGTAACTTCA |
| | | GTATCCGCTCCACCCTGCGCGACCACATCCGCACCCACACCGGCG |
| | | AGAAGCCTTTTGCCTGTGACATTTGTGGGAGGAAATTTGCCCGCA |
| | | CCCCGGTGCGCATGGGCCATACCAAGATACACCTGCGCCAAAAAG |
| | | ATGCGGCCCGGGGATCTGGTGTTGATGGCTCCTCTGGTTCTGAAA |
| | | CTCCTGGTACTTCTGAGTCTGCTACTCCCGAATCTTCCCGGCCAG |
| | | GGGAACGGCCCTTCCAGTGTCGGATCTGCATGAGAAACTTTTCAA |
| | | CACCCTCAAAGCTGGATAGGCATACACGCACCCACACTGGAGAGA |
| | | AACCCTTTCAGTGCAGGATATGTATGCGGAATTTTTCCCTGGCTG |
| | | AGAACCTCCGAAGGCACCTGAGAACACATACCGGGAGTCAGAAGC |
| | | CTTTCCAATGCCGGATTTGCATGAGGAACTTCTCCCGCCAAGATA |
| | | ACCTGGGCCGCCACCTGCGCACACATACAGGCGAGAAGCCATTCC |
| | | AGTGTAGGATCTGCATGCGCAATTTTAGCGACGGCGGTAACTTGG |
| | | GACGCCACCTCAAAACGCATACAGGTGGCGGTGGCTCCCAGAAGC |
| | | CCTTTCAGTGCAGGATCTGCATGAGGAATTTTAGTCAGTCCCCAC |
| | | ATCTTAAAAGACACTTGCGGACGCATACTGGAGAGAAGCCCTTTC |
| | | AGTGTAGGATTTGTATGCGGAACTTCAGCCAGAGCACAAGCCTGC |
| | | AACGCCATCTCAAAACTCATTTGCGCGGGTCTAGCCCCAAGAAGA |
| | | AGAGAAAGGTGGGAGTCGACGGATCCAGCGGCTCCGAGACCCCAG |
| | | GCACATCTGAGAGCGCCACCCCTGAGTCCACCGGTGACTCCGTTG |
| | | CTTTCGAGGACGTGGCCGTGAACTTCACACTTGAGGAATGGGCCT |
| | | TGCTCGACCCAAGTCAGAAGAATCTGTACAGAGACGTGATGCGGG |
| | | AGACATTCAGGAATCTCGCCAGTGTCGGAAAGCAGTGGGAAGACC |
| | | AGAACATCGAAGATCCTTTCAAGATACCACGGCGCAATATCTCCC |
| | | ACATTCCTGAGAGGCTGTGTGAATCTAAGGAAGGCGGACAAGGTG |
| | | AGGAAAGCGCTGATTACAAAGATGATGACGATAAAGCCCCCAAGA |
| | | AGAAAAGGAAGGTCCCAAAGAAAAAAAGAAAGGTGTGAGGATCCT |
| | | GAGTCTAGAAATCAACCTCTGGATTACAAAATTTGTGAAAGATTG |
| | | ACTGGTATTCTTAACTATGTTGCTCCTTTTACGCTATGTGGATAC |
| | | GCTGCTTTAATGCCTTTGTATCATGCTATTGCTTCCCGTATGGCT |
| | | TTCATTTTCTCCTCCTTGTATAAATCCTGGTTGCTGTCTCTTTAT |
| | | GAGGAGTTGTGGCCCGTTGTCAGGCAACGTGGCGTGGTGTGCACT |
| | | GTGTTTGCTGACGCAACCCCCACTGGTTGGGGCATTGCCACCACC |
| | | TGTCAGCTCCTTTCCGGGACTTTCGCTTTCCCCCTCCCTATTGCC |
| | | ACGGCGGAACTCATCGCCGCCTGCCTTGCCCGCTGCTGGACAGGG |
| | | GCTCGGCTGTTGGGCACTGACAATTCCGTGGTGTTGTCGGGGAAA |
| | | TCATCGTCCTTTCCTTGGCTGCTCGCCTGTGTTGCCACCTGGATT |
| | | CTGCGCGGGACGTCCTTCTGCTACGTCCCTTCGGCCCTCAATCCA |
| | | GCGGACCTTCCTTCCCGCGGCCTGCTGCCGGCTCTGCGGCCTCTT |
| | | CCGCGTCTTCGCCTTCGCCCTCAGACGAGTCGGATCTCCCTTTGG |
| | | GCCGCCTCCCCGCCTGTTAATT |
| 26 | Full Nucleic Acid | GACATTGATTATTGACTAGTTATTAATAGTAATCAATTACGGGGT |
| | Sequence of | CATTAGTTCATAGCCCATATATGGAGTTCCGCGTTACATAACTTA |
| | Promoter and T7 | CGGTAAATGGCCCGCCTGGCTGACCGCCCAACGACCCCCGCCCAT |
| | | TGACGTCAATAATGACGTATGTTCCCATAGTAACGCCAATAGGGA |
| | | CTTTCCATTGACGTCAATGGGTGGAGTATTTACGGTAAACTGCCC |
| | | ACTTGGCAGTACATCAAGTGTATCATATGCCAAGTACGCCCCCTA |
| | | TTGACGTCAATGACGGTAAATGGCCCGCCTGGCATTATGCCCAGT |
| | | ACATGACCTTATGGGACTTTCCTACTTGGCAGTACATCTACGTAT |
| | | TAGTCATCGCTATTACCATGGTGATGCGGTTTTGGCAGTACATCA |
| | | ATGGGCGTGGATAGCGGTTTGACTCACGGGGATTTCCAAGTCTCC |
| | | ACCCCATTGACGTCAATGGGAGTTTGTTTTGGCACCAAAATCAAC |
| | | GGGACTTTCCAAAATGTCGTAACAACTCCGCCCCATTGACGCAAA |
| | | TGGGCGGTAGGCGTGTACGGTGGGAGGTCTATATAAGCAGAGCTC |
| | | TCTGGCTAACTAGAGAACCCACTGCTTACTGGCTTATCGAAATTA |
| | | ATACGACTCACTATAAGG |
| 27 | Full Nucleic Acid | GGGCGCTCGAGCAGGTTCAGAAGGAGATCAAAAACCCCCAAGGAT |
| | Sequence of 5′ UTR | CAAAC |
| 28 | Amino Acid | YNHDQEFDPPKVYPPVPAEKRKPIRVLSLFDGIATGLLVLKDLGI |
| | Sequence of DNA | QVDRYIASEVCEDSITVGMVRHQGKIMYVGDVRSVTQKHIQEWGP |
| | Methylation Domain | FDLVIGGSPCNDLSIVNPARKGLYEGTGRLFFEFYRLLHDARPKE |
| | (hDNMT3A) | GDDRPFFWLFENVVAMGVSDKRDISRFLESNPVMIDAKEVSAAHR |
| | | ARYFWGNLPGMNRPLASTVNDKLELQECLEHGRIAKFSKVRTITT |
| | | RSNSIKQGKDQHFPVFMNEKEDILWCTEMERVFGFPVHYTDVSNM |
| | | SRLARQRLLGRSWSVPVIRHLFAPLKEYFACV |
| 29 | Amino Acid | MAAIPALDPEAEPSMDVILVGSSELSSSVSPGTGRDLIAYEVKAN |
| | Sequence of DNA | QRNIEDICICCGSLQVHTQHPLFEGGICAPCKDKFLDALFLYDDD |
| | Methylation Domain | GYQSYCSICCSGETLLICGNPDCTRCYCFECVDSLVGPGTSGKVH |
| | (hDNMT3L) | AMSNWVCYLCLPSSRSGLLQRRRKWRSQLKAFYDRESENPLEMFE |
| | | TVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYA |
| | | RPKPGSPRPFFWMFVDNLVLNKEDLDVASRFLEMEPVTIPDVHGG |
| | | SLQNAVRVWSNIPAIRSRHWALVSEEELSLLAQNKQSSKLAAKWP |
| | | TKLVKNCFLPLREYFKYFSTELTSSL |
| 30 | Amino Acid | GVDGSSGSETPGTSESATPES |
| | Sequence of Linker | |
| | Comprising | |
| | XTEN16 | |
| 31 | Amino Acid | DYKDDDDK |
| | Sequence of FLAG | |
| | TAG | |
| 32 | Coding Sequence of | CCGAAGAAAAAAAGAAAGGTA |
| | Nuclear Localization | |
| | Signal | |
| 33 | ZIM3 | MNNSQGRVTFEDVTVNFTQGEWQRLNPEQRNLYRDVMLENYSNLV |
| | | SVGQGETTKPDVILRLEQGKEPWLEEEEVLGSGRAEKNGDIGGQI |
| | | WKPKDVKESL |
| 34 | ZNF436 | MAATLLMAGSQAPVTFEDMAMYLTREEWRPLDAAQRDLYRDVMQE |
| | | NYGNVVSLDFEIRSENEVNPKQEISEDVQFGTTSERPAENAEENP |
| | | ESEEGFESGDRSERQW |
| 35 | ZNF257 | MLENYRNLVFLGIAVSKPDLITCLEQGKEPCNMKRHEMVAKPPVM |
| | | CSHIAEDLCPERDIKYFFQKVILRRYDKCEHENLQLRKGCKSVDE |
| | | CKVCK |
| 36 | ZNF675 | MGLLTFRDVAIEFSLEEWQCLDTAQRNLYKNVILENYRNLVFLGI |
| | | AVSKQDLITCLEQEKEPLTVKRHEMVNEPPVMCSHFAQEFWPEQN |
| | | IKDSF |
| 37 | ZNF490 | MLQMQNSEHHGQSIKTQTDSISLEDVAVNFTLEEWALLDPGQRNI |
| | | YRDVMRATFKNLACIGEKWKDQDIEDEHKNQGRNLRSPMVEALCE |
| | | NKEDCPCGKSTSQIPDLNINLETPTG |
| 38 | ZNF320 | MALSQGLLTFRDVAIEFSQEEWKCLDPAQRTLYRDVMLENYRNLV |
| | | SLDISSKCMMNTLSSTGQGNTEVIHTGTLQRQASYHIGAFCSQEI |
| | | EKDIHDFVFQ |
| 39 | ZNF331 | MAQGLVTFADVAIDFSQEEWACLNSAQRDLYWDVMLENYSNLVSL |
| | | DLESAYENKSLPTKKNIHEIRASKRNSDRRSKSLGRNWICEGTLE |
| | | RPQRSRGR |
| 40 | ZNF816 | MLREEATKKSKEKEPGMALPQGRLTFRDVAIEFSLEEWKCLNPAQ |
| | | RALYRAVMLENYRNLEFVDSSLKSMMEFSSTRHSITGEVIHTGTL |
| | | QRHKSHHIGDFCFPEMKKDIHHFEFQWQ |
| 41 | ZNF680 | MPGPPGSLEMGPLTFRDVAIEFSLEEWQCLDTAQRNLYRKVMFEN |
| | | YRNLVFLGIAVSKPHLITCLEQGKEPWNRKRQEMVAKPPVIYSHF |
| | | TEDLWPEHSIKDSF |
| 42 | ZNF41 | MSPPWSPALAAEGRGSSCEASVSFEDVTVDFSKEEWQHLDPAQRR |
| | | LYWDVTLENYSHLLSVGYQIPKSEAAFKLEQGEGPWMLEGEAPHQ |
| | | SCSGEAIGKMQQQGIPGGIFFHC |
| 43 | ZNF189 | MASPSPPPESKEEWDYLDPAQRSLYKDVMMENYGNLVSLDVLNRD |
| | | KDEEPTVKQEIEEIEEEVEPQGVIVTRIKSEIDQDPMGRETFELV |
| | | GRLDKQRGIFLWEIPRESL |
| 44 | ZNF528 | MALTQGPLKFMDVAIEFSQEEWKCLDPAQRTLYRDVMLENYRNLV |
| | | SLGICLPDLSVTSMLEQKRDPWTLQSEEKIANDPDGRECIKGVNT |
| | | ERSSKLGSN |
| 45 | ZNF543 | MAASAQVSVTFEDVAVTFTQEEWGQLDAAQRTLYQEVMLETCGLL |
| | | MSLGCPLFKPELIYQLDHRQELWMATKDLSQSSYPGDNTKPKTTE |
| | | PTFSHLALPE |
| 46 | ZNF554 | MFSQEERMAAGYLPRWSQELVTFEDVSMDFSQEEWELLEPAQKNL |
| | | YREVMLENYRNVVSLEALKNQCTDVGIKEGPLSPAQTSQVTSLSS |
| | | WTGYLLFQPVASSHLEQREALWIEEKGTPQASCSDWMTVLRNQDS |
| | | TYKKVALQE |
| 47 | ZNF140 | MSQGSVTFRDVAIDFSQEEWKWLQPAQRDLYRCVMLENYGHLVSL |
| | | GLSISKPDVVSLLEQGKEPWLGKREVKRDLFSVSESSGEIKDFSP |
| | | KNVIYDD |
| 48 | ZNF610 | MEEAQKRKAKESGMALPQGRLTFMDVAIEFSQEEWKSLDPGQRAL |
| | | YRDVMLENYRNLVFLGRSCVLGSNAENKPIKNQLGLTLESHLSEL |
| | | QLFQAGRKIYRSNQVEKFTNHR |
| 49 | ZNF264 | MAAAVLTDRAQVSVTFDDVAVTFTKEEWGQLDLAQRTLYQEVMLE |
| | | NCGLLVSLGCPVPKAELICHLEHGQEPWIRKEDLSQDTCPGDKGK |
| | | PKTTEPTTCEPALSE |
| 50 | ZNF350 | MIQAQESITLEDVAVDFTWEEWQLLGAAQKDLYRDVMLENYSNLV |
| | | AVGYQASKPDALFKLEQGEQLWTIEDGIHSGACSDIWKVDHVLER |
| | | LQSESLVNR |
| 51 | ZNF8 | MEGVAGVMSVGPPAARLQEPVTFRDVAVDFTQEEWGQLDPTQRIL |
| | | YRDVMLETFGHLLSIGPELPKPEVISQLEQGTELWVAERGTTQGC |
| | | HPAWEPRSESQASRKEEGLPEE |
| 52 | ZNF582 | MSLGSELFRDVAIVFSQEEWQWLAPAQRDLYRDVMLETYSNLVSL |
| | | GLAVSKPDVISFLEQGKEPWMVERVVSGGLCPVLESRYDTKELFP |
| | | KQHVYEV |
| 53 | ZNF30 | MAHKYVGLQYHGSVTFEDVAIAFSQQEWESLDSSQRGLYRDVMLE |
| | | NYRNLVSMAGHSRSKPHVIALLEQWKEPEVTVRKDGRRWCTDLQL |
| | | EDDTIGCKEMPTSEN |
| 54 | ZNF324 | MAFEDVAVYFSQEEWGLLDTAQRALYRRVMLDNFALVASLGLSTS |
| | | RPRVVIQLERGEEPWVPSGTDTTLSRTTYRRRNPGSWSLTEDRDV |
| | | SG |
| 55 | ZNF98 | MLENYRNLVFVGIAASKPDLITCLEQGKEPWNVKRHEMVTEPPVV |
| | | YSYFAQDLWPKQGKKNYFQKVILRTYKKCGRENLQLRKYCKSMDE |
| | | CKVHKECYNGLNQC |
| 56 | ZNF669 | MHFRRPDPCREPLASPIQDSVAFEDVAVNFTQEEWALLDSSQKNL |
| | | YREVMQETCRNLASVGSQWKDQNIEDHFEKPGKDIRNHIVQRLCE |
| | | SKEDGQYGEVVSQIPNLDLNENISTGLKPCECSICGK |
| 57 | ZNF677 | MALSQGLFTFKDVAIEFSQEEWECLDPAQRALYRDVMLENYRNLL |
| | | SLDEDNIPPEDDISVGFTSKGLSPKENNKEELYHLVILERKESHG |
| | | INNFDLKEVWENMPKFDSLW |
| 58 | ZNF596 | MTFEDIIVDFTQEEWALLDTSQRKLFQDVMLENISHLVSIGKQLC |
| | | KSVVLSQLEQVEKLSTQRISLLQGREVGIKHQEIPFIHHIYQKGT |
| | | STISTMRS |
| 59 | ZNF214 | MAVIFEDVTIIFTWEEWKFLDSSQKRLYREVMWENYTNVMSVENW |
| | | NESYKSQEEKFRYLEYENFSYWQGWWNAGAQMYENQNYGETVQGT |
| | | DSKDLTQQDRSQC |
| 60 | ZNF37A | MITSQGSVSFRDVTVGFTQEEWQHLDPAQRTLYRDVMLENYSHLV |
| | | SVGYCIPKPEVILKLEKGEEPWILEEKFPSQSHLELINTSRNYSI |
| | | MKFNEFNKG |
| 61 | ZNF34 | MFEDVAVYLSREEWGRLGPAQRGLYRDVMLETYGNLVSLGVGPAG |
| | | PKPGVISQLERGDEPWVLDVQGTSGKEHLRVNSPALGTRTEYKEL |
| | | TSQETFGEEDPQGSEPVEACDHIS |
| 62 | ZNF250 | METYGNVVSLGLPGSKPDIISQLERGEDPWVLDRKGAKKSQGLWS |
| | | DYSDNLKYDHTTACTQQDSLSCPWECETKGESQNTDLSPKPLISE |
| | | QTVILGKTPLGRIDQENNETKQ |
| 63 | ZNF547 | MAEMNPAQGHVVFEDVAIYFSQEEWGHLDEAQRLLYRDVMLENLA |
| | | LLSSLGCCHGAEDEEAPLEPGVSVGVSQVMAPKPCLSTQNTQPCE |
| | | TCSSLLKDILRL |
| 64 | ZNF273 | MLDNYRNLVFLGIAVSKPDLITCLEQGKEPCNMKRHAMVAKPPVV |
| | | CSHFAQDLWPKQGLKDS |
| 65 | ZNF354A | MAAGQREARPQVSLIFEDVAVLFTRDEWRKLAPSQRNLYRDVMLE |
| | | NYRNLVSLGLPFTKPKVISLLQQGEDPWEVEKDGSGVSSLGSKSS |
| | | HKTTKSTQTQDSSFQ |
| 66 | ZFP82 | MALRSVMFSDVSIDFSPEEWEYLDLEQKDLYRDVMLENYSNLVSL |
| | | GCFISKPDVISSLEQGKEPWKVVRKGRRQYPDLETKYETKKLSLE |
| | | NDIYEIN |
| 67 | ZNF224 | MTTFKEAMTFKDVAVVFTEEELGLLDLAQRKLYRDVMLENFRNLL |
| | | SVGHQAFHRDTFHFLREEKIWMMKTAIQREGNSGDKIQTEMETVS |
| | | EAGTHQEW |
| 68 | ZNF33A | MFQVEQKSQESVSFKDVTVGFTQEEWQHLDPSQRALYRDVMLENY |
| | | SNLVSVGYCVHKPEVIFRLQQGEEPWKQEEEFPSQSFPEVWTADH |
| | | LKERSQENQSKHL |
| 69 | ZNF45 | MTKSKEAVTFKDVAVVFSEEELQLLDLAQRKLYRDVMLENFRNVV |
| | | SVGHQSTPDGLPQLEREEKLWMMKMATQRDNSSGAKNLKEMETLQ |
| | | EVGLRYLP |
| 70 | ZNF175 | MSQKPQVLGPEKQDGSCEASVSFEDVTVDFSREEWQQLDPAQRCL |
| | | YRDVMLELYSHLFAVGYHIPNPEVIFRMLKEKEPRVEEAEVSHQR |
| | | CQEREFGLEIPQKEISKKASFQ |
| 71 | ZNF595 | MELVTFRDVAIEFSPEEWKCLDPAQQNLYRDVMLENYRNLVSLGF |
| | | VISNPDLVTCLEQIKEPCNLKIHETAAKPPAICSPFSQDLSPVQG |
| | | IEDSF |
| 72 | ZNF184 | MSTLLQGGHNLLSSASFQESVTFKDVIVDFTQEEWKQLDPGQRDL |
| | | FRDVTLENYTHLVSIGLQVSKPDVISQLEQGTEPWIMEPSIPVGT |
| | | CADWETRLENSVSAPEPDISEE |
| 73 | ZNF419 | MDPAQVPVAADLLTDHEEGYVTFEDVAVYFSQEEWRLLDDAQRLL |
| | | YRNVMLENFTLLASLGLASSKTHEITQLESWEEPFMPAWEVVTSA |
| | | IPRGCWHGAEAEEAPEQIASVG |
| 74 | ZFP28-1 | MKKLEAVGTGIEPKAMSQGLVTFGDVAVDFSQEEWEWLNPIQRNL |
| | | YRKVMLENYRNLASLGLCVSKPDVISSLEQGKEPWTVKRKMTRAW |
| | | CPDLKAVWKIKELPLKKDFCEG |
| 75 | ZFP28-2 | MSLLGEHWDYDALFETQPGLVTIKNLAVDFRQQLHPAQKNFCKNG |
| | | IWENNSDLGSAGHCVAKPDLVSLLEQEKEPWMVKRELTGSLFSGQ |
| | | RSVHETQELFPKQDSYAE |
| 76 | ZNF18 | MLALAASQPARLEERLIRDRDLGASLLPAAPQEQWRQLDSTQKEQ |
| | | YWDLILETYGKMVSGAGISHPKSDLINSIEFGEELAGIYLHVNEK |
| | | IPRPTCIGDRQENDKENLNLENH |
| 77 | ZNF213 | MEGRPGETTDTCFVSGVHGPVALGDIPFYFSREEWGTLDPAQRDL |
| | | FWDIKRENSRNTTLGFGLKGQSEKSLLQEMVPVVPGQTGSDVTVS |
| | | WSPEEAEAWESENRPRAALGPVVGARRGRPPTRRRQFRDLA |
| 78 | ZNF394 | MVAVVRALQRALDGTSSQGMVTFEDTAVSLTWEEWERLDPARRDE |
| | | CRESAQKDSGSTVPPSLESRVENKELIPMQQILEEAEPQGQLQEA |
| | | FQGKRPLFSKCGSTHEDRVEKQSGDP |
| 79 | ZFP1 | MNKSQGSVSFTDVTVDFTQEEWEQLDPSQRILYMDVMLENYSNLL |
| | | SVEVWKADDQMERDHRNPDEQARQFLILKNQTPIEERGDLFGKAL |
| | | NLNTDFVSLRQVPYKYDLYEKTL |
| 80 | ZFP14 | MAHGSVTFRDVAIDFSQEEWEFLDPAQRDLYRDVMWENYSNFISL |
| | | GPSISKPDVITLLDEERKEPGMVVREGTRRYCPDLESRYRINTLS |
| | | PEKDIYEIYSFQWDIMER |
| 81 | ZNF416 | MAAAVLRDSTSVPVTAEAKLMGFTQGCVTFEDVAIYFSQEEWGLL |
| | | DEAQRLLYRDVMLENFALITALVCWHGMEDEETPEQSVSVEGVPQ |
| | | VRTPEASPSTQKIQSCDMCVPFLTDILHLTDLPGQELYLTGACAV |
| | | FHQDQK |
| 82 | ZNF557 | MLPPTAASQREGHTEGGELVNELLKSWLKGLVTFEDVAVEFTQEE |
| | | WALLDPAQRTLYRDVMLENCRNLASLGNQVDKPRLISQLEQEDKV |
| | | MTEERGILSGTCPDVENPFKAKGLTPKLHVFRKEQSRNMKMER |
| 83 | ZNF566 | MAQESVMFSDVSVDFSQEEWECLNDDQRDLYRDVMLENYSNLVSM |
| | | GHSISKPNVISYLEQGKEPWLADRELTRGQWPVLESRCETKKLFL |
| | | KKEIYEIESTQWEIMEK |
| 84 | ZNF729 | MPGAPGSLEMGPLTFRDVTIEFSLEEWQCLDTVQQNLYRDVMLEN |
| | | YRNLVFLGMAVFKPDLITCLKQGKEPWNMKRHEMVTKPPVMRSHF |
| | | TQDLWPDQSTKDSFQEVILRTYAR |
| 85 | ZIM2 | MAGSQFPDFKHLGTFLVFEELVTFEDVLVDFSPEELSSLSAAQRN |
| | | LYREVMLENYRNLVSLGHQFSKPDIISRLEEEESYAMETDSRHTV |
| | | ICQGE |
| 86 | ZNF254 | MPGPPRSLEMGLLTFRDVAIEFSLEEWQHLDIAQQNLYRNVMLEN |
| | | YRNLAFLGIAVSKPDLITCLEQGKEPWNMKRHE |
| 87 | ZNF764 | MAPPLAPLPPRDPNGAGPEWREPGAVSFADVAVYFCREEWGCLRP |
| | | AQRALYRDVMRETYGHLSALGIGGNKPALISWVEEEAELWGPAAQ |
| | | DPE |
| 88 | ZNF785 | MGPPLAPRPAHVPGEAGPRRTRESRPGAVSFADVAVYFSPEEWEC |
| | | LRPAQRALYRDVMRETFGHLGALGFSVPKPAFISWVEGEVEAWSP |
| | | EAQDPDGESS |
| 89 | ZNF10 (KOX1) | MDAKSLTAWSRTLVTFKDVFVDFTREEWKLLDTAQQIVYRNVMLE |
| | | NYKNLVSLGYQLTKPDVILRLEKGEEPWLVEREIHQETHPDSETA |
| | | FEIKSSVSSRSIFKDKQSCDIKMEGMARNDLWYLSLEEVWKCRDQ |
| | | LDKYQENPERHLRQVAFTQKKVLTQERVSESGKYGGNCLLPAQLV |
| | | LREYFHKRDSHTKSLKHDLVLNGHQDSCASNSNECGQTFCQNIHL |
| | | IQFARTHTGDKSYKCPDNDNSLTHGSSLGISKGIHREKPYECKEC |
| | | GKFFSWRSNLTRHQLIHTGEKPYECKECGKSFSRSSHLIGHQKTH |
| | | TGEEPYECKECGKSFSWFSHLVTHQRTHTGDKLYTCNQCGKSFVH |
| | | SSRLIRHQRTHTGEKPYECPECGKSFRQSTHLILHQRTHVRVRPY |
| | | ECNECGKSYSQRSHLVVHHRIHTGLKPFECKDCGKCFSRSSHLYS |
| | | HQRTHTGEKPYECHDCGKSFSQSSALIVHQRIHTGEKPYECCQCG |
| | | KAFIRKNDLIKHQRIHVGEETYKCNQCGIIFSQNSPFIVHQIAHT |
| | | GEQFLTCNQCGTALVNTSNLIGYQTNHIRENAY |
| 90 | CBX5 | MGKKTKRTADSSSSEDEEEYVVEKVLDRRVVKGQVEYLLKWKGFS |
| | (chromoshadow | EEHNTWEPEKNLDCPELISEFMKKYKKMKEGENNKPREKSESNKR |
| | domain) | KSNFSNSADDIKSKKKREQSNDIARGFERGLEPEKIIGATDSCGD |
| | | LMFLMKWKDTDEADLVLAKEANVKCPQIVIAFYEERLTWHAYPED |
| | | AENKEKETAKS |
| 91 | RYBP | MTMGDKKSPTRPKRQAKPAADEGFWDCSVCTFRNSAEAFKCSICD |
| | (YAF2_RYBP | VRKGTSTRKPRINSQLVAQQVAQQYATPPPPKKEKKEKVEKQDKE |
| | component of | KPEKDKEISPSVTKKNTNKKTKPKSDILKDPPSEANSIQSANATT |
| | PRC1) | KTSETNHTSRPRLKNVDRSTAQQLAVTVGNVTVIITDFKEKTRSS |
| | | STSSSTVTSSAGSEQQNQSSSGSESTDKGSSRSSTPKGDMSAVND |
| | | ESF |
| 92 | YAF2 | MGDKKSPTRPKRQPKPSSDEGYWDCSVCIFRNSAEAFKCMMCDVR |
| | (YAF2_RYBP | KGTSTRKPRPVSQLVAQQVTQQFVPPTQSKKEKKDKVEKEKSEKE |
| | component of | TTSKKNSHKKTRPRLKNVDRSSAQHLEVTVGDLIVIITDFKEKTK |
| | PRC1) | SPPASSAASADQHSQSGSSSDNTERGMSRSSSPRGEASSLNGESH |
| 93 | MGA (component | MEEKQQIILANQDGGTVAGAAPTFFVILKQPGNGKTDQGILVINQ |
| | of PRC1.6) | DACALASSVSSPVKSKGKICLPADCTVGGITVILDNNSMWNEFYH |
| | | RSTEMILTKQGRRMFPYCRYWITGLDSNLKYILVMDISPVDNHRY |
| | | KWNGRWWEPSGKAEPHVLGRVFIHPESPSTGHYWMHQPVSFYKLK |
| | | LINNTLDQEGHIILHSMHRYLPRLHLVPAEKAVEVIQLNGPGVHT |
| | | FTFPQTEFFAVTAYQNIQITQLKIDYNPFAKGFRDDGLNNKPQRD |
| | | GKQKNSSDQEGNNISSSSGHRVRLTEGQGSEIQPGDLDPLSRGHE |
| | | TSGKGLEKTSLNIKRDFLGFMDTDSALSEVPQLKQEISECLIASS |
| | | FEDDSRVASPLDQNGSFNVVIKEEPLDDYDYELGECPEGVTVKQE |
| | | ETDEETDVYSNSDDDPILEKQLKRHNKVDNPEADHLSSKWLPSSP |
| | | SGVAKAKMFKLDTGKMPVVYLEPCAVTRSTVKISELPDNMLSTSR |
| | | KDKSSMLAELEYLPTYIENSNETAFCLGKESENGLRKHSPDLRVV |
| | | QKYPLLKEPQWKYPDISDSISTERILDDSKDSVGDSLSGKEDLGR |
| | | KRTTMLKIATAAKVVNANQNASPNVPGKRGRPRKLKLCKAGRPPK |
| | | NTGKSLISTKNTPVSPGSTFPDVKPDLEDVDGVLFVSFESKEALD |
| | | IHAVDGTTEESSSLQASTINDSGYRARISQLEKELIEDLKTLRHK |
| | | QVIHPGLQEVGLKLNSVDPTMSIDLKYLGVQLPLAPATSFPFWNL |
| | | TGTNPASPDAGFPFVSRTGKINDFTKIKGWRGKFHSASASRNEGG |
| | | NSESSLKNRSAFCSDKLDEYLENEGKLMETSMGFSSNAPTSPVVY |
| | | QLPTKSTSYVRTLDSVLKKQSTISPSTSYSLKPHSVPPVSRKAKS |
| | | QNRQATFSGRIKSSYKSILPYPVSPKQKYSHVILGDKVTKNSSGI |
| | | ISENQANNFVVPILDENIFPKQISLRQAQQQQQQQQGSRPPGLSK |
| | | SQVKLMDLEDCALWEGKPRTYITEERADVSLTTLLTAQASLKTKP |
| | | IHTIIRKRAPPCNNDFCRLGCVCSSLALEKRQPAHCRRPDCMFGC |
| | | TCLKRKVVLVKGGSKIKHFQRKAAHRDPVFYDTLGEEAREEEEGI |
| | | REEEEQLKEKKKRKKLEYTICETEPEQPVRHYPLWVKVEGEVDPE |
| | | PVYIPTPSVIEPMKPLLLPQPEVLSPTVKGKLLIGIKSPRSYTPK |
| | | PNPVIREEDKDPVYLYFESMMTCARVRVYERKKEDQRQPSSSSSP |
| | | SPSFQQQTSCHSSPENHNNAKEPDSEQQPLKQLTCDLEDDSDKLQ |
| | | EKSWKSSCNEGESSSTSYMHQRSPGGPTKLIEIISDCNWEEDRNK |
| | | ILSILSQHINSNMPQSLKVGSFIIELASQRKSRGEKNPPVYSSRV |
| | | KISMPSCQDQDDMAEKSGSETPDGPLSPGKMEDISPVQTDALDSV |
| | | RERLHGGKGLPFYAGLSPAGKLVAYKRKPSSSTSGLIQVASNAKV |
| | | AASRKPRTLLPSTSNSKMASSSGTATNRPGKNLKAFVPAKRPIAA |
| | | RPSPGGVFTQFVMSKVGALQQKIPGVSTPQTLAGTQKFSIRPSPV |
| | | MVVTPVVSSEPVQVCSPVTAAVTTTTPQVFLENTTAVTPMTAISD |
| | | VETKETTYSSGATTTGVVEVSETNTSTSVTSTQSTATVNLIKTTG |
| | | ITTPVASVAFPKSLVASPSTITLPVASTASTSLVVVTAAASSSMV |
| | | TTPTSSLGSVPIILSGINGSPPVSQRPENAAQIPVATPQVSPNTV |
| | | KRAGPRLLLIPVQQGSPTLRPVSNTQLQGHRMVLQPVRSPSGMNL |
| | | FRHPNGQIVQLLPLHQLRGSNTQPNLQPVMFRNPGSVMGIRLPAP |
| | | SKPSETPPSSTSSSAFSVMNPVIQAVGSSSAVNVITQAPSLLSSG |
| | | ASFVSQAGTLTLRISPPEPQSFASKTGSETKITYSSGGQPVGTAS |
| | | LIPLQSGSFALLQLPGQKPVPSSILQHVASLQMKRESQNPDQKDE |
| | | TNSIKREQETKKVLQSEGEAVDPEANVIKQNSGAATSEETLNDSL |
| | | EDRGDHLDEECLPEEGCATVKPSEHSCITGSHTDQDYKDVNEEYG |
| | | ARNRKSSKEKVAVLEVRTISEKASNKTVQNLSKVQHQKLGDVKVE |
| | | QQKGFDNPEENSSEFPVTFKEESKFELSGSKVMEQQSNLQPEAKE |
| | | KECGDSLEKDRERWRKHLKGPLTRKCVGASQECKKEADEQLIKET |
| | | KTCQENSDVFQQEQGISDLLGKSGITEDARVLKTECDSWSRISNP |
| | | SAFSIVPRRAAKSSRGNGHFQGHLLLPGEQIQPKQEKKGGRSSAD |
| | | FTVLDLEEDDEDDNEKTDDSIDEIVDVVSDYQSEEVDDVEKNNCV |
| | | EYIEDDEEHVDIETVEELSEEINVAHLKTTAAHTQSFKQPSCTHI |
| | | SADEKAAERSRKAPPIPLKLKPDYWSDKLQKEAEAFAYYRRTHTA |
| | | NERRRRGEMRDLFEKLKITLGLLHSSKVSKSLILTRAFSEIQGLT |
| | | DQADKLIGQKNLLTRKRNILIRKVSSLSGKTEEVVLKKLEYIYAK |
| | | QQALEAQKRKKKMGSDEFDISPRISKQQEGSSASSVDLGQMFINN |
| | | RRGKPLILSRKKDQATENTSPLNTPHTSANLVMTPQGQLLTLKGP |
| | | LFSGPVVAVSPDLLESDLKPQVAGSAVALPENDDLFMMPRIVNVT |
| | | SLATEGGLVDMGGSKYPHEVPDSKPSDHLKDTVRNEDNSLEDKGR |
| | | ISSRGNRDGRVTLGPTQVFLANKDSGYPQIVDVSNMQKAQEFLPK |
| | | KISGDMRGIQYKWKESESRGERVKSKDSSFHKLKMKDLKDSSIEM |
| | | ELRKVTSAIEEAALDSSELLINMEDEDDTDETLTSLLNEIAFLNQ |
| | | QLNDDSVGLAELPSSMDTEFPGDARRAFISKVPPGSRATFQVEHL |
| | | GTGLKELPDVQGESDSISPLLLHLEDDDFSENEKQLAEPASEPDV |
| | | LKIVIDSEIKDSLLSNKKAIDGGKNTSGLPAEPESVSSPPTLHMK |
| | | TGLENSNSTDTLWRPMPKLAPLGLKVANPSSDADGQSLKVMPCLA |
| | | PIAAKVGSVGHKMNLTGNDQEGRESKVMPTLAPVVAKLGNSGASP |
| | | SSAGK |
| 94 | CBX1 | MGKKQNKKKVEEVLEEEEEEYVVEKVLDRRVVKGKVEYLLKWKGF |
| | (chromoshadow) | SDEDNTWEPEENLDCPDLIAEFLQSQKTAHETDKSEGGKRKADSD |
| | | SEDKGEESKPKKKKEESEKPRGFARGLEPERIIGATDSSGELMFL |
| | | MKWKNSDEADLVPAKEANVKCPQVVISFYEERLTWHSYPSEDDDK |
| | | KDDKN |
| 95 | SCMH1 | MLVCYSVLACEILWDLPCSIMGSPLGHFTWDKYLKETCSVPAPVH |
| | (SAM_1/SPM) | CFKQSYTPPSNEFKISMKLEAQDPRNTTSTCIATVVGLTGARLRL |
| | | RLDGSDNKNDFWRLVDSAEIQPIGNCEKNGGMLQPPLGFRLNASS |
| | | WPMFLLKTLNGAEMAPIRIFHKEPPSPSHNFFKMGMKLEAVDRKN |
| | | PHFICPATIGEVRGSEVLVTFDGWRGAFDYWCRFDSRDIFPVGWC |
| | | SLTGDNLQPPGTKVVIPKNPYPASDVNTEKPSIHSSTKTVLEHQP |
| | | GQRGRKPGKKRGRIPKILISHPISAPSKTAEPLKFPKKRGPKPGS |
| | | KRKPRTLLNPPPASPTTSTPEPDTSTVPQDAATIPSSAMQAPTVC |
| | | IYLNKNGSTGPHLDKKKVQQLPDHFGPARASVVLQQAVQACIDCA |
| | | YHQKTVFSFLKQGHGGEVISAVFDREQHTLNLPAVNSITYVLRFL |
| | | EKLCHNLRSDNLFGNQPFTQTHLSLTAIEYSHSHDRYLPGETFVL |
| | | GNSLARSLEPHSDSMDSASNPTNLVSTSQRHRPLLSSCGLPPSTA |
| | | SAVRRLCSRGVLKGSNERRDMESFWKLNRSPGSDRYLESRDASRL |
| | | SGRDPSSWTVEDVMQFVREADPQLGPHADLERKHEIDGKALLLLR |
| | | SDMMMKYMGLKLGPALKLSYHIDRLKQGKF |
| 96 | MPP8 | MEQVAEGARVTAVPVSAADSTEELAEVEEGVGVVGEDNDAAARGA |
| | (Chromodomain) | EAFGDSEEDGEDVFEVEKILDMKTEGGKVLYKVRWKGYTSDDDTW |
| | | EPEIHLEDCKEVLLEFRKKIAENKAKAVRKDIQRLSLNNDIFEAN |
| | | SDSDQQSETKEDTSPKKKKKKLRQREEKSPDDLKKKKAKAGKLKD |
| | | KSKPDLESSLESLVFDLRTKKRISEAKEELKESKKPKKDEVKETK |
| | | ELKKVKKGEIRDLKTKTREDPKENRKTKKEKFVESQVESESSVLN |
| | | DSPFPEDDSEGLHSDSREEKQNTKSARERAGQDMGLEHGFEKPLD |
| | | SAMSAEEDTDVRGRRKKKTPRKAEDTRENRKLENKNAFLEKKTVP |
| | | KKQRNQDRSKSAAELEKLMPVSAQTPKGRRLSGEERGLWSTDSAE |
| | | EDKETKRNESKEKYQKRHDSDKEEKGRKEPKGLKTLKEIRNAFDL |
| | | FKLTPEEKNDVSENNRKREEIPLDFKTIDDHKTKENKQSLKERRN |
| | | TRDETDTWAYIAAEGDQEVLDSVCQADENSDGRQQILSLGMDLQL |
| | | EWMKLEDFQKHLDGKDENFAATDAIPSNVLRDAVKNGDYITVKVA |
| | | LNSNEEYNLDQEDSSGMTLVMLAAAGGQDDLLRLLITKGAKVNGR |
| | | QKNGTTALIHAAEKNFLTTVAILLEAGAFVNVQQSNGETALMKAC |
| | | KRGNSDIVRLVIECGADCNILSKHQNSALHFAKQSNNVLVYDLLK |
| | | NHLETLSRVAEETIKDYFEARLALLEPVFPIACHRLCEGPDFSTD |
| | | FNYKPPQNIPEGSGILLFIFHANFLGKEVIARLCGPCSVQAVVLN |
| | | DKFQLPVFLDSHFVYSFSPVAGPNKLFIRLTEAPSAKVKLLIGAY |
| | | RVQLQ |
| 97 | SUMO3 (Rad60- | MSEEKPKEGVKTENDHINLKVAGQDGSVVQFKIKRHTPLSKLMKA |
| | SLD) | YCERQGLSMRQIRFRFDGQPINETDTPAQLEMEDEDTIDVEQQQT |
| | | GGVPESSLAGHSF |
| 98 | HERC2 (Cyt-b5) | MPSESFCLAAQARLDSKWLKTDIQLAFTRDGLCGLWNEMVKDGEI |
| | | VYTGTESTQNGELPPRKDDSVEPSGTKKEDLNDKEKKDEEETPAP |
| | | IYRAKSILDSWVWGKQPDVNELKECLSVLVKEQQALAVQSATTTL |
| | | SALRLKQRLVILERYFIALNRTVFQENVKVKWKSSGISLPPVDKK |
| | | SSRPAGKGVEGLARVGSRAALSFAFAFLRRAWRSGEDADLCSELL |
| | | QESLDALRALPEASLFDESTVSSVWLEVVERATRFLRSVVTGDVH |
| | | GTPATKGPGSIPLQDQHLALAILLELAVQRGTLSQMLSAILLLLQ |
| | | LWDSGAQETDNERSAQGTSAPLLPLLQRFQSIICRKDAPHSEGDM |
| | | HLLSGPLSPNESFLRYLTLPQDNELAIDLRQTAVVVMAHLDRLAT |
| | | PCMPPLCSSPTSHKGSLQEVIGWGLIGWKYYANVIGPIQCEGLAN |
| | | LGVTQIACAEKRFLILSRNGRVYTQAYNSDTLAPQLVQGLASRNI |
| | | VKIAAHSDGHHYLALAATGEVYSWGCGDGGRLGHGDTVPLEEPKV |
| | | ISAFSGKQAGKHVVHIACGSTYSAAITAEGELYTWGRGNYGRLGH |
| | | GSSEDEAIPMLVAGLKGLKVIDVACGSGDAQTLAVTENGQVWSWG |
| | | DGDYGKLGRGGSDGCKTPKLIEKLQDLDVVKVRCGSQFSIALTKD |
| | | GQVYSWGKGDNQRLGHGTEEHVRYPKLLEGLQGKKVIDVAAGSTH |
| | | CLALTEDSEVHSWGSNDQCQHFDTLRVTKPEPAALPGLDTKHIVG |
| | | IACGPAQSFAWSSCSEWSIGLRVPFVVDICSMTFEQLDLLLRQVS |
| | | EGMDGSADWPPPQEKECVAVATLNLLRLQLHAAISHQVDPEFLGL |
| | | GLGSILLNSLKQTVVTLASSAGVLSTVQSAAQAVLQSGWSVLLPT |
| | | AEERARALSALLPCAVSGNEVNISPGRRFMIDLLVGSLMADGGLE |
| | | SALHAAITAEIQDIEAKKEAQKEKEIDEQEANASTFHRSRTPLDK |
| | | DLINTGICESSGKQCLPLVQLIQQLLRNIASQTVARLKDVARRIS |
| | | SCLDFEQHSRERSASLDLLLRFQRLLISKLYPGESIGQTSDISSP |
| | | ELMGVGSLLKKYTALLCTHIGDILPVAASIASTSWRHFAEVAYIV |
| | | EGDFTGVLLPELVVSIVLLLSKNAGLMQEAGAVPLLGGLLEHLDR |
| | | FNHLAPGKERDDHEELAWPGIMESFFTGQNCRNNEEVTLIRKADL |
| | | ENHNKDGGFWTVIDGKVYDIKDFQTQSLTGNSILAQFAGEDPVVA |
| | | LEAALQFEDTRESMHAFCVGQYLEPDQEIVTIPDLGSLSSPLIDT |
| | | ERNLGLLLGLHASYLAMSTPLSPVEIECAKWLQSSIFSGGLQTSQ |
| | | IHYSYNEEKDEDHCSSPGGTPASKSRLCSHRRALGDHSQAFLQAI |
| | | ADNNIQDHNVKDFLCQIERYCRQCHLTTPIMFPPEHPVEEVGRLL |
| | | LCCLLKHEDLGHVALSLVHAGALGIEQVKHRTLPKSVVDVCRVVY |
| | | QAKCSLIKTHQEQGRSYKEVCAPVIERLRFLFNELRPAVCNDLSI |
| | | MSKFKLLSSLPRWRRIAQKIIRERRKKRVPKKPESTDDEEKIGNE |
| | | ESDLEEACILPHSPINVDKRPIAIKSPKDKWQPLLSTVTGVHKYK |
| | | WLKQNVQGLYPQSPLLSTIAEFALKEEPVDVEKMRKCLLKQLERA |
| | | EVRLEGIDTILKLASKNFLLPSVQYAMFCGWQRLIPEGIDIGEPL |
| | | TDCLKDVDLIPPFNRMLLEVTFGKLYAWAVQNIRNVLMDASAKFK |
| | | ELGIQPVPLQTITNENPSGPSLGTIPQARFLLVMLSMLTLQHGAN |
| | | NLDLLLNSGMLALTQTALRLIGPSCDNVEEDMNASAQGASATVLE |
| | | ETRKETAPVQLPVSGPELAAMMKIGTRVMRGVDWKWGDQDGPPPG |
| | | LGRVIGELGEDGWIRVQWDIGSTNSYRMGKEGKYDLKLAELPAAA |
| | | QPSAEDSDTEDDSEAEQTERNIHPTAMMFTSTINLLQTLCLSAGV |
| | | HAEIMQSEATKTLCGLLRMLVESGTTDKTSSPNRLVYREQHRSWC |
| | | TLGFVRSIALTPQVCGALSSPQWITLLMKVVEGHAPFTATSLQRQ |
| | | ILAVHLLQAVLPSWDKTERARDMKCLVEKLFDFLGSLLTTCSSDV |
| | | PLLRESTLRRRRVRPQASLTATHSSTLAEEVVALLRTLHSLTQWN |
| | | GLINKYINSQLRSITHSFVGRPSEGAQLEDYFPDSENPEVGGLMA |
| | | VLAVIGGIDGRLRLGGQVMHDEFGEGTVTRITPKGKITVQFSDMR |
| | | TCRVCPLNQLKPLPAVAFNVNNLPFTEPMLSVWAQLVNLAGSKLE |
| | | KHKIKKSTKQAFAGQVDLDLLRCQQLKLYILKAGRALLSHQDKLR |
| | | QILSQPAVQETGTVHTDDGAVVSPDLGDMSPEGPQPPMILLQQLL |
| | | ASATQPSPVKAIFDKQELEAAALAVCQCLAVESTHPSSPGFEDCS |
| | | SSEATTPVAVQHIRPARVKRRKQSPVPALPIVVQLMEMGFSRRNI |
| | | EFALKSLTGASGNASSLPGVEALVGWLLDHSDIQVTELSDADTVS |
| | | DEYSDEEVVEDVDDAAYSMSTGAVVTESQTYKKRADFLSNDDYAV |
| | | YVRENIQVGMMVRCCRAYEEVCEGDVGKVIKLDRDGLHDLNVQCD |
| | | WQQKGGTYWVRYIHVELIGYPPPSSSSHIKIGDKVRVKASVTTPK |
| | | YKWGSVTHQSVGVVKAFSANGKDIIVDFPQQSHWTGLLSEMELVP |
| | | SIHPGVTCDGCQMFPINGSRFKCRNCDDFDFCETCFKTKKHNTRH |
| | | TFGRINEPGQSAVFCGRSGKQLKRCHSSQPGMLLDSWSRMVKSLN |
| | | VSSSVNQASRLIDGSEPCWQSSGSQGKHWIRLEIFPDVLVHRLKM |
| | | IVDPADSSYMPSLVVVSGGNSLNNLIELKIININPSDTTVPLLND |
| | | CTEYHRYIEIAIKQCRSSGIDCKIHGLILLGRIRAEEEDLAAVPF |
| | | LASDNEEEEDEKGNSGSLIRKKAAGLESAATIRTKVFVWGLNDKD |
| | | QLGGLKGSKIKVPSFSETLSALNVVQVAGGSKSLFAVTVEGKVYA |
| | | CGEATNGRLGLGISSGTVPIPRQITALSSYVVKKVAVHSGGRHAT |
| | | ALTVDGKVFSWGEGDDGKLGHFSRMNCDKPRLIEALKTKRIRDIA |
| | | CGSSHSAALTSSGELYTWGLGEYGRLGHGDNTTQLKPKMVKVLLG |
| | | HRVIQVACGSRDAQTLALTDEGLVFSWGDGDFGKLGRGGSEGCNI |
| | | PQNIERLNGQGVCQIECGAQFSLALTKSGVVWTWGKGDYFRLGHG |
| | | SDVHVRKPQVVEGLRGKKIVHVAVGALHCLAVTDSGQVYAWGDND |
| | | HGQQGNGTTTVNRKPTLVQGLEGQKITRVACGSSHSVAWTTVDVA |
| | | TPSVHEPVLFQTARDPLGASYLGVPSDADSSAASNKISGASNSKP |
| | | NRPSLAKILLSLDGNLAKQQALSHILTALQIMYARDAVVGALMPA |
| | | AMIAPVECPSFSSAAPSDASAMASPMNGEECMLAVDIEDRLSPNP |
| | | WQEKREIVSSEDAVTPSAVTPSAPSASARPFIPVTDDLGAASIIA |
| | | ETMTKTKEDVESQNKAAGPEPQALDEFTSLLIADDTRVVVDLLKL |
| | | SVCSRAGDRGRDVLSAVLSGMGTAYPQVADMLLELCVTELEDVAT |
| | | DSQSGRLSSQPVVVESSHPYTDDTSTSGTVKIPGAEGLRVEFDRQ |
| | | CSTERRHDPLTVMDGVNRIVSVRSGREWSDWSSELRIPGDELKWK |
| | | FISDGSVNGWGWRFTVYPIMPAAGPKELLSDRCVLSCPSMDLVTC |
| | | LLDFRLNLASNRSIVPRLAASLAACAQLSALAASHRMWALQRLRK |
| | | LLTTEFGQSININRLLGENDGETRALSFTGSALAALVKGLPEALQ |
| | | RQFEYEDPIVRGGKQLLHSPFFKVLVALACDLELDTLPCCAETHK |
| | | WAWERRYCMASRVAVALDKRTPLPRLFLDEVAKKIRELMADSENM |
| | | DVLHESHDIFKREQDEQLVQWMNRRPDDWILSAGGSGTIYGWGHN |
| | | HRGQLGGIEGAKVKVPTPCEALATLRPVQLIGGEQTLFAVTADGK |
| | | LYATGYGAGGRLGIGGTESVSTPTLLESIQHVFIKKVAVNSGGKH |
| | | CLALSSEGEVYSWGEAEDGKLGHGNRSPCDRPRVIESLRGIEVVD |
| | | VAAGGAHSACVTAAGDLYTWGKGRYGRLGHSDSEDQLKPKLVEAL |
| | | QGHRVVDIACGSGDAQTLCLTDDDTVWSWGDGDYGKLGRGGSDGC |
| | | KVPMKIDSLTGLGVVKVECGSQFSVALTKSGAVYTWGKGDYHRLG |
| | | HGSDDHVRRPRQVQGLQGKKVIAIATGSLHCVCCTEDGEVYTWGD |
| | | NDEGQLGDGITNAIQRPRLVAALQGKKVNRVACGSAHTLAWSTSK |
| | | PASAGKLPAQVPMEYNHLQEIPIIALRNRLLLLHHLSELFCPCIP |
| | | MFDLEGSLDETGLGPSVGFDTLRGILISQGKEAAFRKVVQATMVR |
| | | DRQHGPVVELNRIQVKRSRSKGGLAGPDGTKSVFGQMCAKMSSFG |
| | | PDSLLLPHRVWKVKFVGESVDDCGGGYSESIAEICEELQNGLTPL |
| | | LIVTPNGRDESGANRDCYLLSPAARAPVHSSMFRFLGVLLGIAIR |
| | | TGSPLSLNLAEPVWKQLAGMSLTIADLSEVDKDFIPGLMYIRDNE |
| | | ATSEEFEAMSLPFTVPSASGQDIQLSSKHTHITLDNRAEYVRLAI |
| | | NYRLHEFDEQVAAVREGMARVVPVPLLSLFTGYELETMVCGSPDI |
| | | PLHLLKSVATYKGIEPSASLIQWFWEVMESESNTERSLFLRFVWG |
| | | RTRLPRTIADFRGRDFVIQVLDKYNPPDHFLPESYTCFFLLKLPR |
| | | YSCKQVLEEKLKYAIHFCKSIDIDDYARIALTGEPAADDSSDDSD |
| | | NEDVDSFASDSTQDYLTGH |
| 99 | BIN1 (SH3_9) | MAEMGSKGVTAGKIASNVQKKLTRAQEKVLQKLGKADETKDEQFE |
| | | QCVQNFNKQLTEGTRLQKDLRTYLASVKAMHEASKKLNECLQEVY |
| | | EPDWPGRDEANKIAENNDLLWMDYHQKLVDQALLTMDTYLGQFPD |
| | | IKSRIAKRGRKLVDYDSARHHYESLQTAKKKDEAKIAKPVSLLEK |
| | | AAPQWCQGKLQAHLVAQTNLLRNQAEEELIKAQKVFEEMNVDLQE |
| | | ELPSLWNSRVGFYVNTFQSIAGLEENFHKEMSKLNQNLNDVLVGL |
| | | EKQHGSNTFTVKAQPSDNAPAKGNKSPSPPDGSPAATPEIRVNHE |
| | | PEPAGGATPGATLPKSPSQLRKGPPVPPPPKHTPSKEVKQEQILS |
| | | LFEDTFVPEISVTTPSQFEAPGPFSEQASLLDLDFDPLPPVTSPV |
| | | KAPTPSGQSIPWDLWEPTESPAGSLPSGEPSAAEGTFAVSWPSQT |
| | | AEPGPAQPAEASEVAGGTQPAAGAQEPGETAASEAASSSLPAVVV |
| | | ETFPATVNGTVEGGSGAGRLDLPPGFMFKVQAQHDYTATDTDELQ |
| | | LKAGDVVLVIPFQNPEEQDEGWLMGVKESDWNQHKELEKCRGVFP |
| | | ENFTERVP |
| 100 | PCGF2 (RING | MHRTTRIKITELNPHLMCALCGGYFIDATTIVECLHSFCKTCIVR |
| | finger protein | YLETNKYCPMCDVQVHKTRPLLSIRSDKTLQDIVYKLVPGLFKDE |
| | domain) | MKRRRDFYAAYPLTEVPNGSNEDRGEVLEQEKGALSDDEIVSLSI |
| | | EFYEGARDRDEKKGPLENGDGDKEKTGVRFLRCPAAMTVMHLAKF |
| | | LRNKMDVPSKYKVEVLYEDEPLKEYYTLMDIAYIYPWRRNGPLPL |
| | | KYRVQPACKRLILATVPTPSEGTNTSGASECESVSDKAPSPATLP |
| | | ATSSSLPSPATPSHGSPSSHGPPATHPTSPTPPSTASGATTAANG |
| | | GSLNCLQTPSSTSRGRKMTVNGAPVPPLT |
| 101 | TOX (HMG box) | MDVRFYPPPAQPAAAPDAPCLGPSPCLDPYYCNKFDGENMYMSMT |
| | | EPSQDYVPASQSYPGPSLESEDFNIPPITPPSLPDHSLVHLNEVE |
| | | SGYHSLCHPMNHNGLLPFHPQNMDLPEITVSNMLGQDGTLLSNSI |
| | | SVMPDIRNPEGTQYSSHPQMAAMRPRGQPADIRQQPGMMPHGQLT |
| | | TINQSQLSAQLGLNMGGSNVPHNSPSPPGSKSATPSPSSSVHEDE |
| | | GDDTSKINGGEKRPASDMGKKPKTPKKKKKKDPNEPQKPVSAYAL |
| | | FFRDTQAAIKGQNPNATFGEVSKIVASMWDGLGEEQKQVYKKKTE |
| | | AAKKEYLKQLAAYRASLVSKSYSEPVDVKTSQPPQLINSKPSVFH |
| | | GPSQAHSALYLSSHYHQQPGMNPHLTAMHPSLPRNIAPKPNNQMP |
| | | VTVSIANMAVSPPPPLQISPPLHQHLNMQQHQPLTMQQPLGNQLP |
| | | MQVQSALHSPTMQQGFTLQPDYQTIINPTSTAAQVVTQAMEYVRS |
| | | GCRNPPPQPVDWNNDYCSSGGMQRDKALYLT |
| 102 | FOXA1 (HNF3A C- | MLGTVKMEGHETSDWNSYYADTQEAYSSVPVSNMNSGLGSMNSMN |
| | terminal domain) | TYMTMNTMTTSGNMTPASFNMSYANPGLGAGLSPGAVAGMPGGSA |
| | | GAMNSMTAAGVTAMGTALSPSGMGAMGAQQAASMNGLGPYAAAMN |
| | | PCMSPMAYAPSNLGRSRAGGGGDAKTFKRSYPHAKPPYSYISLIT |
| | | MAIQQAPSKMLTLSEIYQWIMDLFPYYRQNQQRWQNSIRHSLSEN |
| | | DCFVKVARSPDKPGKGSYWTLHPDSGNMFENGCYLRRQKRFKCEK |
| | | QPGAGGGGGSGSGGSGAKGGPESRKDPSGASNPSADSPLHRGVHG |
| | | KTGQLEGAPAPGPAASPQTLDHSGATATGGASELKTPASSTAPPI |
| | | SSGPGALASVPASHPAHGLAPHESQLHLKGDPHYSFNHPFSINNL |
| | | MSSSEQQHKLDFKAYEQALQYSPYGSTLPASLPLGSASVTTRSPI |
| | | EPSALEPAYYQGVYSRPVLNTS |
| 103 | FOXA2 (HNF3B C- | MLGAVKMEGHEPSDWSSYYAEPEGYSSVSNMNAGLGMNGMNTYMS |
| | terminal domain) | MSAAAMGSGSGNMSAGSMNMSSYVGAGMSPSLAGMSPGAGAMAGM |
| | | GGSAGAAGVAGMGPHLSPSLSPLGGQAAGAMGGLAPYANMNSMSP |
| | | MYGQAGLSRARDPKTYRRSYTHAKPPYSYISLITMAIQQSPNKML |
| | | TLSEIYQWIMDLFPFYRQNQQRWQNSIRHSLSENDCFLKVPRSPD |
| | | KPGKGSFWTLHPDSGNMFENGCYLRRQKRFKCEKQLALKEAAGAA |
| | | GSGKKAAAGAQASQAQLGEAAGPASETPAGTESPHSSASPCQEHK |
| | | RGGLGELKGTPAAALSPPEPAPSPGQQQQAAAHLLGPPHHPGLPP |
| | | EAHLKPEHHYAFNHPFSINNLMSSEQQHHHSHHHHQPHKMDLKAY |
| | | EQVMHYPGYGSPMPGSLAMGPVINKTGLDASPLAADTSYYQGVYS |
| | | RPIMNSS |
| 104 | IRF2BP1 (IRF- | MASVQASRRQWCYLCDLPKMPWAMVWDFSEAVCRGCVNFEGADRI |
| | 2BP1_2 N-terminal | ELLIDAARQLKRSHVLPEGRSPGPPALKHPATKDLAAAAAQGPQL |
| | domain) | PPPQAQPQPSGTGGGVSGQDRYDRATSSGRLPLPSPALEYTLGSR |
| | | LANGLGREEAVAEGARRALLGSMPGLMPPGLLAAAVSGLGSRGLI |
| | | LAPGLSPARPLFGSDFEKEKQQRNADCLAELNEAMRGRAEEWHGR |
| | | PKAVREQLLALSACAPFNVRFKKDHGLVGRVFAFDATARPPGYEF |
| | | ELKLFTEYPCGSGNVYAGVLAVARQMFHDALREPGKALASSGFKY |
| | | LEYERRHGSGEWRQLGELLTDGVRSFREPAPAEALPQQYPEPAPA |
| | | ALCGPPPRAPSRNLAPTPRRRKASPEPEGEAAGKMITEEQQQRHW |
| | | VAPGGPYSAETPGVPSPIAALKNVAEALGHSPKDPGGGGGPVRAG |
| | | GASPAASSTAQPPTQHRLVARNGEAEVSPTAGAEAVSGGGSGTGA |
| | | TPGAPLCCTLCRERLEDTHFVQCPSVPGHKFCFPCSREFIKAQGP |
| | | AGEVYCPSGDKCPLVGSSVPWAFMQGEIATILAGDIKVKKERDP |
| 105 | IRF2BP2 (IRF- | MAAAVAVAAASRRQSCYLCDLPRMPWAMIWDFTEPVCRGCVNYEG |
| | 2BP1_2 N-terminal | ADRVEFVIETARQLKRAHGCFPEGRSPPGAAASAAAKPPPLSAKD |
| | domain) | ILLQQQQQLGHGGPEAAPRAPQALERYPLAAAAERPPRLGSDFGS |
| | | SRPAASLAQPPTPQPPPVNGILVPNGFSKLEEPPELNRQSPNPRR |
| | | GHAVPPTLVPLMNGSATPLPTALGLGGRAAASLAAVSGTAAASLG |
| | | SAQPTDLGAHKRPASVSSSAAVEHEQREAAAKEKQPPPPAHRGPA |
| | | DSLSTAAGAAELSAEGAGKSRGSGEQDWVNRPKTVRDILLALHQH |
| | | GHSGPFESKFKKEPALTAGRLLGFEANGANGSKAVARTARKRKPS |
| | | PEPEGEVGPPKINGEAQPWLSTSTEGLKIPMTPTSSFVSPPPPTA |
| | | SPHSNRITPPEAAQNGQSPMAALILVADNAGGSHASKDANQVHST |
| | | TRRNSNSPPSPSSMNQRRLGPREVGGQGAGNTGGLEPVHPASLPD |
| | | SSLATSAPLCCTLCHERLEDTHFVQCPSVPSHKFCFPCSRQSIKQ |
| | | QGASGEVYCPSGEKCPLVGSNVPWAFMQGEIATILAGDVKVKKER |
| | | DS |
| 106 | IRF2BPL IRF- | MSAAQVSSSRRQSCYLCDLPRMPWAMIWDFSEPVCRGCVNYEGAD |
| | 2BP1_2 N-terminal | RIEFVIETARQLKRAHGCFQDGRSPGPPPPVGVKTVALSAKEAAA |
| | domain | AAAAAAAAAAAAQQQQQQQQQQQQQQQQQQQQQQQQQLNHVDGSS |
| | | KPAVLAAPSGLERYGLSAAAAAAAAAAAAVEQRSRFEYPPPPVSL |
| | | GSSSHTARLPNGLGGPNGFPKPTPEEGPPELNRQSPNSSSAAASV |
| | | ASRRGTHGGLVTGLPNPGGGGGPQLTVPPNLLPQTLLNGPASAAV |
| | | LPPPPPHALGSRGPPTPAPPGAPGGPACLGGTPGVSATSSSASSS |
| | | TSSSVAEVGVGAGGKRPGSVSSTDQERELKEKQRNAEALAELSES |
| | | LRNRAEEWASKPKMVRDTLLTLAGCTPYEVRFKKDHSLLGRVFAF |
| | | DAVSKPGMDYELKLFIEYPTGSGNVYSSASGVAKQMYQDCMKDFG |
| | | RGLSSGFKYLEYEKKHGSGDWRLLGDLLPEAVRFFKEGVPGADML |
| | | PQPYLDASCPMLPTALVSLSRAPSAPPGTGALPPAAPSGRGAAAS |
| | | LRKRKASPEPPDSAEGALKLGEEQQRQQWMANQSEALKLTMSAGG |
| | | FAAPGHAAGGPPPPPPPLGPHSNRTTPPESAPQNGPSPMAALMSV |
| | | ADTLGTAHSPKDGSSVHSTTASARRNSSSPVSPASVPGQRRLASR |
| | | NGDLNLQVAPPPPSAHPGMDQVHPQNIPDSPMANSGPLCCTICHE |
| | | RLEDTHFVQCPSVPSHKFCFPCSRESIKAQGATGEVYCPSGEKCP |
| | | LVGSNVPWAFMQGEIATILAGDVKVKKERDP |
| 107 | HOXA13 | MTASVLLHPRWIEPTVMFLYDNGGGLVADELNKNMEGAAAAAAAA |
| | (homeodomain) | AAAAAAGAGGGGFPHPAAAAAGGNFSVAAAAAAAAAAAANQCRNL |
| | | MAHPAPLAPGAASAYSSAPGEAPPSAAAAAAAAAAAAAAAAAASS |
| | | SGGPGPAGPAGAEAAKQCSPCSAAAQSSSGPAALPYGYFGSGYYP |
| | | CARMGPHPNAIKSCAQPASAAAAAAFADKYMDTAGPAAEEFSSRA |
| | | KEFAFYHQGYAAGPYHHHQPMPGYLDMPVVPGLGGPGESRHEPLG |
| | | LPMESYQPWALPNGWNGQMYCPKEQAQPPHLWKSTLPDVVSHPSD |
| | | ASSYRRGRKKRVPYTKVQLKELEREYATNKFITKDKRRRISATTN |
| | | LSERQVTIWFQNRRVKEKKVINKLKTTS |
| 108 | HOXB13 | MEPGNYATLDGAKDIEGLLGAGGGRNLVAHSPLISHPAAPTLMPA |
| | (homeodomain) | VNYAPLDLPGSAEPPKQCHPCPGVPQGTSPAPVPYGYFGGGYYSC |
| | | RVSRSSLKPCAQAATLAAYPAETPTAGEEYPSRPTEFAFYPGYPG |
| | | TYQPMASYLDVSVVQTLGAPGEPRHDSLLPVDSYQSWALAGGWNS |
| | | QMCCQGEQNPPGPFWKAAFADSSGQHPPDACAFRRGRKKRIPYSK |
| | | GQLRELEREYAANKFITKDKRRKISAATSLSERQITIWFQNRRVK |
| | | EKKVLAKVKNSATP |
| 109 | HOXC13 | MTTSLLLHPRWPESLMYVYEDSAAESGIGGGGGGGGGGTGGAGGG |
| | (homeodomain) | CSGASPGKAPSMDGLGSSCPASHCRDLLPHPVLGRPPAPLGAPQG |
| | | AVYTDIPAPEAARQCAPPPAPPTSSSATLGYGYPFGGSYYGCRLS |
| | | HNVNLQQKPCAYHPGDKYPEPSGALPGDDLSSRAKEFAFYPSFAS |
| | | SYQAMPGYLDVSVVPGISGHPEPRHDALIPVEGYQHWALSNGWDS |
| | | QVYCSKEQSQSAHLWKSPFPDVVPLQPEVSSYRRGRKKRVPYTKV |
| | | QLKELEKEYAASKFITKEKRRRISATTNLSERQVTIWFQNRRVKE |
| | | KKVVSKSKAPHLHST |
| 110 | HOXA11 | MDFDERGPCSSNMYLPSCTYYVSGPDFSSLPSFLPQTPSSRPMTY |
| | (homeodomain) | SYSSNLPQVQPVREVTFREYAIEPATKWHPRGNLAHCYSAEELVH |
| | | RDCLQAPSAAGVPGDVLAKSSANVYHHPTPAVSSNFYSTVGRNGV |
| | | LPQAFDQFFETAYGTPENLASSDYPGDKSAEKGPPAATATSAAAA |
| | | AAATGAPATSSSDSGGGGGCRETAAAAEEKERRRRPESSSSPESS |
| | | SGHTEDKAGGSSGQRTRKKRCPYTKYQIRELEREFFFSVYINKEK |
| | | RLQLSRMLNLTDRQVKIWFQNRRMKEKKINRDRLQYYSANPLL |
| 111 | HOXC11 | MFNSVNLGNFCSPSRKERGADFGERGSCASNLYLPSCTYYMPEFS |
| | (homeodomain) | TVSSFLPQAPSRQISYPYSAQVPPVREVSYGLEPSGKWHHRNSYS |
| | | SCYAAADELMHRECLPPSTVTEILMKNEGSYGGHHHPSAPHATPA |
| | | GFYSSVNKNSVLPQAFDRFFDNAYCGGGDPPAEPPCSGKGEAKGE |
| | | PEAPPASGLASRAEAGAEAEAEEENINPSSSGSAHSVAKEPAKGA |
| | | APNAPRTRKKRCPYSKFQIRELEREFFENVYINKEKRLQLSRMLN |
| | | LTDRQVKIWFQNRRMKEKKLSRDRLQYFSGNPLL |
| 112 | HOXC10 | MTCPRNVTPNSYAEPLAAPGGGERYSRSAGMYMQSGSDENCGVMR |
| | (homeodomain) | GCGLAPSLSKRDEGSSPSLALNTYPSYLSQLDSWGDPKAAYRLEQ |
| | | PVGRPLSSCSYPPSVKEENVCCMYSAEKRAKSGPEAALYSHPLPE |
| | | SCLGEHEVPVPSYYRASPSYSALDKTPHCSGANDFEAPFEQRASL |
| | | NPRAEHLESPQLGGKVSFPETPKSDSQTPSPNEIKTEQSLAGPKG |
| | | SPSESEKERAKAADSSPDTSDNEAKEEIKAENTTGNWLTAKSGRK |
| | | KRCPYTKHQTLELEKEFLFNMYLTRERRLEISKTINLTDRQVKIW |
| | | FQNRRMKLKKMNRENRIRELTSNENFT |
| 113 | HOXA10 | MSARKGYLLPSPNYPTTMSCSESPAANSFLVDSLISSGRGEAGGG |
| | (homeodomain) | GGGAGGGGGGGYYAHGGVYLPPAADLPYGLQSCGLFPTLGGKRNE |
| | | AASPGSGGGGGGLGPGAHGYGPSPIDLWLDAPRSCRMEPPDGPPP |
| | | PPQQQPPPPPQPPQPAPQATSCSFAQNIKEESSYCLYDSADKCPK |
| | | VSATAAELAPFPRGPPPDGCALGTSSGVPVPGYFRLSQAYGTAKG |
| | | YGSGGGGAQQLGAGPFPAQPPGRGFDLPPALASGSADAARKERAL |
| | | DSPPPPTLACGSGGGSQGDEEAHASSSAAEELSPAPSESSKASPE |
| | | KDSLGNSKGENAANWLTAKSGRKKRCPYTKHQTLELEKEFLENMY |
| | | LTRERRLEISRSVHLTDRQVKIWFQNRRMKLKKMNRENRIRELTA |
| | | NENFS |
| 114 | HOXB9 | MSISGTLSSYYVDSIISHESEDAPPAKFPSGQYASSRQPGHAEHL |
| | (homeodomain) | EFPSCSFQPKAPVEGASWAPLSPHASGSLPSVYHPYIQPQGVPPA |
| | | ESRYLRTWLEPAPRGEAAPGQGQAAVKAEPLLGAPGELLKQGTPE |
| | | YSLETSAGREAVLSNQRPGYGDNKICEGSEDKERPDQTNPSANWL |
| | | HARSSRKKRCPYTKYQTLELEKEFLENMYLTRDRRHEVARLLNLS |
| | | ERQVKIWFQNRRMKMKKMNKEQGKE |
| 115 | HOXA9 | MATTGALGNYYVDSFLLGADAADELSVGRYAPGTLGQPPRQAATL |
| | (homeodomain) | AEHPDFSPCSFQSKATVEGASWNPVHAAGANAVPAAVYHHHHHHP |
| | | YVHPQAPVAAAAPDGRYMRSWLEPTPGALSFAGLPSSRPYGIKPE |
| | | PLSARRGDCPTLDTHTLSLTDYACGSPPVDREKQPSEGAFSENNA |
| | | ENESGGDKPPIDPNNPAANWLHARSTRKKRCPYTKHQTLELEKEF |
| | | LFNMYLTRDRRYEVARLLNLTERQVKIWFQNRRMKMKKINKDRAK |
| | | DE |
| 116 | ZFP28_HUMAN | NKKLEAVGTGIEPKAMSQGLVTFGDVAVDFSQEEWEWLNPIQRNL |
| | | YRKVMLENYRNLASLGLCVSKPDVISSLEQGKEPW |
| 117 | ZN334_HUMAN | KMKKFQIPVSFQDLTVNFTQEEWQQLDPAQRLLYRDVMLENYSNL |
| | | VSVGYHVSKPDVIFKLEQGEEPWIVEEFSNQNYPD |
| 118 | ZN568_HUMAN | CSQESALSEEEEDTTRPLETVTFKDVAVDLTQEEWEQMKPAQRNL |
| | | YRDVMLENYSNLVTVGCQVTKPDVIFKLEQEEEPW |
| 119 | ZN37A_HUMAN | ITSQGSVSFRDVTVGFTQEEWQHLDPAQRTLYRDVMLENYSHLVS |
| | | VGYCIPKPEVILKLEKGEEPWILEEKFPSQSHLEL |
| 120 | ZN181_HUMAN | PQVTFNDVAIDFTHEEWGWLSSAQRDLYKDVMVQNYENLVSVAGL |
| | | SVTKPYVITLLEDGKEPWMMEKKLSKGMIPDWESR |
| 121 | ZN510_HUMAN | PLRFSTLFQEQQKMNISQASVSFKDVTIEFTQEEWQQMAPVQKNL |
| | | YRDVMLENYSNLVSVGYCCFKPEVIFKLEQGEEPW |
| 122 | ZN862_HUMAN | QDPSAEGLSEEVPVVFEELPVVFEDVAVYFTREEWGMLDKRQKEL |
| | | YRDVMRMNYELLASLGPAAAKPDLISKLERRAAPW |
| 123 | ZN140_HUMAN | SQGSVTFRDVAIDFSQEEWKWLQPAQRDLYRCVMLENYGHLVSLG |
| | | LSISKPDVVSLLEQGKEPWLGKREVKRDLFSVSES |
| 124 | ZN208_HUMAN | GSLTFRDVAIEFSLEEWQCLDTAQQNLYRNVMLENYRNLVFLGIA |
| | | AFKPDLIIFLEEGKESWNMKRHEMVEESPVICSHF |
| 125 | ZN248_HUMAN | NKSQEQVSFKDVCVDFTQEEWYLLDPAQKILYRDVILENYSNLVS |
| | | VGYCITKPEVIFKIEQGEEPWILEKGFPSQCHPER |
| 126 | ZN571_HUMAN | PHLLVTFRDVAIDFSQEEWECLDPAQRDLYRDVMLENYSNLISLD |
| | | LESSCVTKKLSPEKEIYEMESLQWENMGKRINHHL |
| 127 | ZN699_HUMAN | EEERKTAELQKNRIQDSVVFEDVAVDFTQEEWALLDLAQRNLYRD |
| | | VMLENFQNLASLGYPLHTPHLISQWEQEEDLQTVK |
| 128 | ZN726_HUMAN | GLLTFRDVAIEFSLEEWQCLDTAQKNLYRNVMLENYRNLAFLGIA |
| | | VSKPDLIICLEKEKEPWNMKRDEMVDEPPGICPHF |
| 129 | ZIK1_HUMAN | RAPTQVTVSPETHMDLTKGCVTFEDIAIYFSQDEWGLLDEAQRLL |
| | | YLEVMLENFALVASLGCGHGTEDEETPSDQNVSVG |
| 130 | ZNF2_HUMAN | AAVSPTTRCQESVTFEDVAVVFTDEEWSRLVPIQRDLYKEVMLEN |
| | | YNSIVSLGLPVPQPDVIFQLKRGDKPWMVDLHGSE |
| 131 | Z705F_HUMAN | HSLEKVTFEDVAIDFTQEEWDMMDTSKRKLYRDVMLENISHLVSL |
| | | GYQISKSYIILQLEQGKELWREGRVFLQDQNPDRE |
| 132 | ZNF14_HUMAN | DSVSFEDVAVNFTLEEWALLDSSQKKLYEDVMQETFKNLVCLGKK |
| | | WEDQDIEDDHRNQGKNRRCHMVERLCESRRGSKCG |
| 133 | ZN471_HUMAN | NVEVVKVMPQDLVTFKDVAIDFSQEEWQWMNPAQKRLYRSMMLEN |
| | | YQSLVSLGLCISKPYVISLLEQGREPWEMTSEMTR |
| 134 | ZN624_HUMAN | TQPDEDLHLQAEETQLVKESVTFKDVAIDFTLEEWRLMDPTQRNL |
| | | HKDVMLENYRNLVSLGLAVSKPDMISHLENGKGPW |
| 135 | ZNF84_HUMAN | TMLQESFSFDDLSVDFTQKEWQLLDPSQKNLYKDVMLENYSSLVS |
| | | LGYEVMKPDVIFKLEQGEEPWVGDGEIPSSDSPEV |
| 136 | ZNF7_HUMAN | EVVTFGDVAVHFSREEWQCLDPGQRALYREVMLENHSSVAGLAGF |
| | | LVFKPELISRLEQGEEPWVLDLQGAEGTEAPRTSK |
| 137 | ZN891_HUMAN | RNAEEERMIAVFLTTWLQEPMTFKDVAVEFTQEEWMMLDSAQRSL |
| | | YRDVMLENYRNLISVEYQLYRLTVISPLDQEEIRN |
| 138 | ZN337_HUMAN | GPQGARRQAFLAFGDVTVDFTQKEWRLLSPAQRALYREVTLENYS |
| | | HLVSLGILHSKPELIRRLEQGEVPWGEERRRRPGP |
| 139 | Z705G_HUMAN | HSLKKLTFEDVAIDFTQEEWAMMDTSKRKLYRDVMLENISHLVSL |
| | | GYQISKSYIILQLEQGKELWREGRVFLQDQNPNRE |
| 140 | ZN529_HUMAN | MPEVEFPDQFFTVLTMDHELVTLRDVVINFSQEEWEYLDSAQRNL |
| | | YWDVMMENYSNLLSLDLESRNETKHLSVGKDIIQN |
| 141 | ZN729_HUMAN | PGAPGSLEMGPLTFRDVTIEFSLEEWQCLDTVQQNLYRDVMLENY |
| | | RNLVFLGMAVFKPDLITCLKQGKEPWNMKRHEMVT |
| 142 | ZN419_HUMAN | RDPAQVPVAADLLTDHEEGYVTFEDVAVYFSQEEWRLLDDAQRLL |
| | | YRNVMLENFTLLASLGLASSKTHEITQLESWEEPF |
| 143 | Z705A_HUMAN | HSLKKVTFEDVAIDFTQEEWAMMDTSKRKLYRDVMLENISHLVSL |
| | | GYQISKSYIILQLEQGKELWREGREFLQDQNPDRE |
| 144 | ZNF45_HUMAN | TKSKEAVTFKDVAVVFSEEELQLLDLAQRKLYRDVMLENFRNVVS |
| | | VGHQSTPDGLPQLEREEKLWMMKMATQRDNSSGAK |
| 145 | ZN302_HUMAN | SQVTFSDVAIDFSHEEWACLDSAQRDLYKDVMVQNYENLVSVGLS |
| | | VTKPYVIMLLEDGKEPWMMEKKLSKAYPFPLSHSV |
| 146 | ZN486_HUMAN | PGPLRSLEMESLQFRDVAVEFSLEEWHCLDTAQQNLYRDVMLENY |
| | | RHLVFLGIIVSKPDLITCLEQGIKPLTMKRHEMIA |
| 147 | ZN621_HUMAN | LQTTWPQESVTFEDVAVYFTQNQWASLDPAQRALYGEVMLENYAN |
| | | VASLVAFPFPKPALISHLERGEAPWGPDPWDTEIL |
| 148 | ZN688_HUMAN | APLLAPRPGETRPGCRKPGTVSFADVAVYFSPEEWGCLRPAQRAL |
| | | YRDVMQETYGHLGALGFPGPKPALISWMEQESEAW |
| 149 | ZN33A_HUMAN | NKVEQKSQESVSFKDVTVGFTQEEWQHLDPSQRALYRDVMLENYS |
| | | NLVSVGYCVHKPEVIFRLQQGEEPWKQEEEFPSQS |
| 150 | ZN554_HUMAN | CFSQEERMAAGYLPRWSQELVTFEDVSMDFSQEEWELLEPAQKNL |
| | | YREVMLENYRNVVSLEALKNQCTDVGIKEGPLSPA |
| 151 | ZN878_HUMAN | DSVAFEDVAVNFTQEEWALLDPSQKNLYREVMQETLRNLTSIGKK |
| | | WNNQYIEDEHQNPRRNLRRLIGERLSESKESHQHG |
| 152 | ZN772_HUMAN | MGPAQVPMNSEVIVDPIQGQVNFEDVFVYFSQEEWVLLDEAQRLL |
| | | YRDVMLENFALMASLGHTSFMSHIVASLVMGSEPW |
| 153 | ZN224_HUMAN | TTFKEAMTFKDVAVVFTEEELGLLDLAQRKLYRDVMLENFRNLLS |
| | | VGHQAFHRDTFHFLREEKIWMMKTAIQREGNSGDK |
| 154 | ZN184_HUMAN | DSTLLQGGHNLLSSASFQEAVTFKDVIVDETQEEWKQLDPGQRDL |
| | | FRDVTLENYTHLVSIGLQVSKPDVISQLEQGTEPW |
| 155 | ZN544_HUMAN | EARSMLVPPQASVCFEDVAMAFTQEEWEQLDLAQRTLYREVTLET |
| | | WEHIVSLGLFLSKSDVISQLEQEEDLCRAEQEAPR |
| 156 | ZNF57_HUMAN | DSVVFEDVAVDFTLEEWALLDSAQRDLYRDVMLETFRNLASVDDG |
| | | TQFKANGSVSLQDMYGQEKSKEQTIPNFTGNNSCA |
| 157 | ZN283_HUMAN | EESHGALISSCNSRIMTDGLVTFRDVAIDFSQEEWECLDPAQRDL |
| | | YVDVMLENYSNLVSLDLESKTYETKKIFSENDIFE |
| 158 | ZN549_HUMAN | VITPQIPMVTEEFVKPSQGHVTFEDIAVYFSQEEWGLLDEAQRCL |
| | | YHDVMLENFSLMASVGCLHGIEAEEAPSEQTLSAQ |
| 159 | ZN211_HUMAN | VQLRPQTRMATALRDPASGSVTFEDVAVYFSWEEWDLLDEAQKHL |
| | | YFDVMLENFALTSSLGCWCGVEHEETPSEQRISGE |
| 160 | ZN615_HUMAN | MQAQESLTLEDVAVDFTWEEWQFLSPAQKDLYRDVMLENYSNLVA |
| | | VGYQASKPDALSKLERGEETCTTEDEIYSRICSEI |
| 161 | ZN253_HUMAN | GPLQFRDVAIEFSLEEWHCLDTAQRNLYRDVMLENYRNLVFLGIV |
| | | VSKPDLVTCLEQGKKPLTMERHEMIAKPPVMSSHF |
| 162 | ZN226_HUMAN | NMFKEAVTFKDVAVAFTEEELGLLGPAQRKLYRDVMVENFRNLLS |
| | | VGHPPFKQDVSPIERNEQLWIMTTATRRQGNLGEK |
| 163 | ZN730_HUMAN | GALTFRDVAIEFSLEEWQCLDTEQQNLYRNVMLDNYRNLVFLGIA |
| | | VSKPDLITCLEQEKEPWNLKTHDMVAKPPVICSHI |
| 164 | Z585A_HUMAN | SPQKSSALAPEDHGSSYEGSVSFRDVAIDFSREEWRHLDPSQRNL |
| | | YRDVMLETYSHLLSVGYQVPEAEVVMLEQGKEPWA |
| 165 | ZN732_HUMAN | ELLTFRDVAIEFSPEEWKCLDPAQQNLYRDVMLENYRNLISLGVA |
| | | ISNPDLVIYLEQRKEPYKVKIHETVAKHPAVCSHF |
| 166 | ZN681_HUMAN | EPLKFRDVAIEFSLEEWQCLDTIQQNLYRNVMLENYRNLVFLGIV |
| | | VSKPDLITCLEQEKEPWTRKRHRMVAEPPVICSHF |
| 167 | ZN667_HUMAN | PSARGKSKSKAPITFGDLAIYFSQEEWEWLSPIQKDLYEDVMLEN |
| | | YRNLVSLGLSFRRPNVITLLEKGKAPWMVEPVRRR |
| 168 | ZN649_HUMAN | TKAQESLTLEDVAVDFTWEEWQFLSPAQKDLYRDVMLENYSNLVS |
| | | VGYQAGKPDALTKLEQGEPLWTLEDEIHSPAHPEI |
| 169 | ZN470_HUMAN | SQEEVEVAGIKLCKAMSLGSVTFTDVAIDFSQDEWEWLNLAQRSL |
| | | YKKVMLENYRNLVSVGLCISKPDVISLLEQEKDPW |
| 170 | ZN484_HUMAN | TKSLESVSFKDVTVDFSRDEWQQLDLAQKSLYREVMLENYFNLIS |
| | | VGCQVPKPEVIFSLEQEEPCMLDGEIPSQSRPDGD |
| 171 | ZN431_HUMAN | SGCPGAERNLLVYSYFEKETLTFRDVAIEFSLEEWECLNPAQQNL |
| | | YMNVMLENYKNLVFLGVAVSKQDPVTCLEQEKEPW |
| 172 | ZN382_HUMAN | PLQGSVSFKDVTVDFTQEEWQQLDPAQKALYRDVMLENYCHFVSV |
| | | GFHMAKPDMIRKLEQGEELWTQRIFPSYSYLEEDG |
| 173 | ZN254_HUMAN | PGPPRSLEMGLLTFRDVAIEFSLEEWQHLDIAQQNLYRNVMLENY |
| | | RNLAFLGIAVSKPDLITCLEQGKEPWNMKRHEMVD |
| 174 | ZN124_HUMAN | SGHPGSWEMNSVAFEDVAVNFTQEEWALLDPSQKNLYRDVMQETF |
| | | RNLASIGNKGEDQSIEDQYKNSSRNLRHIISHSGN |
| 175 | ZN607_HUMAN | SYGSITFGDVAIDFSHQEWEYLSLVQKTLYQEVMMENYDNLVSLA |
| | | GHSVSKPDLITLLEQGKEPWMIVREETRGECTDLD |
| 176 | ZN317_HUMAN | DLFVCSGLEPHTPSVGSQESVTFQDVAVDFTEKEWPLLDSSQRKL |
| | | YKDVMLENYSNLTSLGYQVGKPSLISHLEQEEEPR |
| 177 | ZN620_HUMAN | FQTAWRQEPVTFEDVAVYFTQNEWASLDSVQRALYREVMLENYAN |
| | | VASLAFPFTTPVLVSQLEQGELPWGLDPWEPMGRE |
| 178 | ZN141 HUMAN | ELLTFRDVAIEFSPEEWKCLDPDQQNLYRDVMLENYRNLVSLGVA |
| | | ISNPDLVTCLEQRKEPYNVKIHKIVARPPAMCSHF |
| 179 | ZN584_HUMAN | AGEAEAQLDPSLQGLVMFEDVTVYFSREEWGLLNVTQKGLYRDVM |
| | | LENFALVSSLGLAPSRSPVFTQLEDDEQSWVPSWV |
| 180 | ZN540_HUMAN | AHALVTFRDVAIDFSQKEWECLDTTQRKLYRDVMLENYNNLVSLG |
| | | YSGSKPDVITLLEQGKEPCVVARDVTGRQCPGLLS |
| 181 | ZN75D_HUMAN | KRIKHWKMASKLILPESLSLLIFEDVAVYFSEEEWQLLNPLEKTL |
| | | YNDVMQDIYETVISLGLKLKNDTGNDHPISVSTSE |
| 182 | ZN555_HUMAN | DSVVFEDVAVDFTLEEWALLDSAQRDLYRDVMLETFQNLASVDDE |
| | | TQFKASGSVSQQDIYGEKIPKESKIATFTRNVSWA |
| 183 | ZN658_HUMAN | NMSQASVSFQDVTVEFTREEWQHLGPVERTLYRDVMLENYSHLIS |
| | | VGYCITKPKVISKLEKGEEPWSLEDEFLNQRYPGY |
| 184 | ZN684_HUMAN | ISFQESVTFQDVAVDFTAEEWQLLDCAERTLYWDVMLENYRNLIS |
| | | VGCPITKTKVILKVEQGQEPWMVEGANPHESSPES |
| 185 | RBAK_HUMAN | NTLQGPVSFKDVAVDFTQEEWQQLDPDEKITYRDVMLENYSHLVS |
| | | VGYDTTKPNVIIKLEQGEEPWIMGGEFPCQHSPEA |
| 186 | ZN829_HUMAN | HPEEEERMHDELLQAVSKGPVMFRDVSIDFSQEEWECLDADQMNL |
| | | YKEVMLENFSNLVSVGLSNSKPAVISLLEQGKEPW |
| 187 | ZN582_HUMAN | SLGSELFRDVAIVFSQEEWQWLAPAQRDLYRDVMLETYSNLVSLG |
| | | LAVSKPDVISFLEQGKEPWMVERVVSGGLCPVLES |
| 188 | ZN112_HUMAN | TKFQEMVTFKDVAVVFTEEELGLLDSVQRKLYRDVMLENFRNLLL |
| | | VAHQPFKPDLISQLEREEKLLMVETETPRDGCSGR |
| 189 | ZN716_HUMAN | AKRPGPPGSREMGLLTFRDIAIEFSLAEWQCLDHAQQNLYRDVML |
| | | ENYRNLVSLGIAVSKPDLITCLEQNKEPQNIKRNE |
| 190 | HKR1_HUMAN | TCMVHRQTMSCSGAGGITAFVAFRDVAVYFTQEEWRLLSPAQRTL |
| | | HREVMLETYNHLVSLEIPSSKPKLIAQLERGEAPW |
| 191 | ZN350_HUMAN | IQAQESITLEDVAVDFTWEEWQLLGAAQKDLYRDVMLENYSNLVA |
| | | VGYQASKPDALFKLEQGEQLWTIEDGIHSGACSDI |
| 192 | ZN480_HUMAN | AQKRRKRKAKESGMALPQGHLTFRDVAIEFSQAEWKCLDPAQRAL |
| | | YKDVMLENYRNLVSLGISLPDLNINSMLEQRREPW |
| 193 | ZN416_HUMAN | DSTSVPVTAEAKLMGFTQGCVTFEDVAIYFSQEEWGLLDEAQRLL |
| | | YRDVMLENFALITALVCWHGMEDEETPEQSVSVEG |
| 194 | ZNF92_HUMAN | GPLTFRDVKIEFSLEEWQCLDTAQRNLYRDVMLENYRNLVFLGIA |
| | | VSKPDLITWLEQGKEPWNLKRHEMVDKTPVMCSHF |
| 195 | ZN100_HUMAN | SGCPGAERSLLVQSYFEKGPLTFRDVAIEFSLEEWQCLDSAQQGL |
| | | YRKVMLENYRNLVFLAGIALTKPDLITCLEQGKEP |
| 196 | ZN736_HUMAN | GVLTFRDVAVEFSPEEWECLDSAQQRLYRDVMLENYGNLVSLGLA |
| | | IFKPDLMTCLEQRKEPWKVKRQEAVAKHPAGSFHF |
| 197 | ZNF74_HUMAN | KENLEDISGWGLPEARSKESVSFKDVAVDFTQEEWGQLDSPQRAL |
| | | YRDVMLENYQNLLALGPPLHKPDVISHLERGEEPW |
| 198 | CBX1_HUMAN | EESEKPRGFARGLEPERIIGATDSSGELMFLMKWKNSDEADLVPA |
| | | KEANVKCPQVVISFYEERLTWHSYPSEDDDKKDDK |
| 199 | ZN443_HUMAN | ASVALEDVAVNFTREEWALLGPCQKNLYKDVMQETIRNLDCVVMK |
| | | WKDQNIEDQYRYPRKNLRCRMLERFVESKDGTQCG |
| 200 | ZN195_HUMAN | TLLTFRDVAIEFSLEEWKCLDLAQQNLYRDVMLENYRNLFSVGLT |
| | | VCKPGLITCLEQRKEPWNVKRQEAADGHPEMGFHH |
| 201 | ZN530_HUMAN | AAALRAPTQQVFVAFEDVAIYFSQEEWELLDEMQRLLYRDVMLEN |
| | | FAVMASLGCWCGAVDEGTPSAESVSVEELSQGRIP |
| 202 | ZN782_HUMAN | NTFQASVSFQDVTVEFSQEEWQHMGPVERTLYRDVMLENYSHLVS |
| | | VGYCFTKPELIFTLEQGEDPWLLEKEKGFLSRNSP |
| 203 | ZN791_HUMAN | DSVAFEDVSVSFSQEEWALLAPSQKKLYRDVMQETFKNLASIGEK |
| | | WEDPNVEDQHKNQGRNLRSHIGERLCEGKEGSQCA |
| 204 | ZN331_HUMAN | AQGLVTFADVAIDFSQEEWACLNSAQRDLYWDVMLENYSNLVSLD |
| | | LESAYENKSLPTEKNIHEIRASKRNSDRRSKSLGR |
| 205 | Z354C_HUMAN | AVDLLSAQEPVTFRDVAVFFSQDEWLHLDSAQRALYREVMLENYS |
| | | SLVSLGIPFSMPKLIHQLQQGEDPCMVEREVPSDT |
| 206 | ZN157_HUMAN | SPQRFPALIPGEPGRSFEGSVSFEDVAVDFTRQEWHRLDPAQRTM |
| | | HKDVMLETYSNLASVGLCVAKPEMIFKLERGEELW |
| 207 | ZN727_HUMAN | RVLTFRDVAVEFSPEEWECLDSAQQRLYRDVMLENYGNLFSLGLA |
| | | IFKPDLITYLEQRKEPWNARRQKTVAKHPAGSLHF |
| 208 | ZN550_HUMAN | AETKDAAQMLVTFKDVAVTFTREEWRQLDLAQRTLYREVMLETCG |
| | | LLVSLGHRVPKPELVHLLEHGQELWIVKRGLSHAT |
| 209 | ZN793_HUMAN | IEYQIPVSFKDVVVGFTQEEWHRLSPAQRALYRDVMLETYSNLVS |
| | | VGYEGTKPDVILRLEQEEAPWIGEAACPGCHCWED |
| 210 | ZN235_HUMAN | TKFQEAVTFKDVAVAFTEEELGLLDSAQRKLYRDVMLENFRNLVS |
| | | VGHQSFKPDMISQLEREEKLWMKELQTQRGKHSGD |
| 211 | ZNF8_HUMAN | DEGVAGVMSVGPPAARLQEPVTFRDVAVDFTQEEWGQLDPTQRIL |
| | | YRDVMLETFGHLLSIGPELPKPEVISQLEQGTELW |
| 212 | ZN724_HUMAN | GPLTFMDVAIEFSVEEWQCLDTAQQNLYRNVMLENYRNLVFLGIA |
| | | VSKPDLITCLEQGKEPWNMERHEMVAKPPGMCCYF |
| 213 | ZN573_HUMAN | HQVGLIRSYNSKTMTCFQELVTFRDVAIDFSRQEWEYLDPNQRDL |
| | | YRDVMLENYRNLVSLGGHSISKPVVVDLLERGKEP |
| 214 | ZN577_HUMAN | NATIVMSVRREQGSSSGEGSLSFEDVAVGFTREEWQFLDQSQKVL |
| | | YKEVMLENYINLVSIGYRGTKPDSLFKLEQGEPPG |
| 215 | ZN789_HUMAN | FPPARGKELLSFEDVAMYFTREEWGHLNWGQKDLYRDVMLENYRN |
| | | MVLLGFQFPKPEMICQLENWDEQWILDLPRTGNRK |
| 216 | ZN718_HUMAN | ELLTFKDVAIEFSPEEWKCLDTSQQNLYRDVMLENYRNLVSLGVS |
| | | ISNPDLVTSLEQRKEPYNLKIHETAARPPAVCSHF |
| 217 | ZN300_HUMAN | MKSQGLVSFKDVAVDFTQEEWQQLDPSQRTLYRDVMLENYSHLVS |
| | | MGYPVSKPDVISKLEQGEEPWIIKGDISNWIYPDE |
| 218 | ZN383_HUMAN | AEGSVMFSDVSIDFSQEEWDCLDPVQRDLYRDVMLENYGNLVSMG |
| | | LYTPKPQVISLLEQGKEPWMVGRELTRGLCSDLES |
| 219 | ZN429_HUMAN | GPLTFTDVAIEFSLEEWQCLDTAQQNLYRNVMLENYRNLVFLGIA |
| | | VSKPDLITCLEKEKEPCKMKRHEMVDEPPVVCSHF |
| 220 | ZN677_HUMAN | ALSQGLFTFKDVAIEFSQEEWECLDPAQRALYRDVMLENYRNLLS |
| | | LDEDNIPPEDDISVGFTSKGLSPKENNKEELYHLV |
| 221 | ZN850_HUMAN | NMEGLVMFQDLSIDFSQEEWECLDAAQKDLYRDVMMENYSSLVSL |
| | | GLSIPKPDVISLLEQGKEPWMVSRDVLGGWCRDSE |
| 222 | ZN454_HUMAN | AVSHLPTMVQESVTFKDVAILFTQEEWGQLSPAQRALYRDVMLEN |
| | | YSNLVSLGLLGPKPDTFSQLEKREVWMPEDTPGGF |
| 223 | ZN257_HUMAN | GPLTIRDVTVEFSLEEWHCLDTAQQNLYRDVMLENYRNLVFLGIA |
| | | VSKPDLITCLEQGKEPCNMKRHEMVAKPPVMCSHI |
| 224 | ZN264_HUMAN | AAAVLTDRAQVSVTFDDVAVTFTKEEWGQLDLAQRTLYQEVMLEN |
| | | CGLLVSLGCPVPKAELICHLEHGQEPWTRKEDLSQ |
| 225 | ZFP82_HUMAN | ALRSVMFSDVSIDFSPEEWEYLDLEQKDLYRDVMLENYSNLVSLG |
| | | CFISKPDVISSLEQGKEPWKVVRKGRRQYPDLETK |
| 226 | ZFP14_HUMAN | AHGSVTFRDVAIDFSQEEWEFLDPAQRDLYRDVMWENYSNFISLG |
| | | PSISKPDVITLLDEERKEPGMVVREGTRRYCPDLE |
| 227 | ZN485_HUMAN | APRAQIQGPLTFGDVAVAFTRIEWRHLDAAQRALYRDVMLENYGN |
| | | LVSVGLLSSKPKLITQLEQGAEPWTEVREAPSGTH |
| 228 | ZN737_HUMAN | GPLQFRDVAIEFSLEEWHCLDTAQRNLYRNVMLENYRNLVFLGIV |
| | | VSKPDLITCLEQGKKPLTMKKHEMVANPSVTCSHF |
| 229 | ZNF44_HUMAN | TLPRGQPEVLEWGLPKDQDSVAFEDVAVNFTHEEWALLGPSQKNL |
| | | YRDVMRETIRNLNCIGMKWENQNIDDQHQNLRRNP |
| 230 | ZN596_HUMAN | PSPDSMTFEDIIVDFTQEEWALLDTSQRKLFQDVMLENISHLVSI |
| | | GKQLCKSVVLSQLEQVEKLSTQRISLLQGREVGIK |
| 231 | ZN565_HUMAN | EESREIRAGQIVLKAMAQGLVTFRDVAIEFSLEEWKCLEPAQRDL |
| | | YREVTLENFGHLASLGLSISKPDVVSLLEQGKEPW |
| 232 | ZN543_HUMAN | AASAQVSVTFEDVAVTFTQEEWGQLDAAQRTLYQEVMLETCGLLM |
| | | SLGCPLFKPELIYQLDHRQELWMATKDLSQSSYPG |
| 233 | ZFP69_HUMAN | RESLEDEVTPGLPTAESQELLTFKDISIDFTQEEWGQLAPAHQNL |
| | | YREVMLENYSNLVSVGYQLSKPSVISQLEKGEEPW |
| 234 | SUMO1_HUMAN | EGEYIKLKVIGQDSSEIHFKVKMTTHLKKLKESYCQRQGVPMNSL |
| | | RFLFEGQRIADNHTPKELGMEEEDVIEVYQEQTGG |
| 235 | ZNF12_HUMAN | NKSLGPVSFKDVAVDETQEEWQQLDPEQKITYRDVMLENYSNLVS |
| | | VGYHIIKPDVISKLEQGEEPWIVEGEFLLQSYPDE |
| 236 | ZN169_HUMAN | SPGLLTTRKEALMAFRDVAVAFTQKEWKLLSSAQRTLYREVMLEN |
| | | YSHLVSLGIAFSKPKLIEQLEQGDEPWREENEHLL |
| 237 | ZN433_HUMAN | MFQDSVAFEDVAVTFTQEEWALLDPSQKNLCRDVMQETFRNLASI |
| | | GKKWKPQNIYVEYENLRRNLRIVGERLFESKEGHQ |
| 238 | SUMO3_HUMAN | ENDHINLKVAGQDGSVVQFKIKRHTPLSKLMKAYCERQGLSMRQI |
| | | RFRFDGQPINETDTPAQLEMEDEDTIDVEQQQTGG |
| 239 | ZNF98_HUMAN | PGPLGSLEMGVLTFRDVALEFSLEEWQCLDTAQQNLYRNVMLENY |
| | | RNLVFVGIAASKPDLITCLEQGKEPWNVKRHEMVT |
| 240 | ZN175_HUMAN | LSQKPQVLGPEKQDGSCEASVSFEDVTVDFSREEWQQLDPAQRCL |
| | | YRDVMLELYSHLFAVGYHIPNPEVIFRMLKEKEPR |
| 241 | ZN347_HUMAN | ALTQGQVTFRDVAIEFSQEEWTCLDPAQRTLYRDVMLENYRNLAS |
| | | LGISCFDLSIISMLEQGKEPFTLESQVQIAGNPDG |
| 242 | ZNF25_HUMAN | NKFQGPVTLKDVIVEFTKEEWKLLTPAQRTLYKDVMLENYSHLVS |
| | | VGYHVNKPNAVFKLKQGKEPWILEVEFPHRGFPED |
| 243 | ZN519_HUMAN | ELLTFRDVAIEFSPEEWKCLDPAQQNLYRDVMLENYRNLVSLAVY |
| | | SYYNQGILPEQGIQDSFKKATLGRYGSCGLENICL |
| 244 | Z585B_HUMAN | SPQKSSALAPEDHGSSYEGSVSFRDVAIDFSREEWRHLDLSQRNL |
| | | YRDVMLETYSHLLSVGYQVPKPEVVMLEQGKEPWA |
| 245 | ZIM3_HUMAN | NNSQGRVTFEDVTVNFTQGEWQRLNPEQRNLYRDVMLENYSNLVS |
| | | VGQGETTKPDVILRLEQGKEPWLEEEEVLGSGRAE |
| 246 | ZN517_HUMAN | AMALPMPGPQEAVVFEDVAVYFTRIEWSCLAPDQQALYRDVMLEN |
| | | YGNLASLGFLVAKPALISLLEQGEEPGALILQVAE |
| 247 | ZN846_HUMAN | DSSQHLVTFEDVAVDFTQEEWTLLDQAQRDLYRDVMLENYKNLII |
| | | LAGSELFKRSLMSGLEQMEELRTGVTGVLQELDLQ |
| 248 | ZN230_HUMAN | TTFKEAVTFKDVAVFFTEEELGLLDPAQRKLYQDVMLENFINLLS |
| | | VGHQPFHPFHFLREEKFWMMETATQREGNSGGKTI |
| 249 | ZNF66_HUMAN | GPLQFRDVAIEFSLEEWHCLDMAQRNLYRDVMLENYRNLVFLGIV |
| | | VSKPDLITHLEQGKKPSTMQRHEMVANPSVLCSHF |
| 250 | ZFP1_HUMAN | NKSQGSVSFTDVTVDFTQEEWEQLDPSQRILYMDVMLENYSNLLS |
| | | VEVWKADDQMERDHRNPDEQARQFLILKNQTPIEE |
| 251 | ZN713_HUMAN | EEEEMNDGSQMVRSQESLTFQDVAVDFTREEWDQLYPAQKNLYRD |
| | | VMLENYRNLVALGYQLCKPEVIAQLELEEEWVIER |
| 252 | ZN816_HUMAN | EEATKKSKEKEPGMALPQGRLTFRDVAIEFSLEEWKCLNPAQRAL |
| | | YRAVMLENYRNLEFVDSSLKSMMEFSSTRHSITGE |
| 253 | ZN426_HUMAN | EKTPAGRIVADCLTDCYQDSVTFDDVAVDFTQEEWILLDSTQRSL |
| | | YSDVMLENYKNLATVGGQIIKPSLISWLEQEESRT |
| 254 | ZN674_HUMAN | AMSQESLTFKDVFVDFTLEEWQQLDSAQKNLYRDVMLENYSHLVS |
| | | VGHLVGKPDVIFRLGPGDESWMADGGTPVRTCAGE |
| 255 | ZN627_HUMAN | DSVAFEDVAVNFTLEEWALLDPSQKNLYRDVMRETFRNLASVGKQ |
| | | WEDQNIEDPFKIPRRNISHIPERLCESKEGGQGEE |
| 256 | ZNF20_HUMAN | MFQDSVAFEDVAVSFTQEEWALLDPSQKNLYRDVMQETFKNLTSV |
| | | GKTWKVQNIEDEYKNPRRNLSLMREKLCESKESHH |
| 257 | Z587B_HUMAN | AVVATLRLSAQGTVTFEDVAVKFTQEEWNLLSEAQRCLYRDVTLE |
| | | NLALMSSLGCWCGVEDEAAPSKQSIYIQRETQVRT |
| 258 | ZN316_HUMAN | EEEEEDEDEDDLLTAGCQELVTFEDVAVYFSLEEWERLEADQRGL |
| | | YQEVMQENYGILVSLGYPIPKPDLIFRLEQGEEPW |
| 259 | ZN233_HUMAN | TKFQEMVTFKDVAVVFTREELGLLDLAQRKLYQDVMLENFRNLLS |
| | | VGYQPFKLDVILQLGKEDKLRMMETEIQGDGCSGH |
| 260 | ZN611_HUMAN | EEAAQKRKGKEPGMALPQGRLTFRDVAIEFSLAEWKCLNPSQRAL |
| | | YREVMLENYRNLEAVDISSKCMMKEVLSTGQGNTE |
| 261 | ZN556_HUMAN | DTVVFEDVVVDFTLEEWALLNPAQRKLYRDVMLETFKHLASVDNE |
| | | AQLKASGSISQQDTSGEKLSLKQKIEKFTRKNIWA |
| 262 | ZN234_HUMAN | TTFKEGLTFKDVAVVFTEEELGLLDPVQRNLYQDVMLENERNLLS |
| | | VGHHPFKHDVFLLEKEKKLDIMKTATQRKGKSADK |
| 263 | ZN560_HUMAN | SALQQEFWKIQTSNGIQMDLVTFDSVAVEFTQEEWILLDPAQRNL |
| | | YSDVMLENYKNLSSVGYQLFKPSLISWLEEEEELS |
| 264 | ZNF77_HUMAN | DCVIFEEVAVNETPEEWALLDHAQRSLYRDVMLETCRNLASLDCY |
| | | IYVRTSGSSSQRDVFGNGISNDEEIVKFTGSDSWS |
| 265 | ZN682_HUMAN | ELLTFRDVTIEFSLEEWEFLNPAQQSLYRKVMLENYRNLVSLGLT |
| | | VSKPELISRLEQRQEPWNVKRHETIAKPPAMSSHY |
| 266 | ZN614_HUMAN | IKTQESLTLEDVAVEFSWEEWQLLDTAQKNLYRDVMVENYNHLVS |
| | | LGYQTSKPDVLSKLAHGQEPWITDAKIQNKNCPGI |
| 267 | ZN785_HUMAN | PAHVPGEAGPRRTRESRPGAVSFADVAVYFSPEEWECLRPAQRAL |
| | | YRDVMRETFGHLGALGFSVPKPAFISWVEGEVEAW |
| 268 | ZN445_HUMAN | GCPGDQVTPTRSLTAQLQETMTFKDVEVTFSQDEWGWLDSAQRNL |
| | | YRDVMLENYRNMASLVGPFTKPALISWLEAREPWG |
| 269 | ZFP30_HUMAN | ARDLVMFRDVAVDFSQEEWECLNSYQRNLYRDVILENYSNLVSLA |
| | | GCSISKPDVITLLEQGKEPWMVVRDEKRRWILDLE |
| 270 | ZN225_HUMAN | TTLKEAVTFKDVAVVFTEEELRLLDLAQRKLYREVMLENFRNLLS |
| | | VGHQSLHRDTFHFLKEEKFWMMETATQREGNLGGK |
| 271 | ZN551_HUMAN | SPPSPRSSMAAVALRDSAQGMTFEDVAIYFSQEEWELLDESQRFL |
| | | YCDVMLENFAHVTSLGYCHGMENEAIASEQSVSIQ |
| 272 | ZN610_HUMAN | DEEAQKRKAKESGMALPQGRLTFMDVAIEFSQEEWKSLDPGQRAL |
| | | YRDVMLENYRNLVFLGICLPDLSIISMLKQRREPL |
| 273 | ZN528_HUMAN | ALTQGPLKFMDVAIEFSQEEWKCLDPAQRTLYRDVMLENYRNLVS |
| | | LGICLPDLSVTSMLEQKRDPWTLQSEEKIANDPDG |
| 274 | ZN284_HUMAN | TMFKEAVTFKDVAVVFTEEELGLLDVSQRKLYRDVMLENFRNLLS |
| | | VGHQLSHRDTFHFQREEKFWIMETATQREGNSGGK |
| 275 | ZN418_HUMAN | QGTVAFEDVAVNFSQEEWSLLSEVQRCLYHDVMLENWVLISSLGC |
| | | WCGSEDEEAPSKKSISIQRVSQVSTPGAGVSPKKA |
| 276 | MPP8_HUMAN | AEAFGDSEEDGEDVFEVEKILDMKTEGGKVLYKVRWKGYTSDDDT |
| | | WEPEIHLEDCKEVLLEFRKKIAENKAKAVRKDIQR |
| 277 | ZN490_HUMAN | VLQMQNSEHHGQSIKTQTDSISLEDVAVNFTLEEWALLDPGQRNI |
| | | YRDVMRATFKNLACIGEKWKDQDIEDEHKNQGRNL |
| 278 | ZN805_HUMAN | AMALTDPAQVSVTFDDVAVTFTQEEWGQLDLAQRTLYQEVMLENC |
| | | GLLVSLGCPVPRPELIYHLEHGQEPWIRKEDLSQG |
| 279 | Z780B_HUMAN | VHGSVTFRDVAIDFSQEEWECLQPDQRTLYRDVMLENYSHLISLG |
| | | SSISKPDVITLLEQEKEPWIVVSKETSRWYPDLES |
| 280 | ZN763_HUMAN | DPVACEDVAVNFTQEEWALLDISQRKLYREVMLETFRNLTSIGKK |
| | | WKDQNIEYEYQNPRRNFRSLIEGNVNEIKEDSHCG |
| 281 | ZN285_HUMAN | IKFQERVTFKDVAVVFTKEELALLDKAQINLYQDVMLENFRNLML |
| | | VRDGIKNNILNLQAKGLSYLSQEVLHCWQIWKQRI |
| 282 | ZNF85_HUMAN | GPLTFRDVAIEFSLKEWQCLDTAQRNLYRNVMLENYRNLVFLGIT |
| | | VSKPDLITCLEQGKEAWSMKRHEIMVAKPTVMCSH |
| 283 | ZN223_HUMAN | TMSKEAVTFKDVAVVFTEEELGLLDLAQRKLYRDVMLENFRNLLS |
| | | VGHQPFHRDTFHFLREEKFWMMDIATQREGNSGGK |
| 284 | ZNF90_HUMAN | GPLEFRDVAIEFSLEEWHCLDTAQQNLYRDVMLENYRHLVFLGIV |
| | | VTKPDLITCLEQGKKPFTVKRHEMIAKSPVMCFHF |
| 285 | ZN557_HUMAN | GHTEGGELVNELLKSWLKGLVTFEDVAVEFTQEEWALLDPAQRTL |
| | | YRDVMLENCRNLASLGNQVDKPRLISQLEQEDKVM |
| 286 | ZN425_HUMAN | AEPASVTVTEDDVALYFSEQEWEILEKWQKQMYKQEMKINYETLD |
| | | SLGYAFSKPDLITWMEQGRMLLISEQGCLDKTRRT |
| 287 | ZN229_HUMAN | HSQASAISQDREEKIMSQEPLSFKDVAVVFTEEELELLDSTQRQL |
| | | YQDVMQENFRNLLSVGERNPLGDKNGKDTEYIQDE |
| 288 | ZN606_HUMAN | GSLEEGRRATGLPAAQVQEPVTFKDVAVDFTQEEWGQLDLVQRTL |
| | | YRDVMLETYGHLLSVGNQIAKPEVISLLEQGEEPW |
| 289 | ZN155_HUMAN | TTFKEAVTFKDVAVVFTEEELGLLDPAQRKLYRDVMLENFRNLLS |
| | | VGHQPFHQDTCHFLREEKFWMMGTATQREGNSGGK |
| 290 | ZN222_HUMAN | AKLYEAVTFKDVAVIFTEEELGLLDPAQRKLYRDVMLENFRNLLS |
| | | VGGKIQTEMETVPEAGTHEEFSCKQIWEQIASDLI |
| 291 | ZN442_HUMAN | RSDLFLPDSQTNEERKQYDSVAFEDVAVNFTQEEWALLGPSQKSL |
| | | YRDVMWETIRNLDCIGMKWEDTNIEDQHRNPRRSL |
| 292 | ZNF91_HUMAN | PGTPGSLEMGLLTFRDVAIEFSPEEWQCLDTAQQNLYRNVMLENY |
| | | RNLAFLGIALSKPDLITYLEQGKEPWNMKQHEMVD |
| 293 | ZN135_HUMAN | TPGVRVSTDPEQVTFEDVVVGFSQEEWGQLKPAQRTLYRDVMLDT |
| | | FRLLVSVGHWLPKPNVISLLEQEAELWAVESRLPQ |
| 294 | ZN778_HUMAN | EQTQAAGMVAGWLINCYQDAVTEDDVAVDFTQEEWTLLDPSQRDL |
| | | YRDVMLENYENLASVEWRLKTKGPALRQDRSWFRA |
| 295 | RYBP_HUMAN | PSEANSIQSANATTKTSETNHTSRPRLKNVDRSTAQQLAVTVGNV |
| | | TVIITDFKEKTRSSSTSSSTVTSSAGSEQQNQSSS |
| 296 | ZN534_HUMAN | ALTQGQLSFSDVAIEFSQEEWKCLDPGQKALYRDVMLENYRNLVS |
| | | LGEDNVRPEACICSGICLPDLSVTSMLEQKRDPWT |
| 297 | ZN586_HUMAN | AAAAALRAPAQSSVTFEDVAVNFSLEEWSLLNEAQRCLYRDVMLE |
| | | TLTLISSLGCWHGGEDEAAPSKQSTCIHIYKDQGG |
| 298 | ZN567_HUMAN | AQGSVSFNDVTVDFTQEEWQHLDHAQKTLYMDVMLENYCHLISVG |
| | | CHMTKPDVILKLERGEEPWTSFAGHTCLEENWKAE |
| 299 | ZN440_HUMAN | DPVAFKDVAVNFTQEEWALLDISQRKLYREVMLETFRNLTSLGKR |
| | | WKDQNIEYEHQNPRRNFRSLIEEKVNEIKDDSHCG |
| 300 | ZN583_HUMAN | SKDLVTFGDVAVNFSQEEWEWLNPAQRNLYRKVMLENYRSLVSLG |
| | | VSVSKPDVISLLEQGKEPWMVKKEGTRGPCPDWEY |
| 301 | ZN441_HUMAN | DSVAFEDVAINFTCEEWALLGPSQKSLYRDVMQETIRNLDCIGMI |
| | | WQNHDIEEDQYKDLRRNLRCHMVERACEIKDNSQC |
| 302 | ZNF43_HUMAN | GPLTFMDVAIEFCLEEWQCLDIAQQNLYRNVMLENYRNLVFLGIA |
| | | VSKPDLITCLEQEKEPWEPMRRHEMVAKPPVMCSH |
| 303 | CBX5_HUMAN | QSNDIARGFERGLEPEKIIGATDSCGDLMFLMKWKDTDEADLVLA |
| | | KEANVKCPQIVIAFYEERLTWHAYPEDAENKEKET |
| 304 | ZN589_HUMAN | ALPAKDSAWPWEEKPRYLGPVTFEDVAVLFTEAEWKRLSLEQRNL |
| | | YKEVMLENLRNLVSLAESKPEVHTCPSCPLAFGSQ |
| 305 | ZNF10_HUMAN | DAKSLTAWSRTLVTFKDVFVDFTREEWKLLDTAQQIVYRNVMLEN |
| | | YKNLVSLGYQLTKPDVILRLEKGEEPWLVEREIHQ |
| 306 | ZN563_HUMAN | DAVAFEDVAVNFTQEEWALLGPSQKNLYRYVMQETIRNLDCIRMI |
| | | WEEQNTEDQYKNPRRNLRCHMVERFSESKDSSQCG |
| 307 | ZN561_HUMAN | EKTKVERMVEDYLASGYQDSVTFDDVAVDFTPEEWALLDITEKYL |
| | | YRDVMLENYMNLASVEWEIQPRTKRSSLQQGFLKN |
| 308 | ZN136_HUMAN | DSVAFEDVDVNFTQEEWALLDPSQKNLYRDVMWETMRNLASIGKK |
| | | WKDQNIKDHYKHRGRNLRSHMLERLYQTKDGSQRG |
| 309 | ZN630_HUMAN | IESQEPVTFEDVAVDFTQEEWQQLNPAQKTLHRDVMLETYNHLVS |
| | | VGCSGIKPDVIFKLEHGKDPWIIESELSRWIYPDR |
| 310 | ZN527_HUMAN | AVGLCKAMSQGLVTFRDVALDFSQEEWEWLKPSQKDLYRDVMLEN |
| | | YRNLVWLGLSISKPNMISLLEQGKEPWMVERKMSQ |
| 311 | ZN333_HUMAN | DKVEEEAMAPGLPTACSQEPVTFADVAVVFTPEEWVELDSTQRSL |
| | | YRDVMLENYRNLASVADQLCKPNALSYLEERGEQW |
| 312 | Z324B_HUMAN | TFEDVAVYFSQEEWGLLDTAQRALYRHVMLENFTLVTSLGLSTSR |
| | | PRVVIQLERGEEPWVPSGKDMTLARNTYGRLNSGS |
| 313 | ZN786_HUMAN | AEPPRLPLTFEDVAIYFSEQEWQDLEAWQKELYKHVMRSNYETLV |
| | | SLDDGLPKPELISWIEHGGEPFRKWRESQKSGNII |
| 314 | ZN709_HUMAN | DSVVFEDVAVNFTQEEWALLGPSQKKLYRDVMQETFVNLASIGEN |
| | | WEEKNIEDHKNQGRKLRSHMVERLCERKEGSQFGE |
| 315 | ZN792_HUMAN | AAAALRDPAQGCVTFEDVTIYFSQEEWVLLDEAQRLLYCDVMLEN |
| | | FALIASLGLISFRSHIVSQLEMGKEPWVPDSVDMT |
| 316 | ZN599_HUMAN | AAPALALVSFEDVVVTFTGEEWGHLDLAQRTLYQEVMLETCRLLV |
| | | SLGHPVPKPELIYLLEHGQELWTVKRGLSQSTCAG |
| 317 | ZN613_HUMAN | IKSQESLTLEDVAVEFTWEEWQLLGPAQKDLYRDVMLENYSNLVS |
| | | VGYQASKPDALFKLEQGEPWTVENEIHSQICPEIK |
| 318 | ZF69B_HUMAN | GESLESRVTLGSLTAESQELLTFKDVSVDFTQEEWGQLAPAHRNL |
| | | YREVMLENYGNLVSVGCQLSKPGVISQLEKGEEPW |
| 319 | ZN799_HUMAN | ASVALEDVAVNFTREEWALLGPCQKNLYKDVMQETIRNLDCVGMK |
| | | WKDQNIEDQYRYPRKNLRCRMLERFVESKDGTQCG |
| 320 | ZN569_HUMAN | TESQGTVTFKDVAIDFTQEEWKRLDPAQRKLYRNVMLENYNNLIT |
| | | VGYPFTKPDVIFKLEQEEEPWVMEEEVLRRHWQGE |
| 321 | ZN564_HUMAN | DSVASEDVAVNFTLEEWALLDPSQKKLYRDVMRETFRNLACVGKK |
| | | WEDQSIEDWYKNQGRILRNHMEEGLSESKEYDQCG |
| 322 | ZN546_HUMAN | EETQGELTSSCGSKTMANVSLAFRDVSIDLSQEEWECLDAVQRDL |
| | | YKDVMLENYSNLVSLGYTIPKPDVITLLEQEKEPW |
| 323 | ZFP92_HUMAN | AAILLTTRPKVPVSFEDVSVYFTKTEWKLLDLRQKVLYKRVMLEN |
| | | YSHLVSLGFSFSKPHLISQLERGEGPWVADIPRTW |
| 324 | YAF2_HUMAN | KDKVEKEKSEKETTSKKNSHKKTRPRLKNVDRSSAQHLEVTVGDL |
| | | TVIITDFKEKTKSPPASSAASADQHSQSGSSSDNT |
| 325 | ZN723_HUMAN | GPLTFTDVAIKFSLEEWQFLDTAQQNLYRDVMLENYRNLVFLGVG |
| | | VSKPDLITCLEQGKEPWNMKRHKMVAKPPVVCSHF |
| 326 | ZNF34_HUMAN | RKPNPQAMAALFLSAPPQAEVTFEDVAVYLSREEWGRLGPAQRGL |
| | | YRDVMLETYGNLVSLGVGPAGPKPGVISQLERGDE |
| 327 | ZN439_HUMAN | LSLSPILLYTCEMFQDPVAFKDVAVNFTQEEWALLDISQKNLYRE |
| | | VMLETFWNLTSIGKKWKDQNIEYEYQNPRRNFRSV |
| 328 | ZFP57_HUMAN | AAGEPRSLLFFQKPVTFEDVAVNFTQEEWDCLDASQRVLYQDVMS |
| | | ETFKNLISVARIFLHKPELITKLEQEEEQWRETRV |
| 329 | ZNF19_HUMAN | AAMPLKAQYQEMVTFEDVAVHFTKTEWTGLSPAQRALYRSVMLEN |
| | | FGNLTALGYPVPKPALISLLERGDMAWGLEAQDDP |
| 330 | ZN404_HUMAN | ARVPLTFSDVAIDFSQEEWEYLNSDQRDLYRDVMLENYTNLVSLD |
| | | FNFTTESNKLSSEKRNYEVNAYHQETWKRNKTENL |
| 331 | ZN274_HUMAN | ASRLPTAWSCEPVTFEDVTLGFTPEEWGLLDLKQKSLYREVMLEN |
| | | YRNLVSVEHQLSKPDVVSQLEEAEDFWPVERGIPQ |
| 332 | CBX3_HUMAN | SKKKRDAADKPRGFARGLDPERIIGATDSSGELMFLMKWKDSDEA |
| | | DLVLAKEANMKCPQIVIAFYEERLTWHSCPEDEAQ |
| 333 | ZNF30_HUMAN | AHKYVGLQYHGSVTFEDVAIAFSQQEWESLDSSQRGLYRDVMLEN |
| | | YRNLVSMGHSRSKPHVIALLEQWKEPEVTVRKDGR |
| 334 | ZN250_HUMAN | AAARLLPVPAGPQPLSFQAKLIFEDVAVLLSQDEWDRLCPAQRGL |
| | | YRNVMMETYGNVVSLGLPGSKPDIISQLERGEDPW |
| 335 | ZN570_HUMAN | AVGLLKAMYQELVTFRDVAVDFSQEEWDCLDSSQRHLYSNVMLEN |
| | | YRILVSLGLCFSKPSVILLLEQGKAPWMVKRELTK |
| 336 | ZN675_HUMAN | GLLTFRDVAIEFSLEEWQCLDTAQRNLYKNVILENYRNLVFLGIA |
| | | VSKQDLITCLEQEKEPLTVKRHEMVNEPPVMCSHF |
| 337 | ZN695_HUMAN | GLLAFRDVALEFSPEEWECLDPAQRSLYRDVMLENYRNLISLGED |
| | | SFNMQFLFHSLAMSKPELIICLEARKEPWNVNTEK |
| 338 | ZN548_HUMAN | NLTEGRVVFEDVAIYFSQEEWGHLDEAQRLLYRDVMLENLALLSS |
| | | LGSWHGAEDEEAPSQQGFSVGVSEVTASKPCLSSQ |
| 339 | ZN132_HUMAN | GPAQHTSWPCGSAVPTLKSMVTFEDVAVYFSQEEWELLDAAQRHL |
| | | YHSVMLENLELVTSLGSWHGVEGEGAHPKQNVSVE |
| 340 | ZN738_HUMAN | SGYPGAERNLLEYSYFEKGPLTFRDVVIEFSQEEWQCLDTAQQDL |
| | | YRKVMLENFRNLVFLGIDVSKPDLITCLEQGKDPW |
| 341 | ZN420_HUMAN | ARKLVMFRDVAIDFSQEEWECLDSAQRDLYRDVMLENYSNLVSLD |
| | | LPSRCASKDLSPEKNTYETELSQWEMSDRLENCDL |
| 342 | ZN626_HUMAN | GPLQFRDVAIEFSLEEWHCLDTAQRNLYRNVMLENYSNLVFLGIT |
| | | VSKPDLITCLEQGRKPLTMKRNEMIAKPSVMCSHF |
| 343 | ZN559_HUMAN | VAGWLTNYSQDSVTFEDVAVDFTQEEWILLDQTQRNLYRDVMLEN |
| | | YKNLVAVDWESHINTKWSAPQQNFLQGKTSSVVEM |
| 344 | ZN460_HUMAN | AAAWMAPAQESVTFEDVAVTFTQEEWGQLDVTQRALYVEVMLETC |
| | | GLLVALGDSTKPETVEPIPSHLALPEEVSLQEQLA |
| 345 | ZN268_HUMAN | VLEWLFISQEQPKITKSWGPLSFMDVFVDFTWEEWQLLDPAQKCL |
| | | YRSVMLENYSNLVSLGYQHTKPDIIFKLEQGEELC |
| 346 | ZN304_HUMAN | AAAVLMDRVQSCVTFEDVFVYFSREEWELLEEAQRFLYRDVMLEN |
| | | FALVATLGFWCEAEHEAPSEQSVSVEGVSQVRTAE |
| 347 | ZIM2_HUMAN | AGSQFPDFKHLGTFLVFEELVTFEDVLVDFSPEELSSLSAAQRNL |
| | | YREVMLENYRNLVSLGHQFSKPDIISRLEEEESYA |
| 348 | ZN605_HUMAN | IQSQISFEDVAVDFTLEEWQLLNPTQKNLYRDVMLENYSNLVFLE |
| | | VWLDNPKMWLRDNQDNLKSMERGHKYDVFGKIENS |
| 349 | ZN844_HUMAN | DLVAFEDVAVNFTQEEWSLLDPSQKNLYREVMQETLRNLASIGEK |
| | | WKDQNIEDQYKNPRNNLRSLLGERVDENTEENHCG |
| 350 | SUMO5_HUMAN | KDEDIKLRVIGQDSSEIHFKVKMTTPLKKLKKSYCQRQGVPVNSL |
| | | RFLFEGQRIADNHTPEELGMEEEDVIEVYQEQIGG |
| 351 | ZN101_HUMAN | DSVAFEDVAVNFTQEEWALLSPSQKNLYRDVTLETFRNLASVGIQ |
| | | WKDQDIENLYQNLGIKLRSLVERLCGRKEGNEHRE |
| 352 | ZN783_HUMAN | RNFWILRLPPGSKGEAPKVPVTFDDVAVYFSELEWGKLEDWQKEL |
| | | YKHVMRGNYETLVSLDYAISKPDILTRIERGEEPC |
| 353 | ZN417_HUMAN | AAAAPRRPTQQGTVTFEDVAVNFSQEEWCLLSEAQRCLYRDVMLE |
| | | NLALISSLGCWCGSKDEEAPCKQRISVQRESQSRT |
| 354 | ZN182_HUMAN | SGEDSGSFYSWQKAKREQGLVTFEDVAVDFTQEEWQYLNPPQRTL |
| | | YRDVMLETYSNLVFVGQQVTKPNLILKLEVEECPA |
| 355 | ZN823_HUMAN | DSVAFEDVAVNFTQEEWALLGPSQKSLYRNVMQETIRNLDCIEMK |
| | | WEDQNIGDQCQNAKRNLRSHTCEIKDDSQCGETFG |
| 356 | ZN177_HUMAN | AAGWLTTWSQNSVTFQEVAVDFSQEEWALLDPAQKNLYKDVMLEN |
| | | FRNLASVGYQLCRHSLISKVDQEQLKTDERGILQG |
| 357 | ZN197_HUMAN | ENPRNQLMALMLLTAQPQELVMFEEVSVCFTSEEWACLGPIQRAL |
| | | YWDVMLENYGNVTSLEWETMTENEEVTSKPSSSQR |
| 358 | ZN717_HUMAN | LETYNSLVSLQELVSFEEVAVHFTWEEWQDLDDAQRTLYRDVMLE |
| | | TYSSLVSLGHCITKPEMIFKLEQGAEPWIVEETPN |
| 359 | ZN669_HUMAN | RHFRRPEPCREPLASPIQDSVAFEDVAVNFTQEEWALLDSSQKNL |
| | | YREVMQETCRNLASVGSQWKDQNIEDHFEKPGKDI |
| 360 | ZN256_HUMAN | AAAELTAPAQGIVTFEDVAVYFSWKEWGLLDEAQKCLYHDVMLEN |
| | | LTLTTSLGGSGAGDEEAPYQQSTSPQRVSQVRIPK |
| 361 | ZN251_HUMAN | AATFQLPGHQEMPLTFQDVAVYFSQAEGRQLGPQQRALYRDVMLE |
| | | NYGNVASLGFPVPKPELISQLEQGKELWVLNLLGA |
| 362 | CBX4_HUMAN | RSEAGEPPSSLQVKPETPASAAVAVAAAAAPTTTAEKPPAEAQDE |
| | | PAESLSEFKPFFGNIIITDVTANCLIVIFKEYVTV |
| 363 | PCGF2_HUMAN | HRTTRIKITELNPHIMCALCGGYFIDATTIVECLHSFCKTCIVRY |
| | | LETNKYCPMCDVQVHKTRPLLSIRSDKTLQDIVYK |
| 364 | CDY2_HUMAN | ASQEFEVEAIVDKRQDKNGNTQYLVRWKGYDKQDDTWEPEQHLMN |
| | | CEKCVHDENRRQTEKQKKLTWTTTSRIFSNNARRR |
| 365 | CDYL2_HUMAN | ASGDLYEVERIVDKRKNKKGKWEYLIRWKGYGSTEDTWEPEHHLL |
| | | HCEEFIDEFNGLHMSKDKRIKSGKQSSTSKLLRDS |
| 366 | HERC2_HUMAN | TLIRKADLENHNKDGGFWTVIDGKVYDIKDFQTQSLTGNSILAQF |
| | | AGEDPVVALEAALQFEDTRESMHAFCVGQYLEPDQ |
| 367 | ZN562_HUMAN | EKTKIGTMVEDHRSNSYQDSVTFDDVAVEFTPEEWALLDTTQKYL |
| | | YRDVMLENYMNLASVDFFFCLTSEWEIQPRTKRSS |
| 368 | ZN461_HUMAN | AHELVMERDVAIDVSQEEWECLNPAQRNLYKEVMLENYSNLVSLG |
| | | LSVSKPAVISSLEQGKEPWMVVREETGRWCPGTWK |
| 369 | Z324A_HUMAN | AFEDVAVYFSQEEWGLLDTAQRALYRRVMLDNFALVASLGLSTSR |
| | | PRVVIQLERGEEPWVPSGTDTTLSRTTYRRRNPGS |
| 370 | ZN766_HUMAN | AQLRRGHLTFRDVAIEFSQEEWKCLDPVQKALYRDVMLENYRNLV |
| | | SLGICLPDLSIISMMKQRTEPWTVENEMKVAKNPD |
| 371 | ID2_HUMAN | SDHSLGISRSKTPVDDPMSLLYNMNDCYSKLKELVPSIPQNKKVS |
| | | KMEILQHVIDYILDLQIALDSHPTIVSLHHQRPGQ |
| 372 | TOX_HUMAN | KDPNEPQKPVSAYALFFRDTQAAIKGQNPNATFGEVSKIVASMWD |
| | | GLGEEQKQVYKKKTEAAKKEYLKQLAAYRASLVSK |
| 373 | ZN274_HUMAN | QEEKQEDAAICPVTVLPEEPVTFQDVAVDFSREEWGLLGPTQRTE |
| | | YRDVMLETFGHLVSVGWETTLENKELAPNSDIPEE |
| 374 | SCMH1_HUMAN | DASRLSGRDPSSWTVEDVMQFVREADPQLGPHADLFRKHEIDGKA |
| | | LLLLRSDMMMKYMGLKLGPALKLSYHIDRLKQGKF |
| 375 | ZN214_HUMAN | AVTFEDVTIIFTWEEWKFLDSSQKRLYREVMWENYTNVMSVENWN |
| | | ESYKSQEEKFRYLEYENFSYWQGWWNAGAQMYENQ |
| 376 | CBX7_HUMAN | ELSAIGEQVFAVESIRKKRVRKGKVEYLVKWKGWPPKYSTWEPEE |
| | | HILDPRLVMAYEEKEERDRASGYRKRGPKPKRLLL |
| 377 | ID1_HUMAN | GGAGARLPALLDEQQVNVLLYDMNGCYSRLKELVPTLPQNRKVSK |
| | | VEILQHVIDYIRDLQLELNSESEVGIPGGRGLPVR |
| 378 | CREM_HUMAN | VVMAASPGSLHSPQQLAEEATRKRELRLMKNREAAKECRRRKKEY |
| | | VKCLESRVAVLEVQNKKLIEELETLKDICSPKTDY |
| 379 | SCX_HUMAN | GGGPGGRPGREPRQRHTANARERDRINSVNTAFTALRTLIPTEPA |
| | | DRKLSKIETLRLASSYISHLGNVLLAGEACGDGQP |
| 380 | ASCL1_HUMAN | SGFGYSLPQQQPAAVARRNERERNRVKLVNLGFATLREHVPNGAA |
| | | NKKMSKVETLRSAVEYIRALQQLLDEHDAVSAAFQ |
| 381 | ZN764_HUMAN | APLPPRDPNGAGPEWREPGAVSFADVAVYFCREEWGCLRPAQRAL |
| | | YRDVMRETYGHLSALGIGGNKPALISWVEEEAELW |
| 382 | SCML2_HUMAN | KQGFSKDPSTWSVDEVIQFMKHTDPQISGPLADLFRQHEIDGKAL |
| | | FLLKSDVMMKYMGLKLGPALKLCYYIEKLKEGKYS |
| 383 | TWST1_HUMAN | SGGGSPQSYEELQTQRVMANVRERQRTQSLNEAFAALRKIIPTLP |
| | | SDKLSKIQTLKLAARYIDFLYQVLQSDELDSKMAS |
| 384 | CREB1_HUMAN | IAPGVVMASSPALPTQPAEEAARKREVRLMKNREAARECRRKKKE |
| | | YVKCLENRVAVLENQNKTLIEELKALKDLYCHKSD |
| 385 | TERF1_HUMAN | SRIPVSKSQPVTPEKHRARKRQAWLWEEDKNLRSGVRKYGEGNWS |
| | | KILLHYKFNNRTSVMLKDRWRTMKKLKLISSDSED |
| 386 | ID3_HUMAN | SLAIARGRGKGPAAEEPLSLLDDMNHCYSRLRELVPGVPRGTQLS |
| | | QVEILQRVIDYILDLQVVLAEPAPGPPDGPHLPIQ |
| 387 | CBX8_HUMAN | GSGPPSSGGGLYRDMGAQGGRPSLIARIPVARILGDPEEESWSPS |
| | | LTNLEKVVVTDVTSNFLTVTIKESNTDQGFFKEKR |
| 388 | CBX4_HUMAN | ELPAVGEHVFAVESIEKKRIRKGRVEYLVKWRGWSPKYNTWEPEE |
| | | NILDPRLLIAFQNRERQEQLMGYRKRGPKPKPLVV |
| 389 | GSX1_HUMAN | VDSSSNQLPSSKRMRTAFTSTQLLELEREFASNMYLSRLRRIEIA |
| | | TYLNLSEKQVKIWFQNRRVKHKKEGKGSNHRGGGG |
| 390 | NKX22_HUMAN | TPGGGGDAGKKRKRRVLFSKAQTYELERRFRQQRYLSAPEREHLA |
| | | SLIRLIPTQVKIWFQNHRYKMKRARAEKGMEVTPL |
| 391 | ATF1_HUMAN | QTVVMTSPVTLISQTTKTDDPQLKREIRLMKNREAARECRRKKKE |
| | | YVKCLENRVAVLENQNKTLIEELKTLKDLYSNKSV |
| 392 | TWST2_HUMAN | KGSPSAQSFEELQSQRILANVRERQRTQSLNEAFAALRKIIPTLP |
| | | SDKLSKIQTLKLAARYIDFLYQVLQSDEMDNKMTS |
| 393 | ZNF17_HUMAN | NLTEDYMVFEDVAIHFSQEEWGILNDVQRHLHSDVMLENFALLSS |
| | | VGCWHGAKDEEAPSKQCVSVGVSQVTTLKPALSTQ |
| 394 | TOX3_HUMAN | KDPNEPQKPVSAYALFFRDTQAAIKGQNPNATFGEVSKIVASMWD |
| | | SLGEEQKQVYKRKTEAAKKEYLKALAAYRASLVSK |
| 395 | TOX4_HUMAN | KDPNEPQKPVSAYALFFRDTQAAIKGQNPNATFGEVSKIVASMWD |
| | | SLGEEQKQVYKRKTEAAKKEYLKALAAYKDNQECQ |
| 396 | ZMYM3_HUMAN | LDGSTWDFCSEDCKSKYLLWYCKAARCHACKRQGKLLETIHWRGQ |
| | | IRHFCNQQCLLRFYSQQNQPNLDTQSGPESLLNSQ |
| 397 | I2BP1_HUMAN | ASVQASRRQWCYLCDLPKMPWAMVWDFSEAVCRGCVNFEGADRIE |
| | | LLIDAARQLKRSHVLPEGRSPGPPALKHPATKDLA |
| 398 | RHXF1_HUMAN | MEGPQPENMQPRTRRTKFTLLQVEELESVFRHTQYPDVPTRRELA |
| | | ENLGVTEDKVRVWFKNKRARCRRHQRELMLANELR |
| 399 | SSX2_HUMAN | PKIMPKKPAEEGNDSEEVPEASGPQNDGKELCPPGKPTTSEKIHE |
| | | RSGPKRGEHAWTHRLRERKQLVIYEEISDPEEDDE |
| 400 | I2BPL_HUMAN | SAAQVSSSRRQSCYLCDLPRMPWAMIWDFSEPVCRGCVNYEGADR |
| | | IEFVIETARQLKRAHGCFQDGRSPGPPPPVGVKTV |
| 401 | ZN680_HUMAN | PGPPGSLEMGPLTFRDVAIEFSLEEWQCLDTAQRNLYRKVMFENY |
| | | RNLVFLGIAVSKPHLITCLEQGKEPWNRKRQEMVA |
| 402 | CBX1_HUMAN | NKKKVEEVLEEEEEEYVVEKVLDRRVVKGKVEYLLKWKGFSDEDN |
| | | TWEPEENLDCPDLIAEFLQSQKTAHETDKSEGGKR |
| 403 | TRI68_HUMAN | LANVVEKVRLLRLHPGMGLKGDLCERHGEKLKMFCKEDVLIMCEA |
| | | CSQSPEHEAHSVVPMEDVAWEYKWELHEALEHLKK |
| 404 | HXA13_HUMAN | VVSHPSDASSYRRGRKKRVPYTKVQLKELEREYATNKFITKDKRR |
| | | RISATTNLSERQVTIWFQNRRVKEKKVINKLKITS |
| 405 | PHC3_HUMAN | ENSDLLPVAQTEPSIWTVDDVWAFIHSLPGCQDIADEFRAQEIDG |
| | | QALLLLKEDHLMSAMNIKLGPALKICARINSLKES |
| 406 | TCF24_HUMAN | AGPGGGSRSGSGRPAAANAARERSRVQTLRHAFLELQRTLPSVPP |
| | | DTKLSKLDVLLLATTYIAHLTRSLQDDAEAPADAG |
| 407 | CBX3_HUMAN | QNGKSKKVEEAEPEEFVVEKVLDRRVVNGKVEYFLKWKGFTDADN |
| | | TWEPEENLDCPELIEAFLNSQKAGKEKDGTKRKSL |
| 408 | HXB13_HUMAN | QHPPDACAFRRGRKKRIPYSKGQLRELEREYAANKFITKDKRRKI |
| | | SAATSLSERQITIWFQNRRVKEKKVLAKVKNSATP |
| 409 | HEY1_HUMAN | SMSPTTSSQILARKRRRGIIEKRRRDRINNSLSELRRLVPSAFEK |
| | | QGSAKLEKAEILQMTVDHLKMLHTAGGKGYFDAHA |
| 410 | PHC2_HUMAN | LVGMGHHFLPSEPTKWNVEDVYEFIRSLPGCQEIAEEFRAQEIDG |
| | | QALLLLKEDHLMSAMNIKLGPALKIYARISMLKDS |
| 411 | ZNF81_HUMAN | PANEDAPQPGEHGSACEVSVSFEDVTVDFSREEWQQLDSTQRRLY |
| | | QDVMLENYSHLLSVGFEVPKPEVIFKLEQGEGPWT |
| 412 | FIGLA_HUMAN | GYSSTENLQLVLERRRVANAKERERIKNLNRGFARLKALVPFLPQ |
| | | SRKPSKVDILKGATEYIQVLSDLLEGAKDSKKQDP |
| 413 | SAM11_HUMAN | EEAPAPEDVTKWTVDDVCSFVGGLSGCGEYTRVFREQGIDGETLP |
| | | LLTEEHLLTNMGLKLGPALKIRAQVARRLGRVFYV |
| 414 | KMT2B_HUMAN | GGTLAHTPRRSLPSHHGKKMRMARCGHCRGCLRVQDCGSCVNCLD |
| | | KPKFGGPNTKKQCCVYRKCDKIEARKMERLAKKGR |
| 415 | HEY2_HUMAN | LNSPITTSQIMARKKRRGIIEKRRRDRINNSLSELRRLVPTAFEK |
| | | QGSAKLEKAEILQMTVDHLKMLQATGGKGYFDAHA |
| 416 | JDP2_HUMAN | QPVKSELDEEEERRKRRREKNKVAAARCRNKKKERTEFLQRESER |
| | | LELMNAELKTQIEELKQERQQLILMLNRHRPTCIV |
| 417 | HXC13_HUMAN | LQPEVSSYRRGRKKRVPYTKVQLKELEKEYAASKFITKEKRRRIS |
| | | ATTNLSERQVTIWFQNRRVKEKKVVSKSKAPHLHS |
| 418 | ASCLA_HUMAN | LPVPLDSAFEPAFLRKRNERERQRVRCVNEGYARLRDHLPRELAD |
| | | KRLSKVETLRAAIDYIKHLQELLERQAWGLEGAAG |
| 419 | HHEX_HUMAN | SPFLQRPLHKRKGGQVRFSNDQTIELEKKFETQKYLSPPERKRLA |
| | | KMLQLSERQVKTWFQNRRAKWRRLKQENPQSNKKE |
| 420 | HERC2_HUMAN | IAIATGSLHCVCCTEDGEVYTWGDNDEGQLGDGITTNAIQRPRLVA |
| | | ALQGKKVNRVACGSAHTLAWSTSKPASAGKLPAQV |
| 421 | GSX2_HUMAN | GGSDASQVPNGKRMRTAFTSTQLLELEREFSSNMYLSRLRRIEIA |
| | | TYLNLSEKQVKIWFQNRRVKHKKEGKGTQRNSHAG |
| 422 | BIN1_HUMAN | RLDLPPGFMFKVQAQHDYTATDTDELQLKAGDVVLVIPFQNPEEQ |
| | | DEGWLMGVKESDWNQHKELEKCRGVFPENFTERVP |
| 423 | ETV7_HUMAN | GICKLPGRLRIQPALWSREDVLHWLRWAEQEYSLPCTAEHGFEMN |
| | | GRALCILTKDDFRHRAPSSGDVLYELLQYIKTQRR |
| 424 | ASCL3_HUMAN | PNYRGCEYSYGPAFTRKRNERERQRVKCVNEGYAQLRHHLPEEYL |
| | | EKRLSKVETLRAAIKYINYLQSLLYPDKAETKNNP |
| 425 | PHC1_HUMAN | LHGINPVFLSSNPSRWSVEEVYEFIASLQGCQEIAEEFRSQEIDG |
| | | QALLLLKEEHLMSAMNIKLGPALKICAKINVLKET |
| 426 | OTP_HUMAN | QAGQQQGQQKQKRHRTRFTPAQLNELERSFAKTHYPDIFMREELA |
| | | LRIGLTESRVQVWFQNRRAKWKKRKKTTNVFRAPG |
| 427 | I2BP2_HUMAN | AAAVAVAAASRRQSCYLCDLPRMPWAMIWDFTEPVCRGCVNYEGA |
| | | DRVEFVIETARQLKRAHGCFPEGRSPPGAAASAAA |
| 428 | VGLL2_HUMAN | FSSQTPASIKEEEGSPEKERPPEAEYINSRCVLFTYFQGDISSVV |
| | | DEHFSRALSQPSSYSPSCTSSKAPRSSGPWRDCSF |
| 429 | HXA11_HUMAN | DKAGGSSGQRTRKKRCPYTKYQIRELEREFFFSVYINKEKRLQLS |
| | | RMLNLTDRQVKIWFQNRRMKEKKINRDRLQYYSAN |
| 430 | PDLI4_HUMAN | GAPLSGLQGLPECTRCGHGIVGTIVKARDKLYHPECFMCSDCGLN |
| | | LKQRGYFFLDERLYCESHAKARVKPPEGYDVVAVY |
| 431 | ASCL2_HUMAN | RRPATAETGGGAAAVARRNERERNRVKLVNLGFQALRQHVPHGGA |
| | | SKKLSKVETLRSAVEYIRALQRLLAEHDAVRNALA |
| 432 | CDX4_HUMAN | TVQVTGKTRTKEKYRVVYTDHQRLELEKEFHCNRYITIQRKSELA |
| | | VNLGLSERQVKIWFQNRRAKERKMIKKKISQFENS |
| 433 | ZN860_HUMAN | EEAAQKRKEKEPGMALPQGHLTFRDVAIEFSLEEWKCLDPTQRAL |
| | | YRAMMLENYRNLHSVDISSKCMMKKESSTAQGNTE |
| 434 | LMBL4_HUMAN | DIRASQVARWTVDEVAEFVQSLLGCEEHAKCFKKEQIDGKAFLLL |
| | | TQTDIVKVMKIKLGPALKIYNSILMFRHSQELPEE |
| 435 | PDIP3_HUMAN | LSPLEGTKMTVNNLHPRVTEEDIVELFCVCGALKRARLVHPGVAE |
| | | VVFVKKDDAITAYKKYNNRCLDGQPMKCNLHMNGN |
| 436 | NKX25_HUMAN | DNAERPRARRRRKPRVLFSQAQVYELERRFKQQRYLSAPERDQLA |
| | | SVLKLTSTQVKIWFQNRRYKCKRQRQDQTLELVGL |
| 437 | CEBPB_HUMAN | SQVKSKAKKTVDKHSDEYKIRRERNNIAVRKSRDKAKMRNLETQH |
| | | KVLELTAENERLQKKVEQLSRELSTLRNLFKQLPE |
| 438 | ISL1_HUMAN | KRDYIRLYGIKCAKCSIGFSKNDFVMRARSKVYHIECFRCVACSR |
| | | QLIPGDEFALREDGLFCRADHDVVERASLGAGDPL |
| 439 | CDX2_HUMAN | SLGSQVKTRTKDKYRVVYTDHQRLELEKEFHYSRYITIRRKAELA |
| | | ATLGLSERQVKIWFQNRRAKERKINKKKLQQQQQQ |
| 440 | PROP1_HUMAN | QGGQRGRPHSRRRHRTTFSPVQLEQLESAFGRNQYPDIWARESLA |
| | | RDTGLSEARIQVWFQNRRAKQRKQERSLLQPLAHL |
| 441 | SIN3B_HUMAN | DALTYLDQVKIRFGSDPATYNGFLEIMKEFKSQSIDTPGVIRRVS |
| | | QLFHEHPDLIVGFNAFLPLGYRIDIPKNGKLNIQS |
| 442 | SMBT1_HUMAN | RLHLDSNPLKWSVADVVRFIRSTDCAPLARIFLDQEIDGQALLLL |
| | | TLPTVQECMDLKLGPAIKLCHHIERIKFAFYEQFA |
| 443 | HXC11_HUMAN | AKGAAPNAPRTRKKRCPYSKFQIRELEREFFENVYINKEKRLQLS |
| | | RMLNLTDRQVKIWFQNRRMKEKKLSRDRLQYFSGN |
| 444 | HXC10_HUMAN | TTGNWLTAKSGRKKRCPYTKHQTLELEKEFLFNMYLTRERRLEIS |
| | | KTINLTDRQVKIWFQNRRMKLKKMNRENRIRELTS |
| 445 | PRS6A_HUMAN | YLVSNVIELLDVDPNDQEEDGANIDLDSQRKGKCAVIKTSTRQTY |
| | | FLPVIGLVDAEKLKPGDLVGVNKDSYLILETLPTE |
| 446 | VSX1_HUMAN | KASPTLGKRKKRRHRTVFTAHQLEELEKAFSEAHYPDVYAREMLA |
| | | VKTELPEDRIQVWFQNRRAKWRKREKRWGGSSVMA |
| 447 | NKX23_HUMAN | EESERPKPRSRRKPRVLFSQAQVFELERRFKQQRYLSAPEREHLA |
| | | SSLKLISTQVKIWFQNRRYKCKRQRQDKSLELGAH |
| 448 | MTG16_HUMAN | VVPGSRQEEVIDHKLTEREWAEEWKHLNNLLNCIMDMVEKTRRSL |
| | | TVLRRCQEADREELNHWARRYSDAEDTKKGPAPAA |
| 449 | HMX3_HUMAN | ESPEKKPACRKKKTRTVFSRSQVFQLESTFDMKRYLSSSERAGLA |
| | | ASLHLTETQVKIWFQNRRNKWKRQLAAELEAANLS |
| 450 | HMX1_HUMAN | RGGVGVGGGRKKKTRTVFSRSQVFQLESTEDLKRYLSSAERAGLA |
| | | ASLQLTETQVKIWFQNRRNKWKRQLAAELEAASLS |
| 451 | KIF22_HUMAN | ELLAHGRQKILDLLNEGSARDLRSLQRIGPKKAQLIVGWRELHGP |
| | | FSQVEDLERVEGITGKQMESFLKANILGLAAGQRC |
| 452 | CSTF2_HUMAN | ESPYGETISPEDAPESISKAVASLPPEQMFELMKQMKLCVQNSPQ |
| | | EARNMLLQNPQLAYALLQAQVVMRIVDPEIALKIL |
| 453 | CEBPE_HUMAN | AGPLHKGKKAVNKDSLEYRLRRERNNIAVRKSRDKAKRRILETQQ |
| | | KVLEYMAENERLRSRVEQLTQELDTLRNLFRQIPE |
| 454 | DLX2_HUMAN | IRIVNGKPKKVRKPRTIYSSFQLAALQRRFQKTQYLALPERAELA |
| | | ASLGLTQTQVKIWFQNRRSKFKKMWKSGEIPSEQH |
| 455 | ZMYM3_HUMAN | TVYQFCSPSCWTKFQRTSPEGGIHLSCHYCHSLFSGKPEVLDWQD |
| | | QVFQFCCRDCCEDFKRLRGVVSQCEHCRQEKLLHE |
| 456 | PPARG_HUMAN | TMVDTEMPFWPTNFGISSVDLSVMEDHSHSFDIKPFTTVDFSSIS |
| | | TPHYEDIPFTRTDPVVADYKYDLKLQEYQSAIKVE |
| 457 | PRIC1_HUMAN | GRHHAELLKPRCSACDEIIFADECTEAEGRHWHMKHFCCLECETV |
| | | LGGQRYIMKDGRPFCCGCFESLYAEYCETCGEHIG |
| 458 | UNC4_HUMAN | DPDKESPGCKRRRTRTNFTGWQLEELEKAFNESHYPDVFMREALA |
| | | LRLDLVESRVQVWFQNRRAKWRKKENTKKGPGRPA |
| 459 | BARX2_HUMAN | TEQPTPRQKKPRRSRTIFTELQLMGLEKKFQKQKYLSTPDRLDLA |
| | | QSLGLTQLQVKTWYQNRRMKWKKMVLKGGQEAPTK |
| 460 | ALX3_HUMAN | SMELAKNKSKKRRNRTTFSTFQLEELEKVFQKTHYPDVYAREQLA |
| | | LRTDLTEARVQVWFQNRRAKWRKRERYGKIQEGRN |
| 461 | TCF15_HUMAN | GGGGGAGPVVVVRQRQAANARERDRTQSVNTAFTALRTLIPTEPV |
| | | DRKLSKIETVRLASSYIAHLANVLLLGDSADDGQP |
| 462 | TERA_HUMAN | IDDTVEGITGNLFEVYLKPYFLEAYRPIRKGDIFLVRGGMRAVEF |
| | | KVVETDPSPYCIVAPDTVIHCEGEPIKREDEEESL |
| 463 | VSX2_HUMAN | SALNQTKKRKKRRHRTIFTSYQLEELEKAFNEAHYPDVYAREMLA |
| | | MKTELPEDRIQVWFQNRRAKWRKREKCWGRSSVMA |
| 464 | HXD12_HUMAN | DGLPWGAAPGRARKKRKPYTKQQIAELENEFLVNEFINRQKRKEL |
| | | SNRLNLSDQQVKIWFQNRRMKKKRVVLREQALALY |
| 465 | CDX1_HUMAN | GGGGSGKTRIKDKYRVVYTDHQRLELEKEFHYSRYITIRRKSELA |
| | | ANLGLTERQVKIWFQNRRAKERKVNKKKQQQQQPP |
| 466 | TCF23_HUMAN | TRAGGLALGRSEASPENAARERSRVRILRQAFLALQAALPAVPPD |
| | | TKLSKLDVLVLAASYIAHLTRILGHELPGPAWPPF |
| 467 | ALX1_HUMAN | KCDSNVSSSKKRRHRTTFTSLQLEELEKVFQKTHYPDVYVREQLA |
| | | LRTELTEARVQVWFQNRRAKWRKRERYGQIQQAKS |
| 468 | HXA10_HUMAN | NAANWLTAKSGRKKRCPYTKHQTLELEKEFLFNMYLTRERRLEIS |
| | | RSVHLTDRQVKIWFQNRRMKLKKMNRENRIRELTA |
| 469 | RX_HUMAN | LSEEEQPKKKHRRNRTTFTTYQLHELERAFEKSHYPDVYSREELA |
| | | GKVNLPEVRVQVWFQNRRAKWRRQEKLEVSSMKLQ |
| 470 | CXXC5_HUMAN | HMAGLAEYPMQGELASAISSGKKKRKRCGMCAPCRRRINCEQCSS |
| | | CRNRKTGHQICKFRKCEELKKKPSAALEKVMLPTG |
| 471 | SCML1_HUMAN | SITKHPSTWSVEAVVLFLKQTDPLALCPLVDLFRSHEIDGKALLL |
| | | LTSDVLLKHLGVKLGTAVKLCYYIDRLKQGKCFEN |
| 472 | NFIL3_HUMAN | ACRRKREFIPDEKKDAMYWEKRRKNNEAAKRSREKRRLNDLVLEN |
| | | KLIALGEENATLKAELLSLKLKFGLISSTAYAQEI |
| 473 | DLX6_HUMAN | EIRFNGKGKKIRKPRTIYSSLQLQALNHRFQQTQYLALPERAELA |
| | | ASLGLTQTQVKIWFQNKRSKFKKLLKQGSNPHESD |
| 474 | MTG8_HUMAN | GLHGTRQEEMIDHRLTDREWAEEWKHLDHLLNCIMDMVEKTRRSL |
| | | TVLRRCQEADREELNYWIRRYSDAEDLKKGGGSSS |
| 475 | CBX8_HUMAN | ELSAVGERVFAAEALLKRRIRKGRMEYLVKWKGWSQKYSTWEPEE |
| | | NILDARLLAAFEEREREMELYGPKKRGPKPKTFLL |
| 476 | CEBPD_HUMAN | AREKSAGKRGPDRGSPEYRQRRERNNIAVRKSRDKAKRRNQEMQQ |
| | | KLVELSAENEKLHQRVEQLTRDLAGLRQFFKQLPS |
| 477 | SEC13_HUMAN | SGGCDNLIKLWKEEEDGQWKEEQKLEAHSDWVRDVAWAPSIGLPT |
| | | STIASCSQDGRVFIWTCDDASSNTWSPKLLHKEND |
| 478 | FIP1_HUMAN | VKGVDLDAPGSINGVPLLEVDLDSFEDKPWRKPGADLSDYFNYGF |
| | | NEDTWKAYCEKQKRIRMGLEVIPVTSTINKITAED |
| 479 | ALX4_HUMAN | KADSESNKGKKRRNRTTFTSYQLEELEKVFQKTHYPDVYAREQLA |
| | | MRTDLTEARVQVWFQNRRAKWRKRERFGQMQQVRT |
| 480 | LHX3_HUMAN | TAKQREAEATAKRPRTTITAKQLETLKSAYNTSPKPARHVREQLS |
| | | SETGLDMRVVQVWFQNRRAKEKRLKKDAGRQRWGQ |
| 481 | PRIC2_HUMAN | GRHHAECLKPRCAACDEIIFADECTEAEGRHWHMKHFCCFECETV |
| | | LGGQRYIMKEGRPYCCHCFESLYAEYCDTCAQHIG |
| 482 | MAGI3_HUMAN | IIGGDRPDEFLQVKNVLKDGPAAQDGKIAPGDVIVDINGNCVLGH |
| | | THADVVQMFQLVPVNQYVNLTLCRGYPLPDDSEDP |
| 483 | NELL1_HUMAN | CCPECDTRVTSQCLDQNGHKLYRSGDNWTHSCQQCRCLEGEVDCW |
| | | PLICPNLSCEYTAILEGECCPRCVSDPCLADNITY |
| 484 | PRRX1_HUMAN | LNSEEKKKRKQRRNRTTENSSQLQALERVFERTHYPDAFVREDLA |
| | | RRVNLTEARVQVWFQNRRAKFRRNERAMLANKNAS |
| 485 | MTG8R_HUMAN | GLNGGYQDELVDHRLTEREWADEWKHLDHALNCIMEMVEKTRRSM |
| | | AVLRRCQESDREELNYWKRRYNENTELRKTGTELV |
| 486 | RAX2_HUMAN | GPGEEAPKKKHRRNRTTFTTYQLHQLERAFEASHYPDVYSREELA |
| | | AKVHLPEVRVQVWFQNRRAKWRRQERLESGSGAVA |
| 487 | DLX3_HUMAN | VRMVNGKPKKVRKPRTIYSSYQLAALQRRFQKAQYLALPERAELA |
| | | AQLGLTQTQVKIWFQNRRSKFKKLYKNGEVPLEHS |
| 488 | DLX1_HUMAN | EVRFNGKGKKIRKPRTIYSSLQLQALNRRFQQTQYLALPERAELA |
| | | ASLGLTQTQVKIWFQNKRSKFKKLMKQGGAALEGS |
| 489 | NKX26_HUMAN | GRSEQPKARQRRKPRVLFSQAQVLALERRFKQQRYLSAPEREHLA |
| | | SALQLISTQVKIWFQNRRYKCKRQRQDKSLELAGH |
| 490 | NAB1_HUMAN | LPRILGELQLYRILQKANLLSYFDAFIQQGGDDVQQLCEAGEEEF |
| | | LEIMALVGMASKPLHVRRLQKALRDWVINPGLENQ |
| 491 | SAMD7_HUMAN | NLSLDEDIQKWTVDDVHSFIRSLPGCSDYAQVFKDHAIDGETLPL |
| | | LTEEHLRGTMGLKLGPALKIQSQVSQHVGSMFYKK |
| 492 | PITX3_HUMAN | SPEDGSLKKKQRRQRTHFTSQQLQELEATFQRNRYPDMSTREEIA |
| | | VWTNLTEARVRVWFKNRRAKWRKRERSQQAELCKG |
| 493 | WDR5_HUMAN | SNLLVSASDDKTLKIWDVSSGKCLKTLKGHSNYVFCCNFNPQSNL |
| | | IVSGSFDESVRIWDVKTGKCLKTLPAHSDPVSAVH |
| 494 | MEOX2_HUMAN | GNYKSEVNSKPRKERTAFTKEQIRELEAEFAHHNYLTRLRRYEIA |
| | | VNLDLTERQVKVWFQNRRMKWKRVKGGQQGAAARE |
| 495 | NAB2_HUMAN | LPRTLGELQLYRVLQRANLLSYYETFIQQGGDDVQQLCEAGEEEF |
| | | LEIMALVGMATKPLHVRRLQKALREWATNPGLFSQ |
| 496 | DHX8_HUMAN | PEEPTIGDIYNGKVTSIMQFGCFVQLEGLRKRWEGLVHISELRRE |
| | | GRVANVADVVSKGQRVKVKVLSFTGTKTSLSMKDV |
| 497 | FOXA2_HUMAN | YAFNHPFSINNLMSSEQQHHHSHHHHQPHKMDLKAYEQVMHYPGY |
| | | GSPMPGSLAMGPVINKTGLDASPLAADTSYYQGVY |
| 498 | CBX6_HUMAN | TAAAGPAPPTAPEPAGASSEPEAGDWRPEMSPCSNVVVTDVTSNL |
| | | LTVTIKEFCNPEDFEKVAAGVAGAAGGGGSIGASK |
| 499 | EMX2_HUMAN | FLLHNALARKPKRIRTAFSPSQLLRLEHAFEKNHYVVGAERKQLA |
| | | HSLSLTETQVKVWFQNRRTKFKRQKLEEEGSDSQQ |
| 500 | CPSF6_HUMAN | KRIALYIGNLTWWTTDEDLTEAVHSLGVNDILEIKFFENRANGQS |
| | | KGFALVGVGSEASSKKLMDLLPKRELHGQNPVVTP |
| 501 | HXC12_HUMAN | SGAPWYPINSRSRKKRKPYSKLQLAELEGEFLVNEFITRQRRREL |
| | | SDRLNLSDQQVKIWFQNRRMKKKRLLLREQALSFF |
| 502 | KDM4B_HUMAN | SDNLYPESITSRDCVQLGPPSEGELVELRWTDGNLYKAKFISSVT |
| | | SHIYQVEFEDGSQLTVKRGDIFTLEEELPKRVRSR |
| 503 | LMBL3_HUMAN | GIPASKVSKWSTDEVSEFIQSLPGCEEHGKVFKDEQIDGEAFLLM |
| | | TQTDIVKIMSIKLGPALKIFNSILMFKAAEKNSHN |
| 504 | PHX2A_HUMAN | EPSGLHEKRKQRRIRTTFTSAQLKELERVFAETHYPDIYTREELA |
| | | LKIDLTEARVQVWFQNRRAKFRKQERAASAKGAAG |
| 505 | EMX1_HUMAN | LLLHGPFARKPKRIRTAFSPSQLLRLERAFEKNHYVVGAERKQLA |
| | | GSLSLSETQVKVWFQNRRTKYKRQKLEEEGPESEQ |
| 506 | NC2B_HUMAN | SSGNDDDLTIPRAAINKMIKETLPNVRVANDARELVVNCCTEFIH |
| | | LISSEANEICNKSEKKTISPEHVIQALESLGFGSY |
| 507 | DLX4_HUMAN | ERRPQAPAKKLRKPRTIYSSLQLQHLNQRFQHTQYLALPERAQLA |
| | | AQLGLTQTQVKIWFQNKRSKYKKLLKQNSGGQEGD |
| 508 | SRY_HUMAN | NVQDRVKRPMNAFIVWSRDQRRKMALENPRMRNSEISKQLGYQWK |
| | | MLTEAEKWPFFQEAQKLQAMHREKYPNYKYRPRRK |
| 509 | ZN777_HUMAN | EITRLAVWAAVQAVERKLEAQAMRLLTLEGRIGTNEKKIADCEKT |
| | | AVEFANHLESKWVVLGILLQEYGLLQRRLENMENL |
| 510 | NELL1_HUMAN | CEKDIDECSEGIIECHNHSRCVNLPGWYHCECRSGFHDDGTYSLS |
| | | GESCIDIDECALRTHTCWNDSACINLAGGFDCLCP |
| 511 | ZN398_HUMAN | AAISLWTVVAAVQAIERKVEIHSRRLLHLEGRTGTAEKKLASCEK |
| | | TVTELGNQLEGKWAVLGTILLQEYGLLQRRLENLEN |
| 512 | GATA3_HUMAN | GQNRPLIKPKRRLSAARRAGTSCANCQTTTTTLWRRNANGDPVCN |
| | | ACGLYYKLHNINRPLIMKKEGIQTRNRKMSSKSKK |
| 513 | BSH_HUMAN | HAELPGKHCRRRKARTVFSDSQLSGLEKRFEIQRYLSTPERVELA |
| | | TALSLSETQVKTWFQNRRMKHKKQLRKSQDEPKAP |
| 514 | SF3B4_HUMAN | QDATVYVGGLDEKVSEPLLWELFLQAGPVVNTHMPKDRVTGQHQG |
| | | YGFVEFLSEEDADYAIKIMNMIKLYGKPIRVNKAS |
| 515 | TEAD1_HUMAN | PIDNDAEGVWSPDIEQSFQEALAIYPPCGRRKIILSDEGKMYGRN |
| | | ELIARYIKLRTGKTRTRKQVSSHIQVLARRKSRDF |
| 516 | TEAD3_HUMAN | GLDNDAEGVWSPDIEQSFQEALAIYPPCGRRKIILSDEGKMYGRN |
| | | ELIARYIKLRTGKTRTRKQVSSHIQVLARKKVREY |
| 517 | RGAP1_HUMAN | DSVGTPQSNGGMRLHDFVSKIVIKPESCVPCGKRIKFGKLSLKCR |
| | | DCRVVSHPECRDRCPLPCIPTLIGTPVKIGEGMLA |
| 518 | PHF1_HUMAN | SAPHSMTASSSSVSSPSPGLPRRSAPPSPLCRSLSPGTGGGVRGG |
| | | VGYLSRGDPVRVLARRVRPDGSVQYLVEWGGGGIF |
| 519 | FOXA1_HUMAN | GDPHYSFNHPFSINNLMSSSEQQHKLDFKAYEQALQYSPYGSTLP |
| | | ASLPLGSASVTTRSPIEPSALEPAYYQGVYSRPVL |
| 520 | GATA2_HUMAN | GQNRPLIKPKRRLSAARRAGTCCANCQTTTTTLWRRNANGDPVCN |
| | | ACGLYYKLHNVNRPLIMKKEGIQTRNRKMSNKSKK |
| 521 | FOXO3_HUMAN | DSLSGSSLYSTSANLPVMGHEKFPSDLDLDMENGSLECDMESIIR |
| | | SELMDADGLDFNFDSLISTQNVVGLNVGNFTGAKQ |
| 522 | ZN212_HUMAN | TEISLWTVVAAIQAVEKKMESQAARLQSLEGRTGTAEKKLADCEK |
| | | MAVEFGNQLEGKWAVLGTLLQEYGLLQRRLENVEN |
| 523 | IRX4_HUMAN | MDSGTRRKNATRETTSTLKAWLQEHRKNPYPTKGEKIMLAIITKM |
| | | TLTQVSTWFANARRRLKKENKMTWPPRNKCADEKR |
| 524 | ZBED6_HUMAN | NIEKQIYLPSTRAKTSIVWHFFHVDPQYTWRAICNLCEKSVSRGK |
| | | PGSHLGTSTLQRHLQARHSPHWTRANKFGVASGEE |
| 525 | LHX4_HUMAN | AKQNDDSEAGAKRPRITITAKQLETLKNAYKNSPKPARHVREQLS |
| | | SETGLDMRVVQVWFQNRRAKEKRLKKDAGRHRWGQ |
| 526 | SIN3A_HUMAN | DALSYLDQVKLQFGSQPQVYNDFLDIMKEFKSQSIDTPGVISRVS |
| | | QLFKGHPDLIMGFNTFLPPGYKIEVQTNDMVNVTT |
| 527 | RBBP7_HUMAN | DDHTVCLWDINAGPKEGKIVDAKAIFTGHSAVVEDVAWHLLHESL |
| | | FGSVADDQKLMIWDTRSNTTSKPSHLVDAHTAEVN |
| 528 | NKX61_HUMAN | GSILLDKDGKRKHTRPTFSGQQIFALEKTFEQTKYLAGPERARLA |
| | | YSLGMTESQVKVWFQNRRTKWRKKHAAEMATAKKK |
| 529 | TRI68_HUMAN | DPTALVEAIVEEVACPICMTFLREPMSIDCGHSFCHSCLSGLWEI |
| | | PGESQNWGYTCPLCRAPVQPRNLRPNWQLANVVEK |
| 530 | R51A1_HUMAN | QSLPKKVSLSSDTTRKPLEIRSPSAESKKPKWVPPAASGGSRSSS |
| | | SPLVVVSVKSPNQSLRLGLSRLARVKPLHPNATST |
| 531 | MB3L1_HUMAN | AKSSQRKQRDCVNQCKSKPGLSTSIPLRMSSYTFKRPVTRITPHP |
| | | GNEVRYHQWEESLEKPQQVCWQRRLQGLQAYSSAG |
| 532 | DLX5_HUMAN | VRMVNGKPKKVRKPRTIYSSFQLAALQRRFQKTQYLALPERAELA |
| | | ASLGLTQTQVKIWFQNKRSKIKKIMKNGEMPPEHS |
| 533 | NOTC1_HUMAN | LQCNNHACGWDGGDCSLNFNDPWKNCTQSLQCWKYFSDGHCDSQC |
| | | NSAGCLFDGFDCQRAEGQCNPLYDQYCKDHFSDGH |
| 534 | TERF2_HUMAN | ETWVEEDELFQVQAAPDEDSTTNITKKQKWTVEESEWVKAGVQKY |
| | | GEGNWAAISKNYPFVNRTAVMIKDRWRIMKRLGMN |
| 535 | ZN282_HUMAN | AEISLWTVVAAIQAVERKVDAQASQLLNLEGRTGTAEKKLADCEK |
| | | TAVEFGNHMESKWAVLGTLLQEYGLLQRRLENLEN |
| 536 | RGS12_HUMAN | LEKRTLFRLDLVPINRSVGLKAKPTKPVTEVLRPVVARYGLDLSG |
| | | LLVRLSGEKEPLDLGAPISSLDGQRVVLEEKDPSR |
| 537 | ZN840_HUMAN | PNCLSSSMQLPHGGGRHQELVRFRDVAVVFSPEEWDHLTPEQRNL |
| | | YKDVMLDNCKYLASLGNWTYKAHVMSSLKQGKEPW |
| 538 | SPI2B_HUMAN | DDYKEGDLRIMPESSESPPTEREPGGVVDGLIGKHVEYTKEDGSK |
| | | RIGMVIHQVEAKPSVYFIKFDDDFHIYVYDLVKKS |
| 539 | PAX7_HUMAN | SEPDLPLKRKQRRSRITFTAEQLEELEKAFERTHYPDIYTREELA |
| | | QRTKLTEARVQVWFSNRRARWRKQAGANQLAAFNH |
| 540 | NKX62_HUMAN | AGGVLDKDGKKKHSRPTFSGQQIFALEKTFEQTKYLAGPERARLA |
| | | YSLGMTESQVKVWFQNRRTKWRKRHAVEMASAKKK |
| 541 | ASXL2_HUMAN | DVMSFSVTVTTIPASQAMNPSSHGQTIPVQAFSEENSIEGTPSKC |
| | | YCRLKAMIMCKGCGAFCHDDCIGPSKLCVSCLVVR |
| 542 | FOXO1_HUMAN | GGYSSVSSCNGYGRMGLLHQEKLPSDLDGMFIERLDCDMESIIRN |
| | | DLMDGDILDFNFDNVLPNQSFPHSVKTTTHSWVSG |
| 543 | GATA3_HUMAN | GGSPTGFGCKSRPKARSSTGRECVNCGATSTPLWRRDGTGHYLCN |
| | | ACGLYHKMNGQNRPLIKPKRRLSAARRAGTSCANC |
| 544 | GATA1_HUMAN | GQNRPLIRPKKRLIVSKRAGTQCTNCQTTTTTLWRRNASGDPVCN |
| | | ACGLYYKLHQVNRPLTMRKDGIQTRNRKASGKGKK |
| 545 | ZMYM5_HUMAN | PVALLRKQNFQPTAQQQLTKPAKITCANCKKPLQKGQTAYQRKGS |
| | | AHLFCSTTCLSSFSHKRTQNTRSIICKKDASTKKA |
| 546 | ZN783_HUMAN | TEITLWTVVAAIQALEKKVDSCLTRLLTLEGRTGTAEKKLADCEK |
| | | TAVEFGNQLEGKWAVLGILLQEYGLLQRRLENVEN |
| 547 | SPI2B_HUMAN | KKQRGRPSSQPRRNIVGCRISHGWKEGDEPITQWKGTVLDQVPIN |
| | | PSLYLVKYDGIDCVYGLELHRDERVLSLKILSDRV |
| 548 | LRP1_HUMAN | WTCDLDDDCGDRSDESASCAYPTCFPLTQFTCNNGRCININWRCD |
| | | NDNDCGDNSDEAGCSHSCSSTQFKCNSGRCIPEHW |
| 549 | MIXL1_HUMAN | PKGAAAPSASQRRKRISFSAEQLQLLELVFRRTRYPDIHLRERLA |
| | | ALTLLPESRIQVWFQNRRAKSRRQSGKSFQPLARP |
| 550 | SGT1_HUMAN | KIKYDWYQTESQVVITLMIKNVQKNDVNVEFSEKELSALVKLPSG |
| | | EDYNLKLELLHPIIPEQSTFKVLSTKIEIKLKKPE |
| 551 | LMCD1_HUMAN | DPSKEVEYVCELCKGAAPPDSPVVYSDRAGYNKQWHPTCFVCAKC |
| | | SEPLVDLIYFWKDGAPWCGRHYCESLRPRCSGCDE |
| 552 | CEBPA_HUMAN | GSGAGKAKKSVDKNSNEYRVRRERNNIAVRKSRDKAKQRNVETQQ |
| | | KVLELTSDNDRLRKRVEQLSRELDTLRGIFRQLPE |
| 553 | GATA2_HUMAN | GPASSFTPKQRSKARSCSEGRECVNCGATATPLWRRDGTGHYLCN |
| | | ACGLYHKMNGQNRPLIKPKRRLSAARRAGTCCANC |
| 554 | SOX14_HUMAN | KPSDHIKRPMNAFMVWSRGQRRKMAQENPKMHNSEISKRLGAEWK |
| | | LLSEAEKRPYIDEAKRLRAQHMKEHPDYKYRPRRK |
| 555 | WTIP_HUMAN | LYSGFQQTADKCSVCGHLIMEMILQALGKSYHPGCFRCSVCNECL |
| | | DGVPFTVDVENNIYCVRDYHTVFAPKCASCARPIL |
| 556 | PRP19_HUMAN | HPSQDLVFSASPDATIRIWSVPNASCVQVVRAHESAVTGLSLHAT |
| | | GDYLLSSSDDQYWAFSDIQTGRVLTKVTDETSGCS |
| 557 | CBX6_HUMAN | ELSAVGERVFAAESIIKRRIRKGRIEYLVKWKGWAIKYSTWEPEE |
| | | NILDSRLIAAFEQKERERELYGPKKRGPKPKTFLL |
| 558 | NKX11_HUMAN | RTGSDSKSGKPRRARTAFTYEQLVALENKFKATRYLSVCERLNLA |
| | | LSLSLTETQVKIWFQNRRTKWKKQNPGADTSAPTG |
| 559 | RBBP4_HUMAN | VWDLSKIGEEQSPEDAEDGPPELLFIHGGHTAKISDFSWNPNEPW |
| | | VICSVSEDNIMQVWQMAENIYNDEDPEGSVDPEGQ |
| 560 | DMRT2_HUMAN | ERCTPAGGGAEPRKLSRTPKCARCRNHGVVSCLKGHKRFCRWRDC |
| | | QCANCLLVVERQRVMAAQVALRRQQATEDKKGLSG |
| 561 | SMCA2_HUMAN | SQPGALIPGDPQAMSQPNRGPSPFSPVQLHQLRAQILAYKMLARG |
| | | QPLPETLQLAVQGKRILPGLQQQQQ |
| 562 | ZNF10 | MDAKSLTAWSRTLVTFKDVFVDFTREEWKLLDTAQQIVYRNVMLE |
| | | NYKNLVSLGYQLTKPDVILRLEKGEEPWLVEREIHQETHPDSETA |
| | | FEIKSSVSSRSIFKDKQSCDIKMEGMARNDLWYLSLEEVWKCRDQ |
| | | LDKYQENPERHLRQVAFTQKKVLTQERVSESGKYGGNCLLPAQLV |
| | | LREYFHKRDSHTKSLKHDLVLNGHQDSCASNSNECGQTFCQNIHL |
| | | IQFARTHTGDKSYKCPDNDNSLTHGSSLGISKGIHREKPYECKEC |
| | | GKFFSWRSNLTRHQLIHTGEKPYECKECGKSFSRSSHLIGHQKTH |
| | | TGEEPYECKECGKSFSWFSHLVTHQRTHTGDKLYTCNQCGKSFVH |
| | | SSRLIRHQRTHTGEKPYECPECGKSFRQSTHLILHQRTHVRVRPY |
| | | ECNECGKSYSQRSHLVVHHRIHTGLKPFECKDCGKCFSRSSHLYS |
| | | HQRTHTGEKPYECHDCGKSFSQSSALIVHQRIHTGEKPYECCQCG |
| | | KAFIRKNDLIKHQRIHVGEETYKCNQCGIIFSQNSPFIVHQIAHT |
| | | GEQFLTCNQCGTALVNTSNLIGYQTNHIRENAY |
| 563 | EED_HUMAN | MSEREVSTAPAGTDMPAAKKQKLSSDENSNPDLSGDENDDAVSIE |
| | | SGTNTERPDTPTNTPNAPGRKSWGKGKWKSKKCKYSFKCVNSLKE |
| | | DHNQPLFGVQFNWHSKEGDPLVFATVGSNRVTLYECHSQGEIRLL |
| | | QSYVDADADENFYTCAWTYDSNTSHPLLAVAGSRGIIRIINPITM |
| | | QCIKHYVGHGNAINELKFHPRDPNLLLSVSKDHALRLWNIQTDTL |
| | | VAIFGGVEGHRDEVLSADYDLLGEKIMSCGMDHSLKLWRINSKRM |
| | | MNAIKESYDYNPNKINRPFISQKIHFPDFSTRDIHRNYVDCVRWL |
| | | GDLILSKSCENAIVCWKPGKMEDDIDKIKPSESNVTILGRFDYSQ |
| | | CDIWYMRFSMDFWQKMLALGNQVGKLYVWDLEVEDPHKAKCTTLT |
| | | HHKCGAAIRQTSFSRDSSILIAVCDDASIWRWDRLR |
| 564 | RCOR1_HUMAN | MPAMVEKGPEVSGKRRGRNNAAASASAAAASAAASAACASPAATA |
| | | ASGAAASSASAAAASAAAAPNNGQNKSLAAAAPNGNSSSNSWEEG |
| | | SSGSSSDEEHGGGGMRVGPQYQAVVPDFDPAKLARRSQERDNLGM |
| | | LVWSPNQNLSEAKLDEYIAIAKEKHGYNMEQALGMLFWHKHNIEK |
| | | SLADLPNFTPFPDEWTVEDKVLFEQAFSFHGKTFHRIQQMLPDKS |
| | | IASLVKFYYSWKKTRIKTSVMDRHARKQKREREESEDELEEANGN |
| | | NPIDIEVDQNKESKKEVPPTETVPQVKKEKHSTQAKNRAKRKPPK |
| | | GMFLSQEDVEAVSANATAATTVLRQLDMELVSVKRQIQNIKQTNS |
| | | ALKEKLDGGIEPYRLPEVIQKCNARWTTEEQLLAVQAIRKYGRDF |
| | | QAISDVIGNKSVVQVKNFFVNYRRRFNIDEVLQEWEAEHGKEETN |
| | | GPSNQKPVKSPDNSIKMPEEEDEAPVLDVRYASAS |
| 565 | KOX1/ZNF10 | TGRTLVTFKDVFVDFTREEWKLLDTAQQIVYRNVMLENYKNLVSL |
| | KRAB 1 | GYQLTKPDVILRLEKGEEPLEINLWITKFVKD |
| 566 | KOX1/ZNF10 | MYPYDVPDYASPKKKRKVGGGASMDAKSLTAWSRTLVTFKDVFVD |
| | KRAB 2 | FTREEWKLLDTAQQIVYRNVMLENYKNLVSLGYQLTKPDVILRLE |
| | | KGEEPWLVEREIHQETHPDSETAFEIKSSV |
| 567 | KOX1/ZNF10 | ALSPQHSAVTQGSIIKNKEGMDAKSLTAWSRTLVTFKDVFVDFTR |
| | KRAB 3 | EEWKLLDTAQQIVYRNVMLENYKNLVSLGYQLTKPDVILRLEKGE |
| | | EPWLVEREIHQETHPDSETAFEIKSSV |
| 568 | KOX1/ZNF10 (aa | RTLVTFKDVFVDFTREEWKLLDTAQQIVYRNVMLENYKNLVSLGY |
| | 11-72) | QLTKPDVILRLEKGEEP |
| 569 | KOX1/ZNF10 (aa | RTLVTFKDVFVDFTREEWKLLDTAQQIVYRNVMLENYKNLVSLGY |
| | 11-108) | QLTKPDVILRLEKGEEPWLVEREIHQETHPDSETAFEIKSSVSSR |
| | | SIFKDKQS |
| 570 | KOX1/ZNF10 | RTLVTFKDVAVDFTQEEWQQLDPAQKIVYRDVMLENYSNLVSVGY |
| | variant | QLTKPDVILRLEQKGEEPWLVEEEIHQETHPDSETAFEIKSSVSS |
| | | RSIFKDKQS |
| 571 | KOX1 KRAB-ZIM3 | RTLVTFKDVFVDFTREEWKLLDTAQQIVYRNVMLENYKNLVSLGY |
| | chimera | QLTKPDVILRLEKGEEPWLEEEEVLGSGRAEKNGDIGGQIWKPKD |
| | | VKESL |
| 572 | ZIM3-KOX1 KRAB | MNNSQGRVTFEDVTVNFTQGEWQRLNPEQRNLYRDVMLENYSNLV |
| | chimera | SVGQGETTKPDVILRLEQGKEPWLVEREIHQETHPDSETAFEIKS |
| | | SVSSRSIFKDKQS |
| 573 | human DNMT1 | MPARTAPARVPTLAVPAISLPDDVRRRLKDLERDSLTEKECVKEK |
| | | LNLLHEFLQTEIKNQLCDLETKLRKEELSEEGYLAKVKSLLNKDL |
| | | SLENGAHAYNREVNGRLENGNQARSEARRVGMADANSPPKPLSKP |
| | | RTPRRSKSDGEAKPEPSPSPRITRKSTRQTTITSHFAKGPAKRKP |
| | | QEESERAKSDESIKEEDKDQDEKRRRVISRERVARPLPAEEPERA |
| | | KSGTRTEKEEERDEKEEKRLRSQTKEPTPKQKLKEEPDREARAGV |
| | | QADEDEDGDEKDEKKHRSQPKDLAAKRRPEEKEPEKVNPQISDEK |
| | | DEDEKEEKRRKTTPKEPTEKKMARAKTVMNSKTHPPKCIQCGQYL |
| | | DDPLKYGQHPPDAVDEPQMLINEKLSIFDANESGFESYEALPQHK |
| | | LTCFSVYCKHGHLCPIDTGLIEKNIELFFSGSAKPIYDDDPSLEG |
| | | GVNGKNLGPINEWWITGFDGGEKALIGFSTSFAEYILMDPSPEYA |
| | | PIFGLMQEKIYISKIVVEFLQSNSDSTYEDLINKIETTVPPSGLN |
| | | LNRFTEDSLLRHAQFVVEQVESYDEAGDSDEQPIFLTPCMRDLIK |
| | | LAGVTLGQRRAQARRQTIRHSTREKDRGPTKATTTKLVYQIFDTF |
| | | FAEQIEKDDREDKENAFKRRRCGVCEVCQQPECGKCKACKDMVKF |
| | | GGSGRSKQACQERRCPNMAMKEADDDEEVDDNIPEMPSPKKMHQG |
| | | KKKKQNKNRISWVGEAVKTDGKKSYYKKVCIDAETLEVGDCVSVI |
| | | PDDSSKPLYLARVTALWEDSSNGQMFHAHWFCAGTDTVLGATSDP |
| | | LELFLVDECEDMQLSYIHSKVKVIYKAPSENWAMEGGMDPESLLE |
| | | GDDGKTYFYQLWYDQDYARFESPPKTQPTEDNKFKFCVSCARLAE |
| | | MRQKEIPRVLEQLEDLDSRVLYYSATKNGILYRVGDGVYLPPEAF |
| | | TFNIKLSSPVKRPRKEPVDEDLYPEHYRKYSDYIKGSNLDAPEPY |
| | | RIGRIKEIFCPKKSNGRPNETDIKIRVNKFYRPENTHKSTPASYH |
| | | ADINLLYWSDEEAVVDFKAVQGRCTVEYGEDLPECVQVYSMGGPN |
| | | RFYFLEAYNAKSKSFEDPPNHARSPGNKGKGKGKGKGKPKSQACE |
| | | PSEPEIEIKLPKLRTLDVFSGCGGLSEGFHQAGISDTLWAIEMWD |
| | | PAAQAFRLNNPGSTVFTEDCNILLKLVMAGETTNSRGQRLPQKGD |
| | | VEMLCGGPPCQGFSGMNRFNSRTYSKFKNSLVVSFLSYCDYYRPR |
| | | FFLLENVRNFVSFKRSMVLKLTLRCLVRMGYQCTFGVLQAGQYGV |
| | | AQTRRRAIILAAAPGEKLPLFPEPLHVFAPRACQLSVVVDDKKFV |
| | | SNITRLSSGPFRTITVRDTMSDLPEVRNGASALEISYNGEPQSWF |
| | | QRQLRGAQYQPILRDHICKDMSALVAARMRHIPLAPGSDWRDLPN |
| | | IEVRLSDGTMARKLRYTHHDRKNGRSSSGALRGVCSCVEAGKACD |
| | | PAARQFNTLIPWCLPHTGNRHNHWAGLYGRLEWDGFFSTTVINPE |
| | | PMGKQGRVLHPEQHRVVSVRECARSQGFPDTYRLFGNILDKHRQV |
| | | GNAVPPPLAKAIGLEIKLCMLAKARESASAKIKEEEAAKD |
| 574 | human DNMT3A | MPAMPSSGPGDTSSSAAEREEDRKDGEEQEEPRGKEERQEPSTTA |
| | | RKVGRPGRKRKHPPVESGDTPKDPAVISKSPSMAQDSGASELLPN |
| | | GDLEKRSEPQPEEGSPAGGQKGGAPAEGEGAAETLPEASRAVENG |
| | | CCTPKEGRGAPAEAGKEQKETNIESMKMEGSRGRLRGGLGWESSL |
| | | RQRPMPRLTFQAGDPYYISKRKRDEWLARWKREAEKKAKVIAGMN |
| | | AVEENQGPGESQKVEEASPPAVQQPTDPASPTVATTPEPVGSDAG |
| | | DKNATKAGDDEPEYEDGRGFGIGELVWGKLRGFSWWPGRIVSWWM |
| | | TGRSRAAEGTRWVMWFGDGKFSVVCVEKLMPLSSFCSAFHQATYN |
| | | KQPMYRKAIYEVLQVASSRAGKLFPVCHDSDESDTAKAVEVQNKP |
| | | MIEWALGGFQPSGPKGLEPPEEEKNPYKEVYTDMWVEPEAAAYAP |
| | | PPPAKKPRKSTAEKPKVKEIIDERTRERLVYEVRQKCRNIEDICI |
| | | SCGSLNVTLEHPLFVGGMCQNCKNCFLECAYQYDDDGYQSYCTIC |
| | | CGGREVLMCGNNNCCRCFCVECVDLLVGPGAAQAAIKEDPWNCYM |
| | | CGHKGTYGLLRRREDWPSRLQMFFANNHDQEFDPPKVYPPVPAEK |
| | | RKPIRVLSLFDGIATGLLVLKDLGIQVDRYIASEVCEDSITVGMV |
| | | RHQGKIMYVGDVRSVTQKHIQEWGPFDLVIGGSPCNDLSIVNPAR |
| | | KGLYEGTGRLFFEFYRLLHDARPKEGDDRPFFWLFENVVAMGVSD |
| | | KRDISRFLESNPVMIDAKEVSAAHRARYFWGNLPGMNRPLASTVN |
| | | DKLELQECLEHGRIAKFSKVRTITTRSNSIKQGKDQHFPVEMNEK |
| | | EDILWCTEMERVFGFPVHYTDVSNMSRLARQRLLGRSWSVPVIRH |
| | | LFAPLKEYFACV |
| 575 | human DNMT3A | NHDQEFDPPKVYPPVPAEKRKPIRVLSLFDGIATGLLVLKDLGIQ |
| | catalytic domain | VDRYIASEVCEDSITVGMVRHQGKIMYVGDVRSVTQKHIQEWGPF |
| | | DLVIGGSPCNDLSIVNPARKGLYEGTGRLFFEFYRLLHDARPKEG |
| | | DDRPFFWLFENVVAMGVSDKRDISRFLESNPVMIDAKEVSAAHRA |
| | | RYFWGNLPGMNRPLASTVNDKLELQECLEHGRIAKFSKVRTITTR |
| | | SNSIKQGKDQHFPVFMNEKEDILWCTEMERVFGFPVHYTDVSNMS |
| | | RLARQRLLGRSWSVPVIRHLFAPLKEYFACV |
| 576 | human DNMT3B | MKGDTRHLNGEEDAGGREDSILVNGACSDQSSDSPPILEAIRTPE |
| | | IRGRRSSSRLSKREVSSLLSYTQDLTGDGDGEDGDGSDTPVMPKL |
| | | FRETRTRSESPAVRTRNNNSVSSRERHRPSPRSTRGRQGRNHVDE |
| | | SPVEFPATRSLRRRATASAGTPWPSPPSSYLTIDLTDDTEDTHGT |
| | | PQSSSTPYARLAQDSQQGGMESPQVEADSGDGDSSEYQDGKEFGI |
| | | GDLVWGKIKGFSWWPAMVVSWKATSKRQAMSGMRWVQWFGDGKFS |
| | | EVSADKLVALGLFSQHFNLATFNKLVSYRKAMYHALEKARVRAGK |
| | | TFPSSPGDSLEDQLKPMLEWAHGGFKPTGIEGLKPNNTQPVVNKS |
| | | KVRRAGSRKLESRKYENKTRRRTADDSATSDYCPAPKRLKINCYN |
| | | NGKDRGDEDQSREQMASDVANNKSSLEDGCLSCGRKNPVSFHPLF |
| | | EGGLCQTCRDRFLELFYMYDDDGYQSYCTVCCEGRELLLCSNTSC |
| | | CRCFCVECLEVLVGTGTAAEAKLQEPWSCYMCLPQRCHGVLRRRK |
| | | DWNVRLQAFFTSDTGLEYEAPKLYPAIPAARRRPIRVLSLFDGIA |
| | | TGYLVLKELGIKVGKYVASEVCEESIAVGTVKHEGNIKYVNDVRN |
| | | ITKKNIEEWGPFDLVIGGSPCNDLSNVNPARKGLYEGTGRLFFEF |
| | | YHLLNYSRPKEGDDRPFFWMFENVVAMKVGDKRDISRFLECNPVM |
| | | IDAIKVSAAHRARYFWGNLPGMNRPVIASKNDKLELQDCLEYNRI |
| | | AKLKKVQTITTKSNSIKQGKNQLFPVVMNGKEDVLWCTELERIFG |
| | | FPVHYTDVSNMGRGARQKLLGRSWSVPVIRHLFAPLKDYFACE |
| 577 | mouse DNMT3C | MRGGSRHLSNEEDVSGCEDCIIISGTCSDQSSDPKTVPLTQVLEA |
| | | VCTVENRGCRISSQPSKRKASSLISYVQDLIGDGDEDRDGEVGGS |
| | | SGSGTPVMPQLFCETRIPSKTPAPLSWQANTSASTPWLSPASPYP |
| | | IIDLTDEDVIPQSISTPSVDWSQDSHQEGMDTTQVDAESRDGGNI |
| | | EYQVSADKLLLSQSCILAAFYKLVPYRESTYRTLEKARVRAGKAC |
| | | PSSPGESLEDQLKPMLEWAHGGFKPTGIEGLKPNKKQPENKSRRR |
| | | TTNDPAASESSPPKRLKINSYGGKDRGEDEESREQMASDVINNKG |
| | | NLEDHCLSCGRKDPVSFHPLFEGGLCQSCRDRFLELFYMYDEDGY |
| | | QSYCTVCCEGRELLLCSNTSCCRCFCVECLEVLVGAGTAEDVKLQ |
| | | EPWSCYMCLPQRCHGVLRRRKDWNMRLQDFFTTDPDLEEFEPPKL |
| | | YPAIPAAKRRPIRVLSLFDGIATGYLVLKELGIKVEKYIASEVCA |
| | | ESIAVGTVKHEGQIKYVDDIRNITKEHIDEWGPFDLVIGGSPCND |
| | | LSCVNPVRKGLFEGTGRLFFEFYRLLNYSCPEEEDDRPFFWMFEN |
| | | VVAMEVGDKRDISRFLECNPVMIDAIKVSAAHRARYFWGNLPGMN |
| | | RPVMASKNDKLELQDCLEFSRTAKLKKVQTITTKSNSIRQGKNQL |
| | | FPVVMNGKDDVLWCTELERIFGFPEHYTDVSNMGRGARQKLLGRS |
| | | WSVPVIRHLFAPLKDHFACE |
| 578 | human DNMT3L | MAAIPALDPEAEPSMDVILVGSSELSSSVSPGTGRDLIAYEVKAN |
| | | QRNIEDICICCGSLQVHTQHPLFEGGICAPCKDKFLDALFLYDDD |
| | | GYQSYCSICCSGETLLICGNPDCTRCYCFECVDSLVGPGTSGKVH |
| | | AMSNWVCYLCLPSSRSGLLQRRRKWRSQLKAFYDRESENPLEMFE |
| | | TVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYA |
| | | RPKPGSPRPFFWMFVDNLVLNKEDLDVASRFLEMEPVTIPDVHGG |
| | | SLQNAVRVWSNIPAIRSSRHWALVSEEELSLLAQNKQSSKLAAKW |
| | | PTKLVKNCFLPLREYFKYFSTELTSSL |
| 579 | human DNMT3L | NPLEMFETVPVWRRQPVRVLSLFEDIKKELTSLGFLESGSDPGQL |
| | catalytic domain | KHVVDVTDTVRKDVEEWGPFDLVYGATPPLGHTCDRPPSWYLFQF |
| | | HRLLQYARPKPGSPRPFFWMFVDNLVLNKEDLDVASRFLEMEPVT |
| | | IPDVHGGSLQNAVRVWSNIPAIRSRHWALVSEEELSLLAQNKQSS |
| | | KLAAKWPTKLVKNCFLPLREYFKYFSTELTSSL |
| 580 | mouse DNMT3L | MGSRETPSSCSKTLETLDLETSDSSSPDADSPLEEQWLKSSPALK |
| | | EDSVDVVLEDCKEPLSPSSPPTGREMIRYEVKVNRRSIEDICLCC |
| | | GTLQVYTRHPLFEGGLCAPCKDKFLESLFLYDDDGHQSYCTICCS |
| | | GGTLFICESPDCTRCYCFECVDILVGPGTSERINAMACWVCFLCL |
| | | PFSRSGLLQRRKRWRHQLKAFHDQEGAGPMEIYKTVSAWKRQPVR |
| | | VLSLFRNIDKVLKSLGFLESGSGSGGGTLKYVEDVINVVRRDVEK |
| | | WGPFDLVYGSTQPLGSSCDRCPGWYMFQFHRILQYALPRQESQRP |
| | | FFWIFMDNLLLTEDDQETTTRFLQTEAVTLQDVRGRDYQNAMRVW |
| | | SNIPGLKSKHAPLTPKEEEYLQAQVRSRSKLDAPKVDLLVKNCLL |
| | | PLREYFKYFSQNSLPL |
| 581 | mouse DNMT3L | GPMEIYKTVSAWKRQPVRVLSLFRNIDKVLKSLGFLESGSGSGGG |
| | catalytic domain | TLKYVEDVTNVVRRDVEKWGPFDLVYGSTQPLGSSCDRCPGWYMF |
| | | QFHRILQYALPRQESQRPFFWIFMDNLLLTEDDQETTTRFLQTEA |
| | | VTLQDVRGRDYQNAMRVWSNIPGLKSKHAPLTPKEEEYLQAQVRS |
| | | RSKLDAPKVDLLVKNCLLPLREYFKYFSQNSLPL |
| 582 | Ailuropoda | MALSPTGTLSVETLDRSDPDPLDEGPWQATCEILLEPDAEHSTDV |
| | melanoleuca | ILVGSSELSAPASPGPRRDLLAYEVKVNQRDIEDVCICCGSLRVH |
| | DNMT3L | TQHPLFEGGMCAPCKDKFLDCLFLYDDDGYQSYCSICCAGETLLI |
| | | CENPDCTRPSLMMKLRLFRECACLIFPSEGMLLQTVWFWKMTVVW |
| | | QPGLRHLPQENPLETYKTVPVWKREPVRVLSLFGDIRRELMSLGF |
| | | LESGSAPGRLKHLDDVTDVVRKDVEGWGPFDLVYGSTPPIGHACD |
| | | HPPVWYLLQFHRILQYARPRPGSQQPFFWMFVDNLVLSQDDQTAA |
| | | TRFLEADPVTIQDVCGRAVRNTVHVWSNIPAVRSRHSALALCEEL |
| | | SLLAQDRQRTKPPAQGPAQLVKNCFLPLREYFKYFSTELTSSL |
| 583 | Ailuropoda | NPLETYKTVPVWKREPVRVLSLFGDIRRELMSLGFLESGSAPGRL |
| | melanoleuca | KHLDDVTDVVRKDVEGWGPFDLVYGSTPPIGHACDHPPVWYLLQF |
| | DNMT3L catalytic | HRILQYARPRPGSQQPFFWMFVDNLVLSQDDQTAATRFLEADPVT |
| | domain | IQDVCGRAVRNTVHVWSNIPAVRSRHSALALCEELSLLAQDRQRT |
| | | KPPAQGPAQLVKNCFLPLREYFKYFSTELTSSL |
| 584 | Carlito syrichta | MALSCRRTLPLESLHSSNSDLASQLDKEQWRPPCETHGIPVAAAP |
| | DNMT3L | VLDLEAECSLDVILVGSSELSTSSSPRLGRDHIAYEVKVNQRNIE |
| | | DICLCCGSFLVHTQHPLFEGGMCAPCKDKFLDTLFLYDEDGYQSY |
| | | CSICCSGETLLICENPDCTRCYCFECLDILVSPGTSEKVHAMSNW |
| | | VCFLCLPFTRSGLLQRRRKWRGQLKAFYDRESESSLEMYKTVPVW |
| | | KREPVRVLSLFGDIKKELMSLGFVETGSDPGRLRHLDDTTNIVRR |
| | | NVEEWGPFHLLYGATPPLGHTCDRPPGWYLFQFHRLLQYARPQPG |
| | | SPQPFFWMFVDNVMLTREDRAIASRFLETEPVTIPDIHGRALQNA |
| | | VCVWSNIPAVRSKHSALVSEEELSLLAQDRQRAKLPTQGPTKLVK |
| | | NCFLPLREYFKYFSTELTSFL |
| 585 | Carlito syrichta | SSLEMYKTVPVWKREPVRVLSLFGDIKKELMSLGFVETGSDPGRL |
| | DNMT3L catalytic | RHLDDTTNIVRRNVEEWGPFHLLYGATPPLGHTCDRPPGWYLFQF |
| | domain | HRLLQYARPQPGSPQPFFWMFVDNVMLTREDRAIASRFLETEPVT |
| | | IPDIHGRALQNAVCVWSNIPAVRSKHSALVSEEELSLLAQDRQRA |
| | | KLPTQGPTKLVKNCFLPLREYFKYFSTELTSFL |
| 586 | Meriones | MGSQETPSTRAKTPGTWNLESTDSSSPESLGHLEEQWANSSPDLK |
| | unguiculatus | DEHSKDVEPEDSKELISSASPPSGREIIRYEISVNQRNIEDICLC |
| | DNMT3L | CGTLQVYKQHPLFEGGICAPCKDKFLETFFLYDEDGHQSYCSICC |
| | | SGGTLFICESPDCTRCYCFECVDILVGPGTSERINAMPCWVCFLC |
| | | LPFTRSGLLQRRRKWRHQLKAFFDEGGASPLEMYKTVSAWKRKPM |
| | | RVLSLFKNIDKELKNLGFLESGSGSEEERLKYLEDVINVVRRDVE |
| | | KWGPFDLVYGSTRPRGSSCDHCPAWYMFQFHRILQYARPPSGSEQ |
| | | PFFWVFVDNLLMTEDDQITADRFLQMKAVTLQDVRGRVLQNAVRV |
| | | WSNIPGVKSKHMALTEKEEQSLEAQAGTRTKLSAQKVDPLVKNCL |
| | | LPLREYFKFFSQNSLPLDK |
| 587 | Meriones | SPLEMYKTVSAWKRKPMRVLSLFKNIDKELKNLGFLESGSGSEEE |
| | unguiculatus | RLKYLEDVINVVRRDVEKWGPFDLVYGSTRPRGSSCDHCPAWYMF |
| | DNMT3L catalytic | QFHRILQYARPPSGSEQPFFWVFVDNLLMTEDDQITADRFLQMKA |
| | domain | VTLQDVRGRVLQNAVRVWSNIPGVKSKHMALTEKEEQSLEAQAGT |
| | | RTKLSAQKVDPLVKNCLLPLREYFKFFSQNSLPLDK |
| 588 | Ochotona princeps | MALPSPETLDSLDRVPASHPDEQHWTVCDNSDPILEVEAEGSMDV |
| | DNMT3L | ILVDDSPAPSGRDRIELEVKVNQRSIEDLCLCCGSSQVHRQHPLF |
| | | QGGLCAPCKDKFLEALFLYDEDGYQSYCSICGLGDTLLVCESPDC |
| | | TRGYCFACVDGLVGAGSSGHMHTVSPWVCFLCVPGSRHGLLQRRR |
| | | RWRTQLKVFHEQEAAQPLEIYETVPACRRKPLRVLSLFEHIEKEL |
| | | ASLGFLETGSSPGRIRHLDDVTDVVRRDVEQWGPFDLVYGSTPPL |
| | | GHASPRSPGWYLFQFHRMLQYTQPTASTQRPFFWMFVDNLLLTRD |
| | | DLVTATRFLEVEPATLQDVRGRVLQGAMRVWSNIPAVNSRHTELA |
| | | PEAETALLAQSCRRAKASGEGLARLLKSCFLPLREYFKYFPQSPL |
| | | PLRK |
| 589 | Ochotona princeps | QPLEIYETVPACRRKPLRVLSLFEHIEKELASLGFLETGSSPGRI |
| | DNMT3L catalytic | RHLDDVTDVVRRDVEQWGPFDLVYGSTPPLGHASPRSPGWYLFQF |
| | domain | HRMLQYTQPTASTQRPFFWMFVDNLLLTRDDLVTATRFLEVEPAT |
| | | LQDVRGRVLQGAMRVWSNIPAVNSRHTELAPEAETALLAQSCRRA |
| | | KASGEGLARLLKSCFLPLREYFKYFPQSPLPLRK |
| 590 | Neosciurus | MGGPRPAAVEESPHEIYKTVPAWKREPMRVLSLFGDIGKELTSLG |
| | carolinensis | FLETGSEAGRLKHLEDVTDTVRRDVEEWGPFDLVYGSTPALGHSC |
| | DNMT3L | DRSPGWYLFQFHRLLQYARPRLGSPKPFFWMFVDNLLLTKDDQAI |
| | | ASRFLEMEPVTLQDVHGRVLQNAVRVWTNVPAVKSRHSALASEEE |
| | | LLLVQDGQRGRLPAQGPAALVKHCFLPLREYFKYFSQNTLPLYK |
| 591 | Neosciurus | SPHEIYKTVPAWKREPMRVLSLFGDIGKELTSLGFLETGSEAGRL |
| | carolinensis | KHLEDVTDTVRRDVEEWGPFDLVYGSTPALGHSCDRSPGWYLFQF |
| | DNMT3L catalytic | HRLLQYARPRLGSPKPFFWMFVDNLLLIKDDQAIASRFLEMEPVT |
| | domain | LQDVHGRVLQNAVRVWTNVPAVKSRHSALASEEELLLVQDGQRGR |
| | | LPAQGPAALVKHCFLPLREYFKYFSQNTLPLYK |
| 592 | Bison bison | MARSSPGTLNLEIMDGSDPDPALPPDREQWPPPCEILLDPEPEHS |
| | DNMT3L | LDIILVGSSELSSPPSPGPRRDFIAYEVKVNQRDIEDVCICCGSL |
| | | QLHTQHPLFEGGMCAPCKDKFLECLFLYDDDGYQSYCSICCAGET |
| | | LLICENPDCTRCYCFECVDTLVGPGTSGKVHAMSNWVCFLCLPFP |
| | | RSGLLQRRRKWRTWLKAFYDREAESPLVMYKTVPVWKREPIRVLS |
| | | LFGDIKKELTSLGFLEDGSKPGRLKHLDDVTNIVRRDIDEWGPED |
| | | LTYGSTPTLGHTCDHPPGWYVYQFHRILQYARPLPGSPQPFFWMF |
| | | VDNLVLTEEDLDVATRFLETDPVTIQDVRGRTVQNAVHVWSNIPA |
| | | VKSRHSALVSQEELSLLAQDRQRVKSPVQGPATLVKNCFLPLREY |
| | | FKYFSTELTSSL |
| 593 | Bison bison | SPLVMYKTVPVWKREPIRVLSLFGDIKKELTSLGFLEDGSKPGRL |
| | DNMT3L catalytic | KHLDDVTNIVRRDIDEWGPFDLTYGSTPTLGHTCDHPPGWYVYQF |
| | domain | HRILQYARPLPGSPQPFFWMFVDNLVLTEEDLDVATRFLETDPVT |
| | | IQDVRGRTVQNAVHVWSNIPAVKSRHSALVSQEELSLLAQDRQRV |
| | | KSPVQGPATLVKNCFLPLREYFKYFSTELTSSL |
| 594 | Equus przewalskii | MALSSPGTLSLETLDSWDPDVAGQLDEERWQPSSEIVGRPMAAAP |
| | DNMT3L | VLDLEEEPSMDIILVDSSELSSPPSPGPSRDMCICCGSFQVHTQH |
| | | PLFEGGMCAACKDKFLSCLFLYDDDGNQSYCSICCSGETLLICEN |
| | | PDCTRCYCFECVDTLVSPRISEKVQAMSNWVCFLCLPFPRSGLLQ |
| | | RRRKWRGWLKAFYDQEAVRSRSAWGRRMRSGPHLVGFLWLLVAKC |
| | | PSALESPLEMYKTVPVWKREPVRVLSLFGDIKKELTTLGFLENGS |
| | | DPGRLKHLDDVTNTVRRDVEEWGPFDLVYGSTPPLGHACDHPPGW |
| | | YLFQFHRVLQYARPRPGSPQAFFWMFVDNLVLTEDDRAVATRFLE |
| | | TDPVTIQDVCGRAVRNAVHVWSNIPAVKSRHSALFSQEESFLRAQ |
| | | DRQRAKPPARGPAKLVKNCFLPLREYFKYFSTEFTSSL |
| 595 | Equus przewalskii | SPLEMYKTVPVWKREPVRVLSLFGDIKKELTTLGFLENGSDPGRL |
| | DNMT3L catalytic | KHLDDVTNTVRRDVEEWGPFDLVYGSTPPLGHACDHPPGWYLFQF |
| | domain | HRVLQYARPRPGSPQAFFWMFVDNLVLTEDDRAVATRFLETDPVT |
| | | IQDVCGRAVRNAVHVWSNIPAVKSRHSALFSQEESFLRAQDRQRA |
| | | KPPARGPAKLVKNCFLPLREYFKYFSTEFTSSL |
| 596 | Mus caroli | MGSRETPSSFSKTLETLDLETSDSSSPDADSPLEEQWLKSSPALK |
| | DNMT3L | EDNVDMVLEDCKEPLSPSSPPTGREMIRYEVKVNRRSIEDICLCC |
| | | GTLQVYTQHPLFEGGICAPCKDKFLESLFLYDDDGHQSYCTICCS |
| | | GGTLFICESPDCTRCYCFECVDILVGPGTSERINAMACWVCFLCL |
| | | PFSRSGLLQRRKRWRHQLKAFHDQEGAGPMEIYKTVSTWKRQPVR |
| | | VLSLFGNIDKVLKSLGFLESGSGSGGGTLKYVEDVINVVRRDVEK |
| | | WGPFDLVYGSTQPLGSSCDRCPGWYMFQFHRILQYALPRQESQRP |
| | | FFWIFMDNLLMTEDDQETTARFLQTEAVTLQDVRGRDYQNVMRVW |
| | | SNIPGLKSKHVPLTPKEEEYLQAQVRTRSKLDAQKVDLLVKNCLL |
| | | PLREYFKYFS |
| 597 | Mus caroli | GPMEIYKTVSTWKRQPVRVLSLFGNIDKVLKSLGFLESGSGSGGG |
| | DNMT3L catalytic | TLKYVEDVTNVVRRDVEKWGPFDLVYGSTQPLGSSCDRCPGWYMF |
| | domain | QFHRILQYALPRQESQRPFFWIFMDNLLMTEDDQETTARFLQTEA |
| | | VTLQDVRGRDYQNVMRVWSNIPGLKSKHVPLTPKEEEYLQAQVRT |
| | | RSKLDAQKVDLLVKNCLLPLREYFKYFS |
| 598 | Pan troglodytes | MAAIPALDPEAEPSMDVILVGSSELSSSISPRTGRDLIAYEVKAN |
| | DNMT3L | QRNIEDICICCGSLQVHTQHPLFEGGICAPCKDKSLDALFLYDDD |
| | | GYQSYCSICCSGETLLICGNPDCTRCYCFECVDSLVGPGTSGKVH |
| | | AMSNWVCYLCLPSSRSGLLQRRRKWRSQLKAFYDRESENPLEMFE |
| | | TVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYA |
| | | RPKPGSPRPFFWMFVDNLVLNKEDLDVASRFLEMEPVTIPDVHGG |
| | | SLQNAVRVWSNIPAIRSSRHWALVSEEELSLLAQNKQSSKLAAKW |
| | | PTKLVKNCFLPLREYFKYFSTELTSSL |
| 599 | Pan troglodytes | NPLEMFETVPVWRRQPVRVLSLFEDIKKELTSLGFLESGSDPGQL |
| | DNMT3L catalytic | KHVVDVTDTVRKDVEEWGPFDLVYGATPPLGHTCDRPPSWYLFQF |
| | domain | HRLLQYARPKPGSPRPFFWMFVDNLVLNKEDLDVASRFLEMEPVT |
| | | IPDVHGGSLQNAVRVWSNIPAIRSSRHWALVSEEELSLLAQNKQS |
| | | SKLAAKWPTKLVKNCFLPLREYFKYFSTELTSSL |
| 600 | human TRDMT1 | MEPLRVLELYSGVGGMHHALRESCIPAQVVAAIDVNIVANEVYKY |
| | (DNMT2) | NFPHTQLLAKTIEGITLEEFDRLSFDMILMSPPCQPFTRIGRQGD |
| | | MTDSRTNSFLHILDILPRLQKLPKYILLENVKGFEVSSTRDLLIQ |
| | | TIENCGFQYQEFLLSPTSLGIPNSRLRYFLIAKLQSEPLPFQAPG |
| | | QVLMEFPKIESVHPQKYAMDVENKIQEKNVEPNISFDGSIQCSGK |
| | | DAILFKLETAEEIHRKNQQDSDLSVKMLKDFLEDDTDVNQYLLPP |
| | | KSLLRYALLLDIVQPTCRRSVCFTKGYGSYIEGTGSVLQTAEDVQ |
| | | VENIYKSLTNLSQEEQITKLLILKLRYFTPKEIANLLGFPPEFGF |
| | | PEKITVKQRYRLLGNSLNVHVVAKLIKILYE |
| 601 | M. bacterium | MAEWYIPAIVSYQAIHNGFTLNKINHKIELQTMIDYLESKTLSMN |
| | methyltransferase | SKEPVKRGFWYKKHLDEIRIVYTAVKMSEQEGNIFDVRTLFERGL |
| | | SDIDLLTYSFPCQDLSQQGKQKGMGRDSQTRSGLLWEIEKALDTS |
| | | KKEDLPKYLLMENVVALTHKVNAEELDEWMMKLESLGYKNDLRIL |
| | | NAGDFGSSQARRRTFMISTLNEKVELPVGNKKPKSMNKILNDEPT |
| | | RKDFLPALDKFDLTEYKWTKSNINKAKLINYSTENSEAYVYDSNF |
| | | TGPTLTASGANSRIKFEYNGKIRKIGAEEAYAYMGFKKSDYIKVN |
| | | KLNYLNETKMIYTCGNSISVEVLRSIMTNINNNFKENK |
| 602 | M. marinum | MLFLIGTFKYVLIYITKVIRIFEAFAGIGAQRKALRNIKSNYEVS |
| | methyltransferase | GMAEWYIPAIVSYQAIHNGFTLSRVDKKTKLTEMIKYLESKTLSM |
| | | DSKEPVRTGYWFKKHKDMVRIVYSAVKLSEAEGNIFDVRTLHERK |
| | | LEDIDLLTYSFPCQDLSQQGKQRGMKKDSGTRSGLLWEIEKALEA |
| | | TPKDKLPKYLLMENVVALTHKTNKKDLDNWKRKLRSLGYYNDINV |
| | | LNAGDFGSSQARRRAFMISTLDSKVTLPLGDKKPQAISKILNKET |
| | | RSQDEMPALDEYEKTDFKRTLSNIKKCKLIDYTSFNSEAYVYDPK |
| | | YTGPTLTASGANSRIKFTHQGKMRKINAEEAYRYMGFSTNDYKKV |
| | | NNLNFLSETKMIYTCGNSISVEVLEEIMLKIIREDNNG |
| 603 | S. chinense | MKKIRLFEAFAGIGSQRRALKSVVGNNFEIAGLAEWYVPAIVMYQ |
| | methyltransferase | IINNDFSKKNVLDNVPRDEVIDYLNSKCLSWDSKKPVSKNFWNRK |
| | | SQDILNVIYSAVKKSEEEGNIFDVRTLHERTLESIDILTYSFPCQ |
| | | DLSQQGIQKGMKKNSGTRSGLLWEIEKAIDNTPKNNLPKILLMEN |
| | | VPALLNKTNELELKEWLIKLENMGYKNSIGILNAADFGSPQARRR |
| | | VFMISSRNKKIELPVGKSKPGKLNDILEKNVEDKFIMTNLEKYDF |
| | | SEFSLIKSNIKKCSLINYTKFNSEAYVYDPDFTGPTLTASGANSR |
| | | IKIYDKGFIRRMSPLESFRYMGFDDEDYKKIDEFEFLTDTQKIFV |
| | | CGNSISIEVLKAIFERIDSNE |
| 604 | M. penetrans | MNSNKDKIKVIKVFEAFAGIGSQFKALKNIARSKNWEIQHSGMVE |
| | M MpeI | WFVDAIVSYVAIHSKNFNPKIEQLDKDILSISNDSKMPISEYGIK |
| | | KINNTIKASYLNYAKKHENNLFDIKKVNKDNFPKNIDIFTYSFPC |
| | | QDLSVQGLQKGIDKELNTRSGLLWEIERILEEIKNSFSKEEMPKY |
| | | LLMENVKNLLSHKNKKNYNTWLKQLEKFGYKSKTYLLNSKNFDNC |
| | | QNRERVFCLSIRDDYLEKTGFKFKELEKVKNPPKKIKDILVDSSN |
| | | YKYLNLNKYETTTFRETKSNIISRSLKNYTTFNSENYVYNINGIG |
| | | PTLTASGANSRIKIETQQGVRYLTPLECFKYMQFDVNDFKKVQST |
| | | NLISENKMIYIAGNSIPVKILEAIFNTLEFVNNEE |
| 605 | S. monobiae | MSKVENKTKKLRVFEAFAGIGAQRKALEKVRKDEYEIVGLAEWYV |
| | M SssI | PAIVMYQAIHNNFHTKLEYKSVSREEMIDYLENKTLSWNSKNPVS |
| | | NGYWKRKKDDELKIIYNAIKLSEKEGNIFDIRDLYKRILKNIDLL |
| | | TYSFPCQDLSQQGIQKGMKRGSGTRSGLLWEIERALDSTEKNDLP |
| | | KYLLMENVGALLHKKNEEELNQWKQKLESLGYQNSIEVLNAADFG |
| | | SSQARRRVFMISTLNEFVELPKGDKKPKSIKKVLNKIVSEKDILN |
| | | NLLKYNLTEFKKTKSNINKASLIGYSKFNSEGYVYDPEFTGPTLT |
| | | ASGANSRIKIKDGSNIRKMNSDETFLYIGFDSQDGKRVNEIEFLT |
| | | ENQKIFVCGNSISVEVLEAIIDKIGG |
| 606 | H. parainfluenzae | MKDVLDDNLLEEPAAQYSLFEPESNPNLREKFTFIDLFAGIGGER |
| | M HpaII | IAMQNLGGKCIFSSEWDEQAQKTYEANFGDLPYGDITLEETKAFI |
| | | PEKFDILCAGFPCQAFSIAGKRGGFEDTRGTLFFDVAEIIRRHQP |
| | | KAFFLENVKGLKNHDKGRTLKTILNVLREDLGYFVPEPAIVNAKN |
| | | FGVPQNRERIYIVGFHKSTGVNSFSYPEPLDKIVTFADIREEKTV |
| | | PTKYYLSTQYIDTLRKHKERHESKGNGFGYEIIPDDGIANAIVVG |
| | | GMGRERNLVIDHRITDFTPTTNIKGEVNREGIRKMTPREWARLQG |
| | | FPDSYVIPVSDASAYKQFGNSVAVPAIQATGKKILEKLGNLYD |
| 607 | A. luteus | MSKANAKYSFVDLFAGIGGFHAALAATGGVCEYAVEIDREAAAVY |
| | M AluI | ERNWNKPALGDITDDANDEGVTLRGYDGPIDVLTGGFPCQPFSKS |
| | | GAQHGMAETRGTLFWNIARIIEEREPTVLILENVRNLVGPRHRHE |
| | | WLTIIETLRFFGYEVSGAPAIFSPHLLPAWMGGTPQVRERVFITA |
| | | TLVPERMRDERIPRTETGEIDAEAIGPKPVATMNDRFPIKKGGTE |
| | | LFHPGDRKSGWNLLISGIIREGDPEPSNVDLRLTETETLWIDAWD |
| | | DLESTIRRATGRPLEGFPYWADSWTDFRELSRLVVIRGFQAPERE |
| | | VVGDRKRYVARTDMPEGFVPASVTRPAIDETLPAWKQSHLRRNYD |
| | | FFERHFAEVVAWAYRWGVYTDLFPASRRKLEWQAQDAPRLWDTVM |
| | | HFRPSGIRAKRPTYLPALVAITQTSIVGPLERRLSPRETARLQGL |
| | | PEWFDFGEQRAAATYKQMGNGVNVGVVRHILREHVRRDRALLKLT |
| | | PAGQRIINAVLADEPDATVGALGAAE |
| 608 | H. aegyptius | MNLISLFSGAGGLDLGFQKAGFRIICANEYDKSIWKTYESNHSAK |
| | M HaeIII | LIKGDISKISSDEFPKCDGIIGGPPCQSWSEGGSLRGIDDPRGKL |
| | | FYEYIRILKQKKPIFFLAENVKGMMAQRHNKAVQEFIQEFDNAGY |
| | | DVHIILLNANDYGVAQDRKRVFYIGFRKELNINYLPPIPHLIKPT |
| | | FKDVIWDLKDNPIPALDKNKTNGNKCIYPNHEYFIGSYSTIFMSR |
| | | NRVRQWNEPAFTVQASGRQCQLHPQAPVMLKVSKNLNKFVEGKEH |
| | | LYRRLIVRECARVQGFPDDFIFHYESLNDGYKMIGNAVPVNLAYE |
| | | IAKTIKSALEICKGN |
| 609 | H. haemolyticus | MIEIKDKQLTGLRFIDLFAGLGGFRLALESCGAECVYSNEWDKYA |
| | M HhaI | QEVYEMNFGEKPEGDITQVNEKTIPDHDILCAGFPCQAFSISGKQ |
| | | KGFEDSRGTLFFDIARIVREKKPKVVFMENVKNFASHDNGNTLEV |
| | | VKNTMNELDYSFHAKVLNALDYGIPQKRERIYMICFRNDLNIQNE |
| | | QFPKPFELNTFVKDLLLPDSEVEHLVIDRKDLVMINQEIEQTTPK |
| | | TVRLGIVGKGGQGERIYSTRGIAITLSAYGGGIFAKTGGYLVNGK |
| | | TRKLHPRECARVMGYPDSYKVHPSTSQAYKQFGNSVVINVLQYIA |
| | | YNIGSSLNFKPY |
| 610 | Moraxella | MKPEILKLIRSKLDLTQKQASEIIEVSDKTWQQWESGKTEMHPAY |
| | M MspI | YSFLQEKLKDKINFEELSAQKTLQKKIFDKYNQNQITKNAEELAE |
| | | ITHIEERKDAYSSDFKFIDLFSGIGGIRQSFEVNGGKCVFSSEID |
| | | PFAKFTYYTNFGVVPFGDITKVEATTIPQHDILCAGFPCQPFSHI |
| | | GKREGFEHPTQGTMFHEIVRIIETKKTPVLFLENVPGLINHDDGN |
| | | TLKVIIETLEDMGYKVHHTVLDASHFGIPQKRKRFYLVAFLNQNI |
| | | HFEFPKPPMISKDIGEVLESDVTGYSISEHLQKSYLFKKDDGKPS |
| | | LIDKNTTGAVKTLVSTYHKIQRLIGTFVKDGETGIRLLTTNECKA |
| | | IMGFPKDFVIPVSRTQMYRQMGNSVVVPVVTKIAEQISLALKIVN |
| | | QQSPQENFELELV |
| 611 | Ascobolus | MSERRYEAGMTVALHEGSFLKIQRVYIRQYHADNRREHMLVGPLF |
| | Masc1 | RRTKYLKALSKKVNEVAIVHESIHVPVQDVIGVRELIITNRPFPE |
| | | CRKGDEHTGRLVCRWVYNLDERAKGREYKKQRYIRRITEAEADPE |
| | | YRVEDRVLRRRWFQEGYIGDEISYKEHGNGDIVDIRSESPLQVLD |
| | | GWGGDLVDLENGEETSIPGPCRSASSYGRLMKPPLAQAADSNTSR |
| | | KYTFGDTFCGGGGVSLGARQAGLEVKWAFDMNPNAGANYRRNFPN |
| | | TDFFLAEAEQFIQLSVGISQHVDILHLSPPCQTFSRAHTIAGKND |
| | | ENNEASFFAVVNLIKAVRPRLFTVEETDGIMDRQSRQFIDTALMG |
| | | ITELGYSFRICVLNAIEYGVCQNRKRLIIIGAAPGEELPPFPLPT |
| | | HQDFFSKDPRRDLLPAVILDDALSTITPESTDHHLNHVWQPAEWK |
| | | TPYDAHRPFKNAIRAGGGEYDIYPDGRRKFTVRELACIQGFPDEY |
| | | EFVGTLTDKRRIIGNAVPPPLSAAIMSTLRQWMTEKDFERME |
| 612 | Arabidopsis MET1 | MVENGAKAAKRKKRPLPEIQEVEDVPRTRRPRRAAACTSFKEKSI |
| | | RVCEKSATIEVKKQQIVEEEFLALRLTALETDVEDRPTRRLNDFV |
| | | LFDSDGVPQPLEMLEIHDIFVSGAILPSDVCTDKEKEKGVRCTSF |
| | | GRVEHWSISGYEDGSPVIWISTELADYDCRKPAASYRKVYDYFYE |
| | | KARASVAVYKKLSKSSGGDPDIGLEELLAAVVRSMSSGSKYFSSG |
| | | AAIIDFVISQGDFIYNQLAGLDETAKKHESSYVEIPVLVALREKS |
| | | SKIDKPLQRERNPSNGVRIKEVSQVAESEALTSDQLVDGTDDDRR |
| | | YAILLQDEENRKSMQQPRKNSSSGSASNMFYIKINEDEIANDYPL |
| | | PSYYKTSEEETDELILYDASYEVQSEHLPHRMLHNWALYNSDLRF |
| | | ISLELLPMKQCDDIDVNIFGSGVVTDDNGSWISLNDPDSGSQSHD |
| | | PDGMCIFLSQIKEWMIEFGSDDIISISIRTDVAWYRLGKPSKLYA |
| | | PWWKPVLKTARVGISILTFLRVESRVARLSFADVTKRLSGLQAND |
| | | KAYISSDPLAVERYLVVHGQIILQLFAVYPDDNVKRCPFVVGLAS |
| | | KLEDRHHTKWIIKKKKISLKELNLNPRAGMAPVASKRKAMQATTT |
| | | RLVNRIWGEFYSNYSPEDPLQATAAENGEDEVEEEGGNGEEEVEE |
| | | EGENGLTEDTVPEPVEVQKPHTPKKIRGSSGKREIKWDGESLGKT |
| | | SAGEPLYQQALVGGEMVAVGGAVTLEVDDPDEMPAIYFVEYMFES |
| | | TDHCKMLHGRFLQRGSMTVLGNAANERELFLINECMTTQLKDIKG |
| | | VASFEIRSRPWGHQYRKKNITADKLDWARALERKVKDLPTEYYCK |
| | | SLYSPERGGFFSLPLSDIGRSSGFCTSCKIREDEEKRSTIKLNVS |
| | | KTGFFINGIEYSVEDFVYVNPDSIGGLKEGSKTSFKSGRNIGLRA |
| | | YVVCQLLEIVPKESRKADLGSFDVKVRRFYRPEDVSAEKAYASDI |
| | | QELYFSQDTVVLPPGALEGKCEVRKKSDMPLSREYPISDHIFFCD |
| | | LFFDTSKGSLKQLPANMKPKFSTIKDDTLLRKKKGKGVESEIESE |
| | | IVKPVEPPKEIRLATLDIFAGCGGLSHGLKKAGVSDAKWAIEYEE |
| | | PAGQAFKQNHPESTVFVDNCNVILRAIMEKGGDQDDCVSTTEANE |
| | | LAAKLTEEQKSTLPLPGQVDFINGGPPCQGFSGMNRFNQSSWSKV |
| | | QCEMILAFLSFADYFRPRYFLLENVRTFVSFNKGQTFQLTLASLL |
| | | EMGYQVRFGILEAGAYGVSQSRKRAFIWAAAPEEVLPEWPEPMHV |
| | | FGVPKLKISLSQGLHYAAVRSTALGAPFRPITVRDTIGDLPSVEN |
| | | GDSRINKEYKEVAVSWFQKEIRGNTIALTDHICKAMNELNLIRCK |
| | | LIPTRPGADWHDLPKRKVTLSDGRVEEMIPFCLPNTAERHNGWKG |
| | | LYGRLDWQGNFPTSVTDPQPMGKVGMCFHPEQHRILIVRECARSQ |
| | | GFPDSYEFAGNINHKHRQIGNAVPPPLAFALGRKLKEALHLKKSP |
| | | QHQP |
| 613 | Ascobolus Masc2 | MELTPELSGVSTDLGGGGSIFAHWRMKEESPAPTEILDDLNVLEW |
| | | EKTTRDYSKEDLRIADQLFSIEDEHQSLPFETADAEDGTPTEEEE |
| | | EKELPMRTLDNFVLYDASDLELAALDLIGTELNIHAVGTVGPIYT |
| | | EGEEDEQEDEDEDVSPPVRIGTQATSASVTQMTVELYIRNIVQYE |
| | | FCFNDDGTVETWIQTTNAHYKLLQPAKCYTSLYRPVNDCLNVITA |
| | | IITLAPESTTMSLKDLLKVMDDKAQAVSYEEVERMSEFIVQHLDQ |
| | | WMETAPKKKSKLIEKSKVYIDLNNLAGIDMVSGVRPPPVRRVTGR |
| | | SSAPKKRIVRNMNDAVLLHQNETTVINWIHQLSAGMFGRALNVLG |
| | | AETADVENLTCDPASAKFVVPQRRLHKRLKWETRGHIPVSEEEYK |
| | | HIYQGKKYAKFFEAVRAVDESKLTIKLGDLVYVLDQDPKVTQTQF |
| | | ATAGREGRKKGAEKEKIQVRFGRVLSIRQPDSNSKDAQNVFIHVQ |
| | | WLVLGCDTILQEMASRRELFLTDSCDTVFADVIYGVAKLTPLGAK |
| | | DIPTVEFHESMATMMGENEFFVRFKYNYQDGSFTDLKDVDAEQIG |
| | | TLQPRVNTHRNPGYCSNCRIKYDNERTGDKWIYENDTEGEPRLER |
| | | SSKGWCIYAQEFVYLQPVEKQPGTTFRVGYISEINKSSVIVELLA |
| | | RVDDDDKSGHISYSDPRHLYFTGTDIKVTFDKIIRKCFVFHDSGD |
| | | QKAKAPLMYGTLQRDLYYYRYEKRKGKAELVPVREIRSIHEQTLN |
| | | DWESRTQIERHGAVSGKKLKGLDIFAGCGGLTLGLDLSGAVDTKW |
| | | DIEFAPSAANTLALNFPDAQVFNQCANVLLSRAIQSEDEGSLDIE |
| | | YDLQGRVLPDLPKKGEVDFIYGGPPCQGFSGVNRYKKGNDIKNSL |
| | | VATFLSYVDHYKPRFVLLENVKGLITTKLGNSKNAEGKWEGGISN |
| | | GVVKFIYRTLISMNYQCRIGLVQSGEYGVPQSRPRVIFLAARMGE |
| | | RLPDLPEPMHAFEVLDSQYALPHIKRYHTTQNGVAPLPRITIGEA |
| | | VSDLPKFQYANPGVWPRHDPYSSAKAQPSDKTIEKFSVSKATSFV |
| | | GYLLQPYHSRPQSEFQRRLRTKLVPSDEPAEKTSLLTTKLVTAHV |
| | | TRLFNKETTQRIVCVPMWPGADHRSLPKEMRPWCLVDPNSQAEKH |
| | | RFWPGLFGRLGMEDFFSTALTDVQPCGKQGKVLHPTQRRVYTVRE |
| | | LARAQGFPDWFAFTDGDADSGLGGVKKWHRNIGNAVPVPLGEQIG |
| | | RCIGYSVWWKDDMIAQLREDGADEDEEMIDGNDQWVEELNTQMAA |
| | | DMPGLPLLVTHLLNLCVYRRLYGPNAKEFLPARVYDKKLEGGRRR |
| | | LVWAML |
| 614 | Neurospora Dim2 | MDSPDRSHGGMFIDVPAETMGFQEDYLDMFASVLSQGLAKEGDYA |
| | | HHQPLPAGKEECLEPIAVATTITPSPDDPQLQLQLELEQQFQTES |
| | | GLNGVDPAPAPESEDEADLPDGFSDESPDDDFVVQRSKHITVDLP |
| | | VSTLINPRSTFQRIDENDNLVPPPQSTPERVAVEDLLKAAKAAGK |
| | | NKEDYIEFELHDENFYVNYAYHPQEMRPIQLVATKVLHDKYYFDG |
| | | VLKYGNTKHYVTGMQVLELPVGNYGASLHSVKGQIWVRSKHNAKK |
| | | EIYYLLKKPAFEYQRYYQPFLWIADLGKHVVDYCTRMVERKREVT |
| | | LGCFKSDFIQWASKAHGKSKAFQNWRAQHPSDDFRISVAANIGYI |
| | | WKEINGVAGAKRAAGDQLFRELMIVKPGQYFRQEVPPGPVVTEGD |
| | | RTVAATIVTPYIKECFGHMILGKVLRLAGEDAEKEKEVKLAKRLK |
| | | IENKNATKADTKDDMKNDTATESLPTPLRSLPVQVLEATPIESDI |
| | | VSIVSSDLPPSENNPPPLINGSVKPKAKANPKPKPSTQPLHAAHV |
| | | KYLSQELVNKIKVGDVISTPRDDSSNTDTKWKPTDTDDHRWFGLV |
| | | QRVHTAKTKSSGRGLNSKSFDVIWFYRPEDTPCCAMKYKWRNELF |
| | | LSNHCTCQEGHHARVKGNEVLAVHPVDWFGTPESNKGEFFVRQLY |
| | | ESEQRRWITLQKDHLTCYHNQPPKPPTAPYKPGDTVLATLSPSDK |
| | | FSDPYEVVEYFTQGEKETAFVRLRKLLRRRKVDRQDAPANELVYT |
| | | EDLVDVRAERIVGKCIMRCFRPDERVPSPYDRGGIGNMFFITHRQ |
| | | DHGRCVPLDTLPPTLRQGFNPLGNLGKPKLRGMDLYCGGGNFGRG |
| | | LEEGGVVEMRWANDIWDKAIHTYMANTPDPNKTNPFLGSVDDLLR |
| | | LALEGKFSDNVPRPGEVDFIAAGSPCPGFSLLTQDKKVLNQVKNQ |
| | | SLVASFASFVDFYRPKYGVLENVSGIVQTFVNRKQDVLSQLFCAL |
| | | VGMGYQAQLILGDAWAHGAPQSRERVFLYFAAPGLPLPDPPLPSH |
| | | SHYRVKNRNIGFLCNGESYVQRSFIPTAFKFVSAGEGTADLPKIG |
| | | DGKPDACVRFPDHRLASGITPYIRAQYACIPTHPYGMNFIKAWNN |
| | | GNGVMSKSDRDLFPSEGKTRTSDASVGWKRLNPKTLFPTVTITSN |
| | | PSDARMGPGLHWDEDRPYTVQEMRRAQGYLDEEVLVGRTTDQWKL |
| | | VGNSVSRHMALAIGLKFREAWLGTLYDESAVVATATATATTAAAV |
| | | GVTVPVMEEPGIGTTESSRPSRSPVHTAVDLDDSKSERSRSTTPA |
| | | TVLSTSSAAGDGSANAAGLEDDDNDDMEMMEVTRKRSSPAVDEEG |
| | | MRPSKVQKVEVTVASPASRRSSRQASRNPTASPSSKASKATTHEA |
| | | PAPEELESDAESYSETYDKEGEDGDYHSGHEDQYSEEDEEEEYAE |
| | | PETMTVNGMTIVKL |
| 615 | Drosophila dDnmt2 | MVFRVLELFSGIGGMHYAFNYAQLDGQIVAALDVNTVANAVYAHN |
| | | YGSNLVKTRNIQSLSVKEVTKLQANMLLMSPPCQPHTRQGLQRDT |
| | | EDKRSDALTHLCGLIPECQELEYILMENVKGFESSQARNQFIESL |
| | | ERSGFHWREFILTPTQFNVPNTRYRYYCIARKGADFPFAGGKIWE |
| | | EMPGAIAQNQGLSQIAEIVEENVSPDFLVPDDVLTKRVLVMDIIH |
| | | PAQSRSMCFTKGYTHYTEGTGSAYTPLSEDESHRIFELVKEIDTS |
| | | NQDASKSEKILQQRLDLLHQVRLRYFTPREVARLMSFPENFEFPP |
| | | ETTNRQKYRLLGNSINVKVVGELIKLLTIK |
| 616 | S. pombe Pmt1 | MLSTKRLRVLELYSGIGGMHYALNLANIPADIVCAIDINPQANEI |
| | | YNLNHGKLAKHMDISTLTAKDFDAFDCKLWTMSPSCQPFTRIGNR |
| | | KDILDPRSQAFLNILNVLPHVNNLPEYILIENVQGFEESKAAEEC |
| | | RKVLRNCGYNLIEGILSPNQFNIPNSRSRWYGLARLNFKGEWSID |
| | | DVFQFSEVAQKEGEVKRIRDYLEIERDWSSYMVLESVLNKWGHQF |
| | | DIVKPDSSSCCCFTRGYTHLVQGAGSILQMSDHENTHEQFERNRM |
| | | ALQLRYFTAREVARLMGFPESLEWSKSNVTEKCMYRLLGNSINVK |
| | | VVSYLISLLLEPLNF |
| 617 | Arabidopsis DRM1 | MVMSHIFLISQIQEVEHGDSDDVNWNTDDDELAIDNFQFSPSPVH |
| | | ISATSPNSIQNRISDETVASFVEMGFSTQMIARAIEETAGANMEP |
| | | MMILETLFNYSASTEASSSKSKVINHFIAMGFPEEHVIKAMQEHG |
| | | DEDVGEITNALLTYAEVDKLRESEDMNININDDDDDNLYSLSSDD |
| | | EEDELNNSSNEDRILQALIKMGYLREDAAIAIERCGEDASMEEVV |
| | | DFICAAQMARQFDEIYAEPDKKELMNNNKKRRTYTETPRKPNTDQ |
| | | LISLPKEMIGFGVPNHPGLMMHRPVPIPDIARGPPFFYYENVAMT |
| | | PKGVWAKISSHLYDIVPEFVDSKHFCAAARKRGYIHNLPIQNRFQ |
| | | IQPPQHNTIQEAFPLTKRWWPSWDGRIKLNCLLTCIASSRLTEKI |
| | | REALERYDGETPLDVQKWVMYECKKWNLVWVGKNKLAPLDADEME |
| | | KLLGFPRDHTRGGGISTTDRYKSLGNSFQVDTVAYHLSVLKPLFP |
| | | NGINVLSLFTGIGGGEVALHRLQIKMNVVVSVEISDANRNILRSF |
| | | WEQTNQKGILREFKDVQKLDDNTIERLMDEYGGFDLVIGGSPCNN |
| | | LAGGNRHHRVGLGGEHSSLFFDYCRILEAVRRKARHMRR |
| 618 | Arabidopsis DRM2 | MVIWNNDDDDFLEIDNFQSSPRSSPIHAMQCRVENLAGVAVTTSS |
| | | LSSPTETTDLVQMGFSDEVFATLFDMGFPVEMISRAIKETGPNVE |
| | | TSVIIDTISKYSSDCEAGSSKSKAIDHFLAMGFDEEKVVKAIQEH |
| | | GEDNMEAIANALLSCPEAKKLPAAVEEEDGIDWSSSDDDTNYTDM |
| | | LNSDDEKDPNSNENGSKIRSLVKMGFSELEASLAVERCGENVDIA |
| | | ELTDFLCAAQMAREFSEFYTEHEEQKPRHNIKKRRFESKGEPRSS |
| | | VDDEPIRLPNPMIGFGVPNEPGLITHRSLPELARGPPFFYYENVA |
| | | LTPKGVWETISRHLFEIPPEFVDSKYFCVAARKRGYIHNLPINNR |
| | | FQIQPPPKYTIHDAFPLSKRWWPEWDKRTKLNCILTCTGSAQLIN |
| | | RIRVALEPYNEEPEPPKHVQRYVIDQCKKWNLVWVGKNKAAPLEP |
| | | DEMESILGFPKNHTRGGGMSRTERFKSLGNSFQVDTVAYHLSVLK |
| | | PIFPHGINVLSLFTGIGGGEVALHRLQIKMKLVVSVEISKVNRNI |
| | | LKDFWEQTNQTGELIEFSDIQHLINDTIEGLMEKYGGEDLVIGGS |
| | | PCNNLAGGNRVSRVGLEGDQSSLFFEYCRILEVVRARMRGS |
| 619 | Arabidopsis CMT1 | MAARNKQKKRAEPESDLCFAGKPMSVVESTIRWPHRYQSKKTKLQ |
| | | APTKKPANKGGKKEDEEIIKQAKCHFDKALVDGVLINLNDDVYVT |
| | | GLPGKLKFIAKVIELFEADDGVPYCRFRWYYRPEDTLIERFSHLV |
| | | QPKRVFLSNDENDNPLTCIWSKVNIAKVPLPKITSRIEQRVIPPC |
| | | DYYYDMKYEVPYLNFTSADDGSDASSSLSSDSALNCFENLHKDEK |
| | | FLLDLYSGCGAMSTGFCMGASISGVKLITKWSVDINKFACDSLKL |
| | | NHPETEVRNEAAEDFLALLKEWKRLCEKFSLVSSTEPVESISELE |
| | | DEEVEENDDIDEASTGAELEPGEFEVEKFLGIMFGDPQGTGEKTL |
| | | QLMVRWKGYNSSYDTWEPYSGLGNCKEKLKEYVIDGFKSHLLPLP |
| | | GTVYTVCGGPPCQGISGYNRYRNNEAPLEDQKNQQLLVFLDIIDF |
| | | LKPNYVLMENVVDLLRFSKGFLARHAVASFVAMNYQTRLGMMAAG |
| | | SYGLPQLRNRVFLWAAQPSEKLPPYPLPTHEVAKKENTPKEFKDL |
| | | QVGRIQMEFLKLDNALTLADAISDLPPVTNYVANDVMDYNDAAPK |
| | | TEFENFISLKRSETLLPAFGGDPTRRLFDHQPLVLGDDDLERVSY |
| | | IPKQKGANYRDMPGVLVHNNKAEINPRFRAKLKSGKNVVPAYAIS |
| | | FIKGKSKKPFGRLWGDEIVNTVVTRAEPHNQCVIHPMQNRVLSVR |
| | | ENARLQGFPDCYKLCGTIKEKYIQVGNAVAVPVGVALGYAFGMAS |
| | | QGLTDDEPVIKLPFKYPECMQAKDQI |
| 620 | Arabadopsis CMT2 | MLSPAKCESEEAQAPLDLHSSSRSEPECLSLVLWCPNPEEAAPSS |
| | | TRELIKLPDNGEMSLRRSTTLNCNSPEENGGEGRVSQRKSSRGKS |
| | | QPLLMLTNGCQLRRSPRFRALHANFDNVCSVPVTKGGVSQRKFSR |
| | | GKSQPLLTLINGCQLRRSPRFRAVDGNFDSVCSVPVTGKFGSRKR |
| | | KSNSALDKKESSDSEGLIFKDIAVIAKSLEMEIISECQYKNNVAE |
| | | GRSRLQDPAKRKVDSDTLLYSSINSSKQSLGSNKRMRRSQRFMKG |
| | | TENEGEENLGKSKGKGMSLASCSFRRSTRLSGTVETGNTETLNRR |
| | | KDCGPALCGAEQVRGTERLVQISKKDHCCEAMKKCEGDGLVSSKQ |
| | | ELLVFPSGCIKKTVNGCRDRTLGKPRSSGLNTDDIHTSSLKISKN |
| | | DTSNGLTMTTALVEQDAMESLLQGKTSACGAADKGKTREMHVNST |
| | | VIYLSDSDEPSSIEYLNGDNLTQVESGSALSSGGNEGIVSLDLNN |
| | | PTKSTKRKGKRVTRTAVQEQNKRSICFFIGEPLSCEEAQERWRWR |
| | | YELKERKSKSRGQQSEDDEDKIVANVECHYSQAKVDGHTFSLGDF |
| | | AYIKGEEEETHVGQIVEFFKTTDGESYFRVQWFYRATDTIMERQA |
| | | TNHDKRRLFYSTVMNDNPVDCLISKVTVLQVSPRVGLKPNSIKSD |
| | | YYFDMEYCVEYSTFQTLRNPKISENKLECCADVVPTESTESILKK |
| | | KSFSGELPVLDLYSGCGGMSTGLSLGAKISGVDVVTKWAVDQNTA |
| | | ACKSLKLNHPNTQVRNDAAGDFLQLLKEWDKLCKRYVENNDQRTD |
| | | TLRSVNSTKETSGSSSSSDDDSDSEEYEVEKLVDICFGDHDKTGK |
| | | NGLKFKVHWKGYRSDEDTWELAEELSNCQDAIREFVTSGFKSKIL |
| | | PLPGRVGVICGGPPCQGISGYNRHRNVDSPLNDERNQQIIVEMDI |
| | | VEYLKPSYVLMENVVDILRMDKGSLGRYALSRLVNMRYQARLGIM |
| | | TAGCYGLSQFRSRVFMWGAVPNKNLPPFPLPTHDVIVRYGLPLEF |
| | | ERNVVAYAEGQPRKLEKALVLKDAISDLPHVSNDEDREKLPYESL |
| | | PKTDFQRYIRSTKRDLIGSAIDNCNKRTMLLHDHRPFHINEDDYA |
| | | RVCQIPKRKGANFRDLPGLIVRNNTVCRDPSMEPVILPSGKPLVP |
| | | GYVFTFQQGKSKRPFARLWWDETVPTVLTVPTCHSQALLHPEQDR |
| | | VLTIRESARLQGFPDYFQFCGTIKERYCQIGNAVAVSVSRALGYS |
| | | LGMAFRGLARDEHLIKLPQNFSHSTYPQLQETIPH |
| 621 | Arabidopsis CMT3 | MAPKRKRPATKDDTTKSIPKPKKRAPKRAKTVKEEPVTVVEEGEK |
| | | HVARFLDEPIPESEAKSTWPDRYKPIEVQPPKASSRKKTKDDEKV |
| | | EIIRARCHYRRAIVDERQIYELNDDAYVQSGEGKDPFICKIIEMF |
| | | EGANGKLYFTARWFYRPSDTVMKEFEILIKKKRVFFSEIQDTNEL |
| | | GLLEKKLNILMIPLNENTKETIPATENCDFFCDMNYFLPYDTFEA |
| | | IQQETMMAISESSTISSDTDIREGAAAISEIGECSQETEGHKKAT |
| | | LLDLYSGCGAMSTGLCMGAQLSGLNLVTKWAVDMNAHACKSLQHN |
| | | HPETNVRNMTAEDFLFLLKEWEKLCIHFSLRNSPNSEEYANLHGL |
| | | NNVEDNEDVSEESENEDDGEVFTVDKIVGISFGVPKKLLKRGLYL |
| | | KVRWLNYDDSHDTWEPIEGLSNCRGKIEEFVKLGYKSGILPLPGG |
| | | VDVVCGGPPCQGISGHNRFRNLLDPLEDQKNKQLLVYMNIVEYLK |
| | | PKFVLMENVVDMLKMAKGYLARFAVGRLLQMNYQVRNGMMAAGAY |
| | | GLAQFRLRFFLWGALPSEIIPQFPLPTHDLVHRGNIVKEFQGNIV |
| | | AYDEGHTVKLADKLLLKDVISDLPAVANSEKRDEITYDKDPTTPF |
| | | QKFIRLRKDEASGSQSKSKSKKHVLYDHHPLNLNINDYERVCQVP |
| | | KRKGANFRDFPGVIVGPGNVVKLEEGKERVKLESGKTLVPDYALT |
| | | YVDGKSCKPFGRLWWDEIVPTVVTRAEPHNQVIIHPEQNRVLSIR |
| | | ENARLQGFPDDYKLFGPPKQKYIQVGNAVAVPVAKALGYALGTAF |
| | | QGLAVGKDPLLTLPEGFAFMKPTLPSELA |
| 622 | Neurospora Rid | MAEQNPFVIDDEDDVIQIHDEEEVEEEVAEVIDITEDDIEPSELD |
| | | RAFGSRPKEETLPSLLLRDQGFIVRPGMTVELKAPIGRFAISFVR |
| | | VNSIVKVRQAHVNNVTIRGHGFTRAKEMNGMLPKQLNECCLVASI |
| | | DTRDPRP |
| 623 | E. coli strain 12 | MNNNDLVAKLWKLCDNLRDGGVSYQNYVNELASLLFLKMCKETGQ |
| | hsdM | EAEYLPEGYRWDDLKSRIGQEQLQFYRKMLVHLGEDDKKLVQAVF |
| | | HNVSTTITEPKQITALVSNMDSLDWYNGAHGKSRDDFGDMYEGLL |
| | | QKNANETKSGAGQYFTPRPLIKTIIHLLKPQPREVVQDPAAGTAG |
| | | FLIEADRYVKSQTNDLDDLDGDTQDFQIHRAFIGLELVPGTRRLA |
| | | LMNCLLHDIEGNLDHGGAIRLGNTLGSDGENLPKAHIVATNPPFG |
| | | SAAGTNITRIFVHPTSNKQLCFMQHIIETLHPGGRAAVVVPDNVL |
| | | FEGGKGTDIRRDLMDKCHLHTILRLPTGIFYAQGVKINVLFFTKG |
| | | TVANPNQDKNCTDDVWVYDLRINMPSFGKRTPFTDEHLQPFERVY |
| | | GEDPHGLSPRTEGEWSFNAEETEVADSEENKNTDQHLATSRWRKF |
| | | SREWIRTAKSDSLDISWLKDKDSIDADSLPEPDVLAAEAMGELVQ |
| | | ALSELDALMRELGASDEADLQRQLLEEAFGGVKE |
| 624 | E. coli strain 12 | MSAGKLPEGWVIAPVSTVTTLIRGVTYKKEQAINYLKDDYLPLIR |
| | hsdS | ANNIQNGKFDTTDLVFVPKNLVKESQKISPEDIVIAMSSGSKSVV |
| | | GKSAHQHLPFECSFGAFCGVLRPEKLIFSGFIAHFTKSSLYRNKI |
| | | SSLSAGANINNIKPASFDLINIPIPPLAEQKIIAEKLDILLAQVD |
| | | STKARFEQIPQILKRFRQAVLGGAVNGKLTEKWRNFEPQHSVFKK |
| | | LNFESILTELRNGLSSKPNESGVGHPILRISSVRAGHVDQNDIRF |
| | | LECSESELNRHKLQDGDLLFTRYNGSLEFVGVCGLLKKLQHQNLL |
| | | YPDKLIRARLTKDALPEYIEIFFSSPSARNAMMNCVKTTSGQKGI |
| | | SGKDIKSQVVLLPPVKEQAEIVRRVEQLFAYADTIEKQVNNALAR |
| | | VNNLTQSILAKAFRGELTAQWRAENPDLISGENSAAALLEKIKAE |
| | | RAASGGKKASRKKS |
| 625 | T. aquaticus | MGLPPLLSLPSNSAPRSLGRVETPPEVVDFMVSLAEAPRGGRVLE |
| | M TaqI | PACAHGPFLRAFREAHGTAYRFVGVEIDPKALDLPPWAEGILADF |
| | | LLWEPGEAFDLILGNPPYGIVGEASKYPIHVFKAVKDLYKKAFST |
| | | WKGKYNLYGAFLEKAVRLLKPGGVLVFVVPATWLVLEDFALLREF |
| | | LAREGKTSVYYLGEVFPQKKVSAVVIRFQKSGKGLSLWDTQESES |
| | | GFTPILWAEYPHWEGEIIRFETEETRKLEISGMPLGDLFHIRFAA |
| | | RSPEFKKHPAVRKEPGPGLVPVLTGRNLKPGWVDYEKNHSGLWMP |
| | | KERAKELRDFYATPHLVVAHTKGTRVVAAWDERAYPWREEFHLLP |
| | | KEGVRLDPSSLVQWLNSEAMQKHVRTLYRDFVPHLTLRMLERLPV |
| | | RREYGEHTSPESARNF |
| 626 | E. coli | MKKNRAFLKWAGGKYPLLDDIKRHLPKGECLVEPFVGAGSVFLNT |
| | M EcoDam | DFSRYILADINSDLISLYNIVKMRTDEYVQAARELFVPETNCAEV |
| | | YYQFREEFNKSQDPFRRAVLFLYLNRYGYNGLCRYNLRGEFNVPF |
| | | GRYKKPYFPEAELYHFAEKAQNAFFYCESYADSMARADDASVVYC |
| | | DPPYAPLSATANFTAYHTNSFTLEQQAHLAEIAEGLVERHIPVLI |
| | | SNHDTMLTREWYQRAKLHVVKVRRSISSNGGTRKKVDELLALYKP |
| | | GVVSPAKK |
| 627 | C. crescentus | MKFGPETIIHGDCIEQMNALPEKSVDLIFADPPYNLQLGGDLLRP |
| | M CcrMI | DNSKVDAVDDHWDQFESFAAYDKFTREWLKAARRVLKDDGAIWVI |
| | | GSYHNIFRVGVAVQDLGFWILNDIVWRKSNPMPNFKGTRFANAHE |
| | | TLIWASKSQNAKRYTFNYDALKMANDEVQMRSDWTIPLCTGEERI |
| | | KGADGQKAHPTQKPEALLYRVILSTTKPGDVILDPFFGVGTTGAA |
| | | AKRLGRKFIGIEREAEYLEHAKARIAKVVPIAPEDLDVMGSKRAE |
| | | PRVPFGTIVEAGLLSPGDTLYCSKGTHVAKVRPDGSITVGDLSGS |
| | | IHKIGALVQSAPACNGWTYWHFKTDAGLAPIDVLRAQVRAGMN |
| 628 | C. difficile | MDDISQDNFLLSKEYENSLDVDTKKASGIYYTPKIIVDYIVKKTL |
| | CamA | KNHDIIKNPYPRILDISCGCGNFLLEVYDILYDLFEENIYELKKK |
| | | YDENYWTVDNIHRHILNYCIYGADIDEKAISILKDSLINKKVVND |
| | | LDESDIKINLFCCDSLKKKWRYKFDYIVGNPPYIGHKKLEKKYKK |
| | | FLLEKYSEVYKDKADLYFCFYKKIIDILKQGGIGSVITPRYFLES |
| | | LSGKDLREYIKSNVNVQEIVDFLGANIFKNIGVSSCILTFDKKKT |
| | | KETYIDVFKIKNEDICINKFETLEELLKSSKFEHFNINQRLLSDE |
| | | WILVNKDDETFYNKIQEKCKYSLEDIAISFQGIITGCDKAFILSK |
| | | DDVKLNLVDDKFLKCWIKSKNINKYIVDKSEYRLIYSNDIDNENT |
| | | NKRILDEIIGLYKTKLENRRECKSGIRKWYELQWGREKLFFERKK |
| | | IMYPYKSNENRFAIDYDNNFSSADVYSFFIKEEYLDKFSYEYLVG |
| | | ILNSSVYDKYFKITAKKMSKNIYDYYPNKVMKIRIFRDNNYEEIE |
| | | NLSKQIISILLNKSIDKGKVEKLQIKMDNLIMDSLGI |
| 629 | KAP1 | MAASAAAASAAAASAASGSPGPGEGSAGGEKRSTAPSAAASASAS |
| | | AAASSPAGGGAEALELLEHCGVCRERLRPEREPRLLPCLHSACSA |
| | | CLGPAAPAAANSSGDGGAAGDGTVVDCPVCKQQCFSKDIVENYFM |
| | | RDSGSKAATDAQDANQCCTSCEDNAPATSYCVECSEPLCETCVEA |
| | | HQRVKYTKDHTVRSTGPAKSRDGERTVYCNVHKHEPLVLFCESCD |
| | | TLTCRDCQLNAHKDHQYQFLEDAVRNQRKLLASLVKRLGDKHATL |
| | | QKSTKEVRSSIRQVSDVQKRVQVDVKMAILQIMKELNKRGRVLVN |
| | | DAQKVTEGQQERLERQHWTMTKIQKHQEHILRFASWALESDNNTA |
| | | LLLSKKLIYFQLHRALKMIVDPVEPHGEMKFQWDLNAWTKSAEAF |
| | | GKIVAERPGINSTGPAPMAPPRAPGPLSKQGSGSSQPMEVQEGYG |
| | | FGSGDDPYSSAEPHVSGVKRSRSGEGEVSGLMRKVPRVSLERLDL |
| | | DLTADSQPPVFKVFPGSTTEDYNLIVIERGAAAAATGQPGTAPAG |
| | | TPGAPPLAGMAIVKEEETEAAIGAPPTATEGPETKPVLMALAEGP |
| | | GAEGPRLASPSGSTSSGLEVVAPEGTSAPGGGPGTLDDSATICRV |
| | | CQKPGDLVMCNQCEFCFHLDCHLPALQDVPGEEWSCSLCHVLPDL |
| | | KEEDGSLSLDGADSTGVVAKLSPANQRKCERVLLALFCHEPCRPL |
| | | HQLATDSTFSLDQPGGTLDLTLIRARLQEKLSPPYSSPQEFAQDV |
| | | GRMFKQFNKLTEDKADVQSIIGLQRFFETRMNEAFGDTKFSAVLV |
| | | EPPPMSLPGAGLSSQELSGGPGDGP |
| 630 | MECP2 | MVAGMLGLREEKSEDQDLQGLKDKPLKFKKVKKDKKEEKEGKHEP |
| | | VQPSAHHSAEPAEAGKAETSEGSGSAPAVPEASASPKQRRSIIRD |
| | | RGPMYDDPTLPEGWTRKLKQRKSGRSAGKYDVYLINPQGKAFRSK |
| | | VELIAYFEKVGDTSLDPNDFDFTVTGRGSPSRREQKPPKKPKSPK |
| | | APGTGRGRGRPKGSGTTRPKAATSEGVQVKRVLEKSPGKLLVKMP |
| | | FQTSPGGKAEGGGATTSTQVMVIKRPGRKRKAEADPQAIPKKRGR |
| | | KPGSVVAAAAAEAKKKAVKESSIRSVQETVLPIKKRKTRETVSIE |
| | | VKEVVKPLLVSTLGEKSGKGLKTCKSPGRKSKESSPKGRSSSASS |
| | | PPKKEHHHHHHHSESPKAPVPLLPPLPPPPPEPESSEDPTSPPEP |
| | | QDLSSSVCKEEKMPRGGSLESDGCPKEPAKTQPAVATAATAAEKY |
| | | KHRGEGERKDIVSSSMPRPNREEPVDSRTPVTERVS |
| 631 | linker | SGGS |
| 632 | linker | SGGSSGSETPGTSESATPESSGGS |
| 633 | linker | SGGSSGGSSGSETPGTSESATPESSGGSSGGS |
| 634 | linker | GGSGGSPGSPAGSPTSTEEGTSESATPESGPGTSTEPSEGSAPGS |
| | | PAGSPISTEEGTSTEPSEGSAPGTSTEPSEGSAPGTSESATPESG |
| | | PGSEPATSGGSGGS |
| 635 | G linker | GSGGG |
| 636 | GX4 linker | GGGGSGGGGSGGGGSGGGGS |
| 637 | W linker | SSGNSNANSRGPSFSSGLVPLSLRGSH |
| 638 | XTEN linker | SGSETPGTSESATPES |
| | (XTEN16) | |
| 639 | XTEN linker | SGGSSGGSSGSETPGTSESATPES |
| 640 | XTEN linker | SGGSSGGSSGSETPGTSESATPESSGGSSGGSSGGSSGGS |
| 641 | XTEN linker | SGGSSGGSSGSETPGTSESATPESSGGSSGGSSGGSSGGSSGSET |
| | | PGTSESATPESSGGSSGGS |
| 642 | XTEN linker | PGSPAGSPTSTEEGTSESATPESGPGTSTEPSEGSAPGSPAGSPT |
| | | STEEGTSTEPSEGSAPGTSTEPSEGSAPGTSESATPESGPGSEPA |
| | | TS |
| 643 | XTEN linker | GGPSSGAPPPSGGSPAGSPTSTEEGTSESATPESGPGTSTEPSEG |
| | (XTEN80) | SAPGSPAGSPTSTEEGTSTEPSEGSAPGTSTEPSE |
| 644 | NLS | PKKKRKV |
| 645 | NLS | AVKRPAATKKAGQAKKKKLD |
| 646 | NLS | MSRRRKANPTKLSENAKKLAKEVEN |
| 647 | NLS | PAAKRVKLD |
| 648 | NLS | KLKIKRPVK |
| 649 | NLS | MDSLLMNRRKFLYQFKNVRWAKGRRETYLC |
| 653 | Coding Sequence of | TACAATCACGATCAGGAGTTCGACCCCCCTAAGGTGTACCCACCA |
| | DNA Methylation | GTGCCTGCAGAGAAGAGGAAGCCAATCCGGGTGCTGAGCCTGTTT |
| | Domain | GATGGCATCGCCACCGGCCTGCTGGTGCTGAAGGATCTGGGCATC |
| | | CAGGTGGACCGGTACATCGCCTCCGAGGTGTGCGAGGATTCTATC |
| | | ACCGTGGGCATGGTGCGCCACCAGGGCAAGATCATGTATGTGGGC |
| | | GACGTGCGGTCCGTGACACAGAAGCACATCCAGGAGTGGGGCCCA |
| | | TTCGATCTGGTGATCGGCGGCAGCCCCTGTAATGACCTGTCCATC |
| | | GTGAACCCTGCAAGGAAGGGACTGTACGAGGGAACCGGCCGGCTG |
| | | TTCTTTGAGTTTTATAGACTGCTGCACGACGCCAGGCCTAAGGAG |
| | | GGCGACGATAGACCATTCTTTTGGCTGTTCGAGAATGTGGTGGCT |
| | | ATGGGCGTGAGCGATAAGAGGGACATCTCCAGGTTTCTGGAGTCT |
| | | AACCCCGTGATGATCGATGCAAAGGAGGTGTCCGCCGCACACAGA |
| | | GCCAGGTATTTCTGGGGCAATCTGCCAGGAATGAACAGGCCACTG |
| | | GCAAGCACCGTGAATGACAAGCTGGAGCTGCAGGAGTGCCTGGAG |
| | | CACGGAAGGATCGCCAAGTTTTCCAAGGTGCGCACAATCACCACA |
| | | CGGAGCAATTCCATCAAGCAGGGCAAGGATCAGCACTTCCCCGTG |
| | | TTCATGAACGAGAAGGAGGACATCCTGTGGTGTACCGAGATGGAG |
| | | AGAGTGTTCGGCTTTCCAGTGCACTACACAGACGTGTCTAACATG |
| | | AGCAGGCTGGCAAGGCAGCGGCTGCTGGGCAGATCTTGGAGCGTG |
| | | CCCGTGATCAGGCACCTGTTCGCCCCTCTGAAGGAGTATTTTGCC |
| | | TGCGTG |
| 654 | Coding Sequence of | AGCAGCGGCAACTCCAATGCCAACAGCCGGGGCCCCTCTTTCAGC |
| | Linker | TCCGGATTGGTGCCTCTGAGCCTGAGGGGCTCCCAC |
| 655 | Coding Sequence of | ATGGCAGCAATCCCCGCCCTGGACCCCGAGGCCGAGCCTAGCATG |
| | DNA Methylation | GACGTGATCCTGGTGGGCTCTAGCGAGCTGTCCTCTAGCGTGTCT |
| | Domain | CCAGGAACCGGAAGGGATCTGATCGCATACGAGGTGAAGGCCAAT |
| | | CAGCGGAACATCGAGGACATCTGTATCTGCTGTGGCAGCCTGCAG |
| | | GTGCACACACAGCACCCACTGTTCGAGGGAGGAATCTGCGCACCC |
| | | TGTAAGGATAAGTTCCTGGACGCCCTGTTTCTGTACGACGATGAC |
| | | GGCTACCAGTCCTATTGCTCTATCTGCTGTTCCGGCGAGACCCTG |
| | | CTGATCTGCGGCAATCCAGATTGTACAAGGTGCTATTGTTTTGAG |
| | | TGCGTGGACTCTCTGGTGGGACCAGGCACCAGCGGAAAGGTGCAC |
| | | GCCATGTCCAACTGGGTGTGCTACCTGTGCCTGCCATCCTCTCGC |
| | | AGCGGACTGCTGCAGCGGAGAAGGAAGTGGAGATCCCAGCTGAAG |
| | | GCCTTCTATGATAGGGAGTCTGAGAACCCCCTGGAGATGTTTGAG |
| | | ACCGTGCCAGTGTGGCGCCGGCAGCCCGTGAGGGTGCTGAGCCTG |
| | | TTCGAGGATATCAAGAAGGAGCTGACATCCCTGGGCTTTCTGGAG |
| | | TCCGGCTCTGACCCCGGACAGCTGAAGCACGTGGTGGATGTGACC |
| | | GACACAGTGCGGAAGGATGTGGAGGAGTGGGGCCCTTTCGACCTG |
| | | GTGTACGGAGCAACCCCTCCACTGGGACACACATGCGACAGACCC |
| | | CCTTCTTGGTACCTGTTCCAGTTTCACCGCCTGCTGCAGTATGCA |
| | | AGGCCAAAGCCAGGCAGCCCTAGACCATTCTTTTGGATGTTCGTG |
| | | GATAATCTGGTGCTGAACAAGGAGGATCTGGACGTGGCCAGCAGG |
| | | TTTCTGGAGATGGAGCCAGTGACCATCCCAGACGTGCACGGCGGC |
| | | TCCCTGCAGAATGCCGTGCGCGTGTGGTCTAACATCCCTGCCATC |
| | | AGAAGCAGGCACTGGGCACTGGTGAGCGAGGAGGAGCTGTCCCTG |
| | | CTGGCCCAGAATAAGCAGAGCAGCAAGCTGGCCGCCAAGTGGCCT |
| | | ACAAAGCTGGTGAAGAACTGCTTCCTGCCACTGCGGGAGTACTTC |
| | | AAGTATTTTTCCACCGAGCTGACATCTAGCCTG |
| 656 | Coding Sequence of | GGTGTTGATGGCTCCTCTGGTTCTGAAACTCCTGGTACTTCTGAG |
| | Linker | TCTGCTACTCCCGAATCT |
| 660 | Coding Sequence of | GGTGTTGATGGCTCCTCTGGTTCTGAAACTCCTGGTACTTCTGAG |
| | Linker | TCTGCTACTCCCGAATCT |
| 661 | Coding Sequence of | GGAGTCGACGGATCCAGCGGCTCCGAGACCCCAGGCACATCTGAG |
| | Linker | AGCGCCACCCCTGAGTCC |
| 662 | Coding Sequence of | GACTCCGTTGCTTTCGAGGACGTGGCCGTGAACTTCACACTTGAG |
| | Repressor | GAATGGGCCTTGCTCGACCCAAGTCAGAAGAATCTGTACAGAGAC |
| | (ZN627KRAB) | GTGATGCGGGAGACATTCAGGAATCTCGCCAGTGTCGGAAAGCAG |
| | | TGGGAAGACCAGAACATCGAAGATCCTTTCAAGATACCACGGCGC |
| | | AATATCTCCCACATTCCTGAGAGGCTGTGTGAATCTAAGGAAGGC |
| | | GGACAAGGTGAGGAA |
| 663 | Coding Sequence of | GATTACAAAGATGATGACGATAAA |
| | TAG (FLAG) | |
| 664 | linker | GGGGS |
| 665 | linker | EAAAK |
| 666 | Full Nucleic Acid | AAATCAACCTCTGGATTACAAAATTTGTGAAAGATTGACTGGTAT |
| | Sequence of 3′ UTR | TCTTAACTATGTTGCTCCTTTTACGCTATGTGGATACGCTGCTTT |
| | (WPRE) | AATGCCTTTGTATCATGCTATTGCTTCCCGTATGGCTTTCATTTT |
| | | CTCCTCCTTGTATAAATCCTGGTTGCTGTCTCTTTATGAGGAGTT |
| | | GTGGCCCGTTGTCAGGCAACGTGGCGTGGTGTGCACTGTGTTTGC |
| | | TGACGCAACCCCCACTGGTTGGGGCATTGCCACCACCTGTCAGCT |
| | | CCTTTCCGGGACTTTCGCTTTCCCCCTCCCTATTGCCACGGCGGA |
| | | ACTCATCGCCGCCTGCCTTGCCCGCTGCTGGACAGGGGCTCGGCT |
| | | GTTGGGCACTGACAATTCCGTGGTGTTGTCGGGGAAATCATCGTC |
| | | CTTTCCTTGGCTGCTCGCCTGTGTTGCCACCTGGATTCTGCGCGG |
| | | GACGTCCTTCTGCTACGTCCCTTCGGCCCTCAATCCAGCGGACCT |
| | | TCCTTCCCGCGGCCTGCTGCCGGCTCTGCGGCCTCTTCCGCGTCT |
| | | TCGCCTTCGCCCTCAGACGAGTCGGATCTCCCTTTGGGCCGCCTC |
| | | CCCGCCTGTTAATT |
| 667 | Amino Acid | MPKKKRKV |
| | Sequence of Nuclear | |
| | Localization | |
| | Sequence (SV40 | |
| | variant) | |
| 668 | Full Nucleic Acid | GACATTGATTATTGACTAGTTATTAATAGTAATCAATTACGGGGT |
| | Sequence of | CATTAGTTCATAGCCCATATATGGAGTTCCGCGTTACATAACTTA |
| | Palindrome Zinc | CGGTAAATGGCCCGCCTGGCTGACCGCCCAACGACCCCCGCCCAT |
| | Hand CLTA-PCSK9 | TGACGTCAATAATGACGTATGTTCCCATAGTAACGCCAATAGGGA |
| | | CTTTCCATTGACGTCAATGGGTGGAGTATTTACGGTAAACTGCCC |
| | | ACTTGGCAGTACATCAAGTGTATCATATGCCAAGTACGCCCCCTA |
| | | TTGACGTCAATGACGGTAAATGGCCCGCCTGGCATTATGCCCAGT |
| | | ACATGACCTTATGGGACTTTCCTACTTGGCAGTACATCTACGTAT |
| | | TAGTCATCGCTATTACCATGGTGATGCGGTTTTGGCAGTACATCA |
| | | ATGGGCGTGGATAGCGGTTTGACTCACGGGGATTTCCAAGTCTCC |
| | | ACCCCATTGACGTCAATGGGAGTTTGTTTTGGCACCAAAATCAAC |
| | | GGGACTTTCCAAAATGTCGTAACAACTCCGCCCCATTGACGCAAA |
| | | TGGGCGGTAGGCGTGTACGGTGGGAGGTCTATATAAGCAGAGCTC |
| | | TCTGGCTAACTAGAGAACCCACTGCTTACTGGCTTATCGAAATTA |
| | | ATACGACTCACTATAAGGGGGCGCTCGAGCAGGTTCAGAAGGAGA |
| | | TCAAAAACCCCCAAGGATCAAACATGCCAAAAAAGAAGAGAAAGG |
| | | TACCGAAGAAAAAAAGAAAGGTATACGACTCCGTTGCTTTCGAGG |
| | | ACGTGGCCGTGAACTTCACACTTGAGGAATGGGCCTTGCTCGACC |
| | | CAAGTCAGAAGAATCTGTACAGAGACGTGATGCGGGAGACATTCA |
| | | GGAATCTCGCCAGTGTCGGAAAGCAGTGGGAAGACCAGAACATCG |
| | | AAGATCCTTTCAAGATACCACGGCGCAATATCTCCCACATTCCTG |
| | | AGAGGCTGTGTGAATCTAAGGAAGGCGGACAAGGTGAGGAAAGCG |
| | | GAGTCGACGGATCCAGCGGCTCCGAGACCCCAGGCACATCTGAGA |
| | | GCGCCACCCCTGAGTCCGAGAGACCTTTTCAGTGCCGGATTTGTA |
| | | TGCGCAATTTCTCCCACAAGCAGCACCGCGACGCCCATATTAGAA |
| | | CACATACTGGAGAAAAGCCCTTCGCTTGCGACATCTGTGGACGGA |
| | | AGTTCGCCCGCTCCGCCAACCTGACCCGCCACACTAAAATCCATA |
| | | CCGGCAGCCAAAAGCCATTCCAATGTCGCATCTGTATGCGGAACT |
| | | TTAGCCGCTCCGACAACCTGTCCGAGCATATTCGGACTCACACAG |
| | | GGGAGAAACCATTTGCATGTGATATCTGCGGCAGAAAATTCGCTA |
| | | CCTCCGCCAACCTGTCCCGCCATACCAAGATACACACGGGATCTC |
| | | AGAAGCCCTTCCAGTGTCGAATCTGCATGCGTAACTTCAGTATCC |
| | | GCTCCACCCTGCGCGACCACATCCGCACCCACACCGGCGAGAAGC |
| | | CTTTTGCCTGTGACATTTGTGGGAGGAAATTTGCCCGCACCCCGG |
| | | TGCGCATGGGCCATACCAAGATACACCTGCGCCAAAAAGATGCGG |
| | | CCCGGGGATCTGGAGGACCCTCCTCTGGCGCCCCACCACCTAGCG |
| | | GCGGCTCCCCTGCCGGCTCTCCAACCAGCACAGAGGAGGGCACCA |
| | | GCGAGTCCGCCACACCAGAGTCTGGACCTGGCACCAGCACAGAGC |
| | | CATCCGAGGGCTCTGCCCCAGGCTCTCCTGCAGGCAGCCCTACCT |
| | | CCACCGAAGAGGGCACCAGCACAGAGCCTTCTGAGGGCAGCGCCC |
| | | CAGGCACCTCTACAGAGCCAAGCGAGAATCACGATCAGGAGTTCG |
| | | ACCCCCCTAAGGTGTACCCACCAGTGCCTGCAGAGAAGAGGAAGC |
| | | CAATCCGGGTGCTGAGCCTGTTTGATGGCATCGCCACCGGCCTGC |
| | | TGGTGCTGAAGGATCTGGGCATCCAGGTGGACCGGTACATCGCCT |
| | | CCGAGGTGTGCGAGGATTCTATCACCGTGGGCATGGTGCGCCACC |
| | | AGGGCAAGATCATGTATGTGGGCGACGTGCGGTCCGTGACACAGA |
| | | AGCACATCCAGGAGTGGGGCCCATTCGATCTGGTGATCGGCGGCA |
| | | GCCCCTGTAATGACCTGTCCATCGTGAACCCTGCAAGGAAGGGAC |
| | | TGTACGAGGGAACCGGCCGGCTGTTCTTTGAGTTTTATAGACTGC |
| | | TGCACGACGCCAGGCCTAAGGAGGGCGACGATAGACCATTCTTTT |
| | | GGCTGTTCGAGAATGTGGTGGCTATGGGCGTGAGCGATAAGAGGG |
| | | ACATCTCCAGGTTTCTGGAGTCTAACCCCGTGATGATCGATGCAA |
| | | AGGAGGTGTCCGCCGCACACAGAGCCAGGTATTTCTGGGGCAATC |
| | | TGCCAGGAATGAACAGGCCACTGGCAAGCACCGTGAATGACAAGC |
| | | TGGAGCTGCAGGAGTGCCTGGAGCACGGAAGGATCGCCAAGTTTT |
| | | CCAAGGTGCGCACAATCACCACACGGAGCAATTCCATCAAGCAGG |
| | | GCAAGGATCAGCACTTCCCCGTGTTCATGAACGAGAAGGAGGACA |
| | | TCCTGTGGTGTACCGAGATGGAGAGAGTGTTCGGCTTTCCAGTGC |
| | | ACTACACAGACGTGTCTAACATGAGCAGGCTGGCAAGGCAGCGGC |
| | | TGCTGGGCAGATCTTGGAGCGTGCCCGTGATCAGGCACCTGTTCG |
| | | CCCCTCTGAAGGAGTATTTTGCCTGCGTGAGCAGCGGCAACTCCA |
| | | ATGCCAACAGCCGGGGCCCCTCTTTCAGCTCCGGATTGGTGCCTC |
| | | TGAGCCTGAGGGGCTCCCACATGGCAGCAATCCCCGCCCTGGACC |
| | | CCGAGGCCGAGCCTAGCATGGACGTGATCCTGGTGGGCTCTAGCG |
| | | AGCTGTCCTCTAGCGTGTCTCCAGGAACCGGAAGGGATCTGATCG |
| | | CATACGAGGTGAAGGCCAATCAGCGGAACATCGAGGACATCTGTA |
| | | TCTGCTGTGGCAGCCTGCAGGTGCACACACAGCACCCACTGTTCG |
| | | AGGGAGGAATCTGCGCACCCTGTAAGGATAAGTTCCTGGACGCCC |
| | | TGTTTCTGTACGACGATGACGGCTACCAGTCCTATTGCTCTATCT |
| | | GCTGTTCCGGCGAGACCCTGCTGATCTGCGGCAATCCAGATTGTA |
| | | CAAGGTGCTATTGTTTTGAGTGCGTGGACTCTCTGGTGGGACCAG |
| | | GCACCAGCGGAAAGGTGCACGCCATGTCCAACTGGGTGTGCTACC |
| | | TGTGCCTGCCATCCTCTCGCAGCGGACTGCTGCAGCGGAGAAGGA |
| | | AGTGGAGATCCCAGCTGAAGGCCTTCTATGATAGGGAGTCTGAGA |
| | | ACCCCCTGGAGATGTTTGAGACCGTGCCAGTGTGGCGCCGGCAGC |
| | | CCGTGAGGGTGCTGAGCCTGTTCGAGGATATCAAGAAGGAGCTGA |
| | | CATCCCTGGGCTTTCTGGAGTCCGGCTCTGACCCCGGACAGCTGA |
| | | AGCACGTGGTGGATGTGACCGACACAGTGCGGAAGGATGTGGAGG |
| | | AGTGGGGCCCTTTCGACCTGGTGTACGGAGCAACCCCTCCACTGG |
| | | GACACACATGCGACAGACCCCCTTCTTGGTACCTGTTCCAGTTTC |
| | | ACCGCCTGCTGCAGTATGCAAGGCCAAAGCCAGGCAGCCCTAGAC |
| | | CATTCTTTTGGATGTTCGTGGATAATCTGGTGCTGAACAAGGAGG |
| | | ATCTGGACGTGGCCAGCAGGTTTCTGGAGATGGAGCCAGTGACCA |
| | | TCCCAGACGTGCACGGCGGCTCCCTGCAGAATGCCGTGCGCGTGT |
| | | GGTCTAACATCCCTGCCATCAGAAGCAGGCACTGGGCACTGGTGA |
| | | GCGAGGAGGAGCTGTCCCTGCTGGCCCAGAATAAGCAGAGCAGCA |
| | | AGCTGGCCGCCAAGTGGCCTACAAAGCTGGTGAAGAACTGCTTCC |
| | | TGCCACTGCGGGAGTACTTCAAGTATTTTTCCACCGAGCTGACAT |
| | | CTAGCCTGGGAGGACCCTCCTCTGGCGCCCCACCACCTAGCGGCG |
| | | GCTCCCCTGCCGGCTCTCCAACCAGCACAGAGGAGGGCACCAGCG |
| | | AGTCCGCCACACCAGAGTCTGGACCTGGCACCAGCACAGAGCCAT |
| | | CCGAGGGCTCTGCCCCAGGCTCTCCTGCAGGCAGCCCTACCTCCA |
| | | CCGAAGAGGGCACCAGCACAGAGCCTTCTGAGGGCAGCGCCCCAG |
| | | GCACCTCTACAGAGCCAAGCGAGCCCAAGAAGAAGAGGAAGGTGT |
| | | CCCGGCCAGGGGAACGGCCCTTCCAGTGTCGGATCTGCATGAGAA |
| | | ACTTTTCAACACCCTCAAAGCTGGATAGGCATACACGCACCCACA |
| | | CTGGAGAGAAACCCTTTCAGTGCAGGATATGTATGCGGAATTTTT |
| | | CCCTGGCTGAGAACCTCCGAAGGCACCTGAGAACACATACCGGGA |
| | | GTCAGAAGCCTTTCCAATGCCGGATTTGCATGAGGAACTTCTCCC |
| | | GCCAAGATAACCTGGGCCGCCACCTGCGCACACATACAGGCGAGA |
| | | AGCCATTCCAGTGTAGGATCTGCATGCGCAATTTTAGCGACGGCG |
| | | GTAACTTGGGACGCCACCTCAAAACGCATACAGGTGGCGGTGGCT |
| | | CCCAGAAGCCCTTTCAGTGCAGGATCTGCATGAGGAATTTTAGTC |
| | | AGTCCCCACATCTTAAAAGACACTTGCGGACGCATACTGGAGAGA |
| | | AGCCCTTTCAGTGTAGGATTTGTATGCGGAACTTCAGCCAGAGCA |
| | | CAAGCCTGCAACGCCATCTCAAAACTCATTTGCGCGGGTCTAGCG |
| | | GAGTCGACGGATCCAGCGGCTCCGAGACCCCAGGCACATCTGAGA |
| | | GCGCCACCCCTGAGTCCACCGGTGACTCCGTTGCTTTCGAGGACG |
| | | TGGCCGTGAACTTCACACTTGAGGAATGGGCCTTGCTCGACCCAA |
| | | GTCAGAAGAATCTGTACAGAGACGTGATGCGGGAGACATTCAGGA |
| | | ATCTCGCCAGTGTCGGAAAGCAGTGGGAAGACCAGAACATCGAAG |
| | | ATCCTTTCAAGATACCACGGCGCAATATCTCCCACATTCCTGAGA |
| | | GGCTGTGTGAATCTAAGGAAGGCGGACAAGGTGAGGAAAGCGCTG |
| | | ATTACAAAGATGATGACGATAAAGCCCCCAAGAAGAAAAGGAAGG |
| | | TCCCAAAGAAAAAAAGAAAGGTGTGAGGATCCTGAGTCTAGAAAT |
| | | CAACCTCTGGATTACAAAATTTGTGAAAGATTGACTGGTATTCTT |
| | | AACTATGTTGCTCCTTTTACGCTATGTGGATACGCTGCTTTAATG |
| | | CCTTTGTATCATGCTATTGCTTCCCGTATGGCTTTCATTTTCTCC |
| | | TCCTTGTATAAATCCTGGTTGCTGTCTCTTTATGAGGAGTTGTGG |
| | | CCCGTTGTCAGGCAACGTGGCGTGGTGTGCACTGTGTTTGCTGAC |
| | | GCAACCCCCACTGGTTGGGGCATTGCCACCACCTGTCAGCTCCTT |
| | | TCCGGGACTTTCGCTTTCCCCCTCCCTATTGCCACGGCGGAACTC |
| | | ATCGCCGCCTGCCTTGCCCGCTGCTGGACAGGGGCTCGGCTGTTG |
| | | GGCACTGACAATTCCGTGGTGTTGTCGGGGAAATCATCGTCCTTT |
| | | CCTTGGCTGCTCGCCTGTGTTGCCACCTGGATTCTGCGCGGGACG |
| | | TCCTTCTGCTACGTCCCTTCGGCCCTCAATCCAGCGGACCTTCCT |
| | | TCCCGCGGCCTGCTGCCGGCTCTGCGGCCTCTTCCGCGTCTTCGC |
| | | CTTCGCCCTCAGACGAGTCGGATCTCCCTTTGGGCCGCCTCCCCG |
| | | CCTGTTAATT |
| 669 | PCSK9 Finger 1 | TPSKLDR |
| 670 | CLTA Finger 1 | HKQHRDA |
| 671 | PCSK9 Finger 2 | LAENLRR |
| 672 | CLTA Finger 2 | RSANLTR |
| 673 | PCSK9 Finger 3 | RQDNLGR |
| 674 | CLTA Finger 3 | RSDNLSE |
| 675 | PCSK9 Finger 4 | DGGNLGR |
| 676 | CLTA Finger 4 | TSANLSR |
| 677 | PCSK9 Finger 5 | QSPHLKR |
| 678 | CLTA Finger 5 | IRSTLRD |
| 679 | PCSK9 Finger 6 | QSTSLQR |
| 680 | CLTA Finger 6 | RTPVRMG |
| 681 | Coding Sequence of | ATGCCAAAAAAGAAGAGAAAGGTACCGAAGAAAAAAAGAAAGGTA |
| | Adjacent Zinc Hand | TACAATCACGATCAGGAGTTCGACCCCCCTAAGGTGTACCCACCA |
| | CLTA-PCSK9 | GTGCCTGCAGAGAAGAGGAAGCCAATCCGGGTGCTGAGCCTGTTT |
| | | GATGGCATCGCCACCGGCCTGCTGGTGCTGAAGGATCTGGGCATC |
| | | CAGGTGGACCGGTACATCGCCTCCGAGGTGTGCGAGGATTCTATC |
| | | ACCGTGGGCATGGTGCGCCACCAGGGCAAGATCATGTATGTGGGC |
| | | GACGTGCGGTCCGTGACACAGAAGCACATCCAGGAGTGGGGCCCA |
| | | TTCGATCTGGTGATCGGCGGCAGCCCCTGTAATGACCTGTCCATC |
| | | GTGAACCCTGCAAGGAAGGGACTGTACGAGGGAACCGGCCGGCTG |
| | | TTCTTTGAGTTTTATAGACTGCTGCACGACGCCAGGCCTAAGGAG |
| | | GGCGACGATAGACCATTCTTTTGGCTGTTCGAGAATGTGGTGGCT |
| | | ATGGGCGTGAGCGATAAGAGGGACATCTCCAGGTTTCTGGAGTCT |
| | | AACCCCGTGATGATCGATGCAAAGGAGGTGTCCGCCGCACACAGA |
| | | GCCAGGTATTTCTGGGGCAATCTGCCAGGAATGAACAGGCCACTG |
| | | GCAAGCACCGTGAATGACAAGCTGGAGCTGCAGGAGTGCCTGGAG |
| | | CACGGAAGGATCGCCAAGTTTTCCAAGGTGCGCACAATCACCACA |
| | | CGGAGCAATTCCATCAAGCAGGGCAAGGATCAGCACTTCCCCGTG |
| | | TTCATGAACGAGAAGGAGGACATCCTGTGGTGTACCGAGATGGAG |
| | | AGAGTGTTCGGCTTTCCAGTGCACTACACAGACGTGTCTAACATG |
| | | AGCAGGCTGGCAAGGCAGCGGCTGCTGGGCAGATCTTGGAGCGTG |
| | | CCCGTGATCAGGCACCTGTTCGCCCCTCTGAAGGAGTATTTTGCC |
| | | TGCGTGAGCAGCGGCAACTCCAATGCCAACAGCCGGGGCCCCTCT |
| | | TTCAGCTCCGGATTGGTGCCTCTGAGCCTGAGGGGCTCCCACATG |
| | | GCAGCAATCCCCGCCCTGGACCCCGAGGCCGAGCCTAGCATGGAC |
| | | GTGATCCTGGTGGGCTCTAGCGAGCTGTCCTCTAGCGTGTCTCCA |
| | | GGAACCGGAAGGGATCTGATCGCATACGAGGTGAAGGCCAATCAG |
| | | CGGAACATCGAGGACATCTGTATCTGCTGTGGCAGCCTGCAGGTG |
| | | CACACACAGCACCCACTGTTCGAGGGAGGAATCTGCGCACCCTGT |
| | | AAGGATAAGTTCCTGGACGCCCTGTTTCTGTACGACGATGACGGC |
| | | TACCAGTCCTATTGCTCTATCTGCTGTTCCGGCGAGACCCTGCTG |
| | | ATCTGCGGCAATCCAGATTGTACAAGGTGCTATTGTTTTGAGTGC |
| | | GTGGACTCTCTGGTGGGACCAGGCACCAGCGGAAAGGTGCACGCC |
| | | ATGTCCAACTGGGTGTGCTACCTGTGCCTGCCATCCTCTCGCAGC |
| | | GGACTGCTGCAGCGGAGAAGGAAGTGGAGATCCCAGCTGAAGGCC |
| | | TTCTATGATAGGGAGTCTGAGAACCCCCTGGAGATGTTTGAGACC |
| | | GTGCCAGTGTGGCGCCGGCAGCCCGTGAGGGTGCTGAGCCTGTTC |
| | | GAGGATATCAAGAAGGAGCTGACATCCCTGGGCTTTCTGGAGTCC |
| | | GGCTCTGACCCCGGACAGCTGAAGCACGTGGTGGATGTGACCGAC |
| | | ACAGTGCGGAAGGATGTGGAGGAGTGGGGCCCTTTCGACCTGGTG |
| | | TACGGAGCAACCCCTCCACTGGGACACACATGCGACAGACCCCCT |
| | | TCTTGGTACCTGTTCCAGTTTCACCGCCTGCTGCAGTATGCAAGG |
| | | CCAAAGCCAGGCAGCCCTAGACCATTCTTTTGGATGTTCGTGGAT |
| | | AATCTGGTGCTGAACAAGGAGGATCTGGACGTGGCCAGCAGGTTT |
| | | CTGGAGATGGAGCCAGTGACCATCCCAGACGTGCACGGCGGCTCC |
| | | CTGCAGAATGCCGTGCGCGTGTGGTCTAACATCCCTGCCATCAGA |
| | | AGCAGGCACTGGGCACTGGTGAGCGAGGAGGAGCTGTCCCTGCTG |
| | | GCCCAGAATAAGCAGAGCAGCAAGCTGGCCGCCAAGTGGCCTACA |
| | | AAGCTGGTGAAGAACTGCTTCCTGCCACTGCGGGAGTACTTCAAG |
| | | TATTTTTCCACCGAGCTGACATCTAGCCTGGGAGGACCCTCCTCT |
| | | GGCGCCCCACCACCTAGCGGCGGCTCCCCTGCCGGCTCTCCAACC |
| | | AGCACAGAGGAGGGCACCAGCGAGTCCGCCACACCAGAGTCTGGA |
| | | CCTGGCACCAGCACAGAGCCATCCGAGGGCTCTGCCCCAGGCTCT |
| | | CCTGCAGGCAGCCCTACCTCCACCGAAGAGGGCACCAGCACAGAG |
| | | CCTTCTGAGGGCAGCGCCCCAGGCACCTCTACAGAGCCAAGCGAG |
| | | GAGAGACCTTTTCAGTGCCGGATTTGTATGCGCAATTTCTCCCAC |
| | | AAGCAGCACCGCGACGCCCATATTAGAACACATACTGGAGAAAAG |
| | | CCCTTCGCTTGCGACATCTGTGGACGGAAGTTCGCCCGCTCCGCC |
| | | AACCTGACCCGCCACACTAAAATCCATACCGGCAGCCAAAAGCCA |
| | | TTCCAATGTCGCATCTGTATGCGGAACTTTAGCCGCTCCGACAAC |
| | | CTGTCCGAGCATATTCGGACTCACACAGGGGAGAAACCATTTGCA |
| | | TGTGATATCTGCGGCAGAAAATTCGCTACCTCCGCCAACCTGTCC |
| | | CGCCATACCAAGATACACACGGGATCTCAGAAGCCCTTCCAGTGT |
| | | CGAATCTGCATGCGTAACTTCAGTATCCGCTCCACCCTGCGCGAC |
| | | CACATCCGCACCCACACCGGCGAGAAGCCTTTTGCCTGTGACATT |
| | | TGTGGGAGGAAATTTGCCCGCACCCCGGTGCGCATGGGCCATACC |
| | | AAGATACACCTGCGCCAAAAAGATGCGGCCCGGGGATCTAGCGGA |
| | | GTCGACGGATCCAGCGGCTCCGAGACCCCAGGCACATCTGAGAGC |
| | | GCCACCCCTGAGTCCACCGGTGACTCCGTTGCTTTCGAGGACGTG |
| | | GCCGTGAACTTCACACTTGAGGAATGGGCCTTGCTCGACCCAAGT |
| | | CAGAAGAATCTGTACAGAGACGTGATGCGGGAGACATTCAGGAAT |
| | | CTCGCCAGTGTCGGAAAGCAGTGGGAAGACCAGAACATCGAAGAT |
| | | CCTTTCAAGATACCACGGCGCAATATCTCCCACATTCCTGAGAGG |
| | | CTGTGTGAATCTAAGGAAGGCGGACAAGGTGAGGAAAGCGCTGAT |
| | | TACAAAGATGATGACGATAAAGCCCCCAAGAAGAAAAGGAAGGTC |
| | | CCAAAGAAAAAAAGAAAGGTGTGA |
| 682 | Full Nucleic Acid | GGGCGCTCGAGCAGGTTCAGAAGGAGATCAAAAACCCCCAAGGAT |
| | Sequence of | CAAACATGCCAAAAAAGAAGAGAAAGGTACCGAAGAAAAAAAGAA |
| | Adjacent Zinc Hand | AGGTATACAATCACGATCAGGAGTTCGACCCCCCTAAGGTGTACC |
| | PCSK9-CLTA | CACCAGTGCCTGCAGAGAAGAGGAAGCCAATCCGGGTGCTGAGCC |
| | | TGTTTGATGGCATCGCCACCGGCCTGCTGGTGCTGAAGGATCTGG |
| | | GCATCCAGGTGGACCGGTACATCGCCTCCGAGGTGTGCGAGGATT |
| | | CTATCACCGTGGGCATGGTGCGCCACCAGGGCAAGATCATGTATG |
| | | TGGGCGACGTGCGGTCCGTGACACAGAAGCACATCCAGGAGTGGG |
| | | GCCCATTCGATCTGGTGATCGGCGGCAGCCCCTGTAATGACCTGT |
| | | CCATCGTGAACCCTGCAAGGAAGGGACTGTACGAGGGAACCGGCC |
| | | GGCTGTTCTTTGAGTTTTATAGACTGCTGCACGACGCCAGGCCTA |
| | | AGGAGGGCGACGATAGACCATTCTTTTGGCTGTTCGAGAATGTGG |
| | | TGGCTATGGGCGTGAGCGATAAGAGGGACATCTCCAGGTTTCTGG |
| | | AGTCTAACCCCGTGATGATCGATGCAAAGGAGGTGTCCGCCGCAC |
| | | ACAGAGCCAGGTATTTCTGGGGCAATCTGCCAGGAATGAACAGGC |
| | | CACTGGCAAGCACCGTGAATGACAAGCTGGAGCTGCAGGAGTGCC |
| | | TGGAGCACGGAAGGATCGCCAAGTTTTCCAAGGTGCGCACAATCA |
| | | CCACACGGAGCAATTCCATCAAGCAGGGCAAGGATCAGCACTTCC |
| | | CCGTGTTCATGAACGAGAAGGAGGACATCCTGTGGTGTACCGAGA |
| | | TGGAGAGAGTGTTCGGCTTTCCAGTGCACTACACAGACGTGTCTA |
| | | ACATGAGCAGGCTGGCAAGGCAGCGGCTGCTGGGCAGATCTTGGA |
| | | GCGTGCCCGTGATCAGGCACCTGTTCGCCCCTCTGAAGGAGTATT |
| | | TTGCCTGCGTGAGCAGCGGCAACTCCAATGCCAACAGCCGGGGCC |
| | | CCTCTTTCAGCTCCGGATTGGTGCCTCTGAGCCTGAGGGGCTCCC |
| | | ACATGGCAGCAATCCCCGCCCTGGACCCCGAGGCCGAGCCTAGCA |
| | | TGGACGTGATCCTGGTGGGCTCTAGCGAGCTGTCCTCTAGCGTGT |
| | | CTCCAGGAACCGGAAGGGATCTGATCGCATACGAGGTGAAGGCCA |
| | | ATCAGCGGAACATCGAGGACATCTGTATCTGCTGTGGCAGCCTGC |
| | | AGGTGCACACACAGCACCCACTGTTCGAGGGAGGAATCTGCGCAC |
| | | CCTGTAAGGATAAGTTCCTGGACGCCCTGTTTCTGTACGACGATG |
| | | ACGGCTACCAGTCCTATTGCTCTATCTGCTGTTCCGGCGAGACCC |
| | | TGCTGATCTGCGGCAATCCAGATTGTACAAGGTGCTATTGTTTTG |
| | | AGTGCGTGGACTCTCTGGTGGGACCAGGCACCAGCGGAAAGGTGC |
| | | ACGCCATGTCCAACTGGGTGTGCTACCTGTGCCTGCCATCCTCTC |
| | | GCAGCGGACTGCTGCAGCGGAGAAGGAAGTGGAGATCCCAGCTGA |
| | | AGGCCTTCTATGATAGGGAGTCTGAGAACCCCCTGGAGATGTTTG |
| | | AGACCGTGCCAGTGTGGCGCCGGCAGCCCGTGAGGGTGCTGAGCC |
| | | TGTTCGAGGATATCAAGAAGGAGCTGACATCCCTGGGCTTTCTGG |
| | | AGTCCGGCTCTGACCCCGGACAGCTGAAGCACGTGGTGGATGTGA |
| | | CCGACACAGTGCGGAAGGATGTGGAGGAGTGGGGCCCTTTCGACC |
| | | TGGTGTACGGAGCAACCCCTCCACTGGGACACACATGCGACAGAC |
| | | CCCCTTCTTGGTACCTGTTCCAGTTTCACCGCCTGCTGCAGTATG |
| | | CAAGGCCAAAGCCAGGCAGCCCTAGACCATTCTTTTGGATGTTCG |
| | | TGGATAATCTGGTGCTGAACAAGGAGGATCTGGACGTGGCCAGCA |
| | | GGTTTCTGGAGATGGAGCCAGTGACCATCCCAGACGTGCACGGCG |
| | | GCTCCCTGCAGAATGCCGTGCGCGTGTGGTCTAACATCCCTGCCA |
| | | TCAGAAGCAGGCACTGGGCACTGGTGAGCGAGGAGGAGCTGTCCC |
| | | TGCTGGCCCAGAATAAGCAGAGCAGCAAGCTGGCCGCCAAGTGGC |
| | | CTACAAAGCTGGTGAAGAACTGCTTCCTGCCACTGCGGGAGTACT |
| | | TCAAGTATTTTTCCACCGAGCTGACATCTAGCCTGGGAGGACCCT |
| | | CCTCTGGCGCCCCACCACCTAGCGGCGGCTCCCCTGCCGGCTCTC |
| | | CAACCAGCACAGAGGAGGGCACCAGCGAGTCCGCCACACCAGAGT |
| | | CTGGACCTGGCACCAGCACAGAGCCATCCGAGGGCTCTGCCCCAG |
| | | GCTCTCCTGCAGGCAGCCCTACCTCCACCGAAGAGGGCACCAGCA |
| | | CAGAGCCTTCTGAGGGCAGCGCCCCAGGCACCTCTACAGAGCCAA |
| | | GCGAGTCCCGGCCAGGGGAACGGCCCTTCCAGTGTCGGATCTGCA |
| | | TGAGAAACTTTTCAACACCCTCAAAGCTGGATAGGCATACACGCA |
| | | CCCACACTGGAGAGAAACCCTTTCAGTGCAGGATATGTATGCGGA |
| | | ATTTTTCCCTGGCTGAGAACCTCCGAAGGCACCTGAGAACACATA |
| | | CCGGGAGTCAGAAGCCTTTCCAATGCCGGATTTGCATGAGGAACT |
| | | TCTCCCGCCAAGATAACCTGGGCCGCCACCTGCGCACACATACAG |
| | | GCGAGAAGCCATTCCAGTGTAGGATCTGCATGCGCAATTTTAGCG |
| | | ACGGCGGTAACTTGGGACGCCACCTCAAAACGCATACAGGTGGCG |
| | | GTGGCTCCCAGAAGCCCTTTCAGTGCAGGATCTGCATGAGGAATT |
| | | TTAGTCAGTCCCCACATCTTAAAAGACACTTGCGGACGCATACTG |
| | | GAGAGAAGCCCTTTCAGTGTAGGATTTGTATGCGGAACTTCAGCC |
| | | AGAGCACAAGCCTGCAACGCCATCTCAAAACTCATTTGCGCGGGT |
| | | CTAGCGGAGTCGACGGATCCAGCGGCTCCGAGACCCCAGGCACAT |
| | | CTGAGAGCGCCACCCCTGAGTCCACCGGTGACTCCGTTGCTTTCG |
| | | AGGACGTGGCCGTGAACTTCACACTTGAGGAATGGGCCTTGCTCG |
| | | ACCCAAGTCAGAAGAATCTGTACAGAGACGTGATGCGGGAGACAT |
| | | TCAGGAATCTCGCCAGTGTCGGAAAGCAGTGGGAAGACCAGAACA |
| | | TCGAAGATCCTTTCAAGATACCACGGCGCAATATCTCCCACATTC |
| | | CTGAGAGGCTGTGTGAATCTAAGGAAGGCGGACAAGGTGAGGAAA |
| | | GCGCTGATTACAAAGATGATGACGATAAAGCCCCCAAGAAGAAAA |
| | | GGAAGGTCCCAAAGAAAAAAAGAAAGGTGTGAGGATCCTGAGTCT |
| | | AGAAATCAACCTCTGGATTACAAAATTTGTGAAAGATTGACTGGT |
| | | ATTCTTAACTATGTTGCTCCTTTTACGCTATGTGGATACGCTGCT |
| | | TTAATGCCTTTGTATCATGCTATTGCTTCCCGTATGGCTTTCATT |
| | | TTCTCCTCCTTGTATAAATCCTGGTTGCTGTCTCTTTATGAGGAG |
| | | TTGTGGCCCGTTGTCAGGCAACGTGGCGTGGTGTGCACTGTGTTT |
| | | GCTGACGCAACCCCCACTGGTTGGGGCATTGCCACCACCTGTCAG |
| | | CTCCTTTCCGGGACTTTCGCTTTCCCCCTCCCTATTGCCACGGCG |
| | | GAACTCATCGCCGCCTGCCTTGCCCGCTGCTGGACAGGGGCTCGG |
| | | CTGTTGGGCACTGACAATTCCGTGGTGTTGTCGGGGAAATCATCG |
| | | TCCTTTCCTTGGCTGCTCGCCTGTGTTGCCACCTGGATTCTGCGC |
| | | GGGACGTCCTTCTGCTACGTCCCTTCGGCCCTCAATCCAGCGGAC |
| | | CTTCCTTCCCGCGGCCTGCTGCCGGCTCTGCGGCCTCTTCCGCGT |
| | | CTTCGCCTTCGCCCTCAGACGAGTCGGATCTCCCTTTGGGCCGCC |
| | | TCCCCGCCTGTTAATT |
| 683 | Full Nucleic Acid | GGGCGCTCGAGCAGGTTCAGAAGGAGATCAAAAACCCCCAAGGAT |
| | Sequence of | CAAACATGCCAAAAAAGAAGAGAAAGGTACCGAAGAAAAAAAGAA |
| | Adjacent Zinc Hand | AGGTATACAATCACGATCAGGAGTTCGACCCCCCTAAGGTGTACC |
| | CLTA-PCSK9 | CACCAGTGCCTGCAGAGAAGAGGAAGCCAATCCGGGTGCTGAGCC |
| | | TGTTTGATGGCATCGCCACCGGCCTGCTGGTGCTGAAGGATCTGG |
| | | GCATCCAGGTGGACCGGTACATCGCCTCCGAGGTGTGCGAGGATT |
| | | CTATCACCGTGGGCATGGTGCGCCACCAGGGCAAGATCATGTATG |
| | | TGGGCGACGTGCGGTCCGTGACACAGAAGCACATCCAGGAGTGGG |
| | | GCCCATTCGATCTGGTGATCGGCGGCAGCCCCTGTAATGACCTGT |
| | | CCATCGTGAACCCTGCAAGGAAGGGACTGTACGAGGGAACCGGCC |
| | | GGCTGTTCTTTGAGTTTTATAGACTGCTGCACGACGCCAGGCCTA |
| | | AGGAGGGCGACGATAGACCATTCTTTTGGCTGTTCGAGAATGTGG |
| | | TGGCTATGGGCGTGAGCGATAAGAGGGACATCTCCAGGTTTCTGG |
| | | AGTCTAACCCCGTGATGATCGATGCAAAGGAGGTGTCCGCCGCAC |
| | | ACAGAGCCAGGTATTTCTGGGGCAATCTGCCAGGAATGAACAGGC |
| | | CACTGGCAAGCACCGTGAATGACAAGCTGGAGCTGCAGGAGTGCC |
| | | TGGAGCACGGAAGGATCGCCAAGTTTTCCAAGGTGCGCACAATCA |
| | | CCACACGGAGCAATTCCATCAAGCAGGGCAAGGATCAGCACTTCC |
| | | CCGTGTTCATGAACGAGAAGGAGGACATCCTGTGGTGTACCGAGA |
| | | TGGAGAGAGTGTTCGGCTTTCCAGTGCACTACACAGACGTGTCTA |
| | | ACATGAGCAGGCTGGCAAGGCAGCGGCTGCTGGGCAGATCTTGGA |
| | | GCGTGCCCGTGATCAGGCACCTGTTCGCCCCTCTGAAGGAGTATT |
| | | TTGCCTGCGTGAGCAGCGGCAACTCCAATGCCAACAGCCGGGGCC |
| | | CCTCTTTCAGCTCCGGATTGGTGCCTCTGAGCCTGAGGGGCTCCC |
| | | ACATGGCAGCAATCCCCGCCCTGGACCCCGAGGCCGAGCCTAGCA |
| | | TGGACGTGATCCTGGTGGGCTCTAGCGAGCTGTCCTCTAGCGTGT |
| | | CTCCAGGAACCGGAAGGGATCTGATCGCATACGAGGTGAAGGCCA |
| | | ATCAGCGGAACATCGAGGACATCTGTATCTGCTGTGGCAGCCTGC |
| | | AGGTGCACACACAGCACCCACTGTTCGAGGGAGGAATCTGCGCAC |
| | | CCTGTAAGGATAAGTTCCTGGACGCCCTGTTTCTGTACGACGATG |
| | | ACGGCTACCAGTCCTATTGCTCTATCTGCTGTTCCGGCGAGACCC |
| | | TGCTGATCTGCGGCAATCCAGATTGTACAAGGTGCTATTGTTTTG |
| | | AGTGCGTGGACTCTCTGGTGGGACCAGGCACCAGCGGAAAGGTGC |
| | | ACGCCATGTCCAACTGGGTGTGCTACCTGTGCCTGCCATCCTCTC |
| | | GCAGCGGACTGCTGCAGCGGAGAAGGAAGTGGAGATCCCAGCTGA |
| | | AGGCCTTCTATGATAGGGAGTCTGAGAACCCCCTGGAGATGTTTG |
| | | AGACCGTGCCAGTGTGGCGCCGGCAGCCCGTGAGGGTGCTGAGCC |
| | | TGTTCGAGGATATCAAGAAGGAGCTGACATCCCTGGGCTTTCTGG |
| | | AGTCCGGCTCTGACCCCGGACAGCTGAAGCACGTGGTGGATGTGA |
| | | CCGACACAGTGCGGAAGGATGTGGAGGAGTGGGGCCCTTTCGACC |
| | | TGGTGTACGGAGCAACCCCTCCACTGGGACACACATGCGACAGAC |
| | | CCCCTTCTTGGTACCTGTTCCAGTTTCACCGCCTGCTGCAGTATG |
| | | CAAGGCCAAAGCCAGGCAGCCCTAGACCATTCTTTTGGATGTTCG |
| | | TGGATAATCTGGTGCTGAACAAGGAGGATCTGGACGTGGCCAGCA |
| | | GGTTTCTGGAGATGGAGCCAGTGACCATCCCAGACGTGCACGGCG |
| | | GCTCCCTGCAGAATGCCGTGCGCGTGTGGTCTAACATCCCTGCCA |
| | | TCAGAAGCAGGCACTGGGCACTGGTGAGCGAGGAGGAGCTGTCCC |
| | | TGCTGGCCCAGAATAAGCAGAGCAGCAAGCTGGCCGCCAAGTGGC |
| | | CTACAAAGCTGGTGAAGAACTGCTTCCTGCCACTGCGGGAGTACT |
| | | TCAAGTATTTTTCCACCGAGCTGACATCTAGCCTGGGAGGACCCT |
| | | CCTCTGGCGCCCCACCACCTAGCGGCGGCTCCCCTGCCGGCTCTC |
| | | CAACCAGCACAGAGGAGGGCACCAGCGAGTCCGCCACACCAGAGT |
| | | CTGGACCTGGCACCAGCACAGAGCCATCCGAGGGCTCTGCCCCAG |
| | | GCTCTCCTGCAGGCAGCCCTACCTCCACCGAAGAGGGCACCAGCA |
| | | CAGAGCCTTCTGAGGGCAGCGCCCCAGGCACCTCTACAGAGCCAA |
| | | GCGAGGAGAGACCTTTTCAGTGCCGGATTTGTATGCGCAATTTCT |
| | | CCCACAAGCAGCACCGCGACGCCCATATTAGAACACATACTGGAG |
| | | AAAAGCCCTTCGCTTGCGACATCTGTGGACGGAAGTTCGCCCGCT |
| | | CCGCCAACCTGACCCGCCACACTAAAATCCATACCGGCAGCCAAA |
| | | AGCCATTCCAATGTCGCATCTGTATGCGGAACTTTAGCCGCTCCG |
| | | ACAACCTGTCCGAGCATATTCGGACTCACACAGGGGAGAAACCAT |
| | | TTGCATGTGATATCTGCGGCAGAAAATTCGCTACCTCCGCCAACC |
| | | TGTCCCGCCATACCAAGATACACACGGGATCTCAGAAGCCCTTCC |
| | | AGTGTCGAATCTGCATGCGTAACTTCAGTATCCGCTCCACCCTGC |
| | | GCGACCACATCCGCACCCACACCGGCGAGAAGCCTTTTGCCTGTG |
| | | ACATTTGTGGGAGGAAATTTGCCCGCACCCCGGTGCGCATGGGCC |
| | | ATACCAAGATACACCTGCGCCAAAAAGATGCGGCCCGGGGATCTA |
| | | GCGGAGTCGACGGATCCAGCGGCTCCGAGACCCCAGGCACATCTG |
| | | AGAGCGCCACCCCTGAGTCCACCGGTGACTCCGTTGCTTTCGAGG |
| | | ACGTGGCCGTGAACTTCACACTTGAGGAATGGGCCTTGCTCGACC |
| | | CAAGTCAGAAGAATCTGTACAGAGACGTGATGCGGGAGACATTCA |
| | | GGAATCTCGCCAGTGTCGGAAAGCAGTGGGAAGACCAGAACATCG |
| | | AAGATCCTTTCAAGATACCACGGCGCAATATCTCCCACATTCCTG |
| | | AGAGGCTGTGTGAATCTAAGGAAGGCGGACAAGGTGAGGAAAGCG |
| | | CTGATTACAAAGATGATGACGATAAAGCCCCCAAGAAGAAAAGGA |
| | | AGGTCCCAAAGAAAAAAAGAAAGGTGTGAGGATCCTGAGTCTAGA |
| | | AATCAACCTCTGGATTACAAAATTTGTGAAAGATTGACTGGTATT |
| | | CTTAACTATGTTGCTCCTTTTACGCTATGTGGATACGCTGCTTTA |
| | | ATGCCTTTGTATCATGCTATTGCTTCCCGTATGGCTTTCATTTTC |
| | | TCCTCCTTGTATAAATCCTGGTTGCTGTCTCTTTATGAGGAGTTG |
| | | TGGCCCGTTGTCAGGCAACGTGGCGTGGTGTGCACTGTGTTTGCT |
| | | GACGCAACCCCCACTGGTTGGGGCATTGCCACCACCTGTCAGCTC |
| | | CTTTCCGGGACTTTCGCTTTCCCCCTCCCTATTGCCACGGCGGAA |
| | | CTCATCGCCGCCTGCCTTGCCCGCTGCTGGACAGGGGCTCGGCTG |
| | | TTGGGCACTGACAATTCCGTGGTGTTGTCGGGGAAATCATCGTCC |
| | | TTTCCTTGGCTGCTCGCCTGTGTTGCCACCTGGATTCTGCGCGGG |
| | | ACGTCCTTCTGCTACGTCCCTTCGGCCCTCAATCCAGCGGACCTT |
| | | CCTTCCCGCGGCCTGCTGCCGGCTCTGCGGCCTCTTCCGCGTCTT |
| | | CGCCTTCGCCCTCAGACGAGTCGGATCTCCCTTTGGGCCGCCTCC |
| | | CCGCCTGTTAATT |
| 684 | Amino Acid | LEDKKYSIGLAIGTNSVGWAVITDEYKVPSKKFKVLGNTDRHSIK |
| | Sequence of DNA | KNLIGALLFDSGETAEATRLKRTARRRYTRRKNRICYLQEIFSNE |
| | Binding Domain | MAKVDDSFFHRLEESFLVEEDKKHERHPIFGNIVDEVAYHEKYPT |
| | | IYHLRKKLVDSTDKADLRLIYLALAHMIKFRGHFLIEGDLNPDNS |
| | | DVDKLFIQLVQTYNQLFEENPINASGVDAKAILSARLSKSRRLEN |
| | | LIAQLPGEKKNGLFGNLIALSLGLTPNFKSNFDLAEDAKLQLSKD |
| | | TYDDDLDNLLAQIGDQYADLFLAAKNLSDAILLSDILRVNTEITK |
| | | APLSASMIKRYDEHHQDLILLKALVRQQLPEKYKEIFFDQSKNGY |
| | | AGYIDGGASQEEFYKFIKPILEKMDGTEELLVKLNREDLLRKQRT |
| | | FDNGSIPHQIHLGELHAILRRQEDFYPFLKDNREKIEKILTFRIP |
| | | YYVGPLARGNSRFAWMTRKSEETITPWNFEEVVDKGASAQSFIER |
| | | MTNFDKNLPNEKVLPKHSLLYEYFTVYNELTKVKYVTEGMRKPAF |
| | | LSGEQKKAIVDLLFKTNRKVTVKQLKEDYFKKIECFDSVEISGVE |
| | | DRFNASLGTYHDLLKIIKDKDFLDNEENEDILEDIVLTLTLFEDR |
| | | EMIEERLKTYAHLFDDKVMKQLKRRRYTGWGRLSRKLINGIRDKQ |
| | | SGKTILDFLKSDGFANRNFMQLIHDDSLTFKEDIQKAQVSGQGDS |
| | | LHEHIANLAGSPAIKKGILQTVKVVDELVKVMGRHKPENIVIEMA |
| | | RENQTTQKGQKNSRERMKRIEEGIKELGSQILKEHPVENTQLQNE |
| | | KLYLYYLQNGRDMYVDQELDINRLSDYDVDAIVPQSFLKDDSIDN |
| | | KVLTRSDKNRGKSDNVPSEEVVKKMKNYWRQLLNAKLITQRKFDN |
| | | LTKAERGGLSELDKAGFIKRQLVETRQITKHVAQILDSRMNTKYD |
| | | ENDKLIREVKVITLKSKLVSDFRKDFQFYKVREINNYHHAHDAYL |
| | | NAVVGTALIKKYPKLESEFVYGDYKVYDVRKMIAKSEQEIGKATA |
| | | KYFFYSNIMNFFKTEITLANGEIRKRPLIEINGETGEIVWDKGRD |
| | | FATVRKVLSMPQVNIVKKTEVQTGGFSKESILPKRNSDKLIARKK |
| | | DWDPKKYGGFDSPTVAYSVLVVAKVEKGKSKKLKSVKELLGITIM |
| | | ERSSFEKNPIDFLEAKGYKEVKKDLIIKLPKYSLFELENGRKRML |
| | | ASAGELQKGNELALPSKYVNFLYLASHYEKLKGSPEDNEQKQLFV |
| | | EQHKHYLDEIIEQISEFSKRVILADANLDKVLSAYNKHRDKPIRE |
| | | QAENIIHLFTLTNLGAPAAFKYFDTTIDRKRYTSTKEVLDATLIH |
| | | QSITGLYETRIDLSQLGGD |
| 685 | Amino Acid | RTLVTFKDVFVDFTREEWKLLDTAQQIVYRNVMLENYKNLVSLGY |
| | Sequence of | QLTKPDVILRLEKGEEPWLV |
| | Repressor | |
| 686 | Coding Sequence of | CCGAAGAAAAAGCGCAAGGTC |
| | Nuclear Localization | |
| | Signal | |
| 687 | Coding Sequence of | AATCACGATCAGGAGTTCGACCCCCCTAAGGTGTACCCACCAGTG |
| | DNA Methylation | CCTGCAGAGAAGAGGAAGCCAATCCGGGTGCTGAGCCTGTTTGAT |
| | Domain | GGCATCGCCACCGGCCTGCTGGTGCTGAAGGATCTGGGCATCCAG |
| | | GTGGACCGGTACATCGCCTCCGAGGTGTGCGAGGATTCTATCACC |
| | | GTGGGCATGGTGCGCCACCAGGGCAAGATCATGTATGTGGGCGAC |
| | | GTGCGGTCCGTGACACAGAAGCACATCCAGGAGTGGGGCCCATTC |
| | | GATCTGGTGATCGGCGGCAGCCCCTGTAATGACCTGTCCATCGTG |
| | | AACCCTGCAAGGAAGGGACTGTACGAGGGAACCGGCCGGCTGTTC |
| | | TTTGAGTTTTATAGACTGCTGCACGACGCCAGGCCTAAGGAGGGC |
| | | GACGATAGACCATTCTTTTGGCTGTTCGAGAATGTGGTGGCTATG |
| | | GGCGTGAGCGATAAGAGGGACATCTCCAGGTTTCTGGAGTCTAAC |
| | | CCCGTGATGATCGATGCAAAGGAGGTGTCCGCCGCACACAGAGCC |
| | | AGGTATTTCTGGGGCAATCTGCCAGGAATGAACAGGCCACTGGCA |
| | | AGCACCGTGAATGACAAGCTGGAGCTGCAGGAGTGCCTGGAGCAC |
| | | GGAAGGATCGCCAAGTTTTCCAAGGTGCGCACAATCACCACACGG |
| | | AGCAATTCCATCAAGCAGGGCAAGGATCAGCACTTCCCCGTGTTC |
| | | ATGAACGAGAAGGAGGACATCCTGTGGTGTACCGAGATGGAGAGA |
| | | GTGTTCGGCTTTCCAGTGCACTACACAGACGTGTCTAACATGAGC |
| | | AGGCTGGCAAGGCAGCGGCTGCTGGGCAGATCTTGGAGCGTGCCC |
| | | GTGATCAGGCACCTGTTCGCCCCTCTGAAGGAGTATTTTGCCTGC |
| | | GTG |
| 688 | Coding Sequence of | CTCGAGGACAAGAAGTACAGCATCGGCCTGGCCATCGGCACCAAC |
| | DNA Binding | TCTGTGGGCTGGGCCGTGATCACCGACGAGTACAAGGTGCCCAGC |
| | Domain | AAGAAATTCAAGGTGCTGGGCAACACCGACCGGCACAGCATCAAG |
| | | AAGAACCTGATCGGAGCCCTGCTGTTCGACAGCGGCGAAACAGCC |
| | | GAGGCCACCCGGCTGAAGAGAACCGCCAGAAGAAGATACACCAGA |
| | | CGGAAGAACCGGATCTGCTATCTGCAAGAGATCTTCAGCAACGAG |
| | | ATGGCCAAGGTGGACGACAGCTTCTTCCACAGACTGGAAGAGTCC |
| | | TTCCTGGTGGAAGAGGATAAGAAGCACGAGCGGCACCCCATCTTC |
| | | GGCAACATCGTGGACGAGGTGGCCTACCACGAGAAGTACCCCACC |
| | | ATCTACCACCTGAGAAAGAAACTGGTGGACAGCACCGACAAGGCC |
| | | GACCTGCGGCTGATCTATCTGGCCCTGGCCCACATGATCAAGTTC |
| | | CGGGGCCACTTCCTGATCGAGGGCGACCTGAACCCCGACAACAGC |
| | | GACGTGGACAAGCTGTTCATCCAGCTGGTGCAGACCTACAACCAG |
| | | CTGTTCGAGGAAAACCCCATCAACGCCAGCGGCGTGGACGCCAAG |
| | | GCCATCCTGTCTGCCAGACTGAGCAAGAGCAGACGGCTGGAAAAT |
| | | CTGATCGCCCAGCTGCCCGGCGAGAAGAAGAATGGCCTGTTCGGC |
| | | AACCTGATTGCCCTGAGCCTGGGCCTGACCCCCAACTTCAAGAGC |
| | | AACTTCGACCTGGCCGAGGATGCCAAACTGCAGCTGAGCAAGGAC |
| | | ACCTACGACGACGACCTGGACAACCTGCTGGCCCAGATCGGCGAC |
| | | CAGTACGCCGACCTGTTTCTGGCCGCCAAGAACCTGTCCGACGCC |
| | | ATCCTGCTGAGCGACATCCTGAGAGTGAACACCGAGATCACCAAG |
| | | GCCCCCCTGAGCGCCTCTATGATCAAGAGATACGACGAGCACCAC |
| | | CAGGACCTGACCCTGCTGAAAGCTCTCGTGCGGCAGCAGCTGCCT |
| | | GAGAAGTACAAAGAGATTTTCTTCGACCAGAGCAAGAACGGCTAC |
| | | GCCGGCTACATTGACGGCGGAGCCAGCCAGGAAGAGTTCTACAAG |
| | | TTCATCAAGCCCATCCTGGAAAAGATGGACGGCACCGAGGAACTG |
| | | CTCGTGAAGCTGAACAGAGAGGACCTGCTGCGGAAGCAGCGGACC |
| | | TTCGACAACGGCAGCATCCCCCACCAGATCCACCTGGGAGAGCTG |
| | | CACGCCATTCTGCGGCGGCAGGAAGATTTTTACCCATTCCTGAAG |
| | | GACAACCGGGAAAAGATCGAGAAGATCCTGACCTTCCGCATCCCC |
| | | TACTACGTGGGCCCTCTGGCCAGGGGAAACAGCAGATTCGCCTGG |
| | | ATGACCAGAAAGAGCGAGGAAACCATCACCCCCTGGAACTTCGAG |
| | | GAAGTGGTGGACAAGGGCGCTTCCGCCCAGAGCTTCATCGAGCGG |
| | | ATGACCAACTTCGATAAGAACCTGCCCAACGAGAAGGTGCTGCCC |
| | | AAGCACAGCCTGCTGTACGAGTACTTCACCGTGTATAACGAGCTG |
| | | ACCAAAGTGAAATACGTGACCGAGGGAATGAGAAAGCCCGCCTTC |
| | | CTGAGCGGCGAGCAGAAAAAGGCCATCGTGGACCTGCTGTTCAAG |
| | | ACCAACCGGAAAGTGACCGTGAAGCAGCTGAAAGAGGACTACTTC |
| | | AAGAAAATCGAGTGCTTCGACTCCGTGGAAATCTCCGGCGTGGAA |
| | | GATCGGTTCAACGCCTCCCTGGGCACATACCACGATCTGCTGAAA |
| | | ATTATCAAGGACAAGGACTTCCTGGACAATGAGGAAAACGAGGAC |
| | | ATTCTGGAAGATATCGTGCTGACCCTGACACTGTTTGAGGACAGA |
| | | GAGATGATCGAGGAACGGCTGAAAACCTATGCCCACCTGTTCGAC |
| | | GACAAAGTGATGAAGCAGCTGAAGCGGCGGAGATACACCGGCTGG |
| | | GGCAGGCTGAGCCGGAAGCTGATCAACGGCATCCGGGACAAGCAG |
| | | TCCGGCAAGACAATCCTGGATTTCCTGAAGTCCGACGGCTTCGCC |
| | | AACAGAAACTTCATGCAGCTGATCCACGACGACAGCCTGACCTTT |
| | | AAAGAGGACATCCAGAAAGCCCAGGTGTCCGGCCAGGGCGATAGC |
| | | CTGCACGAGCACATTGCCAATCTGGCCGGCAGCCCCGCCATTAAG |
| | | AAGGGCATCCTGCAGACAGTGAAGGTGGTGGACGAGCTCGTGAAA |
| | | GTGATGGGCCGGCACAAGCCCGAGAACATCGTGATCGAAATGGCC |
| | | AGAGAGAACCAGACCACCCAGAAGGGACAGAAGAACAGCCGCGAG |
| | | AGAATGAAGCGGATCGAAGAGGGCATCAAAGAGCTGGGCAGCCAG |
| | | ATCCTGAAAGAACACCCCGTGGAAAACACCCAGCTGCAGAACGAG |
| | | AAGCTGTACCTGTACTACCTGCAGAATGGGCGGGATATGTACGTG |
| | | GACCAGGAACTGGACATCAACCGGCTGTCCGACTACGATGTGGAC |
| | | GCCATCGTGCCTCAGAGCTTTCTGAAGGACGACTCCATCGACAAC |
| | | AAGGTGCTGACCAGAAGCGACAAGAACCGGGGCAAGAGCGACAAC |
| | | GTGCCCTCCGAAGAGGTCGTGAAGAAGATGAAGAACTACTGGCGG |
| | | CAGCTGCTGAACGCCAAGCTGATTACCCAGAGAAAGTTCGACAAT |
| | | CTGACCAAGGCCGAGAGAGGCGGCCTGAGCGAACTGGATAAGGCC |
| | | GGCTTCATCAAGAGACAGCTGGTGGAAACCCGGCAGATCACAAAG |
| | | CACGTGGCACAGATCCTGGACTCCCGGATGAACACTAAGTACGAC |
| | | GAGAATGACAAGCTGATCCGGGAAGTGAAAGTGATCACCCTGAAG |
| | | TCCAAGCTGGTGTCCGATTTCCGGAAGGATTTCCAGTTTTACAAA |
| | | GTGCGCGAGATCAACAACTACCACCACGCCCACGACGCCTACCTG |
| | | AACGCCGTCGTGGGAACCGCCCTGATCAAAAAGTACCCTAAGCTG |
| | | GAAAGCGAGTTCGTGTACGGCGACTACAAGGTGTACGACGTGCGG |
| | | AAGATGATCGCCAAGAGCGAGCAGGAAATCGGCAAGGCTACCGCC |
| | | AAGTACTTCTTCTACAGCAACATCATGAACTTTTTCAAGACCGAG |
| | | ATTACCCTGGCCAACGGCGAGATCCGGAAGCGGCCTCTGATCGAG |
| | | ACAAACGGCGAAACCGGGGAGATCGTGTGGGATAAGGGCCGGGAT |
| | | TTTGCCACCGTGCGGAAAGTGCTGAGCATGCCCCAAGTGAATATC |
| | | GTGAAAAAGACCGAGGTGCAGACAGGCGGCTTCAGCAAAGAGTCT |
| | | ATCCTGCCCAAGAGGAACAGCGATAAGCTGATCGCCAGAAAGAAG |
| | | GACTGGGACCCTAAGAAGTACGGCGGCTTCGACAGCCCCACCGTG |
| | | GCCTATTCTGTGCTGGTGGTGGCCAAAGTGGAAAAGGGCAAGTCC |
| | | AAGAAACTGAAGAGTGTGAAAGAGCTGCTGGGGATCACCATCATG |
| | | GAAAGAAGCAGCTTCGAGAAGAATCCCATCGACTTTCTGGAAGCC |
| | | AAGGGCTACAAAGAAGTGAAAAAGGACCTGATCATCAAGCTGCCT |
| | | AAGTACTCCCTGTTCGAGCTGGAAAACGGCCGGAAGAGAATGCTG |
| | | GCCTCTGCCGGCGAACTGCAGAAGGGAAACGAACTGGCCCTGCCC |
| | | TCCAAATATGTGAACTTCCTGTACCTGGCCAGCCACTATGAGAAG |
| | | CTGAAGGGCTCCCCCGAGGATAATGAGCAGAAACAGCTGTTTGTG |
| | | GAACAGCACAAGCACTACCTGGACGAGATCATCGAGCAGATCAGC |
| | | GAGTTCTCCAAGAGAGTGATCCTGGCCGACGCTAATCTGGACAAA |
| | | GTGCTGTCCGCCTACAACAAGCACCGGGATAAGCCCATCAGAGAG |
| | | CAGGCCGAGAATATCATCCACCTGTTTACCCTGACCAATCTGGGA |
| | | GCCCCTGCCGCCTTCAAGTACTTTGACACCACCATCGACCGGAAG |
| | | AGGTACACCAGCACCAAAGAGGTGCTGGACGCCACCCTGATCCAC |
| | | CAGAGCATCACCGGCCTGTACGAGACACGGATCGACCTGTCTCAG |
| | | CTGGGAGGCGAC |
| 689 | Coding Sequence of | CGGACCCTGGTGACATTCAAGGACGTGTTCGTGGACTTCACCCGG |
| | Repressor | GAGGAGTGGAAGCTGCTGGACACAGCCCAGCAGATCGTGTACAGG |
| | | AACGTGATGCTGGAGAACTATAAGAATCTGGTGTCTCTGGGCTAC |
| | | CAGCTGACAAAGCCAGATGTGATCCTGCGGCTGGAGAAGGGAGAG |
| | | GAGCCCTGGCTGGTG |
| 690 | Amino Acid | DKKYSIGLAIGTNSVGWAVITDEYKVPSKKFKVLGNTDRHSIKKN |
| | Sequence of DNA | LIGALLFDSGETAEATRLKRTARRRYTRRKNRICYLQEIFSNEMA |
| | Binding Domain | KVDDSFFHRLEESFLVEEDKKHERHPIFGNIVDEVAYHEKYPTIY |
| | | HLRKKLVDSTDKADLRLIYLALAHMIKFRGHFLIEGDLNPDNSDV |
| | | DKLFIQLVQTYNQLFEENPINASGVDAKAILSARLSKSRRLENLI |
| | | AQLPGEKKNGLFGNLIALSLGLTPNFKSNFDLAEDAKLQLSKDTY |
| | | DDDLDNLLAQIGDQYADLFLAAKNLSDAILLSDILRVNTEITKAP |
| | | LSASMIKRYDEHHQDLILLKALVRQQLPEKYKEIFFDQSKNGYAG |
| | | YIDGGASQEEFYKFIKPILEKMDGTEELLVKLNREDLLRKQRTED |
| | | NGSIPHQIHLGELHAILRRQEDFYPFLKDNREKIEKILTFRIPYY |
| | | VGPLARGNSRFAWMTRKSEETITPWNFEEVVDKGASAQSFIERMT |
| | | NFDKNLPNEKVLPKHSLLYEYFTVYNELTKVKYVTEGMRKPAFLS |
| | | GEQKKAIVDLLFKTNRKVTVKQLKEDYFKKIECFDSVEISGVEDR |
| | | FNASLGTYHDLLKIIKDKDFLDNEENEDILEDIVLTLTLFEDREM |
| | | IEERLKTYAHLFDDKVMKQLKRRRYTGWGRLSRKLINGIRDKQSG |
| | | KTILDFLKSDGFANRNFMQLIHDDSLTFKEDIQKAQVSGQGDSLH |
| | | EHIANLAGSPAIKKGILQTVKVVDELVKVMGRHKPENIVIEMARE |
| | | NQTTQKGQKNSRERMKRIEEGIKELGSQILKEHPVENTQLQNEKL |
| | | YLYYLQNGRDMYVDQELDINRLSDYDVDAIVPQSFLKDDSIDNKV |
| | | LTRSDKNRGKSDNVPSEEVVKKMKNYWRQLLNAKLITQRKFDNLT |
| | | KAERGGLSELDKAGFIKRQLVETRQITKHVAQILDSRMNTKYDEN |
| | | DKLIREVKVITLKSKLVSDFRKDFQFYKVREINNYHHAHDAYLNA |
| | | VVGTALIKKYPKLESEFVYGDYKVYDVRKMIAKSEQEIGKATAKY |
| | | FFYSNIMNFFKTEITLANGEIRKRPLIEINGETGEIVWDKGRDFA |
| | | TVRKVLSMPQVNIVKKTEVQTGGFSKESILPKRNSDKLIARKKDW |
| | | DPKKYGGFDSPTVAYSVLVVAKVEKGKSKKLKSVKELLGITIMER |
| | | SSFEKNPIDFLEAKGYKEVKKDLIIKLPKYSLFELENGRKRMLAS |
| | | AGELQKGNELALPSKYVNFLYLASHYEKLKGSPEDNEQKQLFVEQ |
| | | HKHYLDEIIEQISEFSKRVILADANLDKVLSAYNKHRDKPIREQA |
| | | ENIIHLFTLTNLGAPAAFKYFDTTIDRKRYTSTKEVLDATLIHQS |
| | | ITGLYETRIDLSQLGGD |
| 691 | Coding Sequence of | GACAAGAAGTACAGCATCGGCCTGGCCATCGGCACCAACTCTGTG |
| | DNA Binding | GGCTGGGCCGTGATCACCGACGAGTACAAGGTGCCCAGCAAGAAA |
| | Domain | TTCAAGGTGCTGGGCAACACCGACCGGCACAGCATCAAGAAGAAC |
| | | CTGATCGGCGCCCTGCTGTTCGACAGCGGAGAAACAGCCGAGGCC |
| | | ACCCGGCTGAAGAGAACCGCCAGAAGAAGATACACCAGACGGAAG |
| | | AACCGGATCTGCTATCTGCAAGAGATCTTCAGCAACGAGATGGCC |
| | | AAGGTGGACGACAGCTTCTTCCACAGACTGGAAGAGTCCTTCCTG |
| | | GTGGAAGAGGATAAGAAGCACGAGCGGCACCCCATCTTCGGCAAC |
| | | ATCGTGGACGAGGTGGCCTACCACGAGAAGTACCCCACCATCTAC |
| | | CACCTGAGAAAGAAACTGGTGGACAGCACCGACAAGGCCGACCTG |
| | | CGGCTGATCTATCTGGCCCTGGCCCACATGATCAAGTTCCGGGGC |
| | | CACTTCCTGATCGAGGGCGACCTGAACCCCGACAACAGCGACGTG |
| | | GACAAGCTGTTCATCCAGCTGGTGCAGACCTACAACCAGCTGTTC |
| | | GAGGAAAACCCCATCAACGCCAGCGGCGTGGACGCCAAGGCCATC |
| | | CTGTCTGCCAGACTGAGCAAGAGCAGACGGCTGGAAAATCTGATC |
| | | GCCCAGCTGCCCGGCGAGAAGAAGAATGGCCTGTTCGGCAACCTG |
| | | ATTGCCCTGAGCCTGGGCCTGACCCCCAACTTCAAGAGCAACTTC |
| | | GACCTGGCCGAGGATGCCAAACTGCAGCTGAGCAAGGACACCTAC |
| | | GACGACGACCTGGACAACCTGCTGGCCCAGATCGGCGACCAGTAC |
| | | GCCGACCTGTTTCTGGCCGCCAAGAACCTGTCCGACGCCATCCTG |
| | | CTGAGCGACATCCTGAGAGTGAACACCGAGATCACCAAGGCCCCC |
| | | CTGAGCGCCTCTATGATCAAGAGATACGACGAGCACCACCAGGAC |
| | | CTGACCCTGCTGAAAGCTCTCGTGCGGCAGCAGCTGCCTGAGAAG |
| | | TACAAAGAGATTTTCTTCGACCAGAGCAAGAACGGCTACGCCGGC |
| | | TACATCGATGGCGGAGCCAGCCAGGAAGAGTTCTACAAGTTCATC |
| | | AAGCCCATCCTGGAAAAGATGGACGGCACCGAGGAACTGCTCGTG |
| | | AAGCTGAACAGAGAGGACCTGCTGCGGAAGCAGCGGACCTTCGAC |
| | | AACGGCAGCATCCCCCACCAGATCCACCTGGGAGAGCTGCACGCC |
| | | ATTCTGCGGCGGCAGGAAGATTTTTACCCATTCCTGAAGGACAAC |
| | | CGGGAAAAGATCGAGAAGATCCTGACCTTCCGCATCCCCTACTAC |
| | | GTGGGCCCTCTGGCCAGGGGAAACAGCAGATTCGCCTGGATGACC |
| | | AGAAAGAGCGAGGAAACCATCACCCCCTGGAACTTCGAGGAAGTG |
| | | GTGGACAAGGGCGCCAGCGCCCAGAGCTTCATCGAGCGGATGACC |
| | | AACTTCGATAAGAACCTGCCCAACGAGAAGGTGCTGCCCAAGCAC |
| | | AGCCTGCTGTACGAGTACTTCACCGTGTACAACGAGCTGACCAAA |
| | | GTGAAATACGTGACCGAGGGAATGAGAAAGCCCGCCTTCCTGAGC |
| | | GGCGAGCAGAAAAAAGCCATCGTGGACCTGCTGTTCAAGACCAAC |
| | | CGGAAAGTGACCGTGAAGCAGCTGAAAGAGGACTACTTCAAGAAA |
| | | ATCGAGTGCTTCGACTCCGTGGAAATCTCCGGCGTGGAAGATCGG |
| | | TTCAACGCCTCCCTGGGCACATACCACGATCTGCTGAAAATTATC |
| | | AAGGACAAGGACTTCCTGGACAATGAGGAAAACGAGGACATTCTG |
| | | GAAGATATCGTGCTGACCCTGACACTGTTTGAGGACAGAGAGATG |
| | | ATCGAGGAACGGCTGAAAACCTATGCCCACCTGTTCGACGACAAA |
| | | GTGATGAAGCAGCTGAAGCGGCGGAGATACACCGGCTGGGGCAGG |
| | | CTGAGCCGGAAGCTGATCAACGGCATCCGGGACAAGCAGTCCGGC |
| | | AAGACAATCCTGGATTTCCTGAAGTCCGACGGCTTCGCCAACAGA |
| | | AACTTCATGCAGCTGATCCACGACGACAGCCTGACCTTTAAAGAG |
| | | GACATCCAGAAAGCCCAGGTGTCCGGCCAGGGCGATAGCCTGCAC |
| | | GAGCACATTGCCAATCTGGCCGGCAGCCCCGCCATTAAGAAGGGC |
| | | ATCCTGCAGACAGTGAAGGTGGTGGACGAGCTCGTGAAAGTGATG |
| | | GGCCGGCACAAGCCCGAGAACATCGTGATCGAAATGGCCAGAGAG |
| | | AACCAGACCACCCAGAAGGGACAGAAGAACAGCCGCGAGAGAATG |
| | | AAGCGGATCGAAGAGGGCATCAAAGAGCTGGGCAGCCAGATCCTG |
| | | AAAGAACACCCCGTGGAAAACACCCAGCTGCAGAACGAGAAGCTG |
| | | TACCTGTACTACCTGCAGAATGGGCGGGATATGTACGTGGACCAG |
| | | GAACTGGACATCAACCGGCTGTCCGACTACGATGTGGACGCTATC |
| | | GTGCCTCAGAGCTTTCTGAAGGACGACTCCATCGATAACAAAGTG |
| | | CTGACTCGGAGCGACAAGAACCGGGGCAAGAGCGACAACGTGCCC |
| | | TCCGAAGAGGTCGTGAAGAAGATGAAGAACTACTGGCGCCAGCTG |
| | | CTGAATGCCAAGCTGATTACCCAGAGGAAGTTCGACAATCTGACC |
| | | AAGGCCGAGAGAGGCGGCCTGAGCGAACTGGATAAGGCCGGCTTC |
| | | ATCAAGAGACAGCTGGTGGAAACCCGGCAGATCACAAAGCACGTG |
| | | GCACAGATCCTGGACTCCCGGATGAACACTAAGTACGACGAGAAC |
| | | GACAAACTGATCCGGGAAGTGAAAGTGATCACCCTGAAGTCCAAG |
| | | CTGGTGTCCGATTTCCGGAAGGATTTCCAGTTTTACAAAGTGCGC |
| | | GAGATCAACAACTACCACCACGCCCACGACGCCTACCTGAACGCC |
| | | GTCGTGGGAACCGCCCTGATCAAAAAGTACCCTAAGCTGGAAAGC |
| | | GAGTTCGTGTACGGCGACTACAAGGTGTACGACGTGCGGAAGATG |
| | | ATCGCCAAGAGCGAGCAGGAAATCGGCAAGGCTACCGCCAAGTAC |
| | | TTCTTCTACAGCAACATCATGAACTTTTTCAAGACCGAGATTACC |
| | | CTGGCCAACGGCGAGATCCGGAAGCGGCCTCTGATCGAGACAAAC |
| | | GGCGAAACAGGCGAGATCGTGTGGGATAAGGGCCGGGACTTTGCC |
| | | ACCGTGCGGAAAGTGCTGTCTATGCCCCAAGTGAATATCGTGAAA |
| | | AAGACCGAGGTGCAGACAGGCGGCTTCAGCAAAGAGTCTATCCTG |
| | | CCCAAGAGGAACAGCGACAAGCTGATCGCCAGAAAGAAGGACTGG |
| | | GACCCTAAGAAGTACGGCGGCTTCGACAGCCCCACCGTGGCCTAT |
| | | TCTGTGCTGGTGGTGGCCAAAGTGGAAAAGGGCAAGTCCAAGAAA |
| | | CTGAAGAGTGTGAAAGAGCTGCTGGGGATCACCATCATGGAAAGA |
| | | AGCAGCTTCGAGAAGAATCCCATCGACTTTCTGGAAGCCAAGGGC |
| | | TACAAAGAAGTGAAAAAGGACCTGATCATCAAGCTGCCTAAGTAC |
| | | TCCCTGTTCGAGCTGGAAAACGGCCGGAAGAGAATGCTGGCCTCT |
| | | GCCGGCGAACTGCAGAAGGGAAACGAACTGGCCCTGCCCTCCAAA |
| | | TATGTGAACTTCCTGTACCTGGCCAGCCACTATGAGAAGCTGAAG |
| | | GGCTCCCCCGAGGATAATGAGCAGAAACAGCTGTTTGTGGAACAG |
| | | CACAAACACTACCTGGACGAGATCATCGAGCAGATCAGCGAGTTC |
| | | TCCAAGAGAGTGATCCTGGCCGACGCTAATCTGGACAAGGTGCTG |
| | | AGCGCCTACAACAAGCACAGAGACAAGCCTATCAGAGAGCAGGCC |
| | | GAGAATATCATCCACCTGTTTACCCTGACCAATCTGGGAGCCCCT |
| | | GCCGCCTTCAAGTACTTTGACACCACCATCGACCGGAAGAGGTAC |
| | | ACCAGCACCAAAGAGGTGCTGGACGCCACCCTGATCCACCAGAGC |
| | | ATCACCGGCCTGTACGAGACACGGATCGACCTGTCTCAGCTGGGA |
| | | GGCGAC |
| 692 | Coding Sequence of | AGCGGAAGTGAGACCCCAGGTACATCCGAATCAGCAACGCCTGAA |
| | Linker | AGC |
| 693 | Coding Sequence of | CGGACACTGGTGACCTTCAAGGATGTATTTGTGGACTTCACCAGG |
| | Repressor | GAGGAGTGGAAGCTGCTGGACACTGCTCAGCAGATCGTGTACAGA |
| | | AATGTGATGCTGGAGAACTATAAGAACCTGGTTTCCTTGGGTTAT |
| | | CAGCTTACTAAGCCAGATGTGATCCTCCGGTTGGAGAAGGGAGAA |
| | | GAGCCC |
| 694 | Full Nucleic Acid | GACATTGATTATTGACTAGTTATTAATAGTAATCAATTACGGGGT |
| | Sequence of | CATTAGTTCATAGCCCATATATGGAGTTCCGCGTTACATAACTTA |
| | CRISPRoff | CGGTAAATGGCCCGCCTGGCTGACCGCCCAACGACCCCCGCCCAT |
| | | TGACGTCAATAATGACGTATGTTCCCATAGTAACGCCAATAGGGA |
| | | CTTTCCATTGACGTCAATGGGTGGAGTATTTACGGTAAACTGCCC |
| | | ACTTGGCAGTACATCAAGTGTATCATATGCCAAGTACGCCCCCTA |
| | | TTGACGTCAATGACGGTAAATGGCCCGCCTGGCATTATGCCCAGT |
| | | ACATGACCTTATGGGACTTTCCTACTTGGCAGTACATCTACGTAT |
| | | TAGTCATCGCTATTACCATGGTGATGCGGTTTTGGCAGTACATCA |
| | | ATGGGCGTGGATAGCGGTTTGACTCACGGGGATTTCCAAGTCTCC |
| | | ACCCCATTGACGTCAATGGGAGTTTGTTTTGGCACCAAAATCAAC |
| | | GGGACTTTCCAAAATGTCGTAACAACTCCGCCCCATTGACGCAAA |
| | | TGGGCGGTAGGCGTGTACGGTGGGAGGTCTATATAAGCAGAGCTC |
| | | TCTGGCTAACTAGAGAACCCACTGCTTACTGGCTTATCGAAATTA |
| | | ATACGACTCACTATAAGGAGACCCAAGCTACCGGTGCCACCATGT |
| | | ACCCATACGATGTTCCAGATTACGCTTCGCCGAAGAAAAAGCGCA |
| | | AGGTCAATCACGATCAGGAGTTCGACCCCCCTAAGGTGTACCCAC |
| | | CAGTGCCTGCAGAGAAGAGGAAGCCAATCCGGGTGCTGAGCCTGT |
| | | TTGATGGCATCGCCACCGGCCTGCTGGTGCTGAAGGATCTGGGCA |
| | | TCCAGGTGGACCGGTACATCGCCTCCGAGGTGTGCGAGGATTCTA |
| | | TCACCGTGGGCATGGTGCGCCACCAGGGCAAGATCATGTATGTGG |
| | | GCGACGTGCGGTCCGTGACACAGAAGCACATCCAGGAGTGGGGCC |
| | | CATTCGATCTGGTGATCGGCGGCAGCCCCTGTAATGACCTGTCCA |
| | | TCGTGAACCCTGCAAGGAAGGGACTGTACGAGGGAACCGGCCGGC |
| | | TGTTCTTTGAGTTTTATAGACTGCTGCACGACGCCAGGCCTAAGG |
| | | AGGGCGACGATAGACCATTCTTTTGGCTGTTCGAGAATGTGGTGG |
| | | CTATGGGCGTGAGCGATAAGAGGGACATCTCCAGGTTTCTGGAGT |
| | | CTAACCCCGTGATGATCGATGCAAAGGAGGTGTCCGCCGCACACA |
| | | GAGCCAGGTATTTCTGGGGCAATCTGCCAGGAATGAACAGGCCAC |
| | | TGGCAAGCACCGTGAATGACAAGCTGGAGCTGCAGGAGTGCCTGG |
| | | AGCACGGAAGGATCGCCAAGTTTTCCAAGGTGCGCACAATCACCA |
| | | CACGGAGCAATTCCATCAAGCAGGGCAAGGATCAGCACTTCCCCG |
| | | TGTTCATGAACGAGAAGGAGGACATCCTGTGGTGTACCGAGATGG |
| | | AGAGAGTGTTCGGCTTTCCAGTGCACTACACAGACGTGTCTAACA |
| | | TGAGCAGGCTGGCAAGGCAGCGGCTGCTGGGCAGATCTTGGAGCG |
| | | TGCCCGTGATCAGGCACCTGTTCGCCCCTCTGAAGGAGTATTTTG |
| | | CCTGCGTGAGCAGCGGCAACTCCAATGCCAACAGCCGGGGCCCCT |
| | | CTTTCAGCTCCGGATTGGTGCCTCTGAGCCTGAGGGGCTCCCACA |
| | | TGGCAGCAATCCCCGCCCTGGACCCCGAGGCCGAGCCTAGCATGG |
| | | ACGTGATCCTGGTGGGCTCTAGCGAGCTGTCCTCTAGCGTGTCTC |
| | | CAGGAACCGGAAGGGATCTGATCGCATACGAGGTGAAGGCCAATC |
| | | AGCGGAACATCGAGGACATCTGTATCTGCTGTGGCAGCCTGCAGG |
| | | TGCACACACAGCACCCACTGTTCGAGGGAGGAATCTGCGCACCCT |
| | | GTAAGGATAAGTTCCTGGACGCCCTGTTTCTGTACGACGATGACG |
| | | GCTACCAGTCCTATTGCTCTATCTGCTGTTCCGGCGAGACCCTGC |
| | | TGATCTGCGGCAATCCAGATTGTACAAGGTGCTATTGTTTTGAGT |
| | | GCGTGGACTCTCTGGTGGGACCAGGCACCAGCGGAAAGGTGCACG |
| | | CCATGTCCAACTGGGTGTGCTACCTGTGCCTGCCATCCTCTCGCA |
| | | GCGGACTGCTGCAGCGGAGAAGGAAGTGGAGATCCCAGCTGAAGG |
| | | CCTTCTATGATAGGGAGTCTGAGAACCCCCTGGAGATGTTTGAGA |
| | | CCGTGCCAGTGTGGCGCCGGCAGCCCGTGAGGGTGCTGAGCCTGT |
| | | TCGAGGATATCAAGAAGGAGCTGACATCCCTGGGCTTTCTGGAGT |
| | | CCGGCTCTGACCCCGGACAGCTGAAGCACGTGGTGGATGTGACCG |
| | | ACACAGTGCGGAAGGATGTGGAGGAGTGGGGCCCTTTCGACCTGG |
| | | TGTACGGAGCAACCCCTCCACTGGGACACACATGCGACAGACCCC |
| | | CTTCTTGGTACCTGTTCCAGTTTCACCGCCTGCTGCAGTATGCAA |
| | | GGCCAAAGCCAGGCAGCCCTAGACCATTCTTTTGGATGTTCGTGG |
| | | ATAATCTGGTGCTGAACAAGGAGGATCTGGACGTGGCCAGCAGGT |
| | | TTCTGGAGATGGAGCCAGTGACCATCCCAGACGTGCACGGCGGCT |
| | | CCCTGCAGAATGCCGTGCGCGTGTGGTCTAACATCCCTGCCATCA |
| | | GAAGCAGGCACTGGGCACTGGTGAGCGAGGAGGAGCTGTCCCTGC |
| | | TGGCCCAGAATAAGCAGAGCAGCAAGCTGGCCGCCAAGTGGCCTA |
| | | CAAAGCTGGTGAAGAACTGCTTCCTGCCACTGCGGGAGTACTTCA |
| | | AGTATTTTTCCACCGAGCTGACATCTAGCCTGGGAGGACCCTCCT |
| | | CTGGCGCCCCACCACCTAGCGGCGGCTCCCCTGCCGGCTCTCCAA |
| | | CCAGCACAGAGGAGGGCACCAGCGAGTCCGCCACACCAGAGTCTG |
| | | GACCTGGCACCAGCACAGAGCCATCCGAGGGCTCTGCCCCAGGCT |
| | | CTCCTGCAGGCAGCCCTACCTCCACCGAAGAGGGCACCAGCACAG |
| | | AGCCTTCTGAGGGCAGCGCCCCAGGCACCTCTACAGAGCCAAGCG |
| | | AGCTCGAGGACAAGAAGTACAGCATCGGCCTGGCCATCGGCACCA |
| | | ACTCTGTGGGCTGGGCCGTGATCACCGACGAGTACAAGGTGCCCA |
| | | GCAAGAAATTCAAGGTGCTGGGCAACACCGACCGGCACAGCATCA |
| | | AGAAGAACCTGATCGGAGCCCTGCTGTTCGACAGCGGCGAAACAG |
| | | CCGAGGCCACCCGGCTGAAGAGAACCGCCAGAAGAAGATACACCA |
| | | GACGGAAGAACCGGATCTGCTATCTGCAAGAGATCTTCAGCAACG |
| | | AGATGGCCAAGGTGGACGACAGCTTCTTCCACAGACTGGAAGAGT |
| | | CCTTCCTGGTGGAAGAGGATAAGAAGCACGAGCGGCACCCCATCT |
| | | TCGGCAACATCGTGGACGAGGTGGCCTACCACGAGAAGTACCCCA |
| | | CCATCTACCACCTGAGAAAGAAACTGGTGGACAGCACCGACAAGG |
| | | CCGACCTGCGGCTGATCTATCTGGCCCTGGCCCACATGATCAAGT |
| | | TCCGGGGCCACTTCCTGATCGAGGGCGACCTGAACCCCGACAACA |
| | | GCGACGTGGACAAGCTGTTCATCCAGCTGGTGCAGACCTACAACC |
| | | AGCTGTTCGAGGAAAACCCCATCAACGCCAGCGGCGTGGACGCCA |
| | | AGGCCATCCTGTCTGCCAGACTGAGCAAGAGCAGACGGCTGGAAA |
| | | ATCTGATCGCCCAGCTGCCCGGCGAGAAGAAGAATGGCCTGTTCG |
| | | GCAACCTGATTGCCCTGAGCCTGGGCCTGACCCCCAACTTCAAGA |
| | | GCAACTTCGACCTGGCCGAGGATGCCAAACTGCAGCTGAGCAAGG |
| | | ACACCTACGACGACGACCTGGACAACCTGCTGGCCCAGATCGGCG |
| | | ACCAGTACGCCGACCTGTTTCTGGCCGCCAAGAACCTGTCCGACG |
| | | CCATCCTGCTGAGCGACATCCTGAGAGTGAACACCGAGATCACCA |
| | | AGGCCCCCCTGAGCGCCTCTATGATCAAGAGATACGACGAGCACC |
| | | ACCAGGACCTGACCCTGCTGAAAGCTCTCGTGCGGCAGCAGCTGC |
| | | CTGAGAAGTACAAAGAGATTTTCTTCGACCAGAGCAAGAACGGCT |
| | | ACGCCGGCTACATTGACGGCGGAGCCAGCCAGGAAGAGTTCTACA |
| | | AGTTCATCAAGCCCATCCTGGAAAAGATGGACGGCACCGAGGAAC |
| | | TGCTCGTGAAGCTGAACAGAGAGGACCTGCTGCGGAAGCAGCGGA |
| | | CCTTCGACAACGGCAGCATCCCCCACCAGATCCACCTGGGAGAGC |
| | | TGCACGCCATTCTGCGGCGGCAGGAAGATTTTTACCCATTCCTGA |
| | | AGGACAACCGGGAAAAGATCGAGAAGATCCTGACCTTCCGCATCC |
| | | CCTACTACGTGGGCCCTCTGGCCAGGGGAAACAGCAGATTCGCCT |
| | | GGATGACCAGAAAGAGCGAGGAAACCATCACCCCCTGGAACTTCG |
| | | AGGAAGTGGTGGACAAGGGCGCTTCCGCCCAGAGCTTCATCGAGC |
| | | GGATGACCAACTTCGATAAGAACCTGCCCAACGAGAAGGTGCTGC |
| | | CCAAGCACAGCCTGCTGTACGAGTACTTCACCGTGTATAACGAGC |
| | | TGACCAAAGTGAAATACGTGACCGAGGGAATGAGAAAGCCCGCCT |
| | | TCCTGAGCGGCGAGCAGAAAAAGGCCATCGTGGACCTGCTGTTCA |
| | | AGACCAACCGGAAAGTGACCGTGAAGCAGCTGAAAGAGGACTACT |
| | | TCAAGAAAATCGAGTGCTTCGACTCCGTGGAAATCTCCGGCGTGG |
| | | AAGATCGGTTCAACGCCTCCCTGGGCACATACCACGATCTGCTGA |
| | | AAATTATCAAGGACAAGGACTTCCTGGACAATGAGGAAAACGAGG |
| | | ACATTCTGGAAGATATCGTGCTGACCCTGACACTGTTTGAGGACA |
| | | GAGAGATGATCGAGGAACGGCTGAAAACCTATGCCCACCTGTTCG |
| | | ACGACAAAGTGATGAAGCAGCTGAAGCGGCGGAGATACACCGGCT |
| | | GGGGCAGGCTGAGCCGGAAGCTGATCAACGGCATCCGGGACAAGC |
| | | AGTCCGGCAAGACAATCCTGGATTTCCTGAAGTCCGACGGCTTCG |
| | | CCAACAGAAACTTCATGCAGCTGATCCACGACGACAGCCTGACCT |
| | | TTAAAGAGGACATCCAGAAAGCCCAGGTGTCCGGCCAGGGCGATA |
| | | GCCTGCACGAGCACATTGCCAATCTGGCCGGCAGCCCCGCCATTA |
| | | AGAAGGGCATCCTGCAGACAGTGAAGGTGGTGGACGAGCTCGTGA |
| | | AAGTGATGGGCCGGCACAAGCCCGAGAACATCGTGATCGAAATGG |
| | | CCAGAGAGAACCAGACCACCCAGAAGGGACAGAAGAACAGCCGCG |
| | | AGAGAATGAAGCGGATCGAAGAGGGCATCAAAGAGCTGGGCAGCC |
| | | AGATCCTGAAAGAACACCCCGTGGAAAACACCCAGCTGCAGAACG |
| | | AGAAGCTGTACCTGTACTACCTGCAGAATGGGCGGGATATGTACG |
| | | TGGACCAGGAACTGGACATCAACCGGCTGTCCGACTACGATGTGG |
| | | ACGCCATCGTGCCTCAGAGCTTTCTGAAGGACGACTCCATCGACA |
| | | ACAAGGTGCTGACCAGAAGCGACAAGAACCGGGGCAAGAGCGACA |
| | | ACGTGCCCTCCGAAGAGGTCGTGAAGAAGATGAAGAACTACTGGC |
| | | GGCAGCTGCTGAACGCCAAGCTGATTACCCAGAGAAAGTTCGACA |
| | | ATCTGACCAAGGCCGAGAGAGGCGGCCTGAGCGAACTGGATAAGG |
| | | CCGGCTTCATCAAGAGACAGCTGGTGGAAACCCGGCAGATCACAA |
| | | AGCACGTGGCACAGATCCTGGACTCCCGGATGAACACTAAGTACG |
| | | ACGAGAATGACAAGCTGATCCGGGAAGTGAAAGTGATCACCCTGA |
| | | AGTCCAAGCTGGTGTCCGATTTCCGGAAGGATTTCCAGTTTTACA |
| | | AAGTGCGCGAGATCAACAACTACCACCACGCCCACGACGCCTACC |
| | | TGAACGCCGTCGTGGGAACCGCCCTGATCAAAAAGTACCCTAAGC |
| | | TGGAAAGCGAGTTCGTGTACGGCGACTACAAGGTGTACGACGTGC |
| | | GGAAGATGATCGCCAAGAGCGAGCAGGAAATCGGCAAGGCTACCG |
| | | CCAAGTACTTCTTCTACAGCAACATCATGAACTTTTTCAAGACCG |
| | | AGATTACCCTGGCCAACGGCGAGATCCGGAAGCGGCCTCTGATCG |
| | | AGACAAACGGCGAAACCGGGGAGATCGTGTGGGATAAGGGCCGGG |
| | | ATTTTGCCACCGTGCGGAAAGTGCTGAGCATGCCCCAAGTGAATA |
| | | TCGTGAAAAAGACCGAGGTGCAGACAGGCGGCTTCAGCAAAGAGT |
| | | CTATCCTGCCCAAGAGGAACAGCGATAAGCTGATCGCCAGAAAGA |
| | | AGGACTGGGACCCTAAGAAGTACGGCGGCTTCGACAGCCCCACCG |
| | | TGGCCTATTCTGTGCTGGTGGTGGCCAAAGTGGAAAAGGGCAAGT |
| | | CCAAGAAACTGAAGAGTGTGAAAGAGCTGCTGGGGATCACCATCA |
| | | TGGAAAGAAGCAGCTTCGAGAAGAATCCCATCGACTTTCTGGAAG |
| | | CCAAGGGCTACAAAGAAGTGAAAAAGGACCTGATCATCAAGCTGC |
| | | CTAAGTACTCCCTGTTCGAGCTGGAAAACGGCCGGAAGAGAATGC |
| | | TGGCCTCTGCCGGCGAACTGCAGAAGGGAAACGAACTGGCCCTGC |
| | | CCTCCAAATATGTGAACTTCCTGTACCTGGCCAGCCACTATGAGA |
| | | AGCTGAAGGGCTCCCCCGAGGATAATGAGCAGAAACAGCTGTTTG |
| | | TGGAACAGCACAAGCACTACCTGGACGAGATCATCGAGCAGATCA |
| | | GCGAGTTCTCCAAGAGAGTGATCCTGGCCGACGCTAATCTGGACA |
| | | AAGTGCTGTCCGCCTACAACAAGCACCGGGATAAGCCCATCAGAG |
| | | AGCAGGCCGAGAATATCATCCACCTGTTTACCCTGACCAATCTGG |
| | | GAGCCCCTGCCGCCTTCAAGTACTTTGACACCACCATCGACCGGA |
| | | AGAGGTACACCAGCACCAAAGAGGTGCTGGACGCCACCCTGATCC |
| | | ACCAGAGCATCACCGGCCTGTACGAGACACGGATCGACCTGTCTC |
| | | AGCTGGGAGGCGACAGCCCCAAGAAGAAGAGAAAGGTGGGAGTCG |
| | | ACGGATCCAGCGGCTCCGAGACCCCAGGCACATCTGAGAGCGCCA |
| | | CCCCTGAGTCCCGGACCCTGGTGACATTCAAGGACGTGTTCGTGG |
| | | ACTTCACCCGGGAGGAGTGGAAGCTGCTGGACACAGCCCAGCAGA |
| | | TCGTGTACAGGAACGTGATGCTGGAGAACTATAAGAATCTGGTGT |
| | | CTCTGGGCTACCAGCTGACAAAGCCAGATGTGATCCTGCGGCTGG |
| | | AGAAGGGAGAGGAGCCCTGGCTGGTGTAGTCTAGAAATCAACCTC |
| | | TGGATTACAAAATTTGTGAAAGATTGACTGGTATTCTTAACTATG |
| | | TTGCTCCTTTTACGCTATGTGGATACGCTGCTTTAATGCCTTTGT |
| | | ATCATGCTATTGCTTCCCGTATGGCTTTCATTTTCTCCTCCTTGT |
| | | ATAAATCCTGGTTGCTGTCTCTTTATGAGGAGTTGTGGCCCGTTG |
| | | TCAGGCAACGTGGCGTGGTGTGCACTGTGTTTGCTGACGCAACCC |
| | | CCACTGGTTGGGGCATTGCCACCACCTGTCAGCTCCTTTCCGGGA |
| | | CTTTCGCTTTCCCCCTCCCTATTGCCACGGCGGAACTCATCGCCG |
| | | CCTGCCTTGCCCGCTGCTGGACAGGGGCTCGGCTGTTGGGCACTG |
| | | ACAATTCCGTGGTGTTGTCGGGGAAATCATCGTCCTTTCCTTGGC |
| | | TGCTCGCCTGTGTTGCCACCTGGATTCTGCGCGGGACGTCCTTCT |
| | | GCTACGTCCCTTCGGCCCTCAATCCAGCGGACCTTCCTTCCCGCG |
| | | GCCTGCTGCCGGCTCTGCGGCCTCTTCCGCGTCTTCGCCTTCGCC |
| | | CTCAGACGAGTCGGATCTCCCTTTGGGCCGCCTCCCCGCCTGTTA |
| | | ATT |
| 695 | Full Nucleic Acid | AGACCCAAGCTACCGGTGCCACCATG |
| | Sequence of 5′UTR | |
| 696 | Full Nucleic Acid | GACATTGATTATTGACTAGTTATTAATAGTAATCAATTACGGGGT |
| | Sequence of | CATTAGTTCATAGCCCATATATGGAGTTCCGCGTTACATAACTTA |
| | CRISPRi Construct | CGGTAAATGGCCCGCCTGGCTGACCGCCCAACGACCCCCGCCCAT |
| | | TGACGTCAATAATGACGTATGTTCCCATAGTAACGCCAATAGGGA |
| | | CTTTCCATTGACGTCAATGGGTGGAGTATTTACGGTAAACTGCCC |
| | | ACTTGGCAGTACATCAAGTGTATCATATGCCAAGTACGCCCCCTA |
| | | TTGACGTCAATGACGGTAAATGGCCCGCCTGGCATTATGCCCAGT |
| | | ACATGACCTTATGGGACTTTCCTACTTGGCAGTACATCTACGTAT |
| | | TAGTCATCGCTATTACCATGGTGATGCGGTTTTGGCAGTACATCA |
| | | ATGGGCGTGGATAGCGGTTTGACTCACGGGGATTTCCAAGTCTCC |
| | | ACCCCATTGACGTCAATGGGAGTTTGTTTTGGCACCAAAATCAAC |
| | | GGGACTTTCCAAAATGTCGTAACAACTCCGCCCCATTGACGCAAA |
| | | TGGGCGGTAGGCGTGTACGGTGGGAGGTCTATATAAGCAGAGCTC |
| | | TCTGGCTAACTAGAGAACCCACTGCTTACTGGCTTATCGAAATTA |
| | | ATACGACTCACTATAAGGAGACCCAAGCTGGCTAGCCGCCACCAT |
| | | GGGTACCATGCCAAAAAAGAAGAGAAAGGTACCGAAGAAAAAAAG |
| | | AAAGGTATACGACAAGAAGTACAGCATCGGCCTGGCCATCGGCAC |
| | | CAACTCTGTGGGCTGGGCCGTGATCACCGACGAGTACAAGGTGCC |
| | | CAGCAAGAAATTCAAGGTGCTGGGCAACACCGACCGGCACAGCAT |
| | | CAAGAAGAACCTGATCGGCGCCCTGCTGTTCGACAGCGGAGAAAC |
| | | AGCCGAGGCCACCCGGCTGAAGAGAACCGCCAGAAGAAGATACAC |
| | | CAGACGGAAGAACCGGATCTGCTATCTGCAAGAGATCTTCAGCAA |
| | | CGAGATGGCCAAGGTGGACGACAGCTTCTTCCACAGACTGGAAGA |
| | | GTCCTTCCTGGTGGAAGAGGATAAGAAGCACGAGCGGCACCCCAT |
| | | CTTCGGCAACATCGTGGACGAGGTGGCCTACCACGAGAAGTACCC |
| | | CACCATCTACCACCTGAGAAAGAAACTGGTGGACAGCACCGACAA |
| | | GGCCGACCTGCGGCTGATCTATCTGGCCCTGGCCCACATGATCAA |
| | | GTTCCGGGGCCACTTCCTGATCGAGGGCGACCTGAACCCCGACAA |
| | | CAGCGACGTGGACAAGCTGTTCATCCAGCTGGTGCAGACCTACAA |
| | | CCAGCTGTTCGAGGAAAACCCCATCAACGCCAGCGGCGTGGACGC |
| | | CAAGGCCATCCTGTCTGCCAGACTGAGCAAGAGCAGACGGCTGGA |
| | | AAATCTGATCGCCCAGCTGCCCGGCGAGAAGAAGAATGGCCTGTT |
| | | CGGCAACCTGATTGCCCTGAGCCTGGGCCTGACCCCCAACTTCAA |
| | | GAGCAACTTCGACCTGGCCGAGGATGCCAAACTGCAGCTGAGCAA |
| | | GGACACCTACGACGACGACCTGGACAACCTGCTGGCCCAGATCGG |
| | | CGACCAGTACGCCGACCTGTTTCTGGCCGCCAAGAACCTGTCCGA |
| | | CGCCATCCTGCTGAGCGACATCCTGAGAGTGAACACCGAGATCAC |
| | | CAAGGCCCCCCTGAGCGCCTCTATGATCAAGAGATACGACGAGCA |
| | | CCACCAGGACCTGACCCTGCTGAAAGCTCTCGTGCGGCAGCAGCT |
| | | GCCTGAGAAGTACAAAGAGATTTTCTTCGACCAGAGCAAGAACGG |
| | | CTACGCCGGCTACATCGATGGCGGAGCCAGCCAGGAAGAGTTCTA |
| | | CAAGTTCATCAAGCCCATCCTGGAAAAGATGGACGGCACCGAGGA |
| | | ACTGCTCGTGAAGCTGAACAGAGAGGACCTGCTGCGGAAGCAGCG |
| | | GACCTTCGACAACGGCAGCATCCCCCACCAGATCCACCTGGGAGA |
| | | GCTGCACGCCATTCTGCGGCGGCAGGAAGATTTTTACCCATTCCT |
| | | GAAGGACAACCGGGAAAAGATCGAGAAGATCCTGACCTTCCGCAT |
| | | CCCCTACTACGTGGGCCCTCTGGCCAGGGGAAACAGCAGATTCGC |
| | | CTGGATGACCAGAAAGAGCGAGGAAACCATCACCCCCTGGAACTT |
| | | CGAGGAAGTGGTGGACAAGGGCGCCAGCGCCCAGAGCTTCATCGA |
| | | GCGGATGACCAACTTCGATAAGAACCTGCCCAACGAGAAGGTGCT |
| | | GCCCAAGCACAGCCTGCTGTACGAGTACTTCACCGTGTACAACGA |
| | | GCTGACCAAAGTGAAATACGTGACCGAGGGAATGAGAAAGCCCGC |
| | | CTTCCTGAGCGGCGAGCAGAAAAAAGCCATCGTGGACCTGCTGTT |
| | | CAAGACCAACCGGAAAGTGACCGTGAAGCAGCTGAAAGAGGACTA |
| | | CTTCAAGAAAATCGAGTGCTTCGACTCCGTGGAAATCTCCGGCGT |
| | | GGAAGATCGGTTCAACGCCTCCCTGGGCACATACCACGATCTGCT |
| | | GAAAATTATCAAGGACAAGGACTTCCTGGACAATGAGGAAAACGA |
| | | GGACATTCTGGAAGATATCGTGCTGACCCTGACACTGTTTGAGGA |
| | | CAGAGAGATGATCGAGGAACGGCTGAAAACCTATGCCCACCTGTT |
| | | CGACGACAAAGTGATGAAGCAGCTGAAGCGGCGGAGATACACCGG |
| | | CTGGGGCAGGCTGAGCCGGAAGCTGATCAACGGCATCCGGGACAA |
| | | GCAGTCCGGCAAGACAATCCTGGATTTCCTGAAGTCCGACGGCTT |
| | | CGCCAACAGAAACTTCATGCAGCTGATCCACGACGACAGCCTGAC |
| | | CTTTAAAGAGGACATCCAGAAAGCCCAGGTGTCCGGCCAGGGCGA |
| | | TAGCCTGCACGAGCACATTGCCAATCTGGCCGGCAGCCCCGCCAT |
| | | TAAGAAGGGCATCCTGCAGACAGTGAAGGTGGTGGACGAGCTCGT |
| | | GAAAGTGATGGGCCGGCACAAGCCCGAGAACATCGTGATCGAAAT |
| | | GGCCAGAGAGAACCAGACCACCCAGAAGGGACAGAAGAACAGCCG |
| | | CGAGAGAATGAAGCGGATCGAAGAGGGCATCAAAGAGCTGGGCAG |
| | | CCAGATCCTGAAAGAACACCCCGTGGAAAACACCCAGCTGCAGAA |
| | | CGAGAAGCTGTACCTGTACTACCTGCAGAATGGGCGGGATATGTA |
| | | CGTGGACCAGGAACTGGACATCAACCGGCTGTCCGACTACGATGT |
| | | GGACGCTATCGTGCCTCAGAGCTTTCTGAAGGACGACTCCATCGA |
| | | TAACAAAGTGCTGACTCGGAGCGACAAGAACCGGGGCAAGAGCGA |
| | | CAACGTGCCCTCCGAAGAGGTCGTGAAGAAGATGAAGAACTACTG |
| | | GCGCCAGCTGCTGAATGCCAAGCTGATTACCCAGAGGAAGTTCGA |
| | | CAATCTGACCAAGGCCGAGAGAGGCGGCCTGAGCGAACTGGATAA |
| | | GGCCGGCTTCATCAAGAGACAGCTGGTGGAAACCCGGCAGATCAC |
| | | AAAGCACGTGGCACAGATCCTGGACTCCCGGATGAACACTAAGTA |
| | | CGACGAGAACGACAAACTGATCCGGGAAGTGAAAGTGATCACCCT |
| | | GAAGTCCAAGCTGGTGTCCGATTTCCGGAAGGATTTCCAGTTTTA |
| | | CAAAGTGCGCGAGATCAACAACTACCACCACGCCCACGACGCCTA |
| | | CCTGAACGCCGTCGTGGGAACCGCCCTGATCAAAAAGTACCCTAA |
| | | GCTGGAAAGCGAGTTCGTGTACGGCGACTACAAGGTGTACGACGT |
| | | GCGGAAGATGATCGCCAAGAGCGAGCAGGAAATCGGCAAGGCTAC |
| | | CGCCAAGTACTTCTTCTACAGCAACATCATGAACTTTTTCAAGAC |
| | | CGAGATTACCCTGGCCAACGGCGAGATCCGGAAGCGGCCTCTGAT |
| | | CGAGACAAACGGCGAAACAGGCGAGATCGTGTGGGATAAGGGCCG |
| | | GGACTTTGCCACCGTGCGGAAAGTGCTGTCTATGCCCCAAGTGAA |
| | | TATCGTGAAAAAGACCGAGGTGCAGACAGGCGGCTTCAGCAAAGA |
| | | GTCTATCCTGCCCAAGAGGAACAGCGACAAGCTGATCGCCAGAAA |
| | | GAAGGACTGGGACCCTAAGAAGTACGGCGGCTTCGACAGCCCCAC |
| | | CGTGGCCTATTCTGTGCTGGTGGTGGCCAAAGTGGAAAAGGGCAA |
| | | GTCCAAGAAACTGAAGAGTGTGAAAGAGCTGCTGGGGATCACCAT |
| | | CATGGAAAGAAGCAGCTTCGAGAAGAATCCCATCGACTTTCTGGA |
| | | AGCCAAGGGCTACAAAGAAGTGAAAAAGGACCTGATCATCAAGCT |
| | | GCCTAAGTACTCCCTGTTCGAGCTGGAAAACGGCCGGAAGAGAAT |
| | | GCTGGCCTCTGCCGGCGAACTGCAGAAGGGAAACGAACTGGCCCT |
| | | GCCCTCCAAATATGTGAACTTCCTGTACCTGGCCAGCCACTATGA |
| | | GAAGCTGAAGGGCTCCCCCGAGGATAATGAGCAGAAACAGCTGTT |
| | | TGTGGAACAGCACAAACACTACCTGGACGAGATCATCGAGCAGAT |
| | | CAGCGAGTTCTCCAAGAGAGTGATCCTGGCCGACGCTAATCTGGA |
| | | CAAGGTGCTGAGCGCCTACAACAAGCACAGAGACAAGCCTATCAG |
| | | AGAGCAGGCCGAGAATATCATCCACCTGTTTACCCTGACCAATCT |
| | | GGGAGCCCCTGCCGCCTTCAAGTACTTTGACACCACCATCGACCG |
| | | GAAGAGGTACACCAGCACCAAAGAGGTGCTGGACGCCACCCTGAT |
| | | CCACCAGAGCATCACCGGCCTGTACGAGACACGGATCGACCTGTC |
| | | TCAGCTGGGAGGCGACAGCGGAAGTGAGACCCCAGGTACATCCGA |
| | | ATCAGCAACGCCTGAAAGCACCGGTCGGACACTGGTGACCTTCAA |
| | | GGATGTATTTGTGGACTTCACCAGGGAGGAGTGGAAGCTGCTGGA |
| | | CACTGCTCAGCAGATCGTGTACAGAAATGTGATGCTGGAGAACTA |
| | | TAAGAACCTGGTTTCCTTGGGTTATCAGCTTACTAAGCCAGATGT |
| | | GATCCTCCGGTTGGAGAAGGGAGAAGAGCCCAGCGCTGATTACAA |
| | | AGATGATGACGATAAAGCCCCAAAAAAGAAGAGAAAGGTACCGAA |
| | | GAAAAAAAGAAAGGTCTGAGGATCCTGAGTCTAGAAATCAACCTC |
| | | TGGATTACAAAATTTGTGAAAGATTGACTGGTATTCTTAACTATG |
| | | TTGCTCCTTTTACGCTATGTGGATACGCTGCTTTAATGCCTTTGT |
| | | ATCATGCTATTGCTTCCCGTATGGCTTTCATTTTCTCCTCCTTGT |
| | | ATAAATCCTGGTTGCTGTCTCTTTATGAGGAGTTGTGGCCCGTTG |
| | | TCAGGCAACGTGGCGTGGTGTGCACTGTGTTTGCTGACGCAACCC |
| | | CCACTGGTTGGGGCATTGCCACCACCTGTCAGCTCCTTTCCGGGA |
| | | CTTTCGCTTTCCCCCTCCCTATTGCCACGGCGGAACTCATCGCCG |
| | | CCTGCCTTGCCCGCTGCTGGACAGGGGCTCGGCTGTTGGGCACTG |
| | | ACAATTCCGTGGTGTTGTCGGGGAAATCATCGTCCTTTCCTTGGC |
| | | TGCTCGCCTGTGTTGCCACCTGGATTCTGCGCGGGACGTCCTTCT |
| | | GCTACGTCCCTTCGGCCCTCAATCCAGCGGACCTTCCTTCCCGCG |
| | | GCCTGCTGCCGGCTCTGCGGCCTCTTCCGCGTCTTCGCCTTCGCC |
| | | CTCAGACGAGTCGGATCTCCCTTTGGGCCGCCTCCCCGCCTGTTA |
| | | ATT |
| 697 | Full Nucleic Acid | AGACCCAAGCTGGCTAGCCGCCACCATGGGTACC |
| | Sequence of 5'UTR | |
| 698 | Spacer Sequence for | GCAACACCGCCUAGACCGAC |
| | CLTA Guide RNA | |
| 699 | Tracr Sequence for | GUUUAAGAGCUAAGCUGGAAACAGCAUAGCAAGUUUAAAUAAGGC |
| | CLTA Guide RNA | UAGUCCGUUAUCAACUUGAAAAAGUGGCACCGAGUCGGUGCUUUU |
| | | UU |
| 700 | Target Site for | GCAACACCGCCTAGACCGAC |
| | CLTA Guide RNA | |
| 701 | Spacer Sequence for | ACUGCCUGGCUCACUCCUCC |
| | PCSK9 Guide RNA | |
| 702 | Target Site for | ACTGCCTGGCTCACTCCTCC |
| | PCSK9 Guide RNA | |

What is claimed is:

1. A fusion protein comprising one or more DNA methyltransferase (DNMT) domains and/or a recruiter domain that recruits a DNMT, two or more DNA-binding zinc finger domains, and one or more transcriptional repressor domains.

2. The fusion protein of claim 1, comprising from N-terminus to C-terminus, a DNMT domain and/or a recruiter domain that recruits a DNMT, a first DNA-binding zinc finger domain, a second DNA-binding zinc finger domain, and a transcriptional repressor domain.

3. The fusion protein of claim 1, comprising from N-terminus to C-terminus, a transcriptional repressor domain, a first DNA-binding zinc finger domain, a second zinc finger domain, and a DNMT domain and/or a recruiter domain that recruits a DNMT.

4. The fusion protein of claim 1, comprising from N-terminus to C-terminus, a DNMT domain and/or a recruiter domain that recruits a DNMT, a first DNA-binding zinc finger domain, a transcriptional repressor domain, and a second DNA-binding zinc finger domain.

5. The fusion protein of claim 1, comprising from N-terminus to C-terminus, a DNA-binding zinc finger domain, a transcriptional repressor domain, a second DNA-binding zinc-finger domain, and a DNMT domain and/or a recruiter domain that recruits a DNMT.

6. The fusion protein of claim 1, comprising from N-terminus to C-terminus, a first transcriptional repressor domain, a first DNA-binding zinc finger domain, a DNMT domain and/or a recruiter domain that recruits a DNMT, a second DNA-binding zinc finger domain, and a second transcriptional repressor domain.

7. The fusion protein of any one of claims 1-6, wherein at least one of the transcriptional repressor domains comprises a KRAB domain derived from KOX1, ZIM3, ZFP28, or ZN627.

8. The fusion protein of claim 7, wherein the KRAB domain comprises a sequence with at least 90% identity to a sequence selected from SEQ ID NOs: 89, 116, 245, and 255.

9. The fusion protein of any one of claims 1-6, wherein at least one of the transcriptional repressor domains comprises a fusion of the N- and C-terminal regions of ZIM3 and KOX1 KRAB, and optionally comprises the amino acid sequence of SEQ ID NO: 571 or 572.

10. The fusion protein of any one of claims 1-6, wherein at least one of the transcriptional repressor domains is derived from KAP1, MECP2, HP1a/CBX5, HP1b, CBX8, CDYL2, TOX, TOX3, TOX4, EED, EZH2, RBBP4, RCOR1, or SCML2.

11. The fusion protein of any one of claims 1-10, wherein the DNMT domain and/or the recruiter domain that recruits a DNMT comprises a DNMT3A domain and/or a DNMT3L domain, optionally wherein the recruited DNMT is a DNMT3A.

12. The fusion protein of claim 1 or claim 2, wherein the fusion comprises SEQ ID NO: 658 or a sequence at least 90% identical thereto.

13. The fusion protein of claim 12, wherein the fusion comprises SEQ ID NO: 658 or a sequence at least 95% identical thereto.

14. The fusion protein of claim 1 or claim 3, wherein the fusion comprises SEQ ID NO: 23 or a sequence at least 90% identical thereto.

15. The fusion protein of claim 14, wherein the fusion comprises SEQ ID NO: 23 or a sequence at least 95% identical thereto.

16. The fusion protein of claim 1 or claim 6, wherein the fusion comprises SEQ ID NO: 659 or a sequence at least 90% or 95% identical thereto.

17. The fusion protein of any one of claims 1-16, wherein the first DNA-binding zinc finger domain and the second DNA-binding zinc finger domain are the same.

18. The fusion protein of any one of claims 1-16, wherein the first DNA-binding zinc finger domain and the second DNA-binding zinc finger domain are different.

19. The fusion protein of any one of claims 1-11, wherein the fusion protein further comprises a third DNA-binding zinc finger domain.

20. The fusion protein of claim 19, wherein the first DNA-binding zinc finger domain, the second DNA-binding zinc finger domain, and the third DNA-binding zinc finger domain are the same.

21. The fusion protein of claim 19, wherein the first DNA-binding zinc finger domain and the second DNA-binding zinc finger domain are the same.

22. The fusion protein of claim 19, wherein the first DNA-binding zinc finger domain and the third DNA-binding zinc finger domain are the same.

23. The fusion protein of claim 19, wherein the second DNA-binding zinc finger domain and the third DNA-binding zinc finger domain are the same.

24. The fusion protein of claim 19, wherein each of the DNA-binding zinc finger domains is a different DNA-binding zinc finger domain.

25. The fusion protein of any one of claims 1-24, wherein the fusion protein targets multiple sites in the same genetic element.

26. The fusion protein of any one of claims 1-24, wherein the fusion protein targets two or more genetic elements.

27. The fusion protein of any one of claims 1-26, wherein the fusion protein increases DNA accessibility or availability for editing.

28. The fusion protein of any one of claims 1-27, wherein the fusion protein further comprises one or more linkers.

29. The fusion protein of claim 28, wherein the one or more linkers comprise XTEN linkers, optionally selected from XTEN80 and XTEN16.

30. The fusion protein of any one of claims 1-29, wherein the fusion protein further comprises one or more nuclear localization signals (NLS).

31. The fusion protein of claim 30, wherein at least one of the NLSs is an SV40 NLS.

32. The fusion protein of claim 1, comprising from N-terminus to C-terminus, two nuclear localization signals (NLSs), a DNMT domain and/or a recruiter domain that recruits a DNMT, a first linker, a first DNA-binding zinc finger domain, a second linker, a second DNA-binding zinc finger domain, a third NLS, a third linker, a transcriptional repressor domain, and two NLSs.

33. The fusion protein of claim 1, comprising from N-terminus to C-terminus, two nuclear localization signals (NLSs), a transcriptional repressor domain, a first linker, a third NLS, a first DNA-binding zinc finger domain, a second linker, a second DNA-binding zinc finger domain, a third linker, a DNMT domain and/or a recruiter domain that recruits a DNMT, and two NLSs.

34. The fusion protein of claim 32 or claim 33, wherein at least one of the linkers comprises an XTEN linker, optionally an XTEN80 linker or an XTEN16 linker.

35. The fusion protein of claim 1, comprising from N-terminus to C-terminus, two nuclear localization signals, a transcriptional repressor domain, a first linker, a first DNA-binding zinc finger domain, a second linker, a DNMT domain and/or a recruiter domain that recruits a DNMT, a third linker, a third NLS, a second DNA-binding zinc finger domain, a fourth linker, a second transcriptional repressor domain, and two additional NLSs.

36. The fusion protein of claim 1, comprising from N-terminus to C-terminus, two nuclear localization signals (NLSs), a transcriptional repressor domain, a first linker, a first DNA-binding zinc finger domain, a third NLS, a second linker, a DNMT domain and/or a recruiter domain that recruits a DNMT, a third linker, a second DNA-binding zinc finger domain, a fourth linker, a second transcriptional repressor domain, and two NLSs.

37. The fusion protein of claim 35 or claim 36, wherein at least one of the linkers comprises an XTEN linker, optionally an XTEN80 linker or an XTEN16 linker.

38. The fusion protein of any one of claims 1-37, further comprising a post-transcriptional regulatory element, optionally a woodchuck hepatitis virus post-transcriptional regulatory element (WPRE).

39. A nucleic acid encoding the fusion protein of any one of claims 1-38.

40. A cell comprising the fusion protein of any one of claims 1-38 or the nucleic acid of claim 39, or progeny of the cell.

41. The cell of claim 40, wherein the cell is a human cell.

42. A composition comprising the fusion protein of any one of claims 1-38, the nucleic acid of claim 39, or the cell of claim 40 or claim 41.

43. The composition of claim 42, further comprising a pharmaceutically acceptable excipient.

44. A method comprising administering to a subject the fusion protein of any one of claims 1-38, the nucleic acid of claim 39, the cell of claim 40 or 41, or the pharmaceutical composition of claim 42 or 43.

45. The method of claim 44, wherein the subject is human.

46. A system for modulating transcription of one or more human genes in a human cell, comprising
i) the fusion protein of any one of claims 1-38, or
ii) the nucleic acid of claim 39.

47. The system of claim 46, wherein the system modulates transcription of two human genes.

48. The system of claim 46 or claim 47, wherein the fusion protein increases DNA accessibility or availability for editing.

49. The system of any one of claims 46-48, wherein the system causes durable silencing of one or more human genes.

50. The system of any one of claims 46-49, wherein the system increases transcription of one or more human genes.
